(19) United States
(12) Patent Application Publication
(10) Pub. No.: US 2026/0055097 A9
(43) Pub. Date: Feb. 26, 2026

(54) NRF2 PROTEIN DEGRADERS

(71) Applicant: Ganymede Oncology, Inc., San Diego, CA (US)

(72) Inventors: John Edwin MUNROE, San Diego, CA (US); Robert Christian Wild, Murrieta, CA (US)

(21) Appl. No.: 18/999,301

(22) Filed: Dec. 23, 2024

Related U.S. Application Data
(63) Continuation-in-part of application No. PCT/US2023/069147, filed on Jun. 27, 2023.
(60) Provisional application No. 63/355,834, filed on Jun. 27, 2022.

(51) Int. Cl. C07D 417/14; A61K 31/454; A61K 31/4545; A61K 31/496; A61P 35/00

(52) U.S. Cl. CPC C07D 417/14; A61K 31/454; A61K 31/4545; A61K 31/496; A61P 35/00

(57) ABSTRACT

The present disclosure provides compounds represented by Formula A-I:

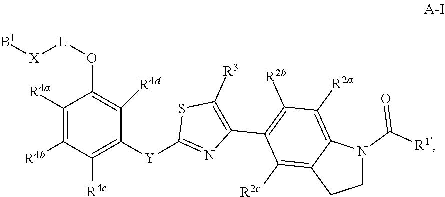

and the salts or solvates thereof, wherein R¹, R^{2a}, R^{2b}, R^{2c}, R³, R^{4a}, R^{4b}, R^{4c}, R^{4d}, L, X, Y, and B¹ are as defined in the specification. Compounds having Formula A-I are nuclear factor erythroid 2-related factor 2 (Nrf2) degraders useful for the treatment of cancer and other diseases.

### BACKGROUND

### Field

The present disclosure provides heterobifunctional small molecules as nuclear factor erythroid 2 related factor 2 (Nrf2) protein degraders. Nrf2 degraders are useful for the treatment of cancer and other diseases.

### Background

The Proteolysis Targeting Chimera (PROTAC) strategy utilizes the proteasome-mediated proteolysis to induce targeted protein degradation. Raina et al., Proc Natl Acad Sci USA. 2016, 113, 7124-7129; Zhou et al., J. Med. Chem. 2018, 61, 462-481. A PROTAC molecule is a heterobifunctional small molecule containing one ligand, which binds to the target protein of interest, and a second ligand for an E3 ligase system, tethered together by a chemical linker. Bondeson, D. P.; Crews, C. M. Targeted Protein Degradation by Small Molecules. Annu Rev Pharmacol Toxicol. 2017, 37, 107-123.

Nuclear factor erythroid 2 related factor 2 (Nrf2) is a transcription factor that regulates the expression of antioxidant genes. Both Kelch-like ECH-associated protein 1 (Keap1) mutations and Nrf2 mutations contribute to the activation of Nrf2 in cancer cells. Nrf2 activity, for example, is associated with poor prognosis in NSCLC. Zhao et al., Front. Oncol. 10:578315. doi: 10.3389/fonc.2020.578315.

The Nrf2 pathway also plays a role in neurodegeneration, diabetes, cardiovascular disease, kidney disease, and liver disease. Nrf2 levels vary significantly depending on physiological, temporal and pathological context. Dodson et al., Annu Rev Pharmacol Toxicol 59:555-575 (2019). Nrf2 activation promotes metabolic reprogramming. Inhibition or degradation of Nrf2 can alter metabolic processes and thus suppress tumor growth, prevent metastasis, and/or increase sensitivity to chemotherapy. There is a need in the art for Nrf2 degraders to treat cancer and other diseases.

### BRIEF SUMMARY

In one aspect, the present disclosure provides heterobifunctional small molecules represented by Formula A-I, I, and II below, and the pharmaceutically acceptable salts and solvates, e.g., hydrates, thereof. These compounds, and the salts and solvates thereof, are collectively referred to herein as “Compounds of the Disclosure” or individually as a “Compound of the Disclosure.” Compounds of the Disclosure are Nrf2 degraders and are thus useful in treating diseases or conditions wherein degradation of the Nrf2 protein provides a therapeutic benefit to a subject.

In another aspect, the present disclosure provides methods of treating a condition or disease by administering a therapeutically effective amount of a Compound of the Disclosure to a subject, e.g., a human cancer patient, in need thereof. The disease or condition treatable by degradation of Nrf2 is, for example, a cancer, a neurodegenerative disease, diabetes, a cardiovascular disease, a kidney disease, or a liver disease.

In another aspect, the present disclosure provides a method of degrading, e.g., reducing the level of, Nrf2 protein in a subject in need thereof, comprising administering to the subject an effective amount of a Compound of the Disclosure.

In another aspect, the present disclosure provides a pharmaceutical composition comprising a Compound of the Disclosure and an excipient and/or pharmaceutically acceptable carrier.

In another aspect, the present disclosure provides a composition comprising a Compound of the Disclosure and an excipient and/or pharmaceutically acceptable carrier for use treating diseases or conditions wherein degradation of the Nrf2 provides a benefit, e.g., cancer.

In another aspect, the present disclosure provides a composition comprising: (a) a Compound of the Disclosure; (b) a second therapeutically active agent; and (c) optionally an excipient and/or pharmaceutically acceptable carrier.

In another aspect, the present disclosure provides a Compound of the Disclosure for use in treatment of a disease or condition of interest, e.g., cancer.

In another aspect, the present disclosure provides a use of a Compound of the Disclosure for the manufacture of a medicament for treating a disease or condition of interest, e.g., cancer.

In another aspect, the present disclosure provides a kit comprising a Compound of the Disclosure, and, optionally, a packaged composition comprising a second therapeutic agent useful in the treatment of a disease or condition of interest, and a package insert containing directions for use in the treatment of a disease or condition, e.g., cancer.

In another aspect, the present disclosure provides methods of preparing Compounds of the Disclosure.

In another aspect, the present disclosure provides compounds represented by Formula III below, and the pharmaceutically acceptable salts and solvates, e.g., hydrates, thereof. Compounds having Formula III can be used, for example, as synthetic intermediates to prepare Compounds of the Disclosure.

Additional embodiments and advantages of the disclosure will be set forth, in part, in the description that follows, and will flow from the description, or can be learned by practice of the disclosure. The embodiments and advantages of the disclosure will be realized and attained by means of the elements and combinations particularly pointed out in the appended claims. It is to be understood that both the foregoing summary and the following detailed description are exemplary and explanatory only, and are not restrictive of the invention as claimed.

### DETAILED DESCRIPTION

### I. Compounds of the Disclosure

In one embodiment, Compounds of the Disclosure are compounds of Formula A-1:

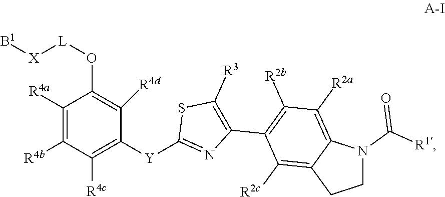

or a pharmaceutically acceptable salt or solvate thereof, wherein:
   R¹ is selected from the group consisting of optionally substituted C₁-C₆ alkyl, optionally substituted C₃-C₈ cycloalkyl, optionally substituted phenyl, and optionally substituted 5- to 9-membered heteroaryl;
   R^{2a}, R^{2b}, and R^{2c} are independently selected from the group consisting of hydrogen, C₁-C₄ alkyl, C₁-C₄ haloalkyl, C₁-C₄ alkoxy, C₁-C₄ haloalkoxy, cyano, hydroxy, —S(═O)₂CH₃, and halo;
   R³ is selected from the group consisting of hydrogen, C₁-C₄ alkyl, C₁-C₄ haloalkyl, C₁-C₄ alkoxy, C₁-C₄ haloalkoxy, cyano, hydroxy, —S(═O)₂CH₃, and halo;
   Y is selected from the group consisting of —\hat{ }N(H)C(═O)—, —\hat{ }N(H)C(═O)CH₂—, —\hat{ }C(═O)N(H)— and —\hat{ }C(═O)N(H)CH₂—;
   wherein the bond marked with a is attached to the thiazole;
   R^{4a}, R^{4b}, R^{4c}, and R^{4d} are independently selected from the group consisting of hydrogen. C₁-C₄ alkyl, C₁-C₄ haloalkyl, C₁-C₄ alkoxy, C₁-C₄ haloalkoxy, cyano, hydroxy, —S(═O)₂CH₃, and halo;
   L is selected from the group consisting of —(CH₂)ₘ—, —*(CH₂)ₙ(OCH₂CH₂)ₒ—, and —(CH₂)ₚ—Z—(CH₂)_{q}—;
   wherein the carbon marked with an “*” is attached to X;
   m is 2, 3, 4, 5, 6, 7, 8, 9, 10, 11, or 12;
   n is 2, 3, or 4;
   is 1, 2, 3, 4, 5, 6, 7, 8, 9, 10, 11, or 12;
   p is 0, 1, 2, 3, 4, 5, 6, 7, 8, 9, 10, 11, or 12;
   q is 1, 2, 3, 4, 5, 6, 7, 8, 9, 10, 11, or 12;
   Z is selected from the group consisting of —(CR^{6a}R^{6b})— and —N(R⁷)—;
   R^{6a} is selected from the group consisting of halo, hydroxyl, C₁-C₆ alkyl, C₁-C₄ haloalkyl, optionally substituted C₃-C₈ cycloalkyl, substituted optionally C₄-C₈ heterocyclo, optionally substituted phenyl, and optionally substituted 5- to 9-membered heteroaryl;
   R^{6b} is selected from the group consisting of hydrogen and C₁-C₆ alkyl;
   R⁷ is selected from the group consisting of hydrogen, C₁-C₆ alkyl, and C₁-C₄ haloalkyl;
   X is selected from the group consisting of —O—, —NH—,

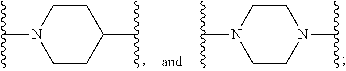 [SMILES: *C1CCN(*)CC1 ; *N1CCN(*)CC1]

B¹ is selected from the group consisting of:

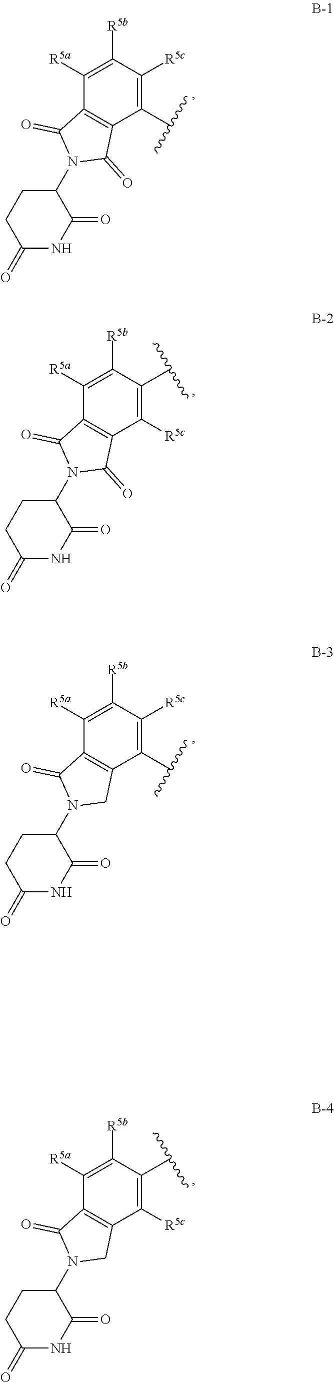

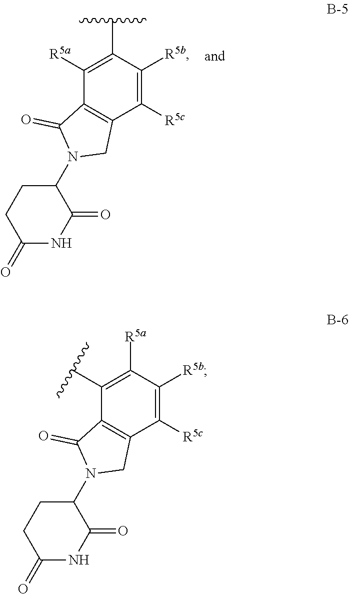

and
R^{5a}, R^{5b}, and R^{5c} are independently selected from the group consisting of hydrogen, C₁-C₄ alkyl, C₁-C₄ haloalkyl, C₁-C₄ alkoxy, C₁-C₄ haloalkoxy, cyano, hydroxy, —S(═O)₂CH₃, and halo.

In another embodiment, Compounds of the Disclosure are compounds of Formula I:

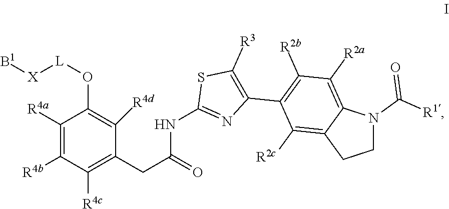

or a pharmaceutically acceptable salt or solvate thereof.

In another embodiment, Compounds of the Disclosure are compounds of Formula II:

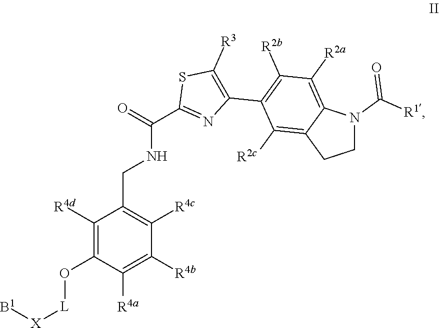

or a pharmaceutically acceptable salt or solvate thereof.

In another embodiment, Compounds of the Disclosure are compounds of any one of Formula A-I, I, or II, or a pharmaceutically acceptable salt or solvate thereof, wherein R¹ is optionally substituted C₃-C₈ cycloalkyl. In another embodiment, R¹ is cyclopropyl, cyclobutyl, cyclopentyl, or cyclohexyl. In another embodiment, R¹ is cyclopropyl.

In another embodiment, Compounds of the Disclosure are compounds of any one of Formula A-I, I, or II, or a pharmaceutically acceptable salt or solvate thereof, wherein R¹ is optionally substituted phenyl. In another embodiment, R¹ is:

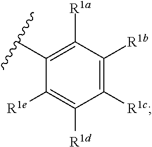

and
R^{1a}, R^{1b}, R^{1c}, R^{1d}, and R^{1e} are independently selected from the group consisting of hydrogen, C₁-C₄ alkyl, C₁-C₄ haloalkyl, C₁-C₄ alkoxy, C₁-C₄ haloalkoxy, cyano, hydroxy, —S(═O)₂CH₃, and halo. In another embodiment, R^{1b}, R^{1c}, R^{1d}, and R^{1e} are hydrogen. In another embodiment, R^{1a} is C₁-C₄ alkyl. In another embodiment, R^{1a} is methyl.

In another embodiment, Compounds of the Disclosure are compounds of any one of Formula A-I, I, or II, or a pharmaceutically acceptable salt or solvate thereof, wherein R¹ is optionally substituted 5- to 9-membered heteroaryl. In another embodiment, R¹ is optionally substituted imidazole. In another embodiment, R¹ is:

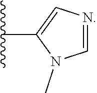 [SMILES: *c1cncn1C]

In another embodiment, Compounds of the Disclosure are compounds of any one of Formula A-I, I, or II, or a pharmaceutically acceptable salt or solvate thereof, wherein R^{2a}, R^{2b}, and R^{2c} are hydrogen.

In another embodiment, Compounds of the Disclosure are compounds of any one of Formula A-I, I, or II, or a pharmaceutically acceptable salt or solvate thereof, wherein R³ is C₁-C₄ alkyl. In another embodiment, R³ is methyl.

In another embodiment, Compounds of the Disclosure are compounds of any one of Formula A-I, I, or II, or a pharmaceutically acceptable salt or solvate thereof wherein R^{4a} is selected from the group consisting of hydrogen and fluoro.

In another embodiment, Compounds of the Disclosure are compounds of any one of Formula A-I, I, or II, or a pharmaceutically acceptable salt or solvate thereof, wherein R^{4b} is selected from the group consisting of hydrogen and fluoro.

In another embodiment, Compounds of the Disclosure are compounds of any one of Formula A-I, I, or II, or a pharmaceutically acceptable salt or solvate thereof, wherein R^{4c} is selected from the group consisting of hydrogen and fluoro.

In another embodiment, Compounds of the Disclosure are compounds of any one of Formula A-I, I, or II, or a pharmaceutically acceptable salt or solvate thereof, wherein R^{4b}, R^{4c}, and R^{4d} are hydrogen.

In another embodiment, Compounds of the Disclosure are compounds of any one of Formula A-I, I, or H, or a pharmaceutically acceptable salt or solvate thereof, wherein L is selected from the group consisting of —(CH₂)ₘ— and —*(CH₂)ₙ(OCH₂CH₂)ₒ—.

In another embodiment, Compounds of the Disclosure are compounds of any one of Formula A-I, I, or II, or a pharmaceutically acceptable salt or solvate thereof, wherein L is —(CH₂)ₘ—. In another embodiment, m is 1. In another embodiment, m is 2. In another embodiment, m is 3. In another embodiment, m is 4. In another embodiment, m is 5. In another embodiment, m is 6.

In another embodiment, Compounds of the Disclosure are compounds of any one of Formula A-I, I, or II, or a pharmaceutically acceptable salt or solvate thereof, wherein L is —*(CH₂)ₙ(OCH₂CH₂)ₒ—. In another embodiment, n is 2. In another embodiment, n is 3. In another embodiment, o is 1. In another embodiment, o is 2. In another embodiment, o is 3. In another embodiment, o is 4. In another embodiment, o is 5. In another embodiment, o is 6.

In another embodiment, Compounds of the Disclosure are compounds of any one of Formula A-I, I, or H, or a pharmaceutically acceptable salt or solvate thereof, wherein L is —(CH₂)ₚ—Z—CH₂)_{q}—. In another embodiment, p is 0. In another embodiment, p is 1. In another embodiment, p is 2. In another embodiment, p is 3. In another embodiment, p is 4. In another embodiment, q is 1. In another embodiment, q is 2. In another embodiment, q is 3. In another embodiment, q is 4.

In another embodiment, Compounds of the Disclosure are compounds of any one of Formula A-I, I, or II, or a pharmaceutically acceptable salt or solvate thereof, wherein Z is —(CR^{6a}R^{6b}).

In another embodiment, Compounds of the Disclosure are compounds of any one of Formula A-I, I, or II, or a pharmaceutically acceptable salt or solvate thereof, wherein Z is:

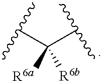

In another embodiment, Compounds of the Disclosure are compounds of any one of Formula A-I, I, or II, or a pharmaceutically acceptable salt or solvate thereof, wherein Z is:

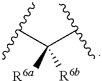

In another embodiment, Compounds of the Disclosure are compounds of Formula A-I, I, or II, or a pharmaceutically acceptable salt or solvate thereof, wherein Z is —N(R⁷)—.

In another embodiment, Compounds of the Disclosure are compounds of any one of Formula A-I, I, or II, or a pharmaceutically acceptable salt or solvate thereof, wherein R^{6a} is halo. In another embodiment, R^{6a} is hydroxyl. In another embodiment, R^{6a} is C₁-C₆ alkyl. In another embodiment, R^{6a} is C₁-C₄ haloalkyl. In another embodiment, R^{6a} is an optionally substituted C₃-C₈ cycloalkyl. In another embodiment, R^{6a} is an optionally substituted C₄-C₈ heterocyclo. In another embodiment, R^{6a} is an optionally substituted phenyl. In another embodiment, R^{6a} is an optionally substituted 5- to 9-membered heteroaryl.

In another embodiment, Compounds of the Disclosure are compounds of any one of Formula A-I, I, or II, or a pharmaceutically acceptable salt or solvate thereof, wherein R^{6b} is hydrogen. In another embodiment, R^{6b} is C₁-C₆ alkyl.

In another embodiment, Compounds of the Disclosure are compounds of any one of Formula A-I, I, or II, or a pharmaceutically acceptable salt or solvate thereof, wherein R⁷ is hydrogen. In another embodiment, R⁷ is C₁-C₆ alkyl. In another embodiment, R⁷ is C₁-C₄ haloalkyl.

In another embodiment, Compounds of the Disclosure are compounds of any one of Formula A-I, I, or II, or a pharmaceutically acceptable salt or solvate thereof, wherein X is —O—.

In another embodiment, Compounds of the Disclosure are compounds of any one of Formula A-I, I, or II, or a pharmaceutically acceptable salt or solvate thereof, wherein X is —NH—.

In another embodiment, Compounds of the Disclosure are compounds of any one of Formula A-I, I, or II, or a pharmaceutically acceptable salt or solvate thereof, wherein X is:

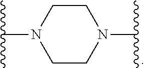 [SMILES: *N1CCN(*)CC1]

In another embodiment, Compounds of the Disclosure are compounds of any one of Formula A-I, I, or II, or a pharmaceutically acceptable salt or solvate thereof, wherein X is:

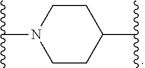 [SMILES: *C1CCN(*)CC1]

In another embodiment, Compounds of the Disclosure are compounds of any one of Formula A-I, I, or II, or a pharmaceutically acceptable salt or solvate thereof, wherein B¹ is B-1.

In another embodiment, Compounds of the Disclosure are compounds of any one of Formula A-I, I, or II, or a pharmaceutically acceptable salt or solvate thereof, wherein B¹ is B-2.

In another embodiment, Compounds of the Disclosure are compounds of any one of Formula A-I, I, or II, or a pharmaceutically acceptable salt or solvate thereof, wherein B¹ is B-3.

In another embodiment, Compounds of the Disclosure are compounds of any one of Formula A-I, I, or II, or a pharmaceutically acceptable salt or solvate thereof, wherein B¹ is B-4.

In another embodiment, Compounds of the Disclosure are compounds of any one of Formula A-I, 1, or II, or a pharmaceutically acceptable salt or solvate thereof wherein B¹ is B-5.

In another embodiment, Compounds of the Disclosure are compounds of any one of Formula A-I, I, or II, or a pharmaceutically acceptable salt or solvate thereof, wherein B¹ is B-6.

In another embodiment, Compounds of the Disclosure are compounds of any one of Formula A-I, I, or II, or a pharmaceutically acceptable salt or solvate thereof, wherein R^{5a}, R^{5b}, and R^{5c} are independently selected from the group consisting of hydrogen and fluoro. In another embodiment, R^{5a}, R^{5b}, and R^{5c} are hydrogen.

In another embodiment, Compounds of the Disclosure are compounds of any one of Formula A-I, I, or II, or or a pharmaceutically acceptable salt or solvate thereof, wherein B is selected from the group consisting of:

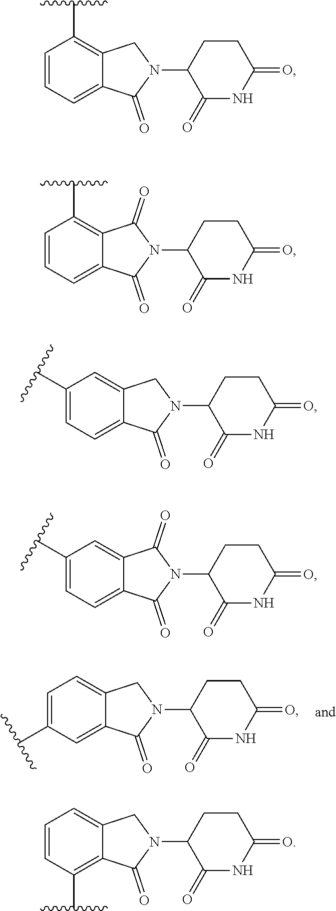 [SMILES: *c1cccc2c1CN(C1CCC(=O)NC1=O)C2=O ; *c1cccc2c1C(=O)N(C1CCC(=O)NC1=O)C2=O ; *c1ccc2c(c1)CN(C1CCC(=O)NC1=O)C2=O ; *c1ccc2c(c1)C(=O)N(C1CCC(=O)NC1=O)C2=O ; *c1ccc2c(c1)C(=O)N(C1CCC(=O)NC1=O)C2 ; *c1cccc2c1C(=O)N(C1CCC(=O)NC1=O)C2]

In another embodiment, Compounds of the Disclosure are compounds of any one of Formula A-I, I, or II, or a pharmaceutically acceptable salt or solvate thereof, wherein B is selected from the group consisting of:

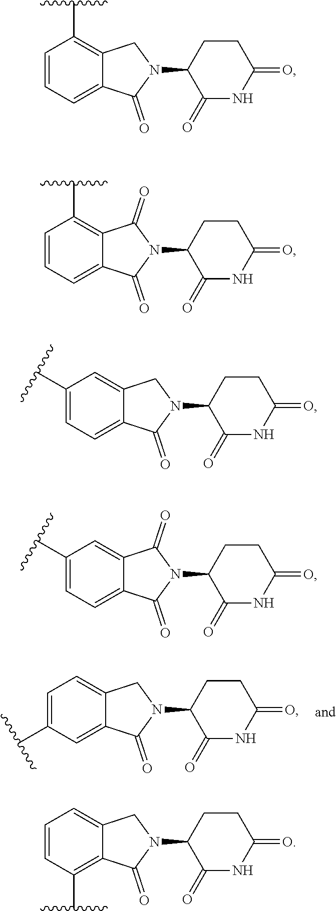 [SMILES: *c1cccc2c1CN([C@H]1CCC(=O)NC1=O)C2=O ; *c1cccc2c1C(=O)N([C@H]1CCC(=O)NC1=O)C2=O ; *c1ccc2c(c1)CN([C@H]1CCC(=O)NC1=O)C2=O ; *c1ccc2c(c1)C(=O)N([C@H]1CCC(=O)NC1=O)C2=O ; *c1ccc2c(c1)C(=O)N([C@H]1CCC(=O)NC1=O)C2 ; *c1cccc2c1C(=O)N([C@H]1CCC(=O)NC1=O)C2]

In another embodiment, Compounds of the Disclosure are compounds of any one of Formula A-I, I, or II, or a pharmaceutically acceptable salt or solvate thereof, wherein B¹ is selected from the group consisting of:

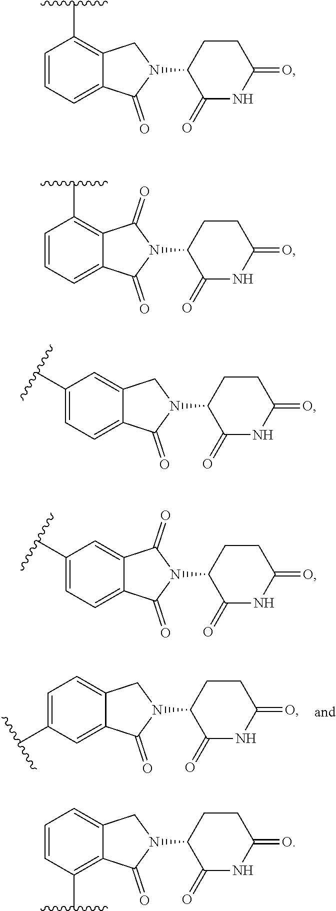 [SMILES: *c1cccc2c1CN([C@@H]1CCC(=O)NC1=O)C2=O ; *c1cccc2c1C(=O)N([C@@H]1CCC(=O)NC1=O)C2=O ; *c1ccc2c(c1)CN([C@@H]1CCC(=O)NC1=O)C2=O ; *c1ccc2c(c1)C(=O)N([C@@H]1CCC(=O)NC1=O)C2=O ; *c1ccc2c(c1)C(=O)N([C@@H]1CCC(=O)NC1=O)C2 ; *c1cccc2c1C(=O)N([C@@H]1CCC(=O)NC1=O)C2]

In another embodiment, Compounds of the Disclosure are compounds of any one of Formula A-I, I, or II, or a pharmaceutically acceptable salt or solvate thereof, wherein B¹ is:

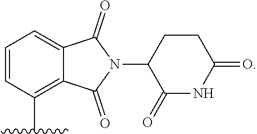 [SMILES: *c1cccc2c1C(=O)N(C1CCC(=O)NC1=O)C2=O]

In another embodiment, Compounds of the Disclosure are compounds of any one of Formula A-I, I, or II, or a pharmaceutically acceptable salt or solvate thereof, wherein B¹ is:

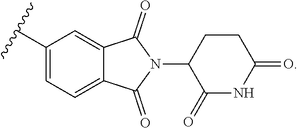 [SMILES: *c1ccc2c(c1)C(=O)N(C1CCC(=O)NC1=O)C2=O]

In one embodiment, the disclosure provides compounds of Formula III:

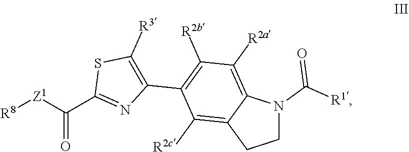

or a pharmaceutically acceptable salt thereof, wherein:
   R^{1′} is selected from the group consisting of optionally substituted C₁-C₆ alkyl, optionally substituted C₃-C₈ cycloalkyl, optionally substituted phenyl, and optionally substituted 5- to 9-membered heteroaryl;
   R^{2a′}, R^{2′}, and R^{2c′} are independently selected from the group consisting of hydrogen, C₁-C₄ alkyl, C₁-C₄ haloalkyl, C₁-C₄ alkoxy, C₁-C₄ haloalkoxy, cyano, hydroxy, —S(═O)₂CH₃, and halo;
   R^{3′} is selected from the group consisting of hydrogen, C₁-C₄ alkyl, C₁-C₄ haloalkyl, C₁-C₄ alkoxy, C₁-C₄ haloalkoxy, cyano, hydroxy, —S(═O)₂CH₃, and halo;
   Z¹ is —O—; and
   R⁸ is hydrogen, C₁-C₆ alkyl, or aralkyl; or
   Z¹ is —N(H)—; and
   R⁸ is:

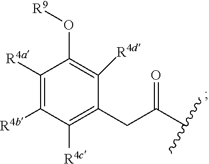

R^{4a′}, R^{4b′}, R^{4c′}, and R^{4d′} are independently selected from the group consisting of hydrogen, C₁-C₄ alkyl, C₁-C₄ haloalkyl, C₁-C₄ alkoxy, C₁-C₄ haloalkoxy, cyano, hydroxy, —S(═O)₂CH₃, and halo;
R⁹ is selected from the group consisting of hydrogen and -L¹-X¹;
X¹ is selected from the group consisting of —OR¹⁰ and —NR^{11a}R^{11b};
R¹⁰ is hydrogen;
R^{11a} is selected from the group consisting of hydrogen and —C(═O)OtBu;
R^{11b} is selected from the group consisting of hydrogen and C₁-C₄ alkyl;
L¹ is selected from the group consisting of —(CH₂)_{m′}—, —*(CH₂)_{n′}(OCH₂CH₂)_{o′}—, and —(CH₂)_{p′}—Z²—(CH₂)_{q′};
wherein the carbon marked with an “*” is attached to X¹;
m′ is 2, 3, 4, 5, 6, 7, 8, 9, 10, 11, or 12;
n′ is 2, 3, or 4;
o′ is 1, 2, 3, 4, 5, 6, 7, 8, 9, 10, 11, or 12;
p′ is 0, 1, 2, 3, 4, 5, 6, 7, 8, 9, 10, 11, or 12;
q′ is 1, 2, 3, 4, 5, 6, 7, 8, 9, 10, 11, or 12;
Z² is selected from the group consisting of —(CR^{6a′}R^{6b′})— and —N(R^{7′})—;
R^{6a′} is selected from the group consisting of halo, hydroxyl, C₁-C₆ alkyl, C₁-C₄ haloalkyl, optionally substituted C₃-C₈ cycloalkyl, substituted optionally C₄-C₈ heterocyclo, optionally substituted phenyl, and optionally substituted 5- to 9-membered heteroaryl;
R^{6b′} is selected from the group consisting of hydrogen and C₁-C₈ alkyl; and
R^{7′} is selected from the group consisting of hydrogen, C₁-C₆ alkyl, and C₁-C₄ haloalkyl.

In another embodiment, the disclosure provides compounds of Formula III, or a pharmaceutically acceptable salt or solvate thereof, wherein R^{1′} is optionally substituted C₃-C₈ cycloalkyl. In another embodiment, R^{1′} is cyclopropyl, cyclobutyl, cyclopentyl, or cyclohexyl. In another embodiment, R^{1′}, is cyclopropyl.

In another embodiment, the disclosure provides compounds of Formula III, or a pharmaceutically acceptable salt or solvate thereof, wherein R^{1′} is optionally substituted phenyl. In another embodiment, R^{1′} is:

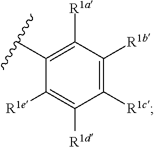

R^{1a′}, R^{1b′}, R^{1c′}, R^{1d′}, and R^{1e′} are independently selected from the group consisting of hydrogen, C₁-C₄ alkyl, C₁-C₄ haloalkyl, C₁-C₄ alkoxy, C₁-C₄ haloalkoxy, cyano, hydroxy, —S(═O)₂CH₃, and halo. In another embodiment, R^{1b′}, R^{1c′}, R^{1d′}, and R^{1e′} are hydrogen. In another embodiment, R^{1a′} is C₁-C₄ alkyl. In another embodiment, R^{1a′} is methyl.

In another embodiment, the disclosure provides compounds of Formula III, or a pharmaceutically acceptable salt or solvate thereof, wherein R^{1′} is optionally substituted 5- to 9-membered heteroaryl. In another embodiment, R^{1′} is optionally substituted imidazole. In another embodiment, R^{1′} is:

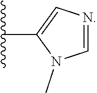 [SMILES: *c1cncn1C]

In another embodiment, the disclosure provides compounds of Formula III, or a pharmaceutically acceptable salt or solvate thereof, wherein R^{2a′}, R^{2b′}, and R^{2c′} are hydrogen.

In another embodiment, the disclosure provides compounds of Formula III, or a pharmaceutically acceptable salt or solvate thereof, wherein R^{3′} is C₁-C₄ alkyl. In another embodiment, R^{3′} is methyl.

In another embodiment, the disclosure provides compounds of Formula III, or a pharmaceutically acceptable salt or solvate thereof, wherein Z¹ is —O—.

In another embodiment, the disclosure provides compounds of Formula III, or a pharmaceutically acceptable salt or solvate thereof, wherein Z¹ is —N(H)—.

In another embodiment, the disclosure provides compounds of Formula III, or a pharmaceutically acceptable salt or solvate thereof, wherein R⁸ is hydrogen. In another embodiment, R⁸ is C₁-C₆ alkyl. In another embodiment, R⁸ is aralkyl. In another embodiment, R¹ is:

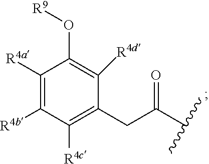

R^{4a′}, R^{4b′}, R^{4c′}, and R^{4d′} are independently selected from the group consisting of hydrogen, C₁-C₄ alkyl, C₁-C₄ haloalkyl, C₁-C₄ alkoxy, C₁-C₄ haloalkoxy, cyano, hydroxy, —S(═O)₂CH₃, and halo.

In another embodiment, the disclosure provides compounds of Formula III, or a pharmaceutically acceptable salt or solvate thereof, wherein R⁹ is hydrogen.

In another embodiment, the disclosure provides compounds of Formula III, or a pharmaceutically acceptable salt or solvate thereof, wherein R⁹ is -L¹-X¹.

In another embodiment, the disclosure provides compounds of Formula III, or a pharmaceutically acceptable salt or solvate thereof, wherein X¹ is —OR¹⁰. In another embodiment, X¹ is —NR^{11a}R^{11b}.

In another embodiment, the disclosure provides compounds of Formula III, or a pharmaceutically acceptable salt or solvate thereof, wherein R¹⁰ is hydrogen.

In another embodiment, the disclosure provides compounds of Formula III, or a pharmaceutically acceptable salt or solvate thereof, wherein R^{11a} is hydrogen. In another embodiment, R^{11a} is —C(═O)OtBu.

In another embodiment, the disclosure provides compounds of Formula III, or a pharmaceutically acceptable salt or solvate thereof, wherein R^{11b} is hydrogen. In another embodiment, R^{11b} is C₁-C₄ alkyl.

In another embodiment, the disclosure provides compounds of Formula II, or a pharmaceutically acceptable salt or solvate thereof, wherein L¹ is —(CH₂)_{m′}—. In another embodiment, L¹ is —*(CH₂)ₙ(OCH₂CH₂)_{o′}—. In another embodiment, L¹ is —(CH₂)_{p′}—Z²—(CH₂)_{q′}—.

In another embodiment, the disclosure provides compounds of Formula I, or a pharmaceutically acceptable salt or solvate thereof, wherein L¹ is —(CH₂)_{m′}—. In another embodiment, m′ is 2. In another embodiment, m′ is 3. In another embodiment, m′ is 4. In another embodiment, m′ is 5. In another embodiment, m′ is 6.

In another embodiment, the disclosure provides compounds of Formula III, or a pharmaceutically acceptable salt or solvate thereof, wherein L¹ is —*(CH₂)_{n′}(OCH₂CH₂)_{o′}—. In another embodiment, n′ is 2. In another embodiment, n′ is 3. In another embodiment, n is 4. In another embodiment, o′ is 1. In another embodiment, o′ is 2. In another embodiment, o′ is 3. In another embodiment, o′ is 4. In another embodiment, 6′ is 5. In another embodiment, o′ is 6.

In another embodiment, the disclosure provides compounds of Formula III, or a pharmaceutically acceptable salt or solvate thereof, wherein L¹ is —(CH₁₂)_{p′}—Z²—(CH₂)_{q′}—. In another embodiment, p′ is 0. In another embodiment, p′ is 1. In another embodiment, p′ is 2. In another embodiment, p′ is 3. In another embodiment, p′ is 4. In another embodiment, q′ is 1. In another embodiment, q′ is 2. In another embodiment, q′ is 3. In another embodiment, q′ is 4.

In another embodiment, the disclosure provides compounds of Formula III, or a pharmaceutically acceptable salt or solvate thereof, wherein Z² is —(CR^{6a′}R^{6b′})—.

In another embodiment, the disclosure provides compounds of Formula III, or a pharmaceutically acceptable salt or solvate thereof, wherein Z² is:

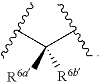

In another embodiment, the disclosure provides compounds of Formula III, or a pharmaceutically acceptable salt or solvate thereof, wherein Z² is:

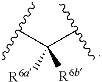

In another embodiment, the disclosure provides compounds of Formula III, or a pharmaceutically acceptable salt or solvate thereof, wherein Z² is —N(R^{7′})—.

In another embodiment, the disclosure provides compounds of Formula III, or a pharmaceutically acceptable salt or solvate thereof, wherein R^{6a′} is halo. In another embodiment, R^{6a′} is hydroxyl. In another embodiment, R^{6a′} is C₁-C₆ alkyl. In another embodiment, R^{6a′} is C₁-C₄ haloalkyl. In another embodiment, R^{6a′} is an optionally substituted C₃-C₈ cycloalkyl. In another embodiment, R^{6a′} is an optionally substituted C₄-C₈ heterocyclo. In another embodiment, R^{6a′} is an optionally substituted phenyl. In another embodiment, R^{6a′} is an optionally substituted 5- to 9-membered heteroaryl.

In another embodiment, the disclosure provides compounds of Formula III, or a pharmaceutically acceptable salt or solvate thereof, wherein R^{6′} is hydrogen. In another embodiment, R^{6b′} is C₁-C₆ alkyl.

In another embodiment, the disclosure provides compounds of Formula III, or a pharmaceutically acceptable salt or solvate thereof, wherein R^{7′} is hydrogen. In another embodiment, R^{7′} is C₁-C₆ alkyl. In another embodiment, R^{7′} is C₁-C₄ haloalkyl.

In another embodiment, Compounds of the Disclosure are any one or more of the compounds of Table 1, or a pharmaceutically acceptable salt or solvate thereof.

In another embodiment, the disclosure provides any one or more of the compounds of Table 1-A, or a pharmaceutically acceptable salt or solvate thereof

TABLE 1

| Cpd. | | |
|---|---|---|
| No. | Structure | Chemical Name |
| 1 | 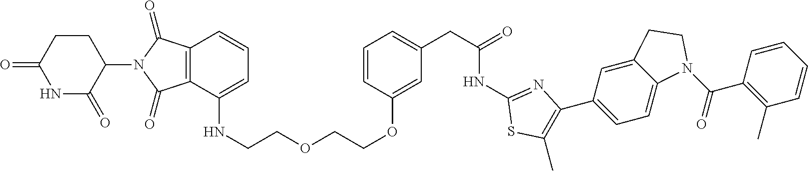 [SMILES: Cc1ccccc1C(=O)N1CCc2cc(-c3nc(NC(=O)Cc4cccc(OCCOCCNc5cccc6c5C(=O)N(C5CCC(=O)NC5=O)C6=O)c4)sc3C)ccc21] | 2-(3-(2-(2-((2-(2,6-dioxopiperidin-3- yl)-1,3-dioxoisoindolin-4- yl)amino)ethoxy)ethoxy)phenyl)-N-(5- methyl-4-(1-(2-methylbenzoyl)indolin- 5-yl)thiazol-2-yl)acetamide |
| 2 | 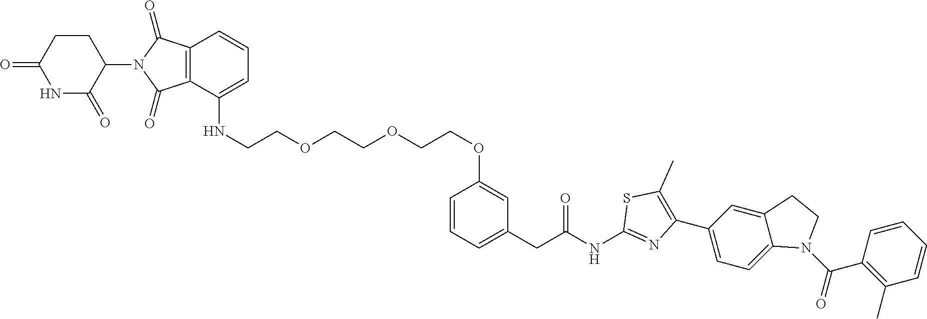 [SMILES: Cc1ccccc1C(=O)N1CCc2cc(-c3nc(NC(=O)Cc4cccc(OCCOCCOCCNc5cccc6c5C(=O)N(C5CCC(=O)NC5=O)C6=O)c4)sc3C)ccc21] | 2-(3-(2-(2-(2-((2-(2,6-dioxopiperidin- 3-yl)-1,3-dioxoisoindolin-4- yl)amino)ethoxy)ethoxy)ethoxy)phenyl)- N-(5-methyl-4-(1-(2- methylbenzoyl)indolin-5-yl)thiazol-2- yl)acetamide |
| 3 | 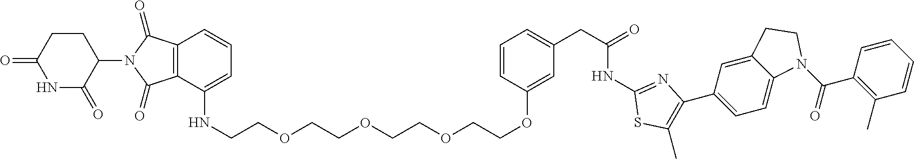 [SMILES: Cc1ccccc1C(=O)N1CCc2cc(-c3nc(NC(=O)Cc4cccc(OCCOCCOCCOCCNc5cccc6c5C(=O)N(C5CCC(=O)NC5=O)C6=O)c4)sc3C)ccc21] | 2-(3-(2-(2-(2-(2-((2-(2,6- dioxopiperidin-3-yl)-1,3- dioxoisoindolin-4- yl)amino)ethoxy)ethoxy)ethoxy)ethoxy) phenyl)-N-(5-methyl-4-(1-(2- methylbenzoyl)indolin-5-yl)thiazol-2- yl)acetamide |
| 4 | 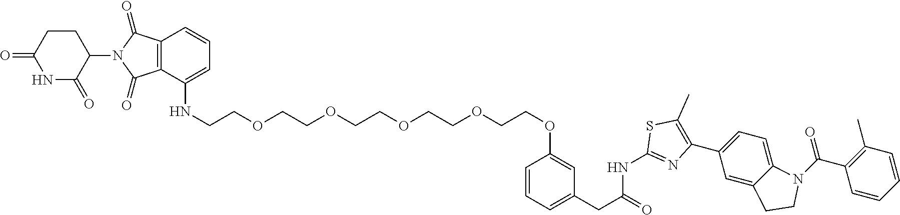 [SMILES: Cc1ccccc1C(=O)N1CCc2cc(-c3nc(NC(=O)Cc4cccc(OCCOCCOCCOCCOCCNc5cccc6c5C(=O)N(C5CCC(=O)NC5=O)C6=O)c4)sc3C)ccc21] | 2-(3-((14-((2-(2,6-dioxopiperidin-3- yl)-1,3-dioxoisoindolin-4-yl)amino)- 3,6,9,12- tetraoxatetradecyl)oxy)phenyl)-N-(5- methyl-4-(1-(2-methylbenzoyl)indolin- 5-yl)thiazol-2-yl)acetamide |
| 5 | 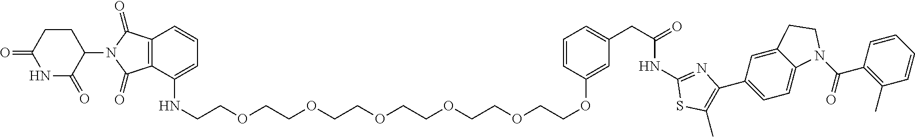 [SMILES: Cc1ccccc1C(=O)N1CCc2cc(-c3nc(NC(=O)Cc4cccc(OCCOCCOCCOCCOCCOCCNc5cccc6c5C(=O)N(C5CCC(=O)NC5=O)C6=O)c4)sc3C)ccc21] | 2-(3-((17-((2-(2,6-dioxopiperidin-3- yl)-1,3-dioxoisoindolin-4-yl)amino)- 3,6,9,12,15- pentaoxaheptadecyl)oxy)phenyl)-N-(5- methyl-4-(1-(2-methylbenzoyl)indolin- 5-yl)thiazol-2-yl)acetamide |
| 6 | 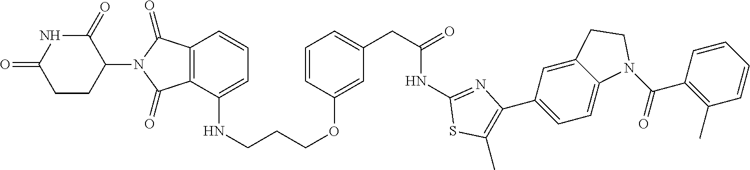 [SMILES: Cc1ccccc1C(=O)N1CCc2cc(-c3nc(NC(=O)Cc4cccc(OCCCNc5cccc6c5C(=O)N(C5CCC(=O)NC5=O)C6=O)c4)sc3C)ccc21] | 2-(3-(3-((2-(2,6-dioxopiperidin-3-yl)- 1,3-dioxoisoindolin-4- yl)amino)propoxy)phenyl)-N-(5- methyl-4-(1-(2-methylbenzoyl)indolin- 5-yl)thiazol-2-yl)acetamide |
| 7 | 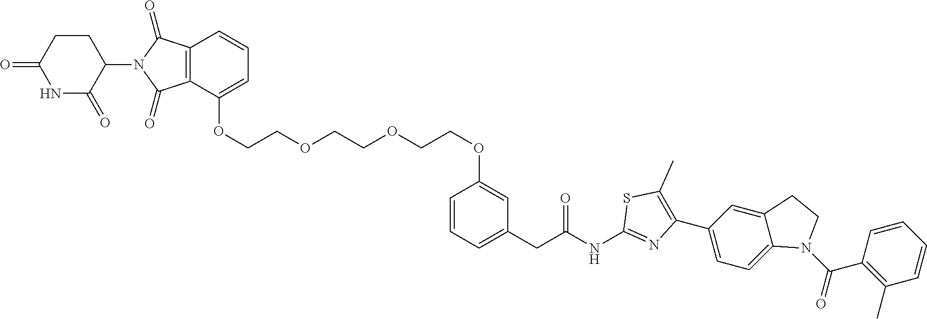 [SMILES: Cc1ccccc1C(=O)N1CCc2cc(-c3nc(NC(=O)Cc4cccc(OCCOCCOCCOc5cccc6c5C(=O)N(C5CCC(=O)NC5=O)C6=O)c4)sc3C)ccc21] | 2-(3-(2-(2-(2-((2-(2,6-dioxopiperidin- 3-yl)-1,3-dioxoisoindolin-4- yl)oxy)ethoxy)ethoxy)ethoxy)phenyl)- N-(5-methyl-4-(1-(2- methylbenzoyl)indolin-5-yl)thiazol-2- yl)acetamide |
| 8 | 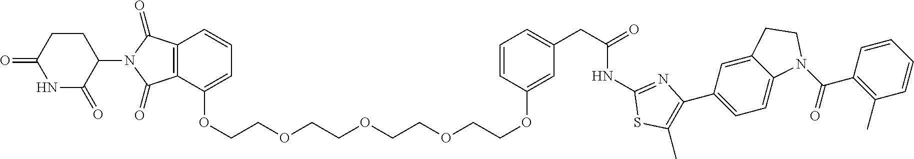 [SMILES: Cc1ccccc1C(=O)N1CCc2cc(-c3nc(NC(=O)Cc4cccc(OCCOCCOCCOCCOc5cccc6c5C(=O)N(C5CCC(=O)NC5=O)C6=O)c4)sc3C)ccc21] | 2-(3-(2-(2-(2-(2-((2-(2,6- dioxopiperidin-3-yl)-1,3- dioxoisoindolin-4- yl)oxy)ethoxy)ethoxy)ethoxy)ethoxy) phenyl)-N-(5-methyl-4-(1-(2- methylbenzoyl)indolin-5-yl)thiazol-2- yl)acetamide |
| 9 | 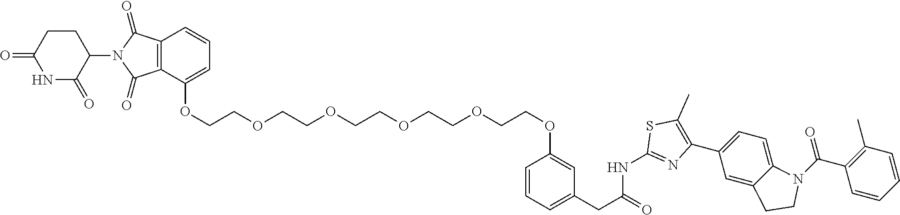 [SMILES: Cc1ccccc1C(=O)N1CCc2cc(-c3nc(NC(=O)Cc4cccc(OCCOCCOCCOCCOCCOc5cccc6c5C(=O)N(C5CCC(=O)NC5=O)C6=O)c4)sc3C)ccc21] | 2-(3-((14-((2-(2,6-dioxopiperidin-3- yl)-1,3-dioxoisoindolin-4-yl)oxy)- 3,6,9,12- tetraoxatetradecyl)oxy)phenyl)-N-(5- methyl-4-(1-(2-methylbenzoyl)indolin- 5-yl)thiazol-2-yl)acetamide |
| 10 | 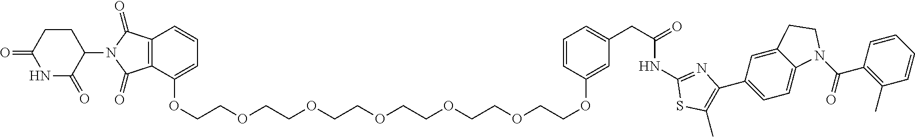 [SMILES: Cc1ccccc1C(=O)N1CCc2cc(-c3nc(NC(=O)Cc4cccc(OCCOCCOCCOCCOCCOCCOc5cccc6c5C(=O)N(C5CCC(=O)NC5=O)C6=O)c4)sc3C)ccc21] | 2-(3-((17-((2-(2,6-dioxopiperidin-3- yl)-1,3-dioxoisoindolin-4-yl)oxy)- 3,6,9,12,15- pentaoxaheptadecyl)oxy)phenyl)-N-(5- methyl-4-(1-(2-methylbenzoyl)indolin- 5-yl)thiazol-2-yl)acetamide |
| 11 | 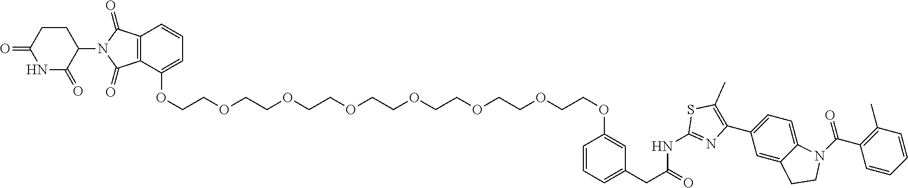 [SMILES: Cc1ccccc1C(=O)N1CCc2cc(-c3nc(NC(=O)Cc4cccc(OCCOCCOCCOCCOCCOCCOCCOc5cccc6c5C(=O)N(C5CCC(=O)NC5=O)C6=O)c4)sc3C)ccc21] | 2-(3-((20-((2-(2,6-dioxopiperidin-3- yl)-1,3-dioxoisoindolin-4-yl)oxy)- 3,6,9,12,15,18- hexaoxaicosyl)oxy)phenyl)-N-(5- methyl-4-(1-(2-methylbenzoyl)indolin- 5-yl)thiazol-2-yl)acetamide |
| 12 | 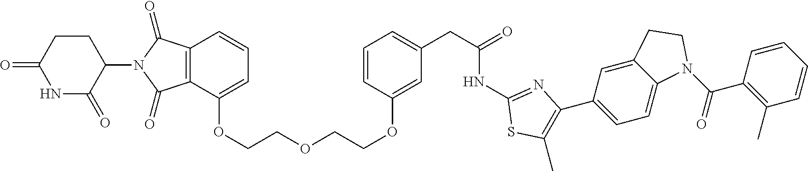 [SMILES: Cc1ccccc1C(=O)N1CCc2cc(-c3nc(NC(=O)Cc4cccc(OCCOCCOc5cccc6c5C(=O)N(C5CCC(=O)NC5=O)C6=O)c4)sc3C)ccc21] | 2-(3-(2-(2-((2-(2,6-dioxopiperidin-3- yl)-1,3-dioxoisoindolin-4- yl)oxy)ethoxy)ethoxy)phenyl)-N-(5- methyl-4-(1-(2-methylbenzoyl)indolin- 5-yl)thiazol-2-yl)acetamide |
| 13 | 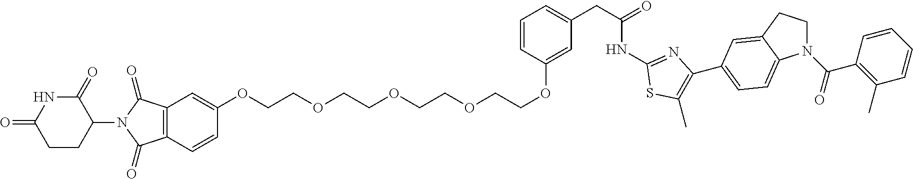 [SMILES: Cc1ccccc1C(=O)N1CCc2cc(-c3nc(NC(=O)Cc4cccc(OCCOCCOCCOCCOc5ccc6c(c5)C(=O)N(C5CCC(=O)NC5=O)C6=O)c4)sc3C)ccc21] | 2-(3-(2-(2-(2-(2-((2-(2,6- dioxopiperidin-3-yl)-1,3- dioxoisoindolin-5- yl)oxy)ethoxy)ethoxy)ethoxy)ethoxy) phenyl)-N-(5-methyl-4-(1-(2- methylbenzoyl)indolin-5-yl)thiazol-2- yl)acetamide |
| 14 | 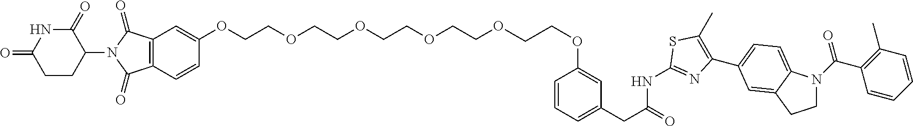 [SMILES: Cc1ccccc1C(=O)N1CCc2cc(-c3nc(NC(=O)Cc4cccc(OCCOCCOCCOCCOCCOc5ccc6c(c5)C(=O)N(C5CCC(=O)NC5=O)C6=O)c4)sc3C)ccc21] | 2-(3-((14-((2-(2,6-dioxopiperidin-3- yl)-1,3-dioxoisoindolin-5-yl)oxy)- 3,6,9,12- tetraoxatetradecyl)oxy)phenyl)-N-(5- methyl-4-(1-(2-methylbenzoyl)indolin- 5-yl)thiazol-2-yl)acetamide |
| 15 | 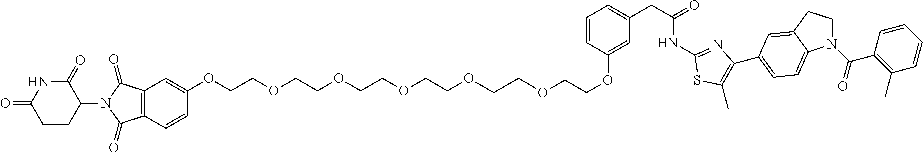 [SMILES: Cc1ccccc1C(=O)N1CCc2cc(-c3nc(NC(=O)Cc4cccc(OCCOCCOCCOCCOCCOCCOc5ccc6c(c5)C(=O)N(C5CCC(=O)NC5=O)C6=O)c4)sc3C)ccc21] | 2-(3-((17-((2-(2,6-dioxopiperidin-3- yl)-1,3-dioxoisoindolin-5-yl)oxy)- 3,6,9,12,15- pentaoxaheptadecyl)oxy)phenyl)-N-(5- methyl-4-(1-(2-methylbenzoyl)indolin- 5-yl)thiazol-2-yl)acetamide |
| 16 | 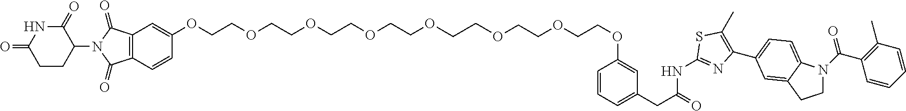 [SMILES: Cc1ccccc1C(=O)N1CCc2cc(-c3nc(NC(=O)Cc4cccc(OCCOCCOCCOCCOCCOCCOCCOc5ccc6c(c5)C(=O)N(C5CCC(=O)NC5=O)C6=O)c4)sc3C)ccc21] | 2-(3-((20-((2-(2,6-dioxopiperidin-3- yl)-1,3-dioxoisoindolin-5-yl)oxy)- 3,6,9,12,15,18- hexaoxaicosyl)oxy)phenyl)-N-(5- methyl-4-(1-(2-methylbenzoyl)indolin- 5-yl)thiazol-2-yl)acetamide |
| 17 | 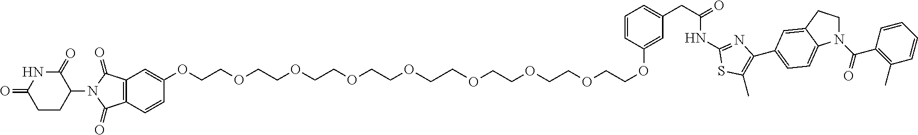 [SMILES: Cc1ccccc1C(=O)N1CCc2cc(-c3nc(NC(=O)Cc4cccc(OCCOCCOCCOCCOCCOCCOCCOCCOc5ccc6c(c5)C(=O)N(C5CCC(=O)NC5=O)C6=O)c4)sc3C)ccc21] | 2-(3-((23-((2-(2,6-dioxopiperidin-3- yl)-1,3-dioxoisoindolin-5-yl)oxy)- 3,6,9,12,15,18,21- heptaoxatricosyl)oxy)phenyl)-N-(5- methyl-4-(1-(2-methylbenzoyl)indolin- 5-yl)thiazol-2-yl)acetamide |
| 18 | 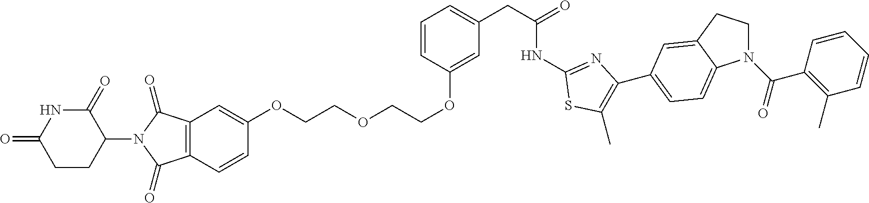 [SMILES: Cc1ccccc1C(=O)N1CCc2cc(-c3nc(NC(=O)Cc4cccc(OCCOCCOc5ccc6c(c5)C(=O)N(C5CCC(=O)NC5=O)C6=O)c4)sc3C)ccc21] | 2-(3-(2-(2-((2-(2,6-dioxopiperidin-3- yl)-1,3-dioxoisoindolin-5- yl)oxy)ethoxy)ethoxy)phenyl)-N-(5- methyl-4-(1-(2-methylbenzoyl)indolin- 5-yl)thiazol-2-yl)acetamide |
| 19 | 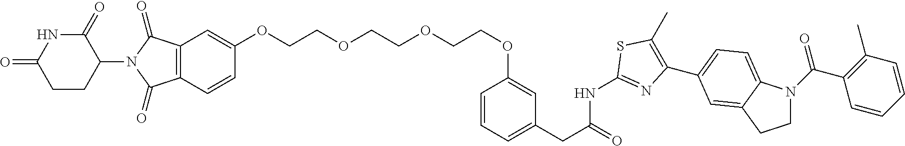 [SMILES: Cc1ccccc1C(=O)N1CCc2cc(-c3nc(NC(=O)Cc4cccc(OCCOCCOCCOc5ccc6c(c5)C(=O)N(C5CCC(=O)NC5=O)C6=O)c4)sc3C)ccc21] | 2-(3-(2-(2-(2-((2-(2,6-dioxopiperidin- 3-yl)-1,3-dioxoisoindolin-5- yl)oxy)ethoxy)ethoxy)ethoxy)phenyl)- N-(5-methyl-4-(1-(2- methylbenzoyl)indolin-5-yl)thiazol-2- yl)acetamide |
| 20 | 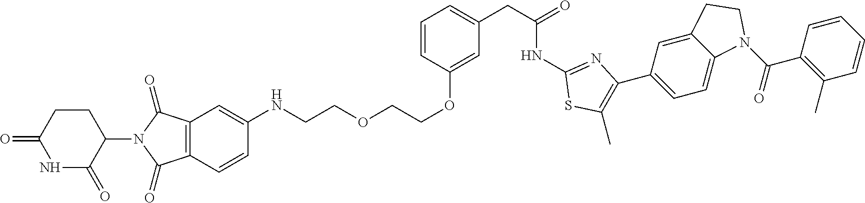 [SMILES: Cc1ccccc1C(=O)N1CCc2cc(-c3nc(NC(=O)Cc4cccc(OCCOCCNc5ccc6c(c5)C(=O)N(C5CCC(=O)NC5=O)C6=O)c4)sc3C)ccc21] | 2-(3-(2-(2-((2-(2,6-dioxopiperidin-3- yl)-1,3-dioxoisoindolin-5- yl)amino)ethoxy)ethoxy)phenyl)-N-(5- methyl-4-(1-(2-methylbenzoyl)indolin- 5-yl)thiazol-2-yl)acetamide |
| 21 | 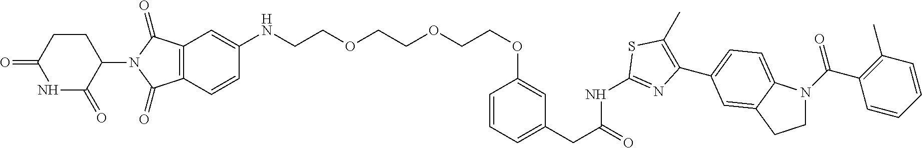 [SMILES: Cc1ccccc1C(=O)N1CCc2cc(-c3nc(NC(=O)Cc4cccc(OCCOCCOCCNc5ccc6c(c5)C(=O)N(C5CCC(=O)NC5=O)C6=O)c4)sc3C)ccc21] | 2-(3-(2-(2-(2-((2-(2,6-dioxopiperidin- 3-yl)-1,3-dioxoisoindolin-5- yl)amino)ethoxy)ethoxy)ethoxy)phenyl)- N-(5-methyl-4-(1-(2- methylbenzoyl)indolin-5-yl)thiazol-2- yl)acetamide |
| 22 | 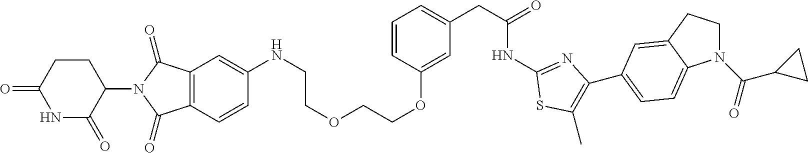 [SMILES: Cc1sc(NC(=O)Cc2cccc(OCCOCCNc3ccc4c(c3)C(=O)N(C3CCC(=O)NC3=O)C4=O)c2)nc1-c1ccc2c(c1)CCN2C(=O)C1CC1] | N-(4-(1- (cyclopropanecarbonyl)indolin-5-yl)- 5-methylthiazol-2-yl)-2-(3-(2-(2-((2- (2,6-dioxopiperidin-3-yl)-1,3- dioxoisoindolin-5- yl)amino)ethoxy)ethoxy)phenyl) acetamide |
| 23 | 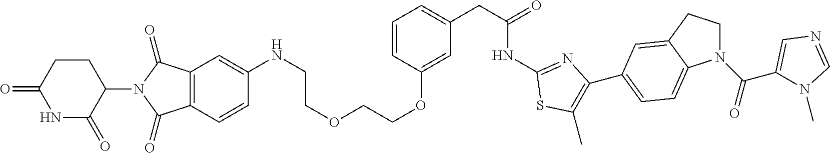 [SMILES: Cc1sc(NC(=O)Cc2cccc(OCCOCCNc3ccc4c(c3)C(=O)N(C3CCC(=O)NC3=O)C4=O)c2)nc1-c1ccc2c(c1)CCN2C(=O)c1cncn1C] | 2-(3-(2-(2-((2-(2,6-dioxopiperidin-3- yl)-1,3-dioxoisoindolin-5- yl)amino)ethoxy)ethoxy)phenyl)-N-(5- methyl-4-(1-(1-methyl-1H-imidazole- 5-carbonyl)indolin-5-yl)thiazol-2- yl)acetamide |
| 24 | 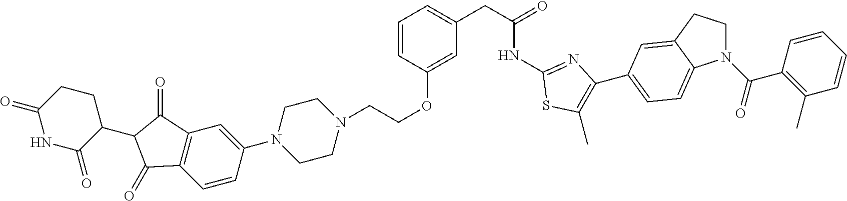 [SMILES: Cc1ccccc1C(=O)N1CCc2cc(-c3nc(NC(=O)Cc4cccc(OCCN5CCN(c6ccc7c(c6)C(=O)C(C6CCC(=O)NC6=O)C7=O)CC5)c4)sc3C)ccc21] | 2-(3-(2-(4-(2-(2,6-dioxopiperidin-3- yl)-1,3-dioxoisoindolin-5-yl)piperazin- 1-yl)ethoxy)phenyl)-N-(5-methyl-4-(1- (2-methylbenzoyl)indolin-5-yl)thiazol- 2-yl)acetamide |
| 25 | 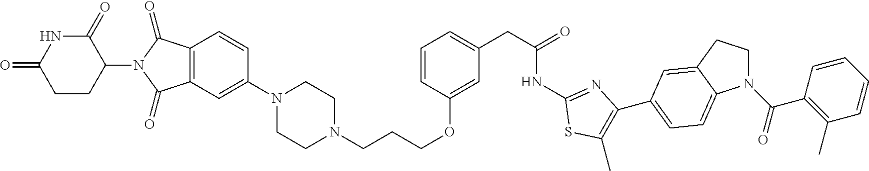 [SMILES: Cc1ccccc1C(=O)N1CCc2cc(-c3nc(NC(=O)Cc4cccc(OCCCN5CCN(c6ccc7c(c6)C(=O)N(C6CCC(=O)NC6=O)C7=O)CC5)c4)sc3C)ccc21] | 2-(3-(3-(4-(2-(2,6-dioxopiperidin-3- yl)-1,3-dioxoisoindolin-5-yl)piperazin- 1-yl)propoxy)phenyl)-N-(5-methyl-4- (1-(2-methylbenzoyl)indolin-5- yl)thiazol-2-yl)acetamide |
| 26 | 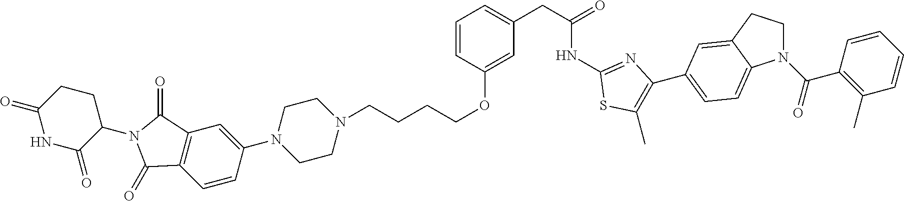 [SMILES: Cc1ccccc1C(=O)N1CCc2cc(-c3nc(NC(=O)Cc4cccc(OCCCCN5CCN(c6ccc7c(c6)C(=O)N(C6CCC(=O)NC6=O)C7=O)CC5)c4)sc3C)ccc21] | 2-(3-(4-(4-(2-(2,6-dioxopiperidin-3- yl)-1,3-dioxoisoindolin-5-yl)piperazin- 1-yl)butoxy)phenyl)-N-(5-methyl-4- (1-(2-methylbenzoyl)indolin-5- yl)thiazol-2-yl)acetamide |
| 27 | 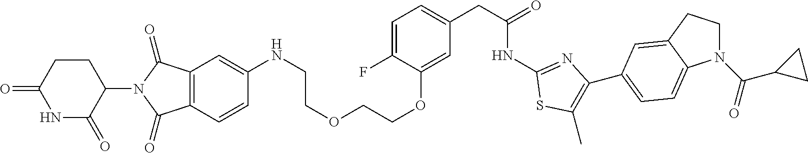 [SMILES: Cc1sc(NC(=O)Cc2ccc(F)c(OCCOCCNc3ccc4c(c3)C(=O)N(C3CCC(=O)NC3=O)C4=O)c2)nc1-c1ccc2c(c1)CCN2C(=O)C1CC1] | N-(4-(1- (cyclopropanecarbonyl)indolin-5-yl)- 5-methylthiazol-2-yl)-2-(3-(2-(2-((2- (2,6-dioxopiperidin-3-yl)-1,3- dioxoisoindolin-5- yl)amino)ethoxy)ethoxy)-4- fluorophenyl)acetamide |
| 28 | 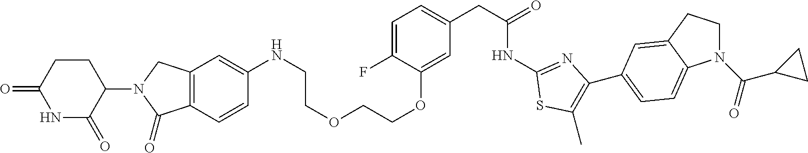 [SMILES: Cc1sc(NC(=O)Cc2ccc(F)c(OCCOCCNc3ccc4c(c3)CN(C3CCC(=O)NC3=O)C4=O)c2)nc1-c1ccc2c(c1)CCN2C(=O)C1CC1] | N-(4-(1- (cyclopropanecarbonyl)indolin-5-yl)- 5-methylthiazol-2-yl)-2-(3-(2-(2-((2- (2,6-dioxopiperidin-3-yl)-1- oxoisoindolin-5- yl)amino)ethoxy)ethoxy)-4- fluorophenyl)acetamide |
| 29 | 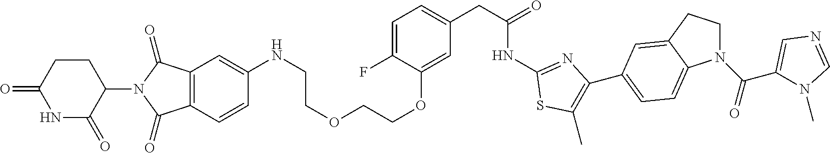 [SMILES: Cc1sc(NC(=O)Cc2ccc(F)c(OCCOCCNc3ccc4c(c3)C(=O)N(C3CCC(=O)NC3=O)C4=O)c2)nc1-c1ccc2c(c1)CCN2C(=O)c1cncn1C] | 2-(3-(2-(2-((2-(2,6-dioxopiperidin-3- yl)-1,3-dioxoisoindolin-5- yl)amino)ethoxy)ethoxy)-4- fluorophenyl)-N-(5-methyl-4-(1-(1- methyl-1H-imidazole-5- carbonyl)indolin-5-yl)thiazol-2- yl)acetamide |
| 30 | 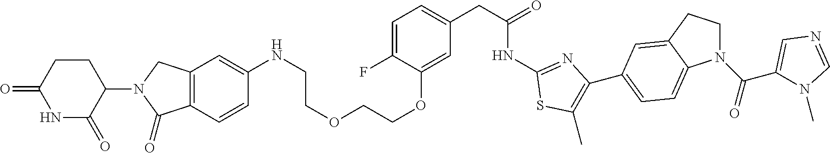 [SMILES: Cc1sc(NC(=O)Cc2ccc(F)c(OCCOCCNc3ccc4c(c3)CN(C3CCC(=O)NC3=O)C4=O)c2)nc1-c1ccc2c(c1)CCN2C(=O)c1cncn1C] | 2-(3-(2-(2-((2-(2,6-dioxopiperidin-3- yl)-1-oxoisoindolin-5- yl)amino)ethoxy)ethoxy)-4- fluorophenyl)-N-(5-methyl-4-(1-(1- methyl-1H-imidazole-5- carbonyl)indolin-5-yl)thiazol-2- yl)acetamide |
| 31 | 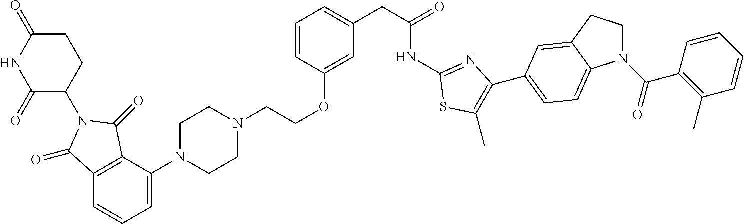 [SMILES: Cc1ccccc1C(=O)N1CCc2cc(-c3nc(NC(=O)Cc4cccc(OCCN5CCN(c6cccc7c6C(=O)N(C6CCC(=O)NC6=O)C7=O)CC5)c4)sc3C)ccc21] | 2-(3-(2-(4-(2-(2,6-dioxopiperidin-3- yl)-1,3-dioxoisoindolin-4-yl)piperazin- 1-yl)ethoxy)phenyl)-N-(5-methyl-4-(1- (2-methylbenzoyl)indolin-5-yl)thiazol- 2-yl)acetamide |
| 32 | 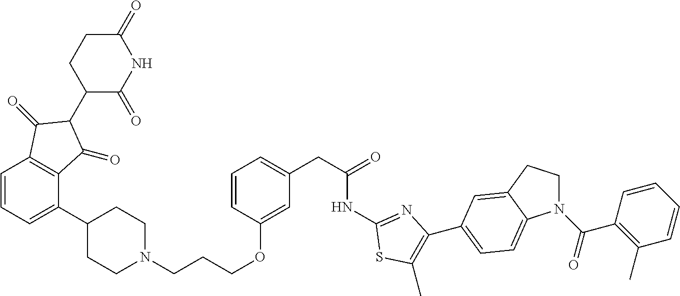 [SMILES: Cc1ccccc1C(=O)N1CCc2cc(-c3nc(NC(=O)Cc4cccc(OCCCN5CCC(c6cccc7c6C(=O)C(C6CCC(=O)NC6=O)C7=O)CC5)c4)sc3C)ccc21] | 2-(3-(3-(4-(2-(2,6-dioxopiperidin-3- yl)-1,3-dioxoisoindolin-4-yl)piperazin- 1-yl)propoxy)phenyl)-N-(5-methyl-4- (1-(2-methylbenzoyl)indolin-5- yl)thiazol-2-yl)acetamide |
| 33 | 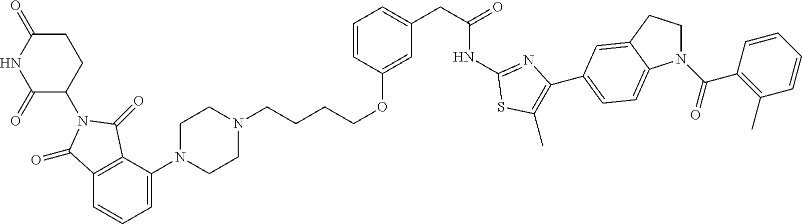 [SMILES: Cc1ccccc1C(=O)N1CCc2cc(-c3nc(NC(=O)Cc4cccc(OCCCCN5CCN(c6cccc7c6C(=O)N(C6CCC(=O)NC6=O)C7=O)CC5)c4)sc3C)ccc21] | 2-(3-(4-(4-(2-(2,6-dioxopiperidin-3- yl)-1,3-dioxoisoindolin-4-yl)piperazin- 1-yl)butoxy)phenyl)-N-(5-methyl-4- (1-(2-methylbenzoyl)indolin-5- yl)thiazol-2-yl)acetamide |
| 34 | 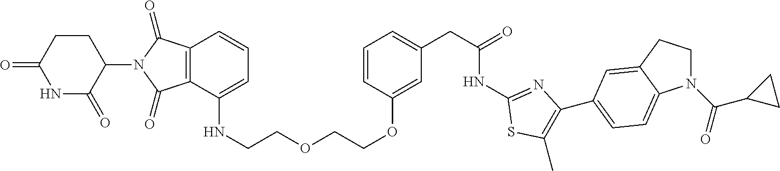 [SMILES: Cc1sc(NC(=O)Cc2cccc(OCCOCCNc3cccc4c3C(=O)N(C3CCC(=O)NC3=O)C4=O)c2)nc1-c1ccc2c(c1)CCN2C(=O)C1CC1] | N-(4-(1- (cyclopropanecarbonyl)indolin-5-yl)- 5-methylthiazol-2-yl)-2-(3-(2-(2-((2- (2,6-dioxopiperidin-3-yl)-1,3- dioxoisoindolin-4- yl)amino)ethoxy)ethoxy)phenyl) acetamide |
| 35 | 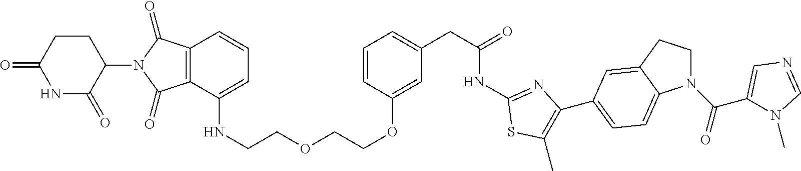 [SMILES: Cc1sc(NC(=O)Cc2cccc(OCCOCCNc3cccc4c3C(=O)N(C3CCC(=O)NC3=O)C4=O)c2)nc1-c1ccc2c(c1)CCN2C(=O)c1cncn1C] | 2-(3-(2-(2-((2-(2,6-dioxopiperidin-3- yl)-1,3-dioxoisoindolin-4- yl)amino)ethoxy)ethoxy)phenyl)-N-(5- methyl-4-(1-(1-methyl-1H-imidazole- 5-carbonyl)indolin-5-yl)thiazol-2- yl)acetamide |
| 36 | 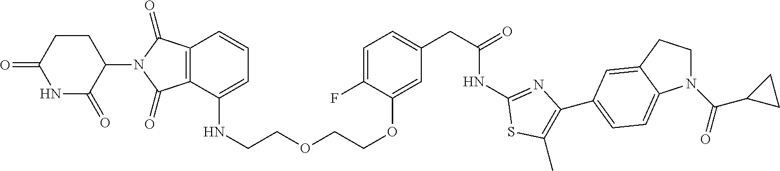 [SMILES: Cc1sc(NC(=O)Cc2ccc(F)c(OCCOCCNc3cccc4c3C(=O)N(C3CCC(=O)NC3=O)C4=O)c2)nc1-c1ccc2c(c1)CCN2C(=O)C1CC1] | N-(4-(1- (cyclopropanecarbonyl)indolin-5-yl)- 5-methylthiazol-2-yl)-2-(3-(2-(2-((2- (2,6-dioxopiperidin-3-yl)-1,3- dioxoisoindolin-4- yl)amino)ethoxy)ethoxy)-4- fluorophenyl)acetamide |
| 37 | 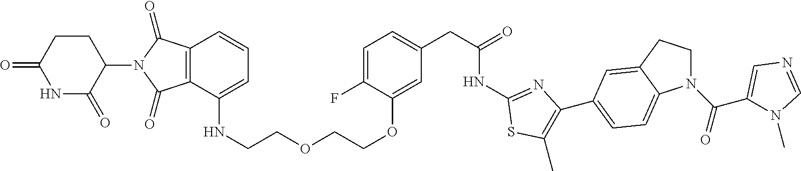 [SMILES: Cc1sc(NC(=O)Cc2ccc(F)c(OCCOCCNc3cccc4c3C(=O)N(C3CCC(=O)NC3=O)C4=O)c2)nc1-c1ccc2c(c1)CCN2C(=O)c1cncn1C] | 2-(3-(2-(2-((2-(2,6-dioxopiperidin-3- yl)-1,3-dioxoisoindolin-4- yl)amino)ethoxy)ethoxy)-4- fluorophenyl)-N-(5-methyl-4-(1-(1- methyl-1H-imidazole-5- carbonyl)indolin-5-yl)thiazol-2- yl)acetamide |
| 38 | 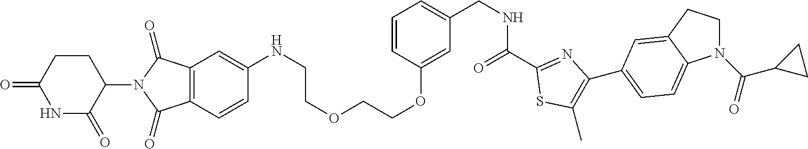 [SMILES: Cc1sc(C(=O)NCc2cccc(OCCOCCNc3ccc4c(c3)C(=O)N(C3CCC(=O)NC3=O)C4=O)c2)nc1-c1ccc2c(c1)CCN2C(=O)C1CC1] | 4-(1-(cyclopropanecarbonyl)indolin-5- yl)-N-(3-(2-(2-((2-(2,6-dioxopiperidin- 3-yl)-1,3-dioxoisoindolin-5- yl)amino)ethoxy)ethoxy)benzyl)-5- methylthiazole-2-carboxamide |
| 39 | 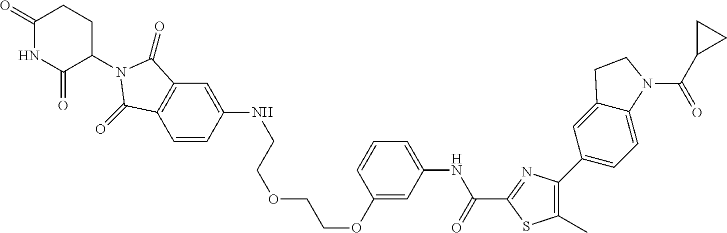 [SMILES: Cc1sc(C(=O)Nc2cccc(OCCOCCNc3ccc4c(c3)C(=O)N(C3CCC(=O)NC3=O)C4=O)c2)nc1-c1ccc2c(c1)CCN2C(=O)C1CC1] | 4-(1-(cyclopropanecarbonyl)indolin-5- yl)-N-(3-(2-(2-((2-(2,6-dioxopiperidin- 3-yl)-1,3-dioxoisoindolin-5- yl)amino)ethoxy)ethoxy)phenyl)-5- methylthiazole-2-carboxamide |
| 40 | 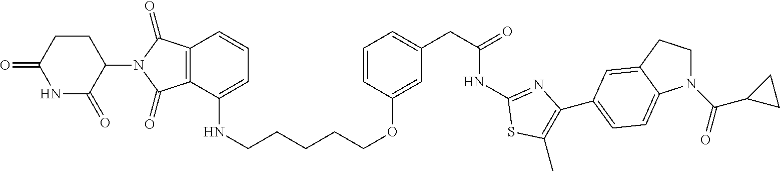 [SMILES: Cc1sc(NC(=O)Cc2cccc(OCCCCCNc3cccc4c3C(=O)N(C3CCC(=O)NC3=O)C4=O)c2)nc1-c1ccc2c(c1)CCN2C(=O)C1CC1] | N-(4-(1- (cyclopropanecarbonyl)indolin-5-yl)- 5-methylthiazol-2-yl)-2-(3-((5-((2- (2,6-dioxopiperidin-3-yl)-1,3- dioxoisoindolin-4- yl)amino)pentyl)oxy)phenyl)acetamide |
| 41 | 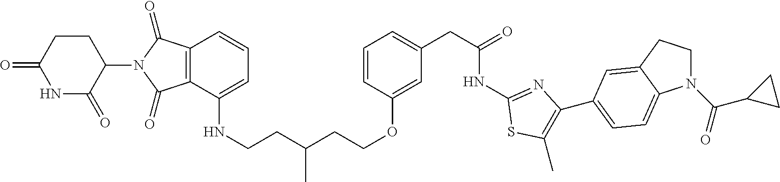 [SMILES: Cc1sc(NC(=O)Cc2cccc(OCCC(C)CCNc3cccc4c3C(=O)N(C3CCC(=O)NC3=O)C4=O)c2)nc1-c1ccc2c(c1)CCN2C(=O)C1CC1] | N-(4-(1- (cyclopropanecarbonyl)indolin-5-yl)- 5-methylthiazol-2-yl)-2-(3-((5-((2- (2,6-dioxopiperidin-3-yl)-1,3- dioxoisoindolin-4-yl)amino)-3- methylpentyl)oxy)phenyl)acetamide |
| 42 | 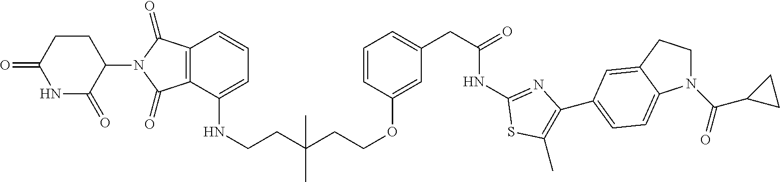 [SMILES: Cc1sc(NC(=O)Cc2cccc(OCCC(C)(C)CCNc3cccc4c3C(=O)N(C3CCC(=O)NC3=O)C4=O)c2)nc1-c1ccc2c(c1)CCN2C(=O)C1CC1] | N-(4-(1- (cyclopropanecarbonyl)indolin-5-yl)- 5-methylthiazol-2-yl)-2-(3-((5-((2- (2,6-dioxopiperidin-3-yl)-1,3- dioxoisoindolin-4-yl)amino)-3,3- dimethylpentyl)oxy)phenyl)acetamide |
| 43 | 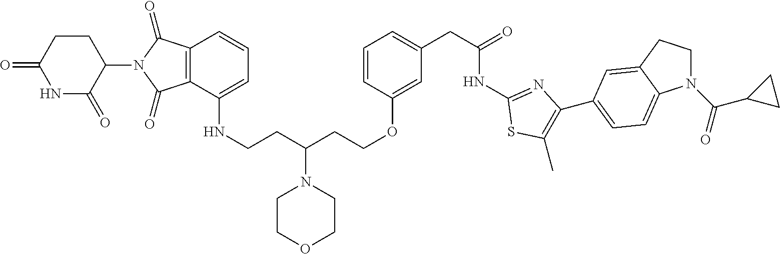 [SMILES: Cc1sc(NC(=O)Cc2cccc(OCCC(CCNc3cccc4c3C(=O)N(C3CCC(=O)NC3=O)C4=O)N3CCOCC3)c2)nc1-c1ccc2c(c1)CCN2C(=O)C1CC1] | N-(4-(1- (cyclopropanecarbonyl)indolin-5-yl)- 5-methylthiazol-2-yl)-2-(3-((5-((2- (2,6-dioxopiperidin-3-yl)-1,3- dioxoisoindolin-4-yl)amino)-3- morpholinopentyl)oxy)phenyl) acetamide |
| 44 | 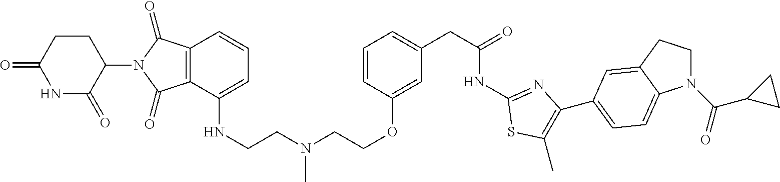 [SMILES: Cc1sc(NC(=O)Cc2cccc(OCCN(C)CCNc3cccc4c3C(=O)N(C3CCC(=O)NC3=O)C4=O)c2)nc1-c1ccc2c(c1)CCN2C(=O)C1CC1] | N-(4-(1- (cyclopropanecarbonyl)indolin-5-yl)- 5-methylthiazol-2-yl)-2-(3-(2-((2-((2- (2,6-dioxopiperidin-3-yl)-1,3- dioxoisoindolin-4- yl)amino)ethyl)(methyl)amino)ethoxy) phenyl)acetamide |
| 45 | 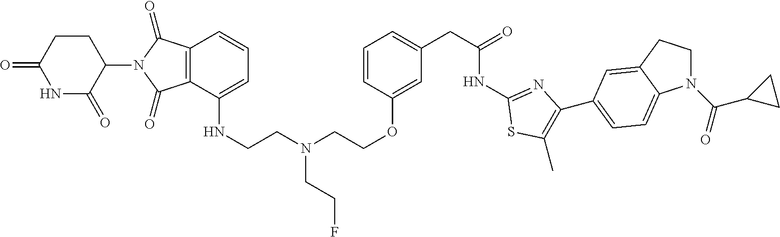 [SMILES: Cc1sc(NC(=O)Cc2cccc(OCCN(CCF)CCNc3cccc4c3C(=O)N(C3CCC(=O)NC3=O)C4=O)c2)nc1-c1ccc2c(c1)CCN2C(=O)C1CC1] | N-(4-(1- (cyclopropanecarbonyl)indolin-5-yl)- 5-methylthiazol-2-yl)-2-(3-(2-((2-((2- (2,6-dioxopiperidin-3-yl)-1,3- dioxoisoindolin-4-yl)amino)ethyl)(2- fluoroethyl)amino)ethoxy)phenyl) acetamide |
| 46 | 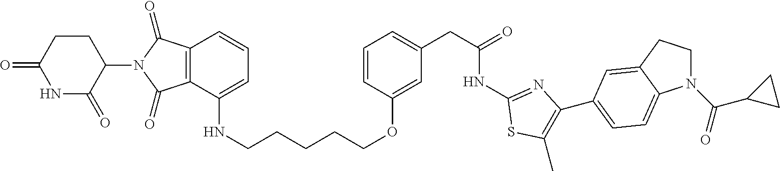 [SMILES: Cc1sc(NC(=O)Cc2cccc(OCCCCCNc3cccc4c3C(=O)N(C3CCC(=O)NC3=O)C4=O)c2)nc1-c1ccc2c(c1)CCN2C(=O)C1CC1] | N-(4-(1- (cyclopropanecarbonyl)indolin-5-yl)- 5-methylthiazol-2-yl)-2-(3-((5-((2- (2,6-dioxopiperidin-3-yl)-1,3- dioxoisoindolin-4- yl)oxy)pentyl)oxy)phenyl)acetamide |
| 47 | 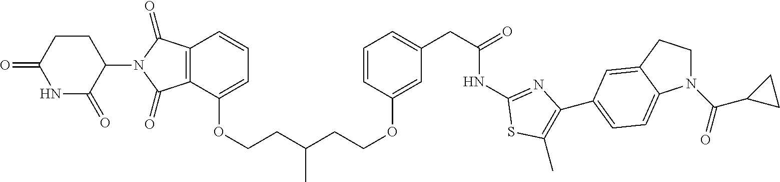 [SMILES: Cc1sc(NC(=O)Cc2cccc(OCCC(C)CCOc3cccc4c3C(=O)N(C3CCC(=O)NC3=O)C4=O)c2)nc1-c1ccc2c(c1)CCN2C(=O)C1CC1] | N-(4-(1- (cyclopropanecarbonyl)indolin-5-yl)- 5-methylthiazol-2-yl)-2-(3-((5-((2- (2,6-dioxopiperidin-3-yl)-1,3- dioxoisoindolin-4-yl)oxy)-3- methylpentyl)oxy)phenyl)acetamide |
| 48 | 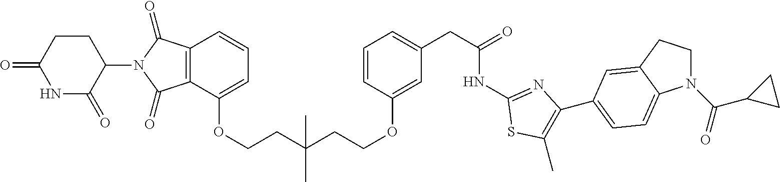 [SMILES: Cc1sc(NC(=O)Cc2cccc(OCCC(C)(C)CCOc3cccc4c3C(=O)N(C3CCC(=O)NC3=O)C4=O)c2)nc1-c1ccc2c(c1)CCN2C(=O)C1CC1] | N-(4-(1- (cyclopropanecarbonyl)indolin-5-yl)- 5-methylthiazol-2-yl)-2-(3-((5-((2- (2,6-dioxopiperidin-3-yl)-1,3- dioxoisoindolin-4-yl)oxy)-3,3- dimethylpentyl)oxy)phenyl)acetamide |
| 49 | 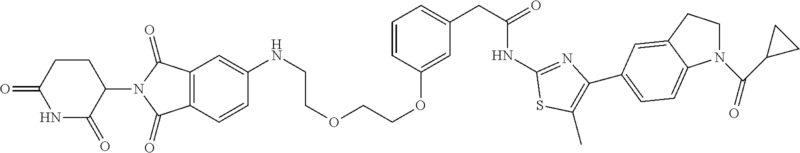 [SMILES: Cc1sc(NC(=O)Cc2cccc(OCCOCCNc3ccc4c(c3)C(=O)N(C3CCC(=O)NC3=O)C4=O)c2)nc1-c1ccc2c(c1)CCN2C(=O)C1CC1] | N-(4-(1- (cyclopropanecarbonyl)indolin-5-yl)- 5-methylthiazol-2-yl)-2-(3-((5-((2- (2,6-dioxopiperidin-3-yl)-1,3- dioxoisoindolin-5- yl)amino)pentyl)oxy)phenyl)acetamide |
| 50 | 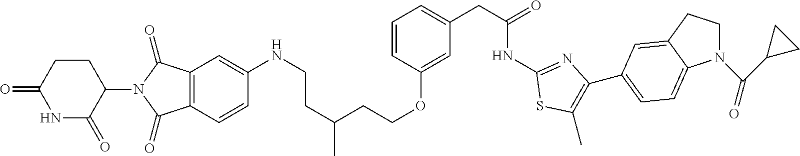 [SMILES: Cc1sc(NC(=O)Cc2cccc(OCCC(C)CCNc3ccc4c(c3)C(=O)N(C3CCC(=O)NC3=O)C4=O)c2)nc1-c1ccc2c(c1)CCN2C(=O)C1CC1] | N-(4-(1- (cyclopropanecarbonyl)indolin-5-yl)- 5-methylthiazol-2-yl)-2-(3-((5-((2- (2,6-dioxopiperidin-3-yl)-1,3- dioxoisoindolin-5-yl)amino)-3- methylpentyl)oxy)phenyl)acetamide |
| 51 | 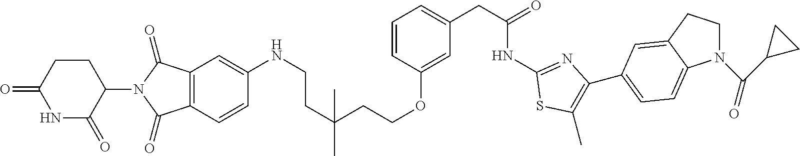 [SMILES: Cc1sc(NC(=O)Cc2cccc(OCCC(C)(C)CCNc3ccc4c(c3)C(=O)N(C3CCC(=O)NC3=O)C4=O)c2)nc1-c1ccc2c(c1)CCN2C(=O)C1CC1] | N-(4-(1- (cyclopropanecarbonyl)indolin-5-yl)- 5-methylthiazol-2-yl)-2-(3-((5-((2- (2,6-dioxopiperidin-3-yl)-1,3- dioxoisoindolin-5-yl)amino)-3,3- dimethylpentyl)oxy)phenyl)acetamide |
| 52 | 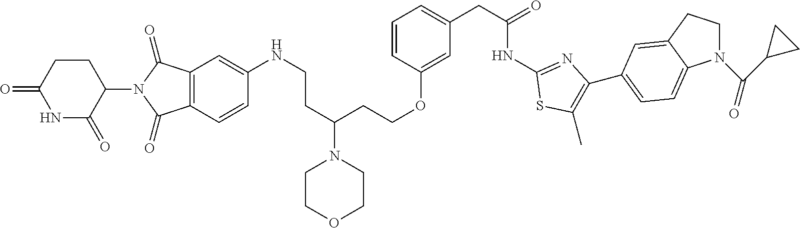 [SMILES: Cc1sc(NC(=O)Cc2cccc(OCCC(CCNc3ccc4c(c3)C(=O)N(C3CCC(=O)NC3=O)C4=O)N3CCOCC3)c2)nc1-c1ccc2c(c1)CCN2C(=O)C1CC1] | N-(4-(1- (cyclopropanecarbonyl)indolin-5-yl)- 5-methylthiazol-2-yl)-2-(3-((5-((2- (2,6-dioxopiperidin-3-yl)-1,3- dioxoisoindolin-5-yl)amino)-3- morpholinopentyl)oxy)phenyl) acetamide |
| 53 | 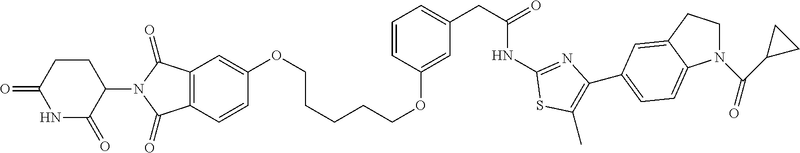 [SMILES: Cc1sc(NC(=O)Cc2cccc(OCCCCCOc3ccc4c(c3)C(=O)N(C3CCC(=O)NC3=O)C4=O)c2)nc1-c1ccc2c(c1)CCN2C(=O)C1CC1] | N-(4-(1- (cyclopropanecarbonyl)indolin-5-yl)- 5-methylthiazol-2-yl)-2-(3-((5-((2- (2,6-dioxopiperidin-3-yl)-1,3- dioxoisoindolin-5- yl)oxy)pentyl)oxy)phenyl)acetamide |
| 54 | 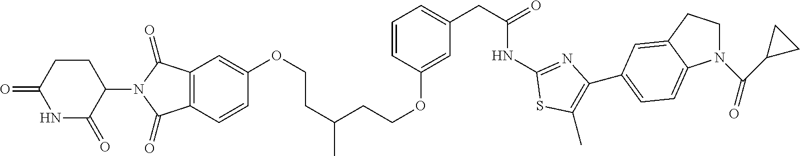 [SMILES: Cc1sc(NC(=O)Cc2cccc(OCCC(C)CCOc3ccc4c(c3)C(=O)N(C3CCC(=O)NC3=O)C4=O)c2)nc1-c1ccc2c(c1)CCN2C(=O)C1CC1] | N-(4-(1- (cyclopropanecarbonyl)indolin-5-yl)- 5-methylthiazol-2-yl)-2-(3-((5-((2- (2,6-dioxopiperidin-3-yl)-1,3- dioxoisoindolin-5-yl)oxy)-3- methylpentyl)oxy)phenyl)acetamide |
| 55 | 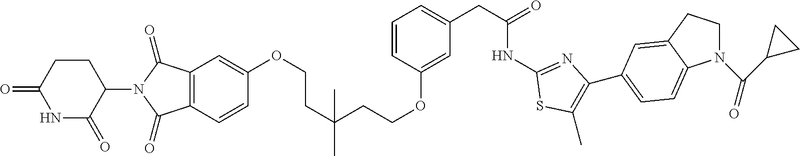 [SMILES: Cc1sc(NC(=O)Cc2cccc(OCCC(C)(C)CCOc3ccc4c(c3)C(=O)N(C3CCC(=O)NC3=O)C4=O)c2)nc1-c1ccc2c(c1)CCN2C(=O)C1CC1] | N-(4-(1- (cyclopropanecarbonyl)indolin-5-yl)- 5-methylthiazol-2-yl)-2-(3-((5-((2- (2,6-dioxopiperidin-3-yl)-1,3- dioxoisoindolin-5-yl)oxy)-3,3- dimethylpentyl)oxy)phenyl)acetamide |
| 56 | 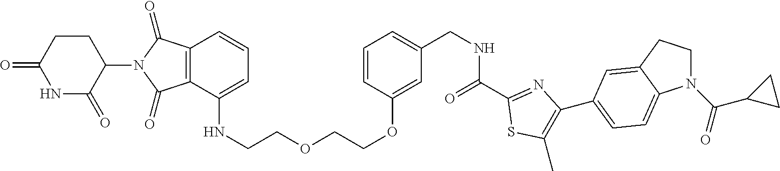 [SMILES: Cc1sc(C(=O)NCc2cccc(OCCOCCNc3cccc4c3C(=O)N(C3CCC(=O)NC3=O)C4=O)c2)nc1-c1ccc2c(c1)CCN2C(=O)C1CC1] | 4-(1-(cyclopropanecarbonyl)indolin-5- yl)-N-(3-(2-(2-((2-(2,6-dioxopiperidin- 3-yl)-1,3-dioxoisoindolin-4- yl)amino)ethoxy)ethoxy)benzyl)-5- methylthiazole-2-carboxamide |
| 57 | 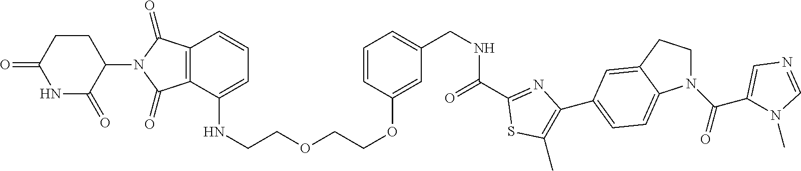 [SMILES: Cc1sc(C(=O)NCc2cccc(OCCOCCNc3cccc4c3C(=O)N(C3CCC(=O)NC3=O)C4=O)c2)nc1-c1ccc2c(c1)CCN2C(=O)c1cncn1C] | N-(3-(2-(2-((2-(2,6-dioxopiperidin-3- yl)-1,3-dioxoisoindolin-4- yl)amino)ethoxy)ethoxy)benzyl)-5- methyl-4-(1-(1-methyl-1H-imidazole- 5-carbonyl)indolin-5-yl)thiazole-2- carboxamide |
| 58 | 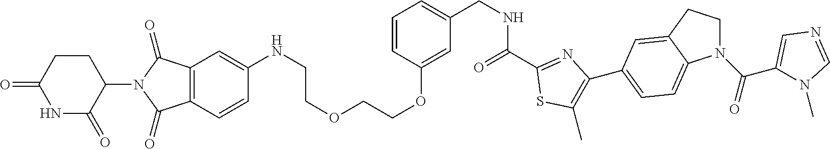 [SMILES: Cc1sc(C(=O)NCc2cccc(OCCOCCNc3ccc4c(c3)C(=O)N(C3CCC(=O)NC3=O)C4=O)c2)nc1-c1ccc2c(c1)CCN2C(=O)c1cncn1C] | N-(3-(2-(2-((2-(2,6-dioxopiperidin-3- yl)-1,3-dioxoisoindolin-5- yl)amino)ethoxy)ethoxy)benzyl)-5- methyl-4-(1-(1-methyl-1H-imidazole- 5-carbonyl)indolin-5-yl)thiazole-2- carboxamide |
| 59 | 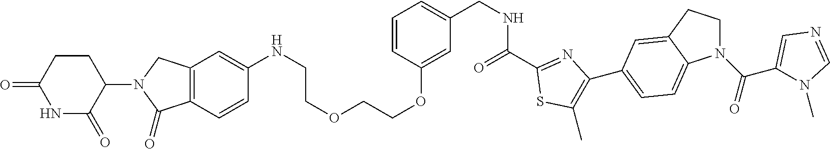 [SMILES: Cc1sc(C(=O)NCc2cccc(OCCOCCNc3ccc4c(c3)CN(C3CCC(=O)NC3=O)C4=O)c2)nc1-c1ccc2c(c1)CCN2C(=O)c1cncn1C] | N-(3-(2-(2-((2-(2,6-dioxopiperidin-3- yl)-1-oxoisoindolin-5- yl)amino)ethoxy)ethoxy)benzyl)-5- methyl-4-(1-(1-methyl-1H-imidazole- 5-carbonyl)indolin-5-yl)thiazole-2- carboxamide |
| 65 | 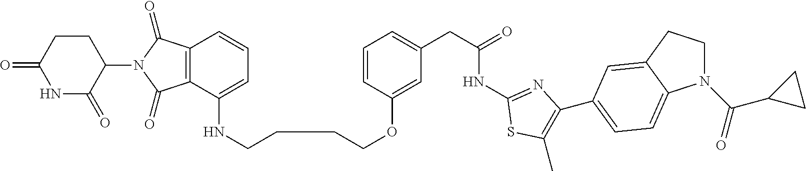 [SMILES: Cc1sc(NC(=O)Cc2cccc(OCCCCNc3cccc4c3C(=O)N(C3CCC(=O)NC3=O)C4=O)c2)nc1-c1ccc2c(c1)CCN2C(=O)C1CC1] | N-(4-(1- (cyclopropanecarbonyl)indolin-5-yl)- 5-methylthiazol-2-yl)-2-(3-(4-((2-(2,6- dioxopiperidin-3-yl)-1,3- dioxoisoindolin-4- yl)amino)butoxy)phenyl)acetamide |
| 66 | 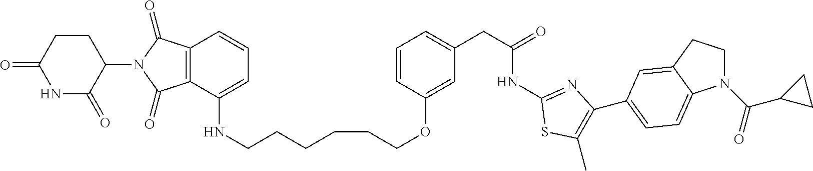 [SMILES: Cc1sc(NC(=O)Cc2cccc(OCCCCCCNc3cccc4c3C(=O)N(C3CCC(=O)NC3=O)C4=O)c2)nc1-c1ccc2c(c1)CCN2C(=O)C1CC1] | N-(4-(1- (cyclopropanecarbonyl)indolin-5-yl)- 5-methylthiazol-2-yl)-2-(3-((6-((2- (2,6-dioxopiperidin-3-yl)-1,3- dioxoisoindolin-4- yl)amino)hexyl)oxy)phenyl)acetamide |
| 67 | 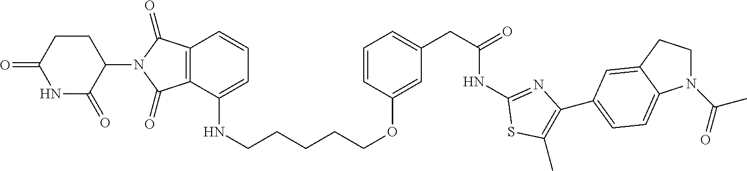 [SMILES: CC(=O)N1CCc2cc(-c3nc(NC(=O)Cc4cccc(OCCCCCNc5cccc6c5C(=O)N(C5CCC(=O)NC5=O)C6=O)c4)sc3C)ccc21] | N-(4-(1-acetylindolin-5-yl)-5- methylthiazol-2-yl)-2-(3-((5-((2-(2,6- dioxopiperidin-3-yl)-1,3- dioxoisoindolin-4- yl)amino)pentyl)oxy)phenyl)acetamide |
| 68 | 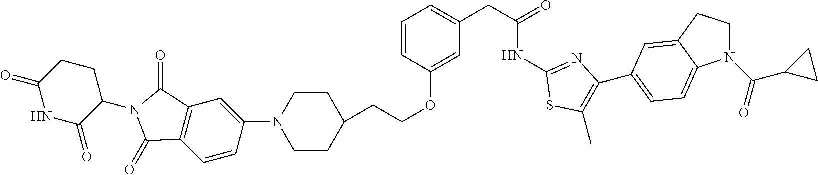 [SMILES: Cc1sc(NC(=O)Cc2cccc(OCCC3CCN(c4ccc5c(c4)C(=O)N(C4CCC(=O)NC4=O)C5=O)CC3)c2)nc1-c1ccc2c(c1)CCN2C(=O)C1CC1] | N-(4-(1- (cyclopropanecarbonyl)indolin-5-yl)- 5-methylthiazol-2-yl)-2-(3-(2-(1-(2- (2,6-dioxopiperidin-3-yl)-1,3- dioxoisoindolin-5-yl)piperidin-4- yl)ethoxy)phenyl)acetamide |
| 69 | 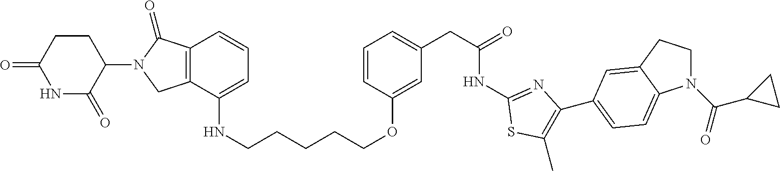 [SMILES: Cc1sc(NC(=O)Cc2cccc(OCCCCCNc3cccc4c3CN(C3CCC(=O)NC3=O)C4=O)c2)nc1-c1ccc2c(c1)CCN2C(=O)C1CC1] | N-(4-(1- (cyclopropanecarbonyl)indolin-5-yl)- 5-methylthiazol-2-yl)-2-(3-((5-((2- (2,6-dioxopiperidin-3-yl)-1- oxoisoindolin-4- yl)amino)pentyl)oxy)phenyl)acetamide |
| 70 | 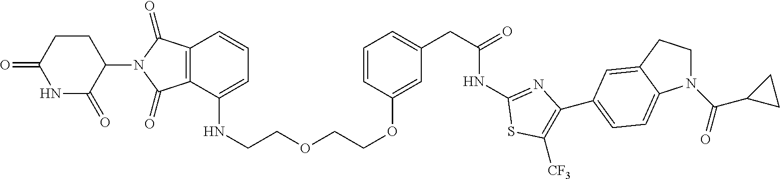 [SMILES: O=C1CCC(N2C(=O)c3cccc(NCCOCCOc4cccc(CC(=O)Nc5nc(-c6ccc7c(c6)CCN7C(=O)C6CC6)c(C(F)(F)F)s5)c4)c3C2=O)C(=O)N1] | N-(4-(1- (cyclopropanecarbonyl)indolin-5-yl)- 5-(trifluoromethyl)thiazol-2-yl)-2-(3- (2-(2-((2-(2,6-dioxopiperidin-3-yl)- 1,3-dioxoisoindolin-4- yl)amino)ethoxy)ethoxy)phenyl)aceta mide |
| 71 | 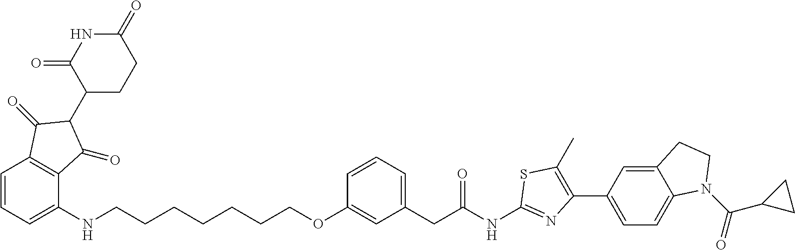 [SMILES: Cc1sc(NC(=O)Cc2cccc(OCCCCCCCNc3cccc4c3C(=O)C(C3CCC(=O)NC3=O)C4=O)c2)nc1-c1ccc2c(c1)CCN2C(=O)C1CC1] | N-(4-(1- (cyclopropanecarbonyl)indolin-5-yl)- 5-methylthiazol-2-yl)-2-(3-((7-((2- (2,6-dioxopiperidin-3-yl)-1,3- dioxoisoindolin-4- yl)amino)heptyl)oxy)phenyl)acetamide |
| 72 | 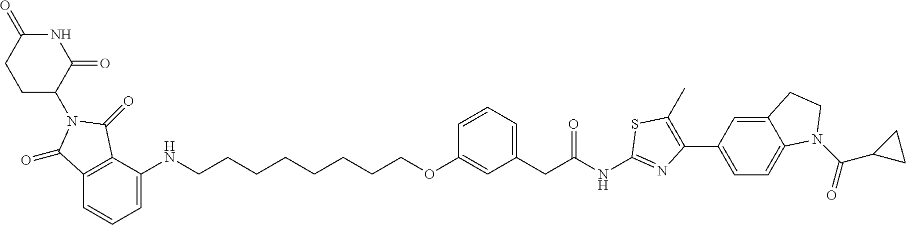 [SMILES: Cc1sc(NC(=O)Cc2cccc(OCCCCCCCCNc3cccc4c3C(=O)N(C3CCC(=O)NC3=O)C4=O)c2)nc1-c1ccc2c(c1)CCN2C(=O)C1CC1] | N-(4-(1- (cyclopropanecarbonyl)indolin-5-yl)- 5-methylthiazol-2-yl)-2-(3-((8-((2- (2,6-dioxopiperidin-3-yl)-1,3- dioxoisoindolin-4- yl)amino)octyl)oxy)phenyl)acetamide |
| 73 | 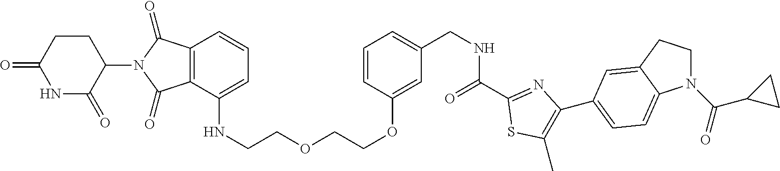 [SMILES: Cc1sc(C(=O)NCc2cccc(OCCOCCNc3cccc4c3C(=O)N(C3CCC(=O)NC3=O)C4=O)c2)nc1-c1ccc2c(c1)CCN2C(=O)C1CC1] | 4-(1-(cyclopropanecarbonyl)indolin-5- yl)-N-(3-((5-((2-(2,6-dioxopiperidin-3- yl)-1,3-dioxoisoindolin-4- yl)amino)pentyl)oxy)benzyl)-5- methylthiazole-2-carboxamide |
| 74 | 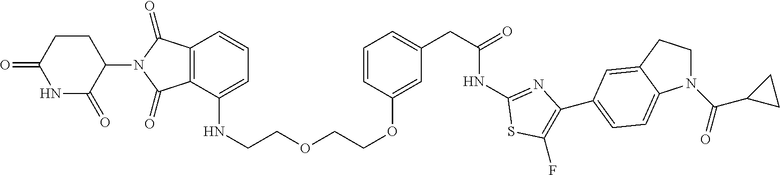 [SMILES: O=C1CCC(N2C(=O)c3cccc(NCCOCCOc4cccc(CC(=O)Nc5nc(-c6ccc7c(c6)CCN7C(=O)C6CC6)c(F)s5)c4)c3C2=O)C(=O)N1] | N-(4-(1- (cyclopropanecarbonyl)indolin-5-yl)- 5-fluorothiazol-2-yl)-2-(3-(2-(2-((2- (2,6-dioxopiperidin-3-yl)-1,3- dioxoisoindolin-4- yl)amino)ethoxy)ethoxy)phenyl) acetamide |
| 75 | 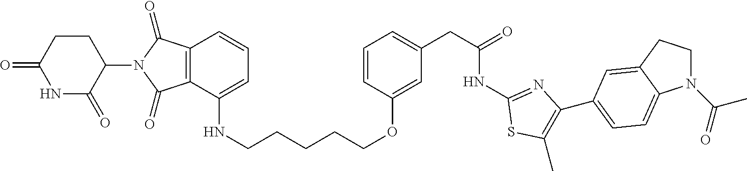 [SMILES: CC(=O)N1CCc2cc(-c3nc(NC(=O)Cc4cccc(OCCCCCNc5cccc6c5C(=O)N(C5CCC(=O)NC5=O)C6=O)c4)sc3C)ccc21] | N-(4-(1-acetyl-7-fluoroindolin-5-yl)-5- methylthiazol-2-yl)-2-(3-((5-((2-(2,6- dioxopiperidin-3-yl)-1,3- dioxoisoindolin-4- yl)amino)pentyl)oxy)phenyl)acetamide |
| 76 | 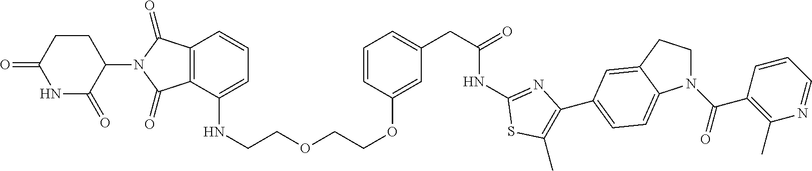 [SMILES: Cc1ncccc1C(=O)N1CCc2cc(-c3nc(NC(=O)Cc4cccc(OCCOCCNc5cccc6c5C(=O)N(C5CCC(=O)NC5=O)C6=O)c4)sc3C)ccc21] | 2-(3-(2-(2-((2-(2,6-dioxopiperidin-3- yl)-1,3-dioxoisoindolin-4- yl)amino)ethoxy)ethoxy)phenyl)-N-(5- methyl-4-(1-(2- methylnicotinoyl)indolin-5-yl)thiazol- 2-yl)acetamide |
| 77 | 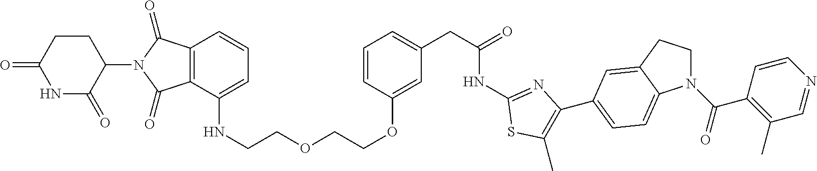 [SMILES: Cc1cnccc1C(=O)N1CCc2cc(-c3nc(NC(=O)Cc4cccc(OCCOCCNc5cccc6c5C(=O)N(C5CCC(=O)NC5=O)C6=O)c4)sc3C)ccc21] | 2-(3-(2-(2-((2-(2,6-dioxopiperidin-3- yl)-1,3-dioxoisoindolin-4- yl)amino)ethoxy)ethoxy)phenyl)-N-(5- methyl-4-(1-(3- methylisonicotinoyl)indolin-5- yl)thiazol-2-yl)acetamide |
| 78 | 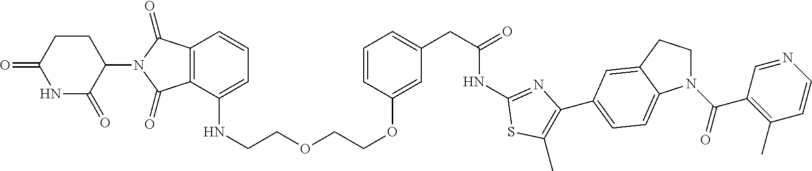 [SMILES: Cc1ccncc1C(=O)N1CCc2cc(-c3nc(NC(=O)Cc4cccc(OCCOCCNc5cccc6c5C(=O)N(C5CCC(=O)NC5=O)C6=O)c4)sc3C)ccc21] | 2-(3-(2-(2-((2-(2,6-dioxopiperidin-3- yl)-1,3-dioxoisoindolin-4- yl)amino)ethoxy)ethoxy)phenyl)-N-(5- methyl-4-(1-(4- methylnicotinoyl)indolin-5-yl)thiazol- 2-yl)acetamide |
| 79 | 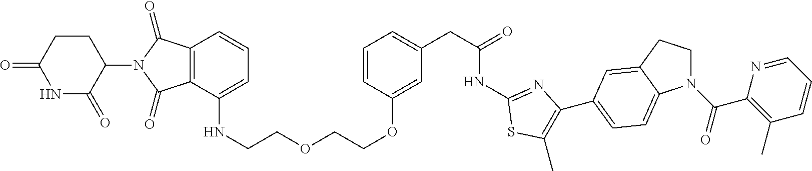 [SMILES: Cc1cccnc1C(=O)N1CCc2cc(-c3nc(NC(=O)Cc4cccc(OCCOCCNc5cccc6c5C(=O)N(C5CCC(=O)NC5=O)C6=O)c4)sc3C)ccc21] | 2-(3-(2-(2-((2-(2,6-dioxopiperidin-3- yl)-1,3-dioxoisoindolin-4- yl)amino)ethoxy)ethoxy)phenyl)-N-(5- methyl-4-(1-(3- methylpicolinoyl)indolin-5-yl)thiazol- 2-yl)acetamide |
| 80 | 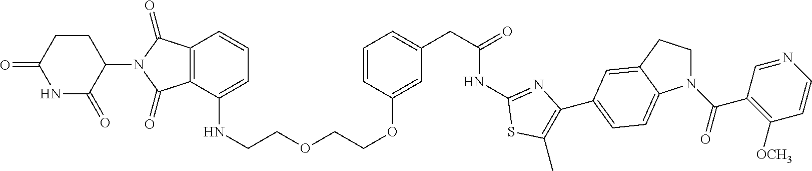 [SMILES: COc1ccncc1C(=O)N1CCc2cc(-c3nc(NC(=O)Cc4cccc(OCCOCCNc5cccc6c5C(=O)N(C5CCC(=O)NC5=O)C6=O)c4)sc3C)ccc21] | 2-(3-(2-(2-((2-(2,6-dioxopiperidin-3- yl)-1,3-dioxoisoindolin-4- yl)amino)ethoxy)ethoxy)phenyl)-N-(4- (1-(4-methoxynicotinoyl)indolin-5-yl)- 5-methylthiazol-2-yl)acetamide |
| 81 | 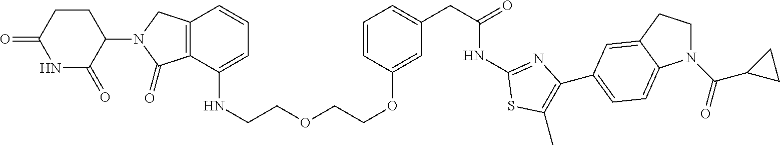 [SMILES: Cc1sc(NC(=O)Cc2cccc(OCCOCCNc3cccc4c3C(=O)N(C3CCC(=O)NC3=O)C4)c2)nc1-c1ccc2c(c1)CCN2C(=O)C1CC1] | N-(4-(1- (cyclopropanecarbonyl)indolin-5-yl)- 5-methylthiazol-2-yl)-2-(3-(2-(2-((2- (2,6-dioxopiperidin-3-yl)-3- oxoisoindolin-4- yl)amino)ethoxy)ethoxy)phenyl) acetamide |
| 82 | 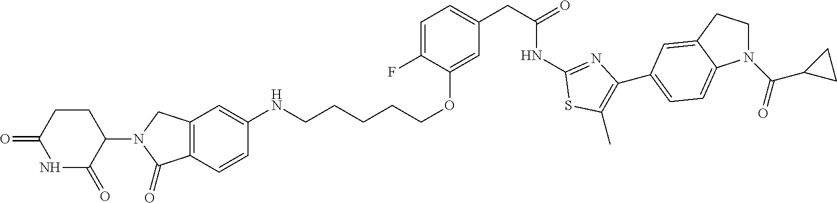 [SMILES: Cc1sc(NC(=O)Cc2ccc(F)c(OCCCCCNc3ccc4c(c3)CN(C3CCC(=O)NC3=O)C4=O)c2)nc1-c1ccc2c(c1)CCN2C(=O)C1CC1] | N-(4-(1- (cyclopropanecarbonyl)indolin-5-yl)- 5-methylthiazol-2-yl)-2-(3-((5-((2- (2,6-dioxopiperidin-3-yl)-1- oxoisoindolin-5-yl)amino)pentyl)oxy)- 4-fluorophenyl)acetamide |
| 83 | 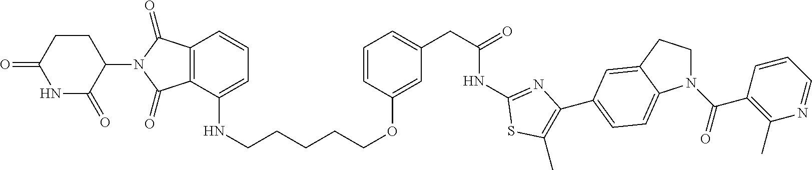 [SMILES: Cc1ncccc1C(=O)N1CCc2cc(-c3nc(NC(=O)Cc4cccc(OCCCCCNc5cccc6c5C(=O)N(C5CCC(=O)NC5=O)C6=O)c4)sc3C)ccc21] | 2-(3-((5-((2-(2,6-dioxopiperidin-3-yl)- 1,3-dioxoisoindolin-4- yl)amino)pentyl)oxy)phenyl)-N-(5- methyl-4-(1-(2- methylnicotinoyl)indolin-5-yl)thiazol- 2-yl)acetamide |
| 84 | 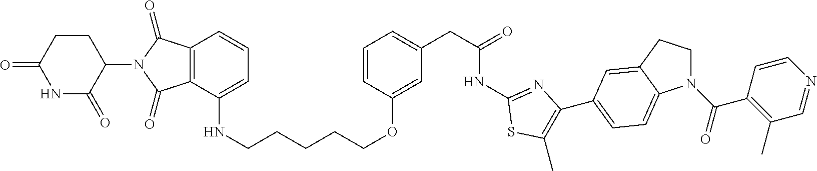 [SMILES: Cc1cnccc1C(=O)N1CCc2cc(-c3nc(NC(=O)Cc4cccc(OCCCCCNc5cccc6c5C(=O)N(C5CCC(=O)NC5=O)C6=O)c4)sc3C)ccc21] | 2-(3-((5-((2-(2,6-dioxopiperidin-3-yl)- 1,3-dioxoisoindolin-4- yl)amino)pentyl)oxy)phenyl)-N-(5- methyl-4-(1-(3- methylisonicotinoyl)indolin-5- yl)thiazol-2-yl)acetamide |
| 85 | 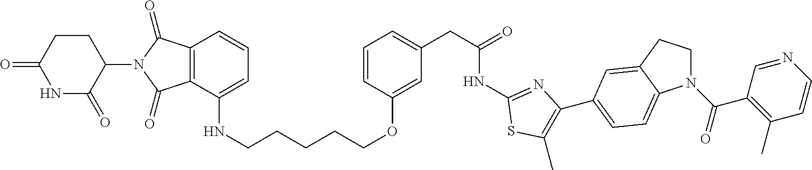 [SMILES: Cc1ccncc1C(=O)N1CCc2cc(-c3nc(NC(=O)Cc4cccc(OCCCCCNc5cccc6c5C(=O)N(C5CCC(=O)NC5=O)C6=O)c4)sc3C)ccc21] | 2-(3-((5-((2-(2,6-dioxopiperidin-3-yl)- 1,3-dioxoisoindolin-4- yl)amino)pentyl)oxy)phenyl)-N-(5- methyl-4-(1-(4- methylnicotinoyl)indolin-5-yl)thiazol- 2-yl)acetamide |
| 86 | 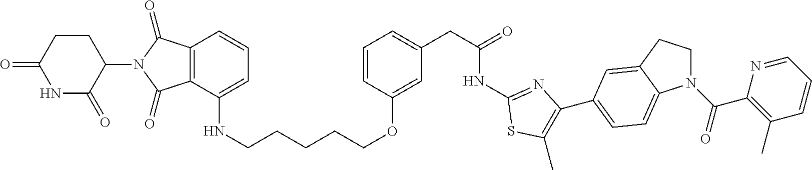 [SMILES: Cc1cccnc1C(=O)N1CCc2cc(-c3nc(NC(=O)Cc4cccc(OCCCCCNc5cccc6c5C(=O)N(C5CCC(=O)NC5=O)C6=O)c4)sc3C)ccc21] | 2-(3-((5-((2-(2,6-dioxopiperidin-3-yl)- 1,3-dioxoisoindolin-4- yl)amino)pentyl)oxy)phenyl)-N-(5- methyl-4-(1-(3- methylpicolinoyl)indolin-5-yl)thiazol- 2-yl)acetamide |
| 87 | 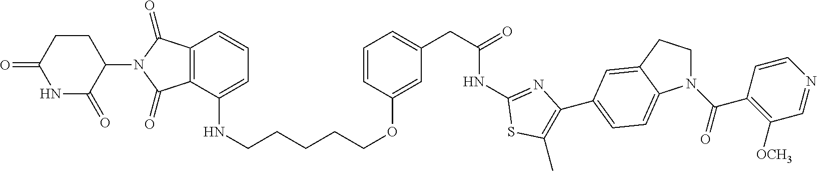 [SMILES: COc1cnccc1C(=O)N1CCc2cc(-c3nc(NC(=O)Cc4cccc(OCCCCCNc5cccc6c5C(=O)N(C5CCC(=O)NC5=O)C6=O)c4)sc3C)ccc21] | 2-(3-((5-((2-(2,6-dioxopiperidin-3-yl)- 1,3-dioxoisoindolin-4- yl)amino)pentyl)oxy)phenyl)-N-(4-(1- (4-methoxynicotinoyl)indolin-5-yl)-5- methylthiazol-2-yl)acetamide |

TABLE 1-A

| Cpd. | | |
|---|---|---|
| No. | Structure | Chemical Name |
| 60 | 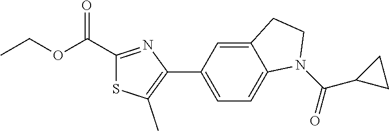 [SMILES: CCOC(=O)c1nc(-c2ccc3c(c2)CCN3C(=O)C2CC2)c(C)s1] | ethyl 4-(1- (cyclopropanecarbonyl) indolin-5-yl)-5- methylthiazole-2- carboxylate |
| 61 | 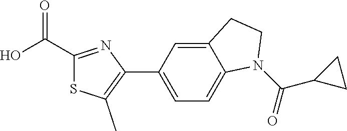 [SMILES: Cc1sc(C(=O)O)nc1-c1ccc2c(c1)CCN2C(=O)C1CC1] | 4-(1-(cyclopropane- carbonyl)indolin-5- yl)-5-methylthiazole- 2-carboxylic acid |
| 62 | 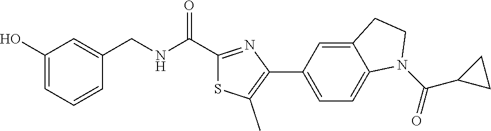 [SMILES: Cc1sc(C(=O)NCc2cccc(O)c2)nc1-c1ccc2c(c1)CCN2C(=O)C1CC1] | 4-(1-(cyclopropane- carbonyl)indolin-5- yl)-N-(3-hydroxybenzyl)-5- methylthiazole-2- carboxamide |
| 63 | 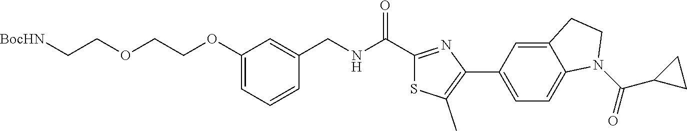 [SMILES: Cc1sc(C(=O)NCc2cccc(OCCOCCNC(=O)OC(C)(C)C)c2)nc1-c1ccc2c(c1)CCN2C(=O)C1CC1] | tert-butyl (2-(2-(3-((4-(1- (cyclopropanecarbonyl) indolin-5-yl)-5- methylthiazole-2- carboxamido)methyl) phenoxy)ethoxy) ethyl)carbamate |
| 64 | 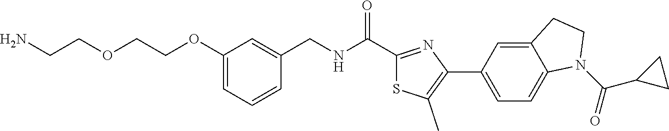 [SMILES: Cc1sc(C(=O)NCc2cccc(OCCOCCN)c2)nc1-c1ccc2c(c1)CCN2C(=O)C1CC1] | N-(3-(2-(2- aminoethoxy)ethoxy) benzyl)-4-(1- (cyclopropanecarbonyl) indolin-5-yl)-5- methylthiazole-2- carboxamide |

In another embodiment, the disclosure provides a pharmaceutical composition comprising a Compound of the Disclosure and a pharmaceutically acceptable carrier or excipient.

In another embodiment, the disclosure provides 2-(3-hydroxyphenyl)-N-(5-methyl-4-(1-(2-methylbenzoyl)indolin-5-yl)thiazol-2-yl)acetamide as a synthetic intermediate to prepare a Compound of the Disclosure.

In another embodiment, the disclosure provides 2-(4-fluoro-3-hydroxyphenyl)-N-(5-methyl-4-(1-((2-nitrophenyl)sulfonyl)indolin-5-yl)thiazol-2-yl)acetamide as a synthetic intermediate to prepare a Compound of the Disclosure.

Compounds of the Disclosure may contain an asymmetric carbon atom. In some embodiments, Compounds of the Disclosure are racemic compounds. In other embodiments, Compounds of the Disclosure are enantiomerically enriched, e.g., the enantiomeric excess or “ee” of the compound is about 5% or more as measured by chiral HPLC. In another embodiment, the ee is about 10%. In another embodiment, the ee is about 20%. In another embodiment, the ee is about 30%. In another embodiment, the ee is about 40%. In another embodiment, the ee is about 50%. In another embodiment, the ee is about 60%. In another embodiment, the ee is about 70%. In another embodiment, the ee is about 80%. In another embodiment, the ee is about 85%. In another embodiment, the ee is about 90%. In another embodiment, the ee is about 91%. In another embodiment, the ee is about 92%. In another embodiment, the ee is about 93%. In another embodiment, the ee is about 94%. In another embodiment, the ee is about 95%. In another embodiment, the ee is about 96%. In another embodiment, the ee is about 97%. In another embodiment, the ee is about 98%. In another embodiment, the ee is about 99%.

In another embodiment, the cereblon binding portion of a Compound of the Disclosure, i.e., B¹, is enantiomerically enriched. In another embodiment, the cereblon binding portion of the molecule is racemic. The present disclosure encompasses all possible stereoisomeric, e.g., diastereomeric, forms of Compounds of the Disclosure. When a Compound of the Disclosure is desired as a single enantiomer, it can be obtained either by resolution of the final product or by stereospecific synthesis from either isomerically pure starting material or use of a chiral auxiliary reagent, for example, see Z. Ma et al., Tetrahedron: Asymmetry, 8(6), pages 883-888 (1997). Resolution of the final product, an intermediate, or a starting material can be achieved by any suitable method known in the art. Additionally, in situations where tautomers of the Compounds of the Disclosure are possible, the present disclosure is intended to include all tautomeric forms of the compounds.

The present disclosure encompasses the preparation and use of salts of Compounds of the Disclosure, including pharmaceutically acceptable salts. As used herein, the “pharmaceutically acceptable salt” refers to non-toxic salt forms of Compounds of the Disclosure. See e.g., Gupta et al., Molecules 23:1719 (2018). Salts of Compounds of the Disclosure can be prepared during the final isolation and purification of the compounds or separately by reacting the compound with an acid having a suitable cation. The pharmaceutically acceptable salts of Compounds of the Disclosure can be acid addition salts formed with pharmaceutically acceptable acids. Examples of acids which can be employed to form pharmaceutically acceptable salts include inorganic acids such as nitric, boric, hydrochloric, hydrobromic, sulfuric, and phosphoric, and organic acids such as oxalic, maleic, succinic, and citric. Nonlimiting examples of salts of compounds of the disclosure include, but are not limited to, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerolphsphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, paratoluenesulfonate, undecanoate, lactate, citrate, tartrate, gluconate, methanesulfonate, ethanedisulfonate, benzene sulfonate, and p-toluenesulfonate salts. In addition, available amino groups present in the compounds of the disclosure can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. In light of the foregoing, any reference Compounds of the Disclosure appearing herein is intended to include the actual compound as well as pharmaceutically acceptable salts, hydrates, or solvates thereof.

The present disclosure also encompasses the preparation and use of solvates of Compounds of the Disclosure. Solvates typically do not significantly alter the physiological activity or toxicity of the compounds, and as such may function as pharmacological equivalents. The term “solvate” as used herein is a combination, physical association and/or solvation of a compound of the present disclosure with a solvent molecule such as, e.g. a disolvate, monosolvate or hemisolvate, where the ratio of solvent molecule to compound of the present disclosure is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. Compounds of the Disclosure can be present as solvated forms with a pharmaceutically acceptable solvent, such as water, methanol, and ethanol, and it is intended that the disclosure includes both solvated and unsolvated forms of Compounds of the Disclosure. One type of solvate is a hydrate. A “hydrate” relates to a particular subgroup of solvates where the solvent molecule is water. Solvates typically can function as pharmacological equivalents. Preparation of solvates is known in the art. See, for example, M. Caira et al, J. Pharmaceut. Si., 93(3):601-611 (2004), which describes the preparation of solvates of fluconazole with ethyl acetate and with water. Similar preparation of solvates, hemisolvates, hydrates, and the like are described by E. C. van Tonder et al., AAPS Pharm. Sci. Tech., 5(1): Article 12 (2004), and A. L. Bingham et al., Chem. Commun. 603-604 (2001). A typical, non-limiting, process of preparing a solvate would involve dissolving a Compound of the Disclosure in a desired solvent (organic, water, or a mixture thereof) at temperatures above 20° C. to about 25° C., then cooling the solution at a rate sufficient to form crystals, and isolating the crystals by known methods, e.g., filtration. Analytical techniques such as infrared spectroscopy can be used to confirm the presence of the solvent in a crystal of the solvate.

### II. Therapeutic Methods of the Disclosure

Compounds of the Disclosure degrade Nrf2 protein and are thus useful in the treatment of a variety of diseases and conditions. In particular, Compounds of the Disclosure are useful in methods of treating a disease or condition wherein degradation of Nrf2 proteins provides a benefit, for example, cancers and proliferative diseases. Compounds of the Disclosure are also useful in methods of treating a neurodegenerative disease, diabetes, a cardiovascular disease, a kidney disease, or a liver disease in a subject.

The therapeutic methods of the disclosure comprise administering a therapeutically effective amount of a Compound of the Disclosure to a subject, e.g., a cancer patient, in need thereof. The present methods also encompass administering a second therapeutic agent to the subject in combination with the Compound of the Disclosure. The second therapeutic agent is selected from drugs known as useful in treating the disease or condition afflicting the individual in need thereof, e.g., a chemotherapeutic agent and/or radiation known as useful in treating a particular cancer.

The present disclosure provides Compounds of the Disclosure as Nrf2 protein degraders for the treatment of diseases and conditions wherein degradation of Nrf2 proteins has a beneficial effect. Compounds of the Disclosure typically have DC₅₀ (the drug concentration that results in 50% Nrf2 protein degradation) values of less than 100 μM, e.g., less than 50 μM, less than 25 μM, and less than 5 μM, less than about 1 μM, less than about 0.5 μM, or less than about 0.1 μM.

In one embodiment, the present disclosure relates to a method of treating an individual suffering from a disease or condition wherein degradation of Nrf2 protein provides a benefit comprising administering a therapeutically effective amount of a Compound of the Disclosure to an individual in need thereof. Since Compounds of the Disclosure are degraders of Nrf2 protein, a number of diseases and conditions mediated by or genetically associated with Nrf2 can be treated by employing these compounds. The present disclosure is thus directed generally to a method for treating a disease or condition responsive to degradation of Nrf2 in an animal, e.g., a human, suffering from, or at risk of suffering from, the disease or condition, the method comprising administering to the animal an effective amount of one or more Compounds of the Disclosure.

The present disclosure is further directed to a method of degrading Nrf2 protein in a subject in need thereof, said method comprising administering to the subject an effective amount of at least one Compound of the Disclosure.

In another aspect, the present disclosure provides a method of treating cancer in a subject comprising administering a therapeutically effective amount of a Compound of the Disclosure. While not being limited to a specific mechanism, in some embodiments, Compounds of the Disclosure treat cancer by degrading Nrf2. Examples of treatable cancers include, but are not limited to, any one or more of the cancers of Table 2.

TABLE 2

| | | | |
|---|---|---|---|
| adrenal cancer | acinic cell carcinoma | acoustic neuroma | acral lentigious |
| | | | melanoma |
| acrospiroma | acute eosinophilic | acute erythroid | acute lymphoblastic |
| | leukemia | leukemia | leukemia |
| acute | acute monocytic | acute promyelocytic | adenocarcinoma |
| megakaryoblastic | leukemia | leukemia | |
| leukemia | | | |
| adenoid cystic | adenoma | adenomatoid | adenosquamous |
| carcinoma | | odontogenic tumor | carcinoma |
| adipose tissue | adrenocortical | adult T-cell | aggressive NK-cell |
| neoplasm | carcinoma | leukemia/lymphoma | leukemia |
| AIDS-related | alveolar | alveolar soft part | ameloblastic |
| lymphoma | rhabdomyosarcoma | sarcoma | fibroma |
| anaplastic large cell | anaplastic thyroid | angioimmunoblastic | angiomyolipoma |
| lymphoma | cancer | T-cell lymphoma | |
| angiosarcoma | astrocytoma | atypical teratoid | B-cell chronic |
| | | rhabdoid tumor | lymphocytic |
| | | | leukemia |
| B-cell | B-cell lymphoma | basal cell carcinoma | biliary tract cancer |
| prolymphocytic | | | |
| leukemia | | | |
| bladder cancer | blastoma | bone cancer | Brenner tumor |
| Brown tumor | Burkitt's lymphoma | breast cancer | brain cancer |
| carcinoma | carcinoma in situ | carcinosarcoma | cartilage tumor |
| cementoma | myeloid sarcoma | chondroma | chordoma |
| choriocarcinoma | choroid plexus | clear-cell sarcoma of | craniopharyngioma |
| | papilloma | the kidney | |
| cutaneous T-cell | cervical cancer | colorectal cancer | Degos disease |
| lymphoma | | | |
| desmoplastic small | diffuse large B-cell | dysembryoplastic | dysgerminoma |
| round cell tumor | lymphoma | neuroepithelial | |
| | | tumor | |
| embryonal | endocrine gland | endodermal sinus | enteropathy- |
| carcinoma | neoplasm | tumor | associated T-cell |
| | | | lymphoma |
| esophageal cancer | fetus in fetu | fibroma | fibrosarcoma |
| follicular | follicular thyroid | ganglioneuroma | gastrointestinal |
| lymphoma | cancer | | cancer |
| germ cell tumor | gestational | giant cell | giant cell tumor of |
| | choriocarcinoma | fibroblastoma | the bone |
| glial tumor | glioblastoma | glioma | gliomatosis cerebri |
| | multiforme | | |
| glucagonoma | gonadoblastoma | granulosa cell tumor | gynandroblastoma |
| gallbladder cancer | gastric cancer | hairy cell leukemia | hemangioblastoma |
| head and neck | hemangiopericytoma | hematological | hepatoblastoma |
| cancer | | cancer | |
| hepatosplenic T-cell | Hodgkin's | non-Hodgkin's | invasive lobular |
| lymphoma | lymphoma | lymphoma | carcinoma |
| intestinal cancer | kidney cancer | laryngeal cancer | lentigo maligna |
| lethal midline | leukemia | leydig cell tumor | liposarcoma |
| carcinoma | | | |
| lung cancer | lymphangioma | lymphangiosarcoma | lymphoepithelioma |
| lymphoma | acute lymphocytic | acute myelogeous | chronic |
| | leukemia | leukemia | lymphocytic |
| | | | leukemia |
| liver cancer | small cell lung | non-small cell lung | MALT lymphoma |
| | cancer | cancer | |
| malignant fibrous | malignant peripheral | malignant triton | mantle cell |
| histiocytoma | nerve sheath tumor | tumor | lymphoma |
| marginal zone B- | mast cell leukemia | mediastinal germ | medullary |
| cell lymphoma | | cell tumor | carcinoma of the |
| | | | breast |
| medullary thyroid | medulloblastoma | melanoma | meningioma |
| cancer | | | |
| merkel cell cancer | mesothelioma | metastatic urothelial | mixed Mullerian |
| | | carcinoma | tumor |
| mucinous tumor | multiple myeloma | muscle tissue | mycosis fungoides |
| | | neoplasm | |
| myxoid | myxoma | myxosarcoma | nasopharyngeal |
| liposarcoma | | | carcinoma |
| neurinoma | neuroblastoma | neurofibroma | neuroma |
| nodular melanoma | ocular cancer | oligoastrocytoma | oligodendroglioma |
| oncocytoma | optic nerve sheath | optic nerve tumor | oral cancer |
| | meningioma | | |
| osteosarcoma | ovarian cancer | Pancoast tumor | papillary thyroid |
| | | | cancer |
| paraganglioma | pinealoblastoma | pineocytoma | pituicytoma |
| pituitary adenoma | pituitary tumor | plasmacytoma | polyembryoma |
| precursor T- | primary central | primary effusion | preimary peritoneal |
| lymphoblastic | nervous system | lymphoma | cancer |
| lymphoma | lymphoma | | |
| prostate cancer | pancreatic cancer | pharyngeal cancer | pseudomyxoma |
| | | | periotonei |
| renal cell carcinoma | renal medullary | retinoblastoma | rhabdomyoma |
| | carcinoma | | |
| rhabdomyosarcoma | Richter's | rectal cancer | sarcoma |
| | transformation | | |
| Schwannomatosis | seminoma | Sertoli cell tumor | sex cord-gonadal |
| | | | stromal tumor |
| signet ring cell | skin cancer | small blue round cell | small cell |
| carcinoma | | tumors | carcinoma |
| soft tissue sarcoma | somatostatinoma | soot wart | spinal tumor |
| splenic marginal | squamous cell | synovial sarcoma | Sezary's disease |
| zone lymphoma | carcinoma | | |
| small intestine | squamous carcinoma | stomach cancer | T-cell lymphoma |
| cancer | | | |
| testicular cancer | thecoma | thyroid cancer | transitional cell |
| | | | carcinoma |
| throat cancer | urachal cancer | urogenital cancer | urothelial |
| | | | carcinoma |
| uveal melanoma | uterine cancer | verrucous carcinoma | visual pathway |
| | | | glioma |
| vulvar cancer | vaginal cancer | Waldenstrom's | Warthin's tumor |
| | | macroglobulinemia | |
| Wilms' tumor | | | |

In another embodiment, the cancer is a solid tumor. In another embodiment, the cancer a hemoatological cancer. Exemplary hematological cancers include, but are not limited to, the cancers listed in Table 3. In another embodiment, the hematological cancer is acute lymphocytic leukemia, chronic lymphocytic leukemia (including B-cell chronic lymphocytic leukemia), or acute myeloid leukemia.

TABLE 3

| | |
|---|---|
| acute lymphocytic leukemia (ALL) | acute eosinophilic leukemia |
| acute myeloid leukemia (AML) | acute erythroid leukemia |
| chronic lymphocytic leukemia (CLL) | acute lymphoblastic leukemia |
| small lymphocytic lymphoma (SLL) | acute megakaryoblastic leukemia |
| multiple myeloma (MM) | acute monocytic leukemia |
| Hodgkins lymphoma (HL) | acute promyelocytic leukemia |
| non-Hodgkin's lymphoma (NHL) | acute myelogeous leukemia |
| mantle cell lymphoma (MCL) | B-cell prolymphocytic leukemia |
| marginal zone B-cell lymphoma | B-cell lymphoma |
| splenic marginal zone lymphoma | MALT lymphoma |
| follicular lymphoma (FL) | precursor T-lymphoblastic |
| | lymphoma |
| Waldenstrom's | T-cell lymphoma |
| macroglobulinemia (WM) | |
| diffuse large B-cell | mast cell leukemia |
| lymphoma (DLBCL) | |
| marginal zone lymphoma (MZL) | adult T cell leukemia/lymphoma |
| hairy cell leukemia (HCL) | aggressive NK-cell leukemia |
| Burkitt's lymphoma (BL) | angioimmunoblastic T-cell |
| | lymphoma |
| Richter's transformation | |

In another embodiment, the cancer is a leukemia, for example a leukemia selected from acute monocytic leukemia, acute myelogenous leukemia, chronic myelogenous leukemia, chronic lymphocytic leukemia and mixed lineage leukemia (MLL). In another embodiment the cancer is NUT-midline carcinoma. In another embodiment the cancer is multiple myeloma. In another embodiment the cancer is a lung cancer such as small cell lung cancer (SCLC). In another embodiment the cancer is a neuroblastoma. In another embodiment the cancer is Burkitt's lymphoma. In another embodiment the cancer is cervical cancer. In another embodiment the cancer is esophageal cancer. In another embodiment the cancer is ovarian cancer. In another embodiment the cancer is colorectal cancer. In another embodiment, the cancer is prostate cancer. In another embodiment, the cancer is breast cancer.

In another embodiment, the cancer is selected from the group consisting of acute monocytic leukemia, acute myelogenous leukemia, chronic myelogenous leukemia, chronic lymphocytic leukemia mixed lineage leukemia, NUT-midline carcinoma, multiple myeloma, small cell lung cancer, non-small cell lung cancer, neuroblastoma, Burkitt's lymphoma, cervical cancer, esophageal cancer, ovarian cancer, colorectal cancer, prostate cancer, breast cancer, bladder cancer, ovary cancer, glioma, sarcoma, esophageal squamous cell carcinoma, and papillary thyroid carcinoma.

In another embodiment, Compounds of the Disclosure are administered to a subject in need thereof to treat lung cancer, breast cancer, ovarian cancer, or prostate cancer. In another embodiment, the cancer is breast cancer. In another embodiment, the cancer is ovarian cancer. In another embodiment, the cancer is prostate cancer. In another embodiment, the cancer is metastatic castration-resistant prostate cancer.

In another aspect, the present disclosure provides a method of treating autoimmune disease, respiratory disease, digestive disease, cardiovascular disease, metabolic disease, or neurodegenerative disease in a subject comprising administering a therapeutically effective amount of a Compound of the Disclosure. While not being limited to a specific mechanism, in some embodiments, Compounds of the Disclosure treat these diseases by degrading Nrf2.

In another aspect, the present disclosure provides a method of treating neurodegenerative disease in a subject comprising administering a therapeutically effective amount of a Compound of the Disclosure.

In another aspect, the present disclosure provides a method of treating diabetes in a subject comprising administering a therapeutically effective amount of a Compound of the Disclosure.

In another aspect, the present disclosure provides a method of treating cardiovascular disease in a subject comprising administering a therapeutically effective amount of a Compound of the Disclosure.

In another aspect, the present disclosure provides a method of treating kidney disease in a subject comprising administering a therapeutically effective amount of a Compound of the Disclosure.

In another aspect, the present disclosure provides a method of treating liver disease in a subject comprising administering a therapeutically effective amount of a Compound of the Disclosure.

The methods of the present disclosure can be accomplished by administering a Compound of the Disclosure as the neat compound or as a pharmaceutical composition. Administration of a pharmaceutical composition, or neat Compound of the Disclosure, can be performed during or after the onset of the disease or condition of interest. Typically, the pharmaceutical compositions are sterile, and contain no toxic, carcinogenic, or mutagenic compounds that would cause an adverse reaction when administered.

In one embodiment, a Compound of the Disclosure is administered as a single agent to treat a disease or condition wherein degradation of Nrf2 protein provides a benefit. In another embodiment, a Compound of the Disclosure is administered in conjunction with a second therapeutic agent useful in the treatment of a disease or condition wherein degradation of Nrf2 protein provides a benefit. The second therapeutic agent is different from the Compound of the Disclosure. A Compound of the Disclosure and the second therapeutic agent can be administered simultaneously or sequentially to achieve the desired effect. In addition, the Compound of the Disclosure and second therapeutic agent can be administered as a single pharmaceutical composition or two separate pharmaceutical compositions.

The second therapeutic agent is administered in an amount to provide its desired therapeutic effect. The effective dosage range for each second therapeutic agent is known in the art, and the second therapeutic agent is administered to an individual in need thereof within such established ranges.

A Compound of the Disclosure and the second therapeutic agent can be administered together as a single-unit dose or separately as multi-unit doses, wherein the Compound of the Disclosure is administered before the second therapeutic agent or vice versa. One or more doses of the Compound of the Disclosure and/or one or more doses of the second therapeutic agent can be administered. The Compound of the Disclosure therefore can be used in conjunction with one or more second therapeutic agents, for example, but not limited to, anticancer agents.

In methods of the present disclosure, a therapeutically effective amount of a Compound of the Disclosure, typically formulated in accordance with pharmaceutical practice, is administered to a subject, e.g., a human cancer patient, in need thereof. Whether such a treatment is indicated depends on the individual case and is subject to medical assessment (diagnosis) that takes into consideration signs, symptoms, and/or malfunctions that are present, the risks of developing particular signs, symptoms and/or malfunctions, and other factors.

A Compound of the Disclosure can be administered by any suitable route, for example by oral, buccal, inhalation, sublingual, rectal, vaginal, intracisternal or intrathecal through lumbar puncture, transurethral, nasal, percutaneous, i.e., transdermal, or parenteral (including intravenous, intramuscular, subcutaneous, intracoronary, intradermal, intramammary, intraperitoneal, intraarticular, intrathecal, retrobulbar, intrapulmonary injection and/or surgical implantation at a particular site) administration. Parenteral administration can be accomplished using a needle and syringe or using a high pressure technique.

Pharmaceutical compositions include those wherein a Compound of the Disclosure is administered in an effective amount to achieve its intended purpose. The exact formulation, route of administration, and dosage is determined by an individual physician in view of the diagnosed condition or disease. Dosage amount and interval can be adjusted individually to provide levels of a Compound of the Disclosure that is sufficient to maintain therapeutic effects.

Toxicity and therapeutic efficacy of the Compounds of the Disclosure can be determined by standard pharmaceutical procedures in cell cultures or experimental animals, e.g., for determining the maximum tolerated dose (MTD) of a compound, which is defined as the highest tolerated dose of a chemical that can be administered to animals without causing severe toxicity or mortality. The dose ratio between the maximum tolerated dose and therapeutic effects (e.g. inhibiting of tumor growth) is the therapeutic index. The dosage can vary within this range depending upon the dosage form employed, and the route of administration utilized. Determination of a therapeutically effective amount is well within the capability of those skilled in the art, especially in light of the detailed disclosure provided herein.

A therapeutically effective amount of a Compound of the Disclosure required for use in therapy varies with the nature of the condition being treated, the length of time that activity is desired, and the age and the condition of the patient, and ultimately is determined by the attendant physician. Dosage amounts and intervals can be adjusted individually to provide plasma levels of the Nrf2 protein degrader that are sufficient to maintain the desired therapeutic effects. The desired dose conveniently can be administered in a single dose, or as multiple doses administered at appropriate intervals, for example as one, two, three, four or more subdoses per day. Multiple doses often are desired, or required. For example, a Compound of the Disclosure can be administered at a frequency of four doses delivered as one dose per day at four-day intervals (q4d×4); four doses delivered as one dose per day at three-day intervals (q3d×4); one dose delivered per day at five-day intervals (qd×5); one dose per week for three weeks (qwk3); five daily doses, with two days rest, and another five daily doses (5/2/5); or, any dose regimen determined to be appropriate for the circumstance.

A Compound of the Disclosure used in a method of the present disclosure can be administered in an amount of about 0.005 to about 500 milligrams per dose, about 0.05 to about 250 milligrams per dose, or about 0.5 to about 100 milligrams per dose. For example, a Compound of the Disclosure can be administered, per dose, in an amount of about 0.005, 0.05, 0.5, 5, 10, 20, 30, 40, 50, 100, 150, 200, 250, 300, 350, 400, 450, or 500 milligrams, including all doses between 0.005 and 500 milligrams.

The dosage of a composition containing a Compound of the Disclosure, or a composition containing the same, can be from about 1 ng/kg to about 200 mg/kg, about 1 μg/kg to about 100 mg/kg, or about 1 mg/kg to about 50 mg/kg. The dosage of a composition can be at any dosage including, but not limited to, about 1 μg/kg. The dosage of a composition may be at any dosage including, but not limited to, about 1 μg/kg, about 10 g/kg, about 25 μg/kg, about 50 μg/kg, about 75 μg/kg, about 100 μg/kg, about 125 μg/kg, about 150 μg/kg, about 175 μg/kg, about 200 μg/kg, about 225 μg/kg, about 250 μg/kg, about 275 μg/kg, about 300 μg/kg, about 325 g/kg, about 350 μg/kg, about 375 μg/kg, about 400 μg/kg, about 425 μg/kg, about 450 μg/kg, about 475 μg/kg, about 500 μg/kg, about 525 μg/kg, about 550 μg/kg, about 575 μg/kg, about 600 μg/kg, about 625 μg/kg, about 650 μg/kg, about 675 μg/kg, about 700 μg/kg, about 725 μg/kg, about 750 μg/kg, about 775 μg/kg, about 800 μg/kg, about 825 μg/kg, about 850 μg/kg, about 875 μg/kg, about 900 μg/kg, about 925 μg/kg, about 950 μg/kg, about 975 μg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 mg/kg, about 100 mg/kg, about 125 mg/kg, about 150 mg/kg, about 175 mg/kg, about 200 mg/kg, or more. The above dosages are exemplary of the average case, but there can be individual instances in which higher or lower dosages are merited, and such are within the scope of this disclosure. In practice, the physician determines the actual dosing regimen that is most suitable for an individual patient, which can vary with the age, weight, and response of the particular patient.

As stated above, a Compound of the Disclosure can be administered in combination with a second therapeutically active agent. In some embodiments, the second therapeutic agent is an epigenetic drug. As used herein, the term “epigenetic drug” refers to a therapeutic agent that targets an epigenetic regulator. Examples of epigenetic regulators include the histone lysine methyltransferases, histone arginine methyl transferases, histone demethylases, histone deacetylases, histone acetylases, and DNA methyltransferases. Histone deacetylase inhibitors include, but are not limited to, vorinostat.

In another embodiment, chemotherapeutic agents or other anti-proliferative agents can be combined with Compound of the Disclosure to treat proliferative diseases and cancer. Examples of therapies and anticancer agents that can be used in combination with Compounds of the Disclosure include surgery, radiotherapy (e.g., gamma-radiation, neutron beam radiotherapy, electron beam radiotherapy, proton therapy, brachytherapy, and systemic radioactive isotopes), endocrine therapy, a biologic response modifier (e.g., an interferon, an interleukin, tumor necrosis factor (TNF), hyperthermia and cryotherapy, an agent to attenuate any adverse effect (e.g., an antiemetic), and any other approved chemotherapeutic drug.

Examples of antiproliferative compounds include, but are not limited to, an aromatase inhibitor; an anti-estrogen; an anti-androgen; a gonadorelin agonist; a topoisomerase I inhibitor; a topoisomerase II inhibitor; a microtubule active agent; an alkylating agent; a retinoid, a carontenoid, or a tocopherol; a cyclooxygenase inhibitor; an MMP inhibitor; an mTOR inhibitor; an antimetabolite; a platin compound; a methionine aminopeptidase inhibitor; a bisphosphonate; an antiproliferative antibody; a heparanase inhibitor; an inhibitor of Ras oncogenic isoforms; a telomerase inhibitor; a proteasome inhibitor; a compound used in the treatment of hematologic malignancies; a Flt-3 inhibitor; an Hsp90 inhibitor; a kinesin spindle protein inhibitor; a MEK inhibitor; an antitumor antibiotic; a nitrosourea; a compound targeting/decreasing protein or lipid kinase activity, a compound targeting/decreasing protein or lipid phosphatase activity, or any further anti-angiogenic compound.

Nonlimiting exemplary aromatase inhibitors include, but are not limited to, steroids, such as atamestane, exemestane, and formestane, and non-steroids, such as aminogiutethimide, roglethimide, pyridoglutethimide, trilostane, testolactone, ketokonazole, vorozole, fadrozole, anastrozole, and letrozole.

Nonlimiting anti-estrogens include, but are not limited to, tamoxifen, fulvestrant, raloxifene, and raloxifene hydrochloride. Anti-androgens include, but are not limited to, bicalutamide. Gonadorelin agonists include, but are not limited to, abarelix, goserelin, and goserelin acetate.

Exemplary topoisomerase I inhibitors include, but are not limited to, topotecan, gimatecan, irinotecan, camptothecin and its analogues, 9-nitrocamptothecin, and the macromolecular camptothecin conjugate PNU-166148. Topoisomerase II inhibitors include, but are not limited to, anthracyclines, such as doxorubicin, daunorubicin, epirubicin, idarubicin, and nemorubicin; anthraquinones, such as mitoxantrone and losoxantrone; and podophillotoxines, such as etoposide and teniposide.

Microtubule active agents include microtubule stabilizing, microtubule destabilizing compounds, and microtubulin polymerization inhibitors including, but not limited to, taxanes, such as paclitaxel and docetaxel; vinca alkaloids, such as vinblastine, vinblastine sulfate, vincristine, and vincristine sulfate, and vinorelbine; discodermolides; cochicine and epothilones and derivatives thereof.

Exemplary nonlimiting alkylating agents include cyclophosphamide, ifosfamide, melphalan, and nitrosoureas, such as carmustine and lomustine.

Exemplary nonlimiting cyclooxygenase inhibitors include Cox-2 inhibitors, 5-alkyl substituted 2-arylaminophenylacetic acid and derivatives, such as celecoxib, rofecoxib, etoricoxib, valdecoxib, or a 5-alkyl-2-arylaminophenylacetic acid, such as lumiracoxib.

Exemplary nonlimiting matrix metalloproteinase inhibitors (“MMP inhibitors”) include collagen peptidomimetic and nonpeptidomimetic inhibitors, tetracycline derivatives, batimastat, marimastat, prinomastat, metastat, BMS-279251, BAY 12-9566, TAA211, MMI270B, and AAJ996.

Exemplary nonlimiting mTOR inhibitors include compounds that inhibit the mammalian target of rapamycin (mTOR) and possess antiproliferative activity such as sirolimus, everolimus, CCI-779, and ABT578.

Exemplary nonlimiting antimetabolites include 5-fluorouracil (5-FU), capecitabine, gemcitabine, DNA demethylating compounds, such as 5-azacytidine and decitabine, methotrexate and edatrexate, and folic acid antagonists, such as pemetrexed.

Exemplary nonlimiting platin compounds include carboplatin, cis-platin, cisplatinum, and oxaliplatin.

Exemplary nonlimiting methionine aminopeptidase inhibitors include bengamide or a derivative thereof and PPI-2458.

Exemplary nonlimiting bisphosphonates include etridonic acid, clodronic acid, tiludronic acid, pamidronic acid, alendronic acid, ibandronic acid, risedronic acid, and zoledronic acid.

Exemplary nonlimiting antiproliferative antibodies include trastuzumab, trastuzumab-DMI, cetuximab, bevacizumab, rituximab, PR064553, and 2C4. The term “antibody” is meant to include intact monoclonal antibodies, polyclonal antibodies, multispecific antibodies formed from at least two intact antibodies, and antibody fragments, so long as they exhibit the desired biological activity.

Exemplary nonlimiting heparanase inhibitors include compounds that target, decrease, or inhibit heparin sulfate degradation, such as PI-88 and OGT2115.

The term “an inhibitor of Ras oncogenic isoforms,” such as H-Ras, K-Ras, or N-Ras, as used herein refers to a compound which targets, decreases, or inhibits the oncogenic activity of Ras, for example, a farnesyl transferase inhibitor, such as L-744832, DK8G557, tipifarnib, and lonafarnib.

Exemplary nonlimiting telomerase inhibitors include compounds that target, decrease, or inhibit the activity of telomerase, such as compounds that inhibit the telomerase receptor, such as telomestatin.

Exemplary nonlimiting proteasome inhibitors include compounds that target, decrease, or inhibit the activity of the proteasome including, but not limited to, bortezomid.

The phrase “compounds used in the treatment of hematologic malignancies” as used herein includes FMS-like tyrosine kinase inhibitors, which are compounds targeting, decreasing or inhibiting the activity of FMS-like tyrosine kinase receptors (Flt-3R); interferon, I-β-D-arabinofuransylcytosine (ara-c), and bisulfan; and ALK inhibitors, which are compounds which target, decrease, or inhibit anaplastic lymphoma kinase.

Exemplary nonlimiting Flt-3 inhibitors include PKC412, midostaurin, a staurosporine derivative, SUI1248, and MLN518.

Exemplary nonlimiting HSP90 inhibitors include compounds targeting, decreasing, or inhibiting the intrinsic ATPase activity of HSP90; or degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins, or antibodies that inhibit the ATPase activity of HSP90, such as 17-allylamino,17-demethoxygeldanamycin (17AAG), a geldanamycin derivative; other geldanamycin related compounds; radicicol and HDAC inhibitors.

The phrase “a compound targeting/decreasing a protein or lipid kinase activity; or a protein or lipid phosphatase activity; or any further anti-angiogenic compound” as used herein includes a protein tyrosine kinase and/or serine and/or threonine kinase inhibitor or lipid kinase inhibitor, such as a) a compound targeting, decreasing, or inhibiting the activity of the platelet-derived growth factor-receptors (PDGFR), such as a compound that targets, decreases, or inhibits the activity of PDGFR, such as an N-phenyl-2-pyrimidine-amine derivatives, such as imatinib, SUlOl, SU6668, and GFB-111; b) a compound targeting, decreasing, or inhibiting the activity of the fibroblast growth factor-receptors (FGFR); c) a compound targeting, decreasing, or inhibiting the activity of the insulin-like growth factor receptor I (IGF-IR), such as a compound that targets, decreases, or inhibits the activity of IGF-TR; d) a compound targeting, decreasing, or inhibiting the activity of the Trk receptor tyrosine kinase family, or ephrin B4 inhibitors; e) a compound targeting, decreasing, or inhibiting the activity of the Axl receptor tyrosine kinase family; f) a compound targeting, decreasing, or inhibiting the activity of the Ret receptor tyrosine kinase; g) a compound targeting, decreasing, or inhibiting the activity of the Kit/SCFR receptor tyrosine kinase, such as imatinib; h) a compound targeting, decreasing, or inhibiting the activity of the c-Kit receptor tyrosine kinases, such as imatinib; i) a compound targeting, decreasing, or inhibiting the activity of members of the c-Abl family, their gene-fusion products (e.g. Bcr-Abl kinase) and mutants, such as an N-phenyl-2-pyrimidine-amine derivative, such as imatinib or nilotinib; PD180970; AG957; NSC 680410; PD173955; or dasatinib; j) a compound targeting, decreasing, or inhibiting the activity of members of the protein kinase C (PKC) and Raf family of serine/threonine kinases, members of the MEK, SRC, JAK, FAK, PDK1, PKB/Akt, and Ras/MAPK family members, and/or members of the cyclin-dependent kinase family (CDK), such as a staurosporine derivative disclosed in U.S. Pat. No. 5,093,330, such as midostaurin; examples of further compounds include UCN-01, safingol, BAY 43-9006, bryostatin 1, perifosine; ilmofosine; RO 318220 and RO 320432; GO 6976; Isis 3521; LY333531/LY379196; a isochinoline compound; a farnesyl transferase inhibitor; PD184352 or QAN697, or AT7519; k) a compound targeting, decreasing or inhibiting the activity of a protein-tyrosine kinase, such as imatinib mesylate or a tyrphostin, such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) a compound targeting, decreasing, or inhibiting the activity of the epidermal growth factor family of receptor tyrosine kinases (EGFR, ErbB2, ErbB3, ErbB4 as homo- or heterodimers) and their mutants, such as CP 358774, ZD 1839, ZM 105180; trastuzumab, cetuximab, gefitinib, erlotinib, OSI-774, C1-1033, EKB-569, GW-2016, antibodies E1.1, E2.4, E2.5, E6.2, E6.4, E2.11, E6.3 and E7.6.3, and 7H-pyrrolo-[2,3-d]pyrimidine derivatives; and m) a compound targeting, decreasing, or inhibiting the activity of the c-Met receptor.

Exemplary compounds that target, decrease, or inhibit the activity of a protein or lipid phosphatase include inhibitors of phosphatase 1, phosphatase 2A, or CDC25, such as okadaic acid or a derivative thereof.

Further anti-angiogenic compounds include compounds having another mechanism for their activity unrelated to protein or lipid kinase inhibition, e.g., thalidomide and TNP-470.

Additional, nonlimiting, exemplary chemotherapeutic compounds, one or more of which may be used in combination with a Compound of the Disclosure, include: daunorubicin, adriamycin, Ara-C, VP-16, teniposide, mitoxantrone, idarubicin, carboplatinum, PKC412, 6-mercaptopurine (6-MP), fludarabine phosphate, octreotide, SOM230, FTY720, 6-thioguanine, cladribine, 6-mercaptopurine, pentostatin, hydroxyurea, 2-hydroxy-1H-isoindole-1,3-dione derivatives, 1-(4-chloroanilino)-4-(4-pyridylmethyl)phthalazine or a pharmaceutically acceptable salt thereof, 1-(4-chloroanilino)-4-(4-pyridylmethyl)phthalazine succinate, angiostatin, endostatin, anthranilic acid amides, ZD4190, ZD6474, SU5416, SU6668, bevacizumab, rhuMAb, rhuFab, macugon; FLT-4 inhibitors, FLT-3 inhibitors, VEGFR-2 IgGI antibody, RPI 4610, bevacizumab, porfimer sodium, anecortave, triamcinolone, hydrocortisone, 11-a-epihydrocotisol, cortex olone, 17a-hydroxyprogesterone, corticosterone, desoxycorticosterone, testosterone, estrone, dexamethasone, fluocinolone, a plant alkaloid, a hormonal compound and/or antagonist, a biological response modifier, such as a lymphokine or interferon, an antisense oligonucleotide or oligonucleotide derivative, shRNA, and siRNA.

Other examples of second therapeutic agents, one or more of which a Compound of the Disclosure also can be combined, include, but are not limited to: a treatment for Alzheimer's Disease, such as donepezil and rivastigmine; a treatment for Parkinson's Disease, such as L-DOPA/carbidopa, entacapone, ropinrole, pramipexole, bromocriptine, pergolide, trihexephendyl, and amantadine; an agent for treating multiple sclerosis (MS) such as beta interferon (e.g., AVONEX® and REBIF®), glatiramer acetate, and mitoxantrone; a treatment for asthma, such as albuterol and montelukast; an agent for treating schizophrenia, such as zyprexa, risperdal, seroquel, and haloperidol; an anti-inflammatory agent, such as a corticosteroid, a TNF blocker, IL-1 RA, azathioprine, cyclophosphamide, and sulfasalazine; an immunomodulatory agent, including immunosuppressive agents, such as cyclosporin, tacrolimus, rapamycin, mycophenolate mofetil, an interferon, a corticosteroid, cyclophosphamide, azathioprine, and sulfasalazine; a neurotrophic factor, such as an acetylcholinesterase inhibitor, an MAO inhibitor, an interferon, an anti-convulsant, an ion channel blocker, riluzole, or an anti-Parkinson's agent; an agent for treating cardiovascular disease, such as a beta-blocker, an ACE inhibitor, a diuretic, a nitrate, a calcium channel blocker, or a statin; an agent for treating liver disease, such as a corticosteroid, cholestyramine, an interferon, and an anti-viral agent; an agent for treating blood disorders, such as a corticosteroid, an anti-leukemic agent, or a growth factor; or an agent for treating immunodeficiency disorders, such as gamma globulin.

In another embodiment, the second therapeutically active agent is an immune checkpoint inhibitor. Examples of immune checkpoint inhibitors include PD-1 inhibitors, PD-L1 inhibitors, CTLA-4 inhibitors, LAG3 inhibitors, TIM3 inhibitors, cd47 inhibitors, and B7-H1 inhibitors. Thus, in one embodiment, a Compound of the Disclosure is administered in combination with an immune checkpoint inhibitor is selected from the group consisting of a PD-1 inhibitor, a PD-L1 inhibitor, a CTLA-4 inhibitor, a LAG3 inhibitor, a TIM3 inhibitor, and a cd47 inhibitor.

In another embodiment, the immune checkpoint inhibitor is a programmed cell death (PD-1) inhibitor. PD-1 is a T-cell coinhibitory receptor that plays a pivotal role in the ability of tumor cells to evade the host's immune system. Blockage of interactions between PD-1 and PD-L1, a ligand of PD-1, enhances immune function and mediates antitumor activity. Examples of PD-1 inhibitors include antibodies that specifically bind to PD-1. Particular anti-PD-1 antibodies include, but are not limited to nivolumab, pembrolizumab, STI-A1014, and pidilzumab. For a general discussion of the availability, methods of production, mechanism of action, and clinical studies of anti-PD-1 antibodies, see U.S. 2013/0309250, U.S. Pat. Nos. 6,808,710, 7,595,048, 8,008,449, 8,728,474, 8,779,105, 8,952,136, 8,900,587, 9,073,994, 9,084,776, and Naido et al., British Journal of Cancer 111:2214-19 (2014).

In another embodiment, the immune checkpoint inhibitor is a PD-L1 (also known as B7-H1 or CD274) inhibitor. Examples of PD-L1 inhibitors include antibodies that specifically bind to PD-L1. Particular anti-PD-L1 antibodies include, but are not limited to, avelumab, atezolizumab, durvalumab, and BMS-936559. For a general discussion of the availability, methods of production, mechanism of action, and clinical studies, see U.S. Pat. No. 8,217,149, U.S. 2014/0341917, U.S. 2013/0071403, WO 2015036499, and Naido et al., British Journal of Cancer 111:2214-19 (2014).

In another embodiment, the immune checkpoint inhibitor is a CTLA-4 inhibitor. CTLA-4, also known as cytotoxic T-lymphocyte antigen 4, is a protein receptor that downregulates the immune system. CTLA-4 is characterized as a “brake” that binds costimulatory molecules on antigen-presenting cells, which prevents interaction with CD28 on T cells and also generates an overtly inhibitory signal that constrains T cell activation. Examples of CTLA-4 inhibitors include antibodies that specifically bind to CTLA-4. Particular anti-CTLA-4 antibodies include, but are not limited to, ipilimumab and tremelimumab. For a general discussion of the availability, methods of production, mechanism of action, and clinical studies, see U.S. Pat. Nos. 6,984,720, 6,207,156, and Naido et al., British Journal of Cancer 111:2214-19 (2014).

In another embodiment, the immune checkpoint inhibitor is a LAG3 inhibitor. LAG3, Lymphocyte Activation Gene 3, is a negative co-simulatory receptor that modulates T cell homeostatis, proliferation, and activation. In addition, LAG3 has been reported to participate in regulatory T cells (Tregs) suppressive function. A large proportion of LAG3 molecules are retained in the cell close to the microtubule-organizing center, and only induced following antigen specific T cell activation. U.S. 2014/0286935. Examples of LAG3 inhibitors include antibodies that specifically bind to LAG3. Particular anti-LAG3 antibodies include, but are not limited to, GSK2831781. For a general discussion of the availability, methods of production, mechanism of action, and studies, see, U.S. 2011/0150892, U.S. 2014/0093511, U.S. 20150259420, and Huang et al., Immunity 21:503-13 (2004).

In another embodiment, the immune checkpoint inhibitor is a TIM3 inhibitor. TIM3, T-cell immunoglobulin and mucin domain 3, is an immune checkpoint receptor that functions to limit the duration and magnitude of T_{H}l and T_{C}l T-cell responses. The TIM3 pathway is considered a target for anticancer immunotherapy due to its expression on dysfunctional CD8⁺ T cells and Tregs, which are two reported immune cell populations that constitute immunosuppression in tumor tissue. Anderson, Cancer Immunology Research 2:393-98 (2014). Examples of TIM3 inhibitors include antibodies that specifically bind to TIM3. For a general discussion of the availability, methods of production, mechanism of action, and studies of TIM3 inhibitors, see U.S. 20150225457, U.S. 20130022623, U.S. Pat. No. 8,522,156, Ngiow et al., Cancer Res 71: 6567-71 (2011), Ngiow, et al., Cancer Res 71:3540-51 (2011), and Anderson, Cancer Immununology Res 2:393-98 (2014).

In another embodiment, the immune checkpoint inhibitor is a cd47 inhibitor. See Unanue, E. R., PNAS 110:10886-87 (2013).

The term “antibody” is meant to include intact monoclonal antibodies, polyclonal antibodies, multispecific antibodies formed from at least two intact antibodies, and antibody fragments, so long as they exhibit the desired biological activity. In another embodiment, “antibody” is meant to include soluble receptors that do not possess the Fc portion of the antibody. In one embodiment, the antibodies are humanized monoclonal antibodies and fragments thereof made by means of recombinant genetic engineering.

Another class of immune checkpoint inhibitors include polypeptides that bind to and block PD-1 receptors on T-cells without triggering inhibitor signal transduction. Such peptides include B7-DC polypeptides, B7-H1 polypeptides, 137-1 polypeptides and B7-2 polypeptides, and soluble fragments thereof, as disclosed in U.S. Pat. No. 8,114,845.

Another class of immune checkpoint inhibitors include compounds with peptide moieties that inhibit PD-1 signaling. Examples of such compounds are disclosed in U.S. Pat. No. 8,907,053.

Another class of immune checkpoint inhibitors include inhibitors of certain metabolic enzymes, such as indoleamine 2,3 dioxygenase (IDO), which is expressed by infiltrating myeloid cells and tumor cells. The IDO enzyme inhibits immune responses by depleting amino acids that are necessary for anabolic functions in T cells or through the synthesis of particular natural ligands for cytosolic receptors that are able to alter lymphocyte functions. Pardoll, Nature Reviews. Cancer 12:252-64 (2012); Löb, Cancer Immunol Imnunother 58:153-57 (2009). Particular IDO blocking agents include, but are not limited to, levo-1-methyl typtophan (L-1MT) and 1-methyl-tryptophan (1MT). Qian et al., Cancer Res 69:5498-504 (2009); and Löb et al., Cancer Immunol Immunother 58:153-7 (2009).

In one embodiment, the immune checkpoint inhibitor is nivolumab, pembrolizumab, pidilizumab, STI-A1110, avelumab, atezolizumab, durvalumab, STI-A1014, ipilimumab, tremelimumab, GSK2831781, BMS-936559 or MED14736

Other examples of second therapeutic agents, one or more of which a Compound of the Disclosure also can be combined, include glutamine antagonists. Suitable glutamine antagonists are disclosed, for example, in WO 2017/023774 and WO 2019/071110. Particular glutamine antagonists include, but are not limited to, isopropyl (S)-2-((S)-2-acetamido-3-(1H-indol-3-yl)propanamido)-6-diazo-5-oxohexanoate, or a pharmaceutically acceptable salt thereof, isopropyl (S)-2-((S)-6-acetamido-2-((3S,5S,7S)-adamantane-1-carboxamido)hexanamido)-6-diazo-5-oxohexanoate, or a pharmaceutically acceptable salt thereof, (S)-2-((S)-2-acetamido-3-(1H-indol-3-yl)propanamido)-6-diazo-5-oxohexanoic acid, or a pharmaceutically acceptable salt thereof, or 6-diazo-5-oxo-L-norleucine, or a pharmaceutically acceptable salt thereof.

In one embodiment, the second therapeutic agent comprises one of the anti-cancer drugs or anti-cancer drug combinations listed in Table 6.

TABLE 6

| | | | |
|---|---|---|---|
| Abemaciclib | Abiraterone | Abraxane | ABVD |
| | Acetate | (Paclitaxel | |
| | | Albumin-stabilized | |
| | | Nanoparticle Formulation) | |
| ABVE | ABVE-PC | AC | Acalabrutinib |
| AC-T | Actemra | Adcetris | ADE |
| | (Tocilizumab) | (Brentuximab Vedotin) | |
| Ado-Trastuzumab | Adriamycin | Afatinib Dimaleate | Afinitor |
| Emtansine | (Doxorubicin | | (Everolimus) |
| | Hydrochloride) | | |
| Akynzeo | Aldara | Aldesleukin | Alecensa |
| (Netupitant and | (Imiquimod) | | (Alectinib) |
| Palonosetron | | | |
| Hydrochloride) | | | |
| Alectinib | Alemtuzumab | Alimta | Aliqopa |
| | | (Pemetrexed Disodium) | (Copanlisib |
| | | | Hydrochloride) |
| Alkeran for | Alkeran Tablets | Aloxi | Alunbrig |
| Injection | (Melphalan) | (Palonosetron | (Brigatinib) |
| (Melphalan | | Hydrochloride) | |
| Hydrochloride) | | | |
| Ameluz | Amifostine | Aminolevulinic Acid | Anastrozole |
| (Aminolevulinic | | | |
| Acid) | | | |
| Apalutamide | Aprepitant | Aranesp | Aredia |
| | | (Darbepoetin Alfa) | (Pamidronate |
| | | | Disodium) |
| Arimidex | Aromasin | Arranon | Arsenic Trioxide |
| (Anastrozole) | (Exemestane) | (Nelarabine) | |
| Arzerra | Asparaginase | Atezolizumab | Avastin |
| (Ofatumumab) | Erwinia | | (Bevacizumab) |
| | chrysanthemi | | |
| Avelumab | Axicabtagene | Axitinib | Azacitidine |
| | Ciloleucel | | |
| Azedra | Bavencio | BEACOPP | Beleodaq |
| (Iobenguane I 131) | (Avelumab) | | (Belinostat) |
| Belinostat | Bendamustine | Bendeka | BEP |
| | Hydrochloride | (Bendamustine | |
| | | Hydrochloride) | |
| Besponsa | Bevacizumab | Bexarotene | Bicalutamide |
| (Inotuzumab | | | |
| Ozogamicin) | | | |
| BICNU | Binimetinib | Bleomycin | Blinatumomab |
| (Carmustine) | | | |
| Blincyto | Bortezomib | Bosulif | Bosutinib |
| (Blinatumomab) | | (Bosutinib) | |
| Braftovi | Brentuximab | Brigatinib | BuMel |
| (Encorafenib) | Vedotin | | |
| Busulfan | Busulfex | Cabazitaxel | Cabometyx |
| | (Busulfan) | | (Cabozantinib-S- |
| | | | Malate) |
| Cabozantinib-S- | CAF | Calquence | Campath |
| Malate | | (Acalabrutinib) | (Alemtuzumab) |
| Camptosar | Capecitabine | CAPOX | Carac |
| (Irinotecan | | | (Fluorouracil-- |
| Hydrochloride) | | | Topical) |
| Carboplatin | CARBOPLATIN- | Carfilzomib | Carmustine |
| | TAXOL | | |
| Carmustine | Casodex | CEM | Cemiplimab-rwlc |
| Implant | (Bicalutamide) | | |
| Ceritinib | Cerubidine | Cervarix | Cetuximab |
| | (Daunorubicin | (Recombinant HPV | |
| | Hydrochloride) | Bivalent Vaccine) | |
| CEV | Chlorambucil | CHLORAMBUCIL- | CHOP |
| | | PREDNISONE | |
| Cisplatin | Cladribine | Clofarabine | Clolar |
| | | | (Clofarabine) |
| CMF | Cobimetinib | Cometriq | Copanlisib |
| | | (Cabozantinib- | Hydrochloride |
| | | S-Malate) | |
| COPDAC | Copiktra | COPP | COPP-ABV |
| | (Duvelisib) | | |
| Cosmegen | Cotellic | Crizotinib | CVP |
| (Dactinomycin) | (Cobimetinib) | | |
| Cyclophosphamide | Cyramza | Cytarabine | Cytarabine |
| | (Ramucirumab) | | Liposome |
| Cytosar-U | Dabrafenib | Dacarbazine | Dacogen |
| (Cytarabine) | | | (Decitabine) |
| Dacomitinib | Dactinomycin | Daratumumab | Darbepoetin Alfa |
| Darzalex | Dasatinib | Daunorubicin | Daunorubicin |
| (Daratumumab) | | Hydrochloride | Hydrochloride |
| | | | and Cytarabine |
| | | | Liposome |
| Decitabine | Defibrotide | Defitelio | Degarelix |
| | Sodium | (Defibrotide | |
| | | Sodium) | |
| Denileukin | Denosumab | DepoCyt | Dexamethasone |
| Diftitox | | (Cytarabine | |
| | | Liposome) | |
| Dexrazoxane | Dinutuximab | Docetaxel | Doxil |
| Hydrochloride | | | (Doxorubicin |
| | | | Hydrochloride |
| | | | Liposome) |
| Doxorubicin | Doxorubicin | Dox-SL | Durvalumab |
| Hydrochloride | Hydrochloride | (Doxorubicin | |
| | Liposome | Hydrochloride Liposome) | |
| Duvelisib | Efudex | Eligard | Elitek |
| | (Fluorouracil-- | (Leuprolide | (Rasburicase) |
| | Topical) | Acetate) | |
| Ellence | Elotuzumab | Eloxatin | Eltrombopag |
| (Epirubicin | | (Oxaliplatin) | Olamine |
| Hydrochloride) | | | |
| Emend | Empliciti | Enasidenib Mesylate | Encorafenib |
| (Aprepitant) | (Elotuzumab) | | |
| Enzalutamide | Epirubicin | EPOCH | Epoetin Alfa |
| | Hydrochloride | | |
| Epogen (Epoetin | Erbitux | Eribulin Mesylate | Erivedge |
| Alfa) | (Cetuximab) | | (Vismodegib) |
| Erleada | Erlotinib | Erwinaze | Ethyol |
| (Apalutamide) | Hydrochloride | (Asparaginase | (Amifostine) |
| | | Erwinia chrysanthemi) | |
| Etopophos | Etoposide | Etoposide Phosphate | Evacet |
| (Etoposide | | | (Doxorubicin |
| Phosphate) | | | Hydrochloride |
| | | | Liposome) |
| Everolimus | Evista | Evomela | Exemestane |
| | (Raloxifene Hydrochloride) | (Melphalan Hydrochloride) | |
| 5-FU | 5-FU | Fareston | Farydak |
| (Fluorouracil | (Fluorouracil-- | (Toremifene) | (Panobinostat |
| Injection) | Topical) | | lactate) |
| Faslodex | FEC | Femara | Filgrastim |
| (Fulvestrant) | | (Letrozole) | |
| Firmagon | Fludarabine | Fluoroplex | Fluorouracil |
| (Degarelix) | Phosphate | (Fluorouracil-- | Injection |
| | | Topical) | |
| Fluorouracil-- | Flutamide | FOLFIRI | FOLFIRI- |
| Topical | | | BEVACIZUMAB |
| FOLFIRI- | FOLFIRINOX | FOLFOX | Folotyn |
| CETUXIMAB | | | (Pralatrexate) |
| Fostamatinib | FU-LV | Fulvestrant | Fusilev |
| Disodium | | | (Leucovorin |
| | | | Calcium) |
| Gardasil | Gardasil 9 | Gazyva | Gefitinib |
| (Recombinant | (Recombinant | (Obinutuzumab) | |
| HPV Quadrivalent | HPV Nonavalent | | |
| Vaccine) | Vaccine) | | |
| Gemcitabine | GEMCITABINE- | GEMCITABINE- | Gemtuzumab |
| Hydrochloride | CISPLATIN | OXALIPLATIN | Ozogamicin |
| Gemzar | Gilotrif | Gleevec | Gliadel Wafer |
| (Gemcitabine | (Afatinib | (Imatinib | (Carmustine |
| Hydrochloride) | Dimaleate) | Mesylate) | Implant) |
| Glucarpidase | Goserelin Acetate | Granisetron | Granisetron |
| | | | Hydrochloride |
| Granix | Halaven | Hemangeol | Herceptin |
| (Filgrastim) | (Eribulin | (Propranolol | (Trastuzumab) |
| | Mesylate) | Hydrochloride) | |
| HPV Bivalent | HPV Nonavalent | HPV Quadrivalent | Hycamtin |
| Vaccine, | Vaccine, | Vaccine, Recombinant | (Topotecan |
| Recombinant | Recombinant | | Hydrochloride) |
| Hydrea | Hydroxyurea | Hyper-CVAD | Ibrance |
| (Hydroxyurea) | | | (Palbociclib) |
| Ibritumomab | Ibrutinib | ICE | Iclusig (Ponatinib |
| Tiuxetan | | | Hydrochloride) |
| Idarubicin | Idelalisib | Idhifa | Ifex (Ifosfamide) |
| Hydrochloride | | (Enasidenib | |
| | | Mesylate) | |
| Ifosfamide | IL-2 | Imatinib Mesylate | Imbruvica |
| | (Aldesleukin) | | (Ibrutinib) |
| Imfinzi | Imiquimod | Imlygic | Inlyta (Axitinib) |
| (Durvalumab) | | (Talimogene | |
| | | Laherparepvec) | |
| Inotuzumab | Interferon Alfa- | Interleukin-2 | Intron A |
| Ozogamicin | 2b, Recombinant | (Aldesleukin) | (Recombinant |
| | | | Interferon Alfa-2b) |
| Iobenguane I 131 | Ipilimumab | Iressa | Irinotecan |
| | | (Gefitinib) | Hydrochloride |
| Irinotecan | Istodax | Ivosidenib | Ixabepilone |
| Hydrochloride | (Romidepsin) | | |
| Liposome | | | |
| Ixazomib Citrate | Ixempra | Jakafi | JEB |
| | (Ixabepilone) | (Ruxolitinib | |
| | | Phosphate) | |
| Jevtana | Kadcyla (Ado- | Kepivance | Keytruda |
| (Cabazitaxel) | Trastuzumab | (Palifermin) | (Pembrolizumab) |
| | Emtansine) | | |
| Kisqali | Kymriah | Kyprolis | Lanreotide |
| (Ribociclib) | (Tisagenlecleucel) | (Carfilzomib) | Acetate |
| Lapatinib | Larotrectinib | Lartruvo | Lenalidomide |
| Ditosylate | Sulfate | (Olaratumab) | |
| Lenvatinib | Lenvima | Letrozole | Leucovorin |
| Mesylate | (Lenvatinib | | Calcium |
| | Mesylate) | | |
| Leukeran | Leuprolide | Levulan | Libtayo |
| (Chlorambucil) | Acetate | Kerastik | (Cemiplimab- |
| | | (Aminolevulinic Acid) | rwlc) |
| LipoDox | Lomustine | Lonsurf | Lorbrena |
| (Doxorubicin | | (Trifluridine | (Lorlatinib) |
| Hydrochloride | | and Tipiracil | |
| Liposome) | | Hydrochloride) | |
| Lorlatinib | Lumoxiti | Lupron | Lupron Depot |
| | (Moxetumomab | (Leuprolide | (Leuprolide |
| | Pasudotox-tdfk) | Acetate) | Acetate) |
| Lutathera | Lutetium | Lynparza | Marqibo |
| (Lutetium Lu 177- | (Lu 177- | (Olaparib) | (Vincristine |
| Dotatate) | Dotatate) | | Sulfate |
| | | | Liposome) |
| Matulane | Mechlorethamine | Megestrol Acetate | Mekinist |
| (Procarbazine | Hydrochloride | | (Trametinib) |
| Hydrochloride) | | | |
| Mektovi | Melphalan | Melphalan | Mercaptopurine |
| (Binimetinib) | | Hydrochloride | |
| Mesna | Mesnex | Methotrexate | Methylnaltrexone |
| | (Mesna) | | Bromide |
| Midostaurin | Mitomycin C | Mitoxantrone | Mogamulizumab- |
| | | Hydrochloride | kpkc |
| Moxetumomab | Mozobil | Mustargen | MVAC |
| Pasudotox-tdfk | (Plerixafor) | (Mechlorethamine | |
| | | Hydrochloride) | |
| Myleran | Mylotarg | Nanoparticle Paclitaxel | Navelbine |
| (Busulfan) | (Gemtuzumab | (Paclitaxel Albumin- | (Vinorelbine |
| | Ozogamicin) | stabilized Nanoparticle | Tartrate) |
| | | Formulation) | |
| Necitumumab | Nelarabine | Neratinib Maleate | Nerlynx |
| | | | (Neratinib |
| | | | Maleate) |
| Netupitant and | Neulasta | Neupogen | Nexavar |
| Palonosetron | (Pegfilgrastim) | (Filgrastim) | (Sorafenib |
| Hydrochloride | | | Tosylate) |
| Nilandron | Nilotinib | Nilutamide | Ninlaro |
| (Nilutamide) | | | (Ixazomib |
| | | | Citrate) |
| Niraparib Tosylate | Nivolumab | Nplate | Obinutuzumab |
| Monohydrate | | (Romiplostim) | |
| Odomzo | OEPA | Ofatumumab | OFF |
| (Sonidegib) | | | |
| Olaparib | Olaratumab | Omacetaxine | Oncaspar |
| | | Mepesuccinate | (Pegaspargase) |
| Ondansetron | Onivyde | Ontak | Opdivo |
| Hydrochloride | (Irinotecan | (Denileukin | (Nivolumab) |
| | Hydrochloride | Diftitox) | |
| | Liposome) | | |
| OPPA | Osimertinib | Oxaliplatin | Paclitaxel |
| Paclitaxel | PAD | Palbociclib | Palifermin |
| Albumin-stabilized | | | |
| Nanoparticle | | | |
| Formulation | | | |
| Palonosetron | Palonosetron | Pamidronate Disodium | Panitumumab |
| Hydrochloride | Hydrochloride | | |
| | and Netupitant | | |
| Panobinostat | Pazopanib | PCV | PEB |
| Lactate | Hydrochloride | | |
| Pegaspargase | Pegfilgrastim | Peginterferon Alfa-2b | PEG-Intron |
| | | | (Peginterferon |
| | | | Alfa-2b) |
| Pembrolizumab | Pemetrexed | Perjeta | Pertuzumab |
| | Disodium | (Pertuzumab) | |
| Plerixafor | Pomalidomide | Pomalyst | Ponatinib |
| | | (Pomalidomide) | Hydrochloride |
| Portrazza | Poteligeo | Pralatrexate | Prednisone |
| (Necitumumab) | (Mogamulizumab- | | |
| | kpkc) | | |
| Procarbazine | Procrit | Proleukin | Prolia |
| Hydrochloride | (Epoetin Alfa) | (Aldesleukin) | (Denosumab) |
| Promacta | Propranolol | Provenge | Purinethol |
| (Eltrombopag | Hydrochloride | (Sipuleucel-T) | (Mercaptopurine) |
| Olamine) | | | |
| Purixan | Radium 223 | Raloxifene | Ramucirumab |
| (Mercaptopurine) | Dichloride | Hydrochloride | |
| Rasburicase | R-CHOP | R-CVP | Recombinant |
| | | | Human |
| | | | Papillomavirus |
| | | | (HPV) Bivalent |
| | | | Vaccine |
| Recombinant | Recombinant | Recombinant Interferon | Regorafenib |
| Human | Human | Alfa-2b | |
| Papillomavirus | Papillomavirus | | |
| (HPV) Nonavalent | (HPV) | | |
| Vaccine | Quadrivalent | | |
| | Vaccine | | |
| Relistor | R-EPOCH | Retacrit | Revlimid |
| (Methylnaltrexone | | (Epoetin Alfa) | (Lenalidomide) |
| Bromide) | | | |
| Rheumatrex | Ribociclib | R-ICE | Rituxan |
| (Methotrexate) | | | (Rituximab) |
| Rituxan Hycela | Rituximab | Rituximab and | Rolapitant |
| (Rituximab and | | Hyaluronidase Human | Hydrochloride |
| Hyaluronidase | | | |
| Human) | | | |
| Romidepsin | Romiplostim | Rubidomycin | Rubraca |
| | | (Daunorubicin | (Rucaparib |
| | | Hydrochloride) | Camsylate) |
| Rucaparib | Ruxolitinib | Rydapt | Sancuso |
| Camsylate | Phosphate | (Midostaurin) | (Granisetron) |
| Sclerosol | Siltuximab | Sipuleucel-T | Somatuline Depot |
| Intrapleural Aerosol | | | (Lanreotide |
| (Talc) | | | Acetate) |
| Sonidegib | Sorafenib | Sprycel | STANFORD V |
| | Tosylate | (Dasatinib) | |
| Sterile Talc Powder | Steritale | Stivarga | Sunitinib Malate |
| (Talc) | (Talc) | (Regorafenib) | |
| Sustol | Sutent | Sylatron | Sylvant |
| (Granisetron) | (Sunitinib Malate) | (Peginterferon Alfa-2b) | (Siltuximab) |
| Synribo | Tabloid | TAC | Tafinlar |
| (Omacetaxine | (Thioguanine) | | (Dabrafenib) |
| Mepesuccinate) | | | |
| Tagrisso | Talc | Talimogene | Tamoxifen |
| (Osimertinib) | | Laherparepvec | Citrate |
| Tarabine PFS | Tarceva | Targretin | Tasigna |
| (Cytarabine) | (Erlotinib Hydrochloride) | (Bexarotene) | (Nilotinib) |
| Tavalisse | Taxol | Taxotere | Tecentriq |
| (Fostamatinib | (Paclitaxel) | (Docetaxel) | (Atezolizumab) |
| Disodium) | | | |
| Temodar | Temozolomide | Temsirolimus | Thalidomide |
| (Temozolomide) | | | |
| Thalomid | Thioguanine | Thiotepa | Tibsovo |
| (Thalidomide) | | | (Ivosidenib) |
| Tisagenlecleucel | Tocilizumab | Tolak | Topotecan |
| | | (Fluorouracil-- | Hydrochloride |
| | | Topical) | |
| Toremifene | Torisel | Totect | TPF |
| | (Temsirolimus) | (Dexrazoxane | |
| | | Hydrochloride) | |
| Trabectedin | Trametinib | Trastuzumab | Treanda |
| | | | (Bendamustine |
| | | | Hydrochloride) |
| Trexall | Trifluridine and | Trisenox | Tykerb |
| (Methotrexate) | Tipiracil | (Arsenic | (Lapatinib |
| | Hydrochloride | Trioxide) | Ditosylate) |
| Unituxin | Uridine Triacetate | VAC | Valrubicin |
| (Dinutuximab) | | | |
| Valstar | Vandetanib | VAMP | Varubi |
| (Valrubicin) | | | (Rolapitant |
| | | | Hydrochloride) |
| Vectibix | VelP | Velcade | Vemurafenib |
| (Panitumumab) | | (Bortezomib) | |
| Venclexta | Venetoclax | Verzenio | Vidaza |
| (Venetoclax) | | (Abemaciclib) | (Azacitidine) |
| Vinblastine Sulfate | Vincristine | Vincristine Sulfate | Vinorelbine |
| | Sulfate | Liposome | Tartrate |
| VIP | Vismodegib | Vistogard | Vitrakvi |
| | | (Uridine | (Larotrectinib |
| | | Triacetate) | Sulfate) |
| Vizimpro | Voraxaze | Vorinostat | Votrient |
| (Dacomitinib) | (Glucarpidase) | | (Pazopanib |
| | | | Hydrochloride) |
| Vyxeos | Xalkori | Xeloda | XELIRI |
| (Daunorubicin | (Crizotinib) | (Capecitabine) | |
| Hydrochloride and | | | |
| Cytarabine | | | |
| Liposome) | | | |
| XELOX | Xgeva | Xofigo | Xtandi |
| | (Denosumab) | (Radium 223 | (Enzalutamide) |
| | | Dichloride) | |
| Yervoy | Yescarta | Yondelis | Zaltrap (Ziv- |
| (Ipilimumab) | (Axicabtagene | (Trabectedin) | Aflibercept) |
| | Ciloleucel) | | |
| Zarxio | Zejula | Zelboraf | Zevalin |
| (Filgrastim) | (Niraparib Tosylate | (Vemurafenib) | (Ibritumomab |
| | Monohydrate) | | Tiuxetan) |
| Zinecard | Ziv-Aflibercept | Zofran | Zoladex |
| (Dexrazoxane | | (Ondansetron | (Goserelin |
| Hydrochloride) | | Hydrochloride) | Acetate) |
| Zoledronic Acid | Zolinza | Zometa | Zydelig |
| | (Vorinostat) | (Zoledronic Acid) | (Idelalisib) |
| Zykadia | Zytiga | | |
| (Ceritinib) | (Abiraterone | | |
| | Acetate) | | |

For a more detailed description of anticancer agents and other optional therapeutic agents, those skilled in the art are referred to any number of instructive manuals including, but not limited to, the Physician's Desk Reference and to Goodman and Gilman's “Pharmaceutical Basis of Therapeutics” tenth edition, Eds. Hardman et al., 2002.

The above-mentioned second therapeutically active agents, one or more of which can be used in combination with a Compound of the Disclosure, are prepared and administered as described in the art.

Compounds of the Disclosure typically are administered in admixture with a pharmaceutical carrier selected with regard to the intended route of administration and standard pharmaceutical practice. Pharmaceutical compositions for use in accordance with the present disclosure are formulated in a conventional manner using one or more physiologically acceptable carriers comprising excipients and/or auxiliaries that facilitate processing of Compound of the Disclosure.

These pharmaceutical compositions can be manufactured, for example, by conventional mixing, dissolving, granulating, dragee-making, emulsifying, encapsulating, entrapping, or lyophilizing processes. Proper formulation is dependent upon the route of administration chosen. When a therapeutically effective amount of the Compound of the Disclosure is administered orally, the composition typically is in the form of a tablet, capsule, powder, solution, or elixir. When administered in tablet form, the composition additionally can contain a solid carrier, such as a gelatin or an adjuvant. The tablet, capsule, and powder contain about 0.01% to about 95%, and preferably from about 1% to about 50%, of a Compound of the Disclosure. When administered in liquid form, a liquid carrier, such as water, petroleum, or oils of animal or plant origin, can be added. The liquid form of the composition can further contain physiological saline solution, dextrose or other saccharide solutions, or glycols. When administered in liquid form, the composition contains about 0.1% to about 90%, and preferably about 1% to about 50%, by weight, of a Compound of the Disclosure.

When a therapeutically effective amount of a Compound of the Disclosure is administered by intravenous, cutaneous, or subcutaneous injection, the composition is in the form of a pyrogen-free, parenterally acceptable aqueous solution. The preparation of such parenterally acceptable solutions, having due regard to pH, isotonicity, stability, and the like, is within the skill in the art. A preferred composition for intravenous, cutaneous, or subcutaneous injection typically contains, an isotonic vehicle.

Compounds of the Disclosure can be readily combined with pharmaceutically acceptable carriers well-known in the art. Standard pharmaceutical carriers are described in Remington's Pharmaceutical Sciences, Mack Publishing Co., Easton, PA, 19th ed. 1995. Such carriers enable the active agents to be formulated as tablets, pills, dragees, capsules, liquids, gels, syrups, slurries, suspensions and the like, for oral ingestion by a patient to be treated. Pharmaceutical preparations for oral use can be obtained by adding the Compound of the Disclosure to a solid excipient, optionally grinding the resulting mixture, and processing the mixture of granules, after adding suitable auxiliaries, if desired, to obtain tablets or dragee cores.

Suitable excipients include fillers such as saccharides (for example, lactose, sucrose, mannitol or sorbitol), cellulose preparations, calcium phosphates (for example, tricalcium phosphate or calcium hydrogen phosphate), as well as binders such as starch paste (using, for example, maize starch, wheat starch, rice starch, or potato starch), gelatin, tragacanth, methyl cellulose, hydroxypropylmethylcellulose, sodium carboxymethylcellulose, and/or polyvinyl pyrrolidone. If desired, one or more disintegrating agents can be added, such as the above-mentioned starches and also carboxymethyl-starch, cross-linked polyvinyl pyrrolidone, agar, or alginic acid or a salt thereof, such as sodium alginate. Buffers and pH modifiers can also be added to stabilize the pharmaceutical composition.

Auxiliaries are typically flow-regulating agents and lubricants such as, for example, silica, talc, stearic acid or salts thereof (e.g., magnesium stearate or calcium stearate), and polyethylene glycol. Dragee cores are provided with suitable coatings that are resistant to gastric juices. For this purpose, concentrated saccharide solutions can be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate can be used. Dye stuffs or pigments can be added to the tablets or dragee coatings, for example, for identification or in order to characterize combinations of active compound doses.

Compound of the Disclosure can be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions, or emulsions in oily or aqueous vehicles, and can contain formulatory agents such as suspending, stabilizing, and/or dispersing agents.

Pharmaceutical compositions for parenteral administration include aqueous solutions of the active agent in water-soluble form. Additionally, suspensions of a Compound of the Disclosure can be prepared as appropriate oily injection suspensions. Suitable lipophilic solvents or vehicles include fatty oils or synthetic fatty acid esters. Aqueous injection suspensions can contain substances which increase the viscosity of the suspension. Optionally, the suspension also can contain suitable stabilizers or agents that increase the solubility of the compounds and allow for the preparation of highly concentrated solutions. Alternatively, a present composition can be in powder form for constitution with a suitable vehicle, e.g., sterile pyrogen-free water, before use.

Compounds of the Disclosure also can be formulated in rectal compositions, such as suppositories or retention enemas, e.g., containing conventional suppository bases. In addition to the formulations described previously, the Compound of the Disclosure also can be formulated as a depot preparation. Such long-acting formulations can be administered by implantation (for example, subcutaneously or intramuscularly) or by intramuscular injection. Thus, for example, the Compound of the Disclosure can be formulated with suitable polymeric or hydrophobic materials (for example, as an emulsion in an acceptable oil) or ion exchange resins.

In particular, the Compounds of the Disclosure can be administered orally, buccally, or sublingually in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. Compound of the Disclosure also can be injected parenterally, for example, intravenously, intramuscularly, subcutaneously, or intracoronarily. For parenteral administration, the Compound of the Disclosure are typically used in the form of a sterile aqueous solution which can contain other substances, for example, salts or monosaccharides, such as mannitol or glucose, to make the solution isotonic with blood.

The disclosure provides the following particular embodiments in connection with treating a disease in a subject with a Compound of the Disclosure.

Embodiment I. A method of treating a subject, the method comprising administering to the subject a therapeutically effective amount of a Compound of the Disclosure, wherein the subject has cancer, neurodegenerative disease, diabetes, cardiovascular disease, kidney disease, or liver disease.

Embodiment II. The method Embodiment I, wherein the subject has cancer, e.g., any one of more of the cancers of Table 2 or Table 3.

Embodiment III. The method of Embodiment II, wherein the cancer is prostate cancer or breast cancer.

Embodiment IV. The method of Embodiment II, wherein the cancer is lung cancer, e.g., NSCLC.

Embodiment V. The method of Embodiment II, wherein the cancer is prostate cancer, e.g., metastatic castration-resistant prostate cancer.

Embodiment VI. The method of any one of Embodiments I-V further comprising administering a therapeutically effective amount of a second therapeutic agent useful in the treatment of the disease or condition, e.g., an immune checkpoint inhibitor or other anticancer agent.

Embodiment VII. A pharmaceutical composition comprising a Compound of the Disclosure and a pharmaceutically acceptable excipient.

Embodiment VIII. The pharmaceutical composition of Embodiment VII for use in treating cancer, neurodegenerative disease, diabetes, cardiovascular disease, kidney disease, or liver disease.

Embodiment IX. The pharmaceutical composition of Embodiment VIII, wherein the cancer is prostate cancer or breast cancer.

Embodiment X. The pharmaceutical composition of Embodiment VIII, wherein the cancer is lung cancer, e.g., NSCLC.

Embodiment XI. The pharmaceutical composition of Embodiment VIII, wherein the cancer is prostate cancer, e.g., metastatic castration-resistant prostate cancer.

Embodiment XII. A Compound of the Disclosure for use in treatment of cancer, neurodegenerative disease, diabetes, cardiovascular disease, kidney disease, or liver disease.

Embodiment XIII. The compound of Embodiment XIII for use in treating cancer.

Embodiment XIV. The compound of Embodiment XIII, wherein the cancer is breast cancer or lung cancer, e.g., NSCLC.

Embodiment XV. The compound of Embodiment XIII, wherein the cancer is prostate cancer, e.g., metastatic castration-resistant prostate cancer.

Embodiment XVI. Use of a Compound of the Disclosure for the manufacture of a medicament for treatment of cancer, neurodegenerative disease, diabetes, cardiovascular disease, kidney disease, or liver disease.

Embodiment XVII. The use of Embodiment XVI for the treatment of cancer.

Embodiment XVIII. The use of Embodiment XVII, wherein the cancer is prostate cancer or breast cancer.

Embodiment XIV. The use of Embodiment XVII, wherein the cancer is lung cancer, e.g., NSCLC.

Embodiment XX. The use of Embodiment XVII, wherein the cancer is prostate cancer, e.g., metastatic castration-resistant prostate cancer.

Embodiment XXI. A method of reducing Nrf2 protein within a cell of a subject in need thereof, the method comprising administering to the subject a Compound of the Disclosure. In one embodiment, the Nrf2 protein is reduced by about 50% or less, e.g., 1%, about 2%, about 3%, about 4%, about 5%, about 10%, about 15%, about 20%, about 25%, about 30%, about 35%, about 40%, or about 45%. In one embodiment, the Nrf2 protein is reduced by about 51% or more, e.g., about 55%, about 60%, about 65%, about 70%, about 75%, about 80%, about 85%, about 90%, or about 95%.

Embodiment XXII. The method of Embodiments I or VI, wherein the subject has neurodegenerative disease.

Embodiment XXIII. The method of Embodiments I or VI, wherein the subject has diabetes.

Embodiment XXIV. The method of Embodiments I or VI, wherein the subject has cardiovascular disease.

Embodiment XXV. The method of Embodiments I or VI, wherein the subject has kidney disease.

Embodiment XXVI. The method of Embodiments I or VI, wherein the subject has liver disease.

Embodiment XXVII. The pharmaceutical composition of Embodiment VIII, wherein the subject has neurodegenerative disease.

Embodiment XXVIII. The pharmaceutical composition of Embodiment VIII, wherein the subject has diabetes.

Embodiment XXIX. The pharmaceutical composition of Embodiment VIII, wherein the subject has cardiovascular disease.

Embodiment XXX. The pharmaceutical composition of Embodiment VIII, wherein the subject has kidney disease.

Embodiment XXXI. The pharmaceutical composition of Embodiment VIII, wherein the subject has liver disease.

Embodiment XXXII. The compound of Embodiment XII, wherein the subject has neurodegenerative disease.

Embodiment XXXIII. The compound of Embodiment XII, wherein the subject has diabetes.

Embodiment XXXIV. The compound of Embodiment XII, wherein the subject has cardiovascular disease.

Embodiment XXXV. The compound of Embodiment XII, wherein the subject has kidney disease.

Embodiment XXXVI. The compound of Embodiment XII, wherein the subject has liver disease.

Embodiment XXXVII. The use of Embodiment XVI, wherein the subject has neurodegenerative disease.

Embodiment XXXVIII. The use of Embodiment XVI, wherein the subject has diabetes.

Embodiment XXXIX. The use of Embodiment XVI, wherein the subject has cardiovascular disease.

Embodiment XL. The use of Embodiment XVI, wherein the subject has kidney disease.

Embodiment XLI. The use of Embodiment XVI, wherein the subject has liver disease.

### III. Kits of the Disclosure

In another embodiment, the present disclosure provides kits which comprise a Compound of the Disclosure (or a composition comprising a Compound of the Disclosure) packaged in a manner that facilitates its use to practice methods of the present disclosure. In one embodiment, the kit includes a Compound of the Disclosure (or a composition comprising a Compound of the Disclosure) packaged in a container, such as a sealed bottle or vessel, with a label affixed to the container or included in the kit that describes use of the compound or composition to practice the method of the disclosure. In one embodiment, the compound or composition is packaged in a unit dosage form. The kit further can include a device suitable for administering the composition according to the intended route of administration.

### IV. Definitions

The term “a disease or condition wherein degradation of Nrf2 provides a benefit” and the like pertains to a disease or condition in which Nrf2 is important or necessary, e.g., for the onset, progress, expression of that disease or condition, or a disease or a condition which is known to be treated by a Nrf2 inhibitor or degrader. Examples of such diseases or conditions include, but are not limited to, cancer, neurodegenerative disease, diabetes, cardiovascular disease, kidney disease, or liver disease. Dodson et al., Annu Rev Pharmacol Toxicol 59:555-575 (2019). One of ordinary skill in the art is readily able to determine whether a compound treats a disease or condition mediated by an Nrf2 degrader for any particular cell type, for example, by assays which conveniently can be used to assess the activity of particular compounds.

The term “Nrf2 degrader” and the like refer to a heterobifunctional small molecule that degrades Nrf2 protein. Nrf2 degraders contain a first ligand which binds to Nrf2 protein, a second ligand for an E3 ligase system, and a chemical linker that tethers the first and second ligands. Representative Compounds of the Disclosure that degrade Nrf2 protein are disclosed in Table 1.

The term “second therapeutic agent” refers to a therapeutic agent different from a Compound of the Disclosure and that is known to treat the disease or condition of interest. For example, when a cancer is the disease or condition of interest, the second therapeutic agent can be a known chemotherapeutic drug, like taxol, or radiation, for example.

The term “disease” or “condition” denotes disturbances and/or anomalies that as a rule are regarded as being pathological conditions or functions, and that can manifest themselves in the form of particular signs, symptoms, and/or malfunctions. Compounds of the Disclosure are degraders of Nrf2 and can be used in treating or preventing diseases and conditions wherein degradation of Nrf2 provides a benefit.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. The term “treat” and synonyms contemplate administering a therapeutically effective amount of a Compound of the Disclosure to a subject in need of such treatment. The treatment can be orientated symptomatically, for example, to suppress symptoms. It can be effected over a short period, be oriented over a medium term, or can be a long-term treatment, for example within the context of a maintenance therapy.

As used herein, the terms “prevent,” “preventing,” and “prevention” refer to a method of preventing the onset of a disease or condition and/or its attendant symptoms or barring a subject from acquiring a disease. As used herein, “prevent,” “preventing,” and “prevention” also include delaying the onset of a disease and/or its attendant symptoms and reducing a subject's risk of acquiring a disease. The terms “prevent,” “preventing” and “prevention” may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition.

The term “therapeutically effective amount” or “effective dose” as used herein refers to an amount of the active ingredient(s) that is(are) sufficient, when administered by a method of the disclosure, to efficaciously deliver the active ingredient(s) for the treatment of condition or disease of interest to a subject in need thereof. In the case of a cancer or other proliferation disorder, the therapeutically effective amount of the agent may reduce (i.e., retard to some extent or stop) unwanted cellular proliferation; reduce the number of cancer cells; reduce the tumor size; inhibit (i.e., retard to some extent or stop) cancer cell infiltration into peripheral organs; inhibit (i.e., retard to some extent or stop) tumor metastasis; inhibit, to some extent, tumor growth; and/or relieve, to some extent, one or more of the symptoms associated with the cancer. To the extent the administered compound or composition prevents growth and/or kills existing cancer cells, it may be cytostatic and/or cytotoxic.

The term “container” means any receptacle and closure therefore suitable for storing, shipping, dispensing, and/or handling a pharmaceutical product.

The term “insert” means information accompanying a pharmaceutical product that provides a description of how to administer the product, along with the safety and efficacy data required to allow the physician, pharmacist, and patient to make an informed decision regarding use of the product. The package insert generally is regarded as the “label” for a pharmaceutical product.

“Concurrent administration,” “administered in combination,” “simultaneous administration,” and similar phrases mean that two or more agents are administered concurrently to the subject being treated. By “concurrently,” it is meant that each agent is administered either simultaneously or sequentially in any order at different points in time. However, if not administered simultaneously, it is meant that they are administered to a subject in a sequence and sufficiently close in time so as to provide the desired therapeutic effect and can act in concert. For example, a Compound of the Disclosure can be administered at the same time or sequentially in any order at different points in time as a second therapeutic agent. A Compound of the Disclosure and the second therapeutic agent can be administered separately, in any appropriate form and by any suitable route. When a Compound of the Disclosure and the second therapeutic agent are not administered concurrently, it is understood that they can be administered in any order to a subject in need thereof. For example, a Compound of the Disclosure can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent treatment modality (e.g., radiotherapy), to a subject in need thereof. In various embodiments, a Compound of the Disclosure and the second therapeutic agent are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In one embodiment, the components of the combination therapies are administered at about 1 minute to about 24 hours apart.

The use of the terms “a”, “an”, “the”, and similar referents in the context of describing the disclosure (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the disclosure and is not a limitation on the scope of the disclosure unless otherwise claimed. No language in the specification should be construed as indicating any non-claimed element as essential to the practice of the disclosure.

The term “halo” as used herein by itself or as part of another group refers to —Cl, —F, —Br, or —I.

The term “nitro” as used herein by itself or as part of another group refers to —NO₂.

The term “cyano” as used herein by itself or as part of another group refers to —CN.

The term “hydroxy” as herein used by itself or as part of another group refers to —OH.

The term “alkyl” as used herein by itself or as part of another group refers to a straight- or branched-chain aliphatic hydrocarbon containing one to twelve carbon atoms, i.e., a C₁-C₁₂ alkyl, or the number of carbon atoms designated, e.g., a C₁ alkyl such as methyl, a C₂ alkyl such as ethyl, etc. In one embodiment, the alkyl is a C₁-C₁₀ alkyl. In another embodiment, the alkyl is a C₁-C₆ alkyl. In another embodiment, the alkyl is a C₁-C₄ alkyl. In another embodiment, the alkyl is a C₁-C₃ alkyl, i.e., methyl, ethyl, propyl, or isopropyl. Non-limiting exemplary C₁-C₁₂ alkyl groups include methyl, ethyl, propyl, isopropyl, butyl, sec-butyl, tert-butyl, iso-butyl, 3-pentyl, hexyl, heptyl, octyl, nonyl, and decyl.

The term “optionally substituted alkyl” as used herein by itself or as part of another group refers to an alkyl group that is either unsubstituted or substituted with one, two, or three substituents, wherein each substituent is independently nitro, haloalkoxy, aryloxy, aralkyloxy, alkylthio, sulfonamido, alkylcarbonyl, arylcarbonyl, alkylsulfonyl, arylsulfonyl, ureido, guanidino, carbamate, carboxy, alkoxycarbonyl, carboxyalkyl, —N(R^{56a})C(═O)R^{56b}, —N(R^{56c})S(═O)₂R^{56d}, —C(═O)R⁵⁷, —S(═O)R^{56e}, or —S(═O)₂R⁵⁸; wherein:
R^{56a} is hydrogen or alkyl;
R^{56b} is alkyl, haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, optionally substituted C₆-C₁₀ aryl, or optionally substituted heteroaryl;
R^{56c} is hydrogen or alkyl;
R^{56d} is alkyl, haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, optionally substituted C₆-C₁₀ aryl, or optionally substituted heteroaryl;
R^{56e} is alkyl, haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, optionally substituted C₆-C₁₀ aryl, or optionally substituted heteroaryl;
R⁵⁷ is haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, or optionally substituted heteroaryl; and
R⁵⁸ is haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, or optionally substituted heteroaryl. Non-limiting exemplary optionally substituted alkyl groups include —CH(CO₂Me)CH₂CO₂Me and —CH(CH₃)CH₂N(H)C(═O)O(CH₃)₃.

The term “alkenyl” as used herein by itself or as part of another group refers to an alkyl group containing one, two, or three carbon-to-carbon double bonds. In one embodiment, the alkenyl group is a C₂-C₆ alkenyl group. In another embodiment, the alkenyl group is a C₂-C₄ alkenyl group. In another embodiment, the alkenyl group has one carbon-to-carbon double bond. Non-limiting exemplary alkenyl groups include ethenyl, propenyl, isopropenyl, butenyl, sec-butenyl, pentenyl, and hexenyl.

The term “optionally substituted alkenyl” as used herein by itself or as part of another refers to an alkenyl group that is either unsubstituted or substituted with one, two or three substituents, wherein each substituent is independently halo, nitro, cyano, hydroxy, amino (e.g., alkylamino, dialkylamino), haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted cycloalkyl, alkenyl, alkynyl, optionally substituted aryl, optionally substituted heteroaryl, or optionally substituted heterocyclo. Non-limiting exemplary optionally substituted alkenyl groups include —CH═CHPh.

The term “alkynyl” as used herein by itself or as part of another group refers to an alkyl group containing one, two, or three carbon-to-carbon triple bonds. In one embodiment, the alkynyl is a C₂-C₆ alkynyl. In another embodiment, the alkynyl is a C₂-C₄ alkynyl. In another embodiment, the alkynyl has one carbon-to-carbon triple bond. Non-limiting exemplary alkynyl groups include ethynyl, propynyl, butynyl, 2-butynyl, pentynyl, and hexynyl groups.

The term “optionally substituted alkynyl” as used herein by itself or as part of another group refers to an alkynyl group that is either unsubstituted or substituted with one, two or three substituents, wherein each substituent is independently halo, nitro, cyano, hydroxy, amino, e.g., alkylamino, dialkylamino, haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted cycloalkyl, alkenyl, alkynyl, optionally substituted aryl, optionally substituted heteroaryl, or optionally substituted heterocyclo. Non-limiting exemplary optionally substituted alkynyl groups include —C≡CPh and —CH(Ph)C≡CH.

The term “haloalkyl” as used herein by itself or as part of another group refers to an alkyl group substituted by one or more fluorine, chlorine, bromine, and/or iodine atoms. In one embodiment, the alkyl is substituted by one, two, or three fluorine and/or chlorine atoms. In another embodiment, the alkyl is substituted by one, two, or three fluorine atoms. In another embodiment, the alkyl is a C₁-C₆ alkyl. In another embodiment, the alkyl is a C₁-C₄ alkyl. In another embodiment, the alkyl group is a C₁ or C₂ alkyl. Non-limiting exemplary haloalkyl groups include fluoromethyl, difluoromethyl, trifluoromethyl, pentafluoroethyl, 1,1-difluoroethyl, 2,2-difluoroethyl, 2,2,2-trifluoroethyl, 3,3,3-trifluoropropyl, 4,4,4-trifluorobutyl, and trichloromethyl groups.

The terms “hydroxyalkyl” or “(hydroxy)alkyl” as used herein by themselves or as part of another group refer to an alkyl group substituted with one, two, or three hydroxy groups. In one embodiment, the alkyl is a C₁-C₆ alkyl. In another embodiment, the alkyl is a C₁-C₄ alkyl. In another embodiment, the alkyl is a Cr or C₂ alkyl. In another embodiment, the hydroxyalkyl is a monohydroxyalkyl group, i.e., substituted with one hydroxy group. In another embodiment, the hydroxyalkyl group is a dihydroxyalkyl group, i.e., substituted with two hydroxy groups. Non-limiting exemplary (hydroxyl)alkyl groups include hydroxymethyl, hydroxyethyl, hydroxypropyl and hydroxybutyl groups, such as 1-hydroxyethyl, 2-hydroxyethyl, 1,2-dihydroxyethyl, 2-hydroxypropyl, 3-hydroxypropyl, 3-hydroxybutyl, 4-hydroxybutyl, 2-hydroxy-1-methylpropyl, and 1,3-dihydroxyprop-2-yl.

The term “alkoxy” as used herein by itself or as part of another group refers to an alkyl group attached to a terminal oxygen atom. In one embodiment, the alkyl is a C₁-C₆ alkyl and resulting alkoxy is thus referred to as a “C₁-C₆ alkoxy.” In another embodiment, the alkyl is a C₁-C₄ alkyl group. Non-limiting exemplary alkoxy groups include methoxy, ethoxy, and tert-butoxy.

The term “haloalkoxy” as used herein by itself or as part of another group refers to a haloalkyl group attached to a terminal oxygen atom. In one embodiment, the haloalkyl group is a C₁-C₆ haloalkyl. In another embodiment, the haloalkyl group is a C₁-C₄ haloalkyl group. Non-limiting exemplary haloalkoxy groups include fluoromethoxy, difluoromethoxy, trifluoromethoxy, and 2,2,2-trifluoroethoxy.

The term “alkylthio” as used herein by itself or as part of another group refers to an alkyl group attached to a terminal sulfur atom. In one embodiment, the alkyl group is a C₁-C₄ alkyl group. Non-limiting exemplary alkylthio groups include —SCH₃, and —SCH₂CH₃.

The terms “alkoxyalkyl” or “(alkoxy)alkyl” as used herein by themselves or as part of another group refers to an alkyl group substituted with one alkoxy group. In one embodiment, the alkoxy is a C₁-C₆ alkoxy. In another embodiment, the alkoxy is a C₁-C₄ alkoxy. In another embodiment, the alkyl is a C₁-C₆ alkyl. In another embodiment, the alkyl is a C₁-C₄ alkyl. Non-limiting exemplary alkoxyalkyl groups include methoxymethyl, methoxyethyl, methoxypropyl, methoxybutyl, ethoxymethyl, ethoxyethyl, ethoxypropyl, ethoxybutyl, propoxymethyl, iso-propoxymethyl, propoxyethyl, propoxypropyl, butoxymethyl, tert-butoxymethyl, isobutoxymethyl, sec-butoxymethyl, and pentyloxymethyl.

The term “heteroalkyl” as used by itself or part of another group refers to unsubstituted straight- or branched-chain aliphatic hydrocarbons containing from three to twenty chain atoms, i.e., 3- to 20-membered heteroalkyl, or the number of chain atoms designated, wherein at least one —CH₂— is replaced with at least one of —O—, —N(H)—, —N(C₁-C₄ alkyl)-, or —S—. The —O—, —N(H)—, —N(C₁-C₄ alkyl)-, or —S— can independently be placed at any position of the aliphatic hydrocarbon chain so long as each —O—, —N(H)—, —N(C₁-C₄ alkyl)-, and —S— group is separated by at least two —CH₂— groups. In one embodiment, one —CH₂— group is replaced with one —O— group. In another embodiment, two —CH₂— groups are replaced with two —O— groups. In another embodiment, three —CH₂— groups are replaced with three —O— groups. In another embodiment, four —CH₂— groups are replaced with four —O— groups. In another embodiment, one —CH₂— group is replaced with one —NH— group. Non-limiting exemplary heteroalkyl groups include CH₂OCH₃, —CH₂OCH—₂CH₂CH₃, —CH₂CH₂CH₂OCH₃, —NHCH₂CH₂OCH₂CH₂OCH₂CH₃, —CH₂CH₂OCH-₂CH₂OCH₂CH₃, —CH₂CH₂OCH₂CH₂OCH₂CH₂OCH₂CH₃, and —NHCH₂CH₂CH₂CH₃.

The term “cycloalkyl” as used herein by itself or as part of another group refers to saturated and partially unsaturated, e.g., containing one or two double bonds, monocyclic, bicyclic, or tricyclic aliphatic hydrocarbons containing three to twelve carbon atoms, i.e., a C₃₋₁₂ cycloalkyl, or the number of carbons designated, e.g., a C₃ cycloalkyl such a cyclopropyl, a C₄ cycloalkyl such as cyclobutyl, etc. In one embodiment, the cycloalkyl is bicyclic, i.e., it has two rings. In another embodiment, the cycloalkyl is monocyclic, i.e., it has one ring. In another embodiment, the cycloalkyl is a C₃₋₈ cycloalkyl. In another embodiment, the cycloalkyl is a C₃₋₆ cycloalkyl, i.e., cyclopropyl, cyclobutyl, cyclopentyl, or cyclohexyl. In another embodiment, the cycloalkyl is a C₅ cycloalkyl, i.e., cyclopentyl or cyclopentenyl. In another embodiment, the cycloalkyl is a C₆ cycloalkyl, i.e., cyclohexyl or cyclohexenyl. Non-limiting exemplary C₃₋₁₂ cycloalkyl groups include cyclopropyl, cyclobutyl, cyclopentyl, cyclohexyl, cycloheptyl, cyclooctyl, norbornyl, decalin, adamantyl, cyclohexenyl, and spiro[3.3]heptane.

The term “optionally substituted cycloalkyl” as used herein by itself or as part of another group refers to a cycloalkyl group that is either unsubstituted or substituted with one, two, or three substituents, wherein each substituent is independently halo, nitro, cyano, hydroxy, amino (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R^{56a})C(═O)R^{56b}, —N(R^{56c})S(═O)₂R^{56d}, —C(═O)R⁵⁷, —S(═O)R^{56e}, —S(═O)₂R⁵⁸, or —OR⁵⁹, wherein R^{56a}, R^{56b}, R^{56c}, R^{56d}, R^{56e}, R⁵⁷, and R⁵⁸ are as defined in connection with the term “optionally substituted alkyl” and R⁵⁹ is (hydroxy)alkyl or (amino)alkyl. The term optionally substituted cycloalkyl also includes cycloalkyl groups having fused optionally substituted aryl or optionally substituted heteroaryl groups such as

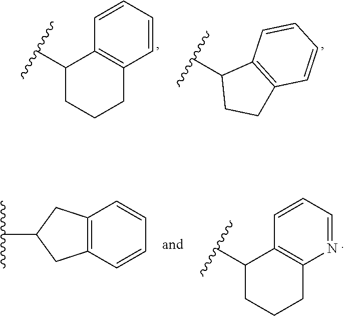

The term “heterocyclo” as used herein by itself or as part of another group refers to saturated and partially unsaturated, e.g., containing one or two double bonds, monocyclic, bicyclic, or tricyclic groups containing three to eighteen ring members, i.e., a 3- to 18-membered heterocyclo, comprising one, two, three, or four heteroatoms. Each heteroatom is independently oxygen, sulfur, or nitrogen. Each sulfur atom is independently oxidized to give a sulfoxide, i.e., S(═O), or sulfone, i.e., S(═O)₂. The term heterocyclo includes groups wherein one or more —CH₂— groups is replaced with one or more —C(═O)— groups, including cyclic ureido groups such as imidazolidinyl-2-one, cyclic amide groups such as pyrrolidin-2-one or piperidin-2-one, and cyclic carbamate groups such as oxazolidinyl-2-one. The term heterocyclo also includes groups having fused optionally substituted aryl or optionally substituted heteroaryl groups such as indoline, indolin-2-one, 2,3-dihydro-1H-pyrrolo[2,3-c]pyridine, 2,3,4,5-tetrahydro-1H-benzo[d]azepine, or 1,3,4,5-tetrahydro-2H-benzo[d]azepin-2-one.

In one embodiment, the heterocyclo group is a 4- to 8-membered cyclic group containing one ring and one or two oxygen atoms, e.g., tetrahydrofuran or tetrahydropyran, or one or two nitrogen atoms, e.g., pyrrolidine, piperidine, or piperazine, or one oxygen and one nitrogen atom, e.g., morpholine, and, optionally, one —CH₂— group is replaced with one —C(═O)— group, e.g., pyrrolidin-2-one or piperazin-2-one. In another embodiment, the heterocyclo group is a 5- to 8-membered cyclic group containing one ring and one or two nitrogen atoms and, optionally, one —CH₂— group is replaced with one —C(═O)— group. In another embodiment, the heterocyclo group is a 5- or 6-membered cyclic group containing one ring and one or two nitrogen atoms and, optionally, one —CH₂— group is replaced with one —C(═O)— group. In another embodiment, the heterocyclo group is a 8- to 12-membered cyclic group containing two rings and one or two nitrogen atoms. The heterocyclo can be linked to the rest of the molecule through any available carbon or nitrogen atom. Non-limiting exemplary heterocyclo groups include:

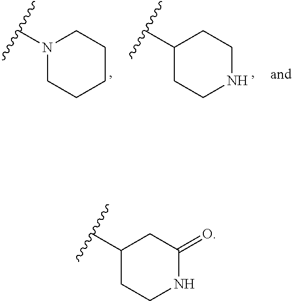

The term “optionally substituted heterocyclo” as used herein by itself or part of another group refers to a heterocyclo group that is either unsubstituted or substituted with one to four substituents, wherein each substituent is independently halo, nitro, cyano, hydroxy, amino, (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R^{56a})C(═O)R^{56b}, —N(R^{56c})S(═O)₂R^{56d}, —C(═O)R⁵⁷, —S(═O)R^{56e}, —S(═O)₂R⁵⁸, or —OR⁵⁹, wherein R^{56a}, R^{56b}, R^{56c}, R^{56d}, R^{56e}, R⁵⁷, R⁵⁸, and R⁵⁹ are as defined in connection with the term “optionally substituted cycloalkyl.” Substitution may occur on any available carbon or nitrogen atom of the heterocyclo group.

The term “aryl” as used herein by itself or as part of another group refers to an aromatic ring system having six to fourteen carbon atoms, i.e., C₆-C₁₄ aryl. Non-limiting exemplary aryl groups include phenyl (abbreviated as “Ph”), naphthyl, phenanthryl, anthracyl, indenyl, azulenyl, biphenyl, biphenylenyl, and fluorenyl groups. In one embodiment, the aryl group is phenyl or naphthyl. In another embodiment, the aryl group is phenyl.

The term “optionally substituted aryl” as used herein by itself or as part of another group refers to aryl that is either unsubstituted or substituted with one to five substituents, wherein the substituents are each independently halo, nitro, cyano, hydroxy, amino, (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R^{56a})C(═O)R^{56b}, —N(R^{56c})S(═O)₂R^{56d}, —C(═O)R⁵⁷, —S(═O)R^{56e}, —S(═O)₂R⁵⁸, or —OR⁵⁹, wherein R^{56a}, R^{56b}, R^{56c}, R^{56d}, R^{56e}, R⁵⁷, R⁵⁸, and R⁵⁹ are as defined in connection with the term “optionally substituted cycloalkyl.”

In one embodiment, the optionally substituted aryl is an optionally substituted phenyl. In another embodiment, the optionally substituted phenyl has four substituents. In another embodiment, the optionally substituted phenyl has three substituents. In another embodiment, the optionally substituted phenyl has two substituents. In another embodiment, the optionally substituted phenyl has one substituent. Non-limiting exemplary optionally substituted aryl groups include 2-methylphenyl, 2-methoxyphenyl, 2-fluorophenyl, 2-chlorophenyl, 2-bromophenyl, 3-methylphenyl, 3-methoxyphenyl, 3-fluorophenyl, 3-chlorophenyl, 4-methylphenyl, 4-ethylphenyl, 4-methoxyphenyl, 4-fluorophenyl, 4-chlorophenyl, 2,6-di-fluorophenyl, 2,6-di-chlorophenyl, 2-methyl, 3-methoxyphenyl, 2-ethyl, 3-methoxyphenyl, 3,4-di-methoxyphenyl, 3,5-di-fluorophenyl 3,5-di-methylphenyl, 3,5-dimethoxy, 4-methylphenyl, 2-fluoro-3-chlorophenyl, 3-chloro-4-fluorophenyl, and 2-phenylpropan-2-amine. The term optionally substituted aryl includes aryl groups having fused optionally substituted cycloalkyl groups and fused optionally substituted heterocyclo groups. Non-limiting examples include: 2,3-dihydro-1H-inden-1-yl, 1,2,3,4-tetrahydronaphthalen-1-yl, 1,3,4,5-tetrahydro-2H-benzo[c]azepin-2-yl, 1,2,3,4-tetrahydroisoquinolin-1-yl, and 2-oxo-2,3,4,5-tetrahydro-1H-benzo[d]azepin-1-yl.

The term “heteroaryl” as used herein by itself or as part of another group refers to monocyclic and bicyclic aromatic ring systems having five to 14 fourteen ring members, i.e., a 5- to 14-membered heteroaryl, comprising one, two, three, or four heteroatoms. Each heteroatom is independently oxygen, sulfur, or nitrogen. In one embodiment, the heteroaryl has three heteroatoms. In another embodiment, the heteroaryl has two heteroatoms. In another embodiment, the heteroaryl has one heteroatom. In another embodiment, the heteroaryl is a 5- to 10-membered heteroaryl. In another embodiment, the heteroaryl has 5 ring atoms, e.g., thienyl, a 5-membered heteroaryl having four carbon atoms and one sulfur atom. In another embodiment, the heteroaryl has 6 ring atoms, e.g., pyridyl, a 6-membered heteroaryl having five carbon atoms and one nitrogen atom. Non-limiting exemplary heteroaryl groups include thienyl, benzo[b]thienyl, naphtho[2,3-b]thienyl, thianthrenyl, furyl, benzofuryl, pyranyl, isobenzofuranyl, benzooxazonyl, chromenyl, xanthenyl, 2H-pyrrolyl, pyrrolyl, imidazolyl, pyrazolyl, pyridyl, pyrazinyl, pyrimidinyl, pyridazinyl, isoindolyl, 3H-indolyl, indolyl, indazolyl, purinyl, isoquinolyl, quinolyl, phthalazinyl, naphthyridinyl, cinnolinyl, quinazolinyl, pteridinyl, 4aH-carbazolyl, carbazolyl, β-carbolinyl, phenanthridinyl, acridinyl, pyrimidinyl, phenanthrolinyl, phenazinyl, thiazolyl, isothiazolyl, phenothiazolyl, isoxazolyl, furazanyl, and phenoxazinyl. In one embodiment, the heteroaryl is chosen from thienyl (e.g., thien-2-yl and thien-3-yl), furyl (e.g., 2-furyl and 3-furyl), pyrrolyl (e.g., 1H-pyrrol-2-yl and 1H-pyrrol-3-yl), imidazolyl (e.g., 2H-imidazol-2-yl and 2H-imidazol-4-yl), pyrazolyl (e.g., 1H-pyrazol-3-yl, 1H-pyrazol-4-yl, and 1H-pyrazol-5-yl), pyridyl (e.g., pyridin-2-yl, pyridin-3-yl, and pyridin-4-yl), pyrimidinyl (e.g., pyrimidin-2-yl, pyrimidin-4-yl, and pyrimidin-5-yl), thiazolyl (e.g., thiazol-2-yl, thiazol-4-yl, and thiazol-5-yl), isothiazolyl (e.g., isothiazol-3-yl, isothiazol-4-yl, and isothiazol-5-yl), oxazolyl (e.g., oxazol-2-yl, oxazol-4-yl, and oxazol-5-yl) and isoxazolyl (e.g., isoxazol-3-yl, isoxazol-4-yl, and isoxazol-5-yl). The term heteroaryl also includes N-oxides. A non-limiting exemplary N-oxide is pyridyl N-oxide.

The term “optionally substituted heteroaryl” as used herein by itself or as part of another group refers to a heteroaryl that is either unsubstituted or substituted with one to four substituents, wherein the substituents are independently halo, nitro, cyano, hydroxy, amino, (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R^{56a})C(═O)R^{56b}, —N(R^{56c})S(═O)₂R^{56d}, —C(═O)R⁵⁷, —S(═O)R^{56e}, —S(═O)₂R⁵⁸, or —OR⁵⁹, wherein R^{56a}, R^{56b}, R^{56c}, R^{56d}, R^{56e}, R⁵⁷, R⁵⁸, and R⁵⁹ are as defined in connection with the term “optionally substituted cycloalkyl.”

In one embodiment, the optionally substituted heteroaryl has two substituents. In another embodiment, the optionally substituted heteroaryl has one substituent. Any available carbon or nitrogen atom can be substituted.

The term “aryloxy” as used herein by itself or as part of another group refers to an optionally substituted aryl attached to a terminal oxygen atom. A non-limiting exemplary aryloxy group is PhO—.

The term “heteroaryloxy” as used herein by itself or as part of another group refers to an optionally substituted heteroaryl attached to a terminal oxygen atom. A non-limiting exemplary aryloxy group is pyridyl-O—.

The term “aralkyloxy” as used herein by itself or as part of another group refers to an aralkyl attached to a terminal oxygen atom. A non-limiting exemplary aralkyloxy group is PhCH₂O—.

The term “(cyano)alkyl” as used herein by itself or as part of another group refers to an alkyl substituted with one, two, or three cyano groups. In one embodiment, the alkyl is substituted with one cyano group. In another embodiment, the alkyl is a C₁-C₆ alkyl In another embodiment, the alkyl is a C₁-C₄ alkyl. Non-limiting exemplary (cyano)alkyl groups include —CH₂CH₂CN and —CH₂CH₂CH₂CN.

The term “(cycloalkyl)alkyl” as used herein by itself or as part of another group refers to an alkyl substituted with one or two optionally substituted cycloalkyl groups. In one embodiment, the cycloalkyl group(s) is an optionally substituted C₃-C₆ cycloalkyl. In another embodiment, the alkyl is a C₁-C₆ alkyl. In another embodiment, the alkyl is a C₁-C₄ alkyl. In another embodiment, the alkyl is a C₁ or C₂ alkyl. In another embodiment, the alkyl is substituted with one optionally substituted cycloalkyl group. In another embodiment, the alkyl is substituted with two optionally substituted cycloalkyl groups.

The term “sulfonamido” as used herein by itself or as part of another group refers to a radical of the formula —SO₂NR^{50a}R^{50b}, wherein R^{50a} and R^{50b} are each independently hydrogen, alkyl, optionally substituted cycloalkyl, optionally substituted heterocyclo, optionally substituted aryl, or optionally substituted heteroaryl; or R^{50a} and R^{50b} taken together with the nitrogen to which they are attached form a 3- to 8-membered optionally substituted heterocyclo group. Non-limiting exemplary sulfonamido groups include —SO₂NH₂, —SO₂N(H)CH₃, and —SO₂N(H)Ph.

The term “alkylcarbonyl” as used herein by itself or as part of another group refers to a carbonyl group, i.e., —C(═O)—, substituted by an alkyl group. In one embodiment, the alkyl is a C₁-C₄ alkyl. A non-limiting exemplary alkylcarbonyl group is —COCH₃.

The term “arylcarbonyl” as used herein by itself or as part of another group refers to a carbonyl group, i.e., —C(═O)—, substituted by an optionally substituted aryl group. A non-limiting exemplary arylcarbonyl group is —COPh.

The term “alkylsulfonyl” as used herein by itself or as part of another group refers to a sulfonyl group, i.e., —SO₂—, substituted by an alkyl group. A non-limiting exemplary alkylsulfonyl group is —SO₂CH₃.

The term “arylsulfonyl” as used herein by itself or as part of another group refers to a sulfonyl group, i.e., —SO₂—, substituted by an optionally substituted aryl group. A non-limiting exemplary arylsulfonyl group is —SO₂Ph.

The term “mercaptoalkyl” as used herein by itself or as part of another group refers to an alkyl substituted by a —SH group.

The term “carboxy” as used by itself or as part of another group refers to a radical of the formula —C(═O)OH.

The term “ureido” as used herein by itself or as part of another group refers to a radical of the formula —NR^{51a}—C(═O)—NR^{51b}R^{51c}, wherein R^{51a} is hydrogen or alkyl; and R^{51b} and R^{51c} are each independently hydrogen, alkyl, optionally substituted cycloalkyl, optionally substituted heterocyclo, optionally substituted aryl, or optionally substituted heteroaryl, or R^{51b} and R^{51c} taken together with the nitrogen to which they are attached form a 4- to 8-membered optionally substituted heterocyclo group. Non-limiting exemplary ureido groups include —NH—C(C═O)—NH₂ and —NH—C(C═O)—NHCH₃.

The term “guanidino” as used herein by itself or as part of another group refers to a radical of the formula —NR^{52a}—C(═NR⁵³)—NR^{52b}R^{52c}, wherein R^{52a} is hydrogen or alkyl; R^{52b} and R^{53c} are each independently hydrogen, alkyl, optionally substituted cycloalkyl, optionally substituted heterocyclo, optionally substituted aryl, or optionally substituted heteroaryl; or R^{52b} and R^{52c} taken together with the nitrogen to which they are attached form a 4- to 8-membered optionally substituted heterocyclo group; and R⁵³ is hydrogen, alkyl, cyano, alkylsulfonyl, alkylcarbonyl, carboxamido, or sulfonamido. Non-limiting exemplary guanidino groups include —NH—C(C═NH)—NH₂, —NH—C(C═NCN)—NH₂, and —NH—C(C═NH)—NHCH₃.

The term “(heterocyclo)alkyl” as used herein by itself or as part of another group refers to an alkyl substituted with one, two, or three optionally substituted heterocyclo groups. In one embodiment, the alkyl is substituted with one optionally substituted 5- to 8-membered heterocyclo group. In another embodiment, alkyl is a C₁-C₆ alkyl. In another embodiment, alkyl is a C₁-C₄ alkyl. The heterocyclo group can be linked to the alkyl group through a carbon or nitrogen atom.

The term “carbamate” as used herein by itself or as part of another group refers to a radical of the formula —NR^{54a}—C(═O)—OR^{54b}, wherein R^{54a} is hydrogen or alkyl, and R^{54b} is hydrogen, alkyl, optionally substituted cycloalkyl, optionally substituted heterocyclo, optionally substituted aryl, or optionally substituted heteroaryl. A non-limiting exemplary carbamate group is —NH—(C═O)—OtBu.

The term “(heteroaryl)alkyl” as used herein by itself or as part of another group refers to an alkyl substituted with one or two optionally substituted heteroaryl groups. In one embodiment, the alkyl group is substituted with one optionally substituted 5- to 14-membered heteroaryl group. In another embodiment, the alkyl group is substituted with two optionally substituted 5- to 14-membered heteroaryl groups. In another embodiment, the alkyl group is substituted with one optionally substituted 5- to 9-membered heteroaryl group. In another embodiment, the alkyl group is substituted with two optionally substituted 5- to 9-membered heteroaryl groups. In another embodiment, the alkyl group is substituted with one optionally substituted 5- or 6-membered heteroaryl group. In another embodiment, the alkyl group is substituted with two optionally substituted 5- or 6-membered heteroaryl groups. In one embodiment, the alkyl group is a C₁-C₆ alkyl. In another embodiment, the alkyl group is a C₁-C₄ alkyl. In another embodiment, the alkyl group is a C₁ or C₂ alkyl.

The terms “aralkyl” or “(aryl)alkyl” as used herein by themselves or as part of another group refers to an alkyl substituted with one, two, or three optionally substituted aryl groups. In one embodiment, the alkyl is substituted with one optionally substituted aryl group. In another embodiment, the alkyl is substituted with two optionally substituted aryl groups. In one embodiment, the aryl is an optionally substituted phenyl or optionally substituted naphthyl. In another embodiment, the aryl is an optionally substituted phenyl. In one embodiment, the alkyl is a C₁-C₆ alkyl. In another embodiment, the alkyl is a C₁-C₄ alkyl. In another embodiment, the alkyl is a C₁ or C₂ alkyl. Non-limiting exemplary (aryl)alkyl groups include benzyl, phenethyl, —CHPh₂, and —CH(4-F-Ph)₂.

The term “amido” as used herein by itself or as part of another group refers to a radical of formula —C(═O)NR^{60a}R^{60b}, wherein R^{60a} and R^{60b} are each independently hydrogen, optionally substituted alkyl, optionally substituted alkenyl, optionally substituted alkynyl, haloalkyl, (alkoxy)alkyl, (hydroxy)alkyl, (cyano)alkyl, optionally substituted cycloalkyl, optionally substituted heterocyclo, optionally substituted aryl, optionally substituted heteroaryl, (aryl)alkyl, (cycloalkyl)alkyl, (heterocyclo)alkyl, or (heteroaryl)alkyl; or R^{60a} and R^{60b} taken together with the nitrogen to which they are attached from a 4- to 8-membered optionally substituted heterocyclo group. In one embodiment, R^{60a} and R^{60b} are each independently hydrogen or C₁-C₆ alkyl.

The term “amino” as used by itself or as part of another group refers to a radical of the formula —NR^{55a}R^{55b}, wherein R^{55a} and R^{55b} are independently hydrogen, optionally substituted alkyl, haloalkyl, (hydroxy)alkyl, (alkoxy)alkyl, (amino)alkyl, heteroalkyl, optionally substituted cycloalkyl, optionally substituted heterocyclo, optionally substituted aryl, optionally substituted heteroaryl, (aryl)alkyl, (cycloalkyl)alkyl, (heterocyclo)alkyl, or (heteroaryl)alkyl.

In one embodiment, the amino is —NH₂.

In another embodiment, the amino is an “alkylamino,” i.e., an amino group wherein R^{55a} is C₁₋₆ alkyl and R^{55b} is hydrogen. In one embodiment, R^{55a} is C₁-C₄ alkyl. Non-limiting exemplary alkylamino groups include —N(H)CH₃ and —N(H)CH₂CH₃.

In another embodiment, the amino is a “dialkylamino,” i.e., an amino group wherein R^{55a} and R^{55b} are each independently C₁₋₆ alkyl. In one embodiment, R^{55a} and R^{55b} are each independently C₁-C₄ alkyl. Non-limiting exemplary dialkylamino groups include —N(CH₃)₂ and —N(CH₃)CH₂CH(CH₃)₂.

In another embodiment, the amino is a “hydroxyalkylamino,” i.e., an amino group wherein R^{55a} is (hydroxyl)alkyl and R^{55b} is hydrogen or C₁-C₄ alkyl.

In another embodiment, the amino is a “cycloalkylamino,” i.e., an amino group wherein R^{55a} is optionally substituted cycloalkyl and R^{55b} is hydrogen or C₁-C₄ alkyl.

In another embodiment, the amino is a “aralkylamino,” i.e., an amino group wherein R^{55a} is aralkyl and R^{55b} is hydrogen or C₁-C₄ alkyl. Non-limiting exemplary aralkylamino groups include —N(H)CH₂Ph, —N(H)CHPh₂, and —N(CH₃)CH₂Ph.

The term “(amino)alkyl” as used herein by itself or as part of another group refers to an alkyl substituted with one amino group. In one embodiment, the amino group is —NH₂. In one embodiment, the amino group is an alkylamino. In another embodiment, the amino group is a dialkylamino. In another embodiment, the alkyl is a C₁-C₆ alkyl. In another embodiment, the alkyl is a C₁-C₄ alkyl. Non-limiting exemplary (amino)alkyl groups include —CH₂NH₂, CH₂CH₂N(H)CH₃, —CH₂CH₂N(CH₃)₂, CH₂N(H)cyclopropyl, —CH₂N(H)cyclobutyl, and —CH₂N(H)cyclohexyl, and —CH₂CH₂CH₂N(H)CH₂Ph and —CH₂CH₂CH₂N(H)CH₂(4-CF₃-Ph).

The present disclosure encompasses any of the Compounds of the Disclosure being isotopically-labelled (i.e., radiolabeled) by having one or more atoms replaced by an atom having a different atomic mass or mass number. Examples of isotopes that can be incorporated into the disclosed compounds include isotopes of hydrogen, carbon, nitrogen, oxygen, phosphorous, fluorine and chlorine, such as ²H (or deuterium (D)), ³H, ¹¹C, ¹³C, ¹⁴C, ¹⁵N, ¹⁸O, ¹⁷O, ³¹P, ³²P, ³⁵S, ¹⁸F, and ³⁶Cl, respectively, e.g., ³H, ¹¹C, and ¹⁴C. In one embodiment, provided is a composition wherein substantially all of the atoms at a position within the Compound of the Disclosure are replaced by an atom having a different atomic mass or mass number. In another embodiment, provided is a composition wherein a portion of the atoms at a position within the Compound of the disclosure are replaced, i.e., the Compound of the Disclosure is enriched at a position with an atom having a different atomic mass or mass number.” Isotopically-labelled Compounds of the Disclosure can be prepared by methods known in the art.

As noted above, Compounds of the Disclosure contain one or more asymmetric carbon atoms and may thus give rise to enantiomers, diastereomers, and other stereoisomeric forms. The present disclosure encompasses the use of all such possible forms, as well as their racemic and resolved forms and mixtures thereof. The individual enantiomers can be separated according to methods known in the art in view of the present disclosure. When the compounds described herein contain olefinic double bonds or other centers of geometric asymmetry, and unless specified otherwise, it is intended that they include both E and Z geometric isomers. All tautomers are also encompassed by the present disclosure. All conformational isomers, i.e., stereoisomers produced by rotation about a σ bond, are also encompassed by the present disclosure.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

The term “chiral center” or “asymmetric carbon atom” refers to a carbon atom to which four different groups are attached.

The terms “enantiomer” and “enantiomeric” refer to a molecule that cannot be superimposed on its mirror image and hence is optically active wherein the enantiomer rotates the plane of polarized light in one direction and its mirror image compound rotates the plane of polarized light in the opposite direction.

The term “racemic” refers to a mixture of equal parts of enantiomers and which mixture is optically inactive. In one embodiment, Compounds of the Disclosure are racemic.

The term “absolute configuration” refers to the spatial arrangement of the atoms of a chiral molecular entity (or group) and its stereochemical description, e.g., R or S.

The stereochemical terms and conventions used in the specification are meant to be consistent with those described in Pure & Appl. Chem 68:2193 (1996), unless otherwise indicated.

The term “enantiomeric excess” or “ee” refers to a measure for how much of one enantiomer is present compared to the other. For a mixture of R and S enantiomers, the percent enantiomeric excess is defined as |R−S|*100, where R and S are the respective mole or weight fractions of enantiomers in a mixture such that R+S=1. With knowledge of the optical rotation of a chiral substance, the percent enantiomeric excess is defined as ([α]_{obs}/[α]ₘₐₓ)*100, where [α]_{obs} is the optical rotation of the mixture of enantiomers and [α]ₘₐₓ is the optical rotation of the pure enantiomer. Determination of enantiomeric excess is possible using a variety of analytical techniques, including NMR spectroscopy, chiral column chromatography or optical polarimetry.

The term “about,” as used herein, includes the recited number ±10%. Thus, “about 10” means 9 to 11.

### V. Particular Embodiments

The disclosure provides the following particular embodiments.

Embodiment 1. A compound of Formula A-I

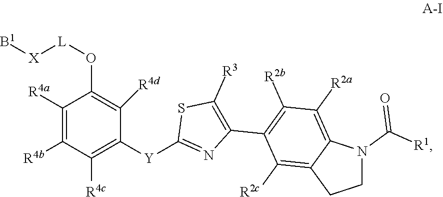

or a pharmaceutically acceptable salt or solvate thereof wherein:
   R¹ is selected from the group consisting of optionally substituted C₁-C₆ alkyl, optionally substituted C₃-C₈ cycloalkyl, optionally substituted phenyl, and optionally substituted 5- to 9-membered heteroaryl;
   R^{2a}, R^{2b}, and R^{2c} are independently selected from the group consisting of hydrogen, C₁-C₄ alkyl, C₁-C₄ haloalkyl, C₁-C₄ alkoxy, C₁-C₄ haloalkoxy, cyano, hydroxy, —S(═O)₂CH₃, and halo;
   R³ is selected from the group consisting of hydrogen, C₁-C₄ alkyl, C₁-C₄ haloalkyl, C₁-C₄ alkoxy, C₁-C₄ haloalkoxy, cyano, hydroxy, —S(═O)₂CH₃, and halo;
   Y is selected from the group consisting of —\hat{ }N(H)C(═O)—, —\hat{ }N(H)C(═O)CH₂—, —\hat{ }C(═O)N(H)— and —\hat{ }C(═O)N(H)CH₂—;
   wherein the bond marked with a “\hat{ }” is attached to the thiazole;
   R^{4a}, R^{4b}, R^{4c}, and R^{4d} are independently selected from the group consisting of hydrogen, C₁-C₄ alkyl, C₁-C₄ haloalkyl, C₁-C₄ alkoxy, C₁-C₄ haloalkoxy, cyano, hydroxy, —S(═O)₂CH₃, and halo;
   L is selected from the group consisting of —(CH₂)ₘ—, —*(CH₂)ₙ(OCH₂CH₂)ₒ—, and —(CH₂)ₚ—Z—CH₂)_{q}—;
   wherein the carbon marked with an “*” is attached to X;
   m is 2, 3, 4, 5, 6, 7, 8, 9, 10, 11, or 12;
   n is 2, 3, or 4;
   p is 0, 1, 2, 3, 4, 5, 6, 7, 8, 9, 10, 11, or 12;
   o is 1, 2, 3, 4, 5, 6, 7, 8, 9, 10, 11, or 12;
   q is 1, 2, 3, 4, 5, 6, 7, 8, 9, 10, 11, or 12;
   Z is selected from the group consisting of —(CR^{6a}R^{6b})— and —N(R⁷)—;
   R^{6a} is selected from the group consisting of halo, hydroxyl, C₁-C₆ alkyl, C₁-C₄ haloalkyl, optionally substituted C₃-C₅ cycloalkyl, substituted optionally C₄-C₈ heterocyclo, optionally substituted phenyl, and optionally substituted 5- to 9-membered heteroaryl;
   R^{6b} is selected from the group consisting of hydrogen and C₁-C₆ alkyl;
   R⁷ is selected from the group consisting of hydrogen, C₁-C₆ alkyl, and C₁-C₄ haloalkyl;
   X is selected from the group consisting of —O—, —NH—,

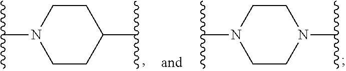 [SMILES: *C1CCN(*)CC1 ; *N1CCN(*)CC1]

B¹ is selected from the group consisting of:

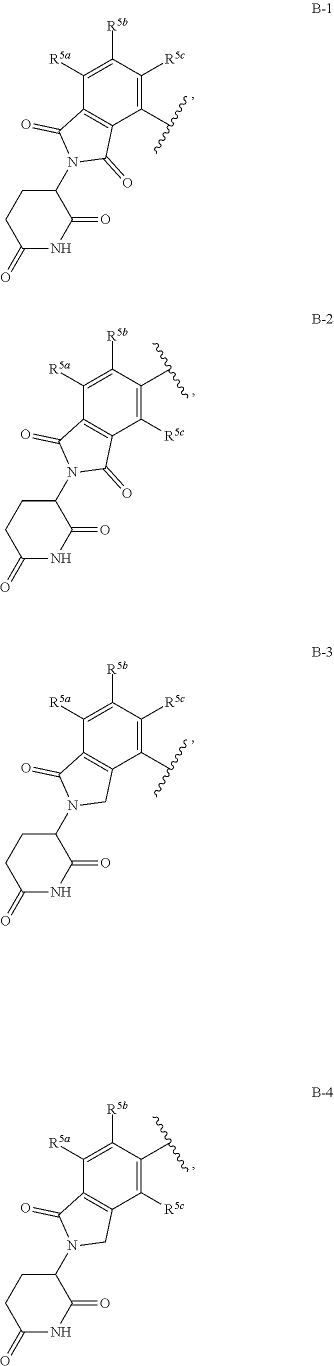

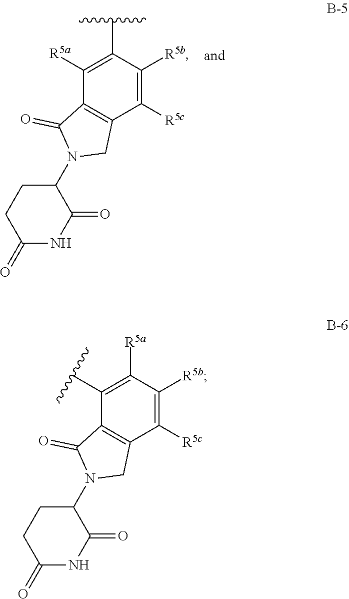

and
R^{5a}, R^{5b}, and R^{5c} are independently selected from the group consisting of hydrogen, C₁-C₄ alkyl, C₁-C₄ haloalkyl, C₁-C₄ alkoxy, C₁-C₄ haloalkoxy, cyano, hydroxy, —S(═O)₂CH₃, and halo.

Embodiment 2. A compound of Formula I:

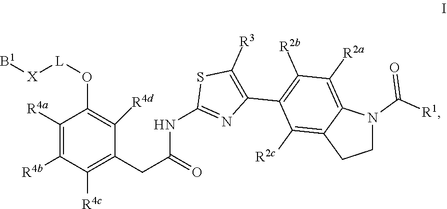

or a pharmaceutically acceptable salt or solvate thereof.

Embodiment 3. A compound of Formula II:

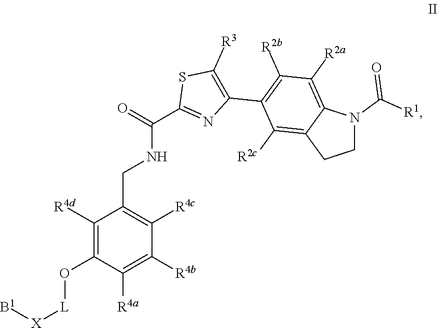

or a pharmaceutically acceptable salt or solvate thereof.

Embodiment 4. The compound of any of Embodiments 1-3, or a pharmaceutically acceptable salt or solvate thereof, wherein R¹ is optionally substituted C₃-C₈ cycloalkyl.

Embodiment 5. The compound of Embodiment 4, or a pharmaceutically acceptable salt or solvate thereof wherein R¹ is cyclopropyl.

Embodiment 6. The compound of any of Embodiments 1-3, or a pharmaceutically acceptable salt or solvate thereof, wherein R1 is optionally substituted phenyl. Embodiment 7. The compound of Embodiment 6, or a pharmaceutically acceptable salt or solvate thereof, wherein:
R¹ is:

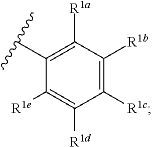

and
R^{1a}, R^{1b}, R^{1c}, R^{1d}, and R^{1e} are independently selected from the group consisting of hydrogen, C₁-C₄ alkyl, C₁-C₄ haloalkyl, C₁-C₄ alkoxy, C₁-C₄ haloalkoxy, cyano, hydroxy, —S(═O)₂CH₃, and halo.

Embodiment 8. The compound of Embodiment 7, or a pharmaceutically acceptable salt or solvate thereof, wherein R^{1b}, R^{1c}, R^{1d}, and R^{1e} are hydrogen.

Embodiment 9. The compound of Embodiments 7 or 8, or a pharmaceutically acceptable salt or solvate thereof, wherein R^{1a} is C₁-C₄ alkyl.

Embodiment 10. The compound of any of Embodiments 1-3, or a pharmaceutically acceptable salt or solvate thereof, wherein R¹ is optionally substituted 5- to 9-membered heteroaryl.

Embodiment 11. The compound of Embodiment 10, or a pharmaceutically acceptable salt or solvate thereof, wherein R¹ is optionally substituted imidazole.

Embodiment 12. The compound of any one of Embodiments 1-11, or a pharmaceutically acceptable salt or solvate thereof, wherein R^{2a}, R^{2b}, and R^{2c} are hydrogen.

Embodiment 13. The compound of any one of Embodiments 1-12, or a pharmaceutically acceptable salt or solvate thereof, wherein R³ is C₁-C₄ alkyl.

Embodiment 14. The compound of Embodiment 13, or a pharmaceutically acceptable salt or solvate thereof, wherein R³ is methyl.

Embodiment 15. The compound of any one of Embodiments 1-14, or a pharmaceutically acceptable salt or solvate thereof, wherein R^{4a} is selected from the group consisting of hydrogen and fluoro.

Embodiment 16. The compound of any one of Embodiments 1-15, or a pharmaceutically acceptable salt or solvate thereof, wherein R^{4b} is selected from the group consisting of hydrogen and fluoro.

Embodiment 17. The compound of any one of Embodiments 1-16, or a pharmaceutically acceptable salt or solvate thereof, wherein R^{4c} is selected from the group consisting of hydrogen and fluoro.

Embodiment 18. The compound of any one of Embodiments 1-14, or a pharmaceutically acceptable salt or solvate thereof, wherein R^{4b}, R^{4c}, and R^{4d} are hydrogen.

Embodiment 19. The compound of any one of Embodiments 1-18, or a pharmaceutically acceptable salt or solvate thereof, wherein L is selected from the group consisting of —(CH₂)ₘ— and —*(CH₂)ₙ(OCH₂CH₂)ₒ—.

Embodiment 20. The compound of Embodiment 19, or a pharmaceutically acceptable salt or solvate thereof, wherein L is —(CH₂)ₘ—.

Embodiment 21. The compound of Embodiment 19, or a pharmaceutically acceptable salt or solvate thereof wherein L is —*(CH₂)ₙ(OCH₂CH₂)ₒ—.

Embodiment 22. The compound of any one of Embodiments 1-18, or a pharmaceutically acceptable salt or solvate thereof wherein L is —(CH₂)ₚ—Z—CH₂)_{q}—.

Embodiment 23. The compound of Embodiment 22, or a pharmaceutically acceptable salt or solvate thereof, wherein Z is —(CR^{6a}R^{6b})—.

Embodiment 24. The compound of Embodiment 22, or a pharmaceutically acceptable salt or solvate thereof, wherein Z is —N(R′)—.

Embodiment 25. The compound of any one of Embodiments 1-24, or a pharmaceutically acceptable salt or solvate thereof, wherein X is —O—.

Embodiment 26. The compound of any one of Embodiments 1-24, or a pharmaceutically acceptable salt or solvate thereof, wherein X is —NH—.

Embodiment 27. The compound of any one of Embodiments 1-24, or a pharmaceutically acceptable salt or solvate thereof, wherein X is:

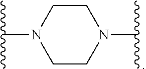 [SMILES: *N1CCN(*)CC1 ; "."]

Embodiment 28. The compound of any one of Embodiments 1-24, or a pharmaceutically acceptable salt or solvate thereof, wherein X is:

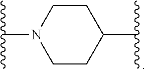 [SMILES: *C1CCN(*)CC1 ; "."]

Embodiment 29. The compound of any one of Embodiments 1-28, or a pharmaceutically acceptable salt or solvate thereof, wherein B¹ is B-1.

Embodiment 30. The compound of any one of Embodiments 1-28, or a pharmaceutically acceptable salt or solvate thereof, wherein B¹ is B-2.

Embodiment 31. The compound of any one of Embodiments 1-28, or a pharmaceutically acceptable salt or solvate thereof, wherein B¹ is B-3.

Embodiment 32. The compound of any one of Embodiments 1-28, or a pharmaceutically acceptable salt or solvate thereof, wherein B¹ is B-4.

Embodiment 33. The compound of any one of Embodiments 1-28, or a pharmaceutically acceptable salt or solvate thereof, wherein B¹ is B-5.

Embodiment 34. The compound of any one of Embodiments 1-28, or a pharmaceutically acceptable salt or solvate thereof, wherein B¹ is B-6.

Embodiment 35. The compound of any one of Embodiments 29-34, or a pharmaceutically acceptable salt or solvate thereof, wherein R^{5a}, R^{5b}, and R^{5c} are independently selected from the group consisting of hydrogen and fluoro.

Embodiment 36. The compound of Embodiment 35, or a pharmaceutically acceptable salt or solvate thereof, wherein R^{5a}, R^{5b}, and R^{5c} are hydrogen.

Embodiment 37. The compound of Embodiment 1, or a pharmaceutically acceptable salt or solvate thereof, wherein the compound is any one or more of the compounds in Table 1.

Embodiment 38. A pharmaceutical composition comprising the compound of any one of Embodiments 1-37, or a pharmaceutically acceptable salt thereof, and a pharmaceutically acceptable carrier.

Embodiment 39. A method of treating cancer, neurodegenerative disease, diabetes, cardiovascular disease, kidney disease, or liver disease in a subject in need thereof, the method comprising administering a therapeutically effective amount of the compound of any one of Embodiments 1-37, or a pharmaceutically acceptable salt thereof; to the subject.

Embodiment 40. The method of Embodiment 39 further comprising administering a second therapeutic agent to the subject.

Embodiment 41. The method of Embodiments 39 or 40 for treating cancer in a subject in need thereof.

Embodiment 42. The method of Embodiments 39 or 40, wherein the cancer is a solid tumor.

Embodiment 43. The method of Embodiments 39 or 40, wherein the cancer is a hematological cancer.

Embodiment 44. The method of Embodiments 39 or 40, wherein the cancer is one or more of the cancers of Table 2.

Embodiment 45. The method of any one of Embodiments 40-44, wherein the second therapeutic agent is isopropyl (S)-2-((S)-2-acetamido-3-(1H-indol-3-yl)propanamido)-6-diazo-5-oxohexanoate, or a pharmaceutically acceptable salt thereof.

Embodiment 46. A method of reducing nuclear factor erythroid 2-related factor 2 (Nrf2) protein within a cell of a subject, the method comprising administering to the subject a compound of any one of Embodiments 1-37, or a pharmaceutically acceptable salt or solvate thereof.

Embodiment 47. The method of Embodiment 46, wherein the subject has cancer.

Embodiment 48. A kit comprising the compound of any one of Embodiments 1-37 or a pharmaceutically acceptable salt thereof and instructions for administering the compound, or a pharmaceutically acceptable salt thereof, to a subject having cancer.

Embodiment 49. A compound of Formula III:

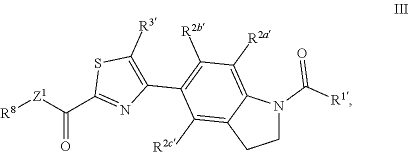

or a pharmaceutically acceptable salt thereof, wherein:
R^{1′} is selected from the group consisting of optionally substituted C₁-C₆ alkyl, optionally substituted C₃-C₈ cycloalkyl, optionally substituted phenyl, and optionally substituted 5- to 9-membered heteroaryl;
R^{2a′}, R^{2b′}, and R^{2c′} are independently selected from the group consisting of hydrogen, C₁-C₄ alkyl, C₁-C₄ haloalkyl, C₁-C₄ alkoxy, C₁-C₄ haloalkoxy, cyano, hydroxy, —S(═O)₂CH₃, and halo;
R^{3′} is selected from the group consisting of hydrogen, C₁-C₄ alkyl, C₁-C₄ haloalkyl, C₁-C₄ alkoxy, C₁-C₄ haloalkoxy, cyano, hydroxy, —S(═O)₂CH₃, and halo;
Z¹ is —O—; and
R⁸ is hydrogen, C₁-C₆ alkyl, or aralkyl; or
Z¹ is —N(H)—; and
R⁸ is:

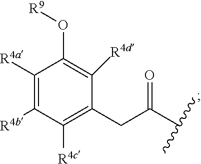

and
R^{4a′}, R^{4b′}, R^{4c′}, and R^{4d′} are independently selected from the group consisting of hydrogen, C₁-C₄ alkyl, C₁-C₄ haloalkyl, C₁-C₄ alkoxy, C₁-C₄ haloalkoxy, cyano, hydroxy, —S(═O)₂CH₃, and halo;
R⁹ is selected from the group consisting of hydrogen and -L¹-X¹.
X¹ is selected from the group consisting of —OR¹⁰ and —NR^{11a}R^{11b}.
R¹⁰ is hydrogen;
R^{11a} is selected from the group consisting of hydrogen and —C(═O)OtBu;
R^{11b} is selected from the group consisting of hydrogen and C₁-C₄ alkyl;
L¹ is selected from the group consisting of —(CH₂)_{m′}—, —*(CH)ₙ(OCH₂CH₂)_{o′}—, and —(CH₂)_{p′}—Z²—(CH₂)_{q′}—;
wherein the carbon marked with an “*” is attached to X¹;
m′ is 2, 3, 4, 5, 6, 7, 8, 9, 10, 11, or 12;
n′ is 2, 3, or 4;
o′ is 1, 2, 3, 4, 5, 6, 7, 8, 9, 10, 11, or 12;
p′ is 0, 1, 2, 3, 4, 5, 6, 7, 8, 9, 10, 11, or 12;
q′ is 1, 2, 3, 4, 5, 6, 7, 8, 9, 10, 11, or 12;
Z² is selected from the group consisting of —(CR^{6a′}—R^{6b′})— and —N(R⁷)—;
R^{6a′} is selected from the group consisting of halo, hydroxyl, C₁-C₆ alkyl, C₁-C₄ haloalkyl, optionally substituted C₃-C₈ cycloalkyl, substituted optionally C₄-C₈ heterocyclo, optionally substituted phenyl, and optionally substituted 5- to 9-membered heteroaryl;
R^{6b′} is selected from the group consisting of hydrogen and C₁-C₆ alkyl; and
R^{7′} is selected from the group consisting of hydrogen, C₁-C₆ alkyl, and C₁-C₄ haloalkyl.

### EXAMPLES

### Example 1

### Synthesis of 2-(3-hydroxyphenyl)-N-(5-methyl-4-(1-(2-methylbenzoyl)indolin-5-yl)thiazol-2-yl)acetamide (DCP_A017)

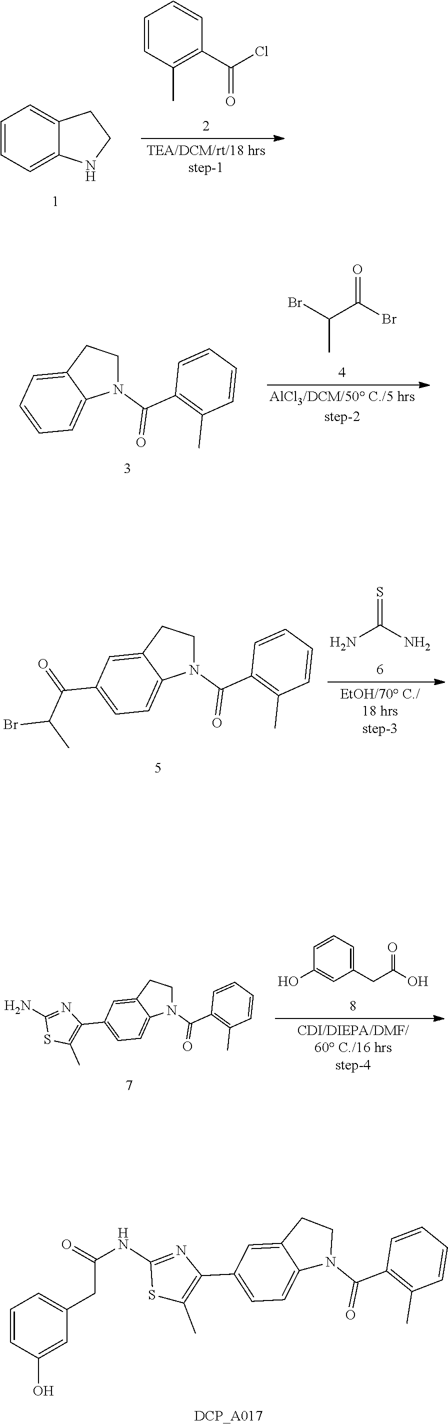

### Step 1. indolin-1-yl(o-tolyl)methanone

To a solution of indoline (20.00 g, 167.8 mmol), triethylamine (67.93 g, 671.3 mmol) in dichloromethane (500 mL), was added 2-methylbenzoyl chloride (25.94 g, 167.8 mmol) at 0° C. The mixture was stirred at room temperature for 18 hrs. The mixture was then quenched with water (200 mL), extracted with dichloromethane (500 mL×2), the organic layer washed with brine (100 mL), dried over anhydrous sodium sulfate, filtered and concentrated in vacuo. The residue was triturated with methyl tert-butyl ether (100 mL) to give indolin-1-yl(o-tolyl)methanone (28.0 g, 70.3% yield) as a yellow solid. MS (ESI) m/z 237.9 [M+H]⁺.

### Step 2. 2-bromo-1-(1-(2-methylbenzoyl)indolin-5-yl)propan-1-one

To a solution of indolin-1-yl(o-tolyl)methanone (28.00 g, 118.0 mmol) and aluminium chloride (47.20 g, 354.0 mmol) in dichloromethane (500 mL) at 0° C., was added 2-bromopropanoyl bromide (76.41 g, 354.0 mmol) dropwise. After addition, the mixture was stirred at 50° C. for 5 hrs. The mixture was then quenched with water (500 mL), basified to pH between 8-10 with 6N aqueous sodium hydroxide, extracted with dichloromethane (1.0 L), the organic layer dried over anhydrous sodium sulfate, filtered and concentrated in vacuo to give crude 2-bromo-1-(1-(2-methylbenzoyl)indolin-5-yl)propan-1-one (35.0 g, 79.7% yield) as a yellow oil. MS (ESI) m/z 372.0 and 374.0 [M+H]⁺.

### Step 3. (5-(2-amino-5-methylthiazol-4-yl)indolin-1-yl)(o-tolyl)methanone

A solution of 2-bromo-1-(1-(2-methylbenzoyl)indolin-5-yl)propan-1-one (35.00 g, 94.02 mmol) and thiourea (17.89 g, 235.1 mmol) in ethanol (500 mL) was stirred at 70° C. for 18 hrs. The solvent was removed in vacuo and the residue was purified by flash column chromatography (eluting with 100% dichloromethane) to give (5-(2-amino-5-methylthiazol-4-yl)indolin-1-yl)(o-tolyl)methanone (25.0 g, 76.1% yield) as a white solid. MS (ESI) m/z 349.9 [M+H]⁺.

### Step 4. 2-(3-hydroxyphenyl)-N-(5-methyl-4-(1-(2-methylbenzoyl)indolin-5-yl)thiazol-2-yl)acetamide

A solution of 2-(3-hydroxyphenyl)acetic acid (40 mg, 0.26 mmol), 1,1′-carbonyldiimidazole (64 mg, 0.39 mmol) and N,N-diisopropylethylamine (100 mg, 0.79 mmol) in N,N-dimethylformamide (2.5 mL) was stirred at room temperature for 30 min. Then (5-(2-amino-5-methylthiazol-4-yl)indolin-1-yl)(o-tolyl)methanone (101 mg, 0.29 mmol) was added into the resulting mixture at room temperature. The mixture was stirred at 60° C. for 16 hours at which time, the mixture was cooled to room temperature and directly purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15.0 min) to give 2-(3-hydroxyphenyl)-N-(5-methyl-4-(1-(2-methylbenzoyl)indolin-5-yl)thiazol-2-yl)acetamide (40 mg, 0.083 mmol, 31.4% yield) as white solid. ¹H NMR (400 MHz, DMSO-d₆): δ12.32 & 12.2* (s, 1H), 9.37 (s, 1H), 8.24 & 5.53* (d, J=8.4 Hz, 1H), 7.53 (s, 1H), 7.53, 7.49 & 7.04* (m, 1H), 7.37-7.32 (m, 4 H), 7.11 (t, J=13.6 Hz, 1H), 6.75-6.73 (m, 2H), 6.64 (d, J=7.6 Hz, 1H), 4.22 & 3.74* (m, 2H), 3.64 (s, 2H), 3.13 (m, 2H), 2.46 & 2.35* (s, 3H), 2.29 & 2.18* (s, 3H). MS (ESI) m/z 484.1 [M+H]⁺. *Multiple signals arising from conformational isomers.

### Example 2

### Synthesis of 2-(3-(2-(2-(2-(2,6-dioxopiperidin-3-yl)-1,3-dioxoisoindolin-4-ylamino)ethoxy)ethoxy)phenyl)-N-(5-methyl-4-(1-(2-methylbenzoyl)indolin-5-yl)thiazol-2-yl)acetamide (Cpd. No. 1)

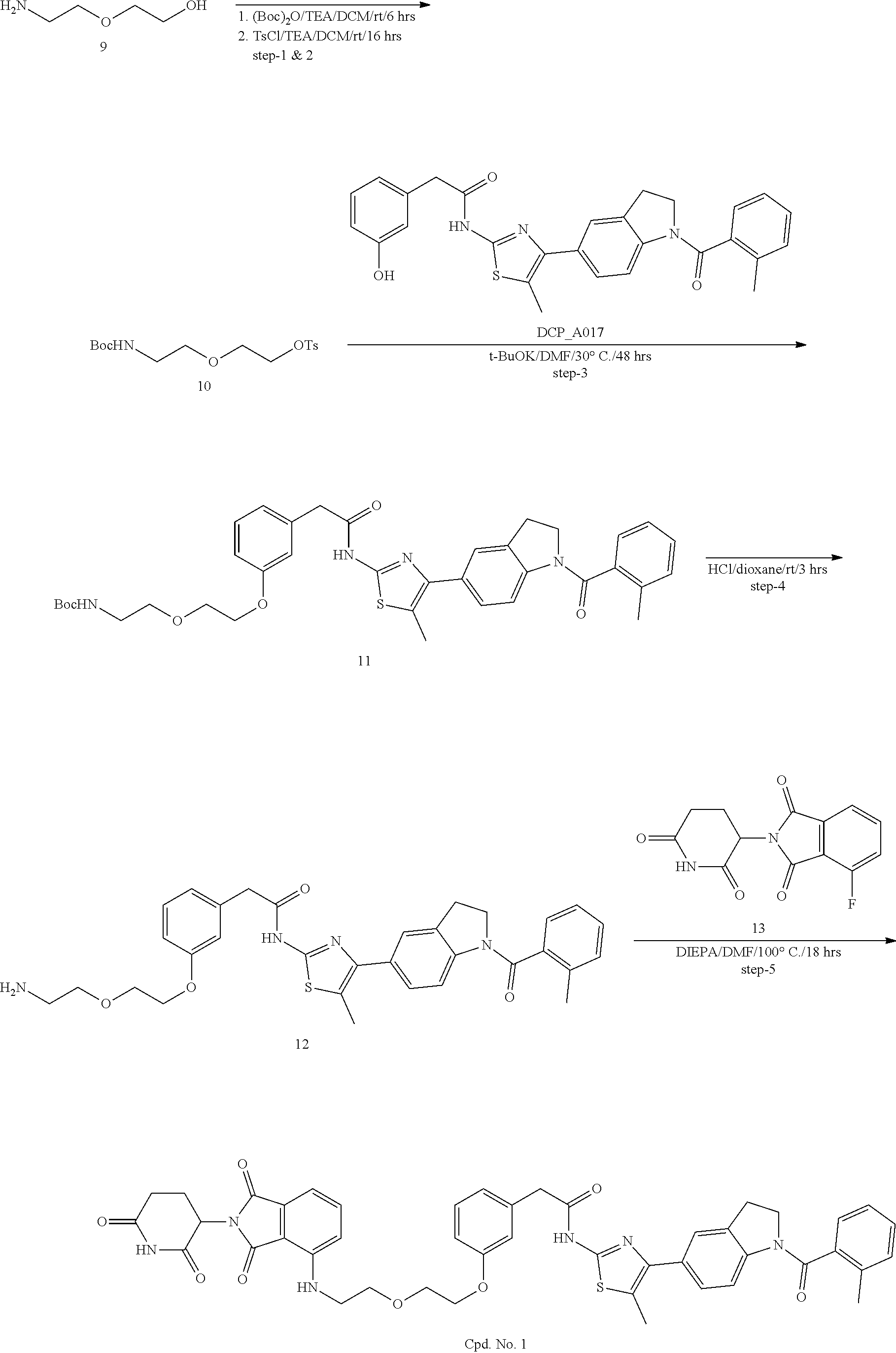

### Step 1. 2-(2-((tert-butoxycarbonyl)amino)ethoxy)ethyl 4-methylbenzenesulfonate

A solution of 2-(2-aminoethoxy)ethanol (1.00, 9.51 mmol), di-tert-butyl decarbonate (2.28 g, 10.5 mmol) and triethylamine (2.88 g, 28.5 mmol) in dichloromethane (50 mL) was stirred at room temperature for 6 hrs. Tosyl chloride (1.99 g, 10.5 mmol) was then added and the mixture was stirred for 16 hrs. the mixture was diluted with water (50 mL), extracted with dichloromethane (100 mL), the organic layer dried over anhydrous sodium sulfate, filtered and concentrated in vacuo. The residue was purified by flash column chromatography (eluting with dichloromethane) to give 2-(2-((tert-butoxycarbonyl)amino)ethoxy)ethyl 4-methylbenzenesulfonate (1.5 g, 43.8% yield) as a yellow oil. MS (ESI) m/z 260.1 [M+H−100]⁺.

### Step 3. tert-butyl (2-(2-(3-(2-((5-methyl-4-(1-(2-methylbenzoyl)indolin-5-yl)thiazol-2-yl)amino)-2-oxoethyl)phenoxy)ethoxy)ethyl)carbamate

A solution of 2-(3-hydroxyphenyl)-N-(5-methyl-4-(1-(2-methylbenzoyl)indolin-5-yl)thiazol-2-yl)acetamide (0.50 g, 1.03 mmol), 2-(2-((tert-butoxycarbonyl)amino)ethoxy)ethyl 4-methylbenzenesulfonate (0.37 g, 1.0 mmol), potassium tert-butoxide (0.35 g, 3.1 mmol) in N,N-dimethylformamide (25 mL) was stirred at 30° C. for 48 hrs. The mixture was then quenched with water (50 mL) and extracted with dichloromethane (50 mL×2). The organic layer was dried over anhydrous sodium sulfate, filtered and concentrated in vacuo. The crude residue was purified by flash column chromatography (eluting with ethyl acetate/petroleum ether=1/10) to give tert-butyl (2-(2-(3-(2-((5-methyl-4-(1-(2-methylbenzoyl)indolin-5-yl)thiazol-2-yl)amino)-2-oxoethyl)phenoxy)ethoxy)ethyl)carbamate (1.0 g, overweight) as an impure yellow oil. MS (ESI) m/z 671.3 [M+H]⁺.

### Step 4. 2-(3-(2-(2-aminoethoxy)ethoxy)phenyl)-N-(5-methyl-4-(1-(2-methylbenzoyl)indolin-5-yl)thiazol-2-yl)acetamide

A solution of tert-butyl (2-(2-(3-(2-((5-methyl-4-(1-(2-methylbenzoyl)indolin-5-yl)thiazol-2-yl)amino)-2-oxoethyl)phenoxy)ethoxy)ethyl)carbamate (1.00 g (impure), 1.0 mmol max) and 4 M HCl/1,4-dioxane solution (10.0 mL) in 1,4-dioxane (20.0 mL) was stirred at room temperature for 3 hrs. The solvent was removed in vacuo, the residue was dissolved in dichloromethane (100 mL), washed with saturated aqueous sodium carbonate, the organic layer dried over anhydrous sodium sulfate, filtered and concentrated in vacuo. The residue was purified by flash column chromatography (eluting with dichloromethane/methanol=5/100) to give 2-(3-(2-(2-aminoethoxy)ethoxy)phenyl)-N-(5-methyl-4-(1-(2-methylbenzoyl)indolin-5-yl)thiazol-2-yl)acetamide (90 mg, 16% overall yield for two steps) as a white solid. MS (ESI) m/z 571.3 [M+H]⁺.

### Step 5. 2-(3-(2-(2-((2-(2,6-dioxopiperidin-3-yl)-1,3-dioxoisoindolin-4-yl)amino)ethoxy)ethoxy)phenyl)-N-(5-methyl-4-(1-(2-methylbenzoyl)indolin-5-yl)thiazol-2-yl)acetamide

A solution of 2-(3-(2-(2-aminoethoxy)ethoxy)phenyl)-N-(5-methyl-4-(1-(2-methylbenzoyl)indolin-5-yl)thiazol-2-yl)acetamide (0.090 g, 0.16 mmol), 2-(2,6-dioxopiperidin-3-yl)-4-fluoroisoindoline-1,3-dione (0.046 g, 0.17 mmol) and N, N-Diisopropylethylamine (0.061 g, 0.47 mmol) in N,N-dimethylformamide (2.0 mL) was stirred at 100° C. for 18 hrs. The reaction mixture was directly purified by Prep-HPLC (Column: Welch Xtimate 21.2×250 mm C18, 10 μm, Mobile Phase: A: water (10 mmol/L ammonium bicarbonate) B: acetonitrile; B %: 30%-70% in 15.0 min) to give 2-(3-(2-(2-(2-(2,6-dioxopiperidin-3-yl)-1,3-dioxoisoindolin-4-ylamino)ethoxy)ethoxy)phenyl)-N-(5-methyl-4-(1-(2-methylbenzoyl)indolin-5-yl)thiazol-2-yl)acetamide (20.5 mg, 15.7% yield) as a yellow solid. ¹H NMR (400 MHz, DMSO-d₆): δ12.35 & 12.33 (s, 1H)*, 11.10 (s, 1H), 8.25 & 5.56* (d, J=8.8 Hz, 1H), 7.57-7.52 (m, 2H), 7.53, 7.46 & 7.04* (m, 1H), 7.37-7.34 (m, 7.23 (t, J=7.75 Hz, 1H), 7.15 (d, J=8.8 Hz, 1H), 7.03 (d, J=6.9 Hz, 1H), 6.93-6.89 (m, 2H), 6.84 (dd, J=8.2 & 0.95 Hz, 1H), 6.65 (t, J=6.7 Hz, 1H), 5.06 (dd, J=12.6 & 5.4 Hz, 1H), 4.22 & 3.70 (m, 2H)*, 4.10 (m, 2H), 3.80 (m, 2H), 3.78-3.65 (m, 3.51-3.47 (m, 2H), 3.18-3.12 (m, 2H), 2.92-2.84 (m, 1H), 2.60-2.53 (m, 2H), 2.46 & 2.35 (s, 3H)*, 2.30 & 2.18 (s, 3H)*, 2.02-1.99 (m, 1H). MS (ESI) m/z 827.5 [M+H]⁺ *Multiple signals arising from conformational isomers.

### Example 3

### Synthesis of 2-(3-(2-(2-((2-(2,6-dioxopiperidin-3-yl)-1,3-dioxoisoindolin-5-yl)amino)ethoxy)ethoxy)phenyl)-N-(5-methyl-4-(1-(2-methylbenzoyl)indolin-5-yl)thiazol-2-yl)acetamide (Cpd. No. 20)

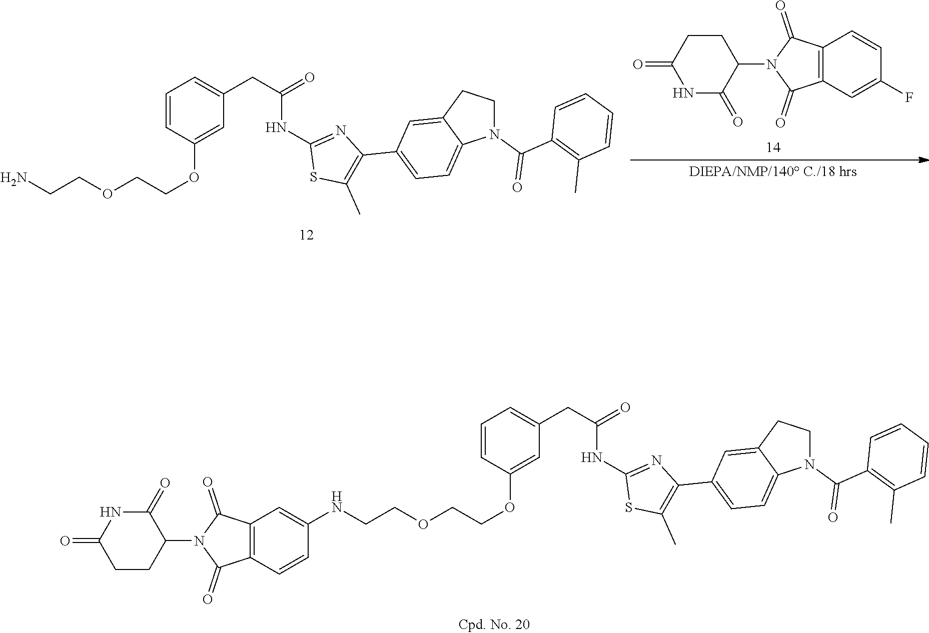 [SMILES: Cc1ccccc1C(=O)N1CCc2cc(-c3nc(NC(=O)Cc4cccc(OCCOCCN)c4)sc3C)ccc21 ; O=C1CCC(N2C(=O)c3ccc(F)cc3C2=O)C(=O)N1 ; Cc1ccccc1C(=O)N1CCc2cc(-c3nc(NC(=O)Cc4cccc(OCCOCCNc5ccc6c(c5)C(=O)N(C5CCC(=O)NC5=O)C6=O)c4)sc3C)ccc21]

### Step 1. 2-(3-(2-(2-((2-(2,6-dioxopiperidin-3-yl)-1,3-dioxoisoindolin-5-yl)amino)ethoxy)ethoxy)phenyl)-N-(5-methyl-4-(1-(2-methylbenzoyl)indolin-5-yl)thiazol-2-yl)acetamide

A solution of 2-(3-(2-(2-aminoethoxy)ethoxy)phenyl)-N-(5-methyl-4-(1-(2-methylbenzoyl)indolin-5-yl)thiazol-2-yl)acetamide (0.10 g, 0.18 mmol), 2-(2,6-dioxopiperidin-3-yl)-5-fluoroisoindoline-1,3-dione (0.048 g, 0.18 mmol) and N, N-diisopropylethylamine (0.068 g, 0.53 mmol) in 1-methylpyrrolidin-2-one (2.0 mL) was stirred at 140° C. for 18 hrs. The reaction mixture was then directly purified by Prep-HPLC (Column: Welch Xtimate 21.2×250 mm C18, 10 μm, Mobile Phase: A: water (10 mmol/L ammonium bicarbonate) B: acetonitrile; B %: 30%-70% in 15.0 min) to give 2-(3-(2-(2-((2-(2,6-dioxopiperidin-3-yl)-1,3-dioxoisoindolin-5-yl)amino)ethoxy)ethoxy)phenyl)-N-(5-methyl-4-(1-(2-methylbenzoyl)indolin-5-yl)thiazol-2-yl)acetamide (24.0 mg, 16% yield) as a yellow solid. ¹H1 NMR (400 MHz, DMSO-d₆): δ12.20 (brs, 1H), 11.09 (brs, 1H), 8.24 & 5.53* (d, J=8.8 Hz, 1H), 7.56-7.53 (m, 3H), 7.37-7.30 (m, 7.25-7.21 (m, 2H), 7.01 (s, 1H), 6.98-6.89 (m, 3H), 6.83 (d, J=7.6, 1H), 5.03 (dd, J=13.2 & 5.6 Hz, 1H), 4.22 & 3.70 (m, 2H)*, 4.10 (m, 2H), 3.78-3.60 (m, 6H), 3.94-3.80 (m, 2H), 3.13 (t, J=8.0 Hz, 2H), 2.89-2.83 (m, 1H), 2.59-2.55 (m, 2H), 2.45 & 2.34 (s, 3H)*, 2.30 & 2.18 (s, 3H)*, 2.29-2.03 (m, 1H). MS (ESI) m/z 827.5 [M+H]⁻. *Multiple signals arising from conformational isomers

### Example 4

### Synthesis of 2-(3-(2-(2-((2-(2,6-dioxopiperidin-3-yl)-1,3-dioxoisoindolin-4-yl)oxy)ethoxy)ethoxy)phenyl)-N-(5-methyl-4-(1-(2-methylbenzoyl)indolin-5-yl)thiazol-2-yl)acetamide (Cpd. No. 12)

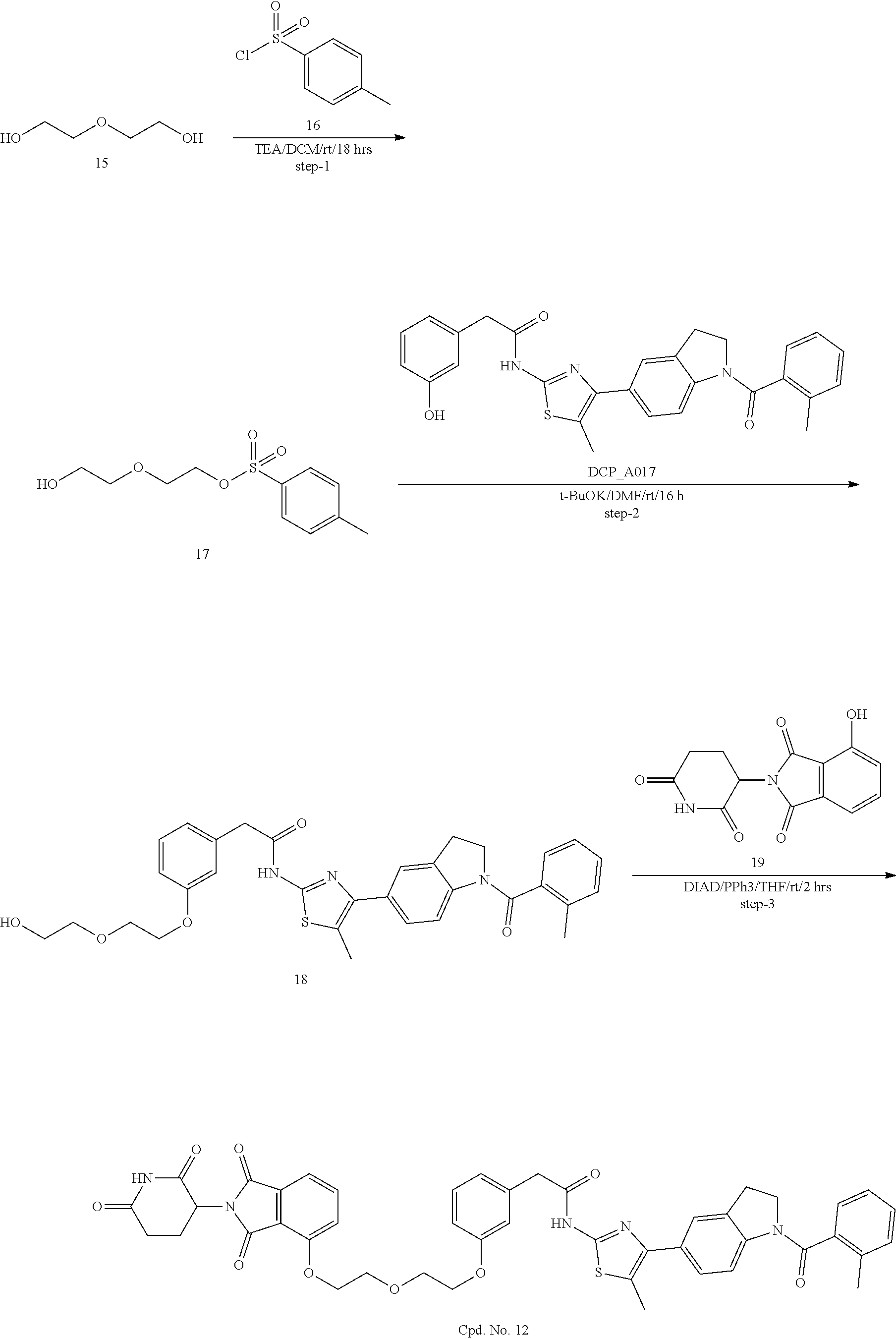

### Step 1. 2-(2-hydroxyethoxy)ethyl 4-mnethylbenzenesulfonate

A solution of 2,2′-oxybis(ethan-1-ol) (10.00 g, 94.23 mmol), 4-methylbenzenesulfonyl chloride (10.78 g, 56.54 mmol) and triethylamnine (28.61 g, 282.7 mmol) in dichloromethane (300 mL) was stirred at room temperature for 18 hrs. The solvent was removed and the residue was purified by flash column chromatography (ethyl acetate/petroleum ether=3/7) to give 2-(2-hydroxyethoxy)ethyl 4-methylbenzenesulfonate (8.0 g, 54% yield) as colorless oil. MS (ESI) m/z 161.0 [M+H]⁺.

### Step 2. 2-(3-(2-(2-hydroxyethoxy)ethoxy)phenyl)-N-(5-methyl-4-(1-(2-methylbenzoyl)indolin-5-yl)thiazol-2-yl)acetamide

A solution of 2-(2-hydroxyethoxy)ethyl 4-methylbenzenesulfonate (0.30 g, 1.15 mmol), 2-(3-hydroxyphenyl)-N-(4-(1-(2-methylbenzoyl)indolin-5-yl)thiazol-2-yl)acetamide (0.66 g, 1.38 mmol), potassium t-butoxide (0.39 g, 3.46 mmol) in N,N-dimethylformamide (10 mL) was stirred at room temperature for 16 hrs. The reaction mixture was quenched with water (80 mL), extracted with ethyl acetate (50 mL×2), washed with brine (20 mL×1), the organic layer dried over sodium sulfate, filtered and concentrated. The residue was purified by flash chromatography (petroleum ether/ethyl acetate=1/1) to give 2-(3-(2-(2-hydroxyethoxy)ethoxy)phenyl)-N-(5-methyl-4-(1-(2-methylbenzoyl)indolin-5-yl)thiazol-2-yl)acetamide (0.16 g, 24.3% yield) as yellow solid. MS (ESI) m/z 572.3 [M+H]⁺.

### Step 3. 2-(3-(2-(2-((2-(2,6-dioxopiperidin-3-yl)-1,3-dioxoisoindolin-4-yl)oxy)ethoxy)ethoxy)phenyl)-N-(5-methyl-4-(1-(2-methylbenzoyl)indolin-5-yl)thiazol-2-yl)acetamide

To a solution of 2-(3-(2-(2-hydroxyethoxy)ethoxy)phenyl)-N-(5-methyl-4-(1-(2-methylbenzoyl)indolin-5-yl)thiazol-2-yl)acetamide (0.16 g, 0.28 mmol), 2-(2,6-dioxopiperidin-3-yl)-4-hydroxyisoindoline-1,3-dione (0.092 g, 0.33 mmol) and triphenylphosphine (0.147 g, 0.56 mmol) in tetrahydrofuran (10 mL), was added diisopropyl azodicarboxylate (0.068 g, 0.33 mmol) at room temperature. The mixture was stirred at room temperature for 2 hrs. The mixture was diluted with water (30 mL), extracted with ethyl acetate (20 mL×2), the organic layer dried over anhydrous sodium sulfate, filtered and concentrated. The residue was purified by Prep-HPLC (Column: Welch Xtimate 21.2×250 mm C18, 10 μm, Mobile Phase: A: water (10 mmol/L ammonium bicarbonate) B: acetonitrile; B %: 30%-70% in 15.0 min) to give 2-(3-(2-(2-((2-(2,6-dioxopiperidin-3-yl)-1,3-dioxoisoindolin-4-yl)oxy)ethoxy)ethoxy)phenyl)-N-(5-methyl-4-(1-(2-methylbenzoyl)indolin-5-yl)thiazol-2-yl)acetamide (22 mg, 9.5% yield) as a white solid. ¹H NMR (400 MHz, DMSO-d₆): δ12.33 & 12.22 (s, 1H)*, 11.10 (s, 1 H), 8.23 & 5.56* (d, J=8.4 Hz, 1H), 7.79 (t, J=7.6 Hz, 1H), 7.54-7.52 (m, 2H), 7.54 &7.03* (m, 1H), 7.44 (d, J=7.6 Hz, 1H), 7.37-7.30 (m, 7.22 (t, J=8.0 Hz, 1H), 6.92-6.88 (m, 2H), 6.83 (d, J=8.4 Hz, 1H), 5.08 (dd, J=12.8 & 5.6 Hz, 1H), 4.37 (t, J=4.2 Hz, 2H), 4.21 & 3.74 (m, 2H)*, 4.10 (t, J=4.2 Hz, 2H), 3.89-3.87 (m, 3.76-3.67 (m, 23), 3.13 (t, J=7.8 Hz, 2H), 2.92-2.83 (m, 1H), 2.60-2.50 (m, 2H), 2.45 & 2.35 (s, 3H)*, 2.29 & 2.18 (s, 3H)*, 2.04-1.99 (m, 1H). MS (ESI) m/z 828.3 [M+H]⁺. *Multiple signals arising from conformational isomers.

### Example 5

### Synthesis of 2-(3-(2-(2-((2-(2,6-dioxopiperidin-3-yl)-1,3-dioxoisoindolin-5-yl)oxy)ethoxy)ethoxy)phenyl)-N-(5-methyl-4-(1-(2-methylbenzoyl)indolin-5-yl)thiazol-2-yl)acetamide (Cpd. No. 18)

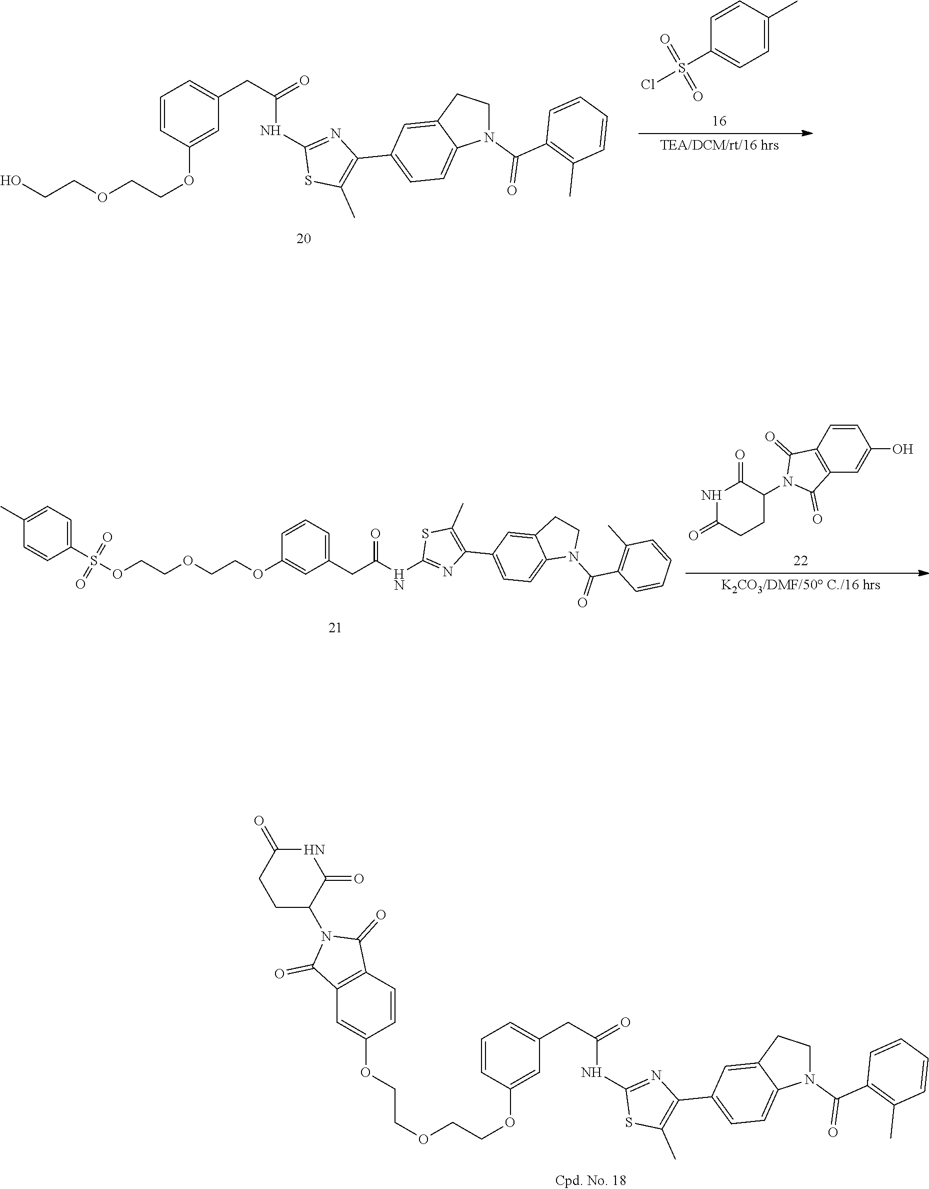

### Step 1. 2-(2-(3-(2-((5-methyl-4-(1-(2-methylbenzoyl)indolin-5-yl)thiazol-2-yl)amino)-2-oxoethyl)phenoxy)ethoxy)ethyl 4-methylbenzenesulfonate

A solution of 2-(3-(2-(2-hydroxyethoxy)ethoxy)phenyl)-N-(5-methyl-4-(1-(2-methylbenzoyl)indolin-5-yl)thiazol-2-yl)acetamide (120 mg, 0.21 mmol), tosyl chloride (59.3 mg, 0.31 mmol) and triethylamine (63.6 mg, 0.63 mmol) in dichloromethane (30 mL) was stirred at room temperature for 16 hrs. The mixture was diluted with water (10 mL), extracted with dichloromethane (30 mL), the organic layer was dried over anhydrous sodium sulfate, filtered and concentrated. The residue was purified by flash chromatography (dichloromethane) to give 2-(2-(3-(2-((5-methyl-4-(1-(2-methylbenzoyl)indolin-5-yl)thiazol-2-yl)amino)-2-oxoethyl)phenoxy)ethoxy)ethyl 4-methylbenzenesulfonate (80.0 mg, 52.6% yield) as a yellow oil. MS (ESI) m/z 726.3 [M+H]⁺.

### Step 2. 2-(3-(2-(2-((2-(2,6-dioxopiperidin-3-yl)-1,3-dioxoisoindolin-5-yl)oxy)ethoxy)ethoxy)phenyl)-N-(5-methyl-4-(1-(2-methylbenzoyl)indolin-5-yl)thiazol-2-yl)acetamide

A solution of 2-(2-(3-(2-((5-methyl-4-(1-(2-methylbenzoyl)indolin-5-yl)thiazol-2-yl)amino)-2-oxoethyl)phenoxy)ethoxy)ethyl 4-methylbenzenesulfonate (80.0 mg, 0.11 mmol), 2-(2,6-dioxopiperidin-3-yl)-5-hydroxyisoindoline-1,3-dione (60.2 mg, 0.22 mmol) and potassium carbonate (45.5 mg, 0.33 mmol) in N,N-dimethylformamide (10 mL) was stirred at 50° C. for 16 hrs. The reaction mixture was then directly purified by Prep-HPLC (Column: Welch Xtimate 21.2×250 mm C18, 10 μm, Mobile Phase: A: water (10 mmol/L ammonium bicarbonate) B: acetonitrile; B %: 30%-70% in 15.0 min) to give 2-(3-(2-(2-((2-(2,6-dioxopiperidin-3-yl)-1,3-dioxoisoindolin-5-yl)oxy)ethoxy)ethoxy)phenyl)-N-(5-methyl-4-(1-(2-methylbenzoyl)indolin-5-yl)thiazol-2-yl)acetamide (7.6 mg, 8.3% yield) as a white solid. ¹H NMR (400 MHz, DMSO-d₆): δ 12.33 (s, 1H), 11.12 (s, 1H), 8.23 & 5.45* (d, J=8.0 Hz, 1H), 7.81 (d, J=8.4 Hz, 1H), 7.52 (brs, 1H), 7.52 & 7.01* (brs, 1H), 7.45 (d, J=4.0 Hz, 1H), 7.35-7.32 (m, 5H), 7.23 (t, J=15.2 Hz, 1H), 6.92-6.89 (m, 2H), 6.84 (d, J=8 Hz, 1H), 5.11 (dd, J=14 & 6.0 Hz, 1H), 4.33 (m, 2H), 4.19 & 3.70 (m, 2H)*, 4.11 (m, 2H), 3.86-3.80 (m, 4H), 3.75-3.67 (m, 2H), 3.12 (t, J=8.0 Hz, 2H), 2.93-2.84 (m, 1H), 2.68-2.55 (m, 2H), 2.45 & 2.33 (s, 3H)*, 2.29 & 2.18 (s, 3H)*, 2.05-1.96 (m, 1H). MS (ESI) m/z 828.2 [M+H]⁺*Multiple signals arising from conformational isomers.

### Example 6

### Synthesis of 2-(3-(2-(2-(2-((2-(2,6-dioxopiperidin-3-yl)-1,3-dioxoisoindolin-4-yl)amino)ethoxy)ethoxy)ethoxy)phenyl)-N-(5-methyl-4-(1-(2-methylbenzoyl)indolin-5-yl)thiazol-2-yl)acetamide (Cpd. No. 2)

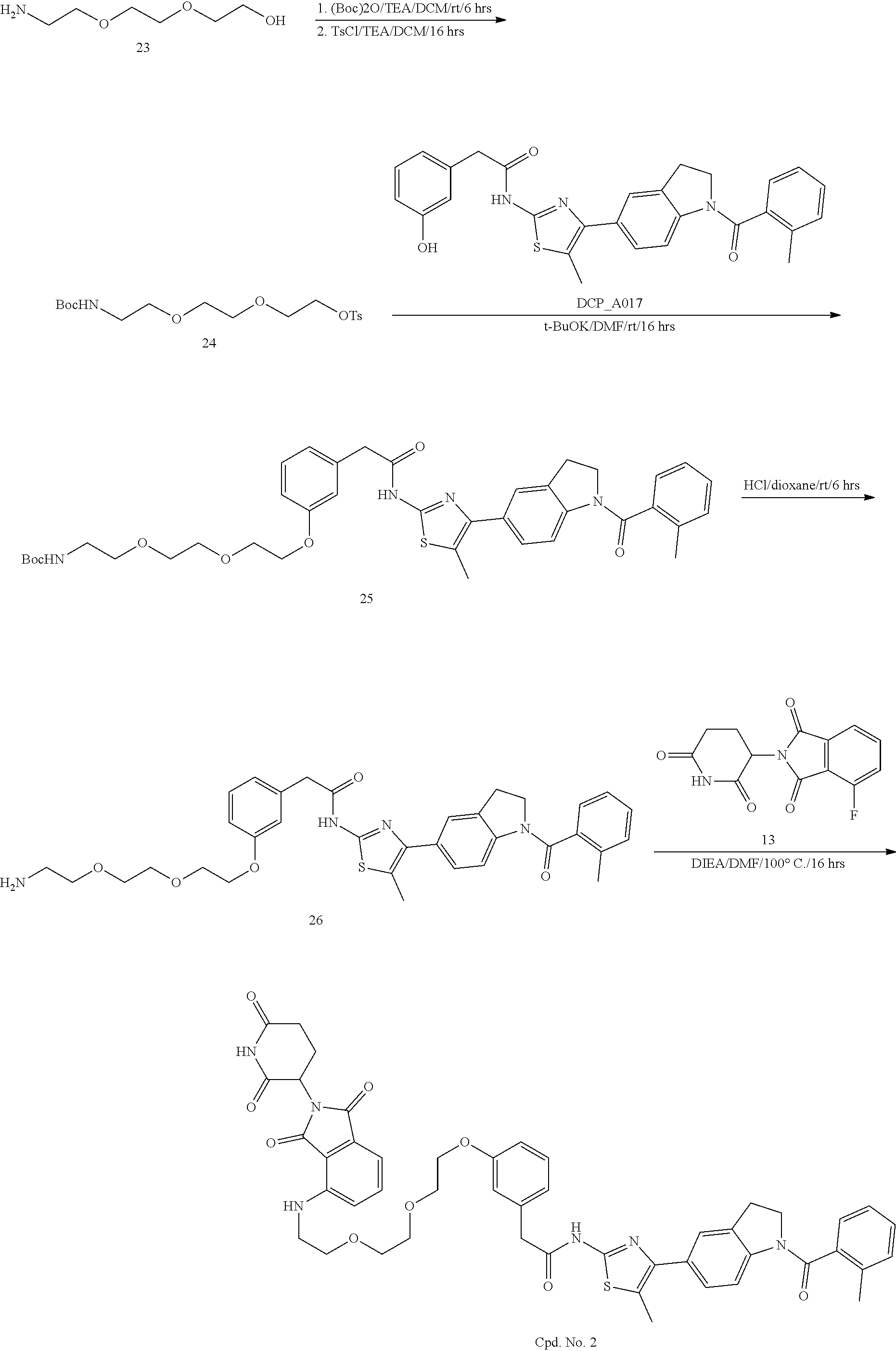

### Step 1. 2,2-dimethyl-4-oxo-3,8,11-trioxa-5-azatridecan-13-yl 4-methylbenzenesulfonate

A solution of 2-(2-(2-aminoethoxy)ethoxy)ethan-1-ol (1.00 g, 6.70 mmol), di-tert-butyl decarbonate (1.75 g, 8.04 mmol) and triethylamine (2.03 g, 20.1 mmol) in dichloromethane (20 mL) was stirred at room temperature for 6 hrs. Tosyl chloride (1.53 g, 8.04 mmol) was added and the mixture was stirred for 16 hrs. The mixture was diluted with water (50 mL), extracted with dichloromethane (100 mL×3), dried over anhydrous sodium sulfate, filtered and concentrated in vacuo. The residue was purified by flash column chromatography (dichloromethane) to give 2,2-dimethyl-4-oxo-3,8,11-trioxa-5-azatridecan-13-yl 4-methylbenzenesulfonate (1.2 g, 44% yield) as yellow oil. MS (ESI) m/z 304.0 [M+H−100]⁺

### Step 2. tert-butyl (2-(2-(2-(3-(2-((5-methyl-4-(1-(2-methylbenzoyl)indolin-5-yl)thiazol-2-yl)amino)-2-oxoethyl)phenoxy)ethoxy)ethoxy)ethyl)carbamate

A solution of 2-(3-hydroxyphenyl)-N-(5-methyl-4-(1-(2-methylbenzoyl)indolin-5-yl)thiazol-2-yl)acetamide (0.50 g, 1.03 mmol), 2,2-dimethyl-4-oxo-3,8,11-trioxa-5-azatridecan-13-yl 4-methylbenzenesulfonate (0.41 g, 1.03 mmol), potassium tert-butoxide (0.65 g, 3.09 mmol) in N,N-dimethylformamide (10 mL) was stirred at room temperature for 16 hrs. The mixture was quenched with water (50 mL), extracted with dichloromethane (50 mL×3), dried over anhydrous sodium sulfate, filtered and concentrated in vacuo. The residue was purified by flash column chromatography (ethyl acetate/petroleum ether=1/10) to give tert-butyl (2-(2-(2-(3-(2-((5-methyl-4-(1-(2-methylbenzoyl)indolin-5-yl)thiazol-2-yl)amino)-2-oxoethyl)phenoxy)ethoxy)ethoxy)ethyl)carbamate (0.40 g, 54% yield) as yellow oil. MS (ESI) m/z 715.5 [M+H]⁺.

### Step 3. 2-(3-(2-(2-(2-aminoethoxy)ethoxy)ethoxy)phenyl)-N-(5-methyl-4-(1-(2-methylbenzoyl)indolin-5-yl)thiazol-2-yl)acetamide

A solution of tert-butyl (2-(2-(2-(3-(2-((5-methyl-4-(1-(2-methylbenzoyl)indolin-5-yl)thiazol-2-yl)amino)-2-oxoethyl)phenoxy)ethoxy)ethoxy)ethyl)carbamate (0.40 g, 0.56 mmol) and 4 M hydrochloric acid/1,4-dioxane (4.0 mL) in 1,4-dioxane (4.0 mL) was stirred at room temperature for 6 hrs. The solvent was removed in vacuo and the residue was dissolved in dichloromethane (100 mL), washed with saturated sodium carbonate aqueous solution, dried over anhydrous sodium sulfate, filtered and concentrated in vacuo. The residue was purified by flash column chromatography (dichloromethane/methanol=1/20) to give 2-(3-(2-(2-(2-aminoethoxy)ethoxy)ethoxy)phenyl)-N-(5-methyl-4-(1-(2-methylbenzoyl)indolin-5-yl)thiazol-2-yl)acetamide (0.28 g, 81% yield) as white solid. MS (ESI) m/z 615.2 [M+H]⁺

### Step 4. 2-(3-(2-(2-(2-((2-(2,6-dioxopiperidin-3-yl)-1,3-dioxoisoindolin-4-yl)amino)ethoxy)ethoxy)ethoxy)phenyl)-N-(5-methyl-4-(1-(2-methylbenzoyl)indolin-5-yl)thiazol-2-yl)acetamide

A solution of 2-(3-(2-(2-(2-aminoethoxy)ethoxy)ethoxy)phenyl)-N-(5-methyl-4-(1-(2-methylbenzoyl)indolin-5-yl)thiazol-2-yl)acetamide (0.28 g, 0.45 mmol), 2-(2,6-dioxopiperidin-3-yl)-4-fluoroisoindoline-1,3-dione (0.15 g, 0.54 mmol) and N, N-Diisopropylethylamine (0.17 g, 1.35 mmol) in N,N-dimethylformamide (5.0 mL) was stirred at 100° C. for 16 hrs. The reaction mixture was purified by Prep-HPLC (Column: Welch Xtimate 21.2×250 mm C18, 10 μm, Mobile Phase: A: water (10 mmol/L ammonium bicarbonate) B: acetonitrile; B %: 30%-70% in 15.0 min) to give 2-(3-(2-(2-(2-((2-(2,6-dioxopiperidin-3-yl)-1,3-dioxoisoindolin-4-yl)amino)ethoxy)ethoxy)ethoxy)phenyl)-N-(5-methyl-4-(1-(2-methylbenzoyl)indolin-5-yl)thiazol-2-yl)acetamide (52.0 mg, 13.1% yield) as yellow solid. ¹H NMR (400 MHz, DMSO-d₆): δ12.34 & 12.27 (s, 1H)*, 11.10 (s, 1H), 8.24 & 5.54* (d, J=8.8 Hz, 1H), 7.57-7.52 (m, 3H), 7.37-7.34 (m, 7.22 (t, J=7.8 Hz, 1H), 7.12 (d, J=8.8 Hz, 1H), 7.02 (d, J=6.8 Hz, 1H), 6.91-6.88 (m, 2H), 6.82 (d, J=8.0 Hz, 1H), 6.61 (t, J=5.4 Hz, 1H), 5.05 (dd, J=12.6 & 5.4 Hz, 1H), 4.21 & 3.70 (m, 2H)*, 4.05 (m, 2H), 3.73-3.70 (m, 3.60 (m, 6H), 3.47-3.45 (m, 2H), 3.13 (t, J=7.4 Hz, 2H), 2.91-2.82 (m, 1H), 2.57-2.54 (m, 2H), 2.45 & 2.34 (s, 3H)*, 2.29 & 2.18 (s, 3H)*, 2.02-1.99 (m, 1H). MS (ESI) m/z 871.3 [M+H]⁺. *Multiple signals arising from conformational isomers.

### Example 7

### Synthesis of 2-(3-(2-(2-(2-((2-(2,6-dioxopiperidin-3-yl)-1,3-dioxoisoindolin-5-yl)amino)ethoxy)ethoxy)ethoxy)phenyl)-N-(5-methyl-4-(1-(2-methylbenzoyl)indolin-5-yl)thiazol-2-yl)acetamide (Cpd. No. 21)

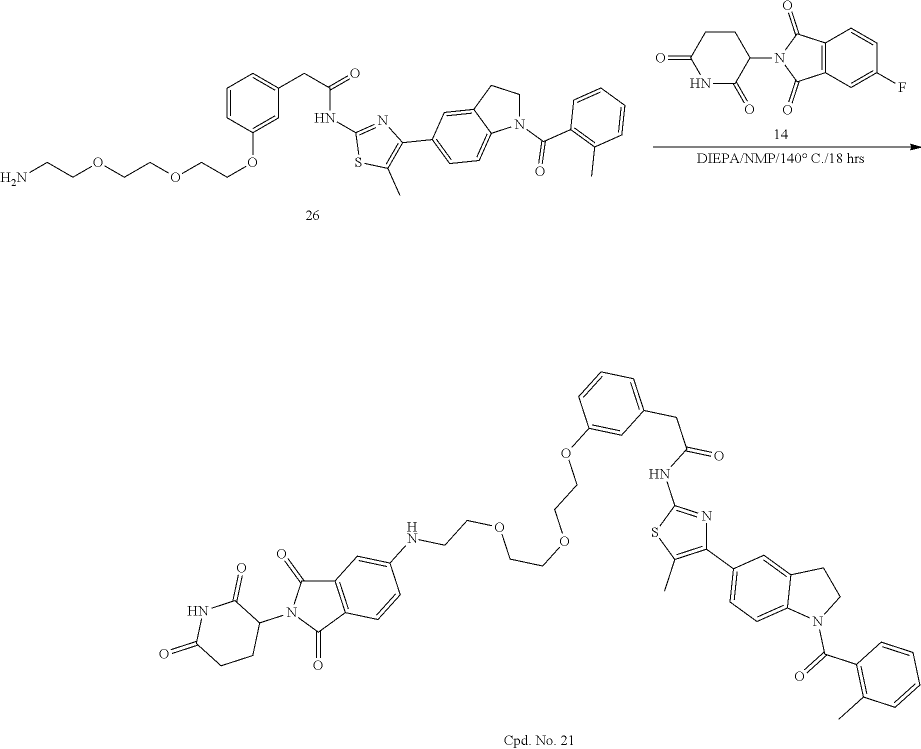 [SMILES: Cc1ccccc1C(=O)N1CCc2cc(-c3nc(NC(=O)Cc4cccc(OCCOCCOCCN)c4)sc3C)ccc21 ; O=C1CCC(N2C(=O)c3ccc(F)cc3C2=O)C(=O)N1 ; Cc1ccccc1C(=O)N1CCc2cc(-c3nc(NC(=O)Cc4cccc(OCCOCCOCCNc5ccc6c(c5)C(=O)N(C5CCC(=O)NC5=O)C6=O)c4)sc3C)ccc21]

### Step 1. 2-(3-(2-(2-(2-((2-(2,6-dioxopiperidin-3-yl)-1,3-dioxoisoindolin-5-yl)amino)ethoxy)ethoxy)ethoxy)phenyl)-N-(5-methyl-4-(1-(2-methylbenzoyl)indolin-5-yl)thiazol-2-yl)acetamide

A solution of 2-(3-(2-(2-(2-aminoethoxy)ethoxy)ethoxy)phenyl)-N-(5-methyl-4-(1-(2-methylbenzoyl)indolin-5-yl)thiazol-2-yl)acetamide (0.070 g, 0.11 mmol), 2-(2,6-dioxopiperidin-3-yl)-5-fluoroisoindoline-1,3-dione (0.031 g, 0.11 mmol) and N, N-Diisopropylethylaamine (0.044 g, 0.34 mmol) in 1-methylpyrrolidin-2-one (3.0 mL) was stirred at 140° C. for 18 hrs. The reaction mixture was purified by Prep-HPLC (Column: Welch Xtimate 21.2×250 mm C18, 10 μm, Mobile Phase: A: water (10 mmol/L ammonium bicarbonate) B: acetonitrile; B %: 30%-70% in 15.0 min) to give 2-(3-(2-(2-(2-((2-(2,6-dioxopiperidin-3-yl)-1,3-dioxoisoindolin-5-yl)amino)ethoxy)ethoxy)ethoxy)phenyl)-N-(5-methyl-4-(1-(2-methylbenzoyl)indolin-5-yl)thiazol-2-yl)acetamide (20 mg, 21% yield) as a yellow solid. ¹H NMR (400 MHz, DMSO-d₆): δ12.34 & 12.27 (s, 1H)*, 11.07 (s, 1H), 8.24 & 5.54* (d, J=8.8 Hz, 1H), 7.55-7.52 (m, 3H), 7.37-7.30 (m, 5H), 7.23 (t, J=6.2 Hz, 1H), 7.0 (s, 1H), 6.92-6.87 (m, 3H), 6.82 (d, J=6.4 Hz, 1H), 5.03 (dd, J=10.0 & 4.4 Hz, 1H), 4.21 & 3.70 (m, 2H)*, 4.06 (m, 2H), 3.73-3.70 (m, 3.60-3.59 (m, 6H), 3.35 (t, J=4.2 Hz, 2H), 3.13 (t, J=6.6 Hz, 2H), 2.89-2.86 (m, 1H), 2.58-2.54 (m, 2H), 2.45 & 2.34 (s, 3H)*, 2.29 & 2.17 (s, 3H)*, 1.99-1.96 (m, 1H). MS (ESI) m/z 871.4 [M+H]⁺ *Multiple signals arising from conformational isomers.

### Example 8

### Synthesis of 2-(3-(2-(2-(2-((2-(2,6-dioxopiperidin-3-yl)-1,3-dioxoisoindolin-4-yl)oxy)ethoxy)ethoxy)ethoxy)phenyl)-N-(5-methyl-4-(1-(2-methylbenzoyl)indolin-5-yl)thiazol-2-yl)acetamide (Cpd. No. 7)

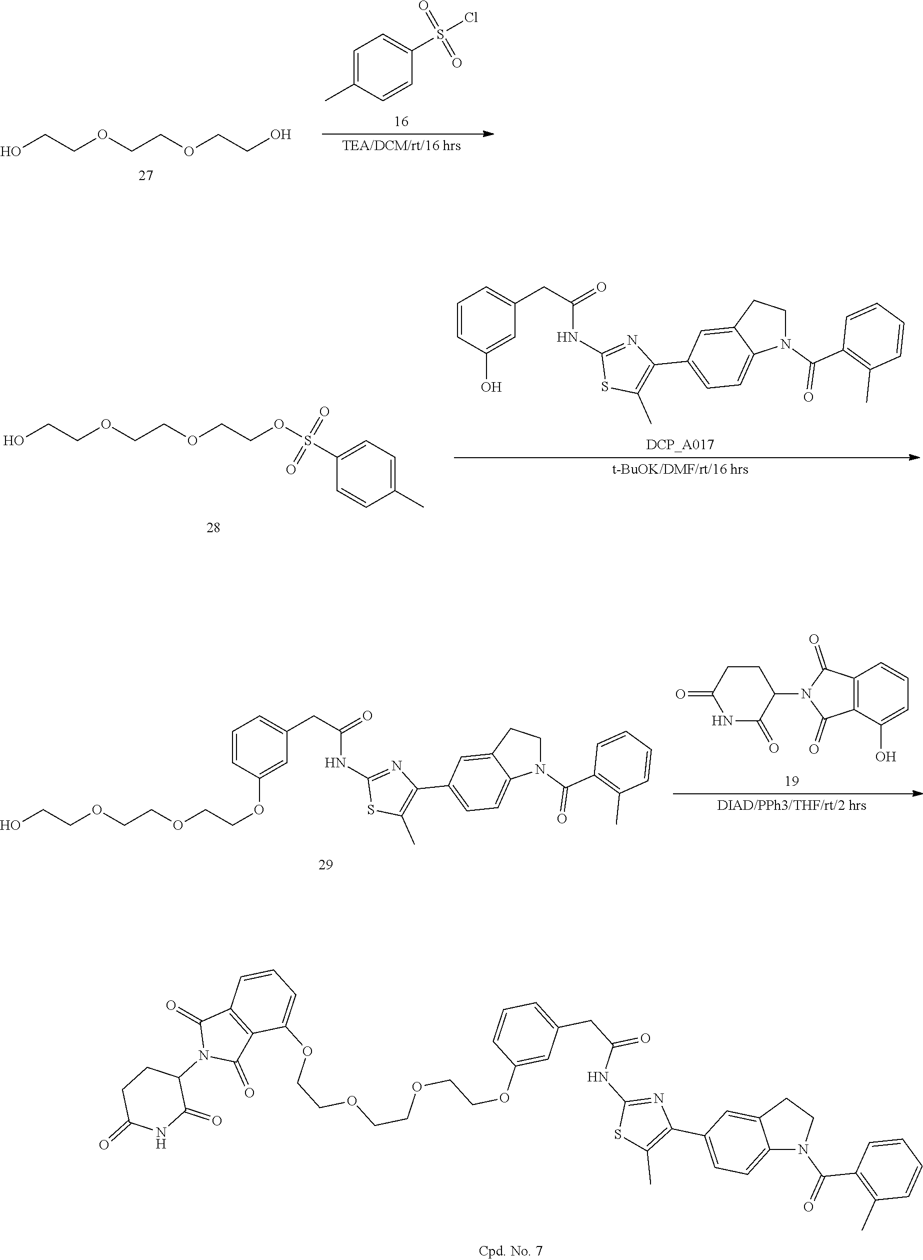

### Step 1. 2-(2-(2-hydroxyethoxy)ethoxy)ethyl 4-methylbenzenesulfonate

A solution of 2,2′-(ethane-1,2-diylbis(oxy))diethanol (5.00 g, 33.3 mmol), 4-methylbenzene-1-sulfonyl chloride (3.17 g, 16.7 mmol), triethylamine (6.74 g, 66.6 mmol) in dichloromethane (100 mL) was stirred at room temperature for 16 hrs. The solvent was removed in vacuo and the residue was purified by flash column chromatography (ethyl acetate/petroleum ether=1:1) to give 2-(2-(2-hydroxyethoxy)ethoxy)ethyl 4-methylbenzenesulfonate (2.0 g, 39% yield) as a yellow oil. MS (ESI) m/z 305.3 [M+H]⁺.

### Step 2. 2-(3-(2-(2-(2-hydroxyethoxy)ethoxy)ethoxy)phenyl)-N-(5-methyl-4-(1-(2-methylbenzoyl)indolin-5-yl)thiazol-2-yl)acetamide

A solution of 2-(3-hydroxyphenyl)-N-(5-methyl-4-(1-(2-methylbenzoyl)indolin-5-yl)thiazol-2-yl)acetamide (1.00 g, 2.07 mmol), 2-(2-(2-hydroxyethoxy)ethoxy)ethyl 4-methylbenzenesulfonate (0.69 g, 2.27 mmol), potassium tert-butoxide (0.69 g, 6.2 mmol) in N,N-dimethylformamide (30 mL) was stirred at room temperature for 16 hrs. The mixture was diluted with water (20 mL), extracted with dichloromethane (50 mL), washed with brine (10 mL×3), dried over anhydrous sodium sulfate, filtered and concentrated in vacuo. The residue was purified by flash column chromatography (dichloromethane/methanol=5/100) to give 2-(3-(2-(2-(2-hydroxyethoxy)ethoxy)ethoxy)phenyl)-N-(5-methyl-4-(1-(2-methylbenzoyl)indolin-5-yl)thiazol-2-yl)acetamide (0.70 g, 55.1% yield) as a yellow solid. MS (ESI) m/z 616.4 [M+]⁺.

### Step 3. 2-(3-(2-(2-(2-((2-(2,6-dioxopiperidin-3-yl)-1,3-dioxoisoindolin-4-yl)oxy)ethoxy)ethoxy)ethoxy)phenyl)-N-(5-methyl-4-(1-(2-methylbenzoyl)indolin-5-yl)thiazol-2-yl)acetamide

To a solution of 2-(3-(2-(2-(2-hydroxyethoxy)ethoxy)ethoxy)phenyl)-N-(5-methyl-4-(1-(2-methylbenzoyl)indolin-5-yl)thiazol-2-yl)acetamide (0.10 g, 0.16 mmol), 2-(2,6-dioxopiperidin-3-yl)-4-hydroxyisoindoline-1,3-dione (0.049 g, 0.18 mmol) and triphenylphosphine (0.089 g, 0.34 mmol) in tetrahydrofuran (2.0 mL), was added diisopropyl azodicarboxylate (0.049 g, 0.24 mmol) at room temperature. The mixture was stirred at room temperature for 2 hrs. The mixture was diluted with water (10 mL), extracted with dichloromethane (50 mL), dried over anhydrous sodium sulfate, filtered and concentrated in vacuo. The residue was purified by Prep-HPLC (Column: Welch Xtimate 21.2×250 mm C18, 10 μm, Mobile Phase: A: water (10 mmol/L ammonium bicarbonate) B: acetonitrile; B %: 30%-70% in 15.0 min) to give 2-(3-(2-(2-(2-((2-(2,6-dioxopiperidin-3-yl)-1,3-dioxoisoindolin-4-yloxy)ethoxy)ethoxy)ethoxy)phenyl)-N-(5-methyl-4-(1-(2-methylbenzoyl)indolin-5-yl)thiazol-2-yl)acetamide (52 mg, 37% yield) as white solid. ¹H NMR (400 MHz, DMSO-d₆): δ12.33 & 12.22 (s, 1H)*, 11.10 (s, 1H), 8.23 & 5.56* (d, J=8.4 Hz, 1H), 7.77 (t, J=7.8 Hz, 1H), 7.52-7.59 (m, 2H), 7.52 &7.03* (m, 1H), 7.43 (d, J=7.2 Hz, 1H), 7.37-7.35 (m, 7.22 (t, J=7.8 Hz, 1H), 6.91-6.88 (m, 2H), 6.82 (d, J=8.0 Hz, 1H), 5.07 (dd, J=12.8 & 5.6 Hz, 1H), 4.33 (t, J=4.4 Hz, 2H), 4.21 & 3.74 (m, 2H)*, 4.06 (t, J=4.2 Hz, 2H), 3.81 (t, J=4.2 Hz, 2H), 3.82-3.66 (m, 6H), 3.62-3.60 (m, 2H), 3.13 (t, J=7.8 Hz, 2H), 2.88-2.82 (m, 1H), 2.59-2.55 (m, 2H), 2.48 & 2.33 (s, 3H)*, 2.29 & 2.17 (s, 3H)*, 2.03-1.98 (m, 1-1). MS (ESI) m/z 872.3 [M+H]⁺. *Multiple signals arising from conformational isomers.

### Example 9

### Synthesis of 2-(3-(2-(2-(2-((2-(2,6-dioxopiperidin-3-yl)-1,3-dioxoisoindolin-5-yl)oxy)ethoxy)ethoxy)ethoxy)phenyl)-N-(5-methyl-4-(1-(2-methylbenzoyl)indolin-5-yl)thiazol-2-yl)acetamide (Cpd. No. 19)

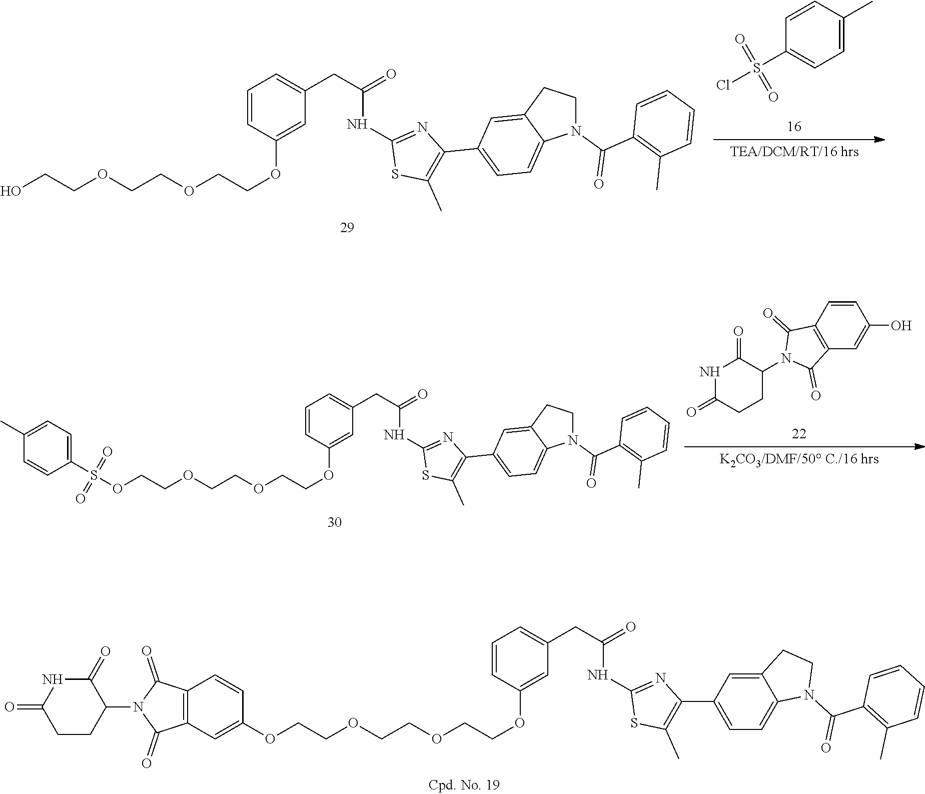

### Step 1. 2-(2-(2-(3-(2-((5-methyl-4-(1-(2-methylbenzoyl)indolin-5-yl)thiazol-2-yl)amino)-2-oxoethyl)phenoxy)ethoxy)ethoxy)ethyl 4-methylbenzenesulfonate

A solution of 2-(3-(2-(2-(2-hydroxyethoxy)ethoxy)ethoxy)phenyl)-N-(5-methyl-4-(1-(2-methylbenzoyl)indolin-5-yl)thiazol-2-yl)acetamide (140 mg, 0.22 mmol), tosyl chloride (63.0 mg, 0.33 mmol) and triethylamine (69 mg, 0.66 mmol) in dichloromethane (10 mL) was stirred at room temperature for 16 hrs. The mixture was diluted with water (10 mL), extracted with dichloromethane (30 mL), dried over anhydrous sodium sulfate, filtered and concentrated in vacuo. The residue was purified by flash column chromatography (dichloromethane) to give 2-(2-(2-(3-(2-((5-methyl-4-(1-(2-methylbenzoyl)indolin-5-yl)thiazol-2-yl)amino)-2-oxoethyl)phenoxy)ethoxy)ethoxy)ethyl 4-methylbenzenesulfonate (90 mg, 51.4% yield) as a yellow oil. MS (ESI) m/z 770.1 [M+H]⁺.

### Step 2. 2-(3-(2-(2-(2-((2-(2,6-dioxopiperidin-3-yl)-1,3-dioxoisoindolin-5-yl)oxy)ethoxy)ethoxy)ethoxy)phenyl)-N-(5-methyl-4-(1-(2-methylbenzoyl)indolin-5-yl)thiazol-2-yl)acetamide

A solution of 2-(2-(2-(3-(2-((5-methyl-4-(1-(2-methylbenzoyl)indolin-5-yl)thiazol-2-yl)amino)-2-oxoethyl)phenoxy)ethoxy)ethoxy)ethyl 4-methylbenzenesulfonate (90.0 mg, 0.12 mmol), 2-(2,6-dioxopiperidin-3-yl)-5-hydroxyisoindoline-1,3-dione (55.8 mg, 0.20 mmol) and potassium carbonate (70.3 mg, 0.51 mmol) in N,N-dimethylformamide (2.0 mL) was stirred at 50° C. for 16 hrs. The reaction mixture was purified by Prep-HPLC (Column: Welch Xtimate 21.2×250 mm C18, 10 μm, Mobile Phase: A: water (10 mmol/L ammonium bicarbonate) B: acetonitrile; B %: 30%-70% in 15.0 min) to give 2-(3-(2-(2-(2-((2-(2,6-dioxopiperidin-3-yl)-1,3-dioxoisoindolin-5-yl)oxy)ethoxy)ethoxy)ethoxy)phenyl)-N-(5-methyl-4-(1-(2-methylbenzoyl)indolin-5-yl)thiazol-2-yl)acetamide (4.5 mg, 4.4% yield) as a white solid. ¹H NMR (400 MHz, DMSO-d₆): δ 8.23 & 5.45* (d, J=8.0 Hz, 1H), 7.81 (d, J=8.4 Hz, 1H), 7.52 (brs, 1H), 7.52 & 7.01*(brs, 1H), 7.44 (d, J=1.6 Hz, 1H), 7.37-7.30 (m, 5H), 7.23 (t, J=7.8 Hz, 1H), 6.92-6.89 (m, 2H), 6.83 (d, J=8.4 Hz, 1H), 5.11 (dd, J=12.8 & 5.6 Hz, 1H), 4.29 (t, J=4.4 Hz, 2H), 4.21 & 3.70 (m, 2H)*, 4.07 (m, 2H), 3.80-3.70 (m, 6H), 3.62 (s, 4H), 3.12 (t, J=8.0 Hz, 2H), 2.88-2.85 (m, 1H), 2.60-2.55 (m, 2H), 2.45 & 2.33 (s, 3H)*, 2.29 & 2.18 (s, 3H)*, 2.05-2.00 (m, 1H). MS (ESI) m/z 872.3 [M+H]⁺. (Note: 2 acidic protons that are typically around 12.3 ppm and 11.1 ppm are not visible). *Multiple signals arising from conformational isomers.

### Example 10

### Synthesis of 2-(3-(2-(2-(2-(2-((2-(2,6-dioxopiperidin-3-yl)-1,3-dioxoisoindolin-4-yl)amino)ethoxy)ethoxy)ethoxy)ethoxy)phenyl)-N-(5-methyl-4-(1-(2-methylbenzoyl)indolin-5-yl)thiazol-2-yl)acetamide (Cpd. No. 3)

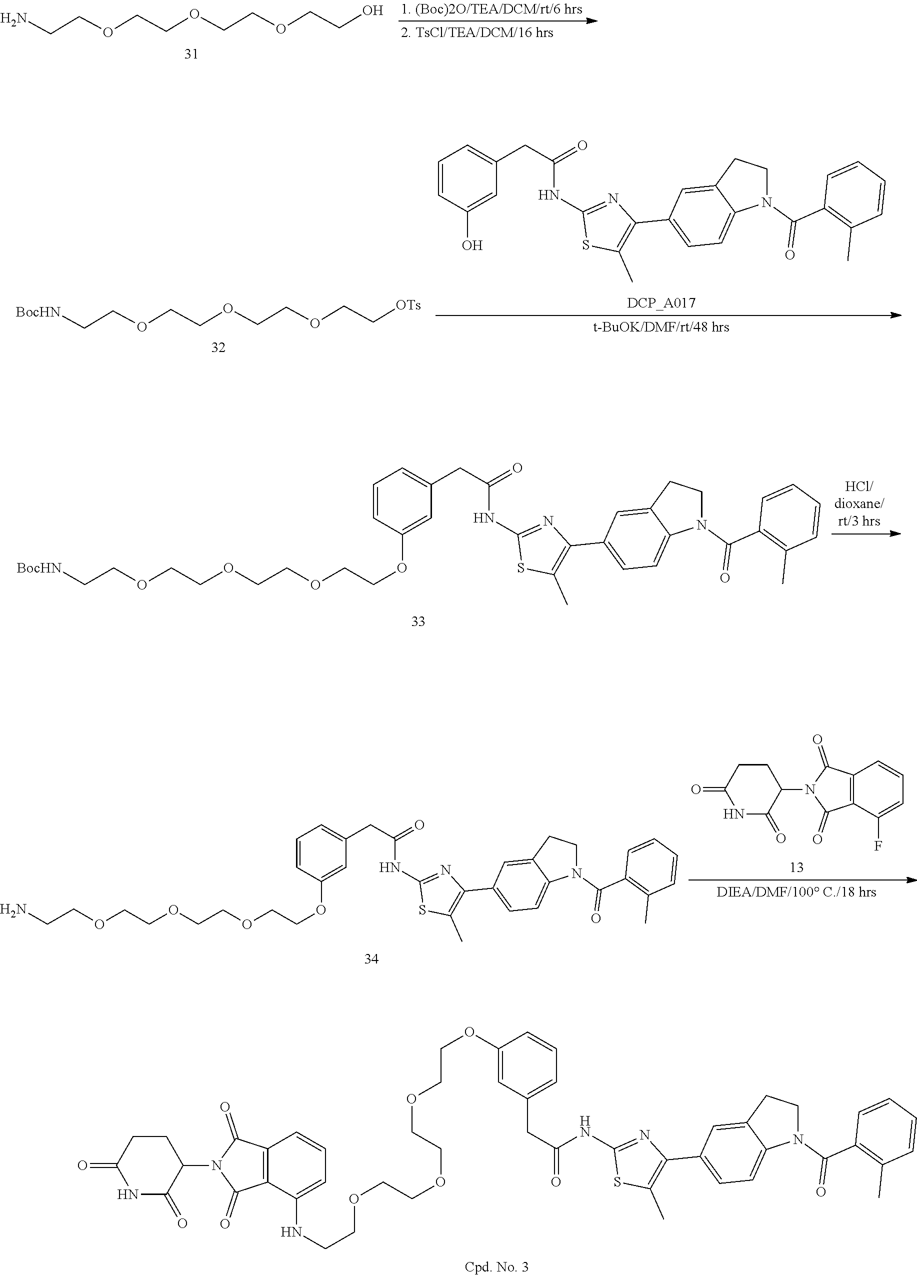

### Step 1. 2,2-dimethyl-4-oxo-3,8,11,14-tetraoxa-5-azahexadecan-16-yl 4-methylbenzenesulfonate

A solution of 2-(2-(2-(2-aminoethoxy)ethoxy)ethoxy)ethan-1-ol (1.0 g, 5.18 mmol), di-tert-butyl decarbonate (1.13 g, 5.18 mmol) and triethylanine (1.56 g, 15.5 mmol) in dichloromethane (10 mL) was stirred at room temperature for 6 hrs. Tosyl chloride (1.00 g, 5.18 mmol) was added and the mixture was stirred at room temperature 16 hrs. The mixture was diluted with water (50 mL), extracted with dichloromethane (100 mL), dried over anhydrous sodium sulfate, filtered and concentrated in vacuo. The residue was purified by flash column chromotography (dichloromethane) to give 2,2-dimethyl-4-oxo-3,8,11,14-tetraoxa-5-azahexadecan-16-yl 4-methylbenzenesulfonate (0.45 g, 19% yield) as a yellow oil. MS (ESI) m/z 348.2 [M+H−100]⁺.

### Step 2. tert-butyl (2-(2-(2-(2-(3-(2-((5-methyl-4-(1-(2-methylbenzoyl)indolin-5-yl)thiazol-2-yl)amino)-2-oxoethyl)phenoxy)ethoxy)ethoxy)ethoxy)ethyl)carbamate

A solution of 2-(3-hydroxyphenyl)-N-(5-methyl-4-(1-(2-methylbenzoyl)indolin-5-yl)thiazol-2-yl)acetamide (0.40 g, 0.83 mmol), 2,2-dimethyl-4-oxo-3,8,11,14-tetraoxa-5-azahexadecan-16-yl 4-methylbenzenesulfonate (0.45 g, 1.0 mmol), potassium tert-butoxide (0.37 g, 3.3 mmol) in N,N-dimethylformamide (5.0 mL) was stirred at room temperature for 48 hrs. The mixture was quenched with water (50 mL), extracted with dichloromethane (50 mL×2), dried over anhydrous sodium sulfate, filtered and concentrated in vacuo. The residue was purified by flash column chromatography (ethyl acetate/petroleum ether=3/10) to give crude tert-butyl (2-(2-(2-(2-(3-(2-((5-methyl-4-(1-(2-methylbenzoyl)indolin-5-yl)thiazol-2-yl)amino)-2-oxoethyl)phenoxy)ethoxy)ethoxy)ethoxy)ethyl)carbamate (0.15 g, 23% yield) as a yellow oil. MS (ESI) m/z 759.3 [M+1H]⁺.

### Step 3. 2-(3-(2-(2-(2-(2-aminoethoxy)ethoxy)ethoxy)ethoxy)phenyl)-N-(5-methyl-4-(1-(2-methylbenzoyl)indolin-5-yl)thiazol-2-yl)acetamide

A solution of tert-butyl (2-(2-(2-(2-(3-(2-((5-methyl-4-(1-(2-methylbenzoyl)indolin-5-yl)thiazol-2-yl)amino)-2-oxoethyl)phenoxy)ethoxy)ethoxy)ethoxy)ethyl)carbamate (0.15 g, 0.20 mmol) and hydrochloric acid (4 M in 1,4-dioxane, 5.0 mL) in 1,4-dioxane (10.0 mL) was stirred at room temperature for 3 hrs. The solvent was removed and the residue was dissolved in dichloromethane (100 mL), washed with saturated sodium carbonate solution (20 mL×2), dried over anhydrous sodium, filtered and concentrated in vacuo. The residue was purified by flash column chromatography (dichloromethane/methanol=1/20) to give 2-(3-(2-(2-(2-(2-aminoethoxy)ethoxy)ethoxy)ethoxy)phenyl)-N-(5-methyl-4-(1-(2-methylbenzoyl)indolin-5-yl)thiazol-2-yl)acetamide (100 mg, 76% yield) as a white solid. MS (ESI) m/z 659.5 [M+H]⁺.

### Step 4. 2-(3-(2-(2-(2-(2-((2-(2,6-dioxopiperidin-3-yl)-1,3-dioxoisoindolin-4-yl)amino)ethoxy)ethoxy)ethoxy)ethoxy)phenyl)-N-(5-methyl-4-(1-(2-methylbenzoyl)indolin-5-yl)thiazol-2-yl)acetamide

A solution of 2-(3-(2-(2-(2-(2-aminoethoxy)ethoxy)ethoxy)ethoxy)phenyl)-N-(5-methyl-4-(1-(2-methylbenzoyl)indolin-5-yl)thiazol-2-yl)acetamide (0.090 g, 0.14 mmol), 2-(2,6-dioxopiperidin-3-yl)-4-fluoroisoindoline-1,3-dione (0.048 g, 0.17 mmol) and N, A diisopropylethylamine (0.060 g, 0.45 mmol) in N,N-dimethylformamide (2.0 mL) was stirred at 100° C. for 18 hrs. The reaction mixture was purified by Prep-HPLC (Column: Welch Xtimate 21.2×250 mm C18, 10 μm, Mobile Phase: A: water (10 mmol/L ammonium bicarbonate) B: acetonitrile; B %: 30%-70% in 15.0 min) to give 2-(3-(2-(2-(2-(2-((2-(2,6-dioxopiperidin-3-yl)-1,3-dioxoisoindolin-4-yl)amino)ethoxy)ethoxy)ethoxy)ethoxy)phenyl)-N-(5-methyl-4-(1-(2-methylbenzoyl)indolin-5-yl)thiazol-2-yl)acetamide (30.1 mg, 24% yield) as a yellow solid. ¹H NMR (400 MHz, DMSO-d₆): δ12.34 & 12.27 (s, 1H)*, 11.11 (s, 1H), 8.24 & 5.54* (d, J=8.8 Hz, 1H), 7.58-7.53 (m, 3H), 7.38-7.36 (m, 4H), 7.23 (t, J=7.8 Hz, 1 H), 7.12 (d, J=8.8 Hz, 1H), 7.03 (d, J=6.8 Hz, 1H), 6.92-6.90 (m, 2H), 6.83 (d, J=7.6, 1H), 6.60 (t, J=5.8 Hz, 1H), 5.05 (dd, J=12.8 & 5.2 Hz, 1H), 4.21 & 3.70 (m, 2H)*, 4.05 (m, 2H), 3.73-3.71 (m, 3.62-3.55 (m, 10H), 3.47-3.44 (m, 2H), 3.13 (t, J=8.0 Hz, 2H), 2.91-2.82 (m, 1H), 2.60-2.54 (m, 2H), 2.45 & 2.34 (s, 3H)*, 2.30 & 2.18 (s, 3H)*, 2.03-1.24 (m, 1H). MS (ESI) m/z 915.3 [M+H]⁺. *Multiple signals arising from conformational isomers.

### Example 11

### Synthesis of 2-(3-(2-(2-(2-(2-((2-(2,6-dioxopiperidin-3-yl)-1,3-dioxoisoindolin-4-yl)oxy)ethoxy)ethoxy)ethoxy)ethoxy)phenyl)-N-(5-methyl-4-(1-(2-methylbenzoyl)indolin-5-yl)thiazol-2-yl)acetamide (Cpd. No. 8)

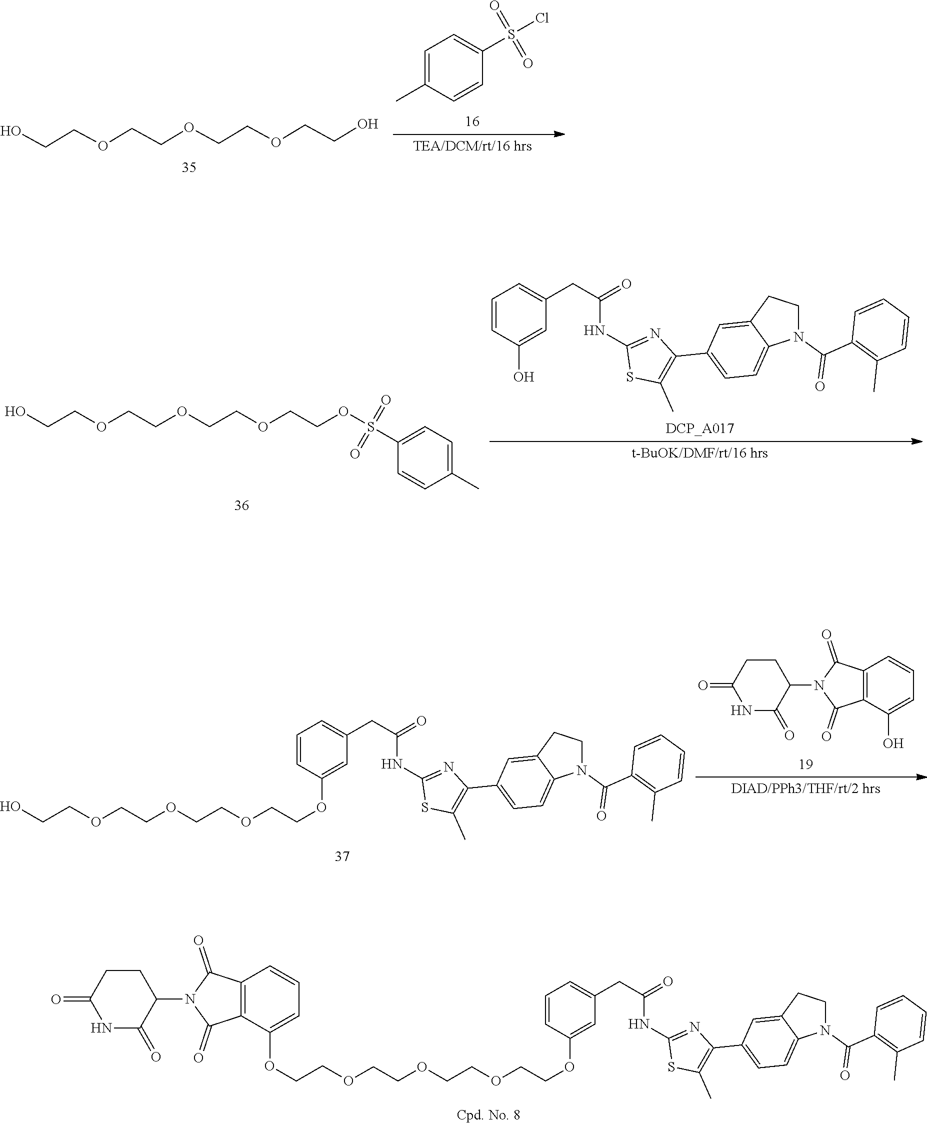

### Step 1. 2-(2-(2-(2-hydroxyethoxy)ethoxy)ethoxy)ethyl 4-methylbenzenesulfonate

A solution of 2,2′-((oxybis(ethane-2,1-diyl))bis(oxy))bis(ethan-1-ol) (5.0 g, 25.7 mmol), 4-methylbenzenesulfonyl chloride (2.4 g, 12.6 mmol), triethylamine (7.8 g, 77.2 mmol) in dichloromethane (100 mL) was stirred at room temperature for 16 hrs. The mixture was quenched with water (30 mL), extracted with ethyl acetate (50 mL×2), dried over anhydrous sodium sulfate, filtered and concentrated in vacuo. The residue was purified by flash column chromatography (ethyl acetate/petroleum ether=1:1) to give 2-(2-(2-(2-hydroxyethoxy)ethoxy)ethoxy)ethyl 4-methylbenzenesulfonate (2.0 g, 45% yield) as a yellow oil. MS (ESI) m/z 348.4 [M+H]⁺.

### Step 2. 2-(3-(2-(2-(2-(2-hydroxyethoxy)ethoxy)ethoxy)ethoxy)phenyl)-N-(5-methyl-4-(1-(2-methylbenzoyl)indolin-5-yl)thiazol-2-yl)acetamide

A mixture of 2-(2-(2-(2-hydroxyethoxy)ethoxy)ethoxy)ethyl 4-methylbenzenesulfonate (0.81 g, 2.32 mmol), 2-(3-hydroxyphenyl)-N-(5-methyl-4-(1-(2-methylbenzoyl)indolin-5-yl)thiazol-2-yl)acetamide (1.00 g, 2.07 mmol), potassium t-butoxide (0.49 g, 4.34 mmol) in dry NA-dimethylformamide (20 mL) was stirred at room temperature for 16 hrs. The reaction mixture was quenched with water (20 mL), extracted with ethyl acetate (30 mL×2), dried over sodium sulfate, filtered and concentrated in vacuo. The residue was purified by flash column chromatography (ethyl acetate/petroleum ether=1/1) to give 2-(3-(2-(2-(2-(2-hydroxyethoxy)ethoxy)ethoxy) ethoxy)phenyl)-N-(5-methyl-4-(1-(2-methylbenzoyl)indolin-5-yl)thiazol-2-yl)acetamide (0.66 g, 48.4% yield) as a white solid. MS (ESI) m/z 660.3 [M+H]⁺.

### Step 3. 2-(3-(2-(2-(2-(2-((2-(2,6-dioxopiperidin-3-yl)-1,3-dioxoisoindolin-4-yl)oxy)ethoxy)ethoxy)ethoxy)ethoxy)phenyl)-N-(5-methyl-4-(1-(2-methylbenzoyl)indolin-5-yl)thiazol-2-yl)acetamide

To a solution of 2-(3-(2-(2-(2-(2-hydroxyethoxy)ethoxy)ethoxy)ethoxy)phenyl)-N-(5-methyl-4-(1-(2-methylbenzoyl)indolin-5-yl)thiazol-2-yl)acetamide (0.13 g, 0.20 mmol) and 2-(2,6-dioxopiperidin-3-yl)-4-hydroxyisoindoline-1,3-dione (0.054 g, 0.20 mmol), triphenylphosphine (0.052 mg, 0.20 mmol) in tetrahydrofuran (3.0 mL), was added diisopropyl azodiformate (0.048 g, 0.24 mmol) at 0° C. slowly. The reaction was stirred at room temperature for 2 hrs. The reaction mixture was purified by Prep-HPLC (Column: Welch Xtimate 21.2×250 mm C18, 10 μm, Mobile Phase: A: water (10 mmol/L ammonium bicarbonate) B: acetonitrile; B %: 30%-70% in 15.0 min) to give 2-(3-(2-(2-(2-(2-((2-(2,6-dioxopiperidin-3-yl)-1,3-dioxoisoindolin-4-yl)oxy)ethoxy)ethoxy) ethoxy)ethoxy)phenyl)-N-(5-methyl-4-(1-(2-methylbenzoyl)indolin-5-yl)thiazol-2-yl) acetamide (26.7 mg, 14.8% yield) as a white solid. ¹H NMR (400 MHz, DMSO-d₆): δ12.35 & 12.22 (s, 1H)*, 11.12 (s, 1H), 8.24 & 5.56* (d, J=8.8 Hz, 1H), 7.79 (t, J=7.8 Hz, 1H), 7.53-7.49 (m, 3H), 7.44 (d, J=7.2 Hz, 1H), 7.38-7.29 (m, 7.23 (t, J=7.6 Hz, 1H), 6.92-6.89 (m, 2H), 6.83 (d, J=7.2 Hz, 1H), 5.08 (dd, J=13.4 & 5.4 Hz, 1H), 4.32 (t, J=4.4 Hz, 2H), 4.21 & 3.74 (m, 2H)*, 4.06 (t, J=4.0 Hz, 2H), 3.79 (t, J=4.2 Hz, 2H), 3.74-3.70 (m, 3.65-3.63 (m, 2H), 3.57-3.49 (m, 6H), 3.13 (t, J=8.4 Hz, 2H), 2.89-2.84 (m, 1H), 2.59-2.55 (m, 2H), 2.45 & 2.33 (s, 3H)*, 2.29 & 2.17 (s, 3H)*, 2.03-1.98 (m, 1H). MS (ESI) m/z 916.2 [M+H]⁺. *Multiple signals arising from conformational isomers.

### Example 12

### Synthesis of 2-(3-(2-(2-(2-(2-((2-(2,6-dioxopiperidin-3-yl)-1,3-dioxoisoindolin-5-yl)oxy)ethoxy)ethoxy)ethoxy)ethoxy)phenyl)-N-(5-methyl-4-(1-(2-methylbenzoyl)indolin-5-yl)thiazol-2-yl)acetamide (Cpd. No. 13)

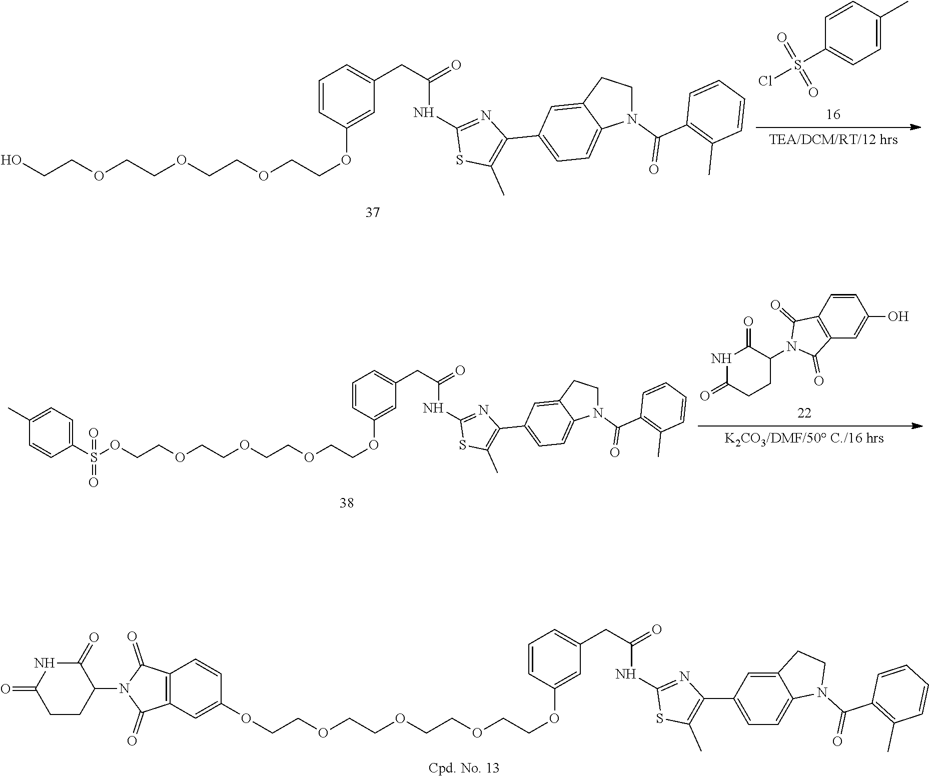

### Step 1. 2-(2-(2-(2-(3-(2-((5-methyl-4-(1-(2-methylbenzoyl)indolin-5-yl)thiazol-2-yl)amino)-2-oxoethyl)phenoxy)ethoxy)ethoxy)ethoxy)ethyl 4-methylbenzenesulfonate

A solution of 2-(3-(2-(2-(2-(2-hydroxyethoxy)ethoxy)ethoxy)ethoxy)phenyl)-N-(5-methyl-4-(1-(2-methylbenzoyl)indolin-5-yl)thiazol-2-yl)acetamide (0.25 g, 0.38 mmol), tosyl chloride (0.073 g, 0.38 mmol) and triethylamine (0.091 g, 0.9 mmol) in dichloromethane (10 mL) was stirred at room temperature for 12 hrs. The mixture was diluted with water (10 mL), extracted with dichloromethane (30 mL), dried over anhydrous sodium sulfate, filtered and concentrated in vacuo. The residue was purified by flash column chromatography (dichloromethane) to give 2-(2-(2-(2-(3-(2-((5-methyl-4-(1-(2-methylbenzoyl)indolin-5-yl)thiazol-2-yl)amino)-2-oxoethyl)phenoxy)ethoxy)ethoxy)ethoxy)ethyl 4-methylbenzenesulfonate (0.12 g, 39% yield) as a yellow oil. MS (ESI) m/z 814.4 [M+H]⁺.

### Step 2. 2-(3-(2-(2-(2-(2-((2-(2,6-dioxopiperidin-3-yl)-1,3-dioxoisoindolin-5-yl)oxy)ethoxy)ethoxy)ethoxy)ethoxy)phenyl)-N-(5-methyl-4-(1-(2-methylbenzoyl)indolin-5-yl)thiazol-2-yl)acetamide

A solution of 2-(2-(2-(2-(3-(2-((5-methyl-4-(1-(2-methylbenzoyl)indolin-5-yl)thiazol-2-yl)amino)-2-oxoethyl)phenoxy)ethoxy)ethoxy)ethoxy)ethyl 4-methylbenzenesulfonate (0.10 g, 0.12 mmol), 2-(2,6-dioxopiperidin-3-yl)-5-hydroxyisoindoline-1,3-dione (0.035 g, 0.12 mmol) and potassium carbonate (0.05 g, 0.36 mmol) in N,N-dimethylformamide (8.0 mL) was stirred at 50° C. for 16 hrs. The reaction mixture was purified by Prep-HPLC (Column: Welch Xtimate 21.2×250 mm C18, 10 μm, Mobile Phase: A: water (10 mmol/L ammonium bicarbonate) B: acetonitrile; B %: 30%-70% in 15.0 min) to give 2-(3-(2-(2-(2-(2-((2-(2,6-dioxopiperidin-3-yl)-1,3-dioxoisoindolin-5-yl)oxy)ethoxy)ethoxy)ethoxy)ethoxy)phenyl)-N-(5-methyl-4-(1-(2-methylbenzoyl)indolin-5-yl)thiazol-2-yl)acetamide (17.1 mg, 15.2% yield) as a white solid. ¹H NMR (400 MHz, DMSO-d₆): δ12.33 & 12.22 (s, 1H)*, 11.11 (s, 1H), 8.23 & 5.45* (d, J=6.8 Hz, 1H), 7.80 (d, J=6.4 Hz, 1H), 7.52 (brs, 1H), 7.52 & 7.01* (brs, 1H), 7.43 (d, J=1.2 Hz, 1H), 7.37-7.30 (m, 5H), 7.23 (t, J=6.2 Hz, 1H), 6.92-6.88 (m, 2H), 6.83 (d, J=5.6 Hz, 1H), 5.11 (dd, J=10.4 & 4.4 Hz, 1H), 4.28 (t, J=3.2 Hz, 2H), 4.21 & 3.70 (m, 2H)*, 4.06 (m, 2H), 3.78-3.70 (m, 6H), 3.56-3.51 (s, 8H), 3.13 (t, J=6.6 Hz, 2H), 2.91-2.84 (m, 1H), 2.60-2.52 (m, 2H), 2.45 & 2.33 (s, 3H)*, 2.29 & 2.18 (s, 3H)*, 2.04-2.00 (m, 1H). MS (ESI) m/z 916.0 [M+H]⁺. *Multiple signals arising from conformational isomers.

### Example 13

### Synthesis of 2-(3-(3-((2-(2,6-dioxopiperidin-3-yl)-1,3-dioxoisoindolin-4-yl)amino)propoxy)phenyl)-N-(5-methyl-4-(1-(2-methylbenzoyl)indolin-5-yl)thiazol-2-yl)acetamide (Cpd. No. 6)

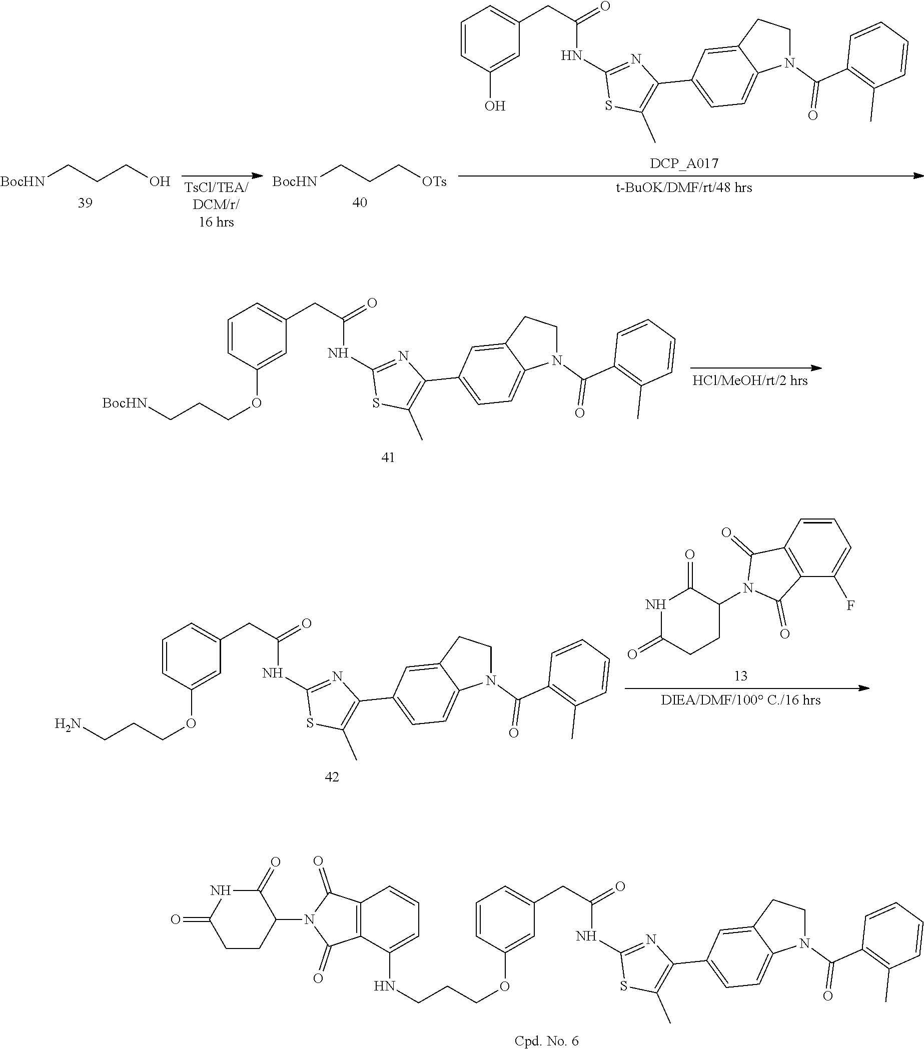

### Step 1. 3-((tert-butoxycarbonyl)amino)propyl 4-methylbenzenesulfonate

A solution of tert-butyl (3-hydroxypropyl)carbamate (1.00 g, 5.71 mmol), tosyl chloride (1.31 g, 6.85 mmol) and triethylamine (1.73 g, 17.12 mmol) in dichloromethane (50 mL) was stirred at room temperature for 16 hrs. The mixture was diluted with water (50 mL), extracted with dichloromethane (100 mL), dried over anhydrous sodium sulfate, filtered and concentrated in vacuo. The residue was purified by flash column chromatography (dichloromethane) to give 3-((tert-butoxycarbonyl)amino)propyl 4-methylbenzenesulfonate (1.0 g, 53% yield) as a yellow oil. MS (ESI) m/z 230.1[M+H−100]⁺

### Step 2. tert-butyl (3-(3-(2-((5-methyl-4-(1-(2-methylbenzoyl)indolin-5-yl)thiazol-2-yl)amino)-2-oxoethyl)phenoxy)propyl)carbamate

A solution of 2-(3-hydroxyphenyl)-N-(5-methyl-4-(1-(2-methylbenzoyl)indolin-5-yl)thiazol-2-yl)acetamide (150 mg, 0.31 mmol), 3-((tert-butoxycarbonyl)amino)propyl 4-methylbenzenesulfonate (203.9 mg, 0.62 mmol), potassium tert-butoxide (104.1 mg, 0.93 mmol) in N,N-dimethylformamide (10 mL) was stirred at room temperature for 48 brs. The mixture was quenched with water (50 mL), extracted with dichloromethane (50 mL×2), dried over anhydrous sodium sulfate, filtered and concentrated in vacuo. The residue was purified by flash column chromatography (ethyl acetate/petroleum ether=3/10) to give crude tert-butyl (3-(3-(2-((5-methyl-4-(1-(2-methylbenzoyl)indolin-5-yl)thiazol-2-yl)amino)-2-oxoethyl)phenoxy)propyl)carbamate (130 mg, 65.4% yield) as a yellow oil. MS (ESI) m/z 641.3 [M+H]⁺.

### Step 3. 2-(3-(3-aminopropoxy)phenyl)-N-(5-methyl-4-(1-(2-methylbenzoyl)indolin-5-yl)thiazol-2-yl)acetamide

A solution of tert-butyl (3-(3-(2-((5-methyl-4-(1-(2-methylbenzoyl)indolin-5-yl)thiazol-2-yl)amino)-2-oxoethyl)phenoxy)propyl)carbamate (130 mg, 0.20 mmol) and hydrochloric acid (4 M in 1,4-dioxane, 5.0 mL) in 1,4-dioxane (2.0 mL) was stirred at room temperature for 2 hrs. The solvent was removed and the residue was dissolved in dichloromethane (100 mL), washed with saturated sodium carbonate solution (20 mL×2), dried over anhydrous sodium, filtered and concentrated in vacuo. The residue was purified by flash column chromatography (dichloromethane/methanol=1/20) to give 2-(3-(3-aminopropoxy)phenyl)-N-(5-methyl-4-(1-(2-methylbenzoyl)indolin-5-yl)thiazol-2-yl)acetamide (90 mg, 82.0%) as a yellow solid. MS (ESI) m/z 541.2 [M+H]⁺.

### Step 4. 2-(3-(3-((2-(2,6-dioxopiperidin-3-yl)-1,3-dioxoisoindolin-4-yl)amino)propoxy)phenyl)-N-(5-methyl-4-(1-(2-methylbenzoyl)indolin-5-yl)thiazol-2-yl)acetamide

A solution of 2-(3-(2-(2-aminoethoxy)ethoxy)phenyl)-N-(5-methyl-4-(1-(2-methylbenzoyl)indolin-5-yl)thiazol-2-yl)acetamide (90 mg, 0.16 mmol), 2-(2,6-dioxopiperidin-3-yl)-4-fluoroisoindoline-1,3-dione (92 mg, 0.32 mmol) and N, N-Diisopropylethylamine (64 mg, 0.48 mmol) in N,N-dimethylformamide (4.0 mL) was stirred at 100° C. for 16 hrs. The reaction mixture was purified by Prep-HPLC (Column: Welch Xtimate 21.2×250 mm C18, 10 μm, Mobile Phase: A: water (10 mmol/L ammonium bicarbonate) B: acetonitrile; B %: 30%-70% in 15.0 min) to give 2-(3-(3-((2-(2,6-dioxopiperidin-3-yl)-1,3-dioxoisoindolin-4-yl)amino)propoxy)phenyl)-N-(5-methyl-4-(1-(2-methylbenzoyl)indolin-5-yl)thiazol-2-yl)acetamide (17.4 mg, 13.1% yield) as a yellow solid. ¹H NMR (400 MHz, 60° C., DMSO-d₆): δ12.07 (s, 1H), 11.91 (s, 1H), 8.24 (brs, 1H), 7.56-7.51 (m, 2H), 7.39-7.28 (m, 5H), 7.22 (t, J=8.0 Hz, 1H), 7.10 (d, J=4.8 Hz, 1H), 7.00 (d, J=7.2 Hz, 1H), 6.99 (s, 1H), 6.91-6.85 (m, 2H), 6.65 (t, J=5.8 Hz, 1H), 5.04-5.00 (m, 1H), 4.09 (t, J=6.2 Hz, 2H), 4.00-4.06 (brs, 2H)*, 3.70 (s, 2H), 3.49 (q, J=3.2 Hz, 2H), 3.14 (m, 2H), 2.91-2.82 (m, 1H), 2.62-2.52 (m, 2H), 2.43 (s, 3H), 2.27 (s, 3H), 2.08-1.99 (m, 3H). MS (ESI) m/z 797.3 [M+H]⁺. *Broad signals that are difficult to see arising from conformational isomers.

### Example 14

### Synthesis of 2-(3-((14-((2-(2,6-dioxopiperidin-3-yl)-1,3-dioxoisoindolin-4-yl)amino)-3,6,9,12-tetraoxatetradecyl)oxy)phenyl)-N-(5-methyl-4-(1-(2-methylbenzoyl)indolin-5-yl)thiazol-2-yl)acetamide (Cpd. No. 4)

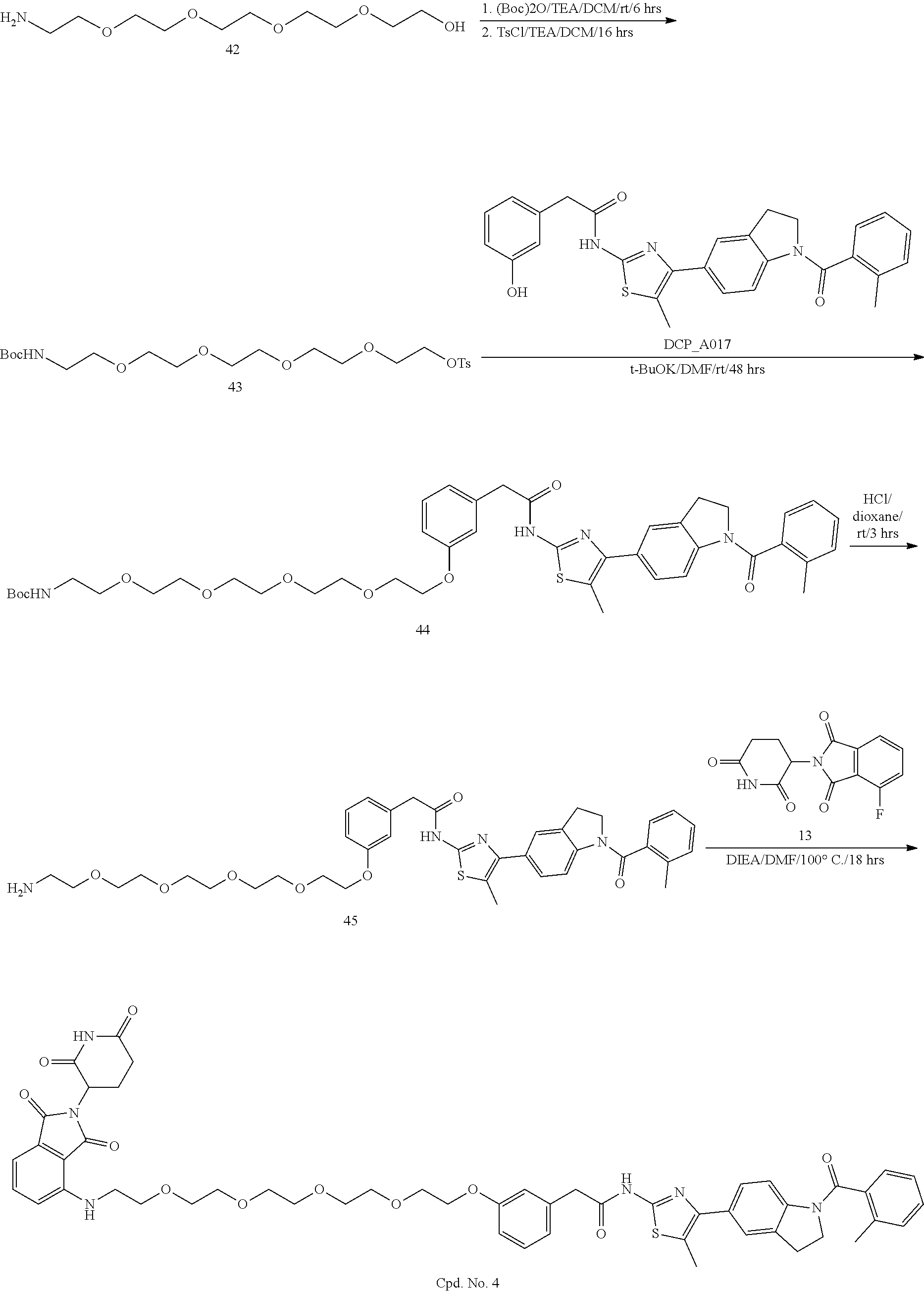

### Step 1. 2,2-dimethyl-4-oxo-3,8,11,14,17-pentaoxa-5-azanonadecan-19-yl 4-methylbenzenesulfonate

A solution of 14-amino-3,6,9,12-tetraoxatetradecan-1-ol (1.0 g, 4.2 mmol), di-tert-butyl decarbonate (0.9 g, 4.2 mmol) and triethylamine (1.28 g, 12.6 mmol) in dichloromethane (10 mL) was stirred at room temperature for 6 hrs. Tosyl chloride (0.8 g, 4.2 mmol) was added and the mixture was stirred for 16 hrs. The mixture was diluted with water (50 mL), extracted with dichloromethane (100 mL), dried over anhydrous sodium sulfate, filtered and concentrated in vacuo. The residue was purified by flash column chromatography (dichloromethane) to give 2,2-dimethyl-4-oxo-3,8,11,14,17-pentaoxa-5-azanonadecan-19-yl 4-methylbenzenesulfonate (0.32 g, 15.4% yield) as a yellow oil. MS (ESI) m/z 392.2 [M+H−100]⁺.

### Step 2. tert-butyl (14-(3-(2-((5-methyl-4-(1-(2-methylbenzoyl)indolin-5-yl)thiazol-2-yl)amino)-2-oxoethyl)phenoxy)-3,6,9,12-tetraoxatetradecyl)carbamate

A solution of 2,2-dimethyl-4-oxo-3,8,11,14,17-pentaoxa-5-azanonadecan-19-yl 4-methylbenzenesulfonate (0.30 g, 0.61 mmol-2-(3-hydroxyphenyl)-N-(5-methyl-4-(1-(2-methylbenzoyl)indolin-5-yl)thiazol-2-yl)acetamide (0.29 g, 0.60 mmol) and potassium tert-butoxide (0.20 g, 1.78 mmol) in N,N-dimethylformamide (15 mL) was stirred at room temperature for 48 hrs. The mixture was quenched with water (50 mL), extracted with dichloromethane (50 mL×2), dried over anhydrous sodium sulfate, filtered and concentrated in vacuo. The residue was purified by flash column chromatography (ethyl acetate/petroleum ether=3/10) to give tert-butyl (14-(3-(2-((5-methyl-4-(1-(2-methylbenzoyl)indolin-5-yl)thiazol-2-yl)amino)-2-oxoethyl)phenoxy)-3,6,9,12-tetraoxatetradecyl)carbamate (0.30 g, 61% yield) as a yellow oil. MS (ESI) m/z 803.5 [M+H]⁺.

### Step 3. 2-(3-((14-amino-3,6,9,12-tetraoxatetradecyl)oxy)phenyl)-N-(5-methyl-4-(1-(2-methylbenzoyl)indolin-5-yl)thiazol-2-yl)acetamide

A solution of tert-butyl (14-(3-(2-((5-methyl-4-(1-(2-methylbenzoyl)indolin-5-yl)thiazol-2-yl)amino)-2-oxoethyl)phenoxy)-3,6,9,12-tetraoxatetradecyl)carbamate (0.28 g, 0.35 mmol) and hydrochloric acid (4 M in 1,4-dioxane, 5.0 mL) in 1,4-dioxane (10.0 mL) was stirred at room temperature for 3 hrs. The solvent was removed and the residue was dissolved in dichloromethane (100 mL), washed with saturated sodium carbonate solution (20 mL×2), dried over anhydrous sodium sulfate, filtered and concentrated. The residue was purified by flash column chromatography (dichloromethane/methanol=1/20) to give 2-(3-((14-amino-3,6,9,12-tetraoxatetradecyl)oxy)phenyl)-N-(5-methyl-4-(1-(2-methylbenzoyl)indolin-5-yl)thiazol-2-yl)acetamide (200 mg, 82% yield) as a white solid. MS (ESI) m/z 703.4 [M+H]⁺.

### Step 4. 2-(3-((14-((2-(2,6-dioxopiperidin-3-yl)-1,3-dioxoisoindolin-4-yl)amino)-3,6,9,12-tetraoxatetradecyl)oxy)phenyl)-N-(5-methyl-4-(1-(2-methylbenzoyl)indolin-5-yl)thiazol-2-yl)acetamide

A solution of 2-(3-((14-amino-3,6,9,12-tetraoxatetradecyl)oxy)phenyl)-N-(5-methyl-4-(1-(2-methylbenzoyl)indolin-5-yl)thiazol-2-yl)acetamide (0.18 g, 0.26 mmol), 2-(2,6-dioxopiperidin-3-yl)-4-fluoroisoindoline-1,3-dione (0.07 g, 0.26 mmol) and N, N-diisopropylethylamine (0.066 g, 0.5 mmol) in N,N-dimethylformamide (2.0 mL) was stirred at 100° C. for 18 hrs. The reaction mixture was purified by Prep-HPLC (Column: Welch Xtimate 21.2×250 mm C18, 10 μm, Mobile Phase: A: water (10 mmol/L ammonium bicarbonate) B: acetonitrile; B %: 30%-70% in 15.0 min) to give 2-(3-((14-((2-(2,6-dioxopiperidin-3-yl)-1,3-dioxoisoindolin-4-yl)amino)-3,6,9,12-tetraoxatetradecyl)oxy)phenyl)-N-(5-methyl-4-(1-(2-methylbenzoyl)indolin-5-yl)thiazol-2-yl)acetamide (29.1 mg, 12% yield) as a yellow solid. ¹H NMR (400 MHz, DMSO-d₆): δ12.34 & 12.27 (s, 1H)*, 11.10 (s, 1H), 8.24 & 5.54* (d, J=8.8 Hz, 1H), 7.59-7.53 (m, 3H), 7.38-7.31 (m, 7.23 (t, J=7.8 Hz, 1H), 7.12 (d, J=7.6 Hz, 1H), 7.03 (d, J=7.2 Hz, 1H), 6.93-6.89 (m, 2H), 6.84 (d, J=8.4 Hz, 1H), 6.60 (t, J=5.4 Hz, 1H), 5.05 (dd, J=13.0 & 5.4 Hz, 1H), 4.21 & 3.70 (m, 2H)*, 4.06 (m, 2H), 3.74-3.71 (m, 3.62-3.43 (m, 16H), 3.13 (t, J=7.6 Hz, 2H), 2.88-2.85 (m, 1H), 2.60-2.56 (m, 2H), 2.42 & 2.34 (s, 3H)*, 2.26 & 2.18 (s, 3H)*, 2.03-2.00 (m, 1H). MS (ESI) m/z 959.5 [M+H]⁺. *Multiple signals arising from conformational isomers.

### Example 15

### Synthesis of 2-(3-((17-((2-(2,6-dioxopiperidin-3-yl)-1,3-dioxoisoindolin-4-yl)amino)-3,6,9,12,15-pentaoxaheptadecyl)oxy)phenyl)-N-(5-methyl-4-(1-(2-methylbenzoyl)indolin-5-yl)thiazol-2-yl)acetamide (Cpd. No. 5)

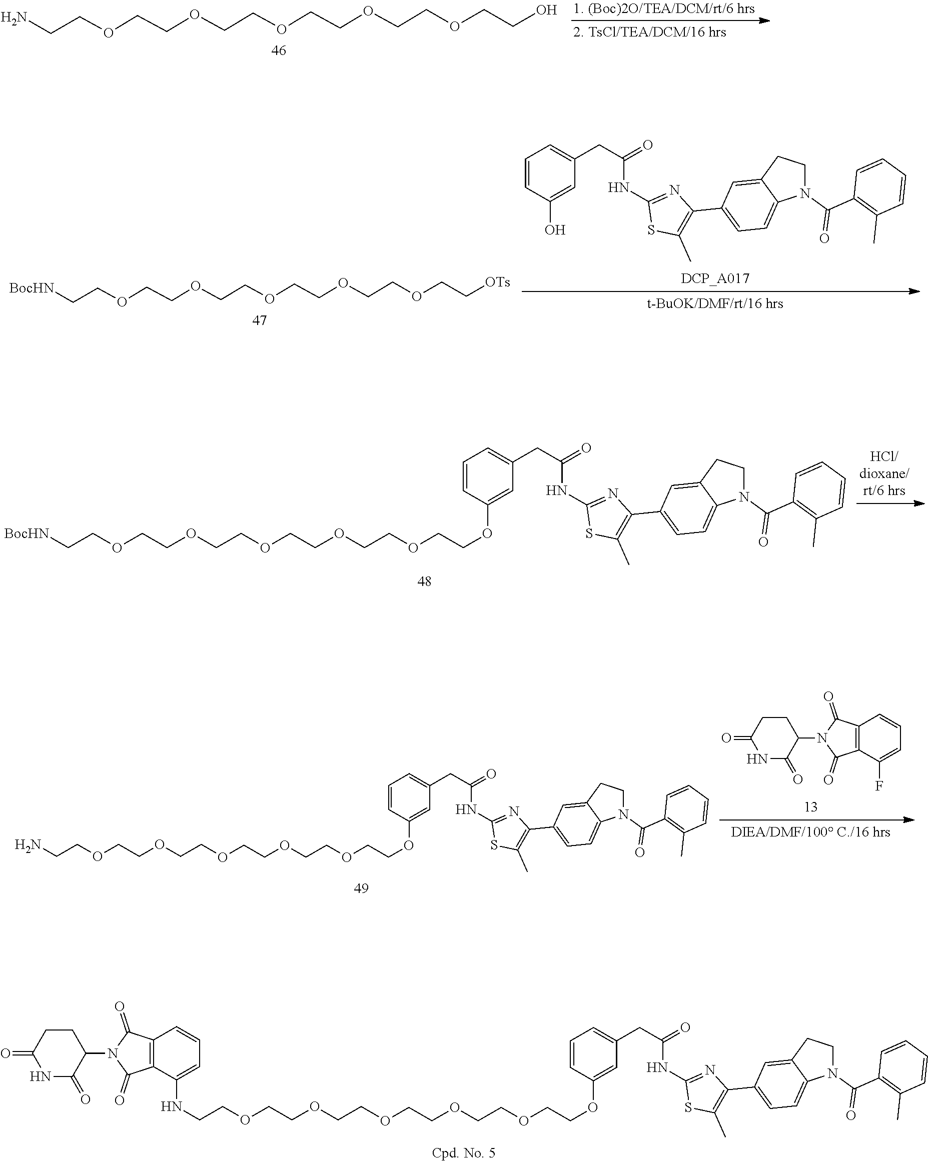

### Step 1. 2,2-dimethyl-4-oxo-3,8,11,14,17,20-hexaoxa-5-azadocosan-22-yl 4-methylbenzenesulfonate

A solution of 17-amino-3,6,9,12,15-pentaoxaheptadecan-1-ol (1.00 g, 3.56 mmol), di-tert-butyl decarbonate (0.92 g, 4.26 mmol) and triethylamine (0.72 g, 7.10 mmol) in dichloromethane (30 mL) was stirred at room temperature for 6 hrs. Tosyl chloride (1.01 g, 5.32 mmol) was added and the mixture was stirred for 16 hrs. The mixture was diluted with water (50 mL), extracted with dichloromethane (100 mL×3), dried over anhydrous sodium sulfate, filtered and concentrated in vacuo. The residue was purified by flash column chromatography (dichloromethane) to give 2,2-dimethyl-4-oxo-3,8,11,14,17,20-hexaoxa-5-azadocosan-22-yl 4-methylbenzenesulfonate (0.55 g, 28.9% yield) as a yellow oil. MS (ESI) m/z 436.0 [M+H−100]⁺.

### Step 2. tert-butyl (17-(3-(2-((5-methyl-4-(1-(2-methylbenzoyl)indolin-5-yl)thiazol-2-yl)amino)-2-oxoethyl)phenoxy)-3,6,9,12,15-pentaoxaheptadecyl)carbamate

A solution of 2,2-dimethyl-4-oxo-3,8,11,14,17,20-hexaoxa-5-azadocosan-22-yl 4-methylbenzenesulfonate (550 mg, 1.03 mmol), 2-(3-hydroxyphenyl)-N-(5-methyl-4-(1-(2-methylbenzoyl)indolin-5-yl)thiazol-2-yl)acetamide (589 mg, 1.22 mmol), potassium tert-butoxide (346 mg, 3.08 mmol) in N,N-dimethylfornamide (10 mL) was stirred at room temperature for 16 hrs. The mixture was quenched with water (50 ml), extracted with dichloromethane (50 mL×3), dried over anhydrous sodium sulfate, filtered and concentrated in vacuo. The residue was purified by flash column chromatography (ethyl acetate/petroleum ether=3/10) to give tert-butyl (17-(3-(2-((5-methyl-4-(1-(2-methylbenzoyl)indolin-5-yl)thiazol-2-yl)amino)-2-oxoethyl)phenoxy)-3,6,9,12,15-pentaoxaheptadecyl)carbamate (360 mg, 41.4% yield) as a yellow solid. MS (ESI) m/z 847.2 [M+H]⁺.

### Step 3. 2-(3-((17-amino-3,6,9,12,15-pentaoxaheptadecyl)oxy)phenyl)-N-(5-methyl-4-(1-(2-methylbenzoyl)indolin-5-yl)thiazol-2-yl)acetamide

A solution of tert-butyl (17-(3-(2-((5-methyl-4-(1-(2-methylbenzoyl)indolin-5-yl)thiazol-2-yl)amino)-2-oxoethyl)phenoxy)-3,6,9,12,15-pentaoxaheptadecyl)carbamate (0.36 g, 0.42 mmol) and hydrochloric acid (4 M in 1,4-dioxane, 5.0 mL) in 1,4-dioxane (3.0 mL) was stirred at room temperature for 6 hrs. The solvent was removed and the residue was dissolved in dichloromethane (100 mL), washed with saturated sodium carbonate solution (20 mL×2), dried over anhydrous sodium, filtered and concentrated in vacuo. The residue was purified by flash column chromatography (dichloromethane/methanol=1/20) to give 2-(3-((17-amino-3,6,9,12,15-pentaoxaheptadecyl)oxy)phenyl)-N-(5-methyl-4-(1-(2-methylbenzoyl)indolin-5-yl)thiazol-2-yl)acetamide (0.19 g, 60% yield) as a white solid. MS (ESI) m/z 748.3[M+H]⁺.

### Step 4. 2-(3-((17-((2-(2,6-dioxopiperidin-3-yl)-1,3-dioxoisoindolin-4-yl)amino)-3,6,9,12,15-pentaoxaheptadecyl)oxy)phenyl)-N-(5-methyl-4-(1-(2-methylbenzoyl)indolin-5-yl)thiazol-2-yl)acetamide

A solution of 2-(3-((17-amino-3,6,9,12,15-pentaoxaheptadecyl)oxy)phenyl)-N-(5-methyl-4-(1-(2-methylbenzoyl)indolin-5-yl)thiazol-2-yl)acetamide (0.19 g, 0.25 mmol), 2-(2,6-dioxopiperidin-3-yl)-4-fluoroisoindoline-1,3-dione (0.15 g, 0.54 mmol) and N, N-diisopropylethylanine (0.04 g, 0.30 mmol) in N,N-dimethylformamide (3.0 mL) was stirred at 100° C. for 16 hrs. The reaction mixture was purified by Prep-HPLC (Column: Welch Xtimate 21.2×250 mm C18, 10 μm, Mobile Phase: A: water (10 mmol/L ammonium bicarbonate) B: acetonitrile; B %: 30%-70% in 15.0 min) to give 2-(3-((17-((2-(2,6-dioxopiperidin-3-yl)-1,3-dioxoisoindolin-4-yl)amino)-3,6,9,12,15-pentaoxa heptadecyl)oxy)phenyl)-N-(5-methyl-4-(1-(2-methylbenzoyl)indolin-5-yl)thiazol-2-yl)acetamide (25 mg, 10% yield) as a yellow solid. ¹H NMR (400 MHz, DMSO-d₆): δ12.34 & 12.31 (s, 1H)*, 11.10 (s, 1H), 8.24 & 5.54* (d, J=8.8 Hz, 1H), 7.58-7.52 (m, 3H), 7.37-7.34 (m, 7.23 (t, J=6.8 Hz, 1H), 7.12 (d, J=6.8 Hz, 1H), 7.03 (d, J=6.8 Hz, 1H), 6.93-6.89 (m, 2H), 6.84 (d, 1=7.2 Hz, 1H), 6.60 (t, 1=5.4 Hz, 1H), 5.05 (dd, J=12.8 & 5.8 Hz, 1H), 4.21 & 3.70 (m, 2H)*, 4.06 (m, 2H), 3.74-3.70 (m, 3.61-3.44 (m, 20H), 3.13 (t, J=4.2 Hz, 2H), 2.92-2.83 (m, 1H), 2.60-2.54 (m, 2H), 2.44 & 2.34 (s, 3H)*, 2.29 & 2.18 (s, 3H)*, 2.05-1.99 (m, 1H). MS (ESI) m/z 1003.5[M+H]⁺. *Multiple signals arising from conformational isomers.

### Example 16

### Synthesis of 2-(3-((14-((2-(2,6-dioxopiperidin-3-yl)-1,3-dioxoisoindolin-4-yl)oxy)-3,6,9,12-tetraoxatetradecyl)oxy)phenyl)-N-(5-methyl-4-(1-(2-methylbenzoyl)indolin-5-yl)thiazol-2-yl)acetamide (Cpd. No. 9)

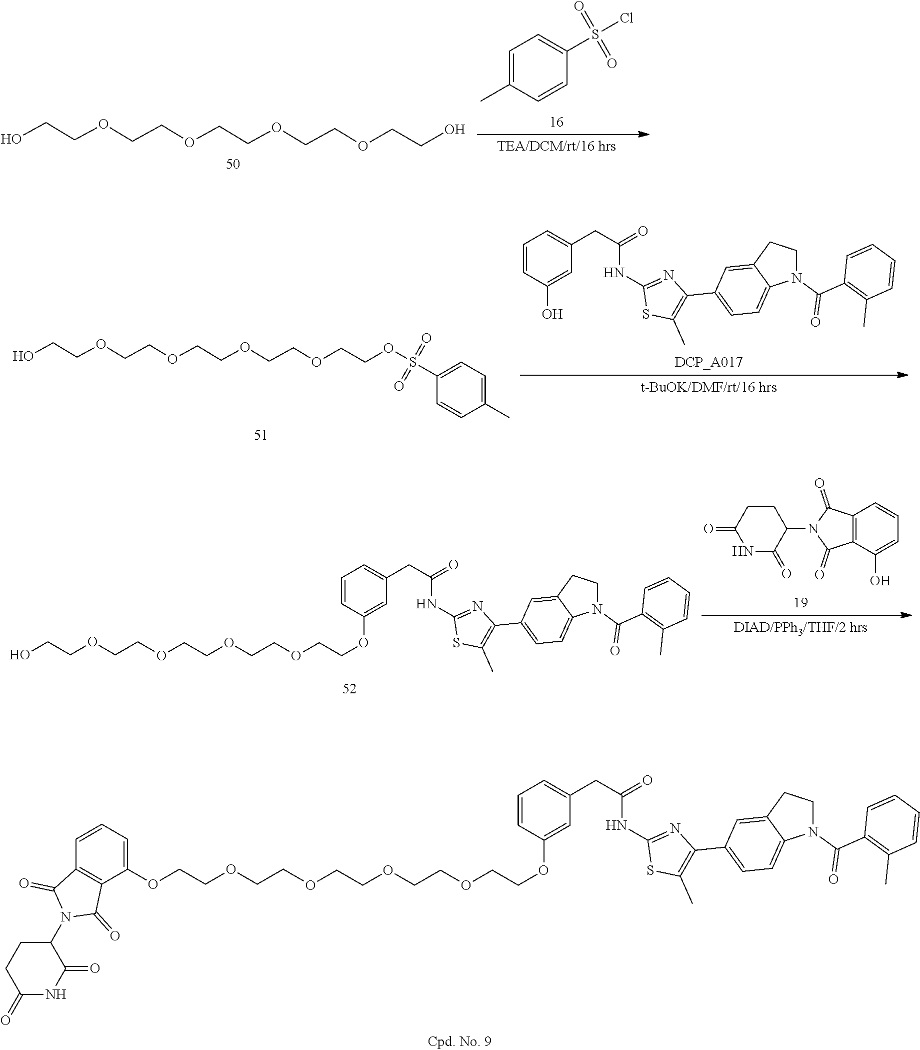

### Step 1. 14-hydroxy-3,6,9,12-tetraoxatetradecyl 4-methylbenzenesulfonate

A solution of 3,6,9,12-tetraoxatetradecane-1,14-diol (5.6 g, 23.5 mmol), triethylamine (6.0 g, 59 mmol), 4-methylbenzenesulfonyl chloride (1.9 g, 10 mmol) in dichloromethane (200 mL) was stirred at room temperature for 16 hrs. The mixture was quenched with water (50 mL), extracted with ethyl acetate (50 mL×2), dried over anhydrous sodium sulfate, filtered and concentrated in vacuo. The residue was purified by flash column chromatography (ethyl acetate/petroleum ether=1:1) to give 14-hydroxy-3,6,9,12-tetraoxatetradecyl 4-methylbenzenesulfonate (3.9 g, 98% yield as a yellow oil. MS (ESI) m/z 393.3 [M+H]⁺.

### Step 2. 2-(3-((14-hydroxy-3,6,9,12-tetraoxatetradecyl)oxy)phenyl)-N-(5-methyl-4-(1-(2-methylbenzoyl)indolin-5-yl)thiazol-2-yl)acetamide

A mixture of 14-hydroxy-3,6,9,12-tetraoxatetradecyl 4-methylbenzenesulfonate (0.30 g, 0.76 mmol), 2-(3-hydroxyphenyl)-N-(5-methyl-4-(1-(2-methylbenzoyl)indolin-5-yl)thiazol-2-yl)acetamide (0.33 g, 0.68 mmol), potassium tert-butoxide (0.22 g, 1.96 mmol) in N,N-dimethylformamide (20 mL) was stirred at room temperature for 16 hrs. The reaction mixture was quenched with water (40 mL), extracted with ethyl acetate (50 nL×2), dried over sodium sulfate, concentrated in vacuo. The residue was purified by flash chromatography (ethyl acetate/petroleum ether=1/1) to give 2-(3-((14-hydroxy-3,6,9,12-tetraoxatetradecyl)oxy)phenyl)-N-(5-methyl-4-(1-(2-methylbenzoyl)indolin-5-yl)thiazol-2-yl)acetamide (0.25 g, 52% yield) as a white solid. MS (ESI) m/z 704.4 [M+H]⁺.

### Step 3. 2-(3-((14-((2-(2,6-dioxopiperidin-3-yl)-1,3-dioxoisoindolin-4-yl)oxy)-3,6,9,12-tetraoxatetradecyl)oxy)phenyl)-N-(5-methyl-4-(1-(2-methylbenzoyl)indolin-5-yl)thiazol-2-yl)acetamide

To a solution of 2-(3-((14-hydroxy-3,6,9,12-tetraoxatetradecyl)oxy)phenyl)-N-(5-methyl-4-(1-(2-methylbenzoyl)indolin-5-yl)thiazol-2-yl)acetamide (100 mg, 0.14 mmol), 2-(2,6-dioxopiperidin-3-yl)-4-hydroxyisoindoline-1,3-dione (49.0 mg, 0.18 mmol) and triphenylphosphine (89.0 mg, 0.34 mmol) in tetrahydrofuran (5.0 mL), was added diisopropylazodicarboxylate (49.0 mg, 0.24 mmol) at room temperature. The mixture was stirred at room temperature for 2 hrs. The mixture was diluted with water (10 mL), extracted with dichloromethane (50 mL), dried over anhydrous sodium sulfate, filtered and concentrated in vacuo. The residue was purified by Prep-HPLC (Column: Welch Xtimate 21.2×250 mm C18, 10 μm, Mobile Phase: A: water (10 mmol/L ammonium bicarbonate) B: acetonitrile; B %: 30%-70% in 15.0 min) to give 2-(3-((14-((2-(2,6-dioxopiperidin-3-yl)-1,3-dioxoisoindolin-4-yl)oxy)-3,6,9,12-tetraoxatetradecyl)oxy)phenyl)-N-(5-methyl-4-(1-(2-methylbenzoyl)indolin-5-yl)thiazol-2-yl)acetamide (52 mg, 39% yield) as white solid. ¹H NMR (400 MHz, DMSO-d₆): δ12.22 (bs, 1H), 11.11 (s, 1H), 8.23 & 5.56* (d, J=8.4 Hz, 1H), 7.78 (t, J=7.8 Hz, 1H), 7.52-7.49 (m, 2H), 7.52 &7.03* (m, 1H), 7.44 (d, 1=7.2 Hz, 1H), 7.37-7.30 (m, 4H), 7.23 (t, J=7.8 Hz, 1H), 6.92-6.89 (m, 2H), 6.83 (d, J=7.6 Hz, 1H), 5.08 (dd, J=12.8 & 5.6 Hz, 1H), 4.32 (t, J=4.2 Hz, 2H), 4.21 & 3.72 (m, 2H)*, 4.06 (t, J=4.0 Hz, 2H), 3.79 (t, J=4.4 Hz, 2H), 3.73-3.70 (m, 3.63 (t, J=4.8 Hz, 2H), 3.57-3.56 (m, 2H), 3.53-3.50 (m, 8H), 3.13 (t, J=7.8 Hz, 2H), 2.95-2.81 (m, 1H), 2.60-2.53 (m, 2H), 2.45 & 2.33 (s, 3H)*, 2.29 & 2.18 (s, 3H)*, 2.03-2.00 (m, 1H). MS (ESI) m/z 480.9 [M/2+H]⁺. *Multiple signals arising from conformational isomers.

### Example 17

### Synthesis of 2-(3-((17-((2-(2,6-dioxopiperidin-3-yl)-1,3-dioxoisoindolin-4-yl)oxy)-3,6,9,12,15-pentaoxaheptadecyl)oxy)phenyl)-N-(5-methyl-4-(1-(2-methylbenzoyl)indolin-5-yl)thiazol-2-yl)acetamide (Cpd. No. 10)

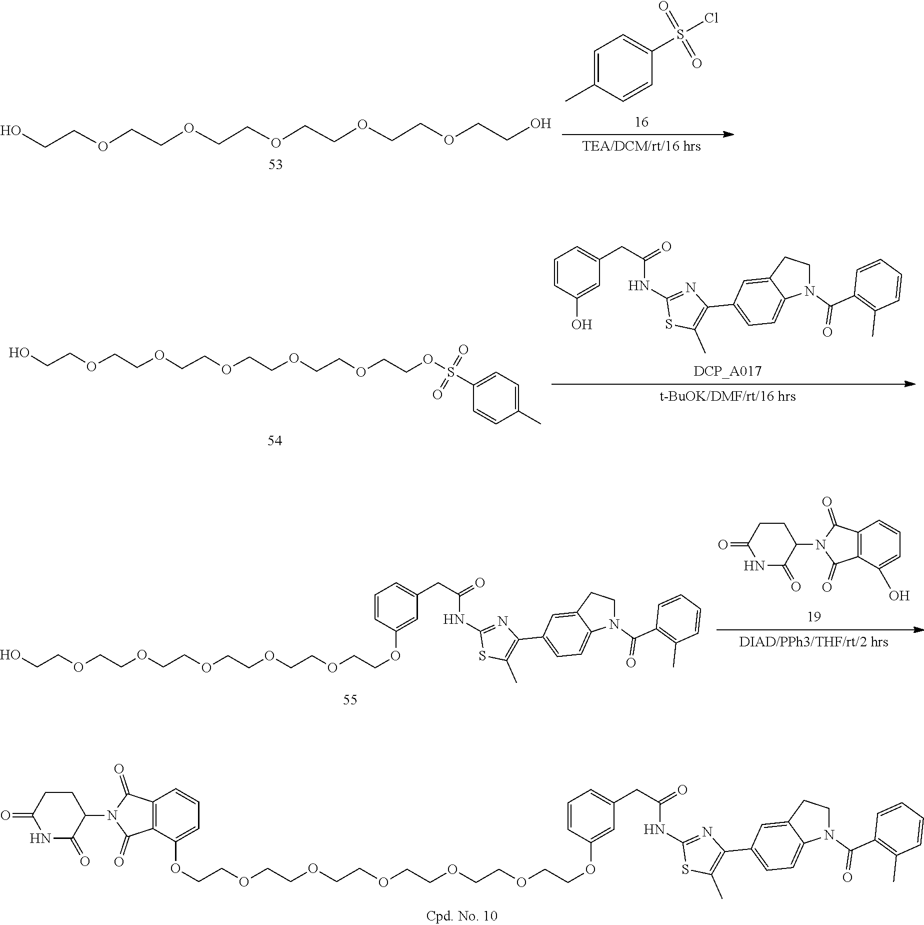

### Step 1. 17-hydroxy-3,6,9,12,15-pentaoxaheptadecyl 4-methylbenzenesulfonate

A solution of 3,6,9,12,15-pentaoxaheptadecane-1,17-diol (5.6 g, 20 mmol), triethylamine (6.0 g, 59 mmol) and 4-methylbenzenesulfonyl chloride (1.9 g, 10 mmol) in dichlorourethane (60 mL). The mixture was stirred at room temperature for 16 hrs. The mixture was quenched with water (50 mL), then extracted with ethyl acetate (50 mL×2), dried over anhydrous sodium sulfate, filtered and concentrated in vacuo. The residue was purified by flash column chromatography (ethyl acetate/petroleum ether=2:5) to give 17-hydroxy-3,6,9,12,15-pentaoxaheptadecyl 4-methylbenzenesulfonate (4.3 g, 98.5% yield) as a yellow oil. MS (ESI) m/z 437.3 [M+H]⁺.

### Step 2. 2-(3-((17-hydroxy-3,6,9,12,15-pentaoxaheptadecyl)oxy)phenyl)-N-(5-methyl-4-(1-(2-methylbenzoyl)indolin-5-yl)thiazol-2-yl)acetamide

A mixture of 17-hydroxy-3,6,9,12,15-pentaoxaheptadecyl 4-methylbenzenesulfonate (0.30 g, 0.69 mmol), 2-(3-hydroxyphenyl)-N-(5-methyl-4-(1-(2-methylbenzoyl)indolin-5-yl)thiazol-2-yl)acetamide (0.39 g, 0.81 mmol), potassium t-butoxide (0.15 g, 1.38 mmol) in dry N,N-dimethylformamide (10 mL) was stirred at room temperature for 16 hrs. The reaction mixture was quenched with water (50 mL) and extracted with ethyl acetate (30 mL×2), dried over sodium sulfate, filtered and concentrated in vacuo. The residue was purified by flash column chromatography (ethyl acetate/petroleum ether 1:1) to give 2-(3-((17-hydroxy-3,6,9,12,15-pentaoxaheptadecyl)oxy)phenyl)-N-(5-methyl-4-(1-(2-methylbenzoyl)indolin-5-yl)thiazol-2-yl)acetamide (0.25 g, 48.6% yield) as a white solid. MS (ESI) m/z 748.3 [M+H]⁺.

### Step 3. 2-(3-((17-((2-(2,6-dioxopiperidin-3-yl)-1,3-dioxoisoindolin-4-yl)oxy)-3,6,9,12,15-pentaoxaheptadecyl)oxy)phenyl)-N-(5-methyl-4-(1-(2-methylbenzoyl)indolin-5-yl)thiazol-2-yl)acetamide

To a solution of 2-(3-((14-hydroxy-3,6,9,12-tetraoxatetradecyl)oxy)phenyl)-N-(5-methyl-4-(1-(2-methylbenzoyl)indolin-5-yl)thiazol-2-yl)acetamide (0.25 g, 0.33 mmol), 2-(2,6-dioxopiperidin-3-yl)-4-hydroxyisoindoline-1,3-dione (0.11 g, 0.39 mmol) and triphenylphosphine (0.17 g, 0.66 mmol) in tetrahydrofuran (8.0 mL), was added diisopropylazodicarboxylate (0.079 g, 0.39 mmol) at room temperature. The mixture was stirred at room temperature for 2 hrs. The mixture was diluted with water (20 mL), extracted with ethyl acetate (20 mL×2), dried over anhydrous sodium sulfate, filtered and concentrated in vacuo. The residue was purified by Prep-HPLC (Column: Welch Xtimate 21.2×250 mm C18, 10 μm, Mobile Phase: A: water (10 mmol/L ammonium bicarbonate) B: acetonitrile; B %: 30%-70% in 15.0 min) to give 2-(3-((17-((2-(2,6-dioxopiperidin-3-yl)-1,3-dioxoisoindolin-4-yl)oxy)-3,6,9,12,15-pentaoxaheptadecyl) oxy)phenyl)-N-(5-methyl-4-(1-(2-methylbenzoyl)indolin-5-yl)thiazol-2-yl)acetamide (6.0 mg, 1.8% yield) as a white solid. ¹H NMR (400 MHz, 60° C., DMSO-d₆): δ12.11 (bs, 1H), 10.9 (s, 1H), 7.77 (t, J=7.8 Hz, 1H), 7.52-7.48 (m, 2H), 7.43 (d, J=7.2 Hz, 1H), 7.37-7.28 (m, 6H), 7.21 (t, J=7.8 Hz, 1H), 6.92-6.88 (m, 2H), 6.82 (d, J=8.0 Hz, 1H), 5.04 (dd, J=12.4 & 5.6 Hz, 1H), 4.33 (t, J=4.6 Hz, 2H), 4.07 (t, J=4.8 Hz, 2H), 3.79 (t, J=4.6 Hz, 2H), 3.74-3.70 (m, 5H), 3.62 (t, J=4.6 Hz, 2H), 3.70 (m, 2H), 3.53-3.49 (m, 13H), 3.11 (m, 2H), 2.91-2.82 (m, 1H), 2.60-2.51 (m, 2H), 2.42 (s, 3H), 2.26 (s, 3H), 2.06-2.99 (m, 1H). MS (ESI) m/z 1005.4 [M+H]⁺.

### Example 18

### Synthesis of 2-(3-((14-((2-(2,6-dioxopiperidin-3-yl)-1,3-dioxoisoindolin-5-yl)oxy)-3,6,9,12-tetraoxatetradecyl)oxy)phenyl)-N-(5-methyl-4-(1-(2-methylbenzoyl)indolin-5-yl)thiazol-2-yl)acetamide (Cpd. No. 14)

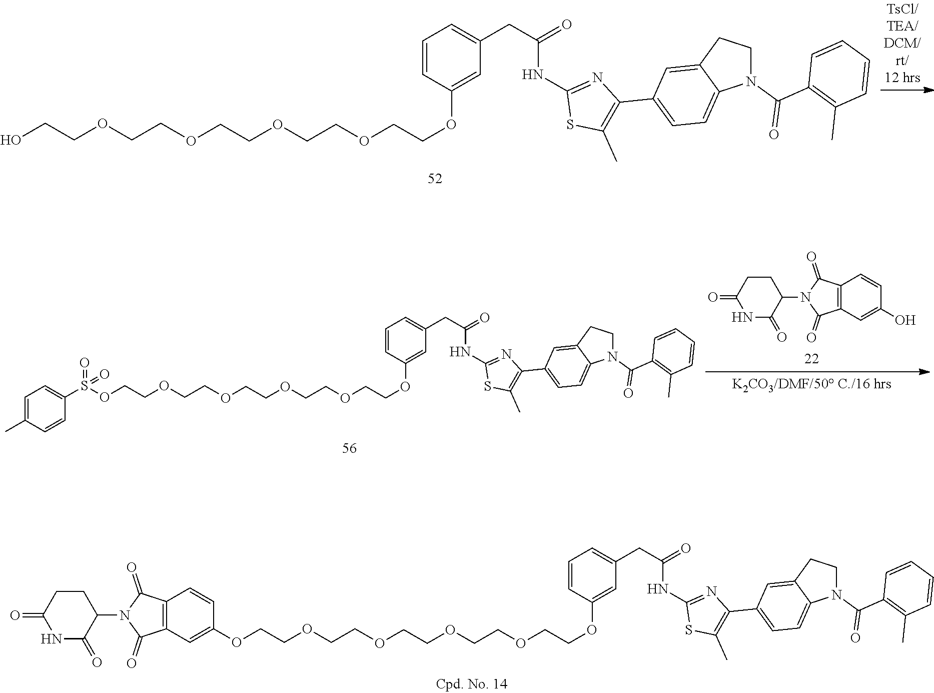 [SMILES: Cc1ccccc1C(=O)N1CCc2cc(-c3nc(NC(=O)Cc4cccc(OCCOCCOCCOCCOCCO)c4)sc3C)ccc21 ; Cc1ccc(S(=O)(=O)OCCOCCOCCOCCOCCOc2cccc(CC(=O)Nc3nc(-c4ccc5c(c4)CCN5C(=O)c4ccccc4C)c(C)s3)c2)cc1 ; O=C1CCC(N2C(=O)c3ccc(O)cc3C2=O)C(=O)N1 ; Cc1ccccc1C(=O)N1CCc2cc(-c3nc(NC(=O)Cc4cccc(OCCOCCOCCOCCOCCOc5ccc6c(c5)C(=O)N(C5CCC(=O)NC5=O)C6=O)c4)sc3C)ccc21]

### Step 1. 14-(3-(2-((5-methyl-4-(1-(2-methylbenzoyl)indolin-5-yl)thiazol-2-yl)amino)-2-oxoethyl)phenoxy)-3,6,9,12-tetraoxatetradecyl 4-methylbenzenesulfonate PGP-166

A solution of 2-(3-((14-hydroxy-3,6,9,12-tetraoxatetradecyl)oxy)phenyl)-N-(5-methyl-4-(1-(2-methylbenzoyl)indolin-5-yl)thiazol-2-yl)acetamide (0.29 g, 0.41 mmol), tosyl chloride (0.079 g, 0.41 mmol) and triethylamine (0.13 g, 1.23 mmol) in dichloromethane (20 mL) was stirred at room temperature for 12 hrs. The mixture was diluted with water (10 mL), extracted with dichloromethane (30 mL), dried over anhydrous sodium sulfate, filtered and concentrated in vacuo. The residue was purified by flash column chromatography (dichloromethane) to give 14-(3-(2-((5-methyl-4-(1-(2-methylbenzoyl)indolin-5-yl)thiazol-2-yl)amino)-2-oxoethyl)phenoxy)-3,6,9,12-tetraoxatetradecyl 4-methylbenzenesulfonate (0.15 g, 43% yield) as a white solid. MS (ESI) m/z 858.3 [M+H]⁺.

### Step 2. 2-(3-((14-((2-(2,6-dioxopiperidin-3-yl)-1,3-dioxoisoindolin-5-yl)oxy)-3,6,9,12-tetraoxatetradecyl)oxy)phenyl)-N-(5-methyl-4-(1-(2-methylbenzoyl)indolin-5-yl)thiazol-2-yl)acetamide

A solution of 14-(3-(2-((5-methyl-4-(1-(2-methylbenzoyl)indolin-5-yl)thiazol-2-yl)amino)-2-oxoethyl)phenoxy)-3,6,9,12-tetraoxatetradecyl 4-methylbenzenesulfonate (0.15 g, 0.17 mmol), 2-(2,6-dioxopiperidin-3-yl)-5-hydroxyisoindoline-1,3-dione (0.05 g, 0.18 mmol) and potassium carbonate (0.07 g, 0.51 mmol) in N,N-dimethylformamide (8.0 mL) was stirred at 50° C. for 16 hrs. The reaction mixture was purified by Prep-HPLC (Column: Welch Xtimate 21.2×250 mm C18, 10 μm, Mobile Phase: A: water (10 mmol/L ammonium bicarbonate) B: acetonitrile; B %: 30%-70% in 15.0 min) to give 2-(3-((14-((2-(2,6-dioxopiperidin-3-yl)-1,3-dioxoisoindolin-5-yl)oxy)-3,6,9,12-tetraoxatetradecyl)oxy)phenyl)-N-(5-methyl-4-(1-(2-methylbenzoyl)indolin-5-yl)thiazol-2-yl)acetamide (10.1 mg, 6.2% yield) as a white solid. ¹H NMR (400 MHz, DMSO-d₆): δ12.33 & 12.31 (s, 1H)*, 11.11 (s, 1H), 8.24 & 5.45* (d, J=9.2 Hz, 1H), 7.81 (d, J=8.4 Hz, 1H), 7.53 (brs, 1H), 7.53 & 7.01* (brs, 1H), 7.44 (d, J=2.4 Hz, 1H), 7.38-7.30 (m, 5H), 7.23 (t, J=8.0 Hz, 1H), 6.92-6.89 (m, 2-1), 6.84 (d, J=7.2 Hz, 1H), 5.12 (dd, J=13.0 & 5.4 Hz, 1H), 4.29 (t, J=4.2 Hz, 2H), 4.21 & 3.70 (m, 2H)*, 4.07 (t, J=4.4 Hz, 2H), 3.78-3.70 (m, 6H), 3.59-3.51 (s, 12H), 3.13 (t, J=7.6 Hz, 2H), 2.90-2.88 (m, 1H), 2.55-2.52 (m, 2H), 2.45 & 2.33 (s, 3H)*, 2.29 & 2.18 (s, 3H)*, 2.05-2.01 (m, 1H). MS(ESI) m/z/2 480.9 [M+H]⁺. *Multiple signals arising from conformational isomers.

### Example 19

### Synthesis of 2-(3-((17-((2-(2,6-dioxopiperidin-3-yl)-1,3-dioxoisoindolin-5-yl)oxy)-3,6,9,12,15-pentaoxaheptadecyl)oxy)phenyl)-N-(5-methyl-4-(1-(2-methylbenzoyl)indolin-5-yl)thiazol-2-yl)acetamide (Cpd. No. 15)

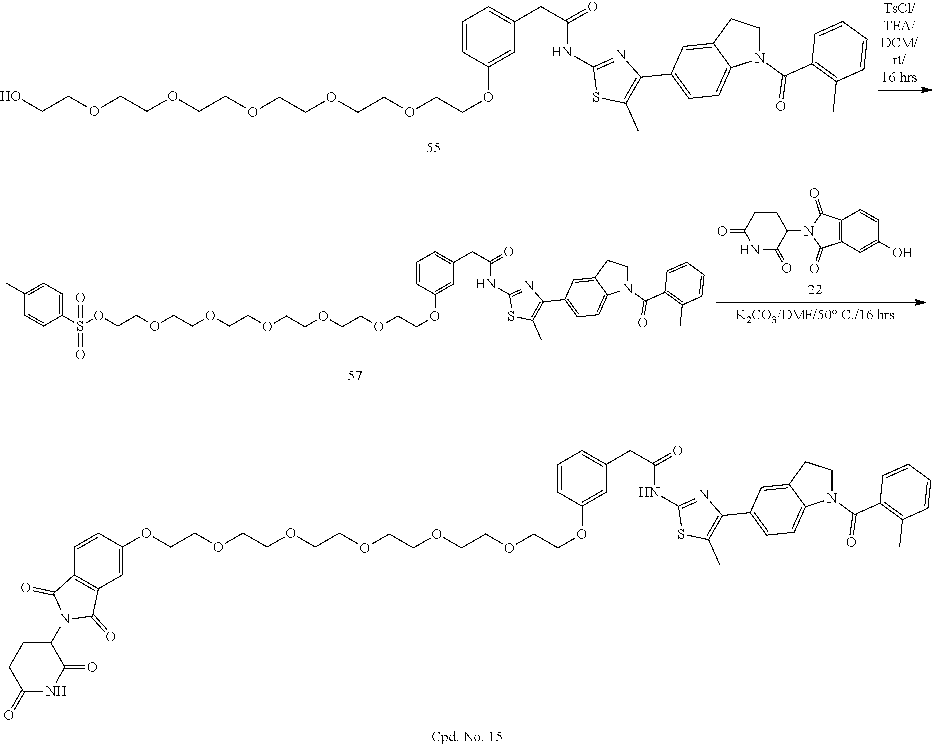 [SMILES: Cc1ccccc1C(=O)N1CCc2cc(-c3nc(NC(=O)Cc4cccc(OCCOCCOCCOCCOCCOCCO)c4)sc3C)ccc21 ; Cc1ccc(S(=O)(=O)OCCOCCOCCOCCOCCOCCOc2cccc(CC(=O)Nc3nc(-c4ccc5c(c4)CCN5C(=O)c4ccccc4C)c(C)s3)c2)cc1 ; O=C1CCC(N2C(=O)c3ccc(O)cc3C2=O)C(=O)N1 ; Cc1ccccc1C(=O)N1CCc2cc(-c3nc(NC(=O)Cc4cccc(OCCOCCOCCOCCOCCOCCOc5ccc6c(c5)C(=O)N(C5CCC(=O)NC5=O)C6=O)c4)sc3C)ccc21]

### Step 1. 17-(3-(2-((5-methyl-4-(1-(2-methylbenzoyl)indolin-5-yl)thiazol-2-yl)amino)-2-oxoethyl)phenoxy)-3,6,9,12,15-pentaoxaheptadecyl 4-methylbenzenesulfonate

A solution of 2-(3-((17-hydroxy-3,6,9,12,15-pentaoxaheptadecyl)oxy)phenyl)-N-(5-methyl-4-(1-(2-methylbenzoyl)indolin-5-yl)thiazol-2-yl)acetamide (0.25 g, 0.33 mmol), triethylamine (0.10 g, 1.0 mmol) and tosyl chloride (0.064 g, 0.33 mmol) in dichloromethane (15 mL) was stirred room temperature for 16 hrs. The mixture was quenched with water (10 mL), extracted with ethyl acetate (50 mL×2), dried over sodium sulfate, concentrated in vacuo. The residue was purified by flash column chromatography (ethyl acetate/petroleum ether=1:1) to give 17-(3-(2-((5-methyl-4-(1-(2-methylbenzoyl)indolin-5-yl)thiazol-2-yl)amino)-2-oxoethyl)phenoxy)-3,6,9,12,15-pentaoxaheptadecyl 4-methylbenzenesulfonate (0.15 g, 49.8%) as a white solid. MS (ESI) m/z 902.3 [M+H]⁺.

### Step 2. 2-(3-((17-((2-(2,6-dioxopiperidin-3-yl)-1,3-dioxoisoindolin-5-yl)oxy)-3,6,9,12,15-pentaoxaheptadecyl)oxy)phenyl)-N-(5-methyl-4-(1-(2-methylbenzoyl)indolin-5-yl)thiazol-2-yl)acetamide

A solution of 17-(3-(2-((5-methyl-4-(1-(2-methylbenzoyl)indolin-5-yl)thiazol-2-yl)amino)-2-oxoethyl)phenoxy)-3,6,9,12,15-pentaoxaheptadecyl 4-methylbenzenesulfonate (130 mg, 0.14 mmol) and 2-(2,6-dioxopiperidin-3-yl)-5-hydroxyisoindoline-1,3-dione (56 mg, 0.2 mmol), potassium carbonate (59.0 mg, 0.42 mmol) in N,N-dimethylfornamide (8.0 mL). The reaction mixture was stirred at 50° C. for 16 hrs. The reaction mixture was purified by Prep-HPLC (Column: Welch Xtimate 21.2×250 mm C18, 10 μm, Mobile Phase: A: water (10 mmol/L ammonium bicarbonate) B: acetonitrile; B %: 30%-70% in 15.0 min) to give 2-(3-((17-((2-(2,6-dioxopiperidin-3-yl)-1,3-dioxoisoindolin-5-yl)oxy)-3,6,9,12,15-pentaoxaheptadecyl)oxy)phenyl)-N-(5-methyl-4-(1-(2-methylbenzoyl)indolin-5-yl)thiazol-2-yl)acetamide (14.2 mg, 10% yield) as a white solid. ¹H NMR (400 MHz, 60° C., DMSO-d₆): δ12.15 (brs, 1H), 10.98 (s, 1H), 8.30 & 8.20* (brs, 1H), 7.81 (d, J=8.4 Hz, 1H), 7.53 (s, 1H), 7.43 (s, 1H), 7.38-7.30 (m, 6H), 7.23 (t, J=8.0 Hz, 1H), 6.92-6.89 (m, 2H), 6.84 (d, J=7.2 Hz, 1H), 5.09 (dd, J=12.8 & 5.6 Hz, 1H), 4.31 (t, J=4.6 Hz, 2H), 4.09 (t, J=4.8 Hz, 2H), 3.80-3.72 (m, 8H), 3.60-3.52 (s, 16H), 3.13-3.11 (m, 2H), 2.90-2.86 (m, 1H), 2.63-2.52 (m, 2H), 2.44 (s, 3H), 2.28 (s, 3H), 2.08-2.00 (m, 1H). MS (ESI) m/z/2: 503.0 [M+H]⁺. *Multiple signals arising from conformational isomers which are very broad and difficult to see.

### Example 20

### Synthesis of N-(4-(1-(cyclopropanecarbonyl)indolin-5-yl)-5-methylthiazol-2-yl)-2-(3-(2-(2-((2-(2,6-dioxopiperidin-3-yl)-1,3-dioxoisoindolin-5-yl)amino)ethoxy)ethoxy)phenyl)acetamide (Cpd. No. 22)

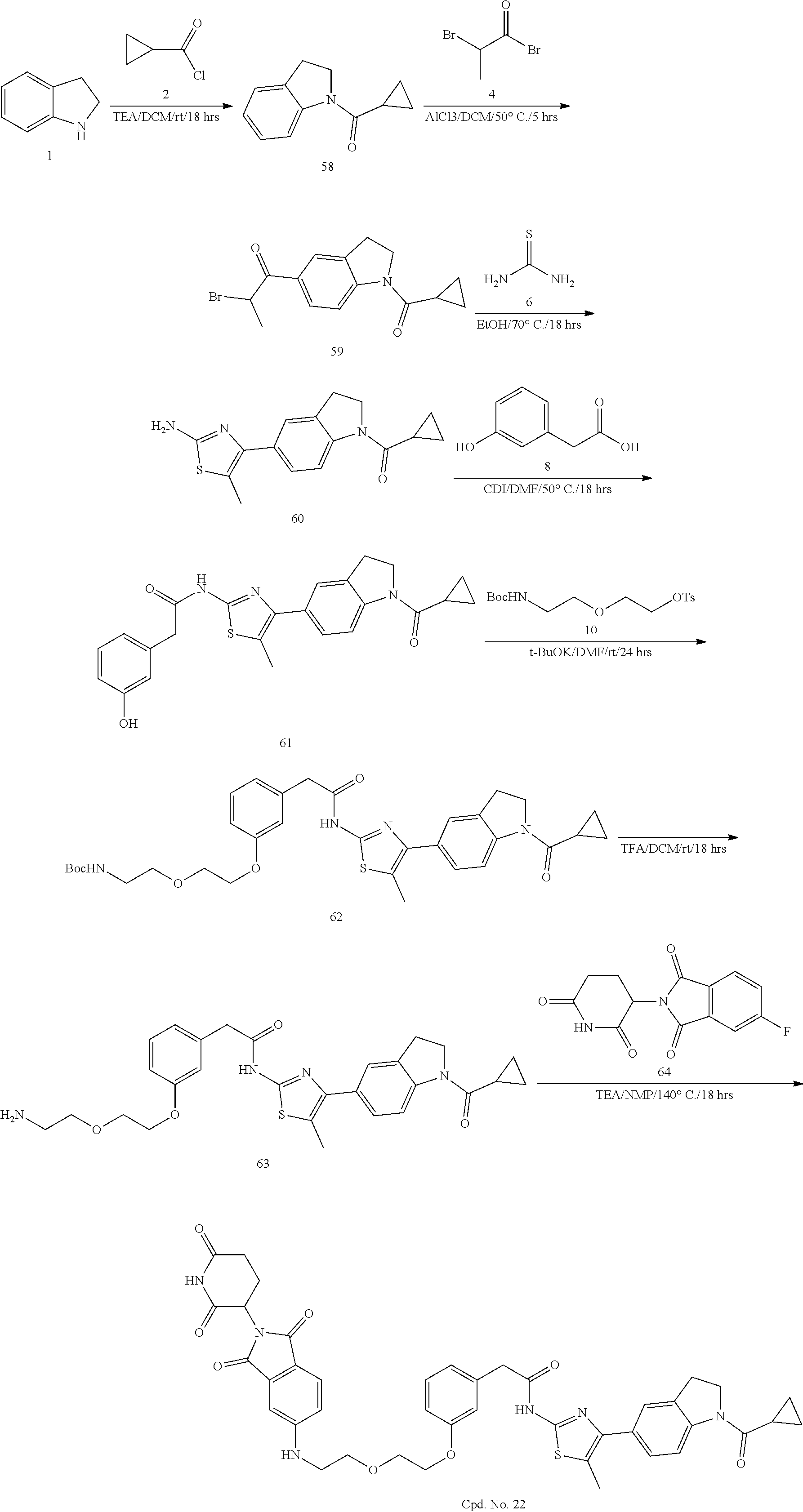

### Step 1. cyclopropyl(indolin-1-yl)methanone

To a solution of indoline (5.00 g, 42.0 mmol) and triethylamine (12.74 g, 125.9 mmol) in dichloromethane (150 mL) was added cyclopropanecarbonyl chloride (4.39 g, 42.0 mmol) at 0° C. After addition, the mixture was stirred at room temperature for 18 hrs. The mixture was quenched with water (50 mL), extracted with dichloromethane (50 mL), washed with saturated ammonium chloride aqueous solution (50 mL×2), dried over anhydrous sodium sulfate, filtered and concentrated in vacuo to give cyclopropyl(indolin-1-yl)methanone (6.0 g, 77% yield) as a white solid. MS (ESI) m/z 188.3 [M+H]⁺.

### Step 2. 2-bromo-1-(1-(cyclopropanecarbonyl)indolin-5-yl)propan-1-one

Cyclopropyl(indolin-1-yl)methanone (6.00 g, 32.0 mmol) and aluminum chloride (21.36 g, 160.2 mmol) was dissolved in dichloromethane (150 mL), then 2-bromopropanoyl bromide (34.58 g, 160.2 mmol) was added. The mixture was stirred at 50° C. for 5 hrs. The mixture was quenched with water (200 mL), basified to pH=8-10 with 6N sodium hydroxide solution, extracted with dichloromethane (600 mL), dried over anhydrous sodium sulfate, filtered and concentrated in vacuo to give crude 2-bromo-1-(1-(cyclopropanecarbonyl)indolin-5-yl)propan-1-one (10.0 g, 97% yield) as a yellow oil. MS (ESI) m/z 322.0 and 324.0 [M+H]⁺.

### Step 3. (5-(2-amino-5-methylthiazol-4-yl)indolin-1-yl)(cyclopropyl)methanone

A solution of 2-bromo-1-(1-(cyclopropanecarbonyl)indolin-5-yl)propan-1-one (10.0 g, 31.0 mmol) and thiourea (5.91 g, 77.6 mmol) in ethanol (150 mL) was stirred at 70° C. for 18 hrs. The solvent was removed in vacuo and the residue was purified by flash column chromatography (ethyl acetate/petroleum ether=1:1) to give (5-(2-amino-5-methylthiazol-4-yl)indolin-1-yl)(cyclopropyl)methanone (7.0 g, 76% yield) as a yellow solid. MS (ESI) m/z 300.1 [M+H]⁺.

### Step 4. N-(4-(1-(cyclopropanecarbonyl)indolin-5-yl)-5-methylthiazol-2-yl)-2-(3-hydroxyphenyl)acetamide

A solution of 2-(3-hydroxyphenyl)acetic acid (1.00 g, 6.57 mmol), di(1H-imidazol-1-yl)methanone (1.60 g, 9.86 mmol) and A, N-diisopropylethylamine (3.40 g, 26.3 mmol) in N,N-dimethylformamide (20 mL) was stirred at room temperature for 30 min. (5-(2-amino-5-methylthiazol-4-yl)indolin-1-yl)(cyclopropyl)methanone (2.16 g, 7.23 mmol) was added into the resulting mixture. The mixture was stirred at 65° C. for 18 hrs. The mixture was cooled to room temperature, diluted with water (100 mL), extracted with dichloromethane (50 mL×2), dried over anhydrous sodium sulfate, filtered and concentrated in vacuo. The residue was purified by flash column chromatography (dichloromethane/methanol=3/97) to N-(4-(1-(cyclopropanecarbonyl)indolin-5-yl)-5-methylthiazol-2-yl)-2-(3-hydroxyphenyl)acetamide (0.50 g, 17.6% yield) as a white solid. MS (ESI) m/z 434.1 [M+H]⁺.

### Step 5. tert-butyl (2-(2-(3-(2-((4-(1-(cyclopropanecarbonyl)indolin-5-yl)-5-methylthiazol-2-yl)amino)-2-oxoethyl)phenoxy)ethoxy)ethyl)carbamate

A solution of N-(4-(1-(cyclopropanecarbonyl)indolin-5-yl)-5-methylthiazol-2-yl)-2-(3-hydroxyphenyl)acetamide (0.50 g, 1.15 mmol), 2-(2-((tert-butoxycarbonyl)amino)ethoxy)ethyl 4-methylbenzenesulfonate (0.46 g, 1.28 mmol), potassium t-butoxide (0.39 g, 3.46 mmol) in N,N-dimethylformamide (15 mL) was stirred at room temperature for 24 hrs. The mixture was quenched with water (50 mL), extracted with dichloromethane (50 mL×2), dried over anhydrous Na₂SO₄, filtered and concentrated in vacuo. The residue was purified by flash column chromatography (dichloromethane/methane=3/97) to give crude tert-butyl (2-(2-(3-(2-((4-(1-(cyclopropanecarbonyl)indolin-5-yl)-5-methylthiazol-2-yl)amino)-2-oxoethyl)phenoxy)ethoxy)ethyl)carbamate (0.30 g, 42% yield) as a white solid. MS (ESI) m/z 621.0 [M+H]⁺.

### Step 6. 2-(3-(2-(2-aminoethoxy)ethoxy)phenyl)-N-(4-(1-(cyclopropanecarbonyl)indolin-5-yl)-5-methylthiazol-2-yl)acetamide

A solution of tert-butyl (2-(2-(3-(2-((4-(1-(cyclopropanecarbonyl)indolin-5-yl)-5-methylthiazol-2-yl)amino)-2-oxoethyl)phenoxy)ethoxy)ethyl)carbamate (0.30 g, 0.48 mmol) in trifluoracetic acid (30 mL) and dichloromethane (15 mL) was stirred at room temperature for 18 hrs. The mixture was diluted with water (100 mL), extracted with 2-methoxy-2-methylpropane (50.0×2). Then the aqueous phase was basified with saturated sodium carbonate aqueous solution, extracted dichloromethane (50 mL×2), dried over anhydrous sodium sulfate, filtered and concentrated in vacuo. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15.0 min) to give 2-(3-(2-(2-aminoethoxy)ethoxy)phenyl)-N-(4-(1-(cyclopropanecarbonyl)indolin-5-yl)-5-methylthiazol-2-yl)acetamide (20 mg, 8% yield) as a white solid. MS (ESI) m/z 521.0 [M+H]⁺.

### Step 7. N-(4-(1-(cyclopropanecarbonyl)indolin-5-yl)-5-methylthiazol-2-yl)-2-(3-(2-(2-((2-(2,6-dioxopiperidin-3-yl)-1,3-dioxoisoindolin-5-yl)amino)ethoxy)ethoxy)phenyl)acetamide

A solution of 2-(3-(2-(2-aminoethoxy)ethoxy)phenyl)-N-(4-(1-(cyclopropanecarbonyl)indolin-5-yl)-5-methylthiazol-2-yl)acetamide (0.020 g, 0.038 mmol), 2-(2,6-dioxopiperidin-3-yl)-5-fluoroisoindoline-1,3-dione (0.012 g, 0.04 mmol) and N, N-diisopropylethylamine (0.020 g, 0.15 mmol) in 1-methylpyrrolidin-2-one (1.0 mL) was stirred at 140° C. for 18 brs. The reaction mixture was purified by Prep-HPLC (Column: Welch Xtimate 21.2×250 mm C18, 10 μm, Mobile Phase: A: water (10 mmol/L ammonium bicarbonate) B: acetonitrile; B %: 30%-70% in 15.0 min) to give N-(4-(1-(cyclopropanecarbonyl)indolin-5-yl)-5-methylthiazol-2-yl)-2-(3-(2-(2-((2-(2,6-dioxopiperidin-3-yl)-1,3-dioxoisoindolin-5-yl)amino)ethoxy)ethoxy)phenyl)acetamide (4.4 mg, 15% yield) as a yellow solid. ¹H NMR (400 MHz, DMSO-d₆): δ 11.10 (s, 1H), 8.06 (m, 1H), 7.55 (d, J=8.4 Hz, 1H), 7.50 (s, 1H), 7.42 (d, J=7.6 Hz, 1H), 7.25-7.18 (m, 2H), 7.07 (s, 1H), 8.92-6.88 (m, 3H), 6.83 (d, J=8.0 Hz, 1H), 5.30 (dd, J=13.2 & 5.2 Hz, 1H), 4.39-4.28 (m, 2H), 4.10-4.09 (m, 2H), 3.78 (m, 2H), 3.70-3.65 (m, 3.39-3.38 (m, 2H), 3.23-3.21 (m, 2H), 2.91-2.71 (m, 1H), 2.67-2.57 (m, 2H), 2.43 (s, 3H), 2.00-1.97 (m, 2H), 0.88-0.86 (m, 4H). MS (ESI) m/z: 777.2 [M+H]⁺. Note: Acidic proton typically around 12.3 ppm is not visible).

### Example 21

### Synthesis of 2-(3-(2-(2-((2-(2,6-dioxopiperidin-3-yl)-1,3-dioxoisoindolin-5-yl)amino)ethoxy)ethoxy)phenyl)-N-(5-methyl-4-(1-(1-methyl-1H-imidazole-5-carbonyl)indolin-5-yl)thiazol-2-yl)acetamide (Cpd. No. 23)

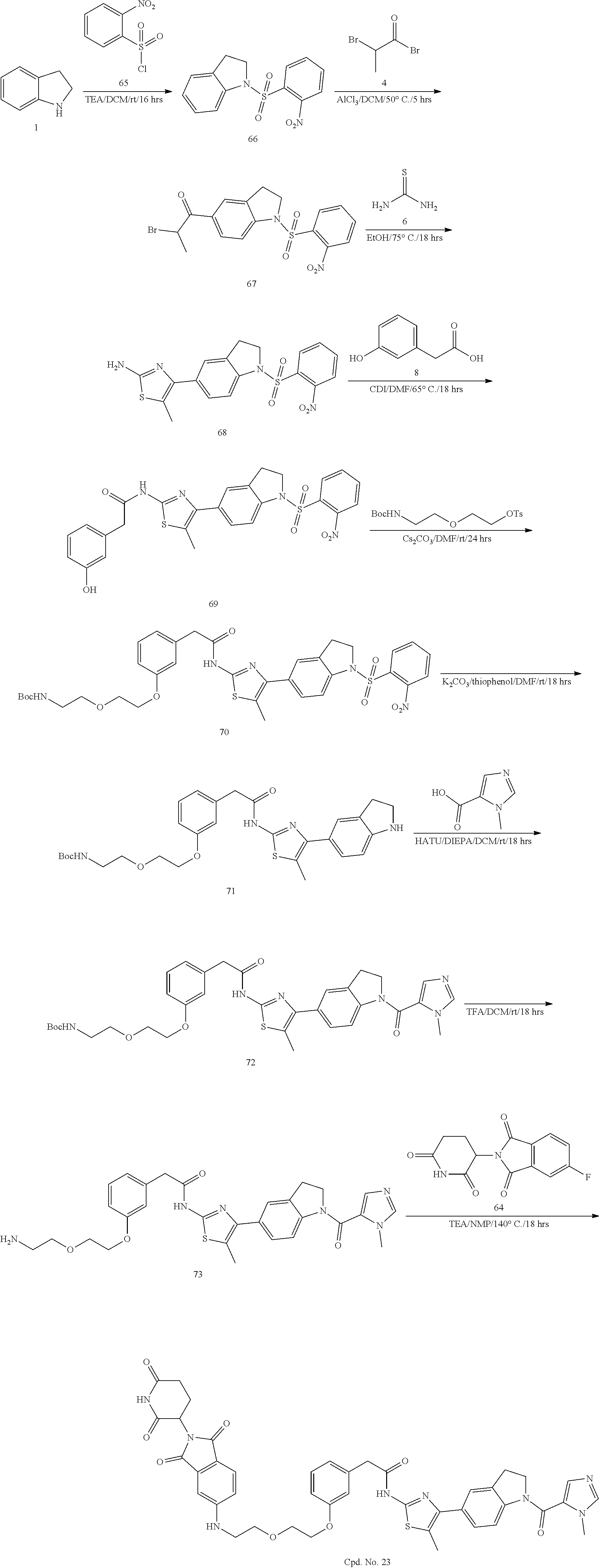

### Step 1. 1-((2-nitrophenyl)sulfonyl)indoline

To a solution of indoline (5.00 g, 42.0 mmol) and triethylamine (12.74 g, 125.9 mmol) in dichloromethane (150 mL), was added 2-nitrobenzenesulfonyl chloride (9.29 g, 42.0 mmol) at 0° C. After addition, the mixture was stirred at room temperature for 16 hrs. The reaction mixture was diluted with water (50 mL), extracted with dichloromethane (50 mL), washed with brine (50 mL×2), dried over anhydrous sodium sulfate, filtered and concentrated in vacuo to give 1-((2-nitrophenyl)sulfonyl)indoline (7.0 g, 55% yield) as a yellow solid. MS (ESI) m/z: 305.0 [M+H]⁺.

### Step 2. 2-bromo-1-(1-((2-nitrophenyl)sulfonyl)indolin-5-yl)propan-1-one

1-((2-nitrophenyl)sulfonyl)indoline (7.0 g, 23.0 mmol) and aluminum chloride (9.21 g, 69.1 mmol) was dissolved in dichloromethane (150 mL), then 2-bromopropanoyl bromide (14.91 g, 69.07 mmol) was added. The mixture was stirred at 50° C. for 5 hrs. The mixture was quenched with water (500 mL), basified to pH=8-10 with 6N NaOH solution, extracted with dichloromethane (100 mL), dried over anhydrous sodium sulfate, filtered and concentrated in vacuo to give crude 2-bromo-1-(1-((2-nitrophenyl)sulfonyl)indolin-5-yl)propan-1-one (9.0 g, 89%) as a yellow oil. MS (ESI) m/z: 439.0 and 441.0 [M+H]⁺.

### Step 3. 5-methyl-4-(1-((2-nitrophenyl)sulfonyl)indolin-5-yl)thiazol-2-amine

A solution of 2-bromo-1-(1-((2-nitrophenyl)sulfonyl)indolin-5-yl)propan-1-one (9.0 g, 20.5 mmol) and thiourea (3.89 g, 51.2 mmol) in ethanol (150 mL) was stirred at 75° C. for 18 hrs. The mixture was cooled to room temperature, diluted with water (100 mL), extracted with dichloromethane (200 mL), washed with brine (50 mL×3), dried over anhydrous sodium sulfate, filtered and concentrated in vacuo. The residue was purified by flash column chromatography (ethyl acetate/petroleum ether=1:1) to give 5-methyl-4-(1-((2-nitrophenyl)sulfonyl)indolin-5-yl)thiazol-2-amine (6.0 g, 72% yield) as a yellow solid. MS (ESI) m/z: 417.1 [M+H]⁺.

### Step 4. 2-(3-hydroxyphenyl)-N-(5-methyl-4-(1-((2-nitrophenyl)sulfonyl)indolin-5-yl)thiazol-2-yl)acetamide

A solution of 2-(3-hydroxyphenyl)acetic acid (2.00 g, 13.1 mmol), di(1H-imidazol-1-yl)methanone (3.19 g, 19.7 mmol) and A, N-diisopropylethylamine (6.79 g, 52.6 mmol) in N,N-dimethylformamide (40 mL) was stirred at room temperature for 30 min. 5-methyl-4-(1-((2-nitrophenyl)sulfonyl)indolin-5-yl)thiazol-2-amine (6.02 g, 14.5 mmol) was added into the resulting mixture. The mixture was stirred at 65° C. for 18 hrs. The mixture was cooled to room temperature, diluted with water (100 mL), extracted with dichloromethane (50 mL×2), dried over anhydrous sodium sulfate, filtered and concentrated in vacuo. The residue was purified by flash column chromatography (dichloromethane/methanol=3/97) to give 2-(3-hydroxyphenyl)-N-(5-methyl-4-(1-((2-nitrophenyl)sulfonyl)indolin-5-yl)thiazol-2-yl)acetamide (1.8 g, 25% yield) as a white solid. MS (ESI) m/z: 551.0 [M+H]⁺.

### Step 5. tert-butyl (2-(2-(3-(2-((5-methyl-4-(1-((2-nitrophenyl)sulfonyl)indolin-5-yl)thiazol-2-yl)amino)-2-oxoethyl)phenoxy)ethoxy)ethyl)carbamate

A solution of 2-(3-hydroxyphenyl)-N-(5-methyl-4-(1-((2-nitrophenyl)sulfonyl)indolin-5-yl)thiazol-2-yl)acetamide (1.50, 2.72 mmol), 2-(2-((tert-butoxycarbonyl)amino)ethoxy)ethyl 4-methylbenzenesulfonate (1.37 g, 3.81 mmol), cesium carbonate (1.79 g, 5.45 mmol) in N,N-dimethylformamide (30 mL) was stirred at room temperature for 24 hrs. The mixture was diluted with water (50 mL), extracted with dichloromethane (50 mL×2), dried over anhydrous sodium sulfate, filtered and concentrated in vacuo. The residue was purified by flash column chromatography (dichloromethane/methanol=3/97) to give tert-butyl (2-(2-(3-(2-((5-methyl-4-(1-((2-nitrophenyl)sulfonyl)indolin-5-yl)thiazol-2-yl)amino)-2-oxoethyl)phenoxy)ethoxy)ethyl)carbamate (1.5 g, 75% yield) as a white solid. MS (ESI) m/z: 738.2 [M+H]⁺.

### Step 6. tert-butyl (2-(2-(3-(2-((4-(indolin-5-yl)-5-methylthiazol-2-yl)amino)-2-oxoethyl)phenoxy)ethoxy)ethyl)carbamate

A solution of tert-butyl (2-(2-(3-(2-((5-methyl-4-(1-((2-nitrophenyl)sulfonyl)indolin-5-yl)thiazol-2-yl)amino)-2-oxoethyl)phenoxy)ethoxy)ethyl)carbamate (1.40 g, 1.90 mmol), potassium carbonate (0.29 g, 2.09 mmol), thiophenol (0.42 g, 3.8 mmol) in N,N-dimethylformamide (20.0 mL) was stirred at room temperature for 18 hrs. The reaction mixture was diluted with water (40 mL), extracted with dichloromethane (50 mL×2), dried over anhydrous sodium sulfate, filtered and concentrated in vacuo. The residue was purified by flash column chromatography (dichloromethane/methanol=20/1) to give tert-butyl (2-(2-(3-(2-((4-(indolin-5-yl)-5-methylthiazol-2-yl)amino)-2-oxoethyl)phenoxy)ethoxy)ethyl)carbamate (0.8 g, 76% yield) as a white solid. MS (ESI) m/z: 553.2 [M+H]⁺.

### Step 7. tert-butyl (2-(2-(3-(2-((5-methyl-4-(1-(1-methyl-1H-imidazole-5-carbonyl)indolin-5-yl)thiazol-2-yl)amino)-2-oxoethyl)phenoxy)ethoxy)ethyl)carbamate

A solution of 1-methyl-1H-imidazole-5-carboxylic acid (0.05 g, 0.40 mmol), tert-butyl (2-(2-(3-(2-((4-(indolin-5-yl)-5-methylthiazol-2-yl)amino)-2-oxoethyl)phenoxy)ethoxy)ethyl)carbamate (0.26 g, 0.48 mmol), 2-(7-azabenzotriazol-1-yl)-N,N,N′,N′-tetramethyluronium hexafluorophosphate (0.23 g, 0.59 mmol) and triethylamine (0.14 g, 1.4 mmol) in dichloromethane (20 mL) was stirred at room temperature for 18 hrs. The mixture was diluted with water (20 mL), extracted with dichloromethane (50 mL), dried over anhydrous sodium sulfate, filtered and concentrated in vacuo. The residue was purified by flash column chromatography (dichloromethane/methanol=3/97) to give tert-butyl (2-(2-(3-(2-((5-methyl-4-(1-(1-methyl-1H-imidazole-5-carbonyl)indolin-5-yl)thiazol-2-yl)amino)-2-oxoethyl)phenoxy)ethoxy)ethyl)carbamate (0.12 g, 46%) as a white solid. MS (ESI) m/z: 661.0 [M+H]⁺.

### Step 8. 2-(3-(2-(2-aminoethoxy)ethoxy)pbenyl)-N-(5-methyl-4-(1-(1-methyl-1H-imidazole-5-carbonyl)indolin-5-yl)thiazol-2-yl)acetamide

A solution of tert-butyl (2-(2-(3-(2-((5-methyl-4-(1-(1-methyl-1H-imidazole-5-carbonyl)indolin-5-yl)thiazol-2-yl)amino)-2-oxoethyl)phenoxy)ethoxy)ethyl)carbamate (0.12 g, 0.18 mmol) in trifluoracetic acid (15 mL) and dichloromethane (15 mL) was stirred at room temperature for 18 hrs. The solvent was removed in vacuo and the residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15.0 min) to give 2-(3-(2-(2-aminoethoxy)ethoxy)phenyl)-N-(5-methyl-4-(1-(1-methyl-1H-imidazole-5-carbonyl)indolin-5-yl)thiazol-2-yl)acetamide (65 mg, 65% yield) as a white solid. MS (ESI) m/z: 561.0 [M+H]⁺.

### Step 9. 2-(3-(2-(2-((2-(2,6-dioxopiperidin-3-yl)-1,3-dioxoisoindolin-5-yl)amino)ethoxy)ethoxy)phenyl)-N-(5-methyl-4-(1-(1-methyl-1H-imidazole-5-carbonyl)indolin-5-yl)thiazol-2-yl)acetamide

A solution of 2-(3-(2-(2-aminoethoxy)ethoxy)phenyl)-N-(5-methyl-4-(1-(1-methyl-1H-imidazole-5-carbonyl)indolin-5-yl)thiazol-2-yl)acetamide (0.065 g, 0.12 mmol), 2-(2,6-dioxopiperidin-3-yl)-5-fluoroisoindoline-1,3-dione (0.036 g, 0.13 mmol) and N,N-diisopropylethylamine (0.061 g, 0.47 mmol) in 1-methylpyrrolidin-2-one (2.0 mL) was stirred at 140° C. for 18 hrs. The reaction mixture was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15.0 min) to give 2-(3-(2-(2-((2-(2,6-dioxopiperidin-3-yl)-1,3-dioxoisoindolin-5-yl)amino)ethoxy)ethoxy)phenyl)-N-(5-methyl-4-(1-(1-methyl-1H-imidazole-5-carbonyl)indolin-5-yl)thiazol-2-yl)acetamide (6.7 mg, 7% yield) as a yellow solid. ¹H NMR (400 MHz, DMSO-d₆): δ 8.07 & 5.30* (d, J=8.0 Hz, 1H), 7.85 (s, 1H), 7.59-7.54 (m, 3H), 7.49 (d, J=4.8 Hz, 1H), 7.25-7.17 (m, 2H), 7.00 (s, 1H), 6.92-6.88 (m, 3H), 6.83 (d, J=8.0 Hz, 1H), 5.30 (dd, J=13 & 5.4 Hz, 1H), 4.36 (t, J=8.2 Hz, 2H), 4.10 (t, J=4.0 Hz, 2H), 3.80-3.75 (m, 5H), 3.70-3.64 (m, 3.43-3.38 (m, 2H), 3.19 (t, J=8.4 Hz, 2H), 2.91-2.80 (m, 1H), 2.79-2.67 (m, 2H), 2.45 (s, 3H), 2.00-1.97 (m, 1H). MS (ESI) m/z: 817.2 [M+H]⁺. (Note: 2 acidic protons that are typically around 12.3 ppm and 11.1 ppm are not visible). *Multiple signals arising from conformational isomers.

### Example 22

### Synthesis of 2-(3-((20-((2-(2,6-dioxopiperidin-3-yl)-1,3-dioxoisoindolin-4-yl)oxy)-3,6,9,12,15,18-hexaoxaicosyl)oxy)phenyl)-N-(5-methyl-4-(1-(2-methylbenzoyl)indolin-5-yl)thiazol-2-yl)acetamide (Cpd. No. 11)

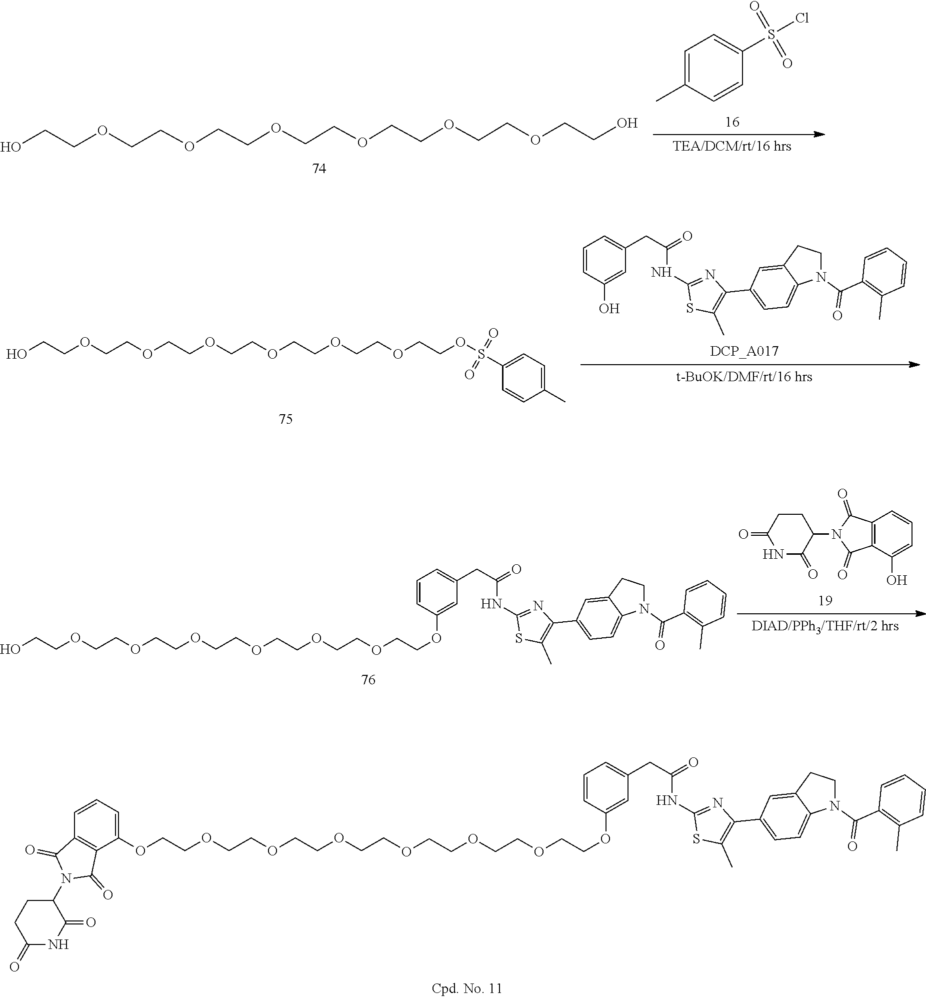

### Step 1. 20-hydroxy-3,6,9,12,15,18-hexaoxaicosyl 4-methylbenzenesulfonate

A solution of 3,6,9,12,15,18-hexaoxaicosane-1,20-diol (5.0 g, 15.3 mmol), triethylamine (4.6 g, 45.9 mmol) and 4-methylbenzenesulfonyl chloride (1.4 g, 7.6 mmol) in dichloromethane (50 mL) was stirred at room temperature for 16 hrs. The mixture was quenched with water (50 mL), extracted with ethyl acetate (80 mL×2), dried over sodium sulfate and concentrated in vacuo. The residue was purified by flash column chromatography (ethyl acetate/petroleum ether=1:1) to give 20-hydroxy-3,6,9,12,15,18-hexaoxaicosyl 4-methylbenzenesulfonate (1.2 g, 32.6% yield) as a yellow oil. MS (ESI) m/z 481.3 [M+H]⁺.

### Step 2. 2-(3-((20-hydroxy-3,6,9,12,15,18-hexaoxaicosyl)oxy)phenyl)-N-(5-methyl-4-(1-(2-methylbenzoyl)indolin-5-yl)thiazol-2-yl)acetamide

A mixture of 20-hydroxy-3,6,9,12,15,18-hexaoxaicosyl 4-methylbenzenesulfonate (0.30 g, 0.62 mmol), 2-(3-hydroxyphenyl)-N-(5-methyl-4-(1-(2-methylbenzoyl)indolin-5-yl)thiazol-2-yl)acetamide (0.36 g, 0.75 mmol), potassium t-butoxide (0.21 g, 1.87 mmol) in N,N-dimethylformamide (10 mL) was stirred at room temperature for 16 hrs. The reaction mixture was quenched with water (30 mL), extracted with ethyl acetate (30 mL×2), dried over sodium sulfate, filtered and concentrated in vacuo. The residue was purified by flash column chromatography (ethyl acetate/petroleum ether=1:1) to give 2-(3-((20-hydroxy-3,6,9,12,15,18-hexaoxaicosyl)oxy)phenyl)-N-(5-methyl-4-(1-(2-methylbenzoyl)indolin-5-yl)thiazol-2-yl)acetamide (0.26 g, 52.6% yield) as a white solid. MS (ESI) m/z 792.4 [M+H]⁺.

### Step 3. 2-(3-((20-((2-(2,6-dioxopiperidin-3-yl)-1,3-dioxoisoindolin-4-yl)oxy)-3,6,9,12,15,18-hexaoxaicosyl)oxy)phenyl)-N-(5-methyl-4-(1-(2-methylbenzoyl)indolin-5-yl)thiazol-2-yl)acetamide

To a solution of 2-(3-((20-hydroxy-3,6,9,12,15,18-hexaoxaicosyl)oxy)phenyl)-N-(5-methyl-4-(1-(2-methylbenzoyl)indolin-5-yl)thiazol-2-yl)acetamide (0.26 g, 0.33 mmol), 2-(2,6-dioxopiperidin-3-yl)-4-hydroxyisoindoline-1,3-dione (0.10 g, 0.39 mmol) and triphenylphosphine (0.17 g, 0.66 mmol) in tetrahydrofuran (6.0 mL), was added diethyl azodicarboxylate (0.07 g, 0.39 mmol) at room temperature. The mixture was stirred at room temperature for 2 hrs. The mixture was diluted with water (20 mL), extracted with dichloromethane (20 mL×2), dried over anhydrous sodium sulfate, filtered and concentrated in vacuo. The residue was purified by Prep-HPLC (Column: Welch Xtimate 21.2×250 mm C18, 10 μm, Mobile Phase: A: water (10 mmol/L ammonium bicarbonate) B: acetonitrile; B %: 30%-70% in 15.0 min) to give 2-(3-((20-((2-(2,6-dioxopiperidin-3-yl)-1,3-dioxoisoindolin-4-yl)oxy)-3,6,9,12,15,18-hexaoxaicosyl)oxy)phenyl)-N-(5-methyl-4-(1-(2-methylbenzoyl)indolin-5-yl)thiazol-2-yl)acetamide (52 mg, 15%) as white solid. ¹H NMR (400 MHz, DMSO-d₆): δ12.33 & 12.22 (s, 1H)*, 11.11 (s, 1H), 8.23 & 5.45* (d, J=8.4 Hz, 1H), 7.79 (t, J=7.8 Hz, 1H), 7.52-7.50 (m, 2H), 7.52 &7.03* (m, 1H), 7.45 (d, J=7.2 Hz, 1H), 7.37-7.30 (m, 4H), 7.23 (t, J=8.0 Hz, 1H), 6.92-6.89 (m, 2H), 6.84 (d, J=8.0 Hz, 1H), 5.08 (dd, J=12.8 & 5.6 Hz, 1H), 4.33 (t, J=4.2 Hz, 2H), 4.21 & 3.74 (m, 2H)*, 4.06 (t, J=4.0 Hz, 2H), 3.79 (t, J=4.3 Hz, 2H), 3.70-3.63 (m, 3.64-3.61 (m, 2H), 3.58-3.56 (m, 2H), 3.53-3.47 (m, 161), 3.13 (t, J=8.2 Hz, 2H), 2.89-2.83 (m, 1-1), 2.60-2.55 (m, 2H), 2.48 & 2.33 (s, 3H)*, 2.29 & 2.17 (s, 3H)*, 2.04-1.00 (m, 1H). MS (ESI) m/z 1048.4 [M+H]⁺. *Multiple signals arising from conformational isomers.

### Example 23

### Synthesis of 2-(3-((20-((2-(2,6-dioxopiperidin-3-yl)-1,3-dioxoisoindolin-5-yl)oxy)-3,6,9,12,15,18-hexaoxaicosyl)oxy)phenyl)-N-(5-methyl-4-(1-(2-methylbenzoyl)indolin-5-yl)thiazol-2-yl)acetamide (Cpd. No. 16)

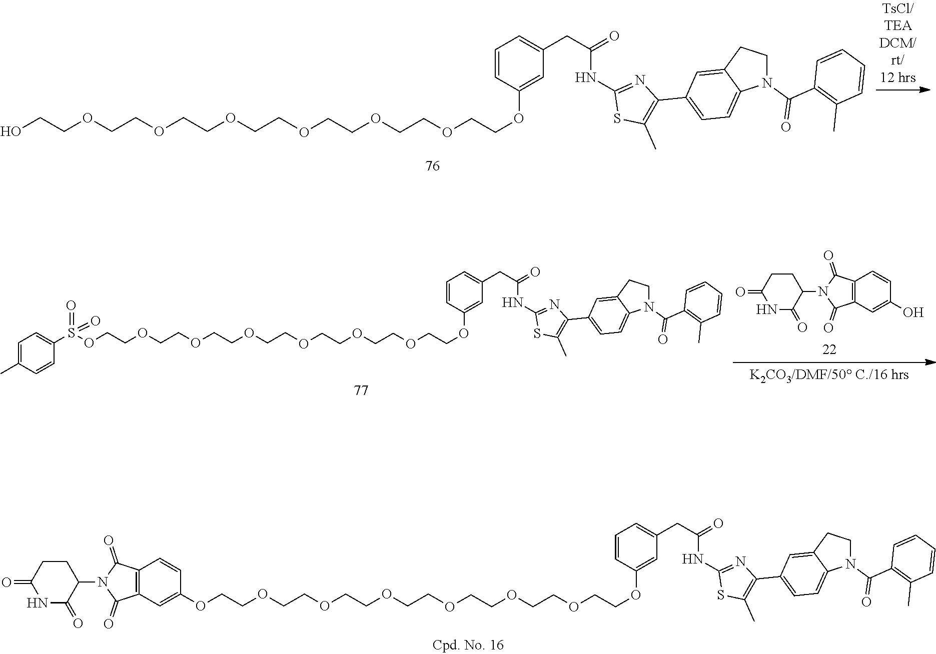

### Step 1. 20-(3-(2-((5-methyl-4-(1-(2-methylbenzoyl)indolin-5-yl)thiazol-2-yl)amino)-2-oxoethyl)phenoxy)-3,6,9,12,15,18-hexaoxaicosyl 4-methylbenzenesulfonate

A solution of 2-(3-((20-hydroxy-3,6,9,12,15,18-hexaoxaicosyl)oxy)phenyl)-N-(5-methyl-4-(1-(2-methylbenzoyl)indolin-5-yl)thiazol-2-yl)acetamide (0.24 g, 0.30 mmol), tosyl chloride (0.06 g, 0.30 mmol) and triethylamine (0.091 g, 0.90 mmol) in dichloromethane (10 mL) was stirred at room temperature for 12 hrs. The mixture was diluted with water (10 mL), extracted with dichloromethane (30 mL), dried over anhydrous sodium sulfate, filtered and concentrated in vacuo. The residue was purified by flash column chromatography (dichloromethane) to give 20-(3-(2-((5-methyl-4-(1-(2-methylbenzoyl)indolin-5-yl)thiazol-2-yl)amino)-2-oxoethyl)phenoxy)-3,6,9,12,15,18-hexaoxaicosyl 4-methylbenzenesulfonate (0.13 g, 45.4% yield) as a white solid. MS (ESI) m/z 946.4 [M+H]⁺.

### Step 2. 2-(3-((20-((2-(2,6-dioxopiperidin-3-yl)-1,3-dioxoisoindolin-5-yl)oxy)-3,6,9,12,15,18-hexaoxaicosyl)oxy)phenyl)-N-(5-methyl-4-(1-(2-methylbenzoyl)indolin-5-yl)thiazol-2-yl)acetamide

A solution of 20-(3-(2-((5-methyl-4-(1-(2-methylbenzoyl)indolin-5-yl)thiazol-2-yl)amino)-2-oxoethyl)phenoxy)-3,6,9,12,15,18-hexaoxaicosyl 4-methylbenzenesulfonate (0.11 g, 0.12 mmol), 2-(2,6-dioxopiperidin-3-yl)-5-hydroxyisoindoline-1,3-dione (0.03 g, 0.12 mmol) and potassium carbonate (0.07 g, 0.53 mmol) in N,N-dimethylformamide (8.0 mL) was stirred at 50° C. for 16 hrs. The reaction mixture was purified by Prep-HPLC (Column: Welch Xtimate 21.2×250 mm C18, 10 μm, Mobile Phase: A: water (10 mmol/L ammonium bicarbonate) B: acetonitrile; B %: 30%-70% in 15.0 min) to give 2-(3-((20-((2-(2,6-dioxopiperidin-3-yl)-1,3-dioxoisoindolin-5-yl)oxy)-3,6,9,12,15,18-hexaoxaicosyl)oxy)phenyl)-N-(5-methyl-4-(1-(2-methylbenzoyl)indolin-5-yl)thiazol-2-yl)acetamide (9.0 mg, 7.3%) as a white solid. ¹H NMR (400 MHz, DMSO-d₆): δ12.33 & 12.22 (s, 1H)*, 11.11 (s, 1H), 8.24 & 5.45* (d, J=8.4 Hz, 1H), 7.82 (d, J=8.4 Hz, 1H), 7.53 (brs, 1H), 7.53 & 7.01* (brs, 1H), 7.45 (d, J=2.0 Hz, 1H), 7.37-7.30 (m, 5H), 7.24 (t, J=7.8 Hz, 1H), 6.92-6.89 (m, 2H), 6.84 (d, J=8.4 Hz, 1H), 5.12 (dd, J=13.0 & 5.4 Hz, 1H), 4.30 (t, J=4.4 Hz, 2H), 4.21 & 3.69 (m, 2H)*, 4.07 (m, 2H), 3.78-3.71 (m, 6H), 3.59-3.47 (m, 3.54-3.46 (m, 16H), 3.13 (t, J=7.8 Hz, 2H), 2.88-2.85 (m, 1H), 2.62-2.55 (m, 2H), 2.46 & 2.33 (s, 3H)*, 2.30 & 2.18 (s, 3H)*, 2.05-2.00 (m, 1H). MS (ESI) m/z 525 [M/2+H]⁺. *Multiple signals arising from conformational isomers.

### Example 24

### Synthesis of 2-(3-((23-((2-(2,6-dioxopiperidin-3-yl)-1,3-dioxoisoindolin-5-yl)oxy)-3,6,9,12,15,18,21-heptaoxatricosyl)oxy)phenyl)-N-(5-methyl-4-(1-(2-methylbenzoyl)indolin-5-yl)thiazol-2-yl)acetamide (Cpd. No. 17)

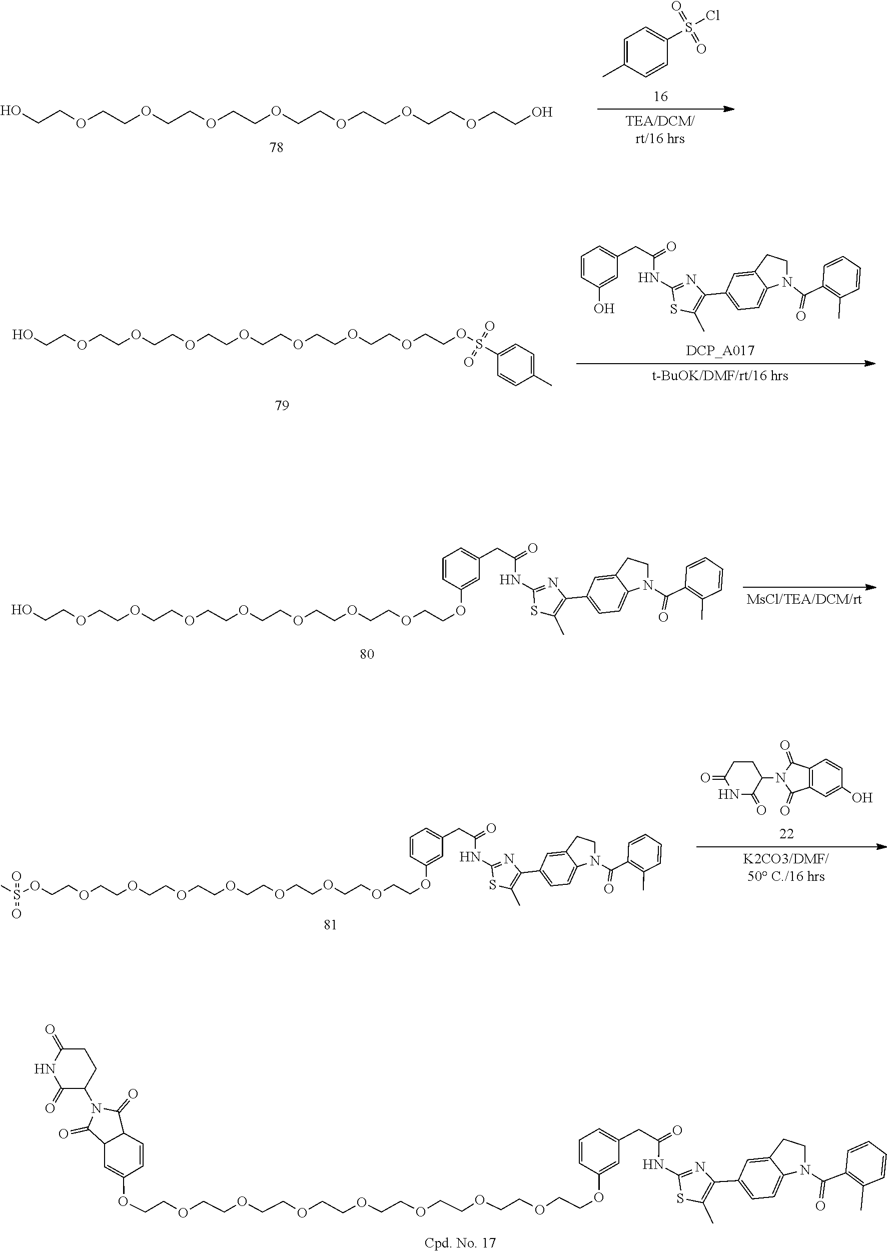

### Step 1. 23-hydroxy-3,6,9,12,15,18,21-heptaoxatricosyl 4-methylbenzenesulfonate

A solution of 3,6,9,12,15,18,21-heptaoxatricosane-1,23-diol (3.7 g, 10.0 mmol), triethylamine (3.0 g, 30 mmol) and 4-methylbenzenesulfonyl chloride (0.95 g, 5.0 mmol) in dichloromethane (30 mL) was stirred room temperature for 16 hrs. The mixture was quenched with water (50 mL), extracted with ethyl acetate (50 mL×2), dried over sodium sulfate, concentrated in vacuo. The residue was purified by flash column chromatography (ethyl acetate/petroleum ether=1:1) to give 23-hydroxy-3,6,9,12,15,18,21-heptaoxatricosyl 4-methylbenzenesulfonate (2.6 g, 99% yield) as a yellow oil. MS (ESI) m/z 525.3 [M+H]⁺.

### Step 2. 2-(3-((23-hydroxy-3,6,9,12,15,18,21-heptaoxatricosyl)oxy)phenyl)-N-(5-methyl-4-(1-(2-methylbenzoyl)indolin-5-yl)thiazol-2-yl)acetamide

A solution of 23-hydroxy-3,6,9,12,15,18,21-heptaoxatricosyl 4-methylbenzenesulfonate (0.26 g, 0.50 mmol), 2-(3-hydroxyphenyl)-N-(5-methyl-4-(1-(2-methylbenzoyl)indolin-5-yl)thiazol-2-yl)acetamide (0.24 g, 0.50 mmol), potassium t-butoxide (0.22 g, 2.0 mmol) in N,N-dimethylformamide (10 mL) was stirred at room temperature for 16 hrs. The reaction mixture was quenched with water (20 mL) and extracted with ethyl acetate (30 mL×2), dried over sodium sulfate, filtered and concentrated in vacuo. The residue was purified by flash column chromatography (ethyl acetate/petroleum ether=1:1) to give 2-(3-((23-hydroxy-3,6,9,12,15,18,21-heptaoxatricosyl)oxy)pbenyl)-N-(5-methyl-4-(1-(2-methylbenzoyl)indolin-5-yl)thiazol-2-yl)acetamide (0.30 g, 72.4% yield) as white solid. MS (ESI) m/z 837.5 [M+18]⁺

### Step 3. 23-(3-(2-((5-methyl-4-(1-(2-methylbenzoyl)indolin-5-yl)thiazol-2-yl)amino)-2-oxoethyl)phenoxy)-3,6,9,12,15,18,21-heptaoxatricosyl methanesulfonate

To a solution of 2-(3-((23-hydroxy-3,6,9,12,15,18,21-heptaoxatricosyl)oxy)phenyl)-N-(5-methyl-4-(1-(2-methylbenzoyl)indolin-5-yl)thiazol-2-yl)acetamide (0.25 g, 0.30 mmol) and triethylamine (0.91 g, 0.90 mmol) in dichloromethane (15 mL), was added methanesulfonyl chloride (0.069 g, 0.60 mmol) at 0° C. After addition, the reaction mixture was stirred room temperature for 16 hrs. The mixture was quenched with water (10 ml), extracted with ethyl acetate (20 mL×2), dried over sodium sulfate, concentrated in vacuo. The residue was purified by flash column chromatography (ethyl acetate/petroleum ether=2:3) to give 23-(3-(2-((5-methyl-4-(1-(2-methylbenzoyl)indolin-5-yl)thiazol-2-yl)amino)-2-oxoethyl)phenoxy)-3,6,9,12,15,18,21-heptaoxatricosyl methanesulfonate (0.19 g, 69.5% yield) as a yellow solid. IS (ESI) m/z 914.5 [M+H]⁺.

### Step 4. 2-(3-((23-((2-(2,6-dioxopiperidin-3-yl)-1,3-dioxoisoindolin-5-yl)oxy)-3,6,9,12,15,18,21-heptaoxatricosyl)oxy)phenyl)-N-(5-methyl-4-(1-(2-methylbenzoyl)indolin-5-yl)thiazol-2-yl)acetamide

A solution of 23-(3-(2-((5-methyl-4-(1-(2-methylbenzoyl)indolin-5-yl)thiazol-2-yl)amino)-2-oxoethyl)phenoxy)-3,6,9,12,15,18,21-heptaoxatricosyl methanesulfonate (0.19 g, 0.21 mmol) and 2-(2,6-dioxopiperidin-3-yl)-5-hydroxyisoindoline-1,3-dione (0.057 g, 0.21 mmol), potassium carbonate (0.072 g, 0.52 mmol) in N,N-dimethylformamide (8.0 mL) was stirred at 50° C. for 16 hrs. The reaction mixture was purified by Prep-HPLC (Column: Welch Xtimate 21.2×250 mm C18, 10 μm, Mobile Phase: A: water (10 mmol/L ammonium bicarbonate) B: acetonitrile; B %: 30%-70% in 15.0 min) to give 2-(3-((23-((2-(2,6-dioxopiperidin-3-yl)-1,3-dioxoisoindolin-5-yl)oxy)-3,6,9,12,15,18,21-heptaoxatricosyl)oxy)phenyl)-N-(5-methyl-4-(1-(2-methylbenzoyl)indolin-5-yl)thiazol-2-yl)acetamide (15.2 mg, 6.7% yield) as a white solid. ¹H NMR (400 MHz, 60° C., DMSO-d₆): δ12.10 (brs, 1H)*, 10.9 (s, 1H), 7.82 (d, J=8.0 Hz, 1H), 7.53 (s, 1H), 7.43 (d, J=2.0 Hz, 1H), 7.40-7.32 (m, 7H), 7.23 (t, J=8.0 Hz, 1H), 6.94-6.86 (m, 2H), 6.85 (d, J=8.0 Hz, 1H), 5.09 (dd, J=13.0 & 5.4 Hz, 1H), 4.32 (t, J=4.6 Hz, 2H), 4.21 & 3.74 (m, 2H)*, 4.10 (t, J=4.6 Hz, 2H), 3.80 (t, J=4.6 Hz, 2H), 3.77-3.72 (m, 3.62-3.60 (m, 3.56-3.51 (m, 20H), 3.13** (t, J=8.4 Hz, 2H), 2.88-2.85 (m, 1H), 2.62-2.55 (m, 2H), 2.44 (s, 3H), 2.28 (s, 3H), 2.08-2.01 (m, 1H). MS (ESI) m/z 1092.4 [M+H]⁺. *Multiple signals arising from conformational isomers. *Signal is under the water peak but is clearly visible in NMR taken at RT.

### Example 25

### Synthesis of 2-(3-(2-(4-(2-(2,6-dioxopiperidin-3-yl)-1,3-dioxoisoindolin-5-yl)piperazin-1-yl)ethoxy)phenyl)-N-(5-methyl-4-(1-(2-methylbenzoyl)indolin-5-yl)thiazol-2-yl)acetamide (Cpd. No. 24)

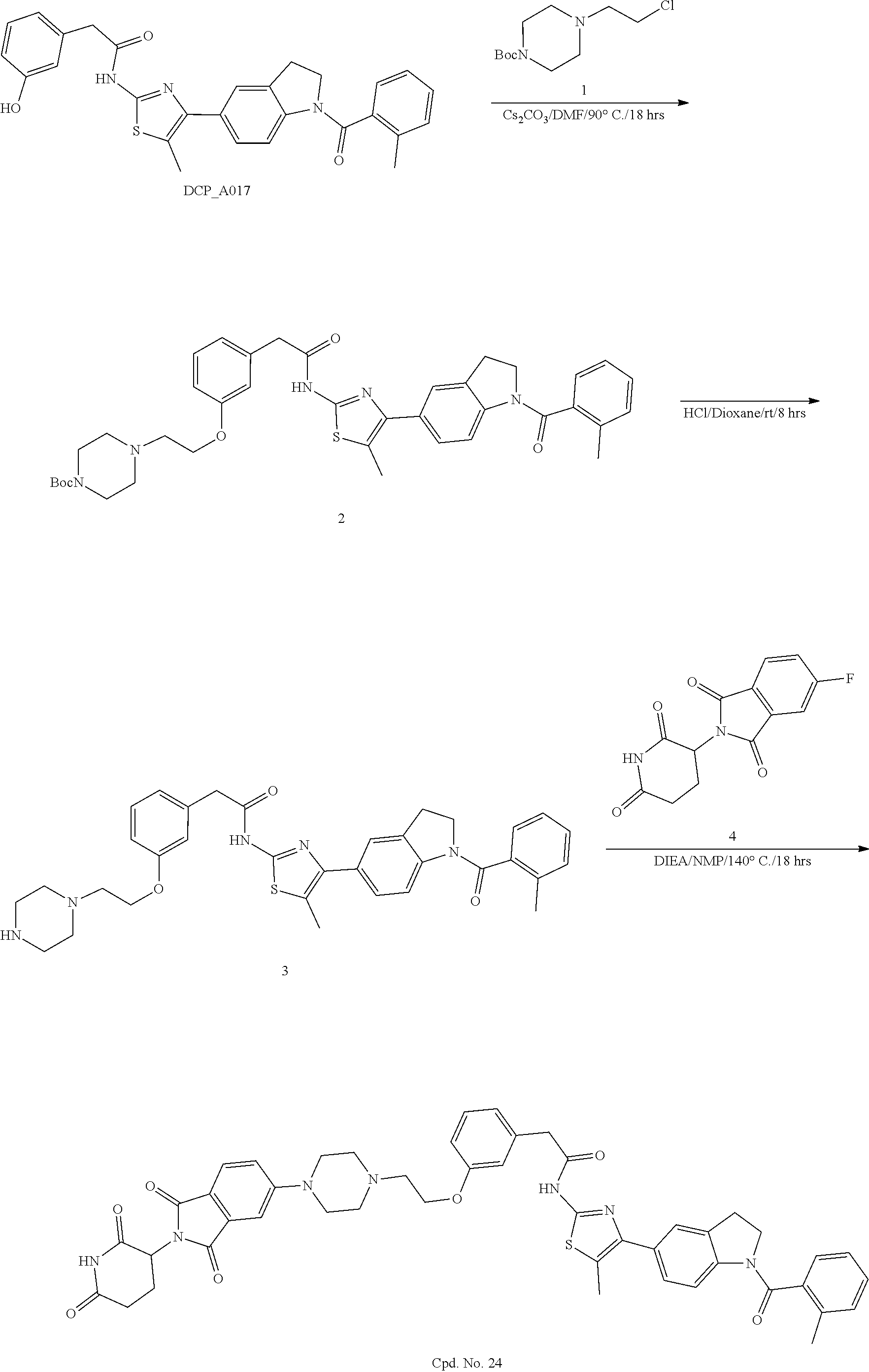 [SMILES: Cc1ccccc1C(=O)N1CCc2cc(-c3nc(NC(=O)Cc4cccc(O)c4)sc3C)ccc21 ; CC(C)(C)OC(=O)N1CCN(CCCl)CC1 ; Cc1ccccc1C(=O)N1CCc2cc(-c3nc(NC(=O)Cc4cccc(OCCN5CCN(C(=O)OC(C)(C)C)CC5)c4)sc3C)ccc21 ; Cc1ccccc1C(=O)N1CCc2cc(-c3nc(NC(=O)Cc4cccc(OCCN5CCNCC5)c4)sc3C)ccc21 ; O=C1CCC(N2C(=O)c3ccc(F)cc3C2=O)C(=O)N1 ; Cc1ccccc1C(=O)N1CCc2cc(-c3nc(NC(=O)Cc4cccc(OCCN5CCN(c6ccc7c(c6)C(=O)N(C6CCC(=O)NC6=O)C7=O)CC5)c4)sc3C)ccc21]

### Step 1. tert-butyl 4-(2-(3-(2-((5-methyl-4-(1-(2-methylbenzoyl)indolin-5-yl)thiazol-2-yl)amino)-2-oxoethyl)phenoxy)ethyl)piperazine-1-carboxylate

A solution of 2-(3-hydroxyphenyl)-N-(5-methyl-4-(1-(2-methylbenzoyl)indolin-5-yl)thiazol-2-yl) acetamide (0.50 g, 1.03 mmol), tert-butyl 4-(2-chloroethyl)piperazine-1-carboxylate (0.26 g, 1.03 mmol) and cesium carbonate (1.01 g, 3.10 mmol) in N,N-dimethylformamide (15 mL) was stirred at 90° C. for 18 hrs. The mixture was cooled to room temperature, quenched with water (10 mL), extracted with dichloromethane (50 mL), washed with brine (20 mL), dried over anhydrous sodium sulfate, filtered and concentrated in vacuo. The residue was purified by flash column chromatography (dichloromethane) to give crude tert-butyl 4-(2-(3-(2-((5-methyl-4-(1-(2-methylbenzoyl)indolin-5-yl)thiazol-2-yl)amino)-2-oxoethyl)phenoxy)ethyl)piperazine-1-carboxylate (0.10 g, 14% yield) as a yellow solid. MS (ESI) m/z 696.4 [M+H]⁺.

### Step 2. N-(5-methyl-4-(1-(2-methylbenzoyl)indolin-5-yl)thiazol-2-yl)-2-(3-(2-(piperazin-1-yl)ethoxy)phenyl)acetamide

A solution of tert-butyl 4-(2-(3-(2-((5-methyl-4-(1-(2-methylbenzoyl)indolin-5-yl)thiazol-2-yl)amino)-2-oxoethyl)phenoxy)ethyl)piperazine-1-carboxylate (0.10 g, 0.14 mmol) in hydrochloric acid (4.0 M in dioxane, 8 mL) was stirred at room temperature for 8 hrs. The solvent was removed and the residue was dissolved in dichloromethane (50 mL), washed with saturated sodium carbonate aqueous solution, dried over anhydrous sodium sulfate, filtered and concentrated in vacuo. The residue was purified by flash column chromatography (dichloromethane/methanol=1/20) to give crude N-(5-methyl-4-(1-(2-methylbenzoyl)indolin-5-yl)thiazol-2-yl)-2-(3-(2-(piperazin-1-yl)ethoxy)phenyl)acetamide (50.0 mg, 58% yield) as a yellow solid. MS (ESI) m/z 596.7 [M+H]⁺.

### Step 3. 2-(3-(2-(4-(2-(2,6-dioxopiperidin-3-yl)-1,3-dioxoisoindolin-5-yl)piperazin-1-yl)ethoxy) phenyl)-N-(5-methyl-4-(1-(2-methylbenzoyl)indolin-5-yl)thiazol-2-yl)acetamide

A solution of N-(5-methyl-4-(1-(2-methylbenzoyl)indolin-5-yl)thiazol-2-yl)-2-(3-(2-(piperazin-1-yl) ethoxy)phenyl)acetamide (40.0 mg, 0.067 mmol), 2-(2,6-dioxopiperidin-3-yl)-5-fluoroisoindoline-1,3-dione (18.5 mg, 0.067 mmol) and N,N-diisopropylethylamine (18.0 mg, 0.14 mmol) in 1-methylpyrrolidin-2-one (2 mL) was stirred at 140° C. for 18 hrs. The mixture was cooled to room temperature, diluted with water (10 mL), extracted with ethyl acetate (20 mL×4), dried over anhydrous sodium sulfate, filtered and concentrated in vacuo. The residue was purified by Prep-HPLC (Column: Welch Xtimate 21.2×250 mm C18, 10 μm, Mobile Phase: A: water (10 mmol/L ammonium bicarbonate) B: acetonitrile; B %: 30%-70% in 15.0 min) to give 2-(3-(2-(4-(2-(2,6-dioxopiperidin-3-yl)-1,3-dioxoisoindolin-5-yl)piperazin-1-yl)ethoxy)phenyl)-N-(5-methyl-4-(1-(2-methylbenzoyl)indolin-5-yl)thiazol-2-yl)acetamide (6.5 mg, 11% yield) as a yellow solid. ¹H NMR (400 MHz, DMSO-d₆): δ12.35 & 12.27 (s, 1H)*, 11.09 (s, 1H), 8.23 & 5.54* (d, J=8.4 Hz, 1H), 7.66 (t, J=8.4 Hz, 1H), 7.52 (s, 2H), 7.37-7.22 (m, 7H), 6.96 (s, 1H), 6.91-6.86 (m, 2H), 5.07 (dd, J=12.4 & 5.6 Hz, 1H), 4.21 & 3.70 (m, 2H)*, 4.12 (s, 2H), 3.74-3.71 (m, 2H), 3.44-3.38 (m, 3.13 (t, J=8.2 Hz, 2H), 2.89-2.83 (m, 1H), 2.78-2.75 (m, 2H), 2.67-2.63 (m, 2.56-2.54 (m, 2H), 2.45 & 2.34 (s, 3H)*, 2.29 & 2.18 (s, 3H)*, 2.02-1.99 (m, 1H). MS (ESI) m/z: 853.2 [M+H]⁺. *Multiple signals arising from conformational isomers.

### Example 26

### Synthesis of N-(4-(1-(cyclopropanecarbonyl)indolin-5-yl)-5-methylthiazol-2-yl)-2-(3-(2-(2-(2-(2,6-dioxopiperidin-3-yl)-1,3-dioxoisoindolin-5-ylamino)ethoxy)ethoxy)-4-fluorophenyl)acetamide (Cpd. No. 27)

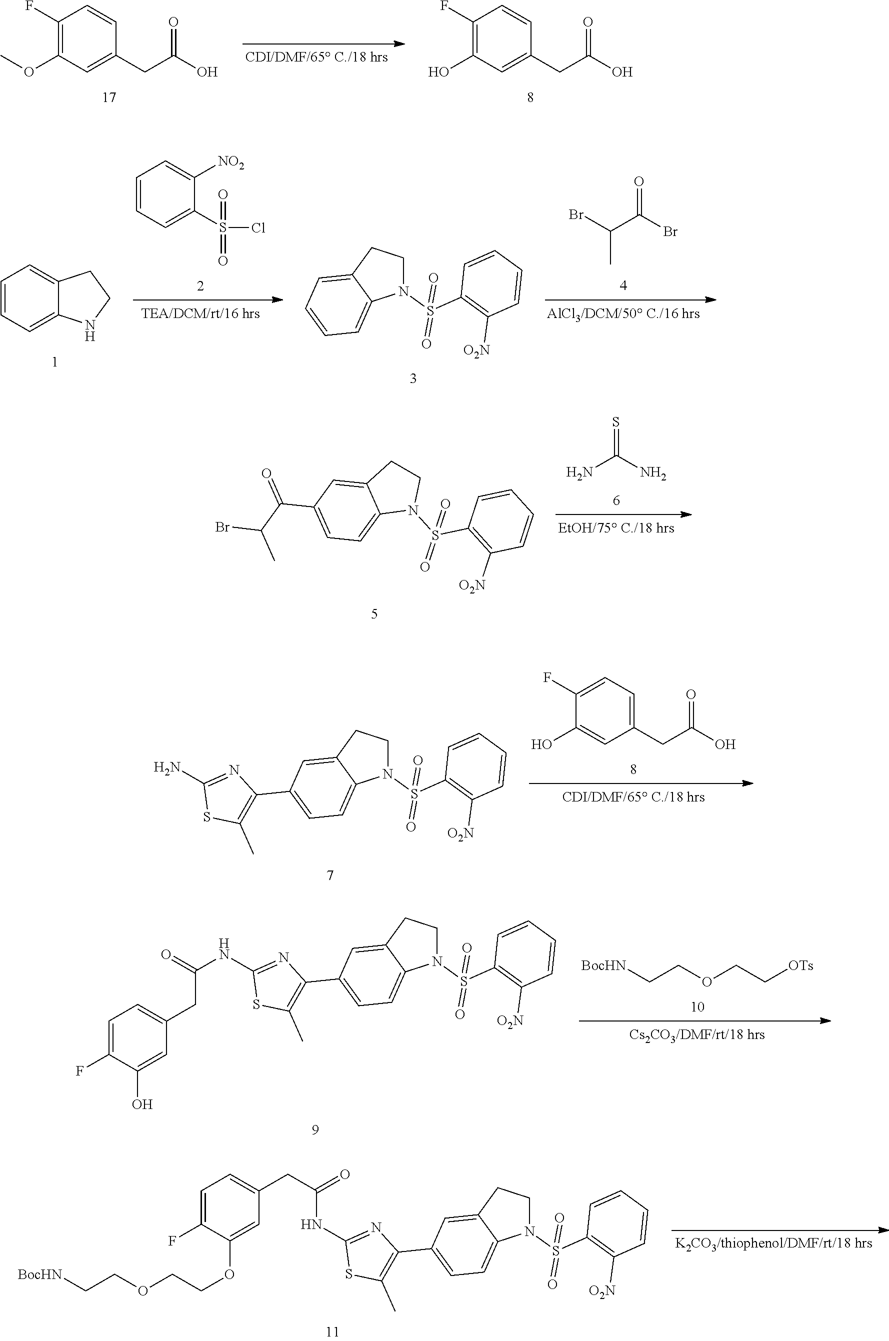

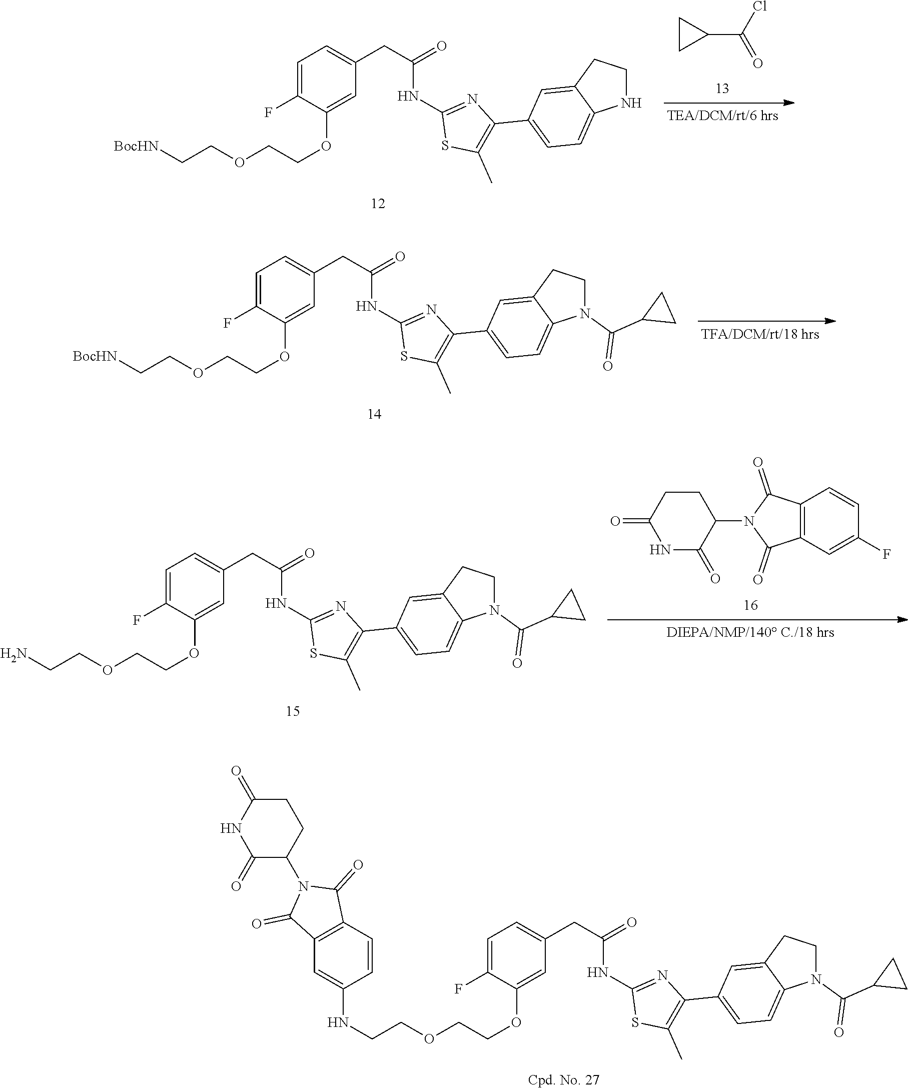 [SMILES: Cc1sc(NC(=O)Cc2ccc(F)c(OCCOCCNC(=O)OC(C)(C)C)c2)nc1-c1ccc2c(c1)CCN2 ; O=C(Cl)C1CC1 ; Cc1sc(NC(=O)Cc2ccc(F)c(OCCOCCNC(=O)OC(C)(C)C)c2)nc1-c1ccc2c(c1)CCN2C(=O)C1CC1 ; Cc1sc(NC(=O)Cc2ccc(F)c(OCCOCCN)c2)nc1-c1ccc2c(c1)CCN2C(=O)C1CC1 ; O=C1CCC(N2C(=O)c3ccc(F)cc3C2=O)C(=O)N1 ; Cc1sc(NC(=O)Cc2ccc(F)c(OCCOCCNc3ccc4c(c3)C(=O)N(C3CCC(=O)NC3=O)C4=O)c2)nc1-c1ccc2c(c1)CCN2C(=O)C1CC1]

### Step 1. 1-((2-nitrophenyl)sulfonyl)indoline

To a solution of indoline (25.00 g, 209.8 mmol) and triethylamine (63.68 g, 629.4 mmol) in dichloromethane (300 mL), was added 2-nitrobenzenesulfonyl chloride (46.49 g, 209.8 mmol) at 0° C. After addition, the mixture was stirred at room temperature for 16 hrs. The reaction mixture was diluted with water (100 mL), extracted with dichloromethane (250 mL), washed with brine (100 mL×2), dried over anhydrous sodium sulfate, filtered and concentrated in vacuo to give 1-((2-nitrophenyl)sulfonyl)indoline (54.0 g, 84.5% yield) as a yellow solid. MS (ESI) m/z: 305.0 [M+H]⁺.

### Step 2. 2-bromo-1-(1-((2-nitrophenyl)sulfonyl)indolin-5-yl)propan-1-one

1-((2-Nitrophenyl)sulfonyl)indoline (54.0 g, 177.4 mmol) and aluminium chloride (70.98 g, 532.3 mmol) was dissolved in dichloromethane (800 mL), then 2-bromopropanoyl bromide (114.95 g, 532.33 mmol) was added. The mixture was stirred at 50° C. for 16 hrs. The mixture was cooled to room temperature, quenched with water (500 mL), basified to give pH=8-10 with 6 N NaOH solution, extracted with dichloromethane (300 mL), dried over anhydrous sodium sulfate, filtered and concentrated in vacuo to give crude 2-bromo-1-(1-((2-nitrophenyl)sulfonyl)indolin-5-yl)propan-1-one (76.0 g, 97.5% yield) as a yellow oil. MS (ESI) m/z: 439.0 and 441.0 [M+H]⁺.

### Step 3. 5-methyl-4-(1-((2-nitrophenyl)sulfonyl)indolin-5-yl)thiazol-2-amine

A solution of 2-bromo-1-(1-((2-nitrophenyl)sulfonyl)indolin-5-yl)propan-1-one (76.0 g, 173 mmol) and thiourea (39.51 g, 519.0 mmol) in ethanol (800 mL) was stirred at 75° C. for 18 hrs. The mixture was cooled to room temperature, diluted with water (1.5 L), filtered. The filter cake was washed with water (100 mL×2), dried to give 5-methyl-4-(1-((2-nitrophenyl)sulfonyl)indolin-5-yl)thiazol-2-amine (45.12 g, 62.6% yield) as a yellow solid. MS (ESI) m/z: 417.1 [M+H]⁺.

### Step 4. 2-(4-fluoro-3-hydroxyphenyl)acetic acid

A solution of 2-(4-fluoro-3-methoxyphenyl)acetic acid (19.00 g, 103.2 mmol) in hydrobromic acid (40% hydrobromic acid in water, 50 mL) and acetic acid (50 mL) was stirred at 90° C. for 18 hrs. The solvent was removed in vacuo and the residue was dissolved in dichloromethane (200 mL), washed with brine (50 mL×2), dried over anhydrous sodium sulfate, filtered and concentrated to give 2-(4-fluoro-3-hydroxyphenyl)acetic acid (15.0 g, 85.5%) as a yellow solid. MS (ESI) m/z: 171.0 [M+H]⁺.

### Step 5. 2-(4-fluoro-3-hydroxyphenyl)-N-(5-methyl-4-(1-((2-nitrophenyl)sulfonyl)indolin-5-yl)thiazol-2-yl)acetamide

A solution of 2-(4-fluoro-3-hydroxyphenyl)acetic acid (2.86 g, 16.8 mmol), 1,1′-carbonyldiimidazole (4.09 g, 25.2 mmol) and N,N-diisopropylethylamine (6.51 g, 50.4 mmol) in N,N-dimethylformamide (40 mL) was stirred at room temperature for 30 min. 5-Methyl-4-(1-((2-nitrophenyl)sulfonyl)indolin-5-yl)thiazol-2-amine (7.00 g, 16.8 mmol) was added into the resulting mixture. The mixture was stirred at 65° C. for 18 hrs. The mixture was cooled to room temperature, diluted with water (100 mL), extracted with dichloromethane (50 mL×2), dried over anhydrous sodium sulfate, filtered and concentrated in vacuo. The residue was purified by flash column chromatography (dichloromethane/methanol=1/20) to give 2-(4-fluoro-3-hydroxyphenyl)-N-(5-methyl-4-(1-(2-nitrophenylsulfonyl)indolin-5-yl)thiazol-2-yl)acetamide (5.20 g, 54.4% yield) as a yellow solid. MS (ESI) m/z 569.0 [M+H]⁺.

### Step 6. tert-butyl 2-(2-(2-fluoro-5-(2-(5-methyl-4-(1-(2-nitrophenylsulfonyl)indolin-5-yl)thiazol-2-ylamino)-2-oxoethyl)phenoxy)ethoxy)ethylcarbamate

A solution of 2-(4-fluoro-3-hydroxyphenyl)-N-(5-methyl-4-(1-((2-nitrophenyl)sulfonyl)indolin-5-yl)thiazol-2-yl)acetamide (5.00 g, 8.79 mmol), 2-(2-((tert-butoxycarbonyl)amino)ethoxy)ethyl 4-methylbenzenesulfonate (3.16 g, 8.79 mmol), cesium carbonate (5.73 g, 17.59 mmol) in N,N-dimethylformamide (30 mL) was stirred at room temperature for 18 hrs. The mixture was diluted with water (50 ml), extracted with dichloromethane (50 mL×2), dried over anhydrous sodium sulfate, filtered and concentrated in vacuo. The residue was purified by flash column chromatography (dichloromethane/methanol=1/20) to give tert-butyl (2-(2-(2-fluoro-5-(2-((5-methyl-4-(1-((2-nitrophenyl)sulfonyl)indolin-5-yl)thiazol-2-yl)amino)-2-oxoethyl)phenoxy)ethoxy)ethyl)carbamate (3.61 g, 54.3% yield) as a yellow solid. MS (ESI) m/z 756.2 [M+H]⁺.

### Step 7. tert-butyl 2-(2-(2-fluoro-5-(2-(4-(indolin-5-yl)-5-methylthiazol-2-ylamino)-2-oxoethyl)phenoxy)ethoxy)ethylcarbamate

A solution of tert-butyl (2-(2-(2-fluoro-5-(2-((5-methyl-4-(1-((2-nitrophenyl)sulfonyl)indolin-5-yl)thiazol-2-yl)amino)-2-oxoethyl)phenoxy)ethoxy)ethyl)carbamate (3.50 g, 4.63 mmol), potassium carbonate (1.28 g, 9.26 mmol), thiophenol (0.72 g, 5.56 mmol) in N,N-dimethylformamide (20 mL) was stirred at room temperature for 18 hrs. The reaction mixture was diluted with water (40 mL), extracted with dichloromethane (50 mL×2), dried over anhydrous sodium sulfate, filtered and concentrated in vacuo. The residue was purified by flash column chromatography (dichloromethane/methanol=20/1) to give tert-butyl 2-(2-(2-fluoro-5-(2-(4-(indolin-5-yl)-5-methylthiazol-2-ylamino)-2-oxoethyl)phenoxy)ethoxy)ethylcarbamate (2.42 g, 91.5% yield) as a yellow solid. MS (ESI) m/z: 571.3 [M+H]⁺.

### Step 8. tert-butyl 2-(2-(5-(2-(4-(1-(cyclopropanecarbonyl)indolin-5-yl)-5-methylthiazol-2-ylamino)-2-oxoethyl)-2-fluorophenoxy)ethoxy)ethylcarbamate

To a solution of tert-butyl 2-(2-(2-fluoro-5-(2-(4-(indolin-5-yl)-5-methylthiazol-2-ylamino)-2-oxoethyl)phenoxy)ethoxy)ethylcarbamate (0.60 g, 1.05 mmol) and triethylamine (0.32 g, 3.15 mmol) in dichloromethane (15 mL), was added cyclopropanecarbonyl chloride (0.12 g, 1.10 mmol) at 0° C. The mixture was stirred at room temperature for 6 hrs. The mixture was quenched with water (10 mL), extracted with dichloromethane (30 mL), dried over anhydrous sodium sulfate, filtered and concentrated in vacuo. The residue was purified by flash column chromatography (dichloromethane/methanol=20/1) to give tert-butyl 2-(2-(5-(2-(4-(1-(cyclopropanecarbonyl)indolin-5-yl)-5-methylthiazol-2-ylamino)-2-oxoethyl)-2-fluorophenoxy)ethoxy)ethylcarbamate (0.55 g, 82% yield) as a yellow solid. MS (ESI) m/z 639.3 [M+H]⁺.

### Step 9. 2-(3-(2-(2-aminoethoxy)ethoxy)-4-fluorophenyl)-N-(4-(1-(cyclopropanecarbonyl)indolin-5-yl)-5-methylthiazol-2-yl)acetamide

A solution of tert-butyl 2-(2-(5-(2-(4-(1-(cyclopropanecarbonyl)indolin-5-yl)-5-methylthiazol-2-ylamino)-2-oxoethyl)-2-fluorophenoxy)ethoxy)ethylcarbamate (0.50 g, 0.78 mmol) and trifluoroacetic acid (3 mL) in dichloromethane (5 mL) was stirred at room temperature for 18 hrs. The solvent was removed in vacuo and the residue was dissolved in dichloromethane (40 mL), washed with saturated sodium carbonate aqueous solution, dried over anhydrous sodium, filtered and concentrated in vacuo. The residue was purified by flash column chromatography (dichloromethane/methanol=1/10) to give 2-(3-(2-(2-aminoethoxy)ethoxy)-4-fluorophenyl)-N-(4-(1-(cyclopropanecarbonyl)indolin-5-yl)-5-methylthiazol-2-yl)acetamide (0.35 g, 83% yield) as a yellow solid. MS (ESI) m/z 539.1 [M+H]⁺.

### Step 10. N-(4-(1-(cyclopropanecarbonyl)indolin-5-yl)-5-methylthiazol-2-yl)-2-(3-(2-(2-(2-(2,6-dioxopiperidin-3-yl)-1,3-dioxoisoindolin-5-ylamino)ethoxy)ethoxy)-4-fluorophenyl)acetamide

A solution of 2-(3-(2-(2-aminoethoxy)ethoxy)-4-fluorophenyl)-N-(4-(1-(cyclopropanecarbonyl)indolin-5-yl)-5-methylthiazol-2-yl)acetamide (100 mg, 0.19 mmol), 2-(2,6-dioxopiperidin-3-yl)-5-fluoroisoindoline-1,3-dione (51.1 mg, 0.19 mmol) and N,N-diisopropylethylamine (72.0 mg, 0.56 mmol) in 1-methylpyrrolidin-2-one (2 mL) was stirred at 140° C. for 18 hrs. The mixture was diluted with water (10 mL), extracted with ethyl acetate (20 mL×4), dried over anhydrous sodium sulfate, filtered and concentrated. The residue was purified by Prep-HPLC (Column: Welch Xtimate 21.2*250 mm C18, 10 μm, Mobile Phase: A: water (10 mmol/L ammonium bicarbonate) B: acetonitrile; B %: 30%-70% in 15.0 min) to give N-(4-(1-(cyclopropanecarbonyl)indolin-5-yl)-5-methylthiazol-2-yl)-2-(3-(2-(2-(2-(2,6-dioxopiperidin-3-yl)-1,3-dioxoisoindolin-5-ylamino)ethoxy)ethoxy)-4-fluorophenyl)acetamide (18.1 mg, 12% yield) as a yellow solid. ¹H NMR (400 MHz, DMSO-d₆): δ 12.29 (s, 1H), 11.06 (s, 1H), 8.07 (d, J=7.2 Hz, 1H), 7.54 (d, J=8.4 Hz, 2H), 7.49 (s, 1H), 7.42 (d, J=8.4 Hz, 1H), 7.18-7.13 (m, 3H), 6.90-6.87 (m, 2H), 5.03 (dd, J=13.0 & 5.2, 1H), 4.33 (m, 2H), 4.19 (t, J=4.4 Hz, 2H), 3.81 (t, J=4.4 Hz, 2H), 3.71 (s, 2H), 3.67 (t, J=5.4 Hz, 2H), 3.40-3.35 (m, 2H), 3.24 (m, 2H), 2.91-2.81 (m, 1H), 2.59-2.51 (m, 2H), 2.43 (s, 3H), 1.99-1.96 (m, 2H), 0.88-0.86 (m, 4H). MS (ESI) m/z: 759.2 [M+H]⁺.

### Example 27

### Synthesis of 2-(3-(3-(4-(2-(2,6-dioxopiperidin-3-yl)-1,3-dioxoisoindolin-5-yl)piperazin-1-yl)propoxy)phenyl)-N-(5-methyl-4-(1-(2-methylbenzoyl)indolin-5-yl)thiazol-2-yl)acetamide (Cpd No. 25)

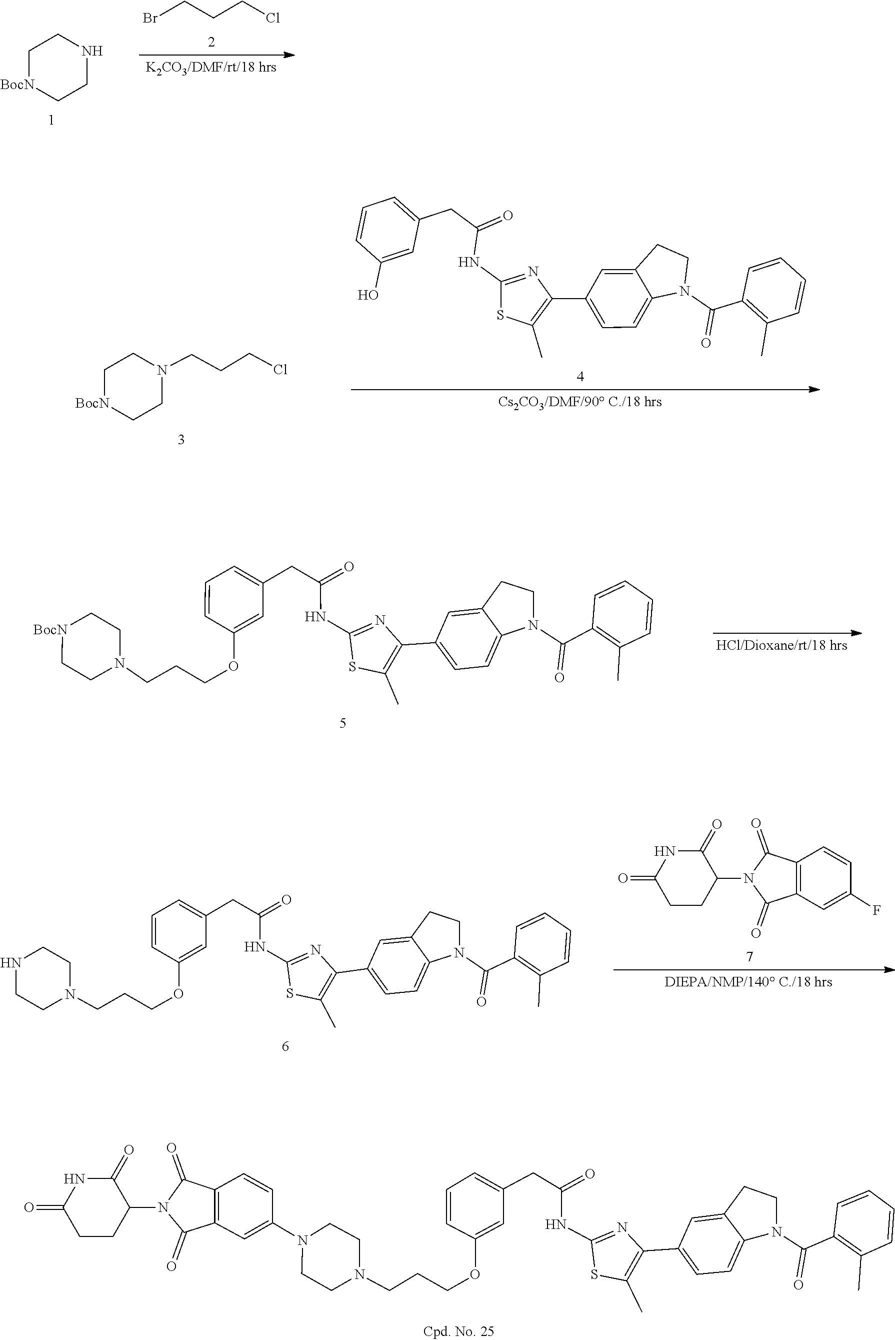

### Step 1. tert-butyl 4-(3-chloropropyl)piperazine-1-carboxylate

A solution of tert-butyl piperazine-1-carboxylate (3.00 g, 16.1 mmol), 1-bromo-3-chloropropane (2.54 g, 16.1 mmol), potassium carbonate (4.45 g, 32.2 mmol) in N,N-dimethylformamide (40 mL) was stirred at room temperature for 18 hrs. The mixture was diluted with water (100 mL), extracted with ethyl acetate (200 mL×2), dried over anhydrous sodium sulfate, filtered and concentrated in vacuo. The residue was purified by flash column chromatography (petroleum ether/ethyl acetate=1/1) to give tert-butyl 4-(3-chloropropyl)piperazine-1-carboxylate (2.0 g, 47% yield) as a colorless oil. MS (ESI) m/z 263.1 [M+H]⁺

### Step 2. tert-butyl 4-(3-(3-(2-((5-methyl-4-(1-(2-methylbenzoyl)indolin-5-yl)thiazol-2-yl)amino)-2-oxoethyl)phenoxy)propyl)piperazine-1-carboxylate

A solution of tert-butyl 4-(3-chloropropyl)piperazine-1-carboxylate (0.50 g, 1.9 mmol), 2-(3-hydroxyphenyl)-N-(5-methyl-4-(1-(2-methylbenzoyl)indolin-5-yl)thiazol-2-yl)acetamide (0.92 g, 1.9 mmol) and cesium carbonate (1.86 g, 5.70 mmol) in N,N-dimethylformamide (10 mL) was stirred at 90° C. for 18 hrs. The mixture was diluted with water (25 mL), extracted with dichloromethane (50 mL×2), dried over anhydrous sodium sulfate, filtered and concentrated in vacuo. The residue was purified by flash column chromatography (dichloromethane/methanol=1/10) to give tert-butyl 4-(3-(3-(2-((5-methyl-4-(1-(2-methylbenzoyl)indolin-5-yl)thiazol-2-yl)amino)-2-oxoethyl)phenoxy)propyl)piperazine-1-carboxylate (0.14 g, 10.4% yield) as a yellow solid. MS (ESI) m/z 710.3 [M+H]⁺

### Step 3. N-(5-methyl-4-(1-(2-methylbenzoyl)indolin-5-yl)thiazol-2-yl)-2-(3-(3-(piperazin-1-yl)propoxy)phenyl)acetamide

A solution of tert-butyl 4-(3-(3-(2-((5-methyl-4-(1-(2-methylbenzoyl)indolin-5-yl)thiazol-2-yl) amino)-2-oxoethyl)phenoxy)propyl)piperazine-1-carboxylate (0.14 g, 0.20 mmol) in hydrochloric acid (4 M in 1,4-dioxane, 4 mL) and 1,4-dioxane (2 mL) was stirred at room temperature for 18 hrs. The solvent was removed and the residue was dissolved in dichloromethane (100 mL), washed with saturated sodium carbonate aqueous solution, dried over anhydrous sodium, filtered and concentrated in vacuo. The residue was purified by flash column chromatography (dichloromethane/methanol=1/10) to give N-(5-methyl-4-(1-(2-methylbenzoyl)indolin-5-yl)thiazol-2-yl)-2-(3-(3-(piperazin-1-yl)propoxy) phenyl)acetamide (0.11 g, 91% yield) as a yellow solid. MS (ESI) m/z 610.3 [M+H]⁺

### Step 4. 2-(3-(3-(4-(2-(2,6-dioxopiperidin-3-yl)-1,3-dioxoisoindolin-5-yl)piperazin-1-yl)propoxy)phenyl)-N-(5-methyl-4-(1-(2-methylbenzoyl)indolin-5-yl)thiazol-2-yl)acetamide

A solution of N-(5-methyl-4-(1-(2-methylbenzoyl)indolin-5-yl)thiazol-2-yl)-2-(3-(3-(piperazin-1-yl) propoxy)phenyl)acetamide (0.11 g, 0.18 mmol), 2-(2,6-dioxopiperidin-3-yl)-5-fluoroisoindoline-1,3-dione (54.8 mg, 0.20 mmol) and N,N-diisopropylethylamine (69.9 mg, 0.54 mmol) in N-methylpyrrolidone (5 mL) was stirred at 140° C. for 18 hrs. The mixture was cooled to room temperature, diluted with water (10 mL), extracted with ethyl acetate (20 mL×2), dried over anhydrous sodium sulfate, filtered and concentrated in vacuo. The residue was purified by Prep-HPLC (Column: Welch Xtimate 21.2*250 mm C18, 10 μm, Mobile Phase: A: water (10 mmol/L ammonium bicarbonate) B: acetonitrile; B %: 30%-70% in 15.0 min) to give 2-(3-(3-(4-(2-(2,6-dioxopiperidin-3-yl)-1,3-dioxoisoindolin-5-yl)piperazin-1-yl)propoxy)phenyl)-N-(5-methyl-4-(1-(2-methylbenzoyl)indolin-5-yl)thiazol-2-yl)acetamide (16.7 mg, 10.7% yield) as a yellow solid. ¹H NMR (400 MHz, DMSO-d₆): δ 12.38 (s, 1H), 11.09 (s, 1H), 8.23 & 5.55* (d, J=8.4 Hz, 1H), 7.65 (d, J=8.8 Hz, 1H), 7.51 (s, 2H), 7.37-7.21 (m, 7H), 6.93-6.83 (m, 3H), 5.07 (dd, J=12.8 & 5.2 Hz, 1H), 4.2 & 3.7* (m, 2H), 4.02 (m, 2H), 3.75-3.70 (m, 2H), 3.43 (s, 4H), 3.14-3.10 (m, 2H), 2.89-2.84 (m, 1H), 2.60-2.56 (m, 2H), 2.55-2.52 (m, 2.49 (s, 2H), 2.44 & 2.3* (s, 3H), 2.29 & 2.18* (s, 3H), 2.02-1.98 (m, 1H), 1.93-1.89 (m, 2H). MS (ESI) m/z: 866.2 [M+H]⁺. *Multiple signals arising from conformational isomers.

### Example 28

### Synthesis of 2-(3-(4-(4-(2-(2,6-dioxopiperidin-3-yl)-1,3-dioxoisoindolin-5-yl)piperazin-1-yl)butoxy)phenyl)-N-(5-methyl-4-(1-(2-methylbenzoyl)indolin-5-yl)thiazol-2-yl)acetamide (Cpd. No. 26)

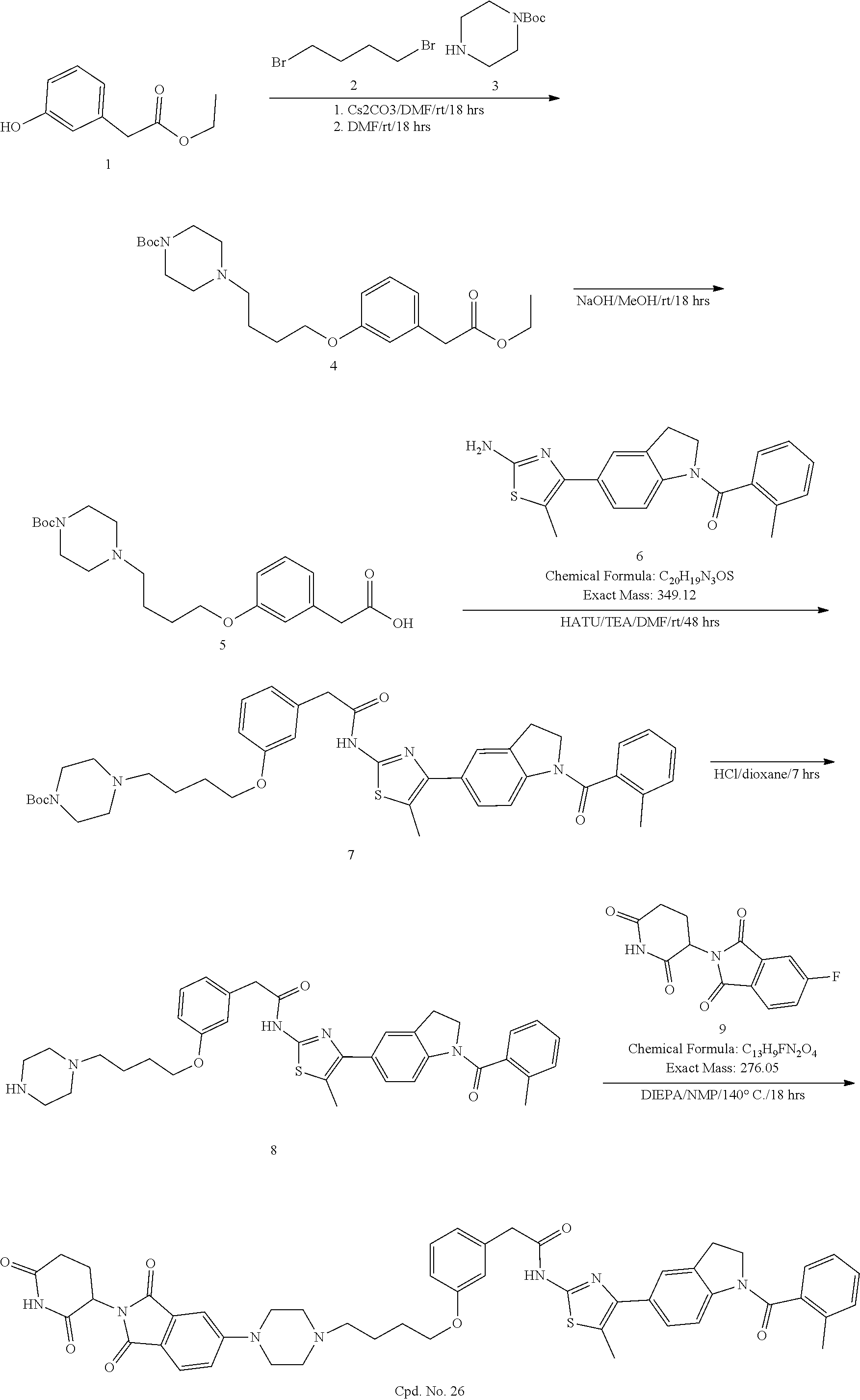

### Step 1. tert-butyl 4-(4-(3-(2-ethoxy-2-oxoethyl)phenoxy)butyl)piperazine-1-carboxylate

A solution of ethyl 2-(3-hydroxyphenyl)acetate (1.50 g, 8.32 mmol), 1,4-dibromobutane (1.80 g, 8.32 mmol), cesium carbonate (8.14 g, 24.97 mmol) in N,N-dimethylformamide (20 mL) was stirred at room temperature for 18 hrs. Tert-butyl piperazine-1-carboxylate (1.55 g, 8.31 mmol) was added into the resulting mixture. The mixture was stirred at room temperature for 18 hrs. The mixture was quenched with water (50 mL), extracted with dichloromethane (100 mL×2), washed with brine (30 mL×2), dried over anhydrous sodium sulfate, filtered and concentrated in vacuo. The residue was purified by Prep-HPLC (Column: Welch Xtimate 21.2*250 mm C18, 10 μm, Mobile Phase: A: water (10 mmol/L ammonium bicarbonate) B: acetonitrile; B %: 30%-70% in 15.0 min) to give tert-butyl 4-(4-(3-(2-ethoxy-2-oxoethyl)phenoxy)butyl)piperazine-1-carboxylate (0.40 g, 11.5% yield) as a white solid. MS (ESI) m/z 421.3 [M+H]⁺

### Step 2. 2-(3-(4-(4-(tert-butoxycarbonyl)piperazin-1-yl)butoxy)phenyl)acetic acid

A solution of tert-butyl 4-(4-(3-(2-ethoxy-2-oxoethyl)phenoxy)butyl)piperazine-1-carboxylate (0.40 g, 0.95 mmol), sodium hrdroxide (0.11 g, 2.85 mmol) in water (5 mL) and methanol (20 mL) was stirred at room temperature for 18 hrs. The mixture was acidified to give pH=1-3 with 2 M hydrochloric acid aqueous solution, extracted with dichloromethane (50 mL×3), dried over anhydrous sodium sulfate, filtered and concentrated in vacuo. The residue was purified by Prep-HPLC (Column: Welch Xtimate 21.2*250 mm C18, 10 pin, Mobile Phase: A: water (10 mmol/L ammonium bicarbonate) B: acetonitrile; B %: 30%-70% in 15.0 min) to give 2-(3-(4-(4-(tert-butoxycarbonyl)piperazin-1-yl)butoxy)phenyl)acetic acid (0.26 g, 69.8% yield) as a white solid. MS (ESI) m/z 393.4 [M+H]⁺

### Step 3. tert-butyl 4-(4-(3-(2-((5-methyl-4-(1-(2-methylbenzoyl)indolin-5-yl)thiazol-2-yl)amino)-2-oxoethyl)phenoxy)butyl)piperazine-1-carboxylate

A solution of 2-(3-(4-(4-(tert-butoxycarbonyl)piperazin-1-yl)butoxy)phenyl)acetic acid (0.26 g, 0.66 mmol), (5-(2-amino-5-methylthiazol-4-yl)indolin-1-yl)(o-tolyl)methanone (0.46 g, 1.32 mmol), 2-(7-azabenzotriazol-1-yl)-N,N,N′,N′-tetramethyluronium hexafluorophosphate (0.50 g, 1.32 mmol) and triethylamine (0.27 g, 2.65 mmol) in N,N-dimethylformamide (10 mL) was stirred at room temperature for 48 hrs. The mixture was diluted with water (50 mL), extracted with dichloromethane (50 mL×2), dried over anhydrous sodium sulfate, filtered and concentrated in vacuo. The residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15.0 min) to give tert-butyl 4-(4-(3-(2-((5-methyl-4-(1-(2-methylbenzoyl)indolin-5-yl)thiazol-2-yl)amino)-2-oxoethyl)phenoxy)butyl)piperazine-1-carboxylate (0.11 g, 23.0% yield) as a white solid. MS (ESI) m/z 724.4 [M+H]⁺

### Step 4. N-(5-methyl-4-(1-(2-methylbenzoyl)indolin-5-yl)thiazol-2-yl)-2-(3-(4-(piperazin-1-yl)butoxy)phenyl)acetamide

A solution of tert-butyl 4-(4-(3-(2-((5-methyl-4-(1-(2-methylbenzoyl)indolin-5-yl)thiazol-2-yl)amino)-2-oxoethyl)phenoxy)butyl)piperazine-1-carboxylate (0.11 g, 0.15 mmol) and hydrochloric acid (4 M in dioxane, 8 mL) in dioxane (8 mL) was stirred at room temperature for 7 hrs. The solvent was removed in vacuo and the residue was dissolved in dichloromethane (80 mL), washed with saturated sodium carbonate aqueous solution, dried over anhydrous sodium sulfate, filtered and concentrated in vacuo to give N-(5-methyl-4-(1-(2-methylbenzoyl)indolin-5-yl)thiazol-2-yl)-2-(3-(4-(piperazin-1-yl)butoxy)phenyl)acetamide (70.0 mg, 74.8%) as a yellow solid. MS (ESI) m/z 624.3 [M+H]⁺

### Step 5. 2-(3-(4-(4-(2-(2,6-dioxopiperidin-3-yl)-1,3-dioxoisoindolin-5-yl)piperazin-1-yl)butoxy)phenyl)-N-(5-methyl-4-(1-(2-methylbenzoyl)indolin-5-yl)thiazol-2-yl)acetamide

A solution of N-(5-methyl-4-(1-(2-methylbenzoyl)indolin-5-yl)thiazol-2-yl)-2-(3-(4-(piperazin-1-yl)butoxy)phenyl)acetamide (0.070 g, 0.11 mmol), 2-(2,6-dioxopiperidin-3-yl)-5-fluoroisoindoline-1,3-dione (46.1 mg, 0.17 mmol), N,N-diisopropylethylamine (43.9 mg, 0.34 mmol) in 1-methylpyrrolidin-2-one (2 mL) was stirred at 140° C. for 18 hrs. The mixture was cooled to room temperature, diluted with water (10 mL), extracted with dichloromethane (10 mL×2), dried over anhydrous sodium sulfate, filtered and concentrated in vacuo. The residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15.0 min) to give 2-(3-(4-(4-(2-(2,6-dioxopiperidin-3-yl)-1,3-dioxoisoindolin-5-yl)piperazin-1-yl)butoxy)phenyl)-N-(5-methyl-4-(1-(2-methylbenzoyl)indolin-5-yl)thiazol-2-yl)acetamide (55.0 mg, 55.7% yield) as a yellow solid. ¹H NMR (400 MHz, DMSO-d₆): δ 12.34 & 12.25* (s, 1H), 11.09 (s, 1H), 8.23 & 5.52* (d, J=8.8 Hz, 1H), 7.66 (d, J=8.0 Hz, 1H), 7.52 (s, 1H), 7.52 & 7.04* (m, 1H), 7.37-7.30 (m, 5H), 7.24-7.21 (m, 2H), 6.92-6.83 (m, 3H), 5.07 (dd, J=12.6 & 5.4 Hz, 1H), 4.21 & 3.73* (m, 2H), 4.0 (t, J=5.8 Hz, 2H), 3.70 (s, 2H), 3.41-3.37 (m, 4H), 3.12 (t, J=8.4 Hz, 2H), 2.91-2.83 (m, 1H), 2.60-2.51 (m, 2H), 2.48 (m, 2.45 & 2.34* (s, 3H), 2.38 (t, J=6.8 Hz, 2H), 2.29 & 2.18* (s, 3H), 2.02-2.00 (m, 1H), 1.76-1.75 (m, 2H), 1.65-1.60 (m, 2H). MS (ESI) m/z: 880.3 [M+H]⁺. *Multiple signals arising from conformational isomers.

### Example 29

### Synthesis of N-(4-(1-(cyclopropanecarbonyl)indolin-5-yl)-5-methylthiazol-2-yl)-2-(3-(2-(2-(2-(2,6-dioxopiperidin-3-yl)-1-oxoisoindolin-5-ylamino)ethoxy)ethoxy)-4-fluorophenyl)acetamide (Cpd. No. 28)

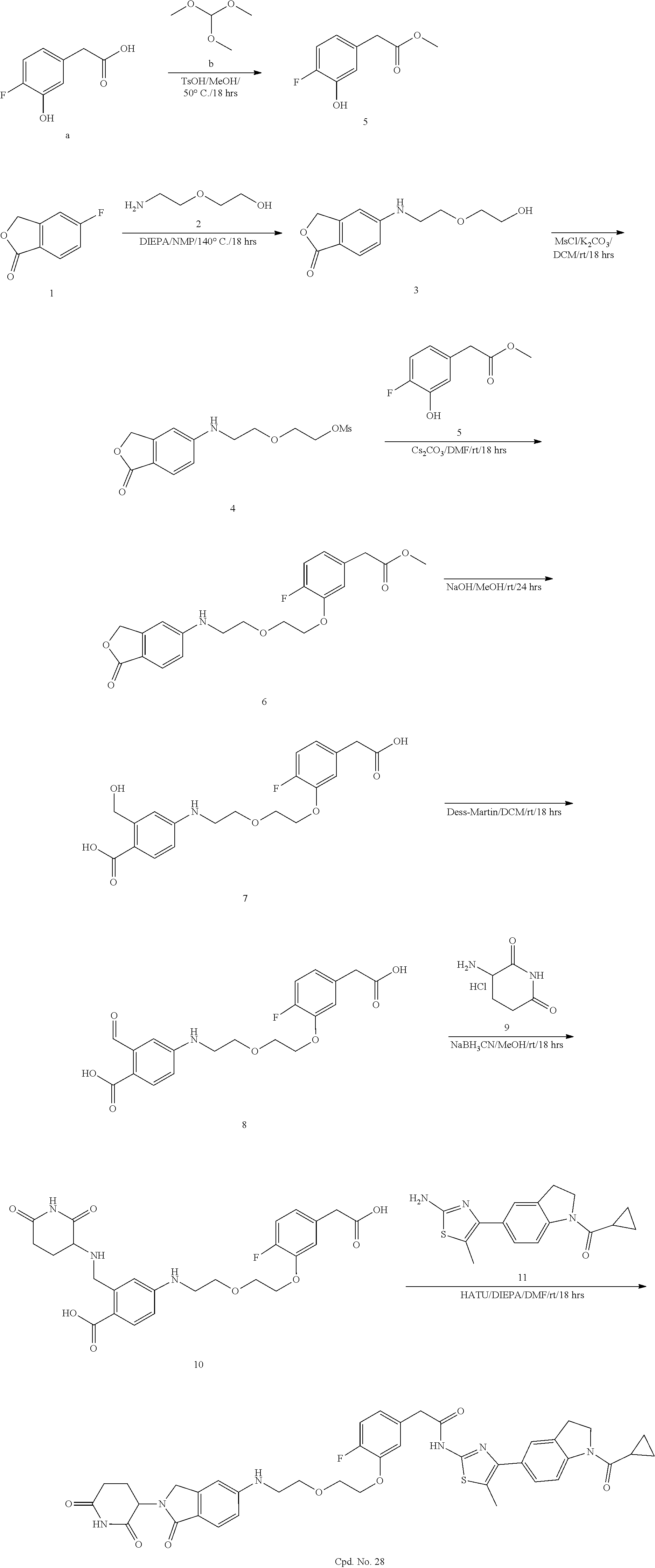

### Step 1. 5-(2-(2-hydroxyethoxy)ethylamino)isobenzofuran-1(3H)-one

A solution of 5-fluoroisobenzofuran-1(3H)-one (5.00 g, 32.9 mmol), 2-(2-aminoethoxy)ethanol (4.15 g, 39.4 mmol) and N,N-diisopropylethylamine (12.74 g, 98.61 mmol) in 1-methylpyrrolidin-2-one (50 mL) was stirred at 140° C. for 18 hrs. The mixture was cooled to room temperature, diluted with water (50 mL), extracted with dichloromethane (25 mL×8), dried over anhydrous sodium sulfate, filtered and concentrated in vacuo. The residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15 min) to give 5-(2-(2-hydroxyethoxy)ethylamino)isobenzofuran-1(3H)-one (5.10 g, 65.3% yield) as a white solid. MS (EST) m/z: 237.9 [M+H]⁺.

### Step 2. 2-(2-(1-oxo-1,3-dihydroisobenzofuran-5-ylamino)ethoxy)ethyl methanesulfonate

To a solution of 5-(2-(2-hydroxyethoxy)ethylamino)isobenzofuran-1(3H)-one (5.00 g, 21.1 mmol) and potassium carbonate (11.65 g, 84.30 mmol) in dichloromethane (50 mL) was added methanesulfonyl chloride (2.90 g, 25.29 mmol) at 0° C. After addition, the mixture was stirred at room temperature for 18 hrs. Then another of methanesulfonyl chloride (3.62 g, 31.61 mmol) was added at room temperature, the mixture was stirred at room temperature for another 18 hrs. The mixture was diluted with water (50 mL), extracted with dichloromethane (50 mL×3), washed with brine (40 mL), dried over anhydrous sodium sulfate, filtered and concentrated in vacuo to give 2-(2-(1-oxo-1,3-dihydroisobenzofuran-5-ylamino)ethoxy)ethyl methanesulfonate (3.80 g, 57.1% yield) as an off-white solid. MS (ESI) m/z: 315.9 [M+H]⁺.

### Step 3a. methyl 2-(4-fluoro-3-hydroxyphenyl)acetate

A solution of 2-(4-fluoro-3-hydroxyphenyl)acetic acid (3.00 g, 17.6 mmol), trimethoxymethane (3.74 g, 35.3 mmol) and 4-methylbenzenesulfonic acid (0.30 g, 1.8 mmol) in methanol (30 mL) was stirred at 50° C. for 18 hrs. The mixture was diluted with water (30 mL), extracted with dichloromethane (20 mL×3), dried over anhydrous sodium sulfate, filtered and concentrated in vacuo. The residue was purified by flash column chromatography (ethyl acetate/petroleum ether=5/1) to give methyl 2-(4-fluoro-3-hydroxyphenyl)acetate (1.90 g, 58.5% yield) as a white solid. MS (ESI) m/z: 185.1 [M+H]⁺.

### Step 3. methyl 2-(4-fluoro-3-(2-(2-(1-oxo-1,3-dihydroisobenzofuran-5-ylamino)ethoxy)ethoxy)phenyl)acetate

A solution of 2-(2-(1-oxo-1,3-dihydroisobenzofuran-5-ylamino)ethoxy)ethyl methanesulfonate (3.20 g, 10.1 mmol), methyl 2-(4-fluoro-3-hydroxyphenyl)acetate (1.87 g, 10.1 mmol) and cesium carbonate (13.23 g, 40.59 mmol) in N,N-dimethylformamide (20 mL) was stirred at room temperature for 18 hrs. The mixture was diluted with water (40 mL), extracted with dichloromethane (30 mL×3), washed with brine (30 mL), dried over anhydrous sodium sulfate, filtered and concentrated in vacuo to give crude methyl 2-(4-fluoro-3-(2-(2-(1-oxo-1,3-dihydroisobenzofuran-5-ylamino)ethoxy)ethoxy)phenyl)acetate (4.20 g, overweight) as a yellow solid. MS (ESI) m/z: 404.0 [M+H]⁺.

### Step 4. 4-(2-(2-(5-(carboxymethyl)-2-fluorophenoxy)ethoxy)ethylamino)-2-(hydroxymethyl)benzoic acid

A solution of methyl 2-(4-fluoro-3-(2-(2-(1-oxo-1,3-dihydroisobenzofuran-5-ylamino)ethoxy)ethoxy)phenyl) acetate (4.20 g, 10.1 mmol maximum) and sodium hydroxide (3.33 g, 83.29 mmol) in methanol (20 mL) and water (20 mL) was stirred at room temperature for 24 hrs. The mixture was diluted with water (20 mL), acidified with aqueous hydrochloric acid (2 M) solution to pH=3, extracted with dichloromethane (30 mL×5), dried over anhydrous sodium sulfate, filtered and concentrated in vacuo. The residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15 min) to give 4-(2-(2-(5-(carboxymethyl)-2-fluorophenoxy)ethoxy)ethylamino)-2-(hydroxymethyl)benzoic acid (3.10 g, 75.3% yield) as a white solid. MS (ESI) m/z: 408.1 [M+H]⁺.

### Step 5. 4-(2-(2-(5-(carboxymethyl)-2-fluorophenoxy)ethoxy)ethylamino)-2-formylbenzoic acid

A solution of 4-(2-(2-(5-(carboxymethyl)-2-fluorophenoxy)ethoxy)ethylamino)-2-(hydroxymethyl)benzoic acid (1.50 g, 3.68 mmol) and Dess-martin reagent (2.34 g, 5.52 mmol) in dichloromethane (20 mL) was stirred at room temperature for 18 hrs. The mixture was diluted with water (20 mL), extracted with dichloromethane (30 mL×3), dried over anhydrous sodium sulfate, filtered and concentrated in vacuo. The residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15 min) to give 4-(2-(2-(5-(carboxymethyl)-2-fluorophenoxy)ethoxy)ethylamino)-2-formylbenzoic acid (0.90 g, 60% yield) as a white solid. MS (ESI) m/z: 406.2 [M+H]⁺.

### Step 6. 4-(2-(2-(5-(carboxymethyl)-2-fluorophenoxy)ethoxy)ethylamino)-2-((2,6-dioxopiperidin-3-ylamino)methyl)benzoic acid

To a solution of 4-(2-(2-(5-(carboxymethyl)-2-fluorophenoxy)ethoxy)ethylamino)-2-formylbenzoic acid (0.89 g, 2.2 mmol) and 3-aminopiperidine-2,6-dione hydrochloride (0.54 g, 3.3 mmol) in methanol (30 mL) was added sodium acetate (0.72 g, 8.78 mmol) and sodium borohydride (0.41 g, 6.59 mmol) at room temperature. After addition, the mixture was stirred at room temperature for 18 hrs. The mixture was diluted with water (20 mL), extracted with dichloromethane (30 mL×3), dried over anhydrous sodium sulfate, filtered and concentrated in vacuo. The residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15 min) to give 4-(2-(2-(5-(carboxymethyl)-2-fluorophenoxy)ethoxy)ethylamino)-2-((2,6-dioxopiperidin-3-ylamino)methyl)benzoic acid (0.42 g, 37% yield) as a white solid. MS (ESI) m/z: 517.9 [MH]⁺.

### Step 7. N-(4-(1-(cyclopropanecarbonyl)indolin-5-yl)-5-methylthiazol-2-yl)-2-(3-(2-(2-(2-(2,6-dioxopiperidin-3-yl)-1-oxoisoindolin-5-ylamino)ethoxy)ethoxy)-4-fluorophenyl)acetamide

A solution of 4-(2-(2-(5-(carboxymethyl)-2-fluorophenoxy)ethoxy)ethylamino)-2-((2,6-dioxopiperidin-3-ylamino)methyl)benzoic acid (0.20 g, 0.39 mmol), (5-(2-amino-5-methylthiazol-4-yl)indolin-1-yl)(cyclopropyl)methanone (0.15 g, 0.50 mmol), 2-(7-azabenzotriazol-1-yl)-N,N′,N′-tetramethyluronium hexafluorophosphate (0.37 g, 0.97 mmol) and N,N-diisopropylethylamine (0.20 g, 1.55 mmol) in N,N-dimethylformamide (15 mL) was stirred at room temperature for 18 hrs. The mixture was diluted with water (25 mL), extracted with dichloromethane (30 mL×3), washed with brine (30 mL), dried over anhydrous sodium sulfate, filtered and concentrated in vacuo. The residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15 min) to give N-(4-(1-(cyclopropanecarbonyl)indolin-5-yl)-5-methylthiazol-2-yl)-2-(3-(2-(2-(2-(2,6-dioxopiperidin-3-yl)-1-oxoisoindolin-5-ylamino)ethoxy)ethoxy)-4-fluorophenyl)acetamide (28.0 mg, 9.3% yield) as a white solid. ¹H NMR (400 MHz, DMSO-d₆): δ 12.24 (s, 1H), 10.89 (s, 1H), 8.07 (d, J=6.8 Hz, 1H), 7.49 (s, 1H), 7.42 (d, J=8.4 Hz, 1H), 7.35 (d, J=8.0 Hz, 1H), 7.18-7.13 (m, 2H), 6.90-6.87 (m, 1H), 6.70-6.66 (m, 2H), 6.38 (t, J=5.4 Hz, 1H), 5.01 (dd, J=12.8 Hz, 5.2 Hz, 1H), 4.33 (t, J=7.6 Hz, 2H), 4.25 (d, J=16 Hz, 1H), 4.19 (t, J=4.4 Hz, 2H), 4.11 (d, J=16.8 Hz, 1H), 3.80 (t, J=4.6 Hz, 2H), 3.71 (s, 2H), 3.66 (t, J=5.6 Hz, 2H), 3.29-3.26 (m, 2H), 3.23 (t, J=8.4 Hz, 2H), 2.93-2.84 (m, 1H), 2.59-2.55 (m, 1H), 2.43 (s, 3H), 2.37-2.25 (m, 1H), 2.00-1.90 (m, 2H), 0.90-0.81 (m, 4H). MS (ESI) m/z: 781.1

### Example 30

### Synthesis of 2-(3-(2-(2-((2-(2,6-dioxopiperidin-3-yl)-1,3-dioxoisoindolin-5-yl)amino)ethoxy)ethoxy)-4-fluorophenyl)-N-(5-methyl-4-(1-(1-methyl-1H-imidazole-5-carbonyl)indolin-5-yl)thiazol-2-yl)acetamide (Cpd. No. 29)

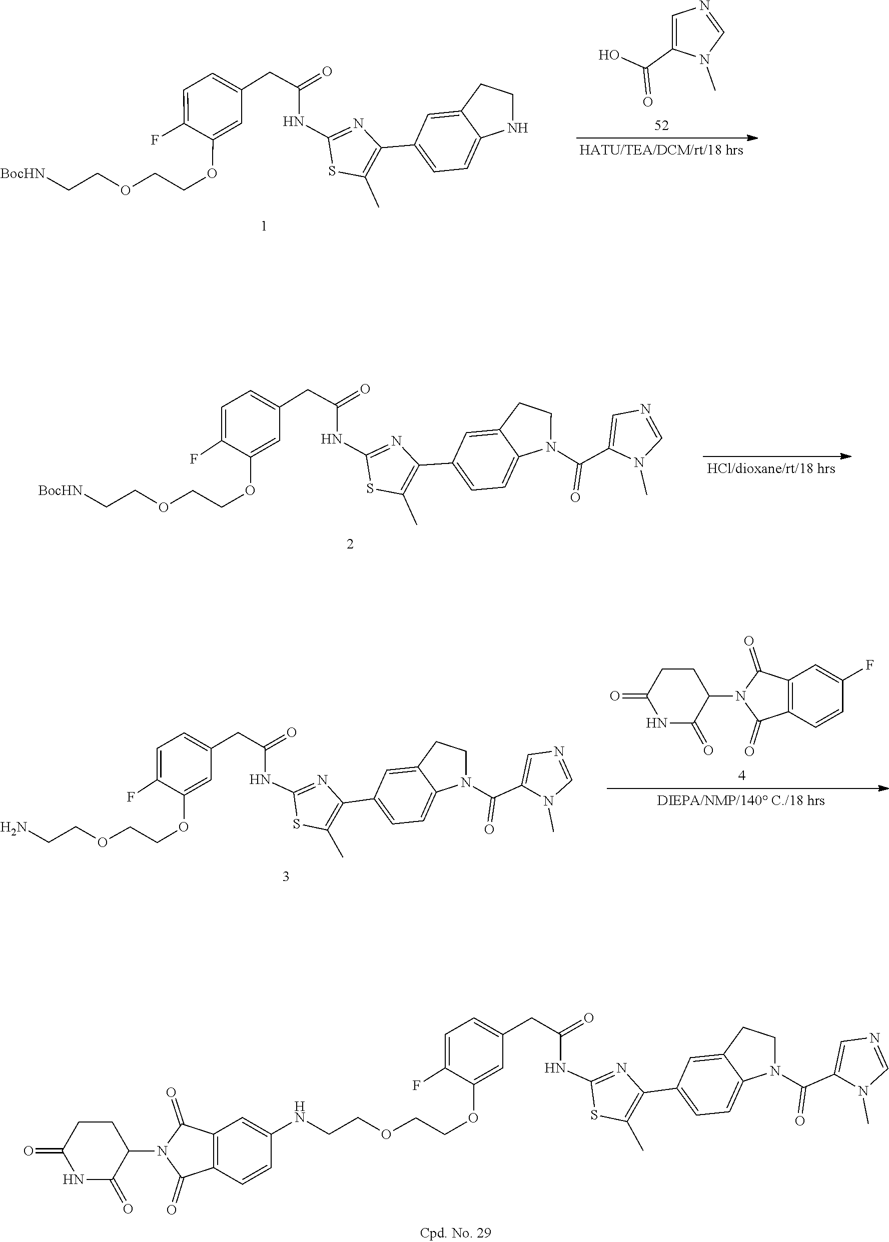

### Step 1. tert-butyl (2-(2-(2-fluoro-5-(2-((5-methyl-4-(1-(1-methyl-1H-imidazole-5-carbonyl)indolin-5-yl)thiazol-2-yl)amino)-2-oxoethyl)phenoxy)ethoxy)ethyl)carbamate

A solution of 1-methyl-1H-imidazole-5-carboxylic acid (0.10 g, 0.79 mmol), tert-butyl (2-(2-(2-fluoro-5-(2-((4-(indolin-5-yl)-5-methylthiazol-2-yl)amino)-2-oxoethyl)phenoxy)ethoxy)ethyl)carbamate (0.45 g, 0.79 mmol), 2-(7-azabenzotriazol-1-yl)-N,N,N′,N′-tetramethyluronium hexafluorophosphate (0.45 g, 1.19 mmol) and triethylamine (0.28 g, 2.78 mmol) in dichloromethane (20 mL) was stirred at room temperature for 18 hrs. The mixture was quenched with water (20 mL), extracted with dichloromethane (100 ml), washed with brine (50 mL), dried over anhydrous sodium sulfate, filtered and concentrated in vacuo. The residue was purified by flash column chromatography (dichloromethane/methanol=3/97) to give crude tert-butyl (2-(2-(2-fluoro-5-(2-((5-methyl-4-(1-(1-methyl-1H-imidazole-5-carbonyl)indolin-5-yl)thiazol-2-yl)amino)-2-oxoethyl)phenoxy)ethoxy)ethyl)carbamate (0.50 g, 93% yield) as an off-white solid. MS (ESI) m/z 679.2 [M+H]⁺

### Step 2. 2-(3-(2-(2-aminoethoxy)ethoxy)-4-fluorophenyl)-N-(5-methyl-4-(1-(1-methyl-1H-imidazole-5-carbonyl)indolin-5-yl)thiazol-2-yl)acetamide

A solution of tert-butyl (2-(2-(2-fluoro-5-(2-((5-methyl-4-(1-(1-methyl-1H-imidazole-5-carbonyl)indolin-5-yl)thiazol-2-yl)amino)-2-oxoethyl)phenoxy)ethoxy)ethyl)carbamate (0.50 g, 0.74 mmol) and hydrochloric acid (4 M in 1,4-dioxane, 10 mL) in 1,4-dioxane (2 mL) was stirred at room temperature for 18 hrs. The solvent was removed in vacuo and the residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15.0 min) to give 2-(3-(2-(2-aminoethoxy)ethoxy)-4-fluorophenyl)-N-(5-methyl-4-(1-(1-methyl-1H-imidazole-5-carbonyl)indolin-5-yl)thiazol-2-yl)acetamide (0.25 g, 58% yield) as a yellow solid. MS (ESI) m/z 579.2 [M+H]⁺

### Step 3. 2-(3-(2-(2-((2-(2,6-dioxopiperidin-3-yl)-1,3-dioxoisoindolin-5-yl)amino)ethoxy)ethoxy)-4-fluorophenyl)-N-(5-methyl-4-(1-(1-methyl-1H-imidazole-5-carbonyl)indolin-5-yl)thiazol-2-yl)acetamide

A solution of 2-(3-(2-(2-aminoethoxy)ethoxy)-4-fluorophenyl)-N-(5-methyl-4-(1-(1-methyl-1H-imidazole-5-carbonyl)indolin-5-yl)thiazol-2-yl)acetamide (0.12 g, 0.21 mmol), 2-(2,6-dioxopiperidin-3-yl)-5-fluoroisoindoline-1,3-dione (69.0 mg, 0.25 mmol) and N,N-diisopropylethylamine (0.11 g, 0.83 mmol) in 1-methylpyrrolidin-2-one (2 mL) was stirred at 140° C. for 18 hrs. The mixture was cooled to room temperature, diluted with water (10 mL), extracted with ethyl acetate (20 mL×3), dried over anhydrous sodium sulfate, filtered and concentrated in vacuo. The residue was purified by prep-HPLC (Column: Welch Xtimate 21.2*250 mm C18, 10 μm. Mobile Phase: A: water (10 mmol/L ammonium bicarbonate) B: acetonitrile; B %: 30%-70% in 15.0 min) to give 2-(3-(2-(2-((2-(2,6-dioxopiperidin-3-yl)-1,3-dioxoisoindolin-5-yl)amino)ethoxy)ethoxy)-4-fluorophenyl)-N-(5-methyl-4-(1-(1-methyl-1H-imidazole-5-carbonyl)indolin-5-yl)thiazol-2-yl)acetamide (8.5 mg, 4.9% yield) as a yellow solid. ¹H NMR (400 MHz, DMSO-d₆): δ 12.29 (brs, 1H), 11.06 (s, 1H), 8.07 (d, J=8.8 Hz, 1H), 7.84 (s, 1H), 7.59 (s, 1H), 7.57-7.52 (m, 2H), 7.48 (d, J=8.4 Hz, 1H), 7.17-7.13 (m, 3H), 6.99 (m, 1H), 6.89-6.87 (m, 2H), 5.04-5.00 (m, 1H), 4.36 (t, J=8.4 Hz, 2H), 4.19 (t, J=3.6 Hz, 2H), 3.87-3.76 (m, 5H), 3.71 (s, 2H), 3.66 (d, J=5.6 Hz, 2H), 3.44-3.35 (m, 2H), 3.19 (t, J=8.4 Hz, 2H), 2.91-2.82 (m, 1H), 2.61-2.54 (m, 2H), 2.45 (s, 3H), 2.01-1.97 (m, 1H). MS (ESI) m/z: 835.2 [M+H]⁺.

### Example 31

### Synthesis of 2-(3-(2-(2-((2-(2,6-dioxopiperidin-3-yl)-1-oxoisoindolin-5-yl)amino)ethoxy)ethoxy)-4-fluorophenyl)-N-(5-methyl-4-(1-(1-methyl-1H-imidazole-5-carbonyl)indolin-5-yl)thiazol-2-yl)acetamide (Cpd. No. 30)

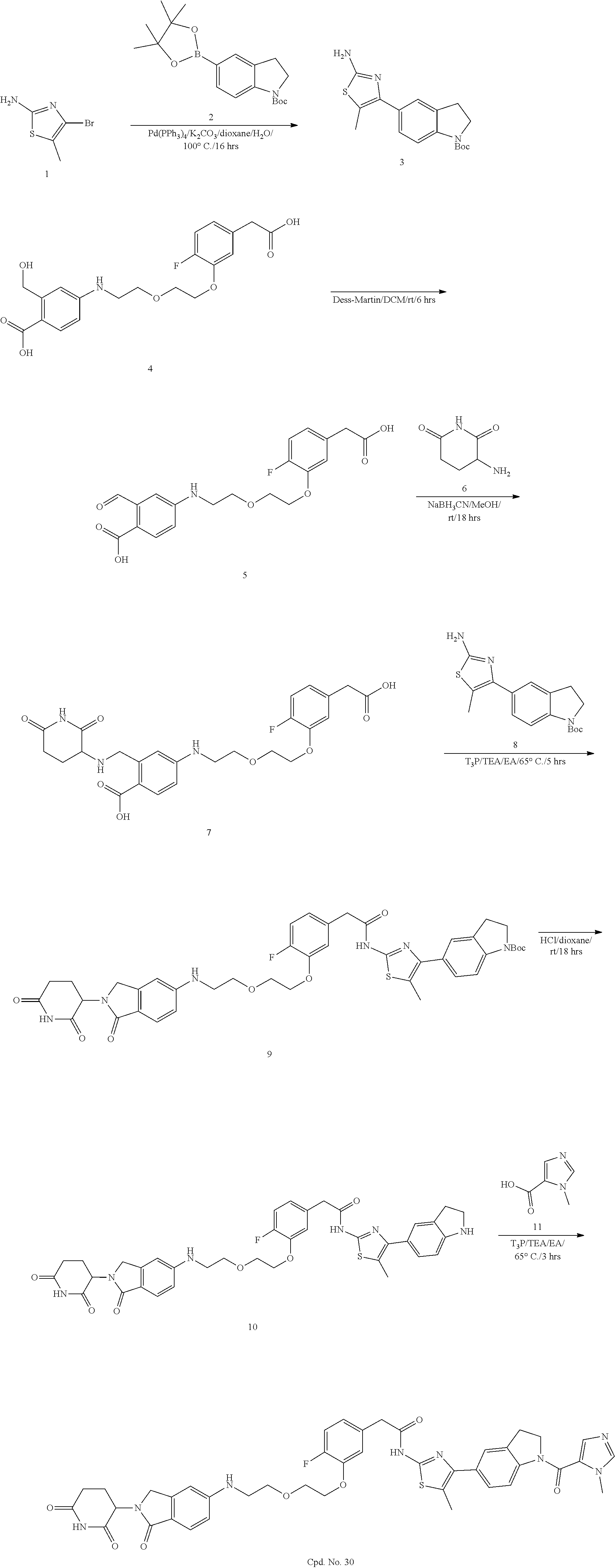

### Step 1. tert-butyl 5-(2-amino-5-methylthiazol-4-yl)indoline-1-carboxylate

A solution of 4-bromo-5-methylthiazol-2-anine (0.28 g, 1.5 mmol), tert-butyl 5-(4,4,5,5-tetramethyl-1,3,2-dioxaborolan-2-yl)indoline-1-carboxylate (0.50 g, 1.5 mmol), tetrakis(triphenylphosphine)palladium (0.17 r, 0.14 mmol) and potassium carbonate (0.60 g, 4.34 mmol) in 1,4-dioxane (20 mL) and water (4 mL) was stirred at 100° C. for 16 hrs under nitrogen atmosphere. The mixture was cooled to room temperature, diluted with water (20 mL), extracted with dichloromethane (25 mL×3), dried over anhydrous sodium sulfate, filtered and concentrated in vacuo. The residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15 min) to give tert-butyl 5-(2-amino-5-methylthiazol-4-yl)indoline-1-carboxylate (0.20 g, 42% yield) as a white solid. MS (ESI) m/z: 332.0 [M+H]⁺.

### Step 2. 4-(2-(2-(5-(carboxymethyl)-2-fluorophenoxy)ethoxy)ethylamino)-2-formylbenzoic acid

A solution of 4-(2-(2-(5-(carboxymethyl)-2-fluorophenoxy)ethoxy)ethylamino)-2-(hydroxymethyl)benzoic acid (1.50 g, 3.68 mmol) and Dess-martin reagent (2.34 g, 5.52 mmol) in dichloromethane (20 mL) was stirred at room temperature for 18 hrs. The mixture was diluted with water (20 mL), extracted with dichloromethane (30 mL×3), dried over anhydrous sodium sulfate, filtered and concentrated in vacuo. The residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15 min) to give 4-(2-(2-(5-(carboxymethyl)-2-fluorophenoxy)ethoxy)ethylamino)-2-formylbenzoic acid (0.70 g, 47% yield) as a white solid. MS (ESI) m/z: 406.2 [M+H]⁺.

### Step 3. 4-(2-(2-(5-(carboxymethyl)-2-fluorophenoxy)ethoxy)ethylamino)-2-((2,6-dioxopiperidin-3-ylamino)methyl)benzoic acid

To a solution of 4-(2-(2-(5-(carboxymethyl)-2-fluorophenoxy)ethoxy)ethylamino)-2-formylbenzoic acid (0.68 g, 1.68 mmol) and 3-aminopiperidine-2,6-dione hydrochloride (0.41 g, 2.5 mmol) in methanol (30 mL) was added sodium acetate (0.55 g, 6.71 mmol) and sodium cyanoborohydride (0.32 g, 5.03 mmol) at room temperature. After addition, the mixture was stirred at room temperature for 18 hrs. The mixture was diluted with water (20 mL), extracted with dichloromethane (30 mL×3), dried over anhydrous sodium sulfate, filtered and concentrated in vacuo. The residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15 min) to give 4-(2-(2-(5-(carboxymethyl)-2-fluorophenoxy)ethoxy)ethylamino)-2-((2,6-dioxopiperidin-3-ylamino)methyl)benzoic acid (0.41 g, 47.% yield) as a white solid. MS (ESI) m/z: 517.9 [M+H]⁺.

### Step 4. tert-butyl 5-(2-(2-(3-(2-(2-((2-(2,6-dioxopiperidin-3-yl)-1-oxoisoindolin-5-yl)amino)ethoxy)ethoxy)-4-fluorophenyl)acetamido)-5-methylthiazol-4-yl)indoline-1-carboxylate

A solution of 4-(2-(2-(5-(carboxymethyl)-2-fluorophenoxy)ethoxy)ethylamino)-2-((2,6-dioxopiperidin-3-ylamino)methyl)benzoic acid (0.34 g, 0.66 mmol), tert-butyl 5-(2-amino-5-methylthiazol-4-yl)indoline-1-carboxylate (0.26 g, 0.78 mmol), propylphosphonic anhydride solution (50% (wt %) in ethyl acetate, 2.09 g, 3.28 mmol) and triethylamine (0.33 g, 3.28 mmol) in ethyl acetate (12 mL) was stirred at 65° C. for 5 hrs. The mixture was cooled to room temperature, diluted with water (20 mL), extracted with dichloromethane (30 mL×3), washed with brine (30 mL), dried over anhydrous sodium sulfate, filtered and concentrated in vacuo. The residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15 min) to give tert-butyl 5-(2-(2-(3-(2-(2-((2-(2,6-dioxopiperidin-3-yl)-1-oxoisoindolin-5-yl)amino)ethoxy)ethoxy)-4-fluorophenyl)acetamido)-5-methylthiazol-4-yl)indoline-1-carboxylate (0.20 g, 37% yield) as a white solid. MS (ESI) m/z: 813.1 [M+H]⁺.

### Step 5. 2-(3-(2-(2-((2-(2,6-dioxopiperidin-3-yl)-1-oxoisoindolin-5-yl)amino) ethoxy)ethoxy)-4-fluorophenyl)-N-(4-(indolin-5-yl)-5-methylthiazol-2-yl)acetamide

A solution of tert-butyl 5-(2-(2-(3-(2-(2-((2-(2,6-dioxopiperidin-3-yl)-1-oxoisoindolin-5-yl)amino)ethoxy)ethoxy)-4-fluorophenyl)acetamido)-5-methylthiazol-4-yl)indoline-1-carboxylate (0.19 g, 0.23 mmol) in hydrochloric acid (2 M in 1,4-dioxane, 15 mL) and 1,4-dioxane (10 mL) was stirred at room temperature for 18 hrs. The mixture was concentrated in vacuo. The residue was dissolved in dichloromethane (100 mL), washed with saturated sodium bicarbonate aqueous solution, dried over anhydrous sodium sulfate, filtered and concentrated in vacuo to give 2-(3-(2-(2-((2-(2,6-dioxopiperidin-3-yl)-1-oxoisoindolin-5-yl)amino)ethoxy)ethoxy)-4-fluorophenyl)-N-(4-(indolin-5-yl)-5-methylthiazol-2-yl)acetamide (0.16 g, 96% yield) as a white solid. MS (ESI) m/z: 713.3 [M+H]⁺.

### Step 6. 2-(3-(2-(2-((2-(2,6-dioxopiperidin-3-yl)-1-oxoisoindolin-5-yl)amino) ethoxy)ethoxy)-4-fluorophenyl)-N-(5-methyl-4-(1-(1-methyl-1H-imidazole-5-carbonyl)indolin-5-yl)thiazol-2-yl)acetamide

A solution of 2-(3-(2-(2-((2-(2,6-dioxopiperidin-3-yl)-1-oxoisoindolin-5-yl)amino)ethoxy)ethoxy)-4-fluorophenyl)-N-(4-(indolin-5-yl)-5-methylthiazol-2-yl)acetamide (0.15 g, 0.21 mmol), 1-methyl-1H-imidazole-5-carboxylic acid (29.2 mg, 0.23 mmol), propylphosphonic anhydride solution (50% (wt %) in ethyl acetate, 0.67 g, 1.05 mmol) and triethylamine (0.11 g, 1.05 mmol) in ethyl acetate (10 mL) was stirred at 65° C. for 3 hrs. The mixture was cooled to room temperature, diluted with water (20 mL), extracted with dichloromethane (30 mL×3), washed with brine (20 mL), dried over anhydrous sodium sulfate, filtered and concentrated in vacuo. The residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15 min) to give 2-(3-(2-(2-((2-(2,6-dioxopiperidin-3-yl)-1-oxoisoindolin-5-yl)amino)ethoxy) ethoxy)-4-fluorophenyl)-N-(5-methyl-4-(1-(1-methyl-1H-imidazole-5-carbonyl)indolin-5-yl)thiazol-2-yl)acetamide (11.4 mg, 6.6% yield) as a white solid. ¹H NMR (400 MHz, DMSO-d₆): δ 12.31 (brs, 1H), 10.93 (s, 1H), 8.08 (d, J=8.0 Hz, 1H), 7.85 (s, 1H), 7.60 (s, 1H), 7.54 (s, 1H), 7.49 (d, J=8.4 Hz, 1H), 7.37 (d, J=8.4 Hz, 1H), 7.19-7.14 (m, 2H), 6.91-6.88 (m, 1H), 6.70-6.66 (m, 2H), 6.40 (t, J=5.4 Hz, 1H), 5.01 (dd, J=13.2 Hz, 5.2 Hz, 1H), 4.37 (t, J=8.4 Hz, 2H), 4.24 (d, J=16.8 Hz, 1H), 4.19 (t, J=4.6 Hz, 2H), 4.11 (d, J=16.8 Hz, 1H) 3.82 (m, 2H) 3.80 (s, 3H), 3.72 (s, 2H), 3.66 (t, J=5.6 Hz, 2H), 3.29 (m, 2H), 3.19 (t, J=8.4 Hz, 2H), 2.93-2.84 (m, 1H), 2.68-2.54 (m, 1H), 2.45 (s, 3H), 2.37-2.26 (m, 1H), 1.95-1.89 (m, 1H). MS (ESI) m/z: 821.0 [M+H]⁺.

2-(3-(2-(2-((2-(2,6-dioxopiperidin-3-yl)-1-oxoisoindolin-5-yl)amino)ethoxy)ethoxy)-4-fluorophenyl)-N-(5-methyl-4-(1-(1-methyl-1H-imidazole-5-carbonyl)indolin-5-yl)thiazol-2-yl)acetamide (Cpd. No. 30) can also be prepared according to the following alternative route:

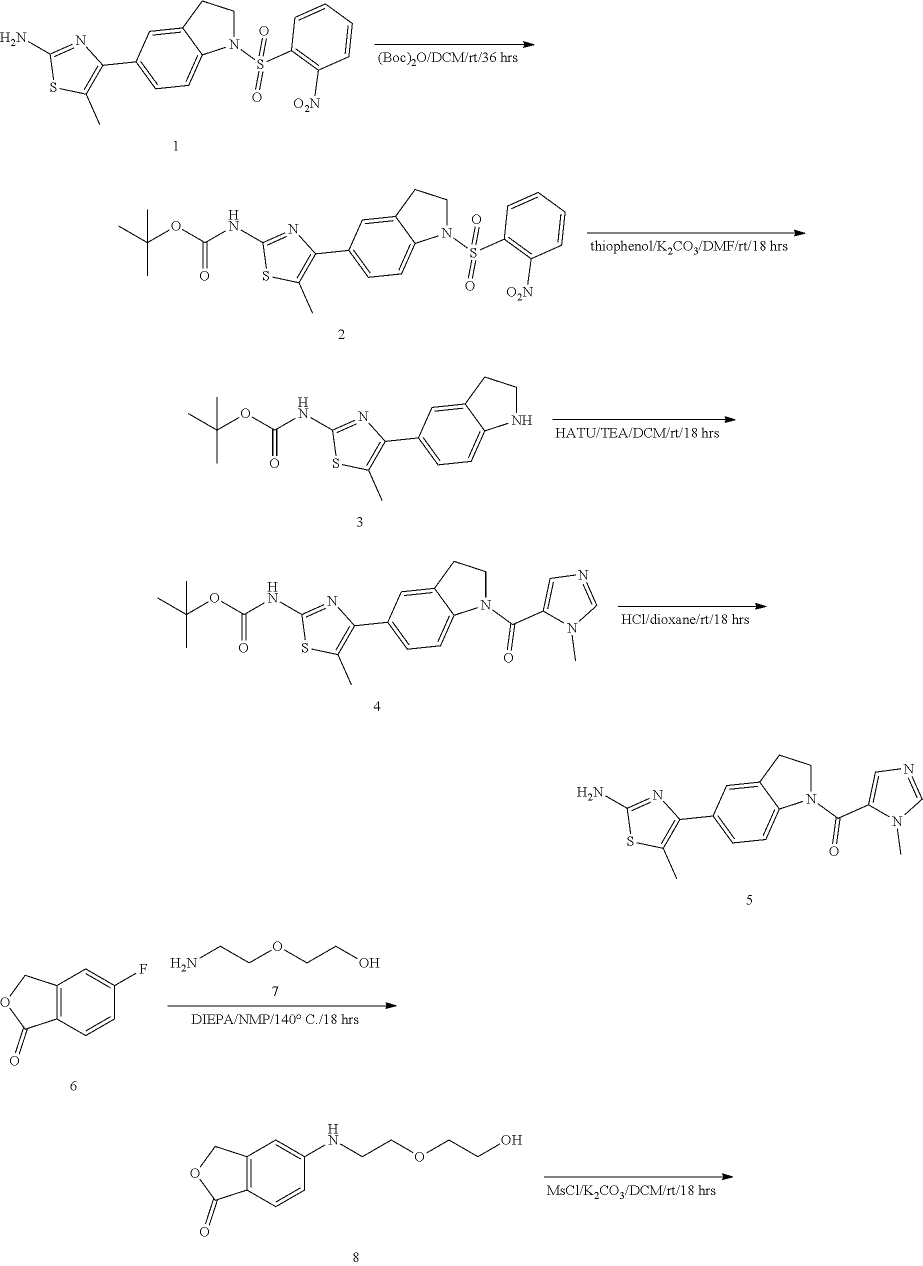

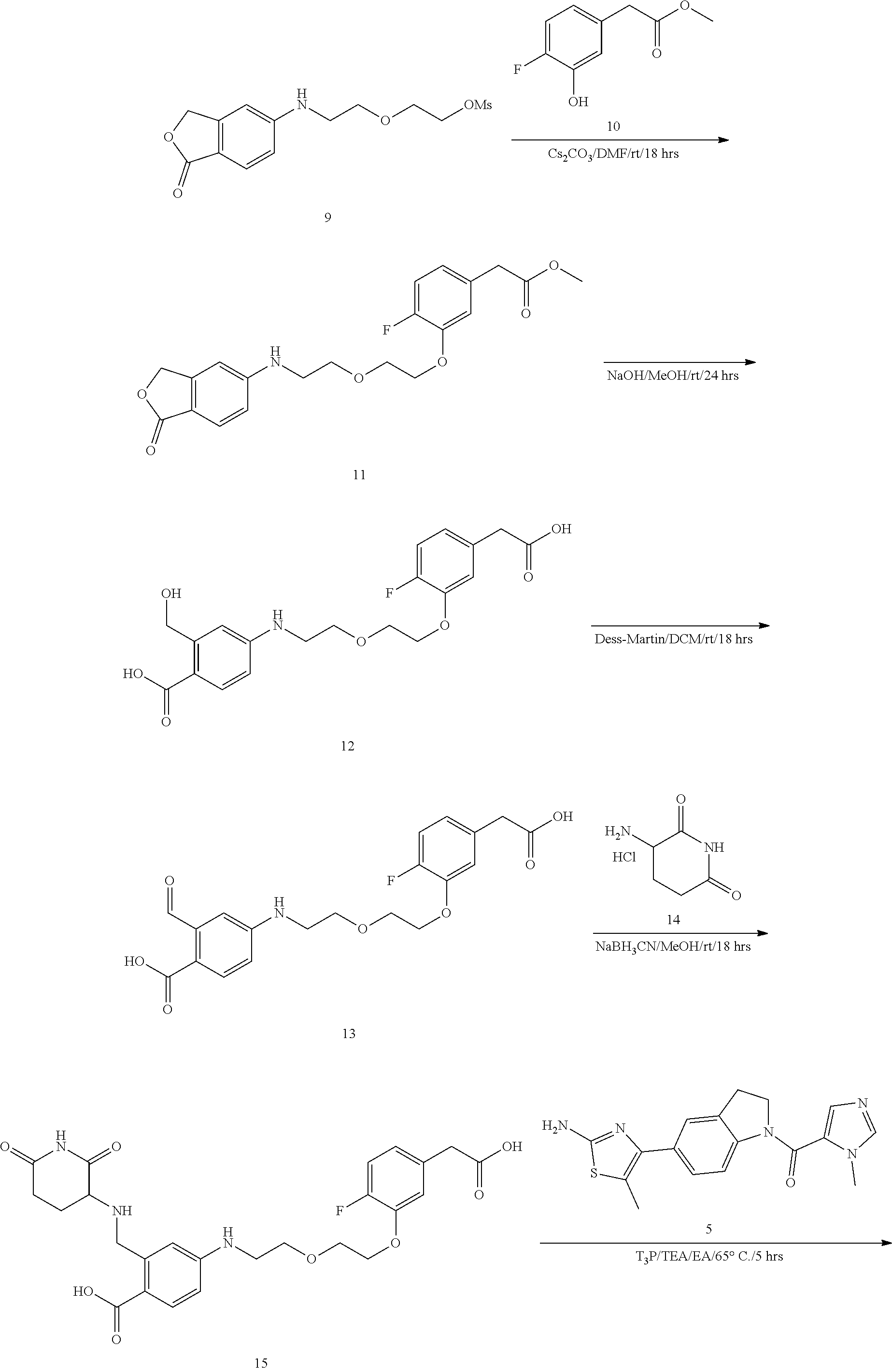

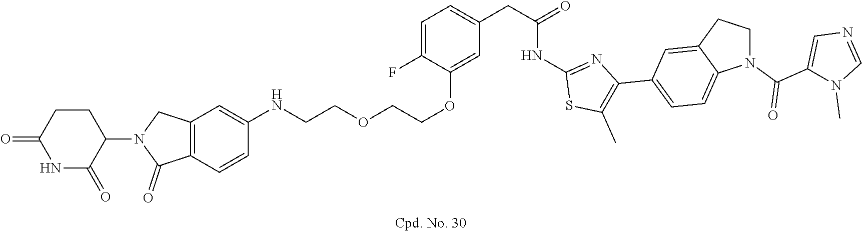 [SMILES: Cc1sc(NC(=O)Cc2ccc(F)c(OCCOCCNc3ccc4c(c3)CN(C3CCC(=O)NC3=O)C4=O)c2)nc1-c1ccc2c(c1)CCN2C(=O)c1cncn1C]

### Step 1. tert-butyl 5-methyl-4-(1-(2-nitrophenylsulfonyl)indolin-5-yl)thiazol-2-ylcarbamate

A solution of 5-methyl-4-(1-(2-nitrophenylsulfonyl)indolin-5-yl)thiazol-2-amine (5.00 g, 12.0 mmol), di-tert-butyl dicarbonate (7.86 g, 36.0 mmol), N,N-diisopropylethylamine (7.76 g, 60.0 mmol) and N,N-dimethylpyridin-4-amine (0.01 g, 0.12 mmol) in dichloromethane (100 mL) was stirred at room temperature for 36 hrs. Dichloromethane was removed in vacuo. The residue was suspended in ether (100 mL) and filtered. The filter cake was washed with ether (30 mL×2) and dried to give tert-butyl 5-methyl-4-(1-(2-nitrophenylsulfonyl)indolin-5-yl)thiazol-2-ylcarbamate (2.50 g, 40.3% yield) as a gray solid. MS (ESI) m/z: 517.1 [M+H]⁺.

### Step 2. tert-butyl 4-(indolin-5-yl)-5-methylthiazol-2-ylcarbamate

A solution of tert-butyl 5-methyl-4-(1-(2-nitrophenylsulfonyl)indolin-5-yl)thiazol-2-ylcarbamate (2.40 g, 4.65 mmol), potassium carbonate (0.71 g, 5.1 mmol) and thiophenol (1.02 g, 9.29 mmol) in N,N-dimethylformamide (30 mL) was stirred at room temperature for 18 hrs. The mixture was diluted with water (50 mL), extracted with dichloromethane (50 mL×2), washed with brine (30 mL), dried over anhydrous sodium sulfate, filtered and concentrated in vacuo. The residue was purified by flash column chromatography (dichloromethane/methanol=10/1) to give tert-butyl 4-(indolin-5-yl)-5-methylthiazol-2-ylcarbamate (0.70 g, 45% yield) as a yellow solid. MS (ESI) m/z: 332.1 [M+H]⁺.

### Step 3. tert-butyl 5-methyl-4-(1-(1-methyl-1H-imidazole-5-carbonyl)indolin-5-yl)thiazol-2-ylcarbamate

A solution of tert-butyl 4-(indolin-5-yl)-5-methylthiazol-2-ylcarbamate (0.68 g, 2.05 mmol), 1-methyl-1H-imidazole-5-carboxylic acid (0.52 g, 4.1 mmol), 2-(7-azabenzotriazol-1-yl)-N,N,N′,N′-tetramethyluronium hexafluorophosphate (1.56 g, 4.10 mmol) and triethylamine (0.83 g, 8.2 mmol) in N,N-dimethylfomamide (20 mL) was stirred at room temperature for 18 hrs. The mixture was diluted with water (50 mL), extracted with dichloromethane (50 mL×2), dried over anhydrous sodium sulfate, filtered and concentrated in vacuo. The residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15 min) to give tert-butyl 5-methyl-4-(1-(1-methyl-1H-imidazole-5-carbonyl)indolin-5-yl)thiazol-2-ylcarbamate (0.57 g, 63% yield) as a white solid. MS (ESI) m/z: 440.2 [M+H]⁺.

### Step 4. (5-(2-amino-5-methylthiazol-4-yl)indolin-1-yl)(1-methyl-1H-imidazol-5-yl)methanone

A solution of tert-butyl 5-methyl-4-(1-(1-methyl-1H-imidazole-5-carbonyl) indolin-5-yl)thiazol-2-ylcarbamate (0.55 g, 1.25 mmol) in hydrochloric acid (2 M in dioxane, 20 mL) was stirred at room temperature for 18 hrs. The solvent was removed in vacuo. The residue was treated with saturated sodium carbonate aqueous solution and filtered. The filter cake was washed with water (10 mL×3) and dried to give (5-(2-amino-5-methylthiazol-4-yl) indolin-1-yl)(1-methyl-1H-imidazol-5-yl)methanone (0.23 g, 54% yield) as a yellow solid. MS (ESI) m/z: 340.1 [M+H]⁺.

### Step 5. 5-(2-(2-hydroxyethoxy)ethylamino)isobenzofuran-1(3H)-one

A solution of 5-fluoroisobenzofuran-1(3H)-one (10.00 g, 65.74 mmol), 2-(2-aminoethoxy)ethanol (10.37 g, 98.61 mmol) and N,N-diisopropylethylamine (25.49 g, 197.2 mmol) in 1-methylpyrrolidin-2-one (100 mL) was stirred at 140° C. for 18 hrs. The mixture was cooled to room temperature, diluted with water (100 mL), extracted with dichloromethane (200 mL×8), dried over anhydrous sodium sulfate, filtered and concentrated in vacuo. The residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15 min) to give 5-(2-(2-hydroxyethoxy)ethylamino) isobenzofuran-1(3H)-one (13.00 g, 83.4% yield) as a white solid. MS (ESI) m/z: 238.1 [M+H]⁺.

### Step 6. 2-(2-(1-oxo-1,3-dihydroisobenzofuran-5-ylamino)ethoxy)ethyl methanesulfonate

To a solution of 5-(2-(2-hydroxyethoxy)ethylamino)isobenzofuran-1(3H)-one (10.98 g, 46.28 mmol) and potassium carbonate (25.58 g, 185.1 mmol) in dichloromethane (100 mL) was added methanesulfonyl chloride (6.36 g, 55.53 mmol) at 0° C. After addition, the mixture was stirred at room temperature for 18 hrs. The mixture was diluted with water (150 mL), extracted with dichloromethane (100 mL×3), washed with brine (80 mL), dried over anhydrous sodium sulfate, filtered and concentrated in vacuo to give 2-(2-(1-oxo-1,3-dihydroisobenzofuran-5-ylamino)ethoxy)ethyl methanesulfonate (12.50 g, 85.7% yield) as an off-white solid. MS (ESI) m/z: 316.1 [M+H]⁺.

### Step 7. methyl 2-(4-fluoro-3-(2-(2-(1-oxo-1,3-dihydroisobenzofuran-5-ylamino) ethoxy)ethoxy) phenyl)acetate

A solution of 2-(2-(1-oxo-1,3-dihydroisobenzofuran-5-ylamino)ethoxy)ethyl methanesulfonate (8.00 g, 25.4 mmol), methyl 2-(4-fluoro-3-hydroxyphenyl)acetate (4.67 g, 25.4 mmol) and cesium carbonate (33.06 g, 101.5 mmol) in N,N-dimethylformamide (90 mL) was stirred at room temperature for 18 hrs. The mixture was diluted with water (120 mL), extracted with dichloromethane (100 mL×3), washed with brine (50 mL), dried over anhydrous sodium sulfate, filtered and concentrated in vacuo to give crude methyl 2-(4-fluoro-3-(2-(2-(1-oxo-1,3-dihydroisobenzofuran-5-ylamino)ethoxy)ethoxy) phenyl)acetate (9.30 g, 90.9% yield) as a yellow solid. MS (ESI) m/z: 404.1 [M+H]⁺.

### Step 8. 4-(2-(2-(5-(carboxymethyl)-2-fluorophenoxy)ethoxy)ethylamino)-2-(hydroxymethyl) benzoic acid

A solution of methyl 2-(4-fluoro-3-(2-(2-(1-oxo-1,3-dihydroisobenzofuran-5-ylamino)ethoxy)ethoxy)phenyl) acetate (9.20 g, 22.8 mmol) and sodium hydroxide (4.56 g, 114 mmol) in methanol (50 mL) and water (50 mL) was stirred at room temperature for 24 hrs. The mixture was diluted with water (50 mL), acidified with aqueous hydrochloric acid (2 M) solution to pH=3, extracted with dichloromethane (100 mL×5), dried over anhydrous sodium sulfate, filtered and concentrated in vacuo. The residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15 min) to give 4-(2-(2-(5-(carboxymethyl)-2-fluorophenoxy)ethoxy)ethylamino)-2-(hydroxymethyl)benzoic acid (6.80 g, 73.2% yield) as a white solid. MS (ESI) m/z: 408.1 [M+H]⁺.

### Step 9. 4-(2-(2-(5-(carboxymethyl)-2-fluorophenoxy)ethoxy)ethylamino)-2-formylbenzoic acid

A solution of 4-(2-(2-(5-(carboxymethyl)-2-fluorophenoxy)ethoxy)ethylamino)-2-(hydroxymethyl)benzoic acid (6.70 g, 16.5 mmol) and Dess-martin reagent (10.46 g, 24.67 mmol) in dichloromethane (120 mL) was stirred at room temperature for 18 brs. The mixture was diluted with water (60 mL), extracted with dichloromethane (100 mL×3), dried over anhydrous sodium sulfate, filtered and concentrated in vacuo. The residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15 min) to give 4-(2-(2-(5-(carboxymethyl)-2-fluorophenoxy)ethoxy)ethylamino)-2-formylbenzoic acid (4.30 g, 64.4% yield) as a white solid. MS (ESI) m/z: 406.1 [M+H]⁺.

### Step 10. 4-(2-(2-(5-(carboxymethyl)-2-fluorophenoxy)ethoxy)ethylamino)-2-((2,6-dioxopiperidin-3-ylamino)methyl)benzoic acid

To a solution of 4-(2-(2-(5-(carboxymethyl)-2-fluorophenoxy)ethoxy)ethylamino)-2-formylbenzoic acid (4.28 g, 10.6 mmol) and 3-aminopiperidine-2,6-dione hydrochloride (2.61 g, 15.8 mmol) in methanol (100 mL) was added sodium acetate (3.46 g, 42.2 mmol) and sodium borohydride (1.99 g, 31.7 mmol) at room temperature. After addition, the mixture was stirred at room temperature for 18 hrs. The mixture was diluted with water (100 mL), extracted with dichloromethane (100 mL×5), dried over anhydrous sodium sulfate, filtered and concentrated in vacuo. The residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15 min) to give 4-(2-(2-(5-(carboxymethyl)-2-fluorophenoxy)ethoxy)ethylamino)-2-((2,6-dioxopiperidin-3-ylamino)methyl)benzoic acid (2.80 g, 51.2% yield) as a white solid. MS (ESI) m/z: 518.2 [M+H]⁺.

### Step 11, 2-(3-(2-(2-((2-(2,6-dioxopiperidin-3-yl)-1-oxoisoindolin-5-yl)amino)ethoxy)ethoxy)-4-fluorophenyl)-N-(5-methyl-4-(1-(1-methyl-1H-imidazole-5-carbonyl)indolin-5-yl)thiazol-2-yl)acetamide

A solution of 4-(2-(2-(5-(carboxymethyl)-2-fluorophenoxy)ethoxy)ethylamino)-2-((2,6-dioxopiperidin-3-ylamino) methyl)benzoic acid (0.60 g, 1.16 mmol), (5-(2-amino-5-methylthiazol-4-yl)indolin-1-yl)(1-methyl-1H-imidazol-5-yl)methanone (0.39 g, 1.16 mmol), propylphosphonic anhydride solution (50% (wt %) in ethyl acetate, 3.69 g, 5.80 mmol) and triethylamine (0.59 g, 5.8 mmol) in ethyl acetate (25 mL) was stirred at 65° C. for 5 hrs. The mixture was cooled to room temperature, diluted with water (30 mL), extracted with dichloromethane (50 mL×3), washed with brine (30 mL), dried over anhydrous sodium sulfate, filtered and concentrated in vacuo. The residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15 min) to give 2-(3-(2-(2-((2-(2,6-dioxopiperidin-3-yl)-1-oxoisoindolin-5-yl)amino)ethoxy) ethoxy)-4-fluorophenyl)-N-(5-methyl-4-(1-(1-methyl-1H-imidazole-5-carbonyl)indolin-5-yl)thiazol-2-yl)acetamide (62.0 mg, 6.5% yield) as a white solid. ¹H NMR (400 MHz, DMSO-d₆): δ 12.30 (brs, 1H), 10.93 (s, 1H), 8.08 (d, J=8.0 Hz, 1H), 7.85 (s, 1H), 7.59 (s, 1H), 7.54 (s, 1H), 7.49 (d, J=8.4 Hz, 1H), 7.37 (d, J=8.4 Hz, 1H), 7.19-7.14 (m, 2H), 6.90-6.87 (m, 1H), 6.69-6.65 (m, 2H), 6.40 (t, J=5.2 Hz, 1H), 5.00 (dd, J=13.2 Hz, 5.2 Hz, 1H), 4.36 (t, J=8.4 Hz, 1H), 4.25-4.09 (m, 3.89-3.78 (m, 5H), 3.71 (s, 2H), 3.65 (t, J=5.6 Hz, 2H), 3.28 (q, J=5.6 Hz, 2H), 3.19 (t, J=8.4 Hz, 2H), 2.93-2.84 (m, 1H), 2.67-2.54 (m, 1H), 2.45 (s, 3H), 2.36-2.25 (m, 1H), 1.93-1.89 (m, 1H). MS (ESI) m/z: 821.0 [M+H]⁺.

### Example 32

### Synthesis of 2-(3-(2-(4-(2-(2,6-dioxopiperidin-3-yl)-1,3-dioxoisoindolin-4-yl)piperazin-1-yl)ethoxy)phenyl)-N-(5-methyl-4-(1-(2-methylbenzoyl)indolin-5-yl)thiazol-2-yl)acetamide (Cpd. No. 31)

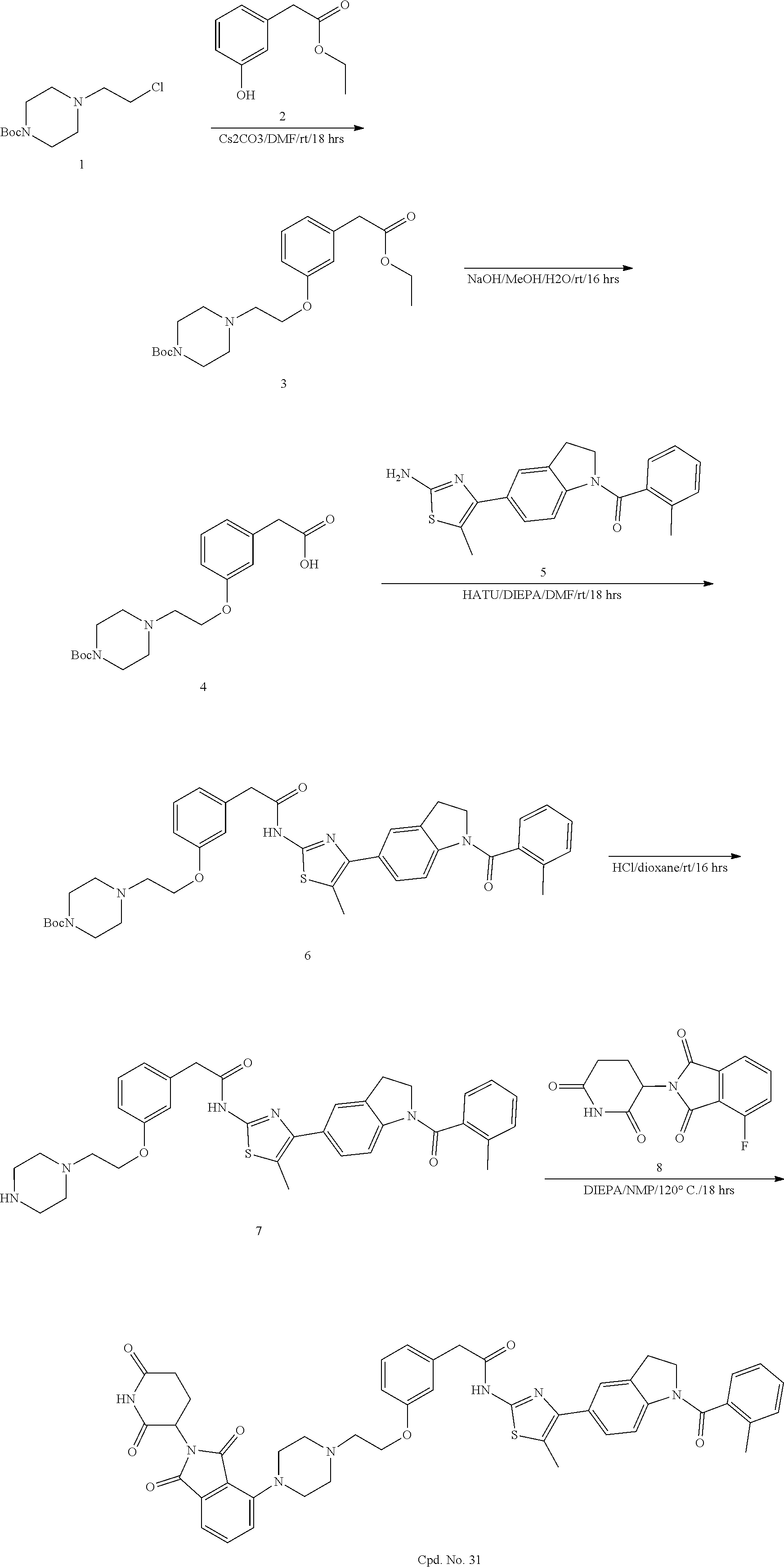

### Step 1. tert-butyl 4-(2-(3-(2-ethoxy-2-oxoethyl)phenoxy)ethyl)piperazine-1-carboxylate

A solution of ethyl 2-(3-hydroxyphenyl)acetate (0.29 g, 1.61 mmol), tert-butyl 4-(2-chloroethyl)piperazine-1-carboxylate (0.40 g, 1.61 mmol) and cesium carbonate (1.57 g, 4.83 mmol) in N, N-dimethylformamide (20 mL) was stirred at room temperature for 18 hrs. The mixture was diluted with water (40 mL), extracted with ethyl acetate (50 mL×2), washed with brine (20 mL×3), dried over anhydrous sodium sulfate, filtered and concentrated in vacuo to give tert-butyl 4-(2-(3-(2-ethoxy-2-oxoethyl)phenoxy)ethyl)piperazine-1-carboxylate (0.36 g, 57% yield) as off-white solid. MS (ESI) m/z 393.2 [M+H]⁺.

### Step 2. 2-(3-(2-(4-(tert-butoxycarbonyl)piperazin-1-yl)ethoxy)phenyl)acetic acid

A solution of tert-butyl 4-(2-(3-(2-ethoxy-2-oxoethyl)phenoxy)ethyl)piperazine-1-carboxylate (0.36 g, 0.92 mmol), sodium hydroxide (0.11 g, 2.75 mmol) in methanol (20 mL) and water (20 mL) was stirred at room temperature for 16 hrs. The mixture was acidified with aqueous hydrochloric acid solution (2 M) to pH=3, extracted with dichloromethane (30 mL×4), dried over anhydrous sodium sulfate, filtered and concentrated in vacuo. The residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15.0 min) to give 2-(3-(2-(4-(tert-butoxycarbonyl)piperazin-1-yl)ethoxy)phenyl) acetic acid (0.26 g, 78% yield) as a white solid. MS (ESI) m/z: 365.2 [M+H]⁺.

### Step 3. tert-butyl 4-(2-(3-(2-(5-methyl-4-(1-(2-methylbenzoyl)indolin-5-yl)thiazol-2-ylamino)-2-oxoethyl)phenoxy)ethyl)piperazine-1-carboxylate

A solution of 2-(3-(2-(4-(tert-butoxycarbonyl)piperazin-1-yl)ethoxy)phenyl)acetic acid (0.24 g, 0.66 mmol), (5-(2-amino-5-methylthiazol-4-yl)indolin-1-yl)(o-tolyl)methanone (0.35 g, 0.99 mmol), 2-(7-azabenzotriazol-1-yl)-N,N,N′,N′-tetramethyluronium hexafluorophosphate (0.38 g, 0.99 mmol) and N,N-diisopropylethylamine (0.34 g, 2.63 mmol) in N,N-dimethylformamide (10 mL) was stirred at room temperature for 18 hrs. The mixture was diluted with water (30 mL), extracted with dichloromethane (40 mL×2), washed with brine (20 mL), dried over anhydrous sodium sulfate, filtered and concentrated in vacuo. The residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15.0 min) to give tert-butyl 4-(2-(3-(2-(5-methyl-4-(1-(2-methylbenzoyl)indolin-5-yl) thiazol-2-ylamino)-2-oxoethyl)phenoxy)ethyl)piperazine-1-carboxylate (0.20 g, 44% yield) as an off-white solid. MS (ESI) m/z: 696.3 [M+H]⁺.

### Step 4. N-(5-methyl-4-(1-(2-methylbenzoyl)indolin-5-yl)thiazol-2-yl)-2-(3-(2-(piperazin-1-yl)ethoxy)phenyl)acetamide

A solution of tert-butyl 4-(2-(3-(2-(5-methyl-4-(1-(2-methylbenzoyl)indolin-5-yl)thiazol-2-ylamino)-2-oxoethyl)phenoxy)ethyl)piperazine-1-carboxylate (0.20 g, 0.29 mmol) in hydrochloric acid (2 M in 1,4-dioxane, 20 mL) was stirred at room temperature for 16 hrs. The solvent was removed in vacuo. The residue was dissolved in dichloromethane (100 mL), washed with saturated sodium carbonate aqueous solution, dried over anhydrous sodium sulfate, filtered and concentrated to give N-(5-methyl-4-(1-(2-methylbenzoyl)indolin-5-yl)thiazol-2-yl)-2-(3-(2-(piperazin-1-yl)ethoxy)phenyl)acetamide (0.14 g, 82% yield) as an off-white solid. MS (ESI) m/z: 596.2 [M+H]⁺

### Step 5. 2-(3-(2-(4-(2-(2,6-dioxopiperidin-3-yl)-1,3-dioxoisoindolin-4-yl)piperazin-1-yl) ethoxy)phenyl)-N-(5-methyl-4-(1-(2-methylbenzoyl)indolin-5-yl)thiazol-2-yl)acetamide

A solution of N-(5-methyl-4-(1-(2-methylbenzoyl)indolin-5-yl)thiazol-2-yl)-2-(3-(2-(piperazin-1-yl) ethoxy)phenyl)acetamide (0.13 g, 0.22 mmol), 2-(2,6-dioxopiperidin-3-yl)-4-fluoroisoindoline-1,3-dione (0.18 g, 0.65 mmol) and N,N-diisopropylethylamine (0.11 g, 0.87 mmol) in 1-methylpyrrolidin-2-one (10 mL) was stirred at 120° C. for 18 hrs. The mixture was cooled to room temperature, diluted with water (15 mL), extracted with dichloromethane (30 mL×2), washed with brine (10 mL), dried over anhydrous sodium sulfate, filtered and concentrated in vacuo. The residue was purified by Prep-HPLC (Column: Welch Xtimate 21.2*250 mm C18, 10 μm, Mobile Phase: A: water (10 mmol/L ammonium bicarbonate) B: acetonitrile; B %: 30%-70% in 15.0 min) to give 2-(3-(2-(4-(2-(2,6-dioxopiperidin-3-yl)-1,3-dioxoisoindolin-4-yl)piperazin-1-yl)ethoxy)phenyl)-N-(5-methyl-4-(1-(2-methylbenzoyl)indolin-5-yl)thiazol-2-yl)acetamide (51.0 mg, 27%) as a yellow solid. ¹H NMR (400 MHz, DMSO-d₆): δ 12.34 & 12.25* (s, 1H), 11.09 (s, 1H), 8.23 & 5.52* (d, J=8.4 Hz, 1H), 7.69 (t, J=7.8 Hz, 1H), 7.55-7.50 (m, 1H), 7.52 (s, 1H), 7.36-7.30 (m, 6H), 7.24 (t, J=7.8 Hz, 1H), 6.96 (s, 1H) 6.91-6.86 (m, 2H), 5.09 (dd, J=12.8 Hz, 5.2 Hz, 1H), 4.21 & 3.71* (m, 2H), 4.12 (t, J=4.8 Hz, 2H), 3.71 (s, 2H), 3.32 (m, 3.14-3.11 (m, 2H), 2.88-2.83 (m, 1H), 2.78 (t, J=4.8 Hz, 2H), 2.69-2.67 (m, 2.60-2.56 (m, 2H), 2.45 & 2.33* (s, 3H), 2.29 & 2.18* (s, 3H), 2.03-1.98 (m, 1H). MS (ESI) m/z: 852.2[M+H]⁺ *Multiple signals arising from conformational isomers.

### Example 33

### Synthesis of 2-(3-(3-(4-(2-(2,6-dioxopiperidin-3-yl)-1,3-dioxoisoindolin-4-yl)piperazin-1-yl)propoxy)phenyl)-N-(5-methyl-4-(1-(2-methylbenzoyl)indolin-5-yl)thiazol-2-yl)acetamide (Cpd. No. 32)

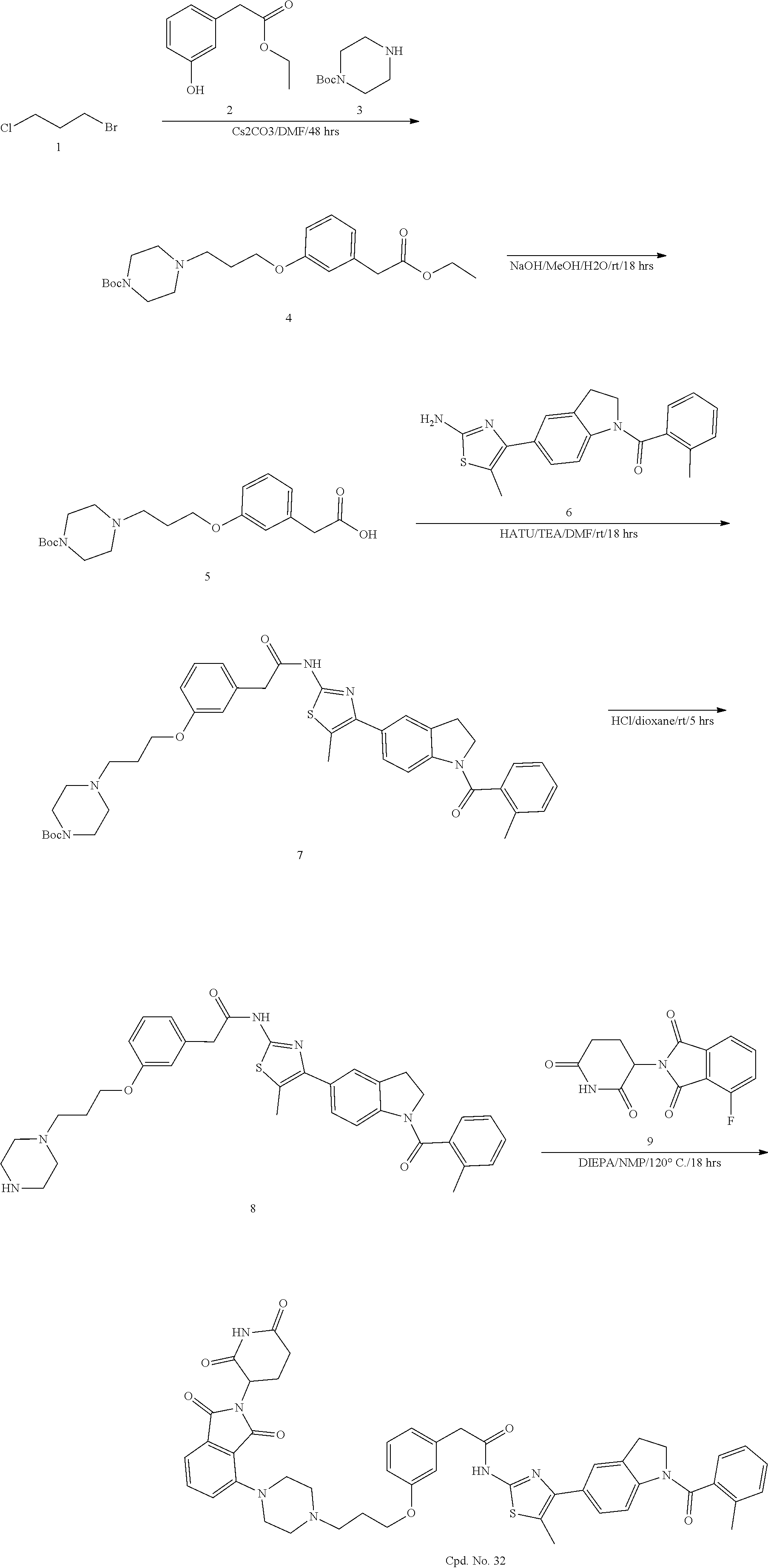

### Step 1. tert-butyl 4-(3-(3-(2-ethoxy-2-oxoethyl)phenoxy)propyl)piperazine-1-carboxylate

A solution of 1-bromo-3-chloropropane (250 g, 15.88 mmol), ethyl 2-(3-hydroxyphenyl)acetate (2.86 g, 15.9 mmol) and cesium carbonate (15.52 g, 47.6 mmol) in N, N-dimethylformamide (25 mL) was stirred at room temperature for 24 hrs. Tert-butyl piperazine-1-carboxylate (2.86 g, 15.4 mmol) was added into the resulting mixture and then the mixture was stirred at room temperature for 24 hrs. The mixture was diluted with water (60 mL), extracted with ethyl acetate (50 mL×2), washed with brine (20 mL×2), dried over anhydrous sodium sulfate, filtered and concentrated in vacuo. The residue was purified prep-HPLC (Column: Welch Xtimate 21.2*250 mm C18, 10 μm, Mobile Phase: A: water (10 mmol/L ammonium bicarbonate) B: acetonitrile; B %: 30%-70% in 15.0 min) to give tert-butyl 4-(3-(3-(2-ethoxy-2-oxoethyl)phenoxy)propyl)piperazine-1-carboxylate (0.85 g, 13% yield) as white solid. MS (ESI) m/z 407.3 [M+H]⁺.

### Step 2. 2-(3-(3-(4-(tert-butoxycarbonyl)piperazin-1-yl)propoxy)phenyl)acetic acid

A solution of tert-butyl 4-(3-(3-(2-ethoxy-2-oxoethyl)phenoxy)propyl)piperazine-1-carboxylate (0.85 g, 2.09 mmol), sodium hydroxide (0.25 g, 6.27 mmol) in methanol (15 mL) and water (15 mL) was stirred at room temperature for 18 hrs. The mixture was acidified with aqueous hydrochloric acid solution (2 M) to pH=3, extracted with dichloromethane (40 mL×3), dried over anhydrous sodium sulfate, filtered and concentrated in vacuo. The residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15.0 min) to give 2-(3-(3-(4-(tert-butoxycarbonyl)piperazin-1-yl)propoxy)phenyl)acetic acid (0.60 g, 76% yield) as a white solid. MS (ESI) m/z: 379.2 [M+H]⁺.

### Step 3. tert-butyl 4-(3-(3-(2-(5-methyl-4-(1-(2-methylbenzoyl)indolin-5-yl)thiazol-2-yl amino)-2-oxoethyl)phenoxy)propyl)piperazine-1-carboxylate

A solution of 2-(3-(3-(4-(tert-butoxycarbonyl)piperazin-1-yl)propoxy)phenyl)acetic acid (0.58 g, 1.53 mmol), (5-(2-amino-5-methylthiazol-4-yl)indolin-1-yl)(o-tolyl)methanone (0.80 g, 2.30 mmol), 2-(7-azabenzotriazol-1-yl)-N,N,N′,N′-tetramethyluronium hexafluorophosphate (0.87 g, 2.30 mmol) and triethylamine (0.62 g, 6.13 mmol) in N,N-dimethylformamide (10 mL) was stirred at room temperature for 18 hrs. The mixture was diluted with water (20 mL), extracted with dichloromethane (35 mL×2), washed with brine (25 mL), dried over anhydrous sodium sulfate, filtered and concentrated in vacuo. The residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15.0 min) to give tert-butyl 4-(3-(3-(2-(5-methyl-4-(1-(2-methylbenzoyl)indolin-5-yl)thiazol-2-ylamino)-2-oxoethyl) phenoxy)propyl)piperazine-1-carboxylate (0.32 g, 29% yield) as a white solid. MS (ESI) m/z: 710.2 [M+H]⁺.

### Step 4. N-(5-methyl-4-(1-(2-methylbenzoyl)indolin-5-yl)thiazol-2-yl)-2-(3-(3-(piperazin-1-yl)propoxy)phenyl)acetamide

A solution of tert-butyl 4-(3-(3-(2-(5-methyl-4-(1-(2-methylbenzoyl)indolin-5-yl)thiazol-2-ylamino)-2-oxoethyl)phenoxy)propyl)piperazine-1-carboxylate (0.32 g, 0.45 mmol) in hydrochloric acid (2 M in 1,4-dioxane, 25 mL) was stirred at room temperature for 5 hrs. The solvent was removed in vacuo. The residue was dissolved in dichloromethane (70 mL), washed with saturated sodium carbonate aqueous solution, dried over anhydrous sodium sulfate, filtered and concentrated to give N-(5-methyl-4-(1-(2-methylbenzoyl)indolin-5-yl)thiazol-2-yl)-2-(3-(3-(piperazin-1-yl)propoxy)phenyl)acetamide (0.27 g, 98% yield) as an off-white solid. MS (ESI) m/z: 610.2 [M+H]⁺

### Step 5. 2-(3-(3-(4-(2-(2,6-dioxopiperidin-3-yl)-1,3-dioxoisoindolin-4-yl)piperazin-1-yl) propoxy)phenyl)-N-(5-methyl-4-(1-(2-methylbenzoyl)indolin-5-yl)thiazol-2-yl)acetamide

A solution of N-(5-methyl-4-(1-(2-methylbenzoyl)indolin-5-yl)thiazol-2-yl)-2-(3-(3-(piperazin-1-yl) propoxy)phenyl)acetamide (0.26 g, 0.43 mmol), 2-(2,6-dioxopiperidin-3-yl)-4-fluoroisoindoline-1,3-dione (0.35 g, 1.28 mmol) and N,N-diisopropylethylamine (0.22 g, 1.71 mmol) in 1-methylpyrrolidin-2-one (10 mL) was stirred at 120° C. for 18 hrs. The mixture was cooled to room temperature, diluted with water (20 mL), extracted with dichloromethane (30 mL×2), washed with brine (20 mL), dried over anhydrous sodium sulfate, filtered and concentrated in vacuo. The residue was purified by Prep-HPLC (Column: Welch Xtimate 21.2*250 mm C18, 10 μm, Mobile Phase: A: water (10 mmol/L ammonium bicarbonate) B: acetonitrile; B %: 30%-70% in 15.0 min) to give 2-(3-(3-(4-(2-(2,6-dioxopiperidin-3-yl)-1,3-dioxoisoindolin-4-yl)piperazin-1-yl)propoxy)phenyl)-N-(5-methyl-4-(1-(2-methylbenzoyl)indolin-5-yl)thiazol-2-yl)acetamide (0.18 g, 48.8%) as a yellow solid. ¹H NMR (400 MHz, DMSO-d₆): δ 12.34 & 12.25* (s, 1H), 11.09 (s, 1H), 8.23 & 5.52* (d, J=8.8 Hz, 1H), 7.69 (t, J=7.8 Hz, 1H), 7.52 & 7.04* (m, 1H), 7.52 (s, 1H), 7.36-7.30 (m, 6H), 7.24 (t, J=7.8 Hz, 1H), 6.93-6.83 (m, 3H), 5.09 (dd, J=12.6 Hz, 5.4 Hz, 1H), 4.21 & 3.73* (m, 2H), 4.03 (t, J=6.0 Hz, 2H), 3.71 (s, 2H), 3.30 (brs, 4H), 3.12 (t, J=8.4 Hz, 2H), 2.88-2.82 (m, 1H), 2.57 (m, 8H), 2.45 & 2.33* (s, 3H), 2.29 & 2.18* (s, 3H), 2.03-1.98 (m, 1H), 1.95-1.87 (m, 2H). MS (ESI) m/z: 865.8 [M+H]⁺. *Multiple signals arising from conformational isomers.

### Example 34

### Synthesis of 2-(3-(4-(4-(2-(2,6-dioxopiperidin-3-yl)-1,3-dioxoisoindolin-4-yl)piperazin-1-yl)butoxy)phenyl)-N-(5-methyl-4-(1-(2-methylbenzoyl)indolin-5-yl)thiazol-2-yl)acetamide (Cpd. No. 33)

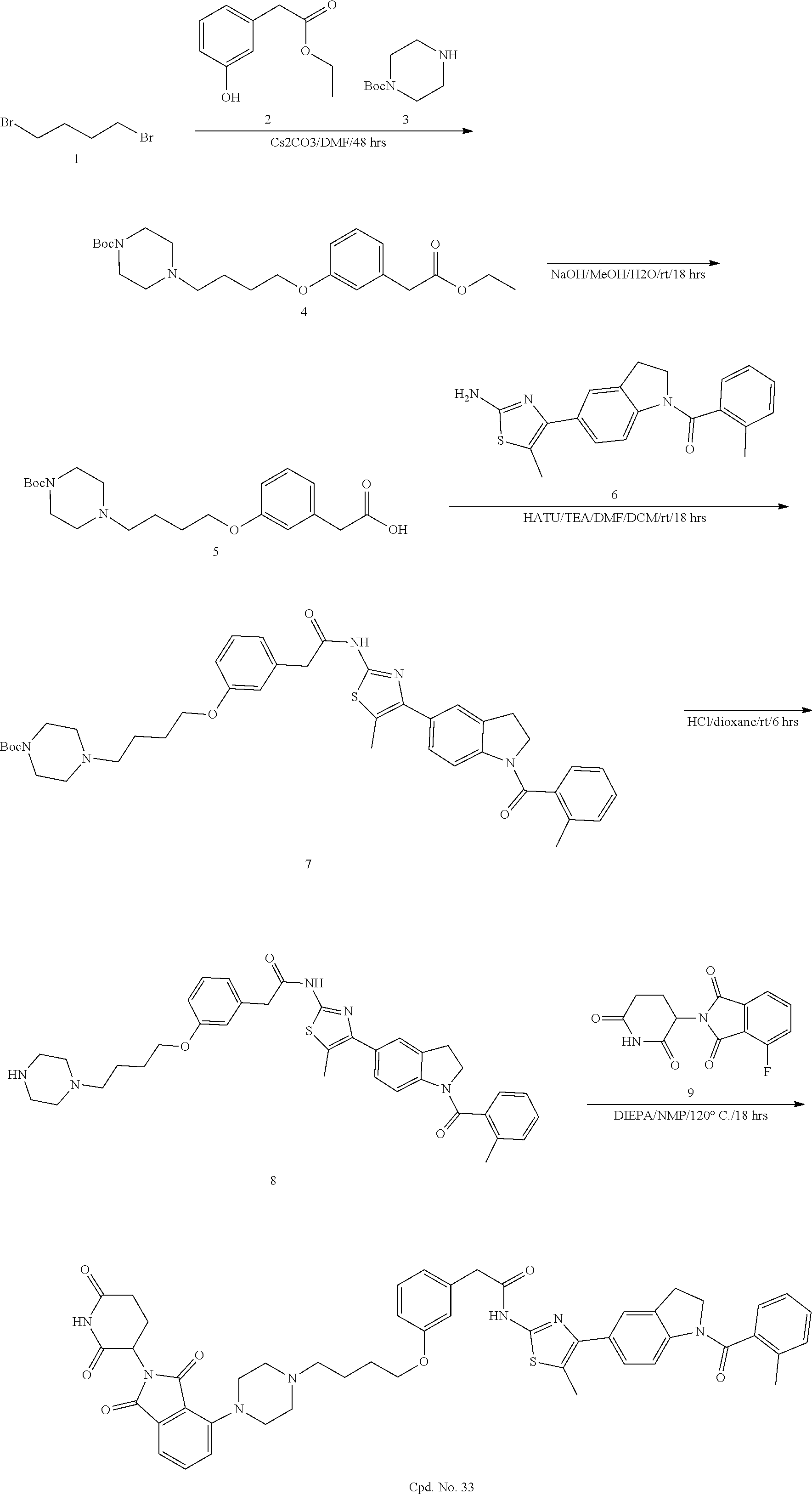

### Step 1. tert-butyl 4-(4-(3-(2-ethoxy-2-oxoethyl)phenoxy)butyl)piperazine-1-carboxylate

A solution of 1,4-dibromobutane (1.50 g, 6.95 mmol), ethyl 2-(3-hydroxyphenyl)acetate (1.25 g, 6.94 mmol) and cesium carbonate (6.79 g, 20.84 mmol) in N, N-dimethylformamide (20 mL) was stirred at room temperature for 24 hrs. Tert-butyl piperazine-1-carboxylate (1.29 g, 6.95 mmol) was added into the resulting mixture and then the mixture was stirred at room temperature for 24 hrs. The mixture was diluted with water (50 mL), extracted with ethyl acetate (50 mL×2), washed with brine (20 mL×2), dried over anhydrous sodium sulfate, filtered and concentrated in vacuo. The residue was purified prep-HPLC (Column: Welch Xtimate 21.2*250 mm C18, 10 μm, Mobile Phase: A: water (10 mmol/L ammonium bicarbonate) B: acetonitrile; B %: 30%-70% in 15.0 min) to give tert-butyl 4-(4-(3-(2-ethoxy-2-oxoethyl)phenoxy)butyl)piperazine-1-carboxylate (0.37 g, 13% yield) as off-white solid. MS (ESI) m/z 421.3 [M+H]⁺.

### Step 2. 2-(3-(4-(4-(tert-butoxycarbonyl)piperazin-1-yl)butoxy)phenyl)acetic acid

A solution of tert-butyl 4-(4-(3-(2-ethoxy-2-oxoethyl)phenoxy)butyl)piperazine-1-carboxylate (0.37 g, 0.88 mmol), sodium hydroxide (0.11 g, 2.64 mmol) in methanol (10 mL) and water (10 mL) was stirred at room temperature for 18 hrs. The mixture was acidified with aqueous hydrochloric acid solution (2 M) to pH=3, extracted with dichloromethane (30 mL×4), dried over anhydrous sodium sulfate, filtered and concentrated in vacuo. The residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15.0 min) to give 2-(3-(4-(4-(tert-butoxycarbonyl)piperazin-1-yl)butoxy)phenyl) acetic acid (0.10 g, 29% yield) as a white solid. MS (ESI) m/z: 393.1 [M+H]⁺.

### Step 3. tert-butyl 4-(4-(3-(2-((5-methyl-4-(1-(2-methylbenzoyl)indolin-5-yl)thiazol-2-yl) amino)-2-oxoethyl)phenoxy)butyl)piperazine-1-carboxylate

A solution of 2-(3-(4-(4-(tert-butoxycarbonyl)piperazin-1-yl)butoxy)phenyl)acetic acid (0.10 g, 0.25 mmol), (5-(2-amino-5-methylthiazol-4-yl)indolin-1-yl)(o-tolyl)methanone (0.14 g, 0.41 mmol), 2-(7-azabenzotriazol-1-yl)-N,N,N′N-tetramethyluronium hexafluorophosphate (0.16 g, 0.41 mmol) and triethylamine (0.10 g, 1.02 mmol) in N,N-dimethylformamide (4 mL) was stirred at room temperature for 18 hrs. The mixture was diluted with water (10 mL), extracted with dichloromethane (20 mL×2), washed with brine (10 mL), dried over anhydrous sodium sulfate, filtered and concentrated in vacuo. The residue was purified by prep-HPLC (Column: Welch Xtimate 21.2*250 mm C18, 10 jam, Mobile Phase: A: water (10 mmol/L ammonium bicarbonate) B: acetonitrile; B %: 30%-70% in 15.0 min) to give tert-butyl 4-(4-(3-(2-((5-methyl-4-(1-(2-methylbenzoyl)indolin-5-yl)thiazol-2-yl)amino)-2-oxoethyl)phenoxy)butyl)piperazine-1-carboxylate (25.0 mg, 14% yield) as an off-white solid. MS (ESI) m/z: 724.3 [M+H]⁺.

### Step 4. N-(5-methyl-4-(1-(2-methylbenzoyl)indolin-5-yl)thiazol-2-yl)-2-(3-(4-(piperazin-1-yl)butoxy)phenyl)acetamide

A solution of tert-butyl 4-(4-(3-(2-((5-methyl-4-(1-(2-methylbenzoyl)indolin-5-yl)thiazol-2-yl) amino)-2-oxoethyl)phenoxy)butyl)piperazine-1-carboxylate (25.0 mg, 0.035 mmol) in hydrochloric acid (2 M in 1,4-dioxane, 5 mL) was stirred at room temperature for 6 hrs. The solvent was removed in vacuo. The residue was dissolved in dichloromethane (40 mL), washed with saturated sodium carbonate aqueous solution, dried over anhydrous sodium sulfate, filtered and concentrated to give N-(5-methyl-4-(1-(2-methylbenzoyl)indolin-5-yl)thiazol-2-yl)-2-(3-(4-(piperazin-1-yl)butoxy)phenyl)acetamide (17.0 mg, 79% yield) as an off-white solid. MS (ESI) m/z: 624.3 [M+H]⁺

### Step 5. 2-(3-(4-(4-(2-(2,6-dioxopiperidin-3-yl)-1,3-dioxoisoindolin-4-yl)piperazin-1-yl) butoxy)phenyl)-N-(5-methyl-4-(1-(2-methylbenzoyl)indolin-5-yl)thiazol-2-yl)acetamide

A solution of A-(5-methyl-4-(1-(2-methylbenzoyl)indolin-5-yl)thiazol-2-yl)-2-(3-(4-(piperazin-1-yl)butoxy)phenyl)acetamide (17.0 mg, 0.027 mmol), 2-(2,6-dioxopiperidin-3-yl)-4-fluoroisoindoline-1,3-dione (23.0 mg, 0.08 mmol) and N,N-diisopropylethylamine (14.0 mg, 0.11 mmol) in 1-methylpyrrolidin-2-one (2 mL) was stirred at 120° C. for 18 hrs. The mixture was cooled to room temperature, diluted with water (10 mL), extracted with dichloromethane (20 mL×2), washed with brine (10 mL), dried over anhydrous sodium sulfate, filtered and concentrated in vacuo. The residue was purified by Prep-HPLC (Column: Welch Xtimate 21.2*250 mm C18, 10 μm, Mobile Phase: A: water (10 mmol/L ammonium bicarbonate) B: acetonitrile; B %: 30%-70% in 15.0 min) to give 2-(3-(4-(4-(2-(2,6-dioxopiperidin-3-yl)-1,3-dioxoisoindolin-4-yl)piperazin-1-yl)butoxy)phenyl)-N-(5-methyl-4-(1-(2-methylbenzoyl)indolin-5-yl)thiazol-2-yl)acetamide (5.0 mg, 21%) as a yellow solid. ¹H NMR (400 MHz, DMSO-d₆): δ 12.34 (brs, 1H), 11.09 (s, 1H), 8.23 & 5.52* (d, J=8.0 Hz, 1H), 7.68 (t, J=7.8 Hz, 1H), 7.52 (s, 2H), 7.37-7.32 (m, 6H), 7.23 (t, J=7.8 Hz, 1H), 6.89-6.83 (m, 3H), 5.09 (dd, J=12.6 Hz, 5.4 Hz, 1H), 4.21 & 3.73* (m, 2H), 4.00 (t, J=6.0 Hz, 2H), 3.70 (s, 2H), 3.28 (m, 3.13 (t, J=7.6 Hz, 2H), 2.87-2.84 (m, 1H), 2.60 (m, 6H), 2.44 & 2.33* (s, 3H), 2.40 (t, J=6.8 Hz, 2H), 2.29 & 2.18* (s, 3H), 2.03-1.98 (m, 1 H), 1.78-1.70 (m, 2H), 1.68-1.57 (m, 2H). MS (ESI) m/z: 880.3 [M+H]⁺. *Multiple signals arising from conformational isomers.

### Example 35

### Synthesis of N-(4-(1-(cyclopropanecarbonyl)indolin-5-yl)-5-methylthiazol-2-yl)-2-(3-(2-(2-(2-(2,6-dioxopiperidin-3-yl)-1,3-dioxoisoindolin-4-ylamino)ethoxy)ethoxy)phenyl)acetamide (Cpd. No. 34)

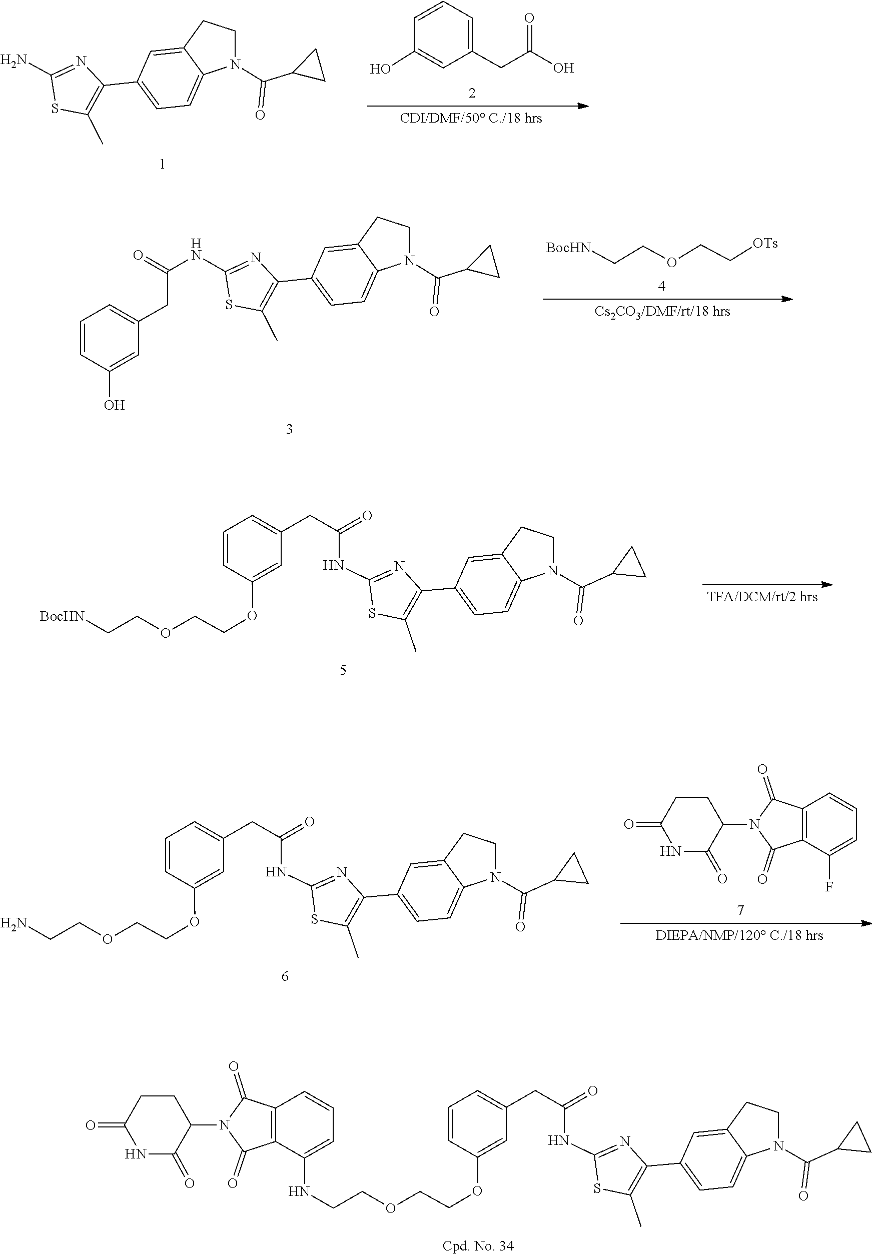

### Step 1. N-(4-(1-(cyclopropanecarbonyl)indolin-5-yl)-5-methylthiazol-2-yl)-2-(3-hydroxyphenyl)acetamide

A solution of 2-(3-hydroxyphenyl)acetic acid (0.51 g, 3.4 mmol), di(1H-imidazol-1-yl)methanone (0.82 g, 5.0 mmol) and N, N-diisopropylethylamine (1.73 g, 13.4 mmol) in N,N-dimethylformamide (20 mL) was stirred at room temperature for 30 min. (5-(2-amino-5-methylthiazol-4-yl)indolin-1-yl)(cyclopropyl)methanone (1.00 g, 3.34 mmol) was added into the resulting mixture. The mixture was stirred at 50° C. for 18 hrs. The mixture was cooled to room temperature, diluted with water (50 mL), extracted with dichloromethane (50 mL×2), dried over anhydrous sodium sulfate, filtered and concentrated in vacuo. The residue was purified by flash column chromatography (dichloromethane/methanol=5/95) to give N-(4-(1-(cyclopropanecarbonyl)indolin-5-yl)-5-methylthiazol-2-yl)-2-(3-hydroxyphenyl)acetamide (0.62 g, 43% yield) as a yellow solid. MS (ESI) m/z: 434.1 [M+H]⁺

### Step 2. tert-butyl 2-(2-(3-(2-(4-(1-(cyclopropanecarbonyl)indolin-5-yl)-5-methylthiazol-2-ylamino)-2-oxoethyl)phenoxy)ethoxy)ethylcarbamate

A solution of N-(4-(1-(cyclopropanecarbonyl)indolin-5-yl)-5-methylthiazol-2-yl)-2-(3-hydroxyphenyl)acetamide (0.57 g, 1.3 mmol), 2-(2-((tert-butoxycarbonyl)amino)ethoxy)ethyl 4-methylbenzenesulfonate (0.57 g, 1.6 mmol), cesium carbonate (0.86 g, 2.63 mmol) in N,N-dimethylformamide (20 mL) was stirred at room temperature for 18 brs. The mixture was quenched with water (50 mL), extracted with dichloromethane (50 mL×2), dried over anhydrous sodium sulfate, filtered and concentrated in vacuo. The residue was purified by flash column chromatography (dichloromethane/methane=3/97) to give tert-butyl 2-(2-(3-(2-(4-(1-(cyclopropanecarbonyl)indolin-5-yl)-5-methylthiazol-2-ylamino)-2-oxoethyl)phenoxy)ethoxy)ethylcarbamate (0.48 g, 59% yield) as a white solid. MS (ESI) m/z: 621.0 [M+H]⁺

### Step 3. 2-(3-(2-(2-aminoethoxy)ethoxy)phenyl)-AN-(4-(1-(cyclopropanecarbonyl)indolin-5-yl)-5-methylthiazol-2-yl)acetamide

A solution of tert-butyl 2-(2-(3-(2-(4-(1-(cyclopropanecarbonyl)indolin-5-yl)-5-methylthiazol-2-ylamino)-2-oxoethyl)phenoxy)ethoxy)ethylcarbamate (0.48 g, 0.77 mmol) in trifluoracetic acid (10 mL) and dichloromethane (30 mL) was stirred at room temperature for 2 hrs. The solvent was removed in vacuo, the residue was dissolved in dichloromethane (100 mL), washed with saturated sodium carbonate aqueous solution, dried over anhydrous sodium sulfate, filtered and concentrated in vacuo. The residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in min) to give 2-(3-(2-(2-aminoethoxy)ethoxy)phenyl)-N-(4-(1-(cyclopropanecarbonyl)indolin-5-yl)-5-methylthiazol-2-yl)acetamide (0.12 g, 30% yield) as a yellow solid. MS (ESI) m/z 521.0 [M+H]⁺

### Step 4. N-(4-(1-(cyclopropanecarbonyl)indolin-5-yl)-5-methylthiazol-2-yl)-2-(3-(2-(2-(2-(2,6-dioxopiperidin-3-yl)-1,3-dioxoisoindolin-4-ylamino)ethoxy)ethoxy)phenyl)acetamide

A solution of 2-(3-(2-(2-aminoethoxy)ethoxy)phenyl)-N-(4-(1-(cyclopropanecarbonyl)indolin-5-yl)-5-methylthiazol-2-yl)acetamide (0.10 g, 0.19 mmol), 2-(2,6-dioxopiperidin-3-yl)-4-fluoroisoindoline-1,3-dione (0.27 g, 0.96 mmol) and N, N-diisopropylethylamine (0.20 g, 1.54 mmol) in 1-methylpyrrolidin-2-one (5 mL) was stirred at 120° C. for 18 hrs. The mixture was cooled to room temperature, diluted with water (20 mL), extracted with dichloromethane (20 mL×3), washed with brine (15 mL), dried over anhydrous sodium sulfate, filtered and concentrated in vacuo. The residue was purified by Prep-HPLC (Column: Welch Xtimate 21.2*250 mm C18, 10 μm, Mobile Phase: A: water (10 mmol/L ammonium bicarbonate) B: acetonitrile; B %: 30%-70% in 15 min) to give N-(4-(1-(cyclopropanecarbonyl)indolin-5-yl)-5-methylthiazol-2-yl)-2-(3-(2-(2-(2-(2,6-dioxopiperidin-3-yl)-1,3-dioxoisoindolin-4-ylamino)ethoxy)ethoxy)phenyl)acetamide (21.3 mg, 14% yield) as a yellow solid. ¹H NMR (400 MHz, DMSO-d₆): δ 12.31 (s, 1H), 11.11 (s, 1H), 8.07 (d, J=8.0 Hz, 1H), 7.55 (dd, J=9.2 Hz, 7.2 Hz, 1H), 7.49 (brs, 1H), 7.41 (d, J=8.0 Hz, 1H), 7.22 (t, J=8.0 Hz, 1H), 7.14 (d, J=8.8 Hz, 1H), 7.02 (d, J=7.2 Hz, 1H), 6.91 (brs, 1H), 6.88 (d, J=7.6 Hz, 1H), 6.83 (dd, J=8.4, 2.0 Hz, 1H), 6.65 (t, J=5.6 Hz, 1H), 5.05 (dd, J=12.8 Hz, 5.6 Hz, 1H), 4.33 (t, J=8.0 Hz, 1H), 4.09 (t, J=4.0 Hz, 2H), 3.78 (t, J=4.4 Hz, 2H), 3.68-3.67 (m, 3.49 (q, J=5.6 Hz, 2H), 3.22 (t, J=8.4 Hz, 2H), 2.89-2.84 (m, 1H), 2.59-2.51 (m, 2H), 2.43 (s, 3H), 2.08-1.93 (m, 2H), 0.87-0.85 (m, 4H). MS (ESI) m/z: 777.0 [M+H]⁺.

### Example 36

### Synthesis of 2-(3-(2-(2-(2-(2,6-dioxopiperidin-3-yl)-1,3-dioxoisoindolin-4-ylamino)ethoxy)ethoxy)phenyl)-N-(5-methyl-4-(1-(1-methyl-1H-imidazole-5-carbonyl)indolin-5-yl)thiazol-2-yl)acetamide (Cpd. No. 35)

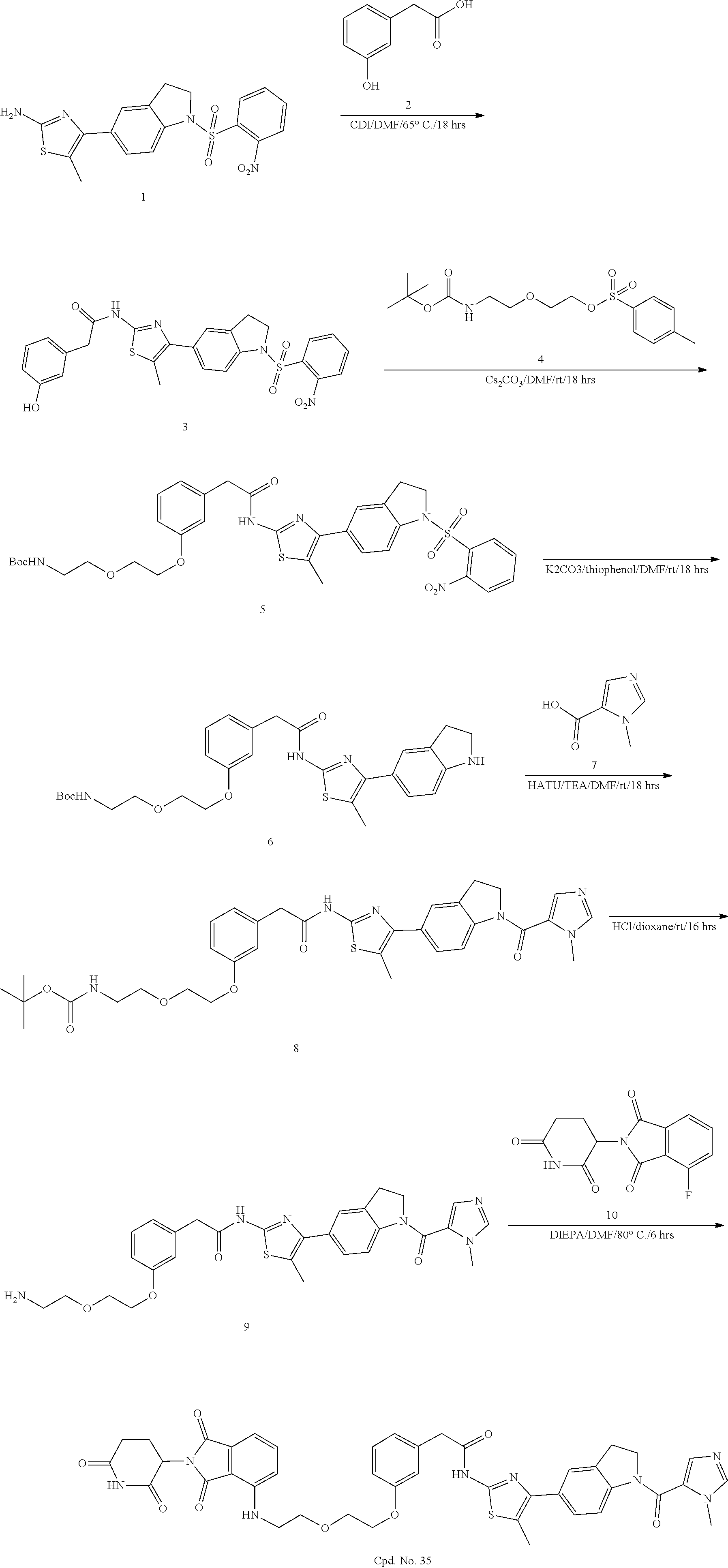

### Step 1. 2-(3-hydroxyphenyl)-N-(5-methyl-4-(1-(2-nitrophenylsulfonyl)indolin-5-yl)thiazol-2-yl)acetamide

A solution of 2-(3-hydroxyphenyl)acetic acid (1.83 g, 12.0 mmol), di(1H-imidazol-1-yl)methanone (2.92 g, 18.0 mmol) and N, N-diisopropylethylamine (4.65 g, 36.0 mmol) in N,N-dimethylformamide (50 mL) was stirred at room temperature for 30 mins. 5-Methyl-4-(1-((2-nitrophenyl)sulfonyl)indolin-5-yl) thiazol-2-amine (5.00 g, 12.0 mmol) was added into the resulting mixture. The mixture was stirred at 65° C. for 18 hrs. The mixture was cooled to room temperature, diluted with water (100 mL), extracted with dichloromethane (50 mL×2), dried over anhydrous sodium sulfate, filtered and concentrated in vacuo. The residue was purified by flash column chromatography (dichloromethane/methanol=4/96) to give 2-(3-hydroxyphenyl)-N-(5-methyl-4-(1-((2-nitrophenyl)sulfonyl)indolin-5-yl)thiazol-2-yl)acetamide (2.03 g, 30.7% yield) as a yellow solid. MS (ESI) m/z: 551.1 [M+H]⁺.

### Step 2. tert-butyl 2-(2-(3-(2-(5-methyl-4-(1-(2-nitrophenylsulfonyl)indolin-5-yl)thiazol-2-ylamino)-2-oxoethyl)phenoxy)ethoxy)ethylcarbamate

A solution of 2-(3-hydroxyphenyl)-N-(5-methyl-4-(1-((2-nitrophenyl)sulfonyl)indolin-5-yl)thiazol-2-yl)acetamide (1.87 g, 3.40 mmol), 2-(2-((tert-butoxycarbonyl)amino)ethoxy)ethyl 4-methylbenzenesulfonate (1.22 g, 3.40 mmol), cesium carbonate (2.21 g, 6.80 mmol) in N,N-dimethylformamide (20 mL) was stirred at room temperature for 18 hrs. The mixture was quenched with water (50 mL), extracted with dichloromethane (50 mL×2), dried over anhydrous sodium sulfate, filtered and concentrated in vacuo. The residue was purified by flash column chromatography (dichloromethane/methanol=3/97) to give tert-butyl 2-(2-(3-(2-(5-methyl-4-(1-(2-nitrophenylsulfonyl)indolin-5-yl)thiazol-2-ylamino)-2-oxoethyl)phenoxy)ethoxy)ethylcarbamate (0.89 g, 35% yield) as a yellow solid. MS (ESI) m/z 738.0 [M+H]⁺.

### Step 3. tert-butyl 2-(2-(3-(2-(4-(indolin-5-yl)-5-methylthiazol-2-ylamino)-2-oxoethyl)phenoxy)ethoxy)ethylcarbamate

A solution of tert-butyl 2-(2-(3-(2-(5-methyl-4-(1-(2-nitrophenylsulfonyl)indolin-5-yl)thiazol-2-ylamino)-2-oxoethyl)phenoxy)ethoxy)ethylcarbamate (0.80 g, 1.1 mmol), potassium carbonate (0.30 g, 2.2 mmol), thiophenol (0.14 g, 1.3 mmol) in N,N-dimethylformamide (20 mL) was stirred at room temperature for 18 hrs. The mixture was diluted with water (40 mL), extracted with dichloromethane (50 mL×2), dried over anhydrous sodium sulfate, filtered and concentrated in vacuo. The residue was purified by flash column chromatography (dichloromethane/methanol=20/1) to give tert-butyl 2-(2-(3-(2-(4-(indolin-5-yl)-5-methylthiazol-2-ylamino)-2-oxoethyl)phenoxy)ethoxy)ethylcarbamate (0.41 g, 68% yield) as a yellow solid. MS (ESI) m/z: 552.9 [M+H]⁺.

### Step 4. tert-butyl 2-(2-(3-(2-(5-methyl-4-(1-(1-methyl-11-imidazole-5-carbonyl)indolin-5-yl)thiazol-2-ylamino)-2-oxoethyl)phenoxy)ethoxy)ethylcarbamate

A solution of 1-methyl-1H-imidazole-5-carboxylic acid (93.2 mg, 0.74 mmol), tert-butyl 2-(2-(3-(2-(4-(indolin-5-yl)-5-methylthiazol-2-ylamino)-2-oxoethyl)phenoxy)ethoxy)ethylcarbamate (0.41 g, 0.74 mmol), 2-(7-azabenzotriazol-1-yl)-N,N,N′,N′-tetramethyluronium hexafluorophosphate (0.56 g, 1.5 mmol) and triethylamine (0.22 g, 2.2 mmol) in N,N-dimethylformamide (10 mL) was stirred at room temperature for 18 hrs. The mixture was diluted with water (20 mL), extracted with dichloromethane (30 mL×3), washed with brine (30 mL), dried over anhydrous sodium sulfate, filtered and concentrated in vacuo. The residue was purified by flash column chromatography (dichloromethane/methanol=8/92) to give tert-butyl 2-(2-(3-(2-(5-methyl-4-(1-(1-methyl-1H-imidazole-5-carbonyl)indolin-5-yl)thiazol-2-ylamino)-2-oxoethyl)phenoxy)ethoxy)ethylcarbamate (0.20 g, 41% yield) as white solid. MS (ESI) m/z: 660.9 [M+H]⁺

### Step 5. 2-(3-(2-(2-aminoethoxy)ethoxy)phenyl)-N-(5-methyl-4-(1-(1-methyl-1H-imidazole-5-carbonyl)indolin-5-yl)thiazol-2-yl)acetamide

A solution of tert-butyl 2-(2-(3-(2-(5-methyl-4-(1-(1-methyl-1H-imidazole-5-carbonyl)indolin-5-yl)thiazol-2-ylamino)-2-oxoethyl)phenoxy)ethoxy)ethylcarbamate (0.20 g, 0.30 mmol) and hydrochloric acid (2 M in 1,4-dioxane, 2 mL) in 1,4-dioxane (2 mL) was stirred at room temperature for 16 hrs. The solvent was removed in vacuo and the residue was dissolved in dichloromethane (50 mL), washed with saturated sodium carbonate aqueous solution, dried over anhydrous sodium sulfate, filtered and concentrated in vacuo. The residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15 min) to give 2-(3-(2-(2-aminoethoxy)ethoxy)phenyl)-N-(5-methyl-4-(1-(1-methyl-1H-imidazole-5-carbonyl)indolin-5-yl)thiazol-2-yl)acetamide (0.12 g, 71% yield) as a white solid. MS (ESI) m/z 560.9 [M+H]⁺.

### Step 6. 2-(3-(2-(2-(2-(2,6-dioxopiperidin-3-yl)-1,3-dioxoisoindolin-4-ylamino)ethoxy)ethoxy)phenyl)-N-(5-methyl-4-(1-(1-methyl-1H-imidazole-5-carbonyl)indolin-5-yl)thiazol-2-yl)acetamide

A solution of 2-(3-(2-(2-aminoethoxy)ethoxy)phenyl)-N-(5-methyl-4-(1-(1-methyl-1H-imidazole-5-carbonyl)indolin-5-yl)thiazol-2-yl)acetamide (0.12 g, 0.21 mmol), 2-(2,6-dioxopiperidin-3-yl)-4-fluoroisoindoline-1,3-dione (0.30 g, 1.1 mmol) and N, N-diisopropylethylamine (0.14 g, 1.1 mmol) in 1-methylpyrrolidin-2-one (5 mL) was stirred at 80° C. for 6 hrs. The mixture was cooled to room temperature, diluted with water (10 mL), extracted with dichloromethane (20 mL×2), washed with brine (10 mL), dried over anhydrous sodium sulfate, filtered and concentrated in vacuo. The residue was purified by Prep-HPLC (Column: Welch Xtimate 21.2*250 mm C18, 10 μm. Mobile Phase: A: water (10 mmol/L ammonium bicarbonate) B: acetonitrile; B %: 30%-70% in min) to give 2-(3-(2-(2-((2-(2,6-dioxopiperidin-3-yl)-1,3-dioxoisoindolin-4-yl)amino)ethoxy)ethoxy)phenyl)-N-(5-methyl-4-(1-(1-methyl-1H-imidazole-5-carbonyl)indolin-5-yl)thiazol-2-yl)acetamide (25.5 mg, 15% yield) as a yellow solid. ¹H NMR (400 MHz, DMSO-d₆): δ 11.19 (brs, 2H), 8.07 (d, J=8.4 Hz, 1H), 7.84 (s, 1H), 7.59 (s, 1H), 7.57-7.53 (m, 2H), 7.49 (d, J=8.4 Hz, 1H), 7.22 (t, J=8.0 Hz, 1H), 7.14 (d, J=8.4 Hz, 1H), 7.02 (d, J=6.8 Hz, 1H), 6.92-6.88 (m, 2H), 6.83 (dd, J=8.4, 2.0 Hz, 1H), 6.64 (t, J=6.0 Hz, 1H), 5.05 (dd, J=12.8, 5.2 Hz, 1H), 4.36 (t, J=8.0 Hz, 2H), 4.09 (t, J=4.4 Hz, 2H), 3.79 (s, 5H), 3.69 (s, 4H), 3.50 (q, J=5.2 Hz, 2H), 3.19 (t, J=8.4 Hz, 2H), 2.92-2.82 (m, 1H), 2.67-2.52 (m, 2H), 2.45 (s, 3H), 2.04-1.97 (m, 1H). MS (ESI) m/z: 817.3 [M+H]⁺.

### Example 37

### Synthesis of N-(4-(1-(cyclopropanecarbonyl)indolin-5-yl)-5-methylthiazol-2-yl)-2-(3-(2-(2-(2-(2,6-dioxopiperidin-3-yl)-1,3-dioxoisoindolin-4-ylamino)ethoxy)ethoxy)-4-fluorophenyl)acetamide (Cpd. No. 36)

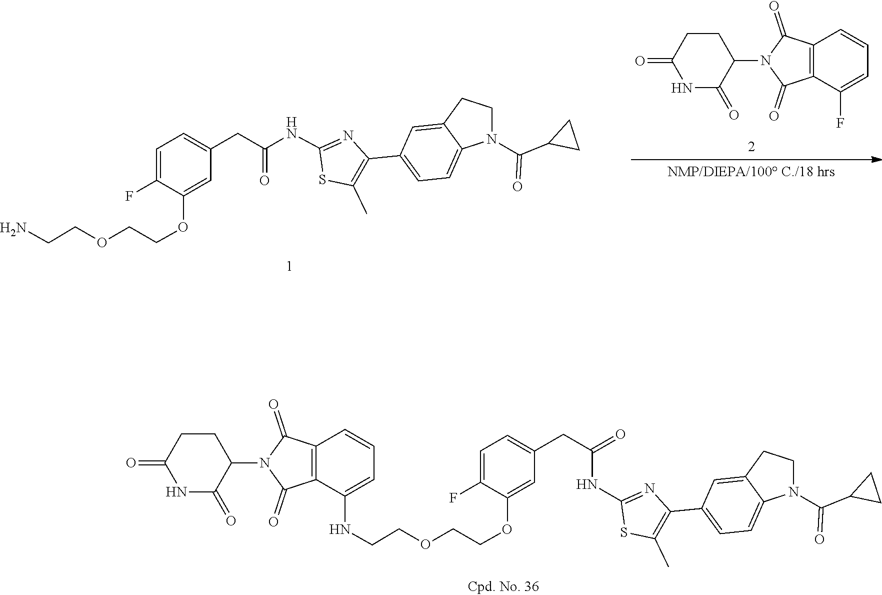 [SMILES: Cc1sc(NC(=O)Cc2ccc(F)c(OCCOCCN)c2)nc1-c1ccc2c(c1)CCN2C(=O)C1CC1 ; O=C1CCC(N2C(=O)c3cccc(F)c3C2=O)C(=O)N1 ; Cc1sc(NC(=O)Cc2ccc(F)c(OCCOCCNc3cccc4c3C(=O)N(C3CCC(=O)NC3=O)C4=O)c2)nc1-c1ccc2c(c1)CCN2C(=O)C1CC1]

### Step 1. N-(4-(1-(cyclopropanecarbonyl)indolin-5-yl)-5-methylthiazol-2-yl)-2-(3-(2-(2-(2-(2,6-dioxopiperidin-3-yl)-1,3-dioxoisoindolin-4-ylamino)ethoxy)ethoxy)-4-fluorophenyl)acetamide

A solution of 2-(3-(2-(2-aminoethoxy)ethoxy)-4-fluorophenyl)-N-(4-(1-(cyclopropanecarbonyl)indolin-5-yl)-5-methylthiazol-2-yl)acetamide (0.20 g, 0.37 mmol), 2-(2,6-dioxopiperidin-3-yl)-4-fluoroisoindoline-1,3-dione (0.51 g, 1.9 mmol) and N,N-diisopropylethylamine (0.29 g, 2.2 mmol) in 1-methylpyrrolidin-2-one (10 mL) was stirred at 100° C. for 18 hrs. The mixture was diluted with water (20 mL), extracted with ethyl acetate (25 mL×4), dried over anhydrous sodium sulfate, filtered and concentrated in vacuo. The residue was purified by Prep-HPLC (Column: Welch Xtimate 21.2*250 mm C18, 10 μm, Mobile Phase: A: water (10 mmol/L ammonium bicarbonate) B: acetonitrile; B %: 30%-70% in 15.0 min) to give N-(4-(1-(cyclopropanecarbonyl)indolin-5-yl)-5-methylthiazol-2-yl)-2-(3-(2-(2-(2-(2,6-dioxopiperidin-3-yl)-1,3-dioxoisoindolin-4-ylamino)ethoxy)ethoxy)-4-fluorophenyl)acetamide (64.3 mg, 22% yield) as a yellow solid. ¹H NMR (400 MHz. DMSO-d₆): δ 12.28 (s, 1H), 11.09 (s, 1H), 8.06 (d, J=8.0 Hz, 1H), 7.54 (dd, J=8.4 Hz, 7.6 Hz, 2H), 7.49 (s, 1H), 7.41 (d, J=8.0 Hz, 1H), 7.14 (dd, J=10.0 Hz, 8.4 Hz, 3H), 7.01 (d, J=6.8 Hz, 1H), 6.89-6.86 (m, 1H), 6.63 (t, J=6.0 Hz, 1H), 5.04 (dd, J=12.8 Hz, 5.6 Hz, 1H), 4.31 (t, J=6.8 Hz, 2H), 4.17 (t, J=3.6 Hz, 2H), 3.81 (t, J=4.0 Hz, 2H), 3.70-3.68 (m, 3.48 (q, J=5.2 Hz, 2H), 3.22 (t, J=8.0 Hz, 2H), 2.87-2.83 (m, 1H), 2.67-2.54 (m, 2H), 2.42 (s, 3H), 2.01-1.96 (m, 2H), 0.88-0.86 (m, 4H). MS (ESI) m/z: 759.1 [M+H]⁺

### Example 38

### Synthesis of 2-(3-(2-(2-((2-(2,6-dioxopiperidin-3-yl)-1,3-dioxoisoindolin-4-yl)amino)ethoxy)ethoxy)-4-fluorophenyl)-N-(5-methyl-4-(1-(1-methyl-1H-imidazole-5-carbonyl)indolin-5-yl)thiazol-2-yl)acetamide (Cpd. No. 37)

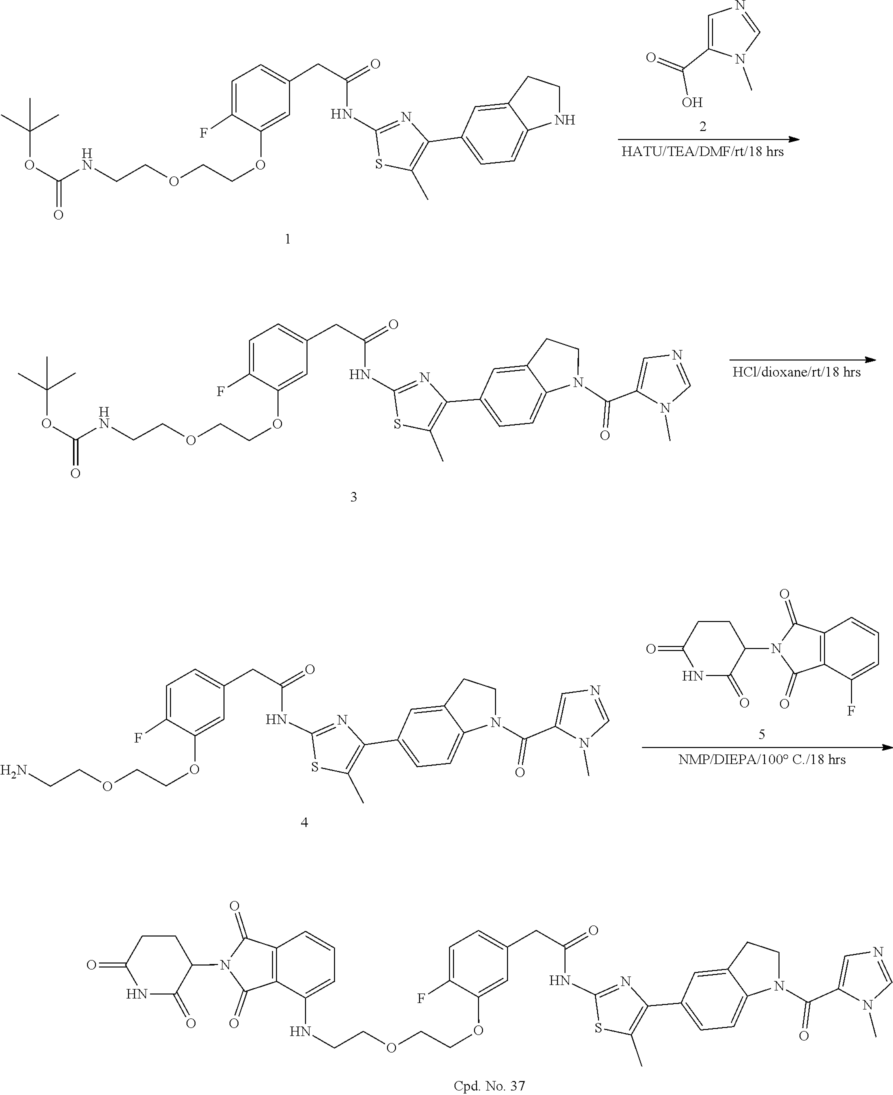

### Step 1. tert-butyl (2-(2-(2-fluoro-5-(2-((5-methyl-4-(1-(1-methyl-1H-imidazole-5-carbonyl)indolin-5-yl)thiazol-2-yl)amino)-2-oxoethyl)phenoxy)ethoxy)ethyl)carbamate

A solution of 1-methyl-1H-imidazole-5-carboxylic acid (0.17 g, 1.4 mmol), tert-butyl (2-(2-(2-fluoro-5-(2-((4-(indolin-5-yl)-5-methylthiazol-2-yl)amino)-2-oxoethyl)phenoxy)ethoxy)ethyl)carbamate (0.79 g, 1.4 mmol), 2-(7-azabenzotriazol-1-yl)-N,N,N′,N′-tetramethyluronium hexafluorophosphate (0.63 g, 1.7 mmol) and triethylamine (0.63 g, 1.7 mmol) in N,N-dimethylformamide (15 mL) was stirred at room temperature for 18 hrs. The mixture was diluted with water (20 mL), extracted with dichloromethane (25 mL×3), dried over anhydrous sodium sulfate, filtered and concentrated in vacuo. The residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15 min) to give tert-butyl (2-(2-(2-fluoro-5-(2-((5-methyl-4-(1-(1-methyl-1H-imidazole-5-carbonyl)indolin-5-yl)thiazol-2-yl)amino)-2-oxoethyl)phenoxy)ethoxy)ethyl)carbamate (0.62 g, 66% yield) as a white solid. MS (ESI) m/z: 679.2 [M+H]⁺.

### Step 2. 2-(3-(2-(2-aminoethoxy)ethoxy)-4-fluorophenyl)-N-(5-methyl-4-(1-(1-methyl-1H-imidazole-5-carbonyl)indolin-5-yl)thiazol-2-yl)acetamide

A solution of tert-butyl (2-(2-(2-fluoro-5-(2-((5-methyl-4-(1-(1-methyl-1H-imidazole-5-carbonyl)indolin-5-yl)thiazol-2-yl)amino)-2-oxoethyl)phenoxy)ethoxy)ethyl)carbamate (0.61 g, 0.90 mmol) in hydrochloric acid (1 M in 1,4-dioxane, 20 mL) was stirred at room temperature for 18 hrs. The solvent was removed in vacuo. The residue was dissolved in dichloromethane (80 mL), washed with saturated sodium bicarbonate aqueous solution, dried over anhydrous sodium sulfate, filtered and concentrated in vacuo. The residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15 min) to give 2-(3-(2-(2-aminoethoxy)ethoxy)-4-fluorophenyl)-N-(5-methyl-4-(1-(1-methyl-1H-imidazole-5-carbonyl)indolin-5-yl)thiazol-2-yl)acetamide (0.34 g, 65% yield) as a white solid. MS (ESI) m/z: 578.8 [M+H]⁺.

### Step 3. 2-(3-(2-(2-((2-(2,6-dioxopiperidin-3-yl)-1,3-dioxoisoindolin-4-yl)amino)ethoxy)ethoxy)-4-fluorophenyl)-N-(5-methyl-4-(1-(1-methyl-1H-imidazole-5-carbonyl)indolin-5-yl)thiazol-2-yl)acetamide

A solution of 2-(3-(2-(2-aminoethoxy)ethoxy)-4-fluorophenyl)-N-(5-methyl-4-(1-(1-methyl-1H-imidazole-5-carbonyl)indolin-5-yl)thiazol-2-yl)acetamide (0.33 g, 0.57 mmol), 2-(2,6-dioxopiperidin-3-yl)-4-fluoroisoindoline-1,3-dione (0.79 g, 2.9 mmol and N,N-diisopropylethylamine (0.44 g, 3.4 mmol) in 1-methylpyrrolidin-2-one (10 mL) was stirred at 100° C. for 18 hrs. The mixture was diluted with water (25 mL), extracted with dichloromethane (30 mL×3), dried over anhydrous sodium sulfate, filtered and concentrated in vacuo. The residue was purified by Prep-HPLC (Column: Welch Xtimate 21.2*250 mm C18, 10 μm, Mobile Phase: A: water (10 mmol/L ammonium bicarbonate) B: acetonitrile; B %: 30%-70% in 15.0 min) to give 2-(3-(2-(2-((2-(2,6-dioxopiperidin-3-yl)-1,3-dioxoisoindolin-4-yl)amino)ethoxy)ethoxy)-4-fluorophenyl)-N-(5-methyl-4-(1-(1-methyl-1H-imidazole-5-carbonyl)indolin-5-yl)thiazol-2-yl)acetamide (23.3 mg, 4.9% yield) as a yellow solid. ¹H NMR (400 MHz, DMSO-d₆): δ 12.31 (s, 1H), 11.10 (s, 1H), 8.07 (d, J=8.4 Hz, 1H), 7.85 (s, 1H), 7.59 (s, 1H), 7.56-7.52 (m, 2H), 7.47 (dd, J=8.0 Hz, 1.6 Hz, 1H), 7.17-7.12 (m, 3H), 7.01 (d, J=6.8 Hz, 1H), 6.89-6.86 (m, 1H), 6.62 (t, J=5.6 Hz, 1H), 5.04 (dd, J=12.8 I-z, 5.2 Hz, 1H), 4.36 (t, J=8.0 Hz, 2H), 4.17 (t, J=4.4 Hz, 2H), 3.82-3.80 (m, 5H), 3.70-3.68 (m, 3.49 (q, J=5.6 Hz, 2H), 3.19 (t, J=8.0 Hz, 2H), 2.92-2.87 (m, 1H), 2.59-2.50 (m, 2H), 2.44 (s, 3H), 2.03-1.96 (m, 1H). MS (ESI) m/z: 835.2 [M+H]⁺.

### Example 39

### Synthesis of N-(3-(2-(2-(2-(2,6-dioxopiperidin-3-yl)-1,3-dioxoisoindolin-5-ylamino)ethoxy)ethoxy)benzyl)-4-(1-(cyclopropanecarbonyl)indolin-5-yl)-5-methylthiazole-2-carboxamide (Cpd. No. 38)

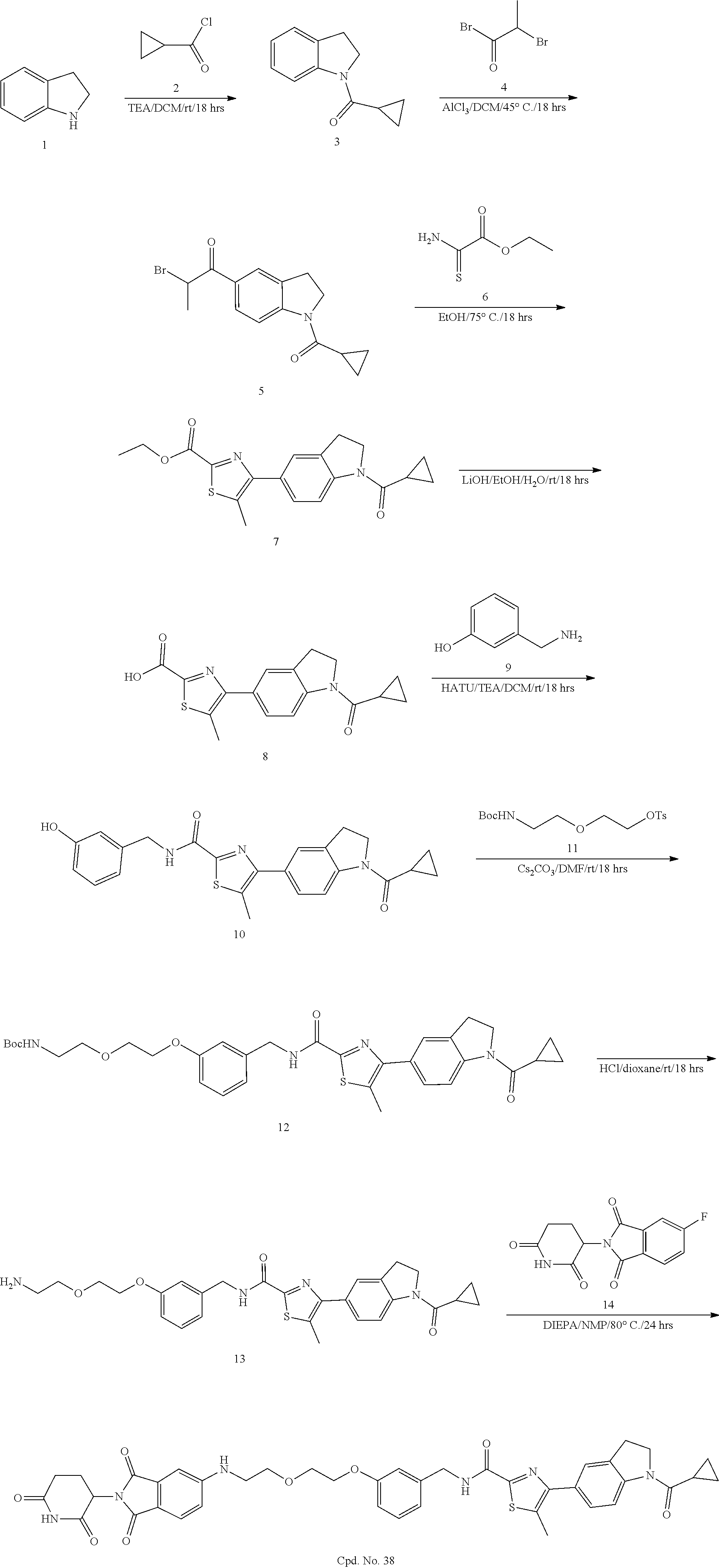

### Step 1. Cyclopropyl(indolin-1-yl)methanone

To a solution of indoline (10.00 g, 83.92 mmol) and triethylamine (21.23 g, 209.8 mmol) in dichloromethane (100 mL) was added cyclopropanecarbonyl chloride (10.53 g, 100.7 mmol) at 0° C. After addition, the mixture was stirred at room temperature for 18 hrs. The mixture was quenched with water (50 mL), extracted with dichloromethane (50 mL×3), washed with brine (50 mL×2) dried over anhydrous sodium sulfate, filtered and concentrated in vacuo to give cyclopropyl(indolin-1-yl)methanone (24.0 g, overweight) as a yellow solid. MS (ESI) m/z: 188.3 [M+H]⁺.

### Step 2. 2-bromo-1-(1-(cyclopropanecarbonyl)indolin-5-yl)propan-1-one

Cyclopropyl(indolin-1-yl)methanone (23.00 g, 80.4 mmol theoretical maximum) and aluminium chloride (29.48 g, 221.1 mmol) was dissolved in dichloromethane (600 mL), then 2-bromopropanoyl bromide (47.73 g, 221.1 mmol) was added. After addition, the mixture was stirred at 45° C. for 18 hrs. The mixture was cooled to room temperature, quenched with water (300 mL), basified to give pH==8-10 with sodium hydroxide aqueous solution, extracted with dichloromethane (400 mL), dried over anhydrous sodium sulfate, filtered and concentrated in vacuo to give crude 2-bromo-1-(1-(cyclopropanecarbonyl)indolin-5-yl)propan-1-one (34.0 g, overweight) as a yellow oil. MS (ESI) m/z: 322.0 and 324.0 [M+H]⁺.

### Step 3. ethyl 4-(1-(cyclopropanecarbonyl)indolin-5-yl)-5-methylthiazole-2-carboxylate

A solution of 2-bromo-1-(1-(cyclopropanecarbonyl)indolin-5-yl)propan-1-one (34.0 g, 80.4 mmol theoretical maximum) and ethyl 2-amino-2-thioxoacetate (28.11 g, 211 mmol) in ethanol (200 mL) was stirred at 75° C. for 18 hrs. The mixture was cooled to room temperature, diluted with water (1 L), filtered. The filter cake was washed with water (100 mL×2), dried to give ethyl 4-(1-(cyclopropanecarbonyl)indolin-5-yl)-5-methylthiazole-2-carboxylate (37.70 g, overweight) as yellow solid. MS (ESI) m/z: 357.1 [M+H]⁺.

### Step 4. 4-(1-(cyclopropanecarbonyl)indolin-5-yl)-5-methylthiazole-2-carboxylic acid

A solution of ethyl 4-(1-(cyclopropanecarbonyl)indolin-5-yl)-5-methylthiazole-2-carboxylate (36.00 g, 76.8 mmol theoretical maximum) and lithium hydroxide (7.26 g, 303 mmol) in ethanol (100 mL) and water (100 mL) was stirred at room temperature for 18 hrs. The ethanol was removed in vacuo and the residue was acidified with diluted hydrochloric acid aqueous solution, filtered. The filter cake was washed with water (30 mL×2), dried to give 4-(1-(cyclopropanecarbonyl)indolin-5-yl)-5-methylthiazole-2-carboxylic acid (21.0 g, 83.3% yield overall from Step 1) as yellow solid. MS (ESI) m/z: 328.9 [M+H]⁺.

### Step 5. N-(3-hydroxybenzyl)-4-(1-(cyclopropanecarbonyl)indolin-5-yl)-5-methylthiazole-2-carboxamide

A solution of 4-(1-(cyclopropanecarbonyl)indolin-5-yl)-5-methylthiazole-2-carboxylic acid (0.50 g, 1.5 mmol), 2-(7-azabenzotriazol-1-yl)-N,N,N′,N′-tetramethyluroniumhexafluorophosphate (1.16 g, 3.05 mmol) and triethylamine (0.46 g, 4.6 mmol) in dichloromethane (20 mL) was stirred at room temperature for 30 mins. 3-(aminomethyl)phenol (0.19 g, 1.5 mmol) was added into the resulting mixture. The mixture was stirred at room temperature for 18 hrs. The mixture was diluted with water (30 mL), extracted with dichloromethane (50 mL×2), washed with brine (20 mL), dried over anhydrous sodium sulfate, filtered and concentrated in vacuo. The residue was purified by flash column chromatography (dichloromethane/methanol=5/95) to give A-(3-hydroxybenzyl)-4-(1-(cyclopropanecarbonyl)indolin-5-yl)-5-methylthiazole-2-carboxamide (0.40 g, 61% yield) as a yellow solid. MS (ESI) m/z 434.2 [M+H]+

### Step 6. tert-butyl 2-(2-(3-((4-(1-(cyclopropanecarbonyl)indolin-5-yl)-5-methylthiazole-2-carboxamido)methyl)phenoxy)ethoxy)ethylcarbamate

A solution of N-(3-hydroxybenzyl)-4-(1-(cyclopropanecarbonyl)indolin-5-yl)-5-methylthiazole-2-carboxamide (0.40 g, 0.92 mmol), 2-(2-((tert-butoxycarbonyl)amino)ethoxy)ethyl 4-methylbenzenesulfonate (0.33 g, 0.92 mmol) and cesium carbonate (0.60 g, 1.85 mmol) in N,N-dimethylformamide (20 mL) was stirred at room temperature for 18 hrs. The mixture was quenched with water (35 mL), extracted with dichloromethane (40 mL×2), washed with brine (20 mL), dried over anhydrous sodium sulfate, filtered and concentrated in vacuo. The residue was purified by flash column chromatography (dichloromethane/methane=3/97) to give tert-butyl 2-(2-(3-((4-(1-(cyclopropanecarbonyl)indolin-5-yl)-5-methylthiazole-2-carboxamido)methyl)phenoxy)ethoxy)ethylcarbamate (0.30 g, 52% yield) as a yellow solid. MS (ESI) m/z 621.0 [M+H]⁺

### Step 7. N-(3-(2-(2-aminoethoxy)ethoxy)benzyl)-4-(1-(cyclopropanecarbonyl)indolin-5-yl)-5-methylthiazole-2-carboxamide

A solution of tert-butyl 2-(2-(3-((4-(1-(cyclopropanecarbonyl)indolin-5-yl)-5-methylthiazole-2-carboxamido)methyl)phenoxy)ethoxy)ethylcarbamate (0.30 g, 0.48 mmol) in hydrochloric acid (2 M in 1,4-dioxane, 10 mL) and 1,4-dioxane (5 mL) was stirred at room temperature for 18 hrs. The mixture was concentrated in vacuo. The residue was dissolved in dichloromethane (80 mL), washed with saturated sodium bicarbonate aqueous solution, dried over anhydrous sodium sulfate, filtered and concentrated in vacuo to give N-(3-(2-(2-aminoethoxy)ethoxy)benzyl)-4-(1-(cyclopropanecarbonyl)indolin-5-yl)-5-methylthiazole-2-carboxamide (0.16 g, 64% yield) as a white solid. MS (ESI) m/z 521.0 [M+H]⁺

### Step 8. N-(3-(2-(2-(2-(2,6-dioxopiperidin-3-yl)-1,3-dioxoisoindolin-5-ylamino)ethoxy)ethoxy)benzyl)-4-(1-(cyclopropanecarbonyl)indolin-5-yl)-5-methylthiazole-2-carboxamide

A solution of N-(3-(2-(2-aminoethoxy)ethoxy)benzyl)-4-(1-(cyclopropanecarbonyl)indolin-5-yl)-5-methylthiazole-2-carboxamide (0.16 g, 0.31 mmol), 2-(2,6-dioxopiperidin-3-yl)-5-fluoroisoindoline-1,3-dione (0.36 g, 1.3 mmol) and N, N-diisopropylethylamine (0.17 g, 1.3 mmol) in 1-methylpyrrolidin-2-one (5 mL) was stirred at 80° C. for 24 hrs. The mixture was cooled to room temperature, diluted with water (10 mL), extracted with dichloromethane (15 mL×2), dried over anhydrous sodium sulfate, filtered and concentrated in vacuo. The residue was purified by Prep-HPLC (Column: Welch Xtimate 21.2*250 mm C18, 10 μm, Mobile Phase: A: water (10 mmol/L ammonium bicarbonate) B: acetonitrile; B %: 30%-70% in 15 min) to give N-(3-(2-(2-(2-(2,6-dioxopiperidin-3-yl)-1,3-dioxoisoindolin-5-ylamino)ethoxy)ethoxy)benzyl)-4-(1-(cyclopropanecarbonyl)indolin-5-yl)-5-methylthiazole-2-carboxamide (6.7 mg, 2.8% yield) as a yellow solid. ¹H NMR (500 MHz, DMSO-d₆): δ 11.05 (s, 1H), 9.78 (t, J=6.5 Hz, 1H), 8.09 (d, J=7.0 Hz, 1H), 7.65 (s, 1H), 7.54 (d, J=8.0 Hz, 2H), 7.22 (t, J=8.5 Hz, 1H), 7.17 (t, J=6.0 Hz, 1H), 6.99 (m, 1H), 6.90-6.87 (m, 3H), 6.81 (dd, J=8.5 Hz, 2.0 Hz, 1H), 5.02 (dd, J=12.5 Hz, 5.5 Hz, 1H), 4.43 (d, J=6.5 Hz, 2H), 4.34 (m, 2H), 4.08 (t, J=3.5 Hz, 2H), 3.76 (t, J=4.5 Hz, 2H), 3.65 (t, J=6.0 Hz, 2H), 3.37 (dd, J=10.5 Hz, 5.0 Hz, 2H), 3.23 (t, J=7.5 Hz, 2H), 2.89-2.84 (m, 1H), 2.63-2.54 (m, 5H), 2.00-1.96 (m, 2H), 0.89-0.86 (m, 4H). MS (ESI) m/z: 776.8 [M+H]⁺.

### Example 40

### Synthesis of 4-(1-(cyclopropanecarbonyl)indolin-5-yl)-N-(3-(2-(2-((2-(2,6-dioxopiperidin-3-yl)-1,3-dioxoisoindolin-5-yl)amino)ethoxy)ethoxy)phenyl)-5-methylthiazole-2-carboxamide (Cpd. No. 39)

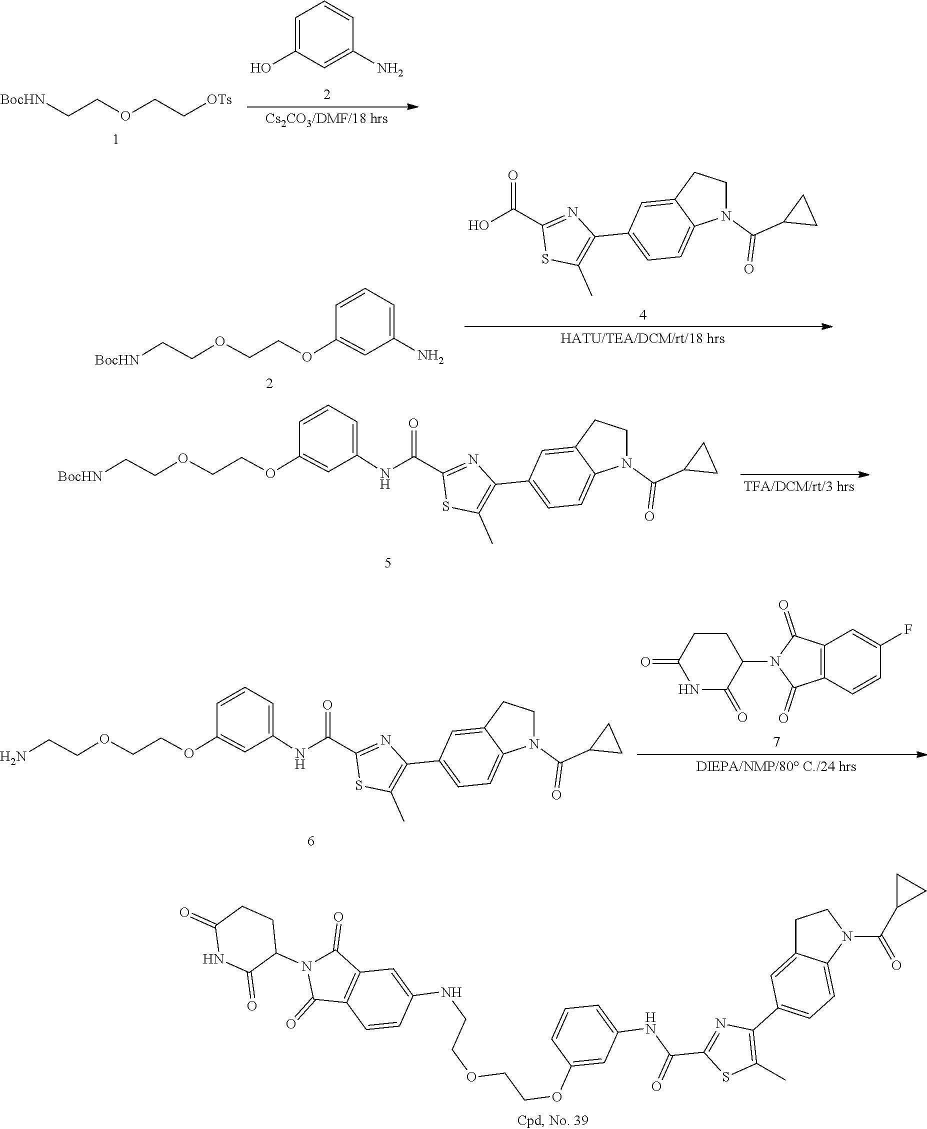

### Step 1. tert-butyl (2-(2-(3-aminophenoxy)ethoxy)ethyl)carbamate

A solution of 2-(2-((tert-butoxycarbonyl)amino)ethoxy)ethyl 4-methylbenzenesulfonate (1.00 g, 2.78 mmol), 3-aminophenol (0.30 g, 2.8 mmol) and cesium carbonate (1.81 g, 5.56 mmol) in N,N-dimethylformamide (20 mL) was stirred at room temperature for 18 hrs. The reaction was quenched with water (30 mL), extracted with dichloromethane (30 mL×2), dried over anhydrous sodium sulfate, filtered and concentrated in vacuo. The residue was purified by flash column chromatography (ethyl acetate/petroleum ether=1/2) to give tert-butyl (2-(2-(3-aminophenoxy)ethoxy)ethyl)carbamate (0.50 g, 61% yield) as a white solid. MS (ESI) m/z: 297.3 [M+H]⁺

### Step 2. tert-butyl (2-(2-(3-(4-(1-(cyclopropanecarbonyl)indolin-5-yl)-5-methylthiazole-2-carboxamido)phenoxy)ethoxy)ethyl)carbamate

A solution of tert-butyl (2-(2-(3-aminophenoxy)ethoxy)ethyl)carbamate (0.50 g, 1.69 mmol), 4-(1-(cyclopropanecarbonyl)indolin-5-yl)-5-methylthiazole-2-carboxylic acid (0.55 g, 1.69 mmol), 2-(7-azabenzotriazol-1-yl)-N,N,N′,N′-tetramethyluronium hexafluorophosphate (0.96 g, 2.5 mmol) and triethylamine (0.68 g, 6.8 mmol) in N,N-dimethylformamide (25 mL) was stirred at room temperature for 18 hrs. The reaction was quenched with water (20 mL), extracted with dichloromethane (25 mL×3), washed with brine (20 mL), dried over anhydrous sodium sulfate, filtered and concentrated in vacuo. The residue was purified by Pre-HPLC (Column: Welch Xtimate 21.2*250 mm C18, 10 μm, Mobile Phase: A: water (10 mmol/L ammonium bicarbonate) B: acetonitrile; B %: 30%-70% in 15 min) to give tert-butyl (2-(2-(3-(4-(1-(cyclopropanecarbonyl)indolin-5-yl)-5-methylthiazole-2-carboxamido)phenoxy)ethoxy)ethyl)carbamate (0.50 g, 49% yield) as a white solid. MS (ESI) m/z: 607.9 [M+H]⁺.

### Step 3. N-(3-(2-(2-aminoethoxy)ethoxy)phenyl)-4-(1-(cyclopropanecarbonyl)indolin-5-yl)-5-methylthiazole-2-carboxamide

A solution of tert-butyl (2-(2-(3-(4-(1-(cyclopropanecarbonyl)indolin-5-yl)-5-methylthiazole-2-carboxamido) phenoxy)ethoxy)ethyl)carbamate (0.50 g, 0.82 mmol) in trifluoroacetic acid (20 mL) and dichloromethane (20 mL) was stirred at room temperature for 3 hrs. The solvent was removed in vacuo, and then the residue was dissolved in dichloromethane (100 mL), washed with saturated sodium bicarbonate aqueous solution, dried over anhydrous sodium sulfate, filtered and concentrated in vacuo to give N-(3-(2-(2-aminoethoxy)ethoxy)phenyl)-4-(1-(cyclopropanecarbonyl)indolin-5-yl)-5-methylthiazole-2-carboxamide (0.40 g, 96% yield) as a yellow solid. MS (ESI) m/z: 507.3 [M+H]⁺.

### Step 4. 4-(1-(cyclopropanecarbonyl)indolin-5-yl)-N-(3-(2-(2-((2-(2,6-dioxopiperidin-3-yl)-1,3-dioxoisoindolin-5-yl)amino)ethoxy)ethoxy)phenyl)-5-methylthiazole-2-carboxamide

A solution of N-(3-(2-(2-aminoethoxy)ethoxy)phenyl)-4-(1-(cyclopropanecarbonyl)indolin-5-yl)-5-methylthiazole-2-carboxamide (0.30 g, 0.59 mmol), 2-(2,6-dioxopiperidin-3-yl)-5-fluoroisoindoline-1,3-dione (0.82 g, 3.0 mmol) and N, N-diisopropylethylamine (0.61 g, 4.7 mmol) in 1-methylpyrrolidin-2-one (25 mL) was stirred at 80° C. for 24 hrs. The mixture was cooled to room temperature, diluted with water (35 mL), extracted with dichloromethane (40 mL×2), washed with brine (20 mL), dried over anhydrous sodium sulfate, filtered and concentrated in vacuo. The residue was purified by Prep-HPLC (Column: Welch Xtimate 21.2*250 mm C18, 10 μm, Mobile Phase: A: water (10 mmol/L ammonium bicarbonate) B: acetonitrile; B %: 30%-70% in min) to give 4-(1-(cyclopropanecarbonyl)indolin-5-yl)-N-(3-(2-(2-((2-(2,6-dioxopiperidin-3-yl)-1,3-dioxoisoindolin-5-yl)amino)ethoxy)ethoxy)phenyl)-5-methylthiazole-2-carboxamide (42.1 mg, 9.3% yield) as a yellow solid. ¹H NMR (400 MHz, DMSO-d₆): δ 11.09 (brs, 1H), 10.42 (s, 1H), 8.10 (m, 1H), 7.72 (s, 1H), 7.66-7.53 (m, 3H), 7.46-7.39 (m, 1H), 7.27-7.21 (m, 2H), 6.96 (s, 1H), 6.87 (d, J=7.6 Hz, 1H), 6.71 (d, J=7.6 Hz, 1H), 5.02 (dd, J=12.4 Hz, 5.2 Hz, 1H), 4.36 (s, 2H), 4.05 (s, 2H), 3.68 (s, 2H), 3.60 (s, 2H), 3.39 (m, 2H), 3.26 (m, 2H), 2.90-2.79 (m, 1H), 2.63-2.56 (m, 5H), 1.98 (brs, 2H), 0.89 (brs, 4H). MS (ESI) m/z: 763.0 [M+H]⁺.

### Example 41

### Synthesis of N-(4-(1-(cyclopropanecarbonyl)indolin-5-yl)-5-methylthiazol-2-yl)-2-(3-(5-(2-(2,6-dioxopiperidin-3-yl)-1,3-dioxoisoindolin-4-ylamino)pentyloxy)phenyl)acetamide (Cpd. No. 40)

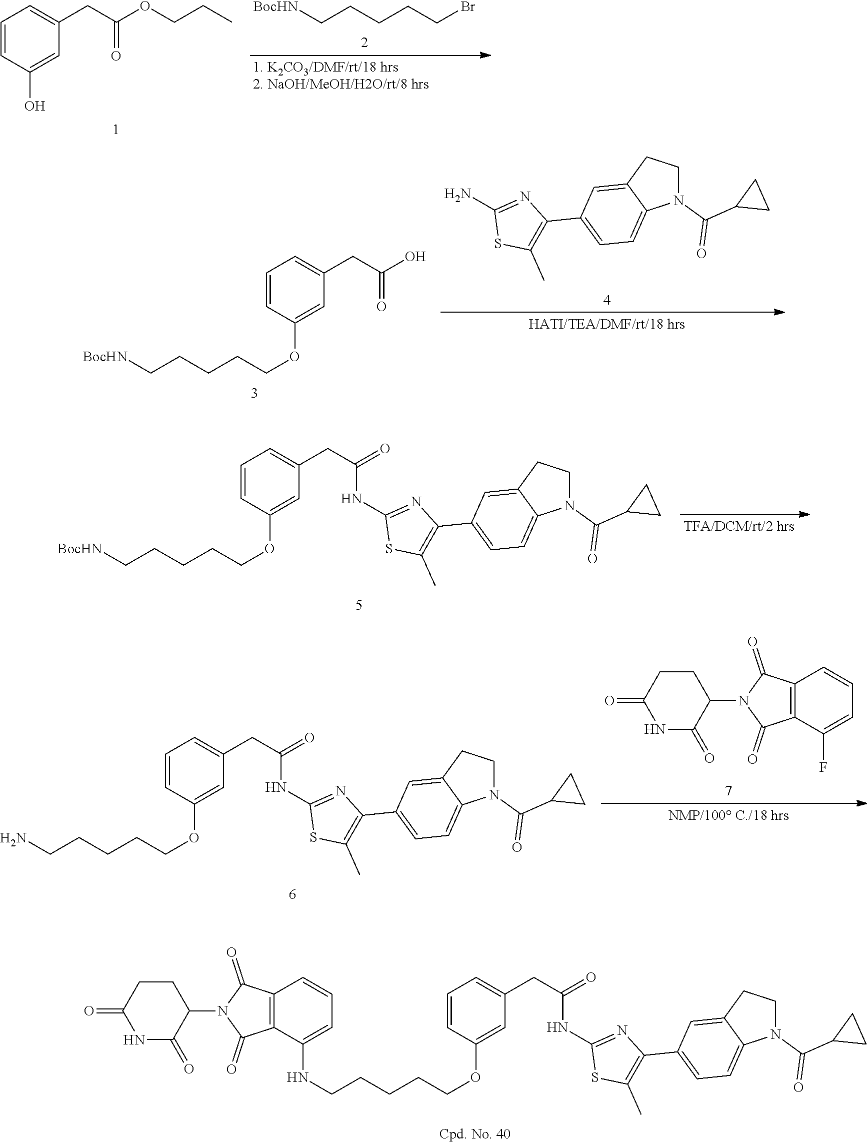

### Step 1. 2-(3-((5-((tert-butoxycarbonyl)amino)pentyl)oxy)phenyl)acetic acid

A mixture of ethyl 2-(3-hydroxyphenyl)acetate (1.00 g, 5.55 mmol), tert-butyl 5-bromopentylcarbamate (1.77 g, 6.65 mmol) and potassium carbonate (2.30 g, 16.7 mmol) in N,N-dimethylformamide (20 mL) was stirred at room temperature for 18 hrs. Sodium hydroxide (1.11 g, 27.8 mmol), methanol (20 mL) and water (10 mL) were added into the resulting mixture, and then the mixture was stirred at room temperature for 8 hrs. The mixture was diluted with water (30 mL), acidified with hydrochloric acid (2 M) to pH=3-4, extracted with dichloromethane (40 mL×3), washed with brine (30 mL×2), dried over anhydrous sodium sulfate, filtered and concentrated in vacuo. The residue was purified by flash column chromatography (ethyl acetate/petroleum ether=1/3) to give 2-(3-(5-(tert-butoxycarbonyl)pentyloxy)phenyl)acetic acid (0.71 g, 38% yield) as a white solid. MS (ESI) m/z: 238.0 [MH-Boc]⁺.

### Step 2. tert-butyl 5-(3-(2-(4-(1-(cyclopropanecarbonyl)indolin-5-yl)-5-methylthiazol-2-ylamino)-2-oxoethyl)phenoxy)pentylcarbamate

A mixture of 2-(3-((5-((tert-butoxycarbonyl)amino)pentyl)oxy)phenyl)acetic acid (0.60 g, 1.78 mmol), (5-(2-amino-5-methylthiazol-4-yl)indolin-1-yl)(cyclopropyl)methanone (1.33 g, 4.45 mmol), triethylamine (0.90 g, 8.9 mmol) and 2-(7-azabenzotriazol-1-yl)-N,N,N′,N′-tetramethyluronium hexafluorophosphate (1.69 g, 4.45 mmol) in N,N-dimethylformamide (20 mL) was stirred at room temperature for 18 hrs. The reaction was quenched with water (20 mL), extracted with dichloromethane (30 mL×3), washed with brine (20 mL×2), dried over anhydrous sodium sulfate, filtered and concentrated in vacuo. The residue was purified by flash column chromatography (ethyl acetate) to give tert-butyl 5-(3-(2-(4-(1-(cyclopropanecarbonyl)indolin-5-yl)-5-methylthiazol-2-ylamino)-2-oxoethyl)phenoxy)pentylcarbamate (0.80 g, 73% yield) as a yellow solid. MS (ESI) m/z: 619.0 [M+H]⁺.

### Step 3. 2-(3-(5-aminopentyloxy)phenyl)-N-(4-(1-(cyclopropanecarbonyl)indolin-5-yl)-5-methyl thiazol-2-yl)acetamide

A solution of tert-butyl 5-(3-(2-(4-(1-(cyclopropanecarbonyl)indolin-5-yl)-5-methylthiazol-2-ylamino)-2-oxoethyl)phenoxy)pentylcarbamate (0.38 g, 0.61 mmol) and trifluoroacetic acid (15 mL) in dichloromethane (10 mL) was stirred at room temperature for 2 hrs. The solvent was removed in vacuo. The residue was dissolved in dichloromethane (100 mL), washed with saturated sodium carbonate aqueous solution, dried over anhydrous sodium carbonate, filtered and concentrated in vacuo to give 2-(3-(5-aminopentyloxy)phenyl)-N-(4-(1-(cyclopropanecarbonyl)indolin-5-yl)-5-methylthiazol-2-yl)acetamide (030 g, 94% yield) as a yellow solid. MS (ESI) m/z: 519.2 [M+H]⁺.

### Step 4. N-(4-(1-(cyclopropanecarbonyl)indolin-5-yl)-5-methylthiazol-2-yl)-2-(3-(5-(2-(2,6-dioxopiperidin-3-yl)-1,3-dioxoisoindolin-4-ylamino)pentyloxy)phenyl)acetamide

A solution of 2-(3-(5-aminopentyloxy)phenyl)-N-(4-(1-(cyclopropanecarbonyl)indolin-5-yl)-5-methylthiazol-2-yl) acetamide (0.14 g, 0.27 mmol), 2-(2,6-dioxopiperidin-3-yl)-4-fluoroisoindoline-1,3-dione (0.37 g, 1.3 mmol) and N,N-diisopropylethylamine (0.17 g, 1.3 mmol) in 1-methylpyrrolidin-2-one (5 mL) was stirred at 100° C. for 18 hrs. The mixture was cooled to room temperature, diluted with water (10 mL), extracted with dichloromethane (20 mL×3), washed with brine (20 mL), dried over anhydrous sodium sulfate, filtered and concentrated in vacuo. The residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15 min) to give N-(4-(1-(cyclopropanecarbonyl)indolin-5-yl)-5-methylthiazol-2-yl)-2-(3-(5-(2-(2,6-dioxopiperidin-3-yl)-1,3-dioxoisoindolin-4-ylamino)pentyloxy)phenyl)acetamide (65.4 mg, 31% yield) as a yellow solid. ¹H NMR (500 MHz, DMSO-d₆): δ 12.27 (s, 1H), 11.08 (s, 1H), 8.06 (d, J=7.0 Hz, 1H), 7.56 (t, J=8.0 Hz, 1H), 7.49 (s, 1H), 7.41 (d, J=8.0 Hz, 1H), 7.21 (t, J=8.0 z, 1H), 7.09 (d, J=9.0 Hz, 1H), 7.01 (d, J=7.5 Hz, 1H), 6.90 (s, 1H), 6.87 (d, J=7.5 Hz, 1H), 6.81 (d, J=8.5 Hz, 1H), 6.56 (t, J=6.0 Hz, 1H), 5.03 (dd, J=12.5 Hz, 5.5 Hz, 1H), 4.32 (brs, 2H), 3.96 (t, J=6.5 Hz, 2H), 3.69 (s, 2H), 3.30 (s, 2H), 3.22 (t, J=8.0 Hz, 2H), 2.88-2.84 (m, 1H), 2.59-2.52 (m, 2H), 2.43 (s, 3H), 2.03-1.98 (m, 2H), 1.77-1.73 (m, 2H), 1.65-1.61 (m, 2H), 1.51-1.48 (m, 2H), 0.88-0.86 (m, 4H). MS (ESI) m/z: 775.3 [M+H]⁺.

### Example 42

### Synthesis of N-(4-(1-(cyclopropanecarbonyl)indolin-5-yl)-5-methylthiazol-2-yl)-2-(3-(5-(2-(2,6-dioxopiperidin-3-yl)-1,3-dioxoisoindolin-4-ylamino)-3-methylpentyloxy)phenyl)acetamide (Cpd. No. 41)

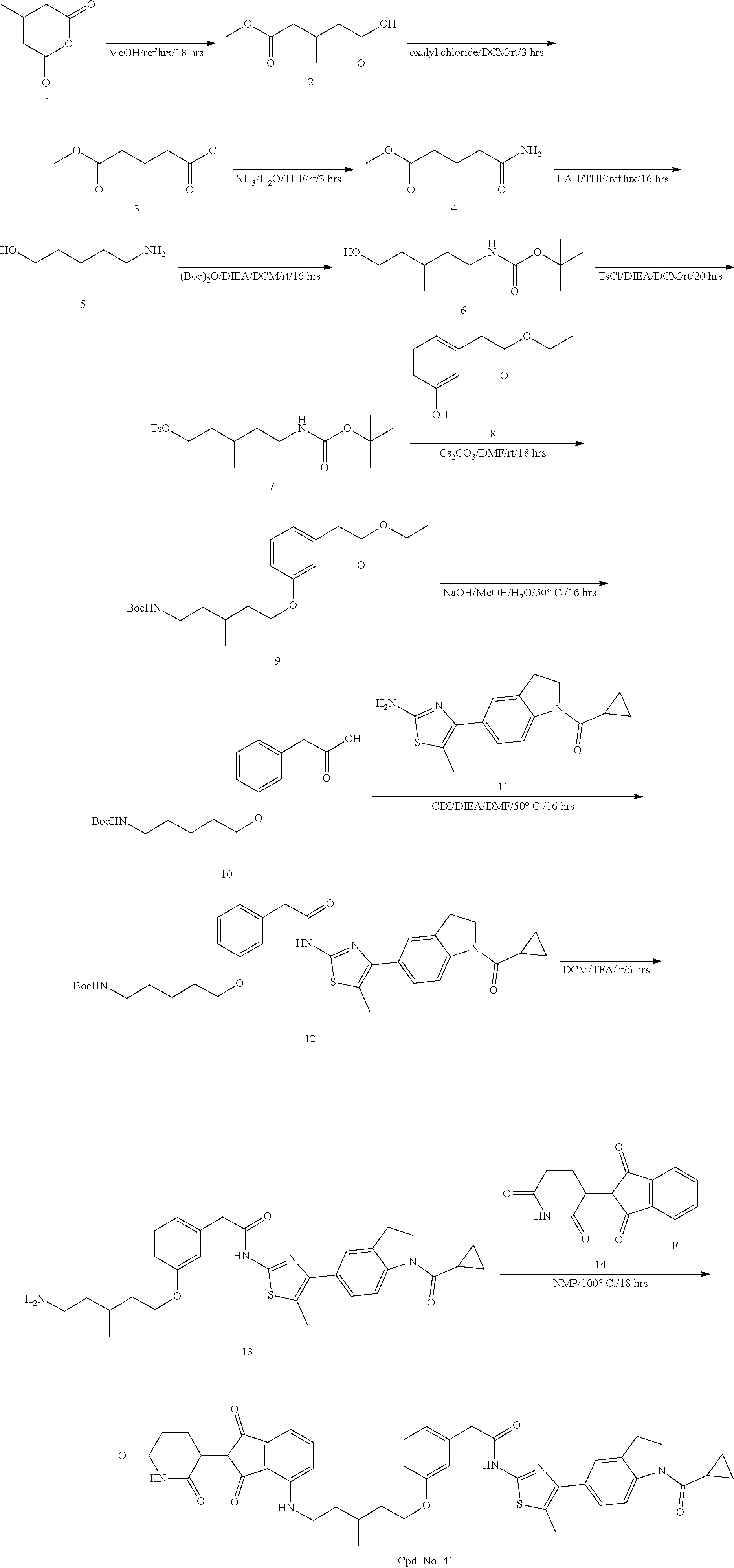

### Step 1. 5-methoxy-3-methyl-5-oxopentanoic acid

A solution of 4-methyl-dihydro-31-pyran-2,6-dione (15.00 g, 117.1 mmol) in methanol (100 mL) was stirred at 60° C. for 18 hrs. The solvent was removed in vacuo to give 5-methoxy-3-methyl-5-oxopentanoic acid (13.00 g, 69.3% yield) as a colorless oil. MS (ESI) m/z: 161.1 [M+H]⁺.

### Step 2. methyl 5-chloro-3-methyl-5-oxopentanoate

To a solution of 5-methoxy-3-methyl-5-oxopentanoic acid (10.00 g, 62.43 mmol) and N,N-dimethylfornamide (0.1 mL) in dichloromethane (100 mL) was added oxalyl chloride (8.32 g, 65.6 mmol) at 0° C. After addition, the mixture was stirred at room temperature for 3 hrs. The solvent was removed in vacuo to give methyl 5-chloro-3-methyl-5-oxopentanoate (8.00 g, 71.7% yield) as a yellow oil.

### Step 3. methyl 5-amino-3-methyl-5-oxopentanoate

To a solution of ammonium hydroxide (40 mL) in tetrahydrofuran (30 mL) was added methyl 5-chloro-3-methyl-5-oxopentanoate (8.00 g, 44.8 mmol) at 0° C. After addition, the mixture was stirred at room temperature for 3 hrs. The mixture was diluted with water (50 mL), extracted with dichloromethane (80 mL×3), dried over anhydrous sodium sulfate, filtered and concentrated in vacuo to give methyl 5-amino-3-methyl-5-oxopentanoate (5.00 g, 70.1% yield) as a yellow oil. MS (ESI) m/z: 160.1 [M+H]⁺.

### Step 4. 5-amino-3-methylpentan-1-ol

To a solution of methyl 5-amino-3-methyl-5-oxopentanoate (4.90 g, 30.8 mmol) in tetrahydrofuran (100 mL), was added lithium aluminum hydride (3.50 g, 92.3 mmol) at 0° C. After addition, the mixture was stirred at 50° C. for 16 hrs. The mixture was cooled to room temperature, quenched with water, filtered. The filter cake was washed with dichloromethane (50 mL×3), then the combined filtrate was dried over anhydrous sodium, filtered and concentrated to give 5-amino-3-methylpentan-1-ol (3.00 g, 83.2% yield) as yellow oil. MS (ESI) m/z: 118.2 [M+H]⁺.

### Step 5. tert-butyl (5-hydroxy-3-methylpentyl)carbamate

A solution of 5-amino-3-methylpentan-1-ol (2.90 g, 24.7 mmol), di-tert-butyl dicarbonate (6.48 g, 29.7 mmol) and N,N-diisopropylethylamine (9.59 g, 74.2 mmol) in dichloromethane (50 mL) was stirred at room temperature for 16 hrs. The mixture was diluted with water (90 mL), extracted with dichloromethane (80 mL×3), washed with brine (30 mL), dried over anhydrous sodium sulfate, filtered and concentrated in vacuo. The residue was purified by flash column chromatography (ethyl acetate/petroleum ether=1/3) to give tert-butyl (5-hydroxy-3-methylpentyl)carbamate (2.00 g, 37.2% yield) as a white solid. MS (ESI) m/z: 218.1 [M+H]⁺.

### Step 6. 5-((tert-butoxycarbonyl)amino)-3-methylpentyl 4-methylbenzenesulfonate

A solution tert-butyl (5-hydroxy-3-methylpentyl)carbamate (1.90 g, 8.74 mmol), tosyl chloride (2.00 g, 10.5 mmol) and N,N-diisopropylethylamine (3.39 g, 26.2 mmol) in dichloromethane (60 mL) was stirred at room temperature for 20 hrs. The mixture was diluted with water (80 mL), extracted with dichloromethane (60 mL×3), washed with brine (30 mL), dried over anhydrous sodium sulfate, filtered and concentrated in vacuo. The residue was purified by flash column chromatography (ethyl acetate/petroleum ether=1/4) to give 5-((tert-butoxycarbonyl)amino)-3-methylpentyl-4-methylbenzenesulfonate (1.30 g, 40.0% yield) as a yellow solid. MS (ESI) m/z: 271.8 [M+H−100]⁺.

### Step 7. ethyl 2-(3-((5-((tert-butoxycarbonyl)amino)-3-methylpentyl)oxy)phenyl)acetate

A solution of 5-((tert-butoxycarbonyl)amino)-3-methylpentyl-4-methylbenzenesulfonate (1.20 g, 3.23 mmol), ethyl 2-(3-hydroxyphenyl)acetate (0.76 g, 4.2 mmol) and cesium carbonate (3.16 g, 9.69 mmol) in N,N-dimethylformamide (20 mL) was stirred at room temperature for 18 hours. The mixture was diluted with water (45 mL), extracted with dichloromethane (50 mL×3), washed with brine (40 mL), dried over anhydrous sodium sulfate, filtered and concentrated in vacuo. The residue was purified by flash column chromatography (ethyl acetate/petroleum ether=I/3) to give ethyl 2-(3-((5-((tert-butoxycarbonyl)amino)-3-methylpentyl) oxy)phenyl)acetate (0.50 g, 41% yield) as yellow solid. MS (ESI) m/z: 280.1 [M+H−100]⁺.

### Step 8. 2-(3-((5-((tert-butoxycarbonyl)amino)-3-methylpentyl)oxy)phenyl)acetic acid

A solution of ethyl 2-(3-((5-((tert-butoxycarbonyl)amino)-3-methylpentyl)oxy)phenyl)acetate (0.40 g, 1.05 mmol) and sodium hydroxide (0.17 g, 4.2 mmol) in methanol (25 mL) and water (10 mL) was stirred at 50° C. for 16 hrs. The mixture was cooled to room temperature, acidified with aqueous hydrochloric acid solution (3 M) to pH=4, extracted with dichloromethane (40 mL×3), washed with brine (30 mL), dried over anhydrous sodium sulfate, filtered and concentrated in vacuo to give 2-(3-((5-((tert-butoxycarbonyl)amino)-3-methylpentyl)oxy)phenyl)acetic acid (0.30 g, 81% yield) as a white solid. MS (ESI) mi/z: 252.1 [M+H−100]⁺.

### Step 9. tert-butyl (5-(3-(2-((4-(1-(cyclopropanecarbonyl)indolin-5-yl)-5-methylthiazol-2-yl)amino)-2-oxoethyl)phenoxy)-3-methylpentyl)carbamate

A solution of 2-(3-((5-((tert-butoxycarbonyl)amino)-3-methylpentyl)oxy)phenyl)acetic acid (0.25 g, 0.71 mmol), N,N-diisopropylethylamine (0.28 g, 2.1 mmol) and 1,1′-carbonyldiimidazole (0.16 g, 1.0 mmol) in N,N-dimethylformamide (15 mL) was stirred at room temperature for 1 hr. (5-(2-amino-5-methylthiazol-4-yl)indolin-1-yl)(cyclopropyl)methanone (0.26 g, 0.85 mmol) was added into the resulting mixture. The mixture was stirred at 50° C. for 16 hours. The mixture was diluted with water (30 mL), extracted with dichloromethane (20 mL×3), dried over anhydrous sodium sulfate, filtered and concentrated in vacuo. The residue was purified by flash column chromatography (methanol/dichloromethane=1/10) to give tert-butyl (5-(3-(2-((4-(1-(cyclopropanecarbonyl)indolin-5-yl)-5-methylthiazol-2-yl)amino)-2-oxoethyl)phenoxy)-3-methylpentyl)carbamate (0.18 g, 40% yield) as a white solid. MS (ESI) m/z: 633.1 [M+H]⁺.

### Step 10. 2-(3-((5-amino-3-methylpentyl)oxy)phenyl)-N-(4-(1-(cyclopropanecarbonyl)indolin-5-yl)-5-methylthiazol-2-yl)acetamide

A solution of tert-butyl (5-(3-(2-((4-(1-(cyclopropanecarbonyl)indolin-5-yl)-5-methylthiazol-2-yl)amino)-2-oxoethyl)phenoxy)-3-methylpentyl)carbamate (0.18 g, 0.28 mmol) and trifluoroacetic acid (10 mL) in dichloromethane (10 mL) was stirred at room temperature for 6 hrs. The solvent was removed in vacuo and the residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15 min) to give 2-(3-((5-amino-3-methylpentyl)oxy)phenyl)-N-(4-(1-(cyclopropanecarbonyl)indolin-5-yl)-5-methylthiazol-2-yl)acetamide (60.0 mg, 40% yield) as a white solid. MS (ESI) m/z: 533.2 [M+H]⁺.

### Step 11. N-(4-(1-(cyclopropanecarbonyl)indolin-5-yl)-5-methylthiazol-2-yl)-2-(3-((5-((2-(2,6-dioxopiperidin-3-yl)-1,3-dioxoisoindolin-4-yl)amino)-3-methylpentyl)oxy)phenyl)acetamide

A solution of 2-(3-((5-amino-3-methylpentyl)oxy)phenyl)-AN-(4-(1-(cyclopropanecarbonyl)indolin-5-yl)-5-methylthiazol-2-yl)acetamide (55.0 mg, 0.10 mmol), 2-(2,6-dioxopiperidin-3-yl)-4-fluoroisoindoline-1,3-dione (57.0 mg, 0.21 mmol) and N, N-diisopropylethylamine (40.0 mg, 0.31 mmol) in 1-methylpyrrolidin-2-one (2 mL) was stirred at 100° C. for 18 hrs. The mixture was diluted with water (10 mL), extracted with dichloromethane (30 mL×3), washed with brine (20 mL), dried over anhydrous sodium sulfate, filtered and concentrated in vacuo. The residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15 min) to give N-(4-(1-(cyclopropanecarbonyl)indolin-5-yl)-5-methylthiazol-2-yl)-2-(3-((5-((2-(2,6-dioxopiperidin-3-yl)-1,3-dioxoisoindolin-4-yl)amino)-3-methylpentyl)oxy)phenyl)acetamide (14.7 mg, 18.0% yield) as a yellow solid.

¹H NMR (400 MHz, DMSO-d₆): δ 12.28 (s, 1H), 11.09 (s, 1H), 8.07 (d, J=7.6 Hz, 1H), 7.55 (t, J=8.4 Hz, 1H), 7.49 (s, 1H), 7.42 (d, J=7.6 Hz, 1H), 7.21 (t, J=8.0 Hz, 1H), 7.07 (d, J=8.8 Hz, 1H), 7.00 (d, J=7.2 Hz, 1H), 6.91 (s, 1H), 6.87 (d, J=8.0 Hz, 1H), 6.82 (dd, J=8.2 Hz, 2.4 Hz, 1H), 6.53 (t, J=5.2 Hz, 1H), 5.04 (dd, J=12.4 Hz, 4.8 Hz, 1H), 4.32-4.30 (m, 2H), 4.00-3.97 (m, 2H), 3.69 (s, 2H), 3.37-3.35 (m, 2H), 3.24-3.20 (m, 2H), 2.87-2.84 (m, 1H), 2.60-2.55 (m, 1H), 2.51-2.49 (in 1H), 2.42 (s, 3H), 2.03-1.95 (m, 2H), 1.95-1.79 (m, 2H), 1.78-1.65 (m, 2H), 1.60-1.42 (m, 1H), 0.99 (d, J=6.4 Hz, 3H), 0.87-0.84 (m, 4H). MS (ESI) m/z: 788.8 [M+H]⁺.

### Example 43

### Synthesis of N-(4-(1-(cyclopropanecarbonyl)indolin-5-yl)-5-methylthiazol-2-yl)-2-(3-(5-(2-(2,6-dioxopiperidin-3-yl)-1,3-dioxoisoindolin-4-ylamino)-3,3-dimethylpentyloxy)phenyl)acetamide (Cpd. No. 42)

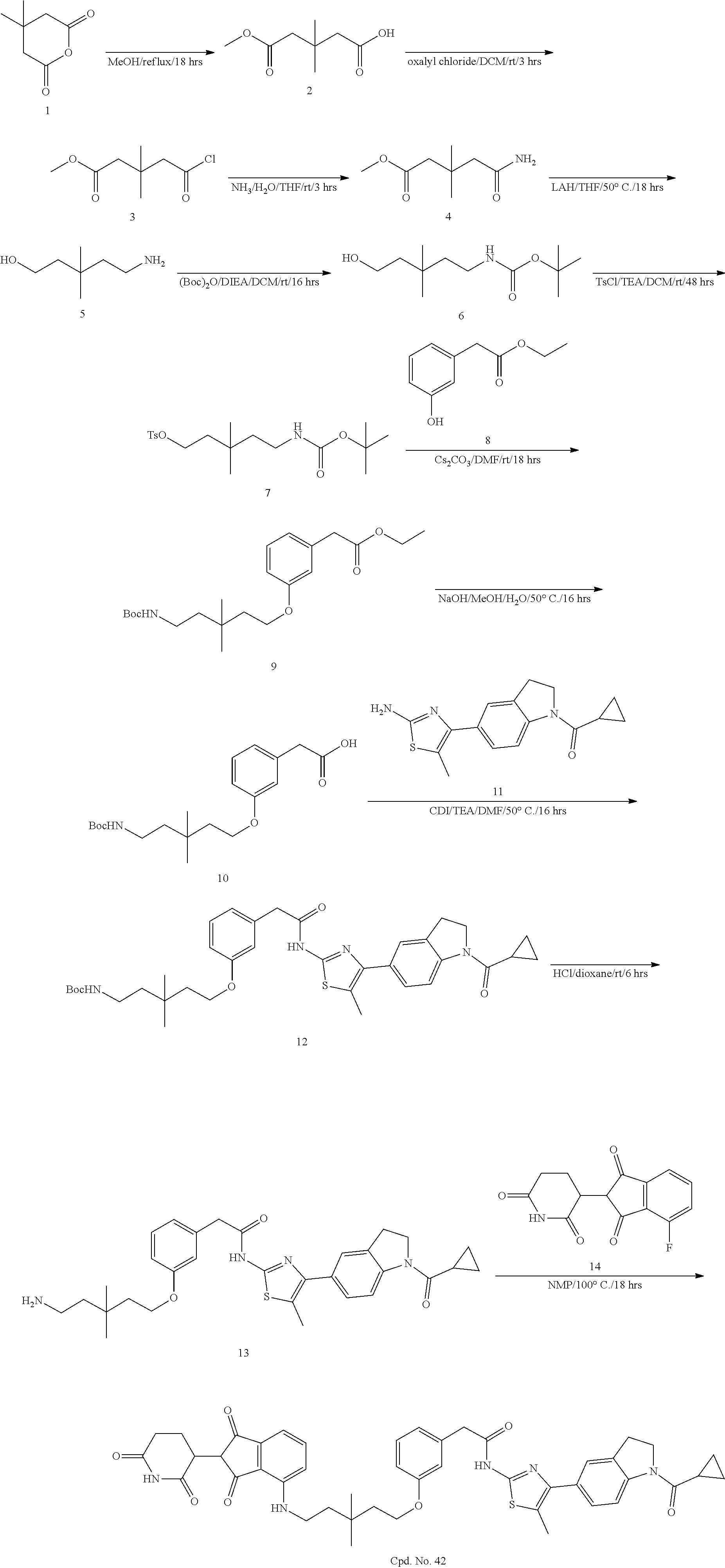

### Step 1. 5-methoxy-3,3-dimethyl-5-oxopentanoic acid

A solution of 4,4-dimethyl-dihydro-3H-pyran-2,6-dione (10.00 g, 70.35 mmol) in methanol (100 mL) was stirred at 60° C. for 18 hrs. The solvent was removed in vacuo to give 5-methoxy-3,3-dimethyl-5-oxopentanoic acid (10.00 g, 816% yield) as a colorless oil. MS (ESI) m/z: 175.1 [M+H]⁺.

### Step 2. methyl 5-chloro-3,3-dimethyl-5-oxopentanoate

To a solution of 5-methoxy-3,3-dimethyl-5-oxopentanoic acid (10.00 g, 57.41 mmol) and AN-dimethylformamide (0.1 mL) in dichloromethane (100 mL) was added oxalyl chloride (7.65 g, 60.28 mmol) at 0° C. After addition, the mixture was stirred at room temperature for 3 hrs. The solvent was removed in vacuo to give methyl 5-chloro-3,3-dimethyl-5-oxopentanoate (12.40 g, overweight) as a yellow oil.

### Step 3. methyl 5-amino-3,3-dimethyl-5-oxopentanoate

To a solution of ammonium hydroxide (50 mL) in tetrahydrofuran (50 mL) was added methyl 5-chloro-3,3-dimethyl-5-oxopentanoate (12.40 g, 57.41 mmol theoretical maximum) at 0° C. After addition, the mixture was stirred at room temperature for 3 hrs. The mixture was diluted with water (50 mL), extracted with dichloromethane (80 mL×4), dried over anhydrous sodium sulfate, filtered and concentrated in vacuo to give methyl 5-amino-3,3-dimethyl-5-oxopentanoate (15.00 g, overweight) as a yellow oil. MS (ESI) m/z: 174.1 [M+H]⁺.

### Step 4. 5-amino-3,3-dimethylpentan-1-ol

To a solution of methyl 5-amino-3,3-dimethyl-5-oxopentanoate (14.90 g, 57.0 mmol theoretical maximum) in tetrahydrofuran (100 mL), was added lithium aluminum hydride (3.50 g, 92.34 mmol) at 0° C. After addition, the mixture was stirred at 50° C. for 18 hrs. The mixture was cooled to room temperature, quenched with water, filtered. The filter cake was washed with dichloromethane (50 mL×3), then the combined filtrate was dried over anhydrous sodium, filtered and concentrated to give 5-amino-3,3-dimethylpentan-1-ol (10.00 g, overweight) as yellow oil. MS (ESI) m/z: 133.2 [M+H]⁺.

### Step 5. tert-butyl 5-hydroxy-3,3-dimethylpentylcarbamate

A solution of 5-amino-3,3-dimethylpentan-1-ol (10.00 g, 57.0 mmol theoretical maximum), di-tert-butyl dicarbonate (19.96 g, 91.45 mmol) and triethylamine (19.28 g, 190.5 mmol) in dichloromethane (100 mL) was stirred at room temperature for 16 hrs. The mixture was diluted with water (100 mL), extracted with dichloromethane (100 mL×3), washed with brine (80 mL), dried over anhydrous sodium sulfate, filtered and concentrated in vacuo. The residue was purified by flash column chromatography (ethyl acetate/petroleum ether 1/3) to give tert-butyl 5-hydroxy-3,3-dimethylpentylcarbamate (10.00 g, 75.8% overall yield) as a yellow oil. MS (ESI) m/z: 232.1 [M+H]⁺.

### Step 6. 5-((tert-butoxycarbonyl)amino)-3,3-dimethylpentyl 4-methylbenzenesulfonate

A solution tert-butyl 5-hydroxy-3,3-dimethylpentylcarbamate (10.00 g, 43.23 mmol), tosyl chloride (12.36 g, 64.84 mmol) and triethylamine (13.12 g, 129.7 mmol) in dichloromethane (100 mL) was stirred at room temperature for 48 hrs. The mixture was diluted with water (100 mL), extracted with dichloromethane (100 mL×3), washed with brine (80 mL), dried over anhydrous sodium sulfate, filtered and concentrated in vacuo. The residue was purified by flash column chromatography (ethyl acetate/petroleum ether=1/4) to give 5-((tert-butoxycarbonyl)amino)-3,3-dimethylpentyl 4-methylbenzenesulfonate (3.00 g, 18.0% yield) as a yellow solid. MS (ESI) m/z: 285.8 [M+H−100]⁺.

### Step 7. ethyl 2-(3-((5-((tert-butoxycarbonyl)amino)-3,3-dimethylpentyl)oxy)phenyl)acetate

A solution of 5-((tert-butoxycarbonyl)amino)-3,3-dimethylpentyl-4-methylbenzenesulfonate (2.98 g, 7.73 mmol), ethyl 2-(3-hydroxyphenyl)acetate (1.39 g, 7.73 mmol) and cesium carbonate (7.56 g, 23.19 mmol) in N,N-dimethylformamide (25 mL) was stirred at room temperature for 18 hrs. The mixture was diluted with water (50 mL), extracted with dichloromethane (50 mL×3), washed with brine (40 mL), dried over anhydrous sodium sulfate, filtered and concentrated in vacuo. The residue was purified by flash column chromatography (ethyl acetate/petroleum ether=1/3) to give ethyl 2-(3-((5-((tert-butoxycarbonyl)amino)-3,3-dimethylpentyl)oxy)phenyl)acetate (1.60 g, 52.6% yield) as yellow solid. MS (ESI) m/z: 294.1 [M+H−100]⁺.

### Step 8. 2-(3-((5-((tert-butoxycarbonyl)amino)-3,3-dimethylpentyl)oxy)phenyl)acetic acid

A solution of ethyl 2-(3-(5-(tert-butoxycarbonyl)-3,3-dimethylpentyloxy)phenyl)acetate (1.58 g, 4.02 mmol) and sodium hydroxide (0.64 g, 16.1 mmol) in methanol (20 mL) and water (20 mL) was stirred at 50° C. for 16 hrs. The mixture was cooled to room temperature, acidified with aqueous hydrochloric acid solution (3 M) to pH=4, extracted with dichloromethane (40 mL×3), washed with brine (30 mL), dried over anhydrous sodium sulfate, filtered and concentrated in vacuo to give 2-(3-((5-((tert-butoxycarbonyl)amino)-3,3-dimethylpentyl)oxy)phenyl)acetic acid (0.70 g, 48% yield) as a white solid. MS (ESI) m/z: 266.1 [M+H−100]⁺.

### Step 9. tert-butyl 5-(3-(2-(4-(1-(cyclopropanecarbonyl)indolin-5-yl)-5-methylthiazol-2-ylamino)-2-oxoethyl)phenoxy)-3,3-dimethylpentylcarbamate

A solution of 2-(3-((5-((tert-butoxycarbonyl)amino)-3,3-dimethylpentyl)oxy)phenyl)acetic acid (0.27 g, 0.74 mmol), triethylamine (0.30 g, 3.0 mmol) and 1,1′-carbonyldiimidazole (0. 18 g, 1.1 mmol) in N,N-dimethylformamide (15 mL) was stirred at room temperature for 1 hr. (5-(2-Amino-5-methylthiazol-4-yl)indolin-1-yl) (cyclopropyl)methanone (0.27 g, 0.89 mmol) was added into the resulting mixture. The mixture was stirred at 50° C. for 16 hours. The mixture was diluted with water (30 mL), extracted with dichloromethane (20 mL×3), dried over anhydrous sodium sulfate, filtered and concentrated in vacuo. The residue was purified by flash column chromatography (methanol/dichloromethane=1/10) to give tert-butyl 5-(3-(2-(4-(1-(cyclopropanecarbonyl)indolin-5-yl)-5-methylthiazol-2-ylamino)-2-oxoethyl)phenoxy)-3,3-dimethylpentylcarbamate (0.24 g, 50% yield) as a white solid. MS (ESI) m/z: 647.1 [M+H]⁺.

### Step 10. 2-(3-(5-amino-3,3-dimethylpentyloxy)phenyl)-N-(4-(1-(cyclopropanecarbonyl)indolin-5-yl)-5-methylthiazol-2-yl)acetamide

A solution of tert-butyl 5-(3-(2-(4-(1-(cyclopropanecarbonyl)indolin-5-yl)-5-methylthiazol-2-ylamino)-2-oxoethyl)phenoxy)-3,3-dimethylpentylcarbamate (0.23 g, 0.36 mmol) in hydrochloric acid (2 M in 1,4-dioxane, 15 mL) was stirred at room temperature for 6 hrs. The solvent was removed in vacuo and the residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15 min) to give 2-(3-(5-amino-3,3-dimethylpentyloxy)phenyl)-N-(4-(1-(cyclopropanecarbonyl)indolin-5-yl)-5-methylthiazol-2-yl)acetamide (0.11 g, 56% yield) as a white solid. IS (ESI) m/z: 547.2 [M+H]⁺.

### Step 11. V-(4-(1-(cyclopropanecarbonyl)indolin-5-yl)-5-methylthiazol-2-yl)-2-(3-(5-(2-(2,6-dioxopiperidin-3-yl)-1,3-dioxoisoindolin-4-ylamino)-3,3-dimethylpentyloxy)phenyl)acetamide

A solution of 2-(3-(5-amino-3,3-dimethylpentyloxy)phenyl)-N-(4-(1-(cyclopropanecarbonyl)indolin-5-yl)-5-methylthiazol-2-yl)acetamide (0.10 g, 0.18 mmol), 2-(2,6-dioxopiperidin-3-yl)-4-fluoroisoindoline-1,3-dione (0.25 g, 0.91 mmol) and N,N-diisopropylethylamine (0.12 g, 0.91 mmol) in 1-methylpyrrolidin-2-one (8 mL) was stirred at 100° C. for 18 hrs. The mixture was diluted with water (20 mL), extracted with dichloromethane (35 mL×3), washed with brine (30 mL), dried over anhydrous sodium sulfate, filtered and concentrated in vacuo. The residue was purified by prep-HPLC (Column: Welch Xtimate 21.2*250 mm C18, 10 Um, Mobile Phase: A: water (10 mmol/L ammonium bicarbonate) B: acetonitrile; B %: 30%-70% in 15 min) to give N-(4-(1-(cyclopropanecarbonyl)indolin-5-yl)-5-methylthiazol-2-yl)-2-(3-(5-(2-(2,6-dioxopiperidin-3-yl)-1,3-dioxoisoindolin-4-ylamino)-3,3-dimethylpentyloxy)phenyl)acetamide (38.2 mg, 26% yield) as a yellow solid. ¹H NMR (500 MHz, DMSO-d₆): δ 12.28 (s, 1H), 11.09 (s, 1H), 8.06 (d, J=7.0 Hz, 1H), 7.55 (dd, J=8.0 Hz, 7.0 Hz, 1H), 7.49 (s, 1H), 7.41 (d, J=8.0 Hz, 1H), 7.22 (t, J=8.0 Hz, 1H), 7.06 (d, J=8.5 Hz, 1H), 6.99 (d, J=6.5 Hz, 1H), 6.91 (s, 1H), 6.87 (d, J=8.0 Hz, 1H), 6.83 (dd, J=8.5 Hz, 2.0 Hz, 1H), 6.50 (t, J=5.4 Hz, 1H), 5.04 (dd, J=13.0 Hz, 5.5 Hz, 1H), 4.33 (t, J=7.0 Hz, 2H), 4.04 (t, J=7.0 Hz, 2H), 3.69 (s, 2H), 3.36-3.32 (m, 2H), 3.22 (t, J=7.5 Hz, 2H), 2.89-2.83 (m, 1H), 2.58-2.51 (m, 2H), 2.35 (s, 3H), 2.02-1.96 (m, 2H), 1.72 (t, J=7.5 Hz, 2H), 1.58 (t, J=8.5 Hz, 2H), 1.02 (s, 6H), 0.87-0.84 (m, 4H). MS (ESI) m/z: 802.6 [M+H]⁺.

### Example 44

### Synthesis of N-(4-(1-(cyclopropanecarbonyl)indolin-5-yl)-5-methylthiazol-2-yl)-2-(3-(2-((2-(2-(2,6-dioxopiperidin-3-yl)-1,3-dioxoisoindolin-4-ylamino)ethyl)(methyl)amino)ethoxy)phenyl)acetamide (Cpd. No. 44)

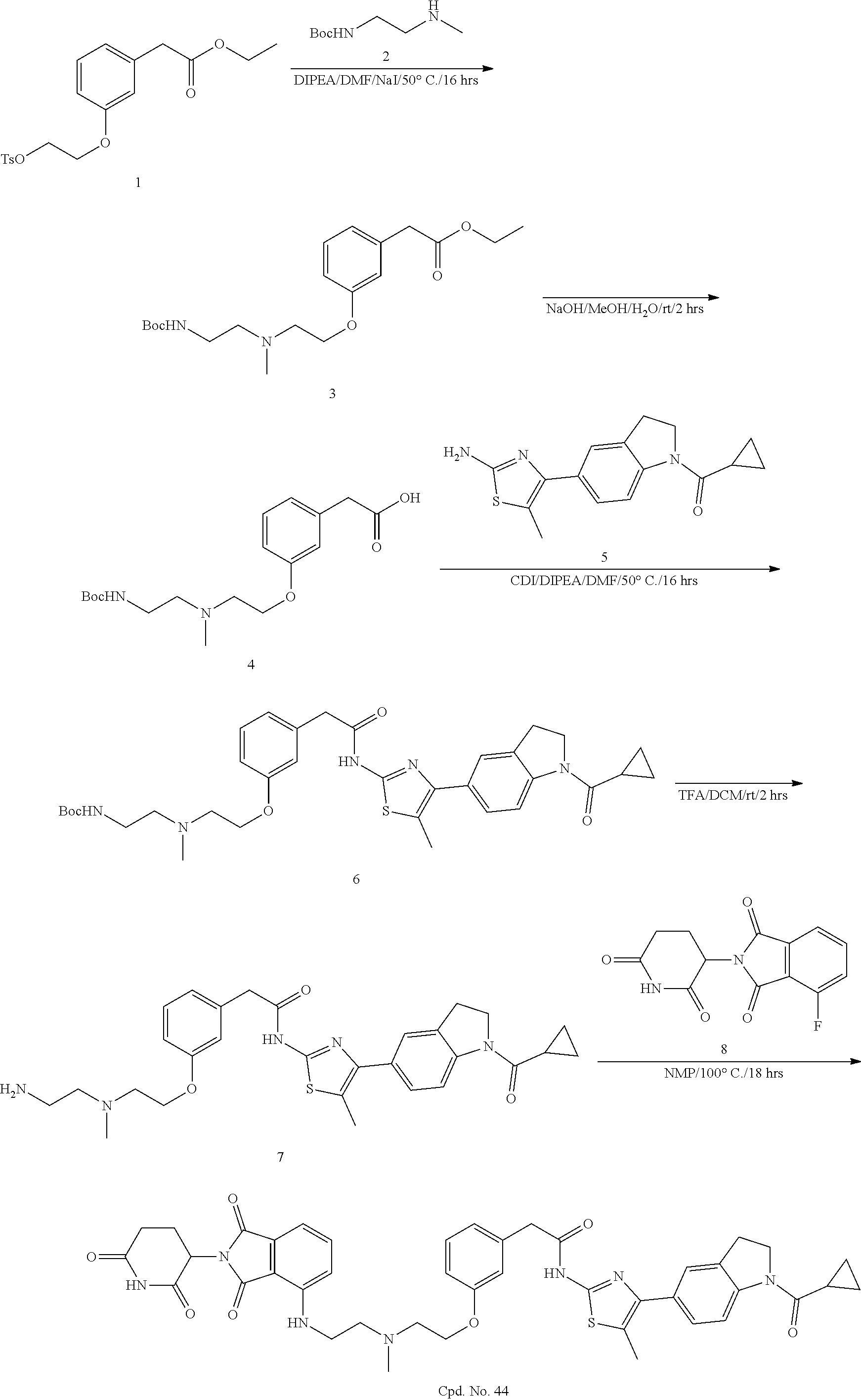

### Step 1. ethyl 2-(3-(2-((2-((tert-butoxycarbonyl)amino)ethyl)(methyl)amino)ethoxy)phenyl)acetate

A solution of ethyl 2-(3-(2-(tosyloxy)ethoxy)phenyl)acetate (1.00 g, 2.64 mmol), tert-butyl 2-(methylamino)ethylcarbamate (0.92 g, 5.3 mmol), N,N-diisoproylethylamine (1.37 g, 10.6 mmol) and sodium iodide (0.04 g, 0.26 mmol) in N,N-dimethylfornamide (30 mL) was stirred at 50° C. for 16 hrs. The mixture was diluted with water (50 mL), extracted with dichloromethane (80 mL×2), washed with brine (40 mL×3), dried over anhydrous sodium sulfate, filtered and concentrated in vacuo. The residue was purified by flash column chromatography (ethyl acetate/petroleum ether=2/3) to give ethyl 2-(3-(2-((2-((tert-butoxycarbonyl)amino)ethyl)(methyl)amino)ethoxy)phenyl)acetate (1.30 g, overweight) as a yellow solid. MS (ESI) mi/z: 381.5 [M+H]⁺.

### Step 2. 2-(3-(2-((2-((tert-butoxycarbonyl)amino)ethyl)(methyl)amino)ethoxy)phenyl)acetic acid

A solution of ethyl 2-(3-(2-((2-((tert-butoxycarbonyl)amino)ethyl)(methyl)amino)ethoxy)phenyl)acetate (1.30 g, 2.64 mmol theoretical maximum) and sodium hydroxide (0.55 g, 13.7 mmol) in methanol (20 mL) and water (20 mL) was stirred at room temperature for 2 hrs. The mixture was cooled to room temperature, acidified with aqueous hydrochloric acid solution (3 M) to pH=4, extracted with dichloromethane (40 mL×3), washed with brine (30 mL), dried over anhydrous sodium sulfate, filtered and concentrated in vacuo to give 2-(3-(2-((2-((tert-butoxycarbonyl)amino)ethyl)(methyl)amino)ethoxy)phenyl)acetic acid (1.02 g, overweight) as a white solid. MS (ESI) m/z: 353.4 [M+H]⁺.

### Step 3. tert-butyl 2-((2-(3-(2-(4-(1-(cyclopropanecarbonyl)indolin-5-yl)-5-methylthiazol-2-ylamino)-2-oxoethyl)phenoxy)ethyl)(methyl)amino)ethylcarbamate

A solution of 2-(3-(2-((2-((tert-butoxycarbonyl)amino)ethyl)(methyl)amino) ethoxy)phenyl)acetic acid (1.00 g, 2.59 mmol theoretical maximum), N,N-diisoproylethylamine (1.10 g, 8.51 mmol) and 1,1′-carbonyldiimidazole (0.69 g, 4.3 mmol) in N,N-dimethylformamide (35 mL) was stirred at room temperature for 1 hr. (5-(2-Amino-5-methylthiazol-4-yl) indolin-1-yl)(cyclopropyl)methanone (0.85 g, 2.84 mmol) was added into the resulting mixture. The mixture was stirred at 50° C. for 16 hours. The mixture was diluted with water (60 mL), extracted with dichloromethane (50 mL×3), dried over anhydrous sodium sulfate, filtered and concentrated in vacuo. The residue was purified by flash column chromatography (methanol/dichloromethane=1/10) to give tert-butyl 2-((2-(3-(2-(4-(1-(cyclopropanecarbonyl)indolin-5-yl)-5-methylthiazol-2-ylamino)-2-oxoethyl)phenoxy)ethyl)(methyl)amino)ethylcarbamate (1.30 g, 79.2% overall yield) as a white solid. MS (ESI) m/z: 634.3 [M+H]⁺.

### Step 4. 2-(3-(2-((2-aminoethyl)(methyl)amino)ethoxy)phenyl)-N-(4-(1-(cyclopropanecarbonyl) indolin-5-yl)-5-methylthiazol-2-yl)acetamide

A solution of tert-butyl 2-((2-(3-(2-(4-(1-(cyclopropanecarbonyl)indolin-5-yl)-5-methylthiazol-2-ylamino)-2-oxoethyl)phenoxy)ethyl)(methyl)amino)ethylcarbamate (1.20 g, 1.89 mmol) in trifluoroacetic acid (30 mL) and dichloromethane (30 mL) was stirred at room temperature for 2 hrs. The solvent was removed in vacuo and the residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15 min) to give 2-(3-(2-((2-aminoethyl)(methyl)amino)ethoxy)phenyl)-N-(4-(1-(cyclopropanecarbonyl)indolin-5-yl)-5-methylthiazol-2-yl)acetamide (0.11 g, 11% yield) as a white solid. MS (ESI) m/z: 534.2 [M+H]⁺.

### Step 5. N-(4-(1-(cyclopropanecarbonyl)indolin-5-yl)-5-methylthiazol-2-yl)-2-(3-(2-((2-(2-(2,6-dioxopiperidin-3-yl)-1,3-dioxoisoindolin-4-ylamino)ethyl)(methyl)amino)ethoxy)phenyl)acetamide

A solution of 2-(3-(2-((2-aminoethyl)(methyl)amino)ethoxy)phenyl)-N-(4-(1-(cyclopropanecarbonyl)indolin-5-yl)-5-methylthiazol-2-yl)acetamide (98.0 mg, 0.18 mmol), 2-(2,6-dioxopiperidin-3-yl)-4-fluoroisoindoline-1,3-dione (0.25 g, 0.92 mmol) and N,N-diisopropylethylamine (0.12 g, 0.92 mmol) in 1-methylpyrrolidin-2-one (8 mL) was stirred at 100° C. for 18 hrs. The mixture was diluted with water (20 mL), extracted with dichloromethane (40 mL×3), washed with brine (30 mL), dried over anhydrous sodium sulfate, filtered and concentrated in vacuo. The residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15 min) to give N-(4-(1-(cyclopropanecarbonyl)indolin-5-yl)-5-methylthiazol-2-yl)-2-(3-(2-((2-(2-(2,6-dioxopiperidin-3-yl)-1,3-dioxoisoindolin-4-ylamino)ethyl)(methyl)amino)ethoxy) phenyl)acetamide (8.0 mg, 5.5% yield) as a yellow solid. ¹H NMR (400 MHz, DMSO-d₆): δ 12.28 (s, 1H), 11.09 (s, 1H), 8.05 (d, J=7.2 Hz, 1H), 7.56 (t, J=8.0 Hz, 1H), 7.49 (s, 1H), 7.41 (d, J=8.4 Hz, 1H), 7.19 (t, J=7.6 Hz, 1H), 7.07 (d, J=8.4 Hz, 1H), 7.00 (d, J=7.2 Hz, 1H), 6.89 (s, 1H), 6.86 (d, J=7.2 Hz, 1H), 6.80 (d, J=8.4 Hz, 1H), 6.74 (t, J=4.8 Hz, 1H), 5.04 (dd, J=13.0 Hz, 5.5 Hz, 1H), 4.33 (t, J=7.0 Hz, 2H), 4.04 (t, J=7.0 Hz, 2H), 3.66 (s, 2H), 3.36-3.39 (m, 2H), 3.24 (t, J=8.0 Hz, 2H), 2.90-2.82 (m, 3H), 2.72-2.68 (m, 2H), 2.58-2.51 (m, 2H), 2.42 (s, 3H), 2.33 (s, 3H), 2.02-1.96 (m, 2H), 0.87-0.84 (m, 4H). MS (ESI) m/z: 789.6 [M+H]⁺.

### Example 45

### Synthesis of N-(4-(1-(cyclopropanecarbonyl)indolin-5-yl)-5-methylthiazol-2-yl)-2-(3-(5-(2-(2,6-dioxopiperidin-3-yl)-1,3-dioxoisoindolin-4-yloxy)pentyloxy)phenyl)acetamide (Cpd. No. 46)

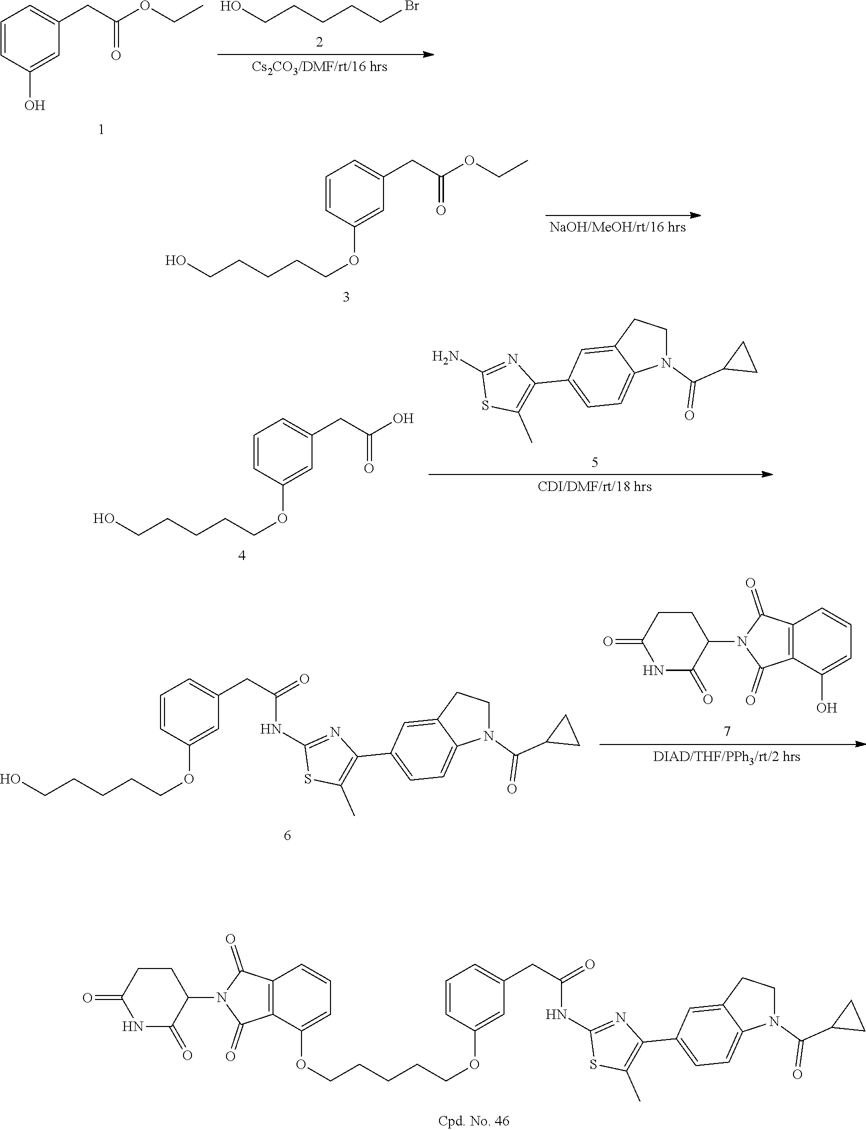

### Step 1. ethyl 2-(3-((5-hydroxypentyl)oxy)phenyl)acetate

A solution of ethyl 2-(3-hydroxyphenyl)acetate (2.00 g, 11.1 mmol), 5-bromopentan-1-ol (1.86 g, 11.1 mmol) and cesium carbonate (5.43 g, 16.7 mmol) in N,N-dimethylformamide (30 mL) was stirred at room temperature for 16 hrs. The reaction was quenched with water (50 mL), extracted with dichloromethane (80 mL×3), washed with brine (30 mL×2), dried over anhydrous sodium sulfate, filtered and concentrated in vacuo. The residue was purified by flash column chromatography (ethyl acetate/petroleum ether=1/4) to give ethyl 2-(3-((5-hydroxypentyl)oxy)phenyl)acetate (3.00 g, overweight) as white solid. MS (ESI) m/z: 267.0 [M+H]⁺.

### Step 2. 2-(3-((5-hydroxypentyl)oxy)phenyl)acetic acid

A solution of ethyl 2-(3-((5-hydroxypentyl)oxy)phenyl)acetate (3.00 g, 11.3 mmol theoretical maximum) and sodium hydroxide (1.12 g, 28.2 mmol) in methanol (25 mL) and water (15 mL) was stirred at room temperature for 16 hrs. The mixture was quenched with water (20 mL), acidified to pH=3-4 with 2 N hydrochloric acid aqueous solution, extracted with dichloromethane (50 mL×2), washed with brine (30 mL), dried over anhydrous sodium sulfate, filtered and concentrated in vacuo to give 2-(3-((5-hydroxypentyl)oxy)phenyl)acetic acid (2.50 g, 93.1% overall yield for two steps) as white solid. MS (ESI) m/z: 239.2 [M+H]⁺.

### Step 3. N-(4-(1-(cyclopropanecarbonyl)indolin-5-yl)-5-methylthiazol-2-yl)-2-(3-((5-hydroxypentyl)oxy)phenyl)acetamide

A solution of 2-(3-((5-hydroxypentyl)oxy)phenyl)acetic acid (2.30 g, 9.65 mmol), 1,1′-carbonyldiimidazole (2.35 g, 14.5 mmol) and N,N-diisopropylethylamine (4.99 g, 38.6 mmol) in N,N-dimethylformamide (25 mL) was stirred at room temperature for 30 mins. (5-(2-amino-5-methylthiazol-4-yl)indolin-1-yl)(cyclopropyl)methanone (2.89 g, 9.65 mmol) was added into the resulting mixture. The reaction was stirred at room temperature for 18 hrs. The reaction mixture was diluted with water (40 ml), extracted with dichloromethane (80 mL×3), dried over anhydrous sodium sulfate, filtered and concentrated in vacuo. The residue was purified by Pre-HPLC (Column: Welch Xtimate 21.2*250 mm C18, 10 m, Mobile Phase: A: water (10 mmol/L ammonium bicarbonate) B: acetonitrile; B %: 30%-70% in 15 min) to give N-(4-(1-(cyclopropanecarbonyl)indolin-5-yl)-5-methylthiazol-2-yl)-2-(3-((5-hydroxypentyl)oxy)phenyl)acetamide (0.18 g, 3.6% yield) as white solid. MS (ESI) m/z: 520.3 [M+H]⁺.

### Step 4. N-(4-(1-(cyclopropanecarbonyl)indolin-5-yl)-5-methylthiazol-2-yl)-2-(3-((5-((2-(2,6-dioxopiperidin-3-yl)-1,3-dioxoisoindolin-4-yl)oxy)pentyl)oxy)phenyl)acetamide

To a solution of N-(4-(1-(cyclopropanecarbonyl)indolin-5-yl)-5-methylthiazol-2-yl)-2-(3-((5-hydroxypentyl)oxy)phenyl)acetamide (90.0 mg, 0.173 mmol), 2-(2,6-dioxopiperidin-3-yl)-4-hydroxyisoindoline-1,3-dione (47.8 mg, 0.174 mmol) and triphenylphosphine (68.2 mg, 0.26 mmol) in tetrahydrofuran (10 mL), was added diisopropyl azodiformate (52.6 mg, 0.26 mmol) at room temperature. After addition, the mixture was stirred at room temperature for 2 hrs. The mixture was diluted with water (30 mL), extracted with dichloromethane (40 mL×3), dried over anhydrous sodium sulfate, filtered and concentrated in vacuo. The residue was purified by Pre-HPLC (Column: Welch Xtimate 21.2*250 mm C18, 10 μm, Mobile Phase: A: water (10 mmol/L ammonium bicarbonate) B: acetonitrile; B %: 30%-70% in 15 min) to give N-(4-(1-(cyclopropanecarbonyl)indolin-5-yl)-5-methylthiazol-2-yl)-2-(3-(5-(2-(2,6-dioxopiperidin-3-yl)-1,3-dioxoisoindolin-4-yloxy)pentyloxy)phenyl)acetamide (72.8 mg, 54.2% yield) as white solid. ¹H NMR (400 MHz, DMSO-d₆): δ 12.30 (s, 1H), 11.11 (s, 1H), 8.06 (d, J=7.6 Hz, 1H), 7.80 (t. J=8.0 Hz, 1H), 7.52-7.49 (m, 2H), 7.43 (t. J=7.2 Hz, 2H), 7.22 (t, J=8.0 Hz, 1H), 6.91-6.82 (m, 3H), 5.08 (dd, J=13.0, 5.4 Hz, 1H), 4.33 (t, J=8.4 Hz, 2H), 4.23 (t, J=6.0 Hz, 2H), 3.98 (t, J=6.4 Hz, 2H), 3.69 (s, 2H), 3.22 (t, J=8.4 Hz, 2H), 2.92-2.82 (m, 1H), 2.59-2.50 (m, 2H), 2.45 (s, 3H), 2.07-1.96 (m, 2H), 1.86-1.76 (m, 1.65-1.57 (m, 2H), 0.87-0.85 (m, 4H). MS (ESI) m/z: 776.2 [M+H]⁺.

### Example 46

### Synthesis of N-(4-(1-(cyclopropanecarbonyl)indolin-5-yl)-5-methylthiazol-2-yl)-2-(3-(5-(2-(2,6-dioxopiperidin-3-yl)-1,3-dioxoisoindolin-4-yloxy)-3-methylpentyloxy)phenyl)acetamide (Cpd. No. 47)

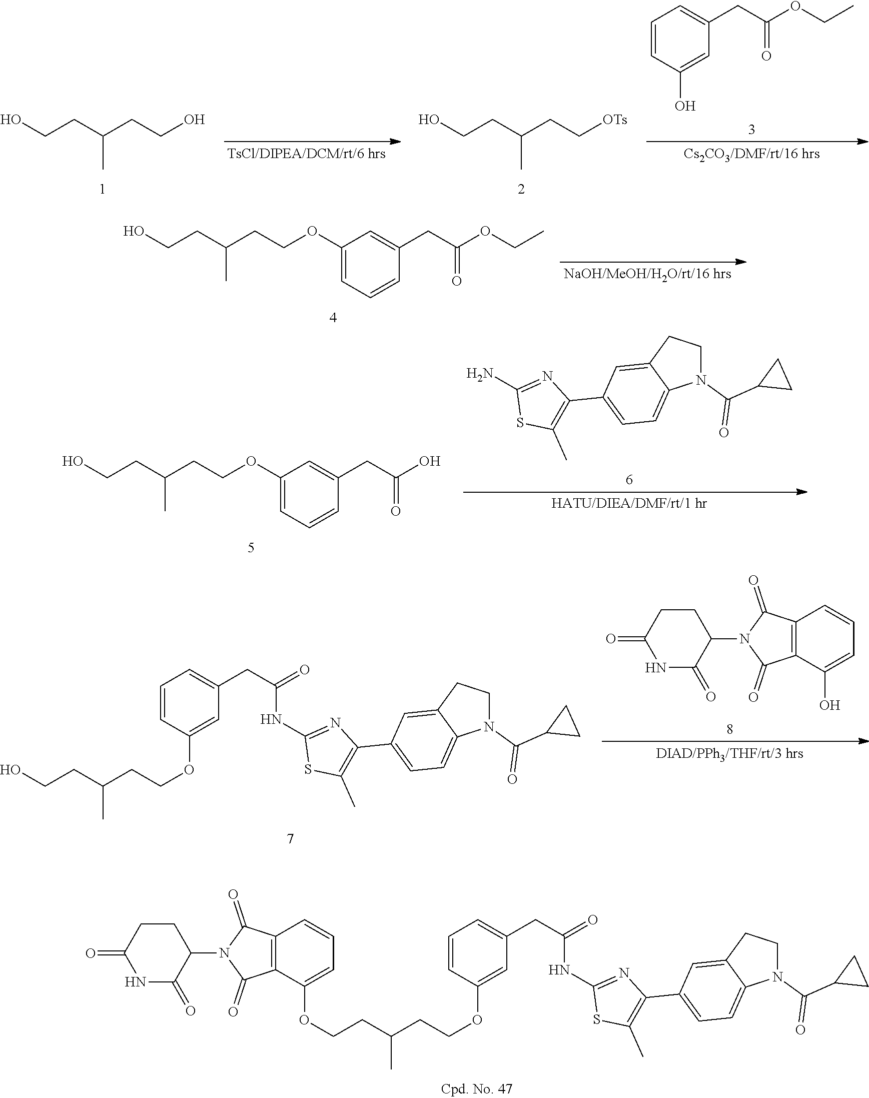

### Step 1. 5-hydroxy-3-methylpentyl 4-methylbenzenesulfonate

A solution of 3-methylpentane-1,5-diol (5.00 g, 42.1 mmol), tosyl chloride (8.07 g, 42.3 mmol) and N,N-diisopropylethylamine (16.41 g, 126.9 mmol) in dichloromethane (100 mL) was stirred at room temperature for 16 hrs. The reaction was quenched with water (100 mL), extracted with dichloromethane (80 mL×3), washed with brine (50 mL), dried over anhydrous sodium sulfate, filtered and concentrated in vacuo. The residue was purified by flash column chromatography (dichloromethane/methanol=20/1) to give 5-hydroxy-3-methylpentyl 4-methylbenzenesulfonate (2.50 g, 21.8% yield) as yellow oil. NIS (ESI) m/z: 273.2 [M+H]⁺.

### Step 2. ethyl 2-(3-((5-hydroxy-3-methylpentyl)oxy)phenyl)acetate

A mixture of 5-hydroxy-3-methylpentyl 4-methylbenzenesulfonate (2.40 g, 8.81 mmol), ethyl 2-(3-hydroxyphenyl)acetate (1.59 g, 8.82 mmol) and cesium carbonate (8.61 g, 26.4 mmol) in N,N-dimethylformamide (20 mL) was stirred at room temperature for 16 hrs. The reaction was quenched with water (80 mL), extracted with dichloromethane (50 mL×2), washed with brine (50 mL), dried over anhydrous sodium sulfate, filtered and concentrated in vacuo. The residue was purified by flash column chromatography (dichloromethane) to give ethyl 2-(3-((5-hydroxy-3-methylpentyl)oxy)phenyl)acetate (1.10 g, 44.5% yield) as yellow solid. MS (ESI) m/z: 281.4 [M+H]⁺.

### Step 3. 2-(3-((5-hydroxy-3-methylpentyl)oxy)phenyl)acetic acid

A solution of ethyl 2-(3-((5-hydroxy-3-methylpentyl)oxy)phenyl)acetate (1.00 g, 3.57 mmol) and sodium hydroxide (0.43 g, 10.7 mmol) in methanol (10 mL) and water (10 mL) was stirred at room temperature for 16 hours. The mixture was diluted with water (20 mL), acidified to pH=3-4 with 3 M hydrochloric acid aqueous solution, extracted with dichloromethane (70 mL×3), washed with brine (30 mL), dried over anhydrous sodium sulfate, filtered and concentrated in vacuo to give 2-(3-((5-hydroxy-3-methylpentyl)oxy)phenyl)acetic acid (0.60 g, 66.7% yield) as a white solid. MS (ESI) m/z: 253.2 [M+H]⁺.

### Step 4. N-(4-(1-(cyclopropanecarbonyl)indolin-5-yl)-5-methylthiazol-2-yl)-2-(3-((5-hydroxy-3-methylpentyl)oxy)phenyl)acetamide

A solution of 2-(3-((5-hydroxy-3-methylpentyl)oxy)phenyl)acetic acid (0.50 g, 1.98 mmol), (5-(2-amino-5-methylthiazol-4-yl)indolin-1-yl)(cyclopropyl)methanone (0.59 g, 1.97 mmol), N,N-diisopropylethylamine (0.77 g, 5.95 mmol) and 2-(7-azabenzotriazol-1-yl)-N,N,N′,N′-tetramethyluronium hexafluorophosphate (1.13 g, 2.97 mmol) in N,N-dimethylformamide (15 mL) was stirred at room temperature for 1 hr. The reaction was diluted with water (30 mL), extracted with dichloromethane (20 mL×3), dried over anhydrous sodium sulfate, filtered and concentrated in vacuo. The residue was purified by flash column chromatography (dichloromethane/methanol=10/1) to give N-(4-(1-(cyclopropanecarbonyl)indolin-5-yl)-5-methylthiazol-2-yl)-2-(3-((5-hydroxy-3-methylpentyl)oxy)phenyl)acetamide (0.13 g, 12% yield) as a yellow solid. MS (ESI) m/z: 533.8 [M+H]⁺.

### Step 5. N-(4-(1-(cyclopropanecarbonyl)indolin-5-yl)-5-methylthiazol-2-yl)-2-(3-((5-((2-(2,6-dioxopiperidin-3-yl)-1,3-dioxoisoindolin-4-yl)oxy)-3-methylpentyl)oxy)phenyl)acetamide

To a solution of N-(4-(1-(cyclopropanecarbonyl)indolin-5-yl)-5-methylthiazol-2-yl)-2-(3-(5-hydroxy-3-methylpentyl)oxy)phenyl)acetamide (50.0 mg, 0.094 mmol), triphenylphosphane (36.9 mg, 0.14 mmol) and 2-(2,6-dioxopiperidin-3-yl)-4-hydroxyisoindoline-1,3-dione (25.7 mg, 0.094 mmol) in tetrahydrofuran (3 mL), was added diisopropyl azodicarboxylate (28.4 mg, 0.14 mmol) at room temperature. After addition, the mixture was stirred at room temperature for 3 hrs. The mixture was diluted with water (10 mL), extracted with dichloromethane (20 mL×3), dried over anhydrous sodium sulfate, filtered and concentrated in vacuo. The residue was purified by Pre-HPLC (Column: Welch Xtimate 21.2*250 mm C18, 10 μm, Mobile Phase: A: water (10 mmol/L ammonium bicarbonate) B: acetonitrile; B %: 30%-70% in 15 min) to give N-(4-(1-(cyclopropanecarbonyl)indolin-5-yl)-5-methylthiazol-2-yl)-2-(3-((5-((2-(2,6-dioxopiperidin-3-yl)-1,3-dioxoisoindolin-4-yl)oxy)-3-methylpentyl)oxy)phenyl) acetamide (4.0 mg, 5.4% yield) as a yellow solid. ¹H NMR (400 MHz, DMSO-d₆): δ 12.30 (s, 1H), 11.10 (s, 1H), 8.06 (d, J=7.6 Hz, 1H), 7.78 (t, J=7.6 Hz, 1H), 7.53-7.49 (m, 2H), 7.43-7.40 (m, 2H), 7.20 (t, J=8.0 Hz, 1H), 6.91 (s, 1H), 6.87 (d, J=7.2 Hz, 1H), 6.82 (d, J=8.4 Hz, 1H), 5.07 (dd, J=12.8 Hz, 5.6 Hz, 1H), 4.32-4.26 (m, 4.04-3.99 (m, 2H), 3.68 (s, 2H), 3.22 (t, J=8.0 Hz, 2H), 2.91-2.82 (m, 1H), 2.59-2.45 (m, 2H), 2.42 (s, 3H), 2.02-1.95 (m, 3H), 1.90-1.80 (m, 2H), 1.70 (m, 2H), 1.03 (d, J=7.6 Hz, 3H), 0.89-0.86 (m, 4H). MS (ESI) m/z: 790.7 [M+H]⁺.

### Example 47

### Synthesis of N-(4-(1-(cyclopropanecarbonyl)indolin-5-yl)-5-methylthiazol-2-yl)-2-(3-((5-((2-(2,6-dioxopiperidin-3-yl)-1,3-dioxoisoindolin-4-yl)oxy)-3,3-dimethylpentyl)oxy)phenyl)acetamide (Cpd. No. 48)

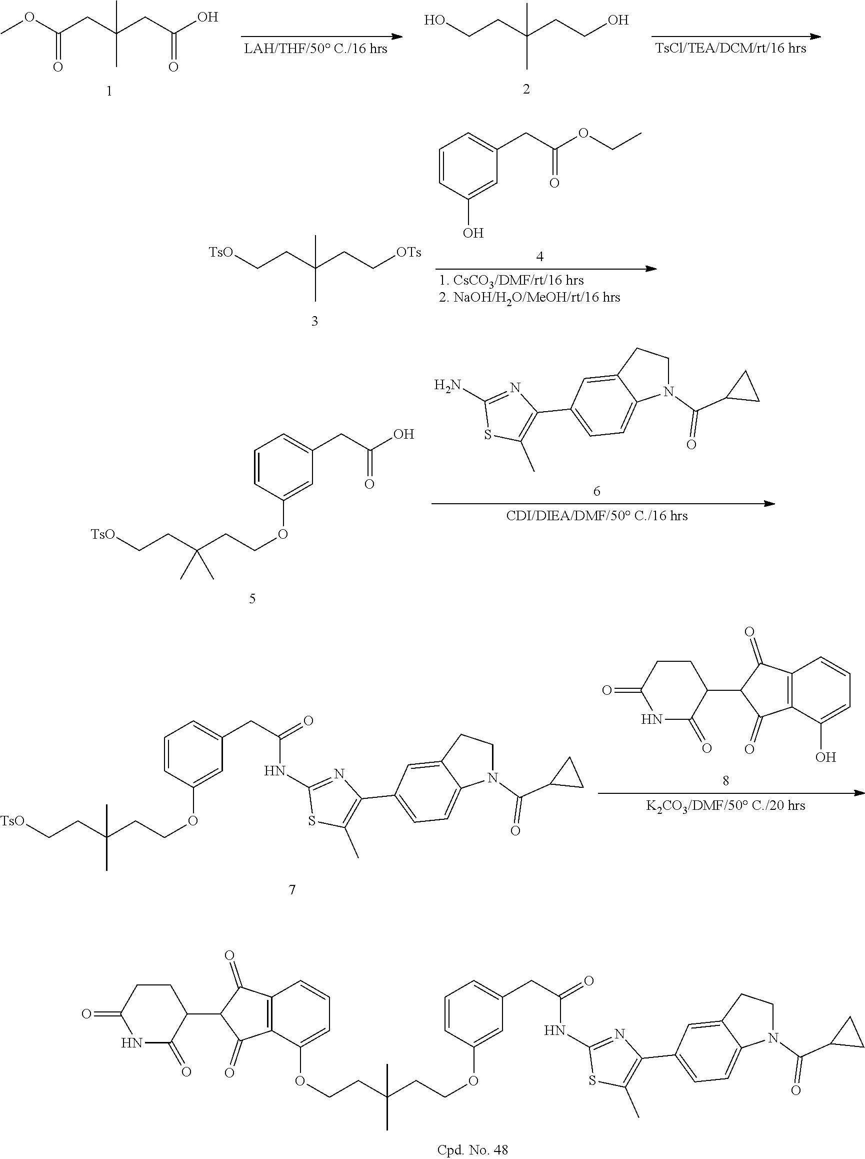

### Step 1. 3,3-dimethylpentane-1,5-diol

To a solution of 5-methoxy-3,3-dimethyl-5-oxopentanoic acid (7.00 g, 40.2 mmol) in tetrahydrofuran (80 mL), was added lithium aluminum hydride (3.05 g, 80.4 mmol) at 0° C. After addition, the mixture was stirred at 50° C. for 16 hrs. The reaction mixture was cooled to room temperature, quenched with water, filtered. The filter cake was washed with dichloromethane (50 mL×3), then the combined filtrate was dried over anhydrous sodium, filtered and concentrated to give 3,3-dimethylpentane-1,5-diol (4.00 g, 75.3% yield) as yellow oil. MS (ESI) m/z: 133.2 [M+H]⁺.

### Step 2. 3,3-dimethylpentane-1,5-diyl bis(4-methylbenzenesulfonate)

A solution of 3,3-dimethylpentane-1,5-diol (3.00 g, 22.7 mmol), tosyl chloride (12.98 g, 68.08 mmol) and triethylamine (9.19 g, 90.8 mmol) in dichloromethane (50 mL) was stirred at room temperature for 16 hrs. The mixture was diluted with water (50 mL), extracted with dichloromethane (50 mL×3), washed with brine (80 mL), dried over anhydrous sodium sulfate, filtered and concentrated in vacuo. The residue was purified by flash column chromatography (ethyl acetate/petroleum ether=1/4) to give 3,3-dimethylpentane-1,5-diyl bis(4-methylbenzenesulfonate) (3.00 g, 30.0% yield) as a white solid. MS (ESI) m/z: 441.2 [M+H]⁺.

### Step 3. 2-(3-((3,3-dimethyl-5-(tosyloxy)pentyl)oxy)phenyl)acetic acid

A solution of 3,3-dimethylpentane-1,5-diyl bis(4-methylbenzenesulfonate) (3.00 g, 6.81 mmol), ethyl 2-(3-hydroxyphenyl)acetate (1.22 g, 6.81 mmol) and cesium carbonate (4.44 g, 13.6 mmol) in N,N-dimethylformamide (30 mL) was stirred at room temperature for 16 hrs. Methanol (20 mL), water (20 mL) and sodium hydroxide (0.82 g, 20.43 mmol) were added into the resulting mixture. The mixture was stirred at room temperature for 16 hrs. The mixture was acidified with aqueous hydrochloric acid solution (3 M) to pH=4, extracted with dichloromethane (50 mL×3), washed with brine (40 mL), dried over anhydrous sodium sulfate, filtered and concentrated in vacuo to give 2-(3-((3,3-dimethyl-5-(tosyloxy)pentyl)oxy)phenyl)acetic acid (0.40 g, 14.0% yield) as a white solid. MS (ESI) m/z: 421.2 [M+H]⁺.

### Step 4. 5-(3-(2-((4-(1-(cyclopropanecarbonyl)indolin-5-yl)-5-methylthiazol-2-yl)amino)-2-oxoethyl)phenoxy)-3,3-dimethylpentyl 4-methylbenzenesulfonate

A solution of 2-(3-((3,3-dimethyl-5-(tosyloxy)pentyl)oxy)phenyl)acetic acid (0.30 g, 0.71 mmol), N,N-diisopropylethylamine (0.18 g, 1.4 mmol) and 1,1′-carbonyldiimidazole (0.17 g, 1.1 mmol) in N,N-dimethylformamide (15 mL) was stirred at room temperature for 30 mins. (5-(2-Amino-5-methylthiazol-4-yl)indolin-1-yl) (cyclopropyl)methanone (0.22 g, 0.75 mmol) was added into the resulting mixture. The mixture was stirred at 50° C. for 16 hours. The mixture was diluted with water (30 mL), extracted with dichloromethane (20 mL×3), dried over anhydrous sodium sulfate, filtered and concentrated in vacuo. The residue was purified by flash column chromatography (methanol/dichloromethane=1/10) to give 5-(3-(2-((4-(1-(cyclopropanecarbonyl)indolin-5-yl)-5-methylthiazol-2-yl)amino)-2-oxoethyl)phenoxy)-3,3-dimethylpentyl 4-methylbenzenesulfonate (0.10 g, 20.0% yield) as a white solid. MS (ESI) m/z: 702.0 [M+H]⁺.

### Step 5. N-(4-(1-(cyclopropanecarbonyl)indolin-5-yl)-5-methylthiazol-2-yl)-2-(3-((5-((2-(2,6-dioxopiperidin-3-yl)-1,3-dioxoisoindolin-4-yl)oxy)-3,3-dimethylpentyl)oxy)phenyl)acetamide

A solution of 5-(3-(2-((4-(1-(cyclopropanecarbonyl)indolin-5-yl)-5-methylthiazol-2-yl)amino)-2-oxoethyl)phenoxy)-3,3-dimethylpentyl-4-methylbenzenesulfonate (90.0 mg, 0.13 mmol), 2-(2,6-dioxopiperidin-3-yl)-4-hydroxyisoindoline-1,3-dione (36.9 mg, 0.13 mmol) and potassium carbonate (53.2 mg, 0.38 mmol) in N,N-dimethylformamide (2 mL) was stirred at 50° C. for 20 hrs. The mixture was diluted with water (10 mL), extracted with dichloromethane (30 mL×2), washed with brine (10 mL), dried over anhydrous sodium sulfate, filtered and concentrated in vacuo. The residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15 min) to give N-(4-(1-(cyclopropanecarbonyl)indolin-5-yl)-5-methylthiazol-2-yl)-2-(3-((5-((2-(2,6-dioxopiperidin-3-yl)-1,3-dioxoisoindolin-4-yl)oxy)-3,3-dimethylpentyl)oxy)phenyl)acetamide (28.3 mg, 27.5% yield) as a white solid. ¹H NMR (400 MHz, DMSO-d₆): δ 12.23 (s, 1H), 11.18 (s, 1H), 8.06 (s, 1H), 7.81 (s, 1H), 7.56-7.55 (m, 1H), 7.51 (s, 1H), 7.47-7.43 (m, 2H), 7.25-7.23 (m, 1H), 6.92-6.90 (m, 2H), 6.85-6.84 (m, 1H), 5.16-5.11 (m, 1H), 4.42 (s, 4H), 4.12 (s, 2H), 3.75 (s, 2H), 3.28 (s, 2H), 2.89-2.83 (m, 1H), 2.58-2.51 (m, 2H), 2.45 (s, 3H), 2.07-2.03 (m, 2H), 1.87-1.84 (m, 1.12 (s, 6H), 0.94 (brs, 4H). MS (ESI) m/z: 804.0 [M+H]⁺.

### Example 48

### Synthesis of N-(4-(1-(cyclopropanecarbonyl)indolin-5-yl)-5-methylthiazol-2-yl)-2-(3-(5-(2-(2,6-dioxopiperidin-3-yl)-1,3-dioxoisoindolin-5-ylamino)pentyloxy)phenyl)acetamide (Cpd. No. 49)

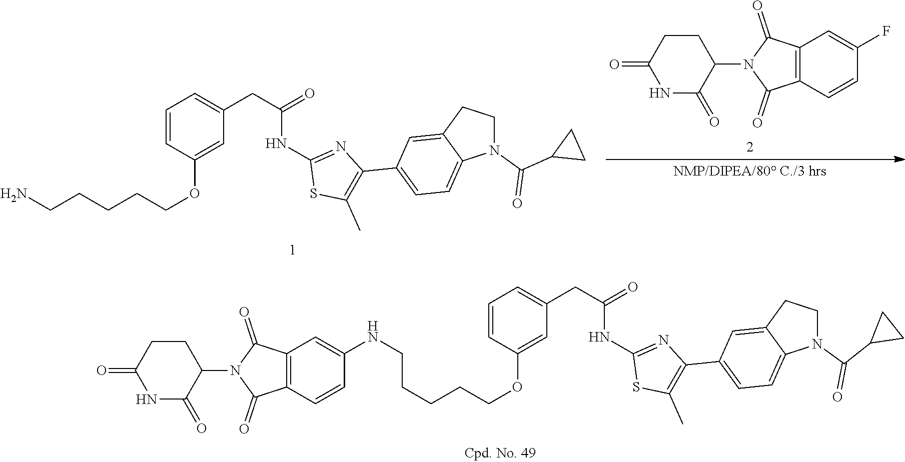 [SMILES: Cc1sc(NC(=O)Cc2cccc(OCCCCCN)c2)nc1-c1ccc2c(c1)CCN2C(=O)C1CC1 ; O=C1CCC(N2C(=O)c3ccc(F)cc3C2=O)C(=O)N1 ; Cc1sc(NC(=O)Cc2cccc(OCCCCCNc3ccc4c(c3)C(=O)N(C3CCC(=O)NC3=O)C4=O)c2)nc1-c1ccc2c(c1)CCN2C(=O)C1CC1]

### Step 1. N-(4-(1-(cyclopropanecarbonyl)indolin-5-yl)-5-methylthiazol-2-yl)-2-(3-(5-(2-(2,6-dioxopiperidin-3-yl)-1,3-dioxoisoindolin-5-ylamino)pentyloxy)phenyl)acetamide

A solution of 2-(3-(5-aminopentyloxy)phenyl)-N-(4-(1-(cyclopropanecarbonyl)indolin-5-yl)-5-methylthiazol-2-yl) acetamide (0.12 g, 0.23 mmol), 2-(2,6-dioxopiperidin-3-yl)-5-fluoroisoindoline-1,3-dione (0.16 g, 0.58 mmol) and N,N-diisopropylethylamine (74.8 mg, 0.58 mmol) in 1-methylpyrrolidin-2-one (5 mL) was stirred at 80° C. for 3 hrs. The mixture was cooled to room temperature, diluted with water (10 mL), extracted with dichloromethane (20 mL×3), washed with brine (20 mL), dried over anhydrous sodium sulfate, filtered and concentrated in vacuo. The residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15 min) to give N-(4-(1-(cyclopropanecarbonyl)indolin-5-yl)-5-methylthiazol-2-yl)-2-(3-(5-(2-(2,6-dioxopiperidin-3-yl)-1,3-dioxoisoindolin-5-ylamino)pentyloxy)phenyl)acetamide (16.5 mg, 9.2% yield) as a yellow solid. ¹H NMR (400 MHz, DMSO-d₆): δ 12.28 (s, 1H), 11.06 (s, 1H), 8.06 (d, J=7.6 Hz, 1H), 7.56 (d, J=8.4 Hz, 1H), 7.49 (s, 1H), 7.42 (d, J=8.4 Hz, 1H), 7.22 (t, J=7.6 Hz, 1H), 7.09 (t, J=5.2 Hz, 1H), 6.94 (s, 1H), 6.90-6.81 (m, 5.02 (dd, J=12.8 Hz, 5.2 Hz, 1H), 4.32 (brs, 2H), 3.96 (t, J=6.0 Hz, 2H), 3.69 (s, 2H), 3.28-3.17 (m, 2.91-2.82 (m, 1H), 2.59-2.53 (m, 2H), 2.43 (s, 3H), 2.00-1.97 (m, 2H), 1.79-1.73 (m, 2H), 1.67-1.55 (m, 2H), 1.52-1.48 (m, 2H), 0.87-0.83 (m, 4H). MS (ESI) m/z: 775.3 [M+H]⁺.

### Example 49

### Synthesis of N-(4-(1-(cyclopropanecarbonyl)indolin-5-yl)-5-methylthiazol-2-yl)-2-(3-(5-(2-(2,6-dioxopiperidin-3-yl)-1,3-dioxoisoindolin-5-ylamino)-3,3-dimethylpentyloxy)phenyl)acetamide formic acid (Cpd. No. 51)

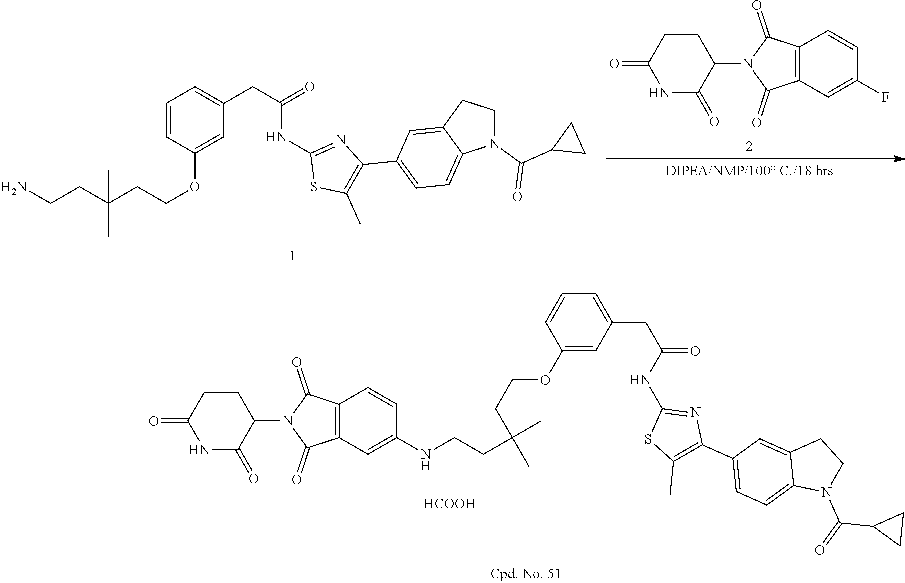 [SMILES: Cc1sc(NC(=O)Cc2cccc(OCCC(C)(C)CCN)c2)nc1-c1ccc2c(c1)CCN2C(=O)C1CC1 ; O=C1CCC(N2C(=O)c3ccc(F)cc3C2=O)C(=O)N1 ; Cc1sc(NC(=O)Cc2cccc(OCCC(C)(C)CCNc3ccc4c(c3)C(=O)N(C3CCC(=O)NC3=O)C4=O)c2)nc1-c1ccc2c(c1)CCN2C(=O)C1CC1 ; "HCOOH"]

### Step 1. N-(4-(1-(cyclopropanecarbonyl)indolin-5-yl)-5-methylthiazol-2-yl)-2-(3-(5-(2-(2,6-dioxopiperidin-3-yl)-1,3-dioxoisoindolin-5-ylamino)-3,3-dimethylpentyloxy)phenyl)acetamide formic acid

A solution of 2-(3-(5-amino-3,3-dimethylpentyloxy)phenyl)-N-(4-(1-(cyclopropanecarbonyl)indolin-5-yl)-5-methylthiazol-2-yl)acetamide (65.0 mg, 0.12 mmol), 2-(2,6-dioxopiperidin-3-yl)-5-fluoroisoindoline-1,3-dione (16.4 mg, 0.59 mmol) and N,N-diisopropylethylamine (77.0 mg, 0.59 mmol) in 1-methylpyrrolidin-2-one (1 mL) was stirred at 100° C. for 18 hrs. The mixture was diluted with water (10 mL), extracted with dichloromethane (20 mL×3), washed with brine (20 mL), dried over anhydrous sodium sulfate, filtered and concentrated in vacuo. The residue was purified by prep-HPLC (Column: Welch Xtimate 21.2*250 mm C18, 10 μm, Mobile Phase: A: water (0.01% formic acid) B: acetonitrile; B %: 30%-70% in 15 min) to give N-(4-(1-(cyclopropanecarbonyl)indolin-5-yl)-5-methylthiazol-2-yl)-2-(3-(5-(2-(2,6-dioxopiperidin-3-yl)-1,3-dioxoisoindolin-5-ylamino)-3,3-dimethylpentyloxy)phenyl)acetamide formic acid (3.0 mg, 3.0% yield) as a yellow solid. ¹H NMR (400 MHz, DMSO-d₆): δ 12.29 (s, 1H), 11.06 (s, 1H), 8.46 (s, 1H), 8.06 (d, J=9.2 Hz, 1H), 7.55 (d, J=8.0 Hz, 1H), 7.49 (s, 1H), 7.41 (d, J=8.8 Hz, 1H), 7.23 (t, J=8.0 Hz, 1-1), 7.04 (t, J=4.8 Hz, 1H), 6.95 (d, J=1.6 Hz, 1H), 6.91 (s, 1H), 6.87 (d, J=7.6 Hz, 1H), 6.85 (s, 1H), 6.82 (s, 2H), 5.02 (dd, J=12.8 Hz, 5.2 Hz, 1H), 4.31 (t, J=8.0 Hz, 2-1), 4.03 (t, J=7.2 Hz, 2H), 3.69 (s, 2H), 3.25-3.14 (m, 2.91-2.82 (m, 1H), 2.58-2.52 (m, 2H), 2.40 (s, 3H), 2.02-1.93 (m, 2H), 1.75 (t, J=7.0 Hz, 2H), 1.59 (t, J=8.0 Hz, 2H), 1.02 (s, 6H), 0.87-0.82 (m, 4H). MS (ESI) m/z: 802.6 [M+H]⁺.

### Example 50

### Synthesis of 4-(1-(cyclopropanecarbonyl)indolin-5-yl)-N-(3-(2-(2-((2-(2,6-dioxopiperidin-3-yl)-1,3-dioxoisoindolin-4-yl)amino)ethoxy)ethoxy)benzyl)-5-methylthiazole-2-carboxamide (Cpd. No. 56)

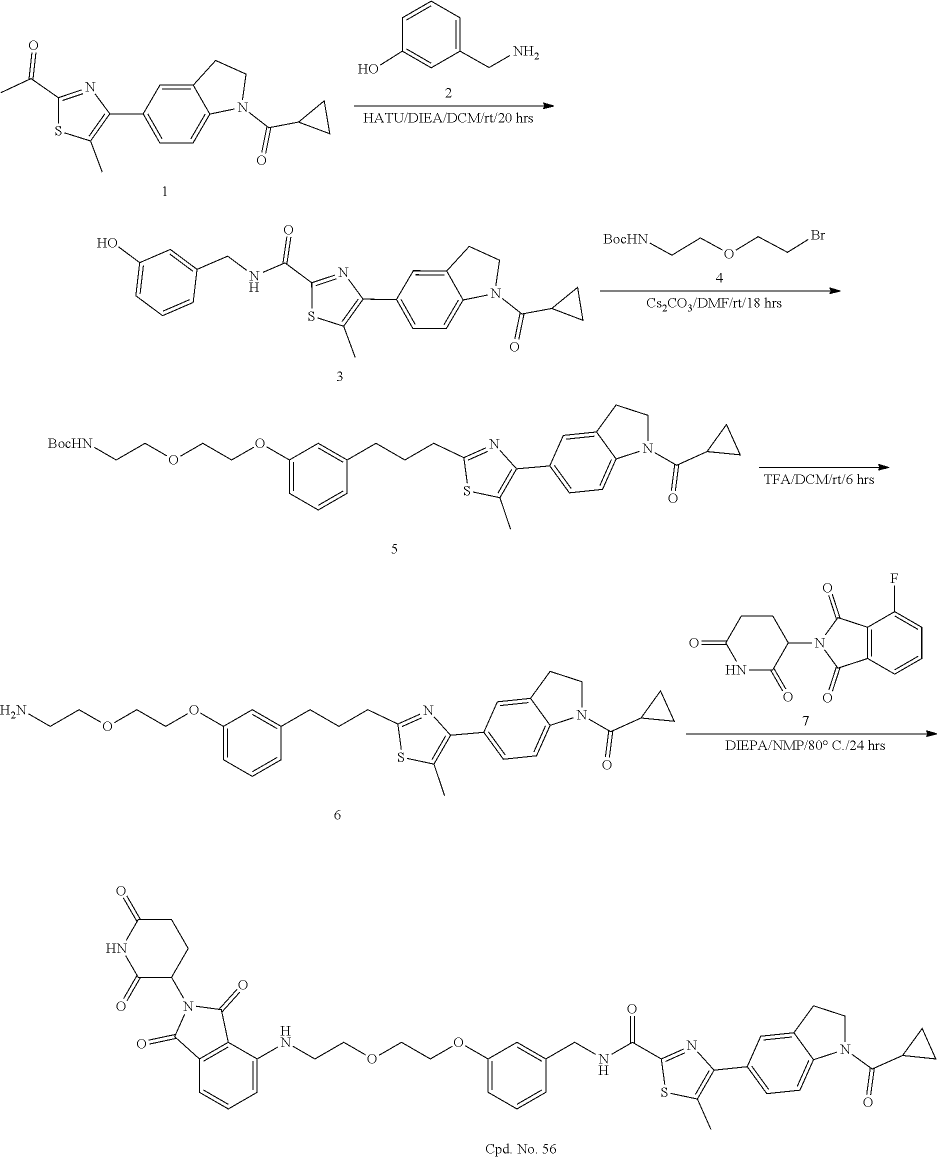

### Step 1. 4-(1-(cyclopropanecarbonyl)indolin-5-yl)-N-(3-hydroxybenzyl)-5-methylthiazole-2-carboxamide

A solution of 4-(1-(cyclopropanecarbonyl)indolin-5-yl)-5-methylthiazole-2-carboxylic acid (1.00 g, 3.05 mmol), N,N-diisopropylethylamine (1.58 g, 12.2 mmol), 2-(7-azabenzotriazol-1-yl)-N,N,N′,N′-tetramethyluronium hexafluorophosphate (1.74, 4.57 mmol) and 3-(aminomethyl)phenol (0.38 g, 3.1 mmol) in dichloromethane (50 mL) was stirred at room temperature for 20 hours. The reaction was quenched with water (80 mL), extracted with dichloromethane (70 mL×3), washed with brine (40 mL), dried over anhydrous sodium sulfate, filtered and concentrated in vacuo. The residue was purified by flash column chromatography (ethyl acetate/petroleum ether=2/1) to give 4-(1-(cyclopropanecarbonyl)indolin-5-yl)-N-(3-hydroxybenzyl)-5-methylthiazole-2-carboxamide (0.30 g, 23% yield) as a yellow solid. MS (ESI) m/z: 433.9 [M+H]⁺.

### Step 2. tert-butyl (2-(2-(3-((4-(1-(cyclopropanecarbonyl)indolin-5-yl)-5-methylthiazole-2-carboxamido)methyl)phenoxy)ethoxy)ethyl)carbamate

A mixture of 4-(1-(cyclopropanecarbonyl)indolin-5-yl)-N-(3-hydroxybenzyl)-5-methylthiazole-2-carboxamide (0.28 g, 0.65 mmol), tert-butyl (2-(2-bromoethoxy)ethyl)carbamate (0.17 g, 0.65 mmol) and cesium carbonate (0.63 g, 1.9 mmol) in AN-dimethylformamide (15 mL) was stirred at room temperature for 18 hrs. The mixture was diluted with water (80 mL), extracted with dichloromethane (80 mL×2), washed with brine (40 mL), dried over anhydrous sodium sulfate, filtered and concentrated in vacuo. The residue was purified by flash column chromatography (ethyl acetate/petroleum ether=2/1) to give tert-butyl (2-(2-(3-((4-(1-(cyclopropanecarbonyl) indolin-5-yl)-5-methylthiazole-2-carboxamido)methyl)phenoxy)ethoxy)ethyl)carbamate (0.25 g, 62% yield) as a white solid. MS (ESI) m/z: 621.0 [M+H]⁺.

### Step 3. N-(3-(2-(2-aminoethoxy)ethoxy)benzyl)-4-(1-(cyclopropanecarbonyl)indolin-5-yl)-5-methylthiazole-2-carboxamide

A solution of tert-butyl (2-(2-(3-((4-(1-(cyclopropanecarbonyl)indolin-5-yl)-5-methylthiazole-2-carboxamido)methyl)phenoxy)ethoxy)ethyl)carbamate (0.20 g, 0.32 mmol) and trifluoroacetic acid (6 mL) in dichloromethane (10 mL) was stirred at room temperature for 6 hrs. The solvent was removed in vacuo and the residue was purified by prep-HPLC (Column: Welch Xtimate 21.2*250 mm C18, 10 pin, Mobile Phase: A: water (10 mmol/L ammonium bicarbonate) B: acetonitrile; B %: 30%-70% in 15 min) to give A-(3-(2-(2-aminoethoxy)ethoxy)benzyl)-4-(1-(cyclopropanecarbonyl)indolin-5-yl)-5-methylthiazole-2-carboxamide (0.17 g, 100% yield) as a white solid. MS (ESI) m/z: 521.2 [M+H]⁺.

### Step 4. 4-(1-(cyclopropanecarbonyl)indolin-5-yl)-N-(3-(2-(2-((2-(2,6-dioxopiperidin-3-yl)-1,3-dioxoisoindolin-4-yl)amino)ethoxy)ethoxy)benzyl)-5-methylthiazole-2-carboxamide

A solution of N-(3-(2-(2-aminoethoxy)ethoxy)benzyl)-4-(1-(cyclopropanecarbonyl)indolin-5-yl)-5-methyl thiazole-2-carboxamide (0.15 g, 0.29 mmol), 2-(2,6-dioxopiperidin-3-yl)-4-fluoroisoindoline-1,3-dione (0.40 g, 1.4 mmol) and N,N-diisopropylethylamine (0.19 g, 1.4 mmol) in 1-methylpyrrolidin-2-one (6 mL) was stirred at 80° C. for 24 hrs. The mixture was cooled to room temperature, diluted with water (20 mL), extracted with dichloromethane (30 mL×3), washed with brine (20 mL), dried over anhydrous sodium sulfate, filtered and concentrated in vacuo. The residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in min) to give 4-(1-(cyclopropanecarbonyl)indolin-5-yl)-N-(3-(2-(2-((2-(2,6-dioxopiperidin-3-yl)-1,3-dioxoisoindolin-4-yl)amino)ethoxy)ethoxy)benzyl)-5-methylthiazole-2-carboxamide (58.5 mg, 26% yield) as a yellow solid. ¹H NMR (400 MHz, DMSO-d₆): δ 11.09 (s, 1H), 9.27 (t, J=6.8 Hz, 1H), 8.09 (d, J=8.0 Hz, 1H), 7.65 (s, 1H), 7.57-7.53 (m, 2H), 7.23 (t, J=8.4 Hz, 1H), 7.13 (d, J=8.4 Hz, 1H), 7.01 (d, J=7.2 Hz, 1H), 6.90-6.88 (m, 2H), 6.82 (d, J=7.2 Hz, 1H), 6.63 (t, J=5.6 Hz, 1H), 5.04 (dd, J=12.8 Hz, 5.6 Hz, 1H), 4.41 (d, J=6.4 Hz, 2H), 4.34 (t, J=6.4 Hz, 2H), 4.07 (t, J=4.0 Hz, 2H), 3.77 (t, J=4.8 Hz, 2H), 3.67 (t, J=5.2 Hz, 2H), 3.47 (q, J=5.6 Hz, 2-1), 3.24 (t, J=6.8 Hz, 2H), 2.88-2.84 (m, 1H), 2.66-2.52 (m, 5H), 2.07-1.97 (m, 2H), 0.89-0.86 (m, 4H). MS (ESI) m/z: 777.7 [M+H]⁺.

### Example 51

### Synthesis of N-(3-(2-(2-(2-(2,6-dioxopiperidin-3-yl)-1,3-dioxoisoindolin-4-ylamino)ethoxy)ethoxy)benzyl)-5-methyl-4-(1-(1-methyl-1H-imidazole-5-carbonyl)indolin-5-yl)thiazole-2-carboxamide (Cpd. No. 57)

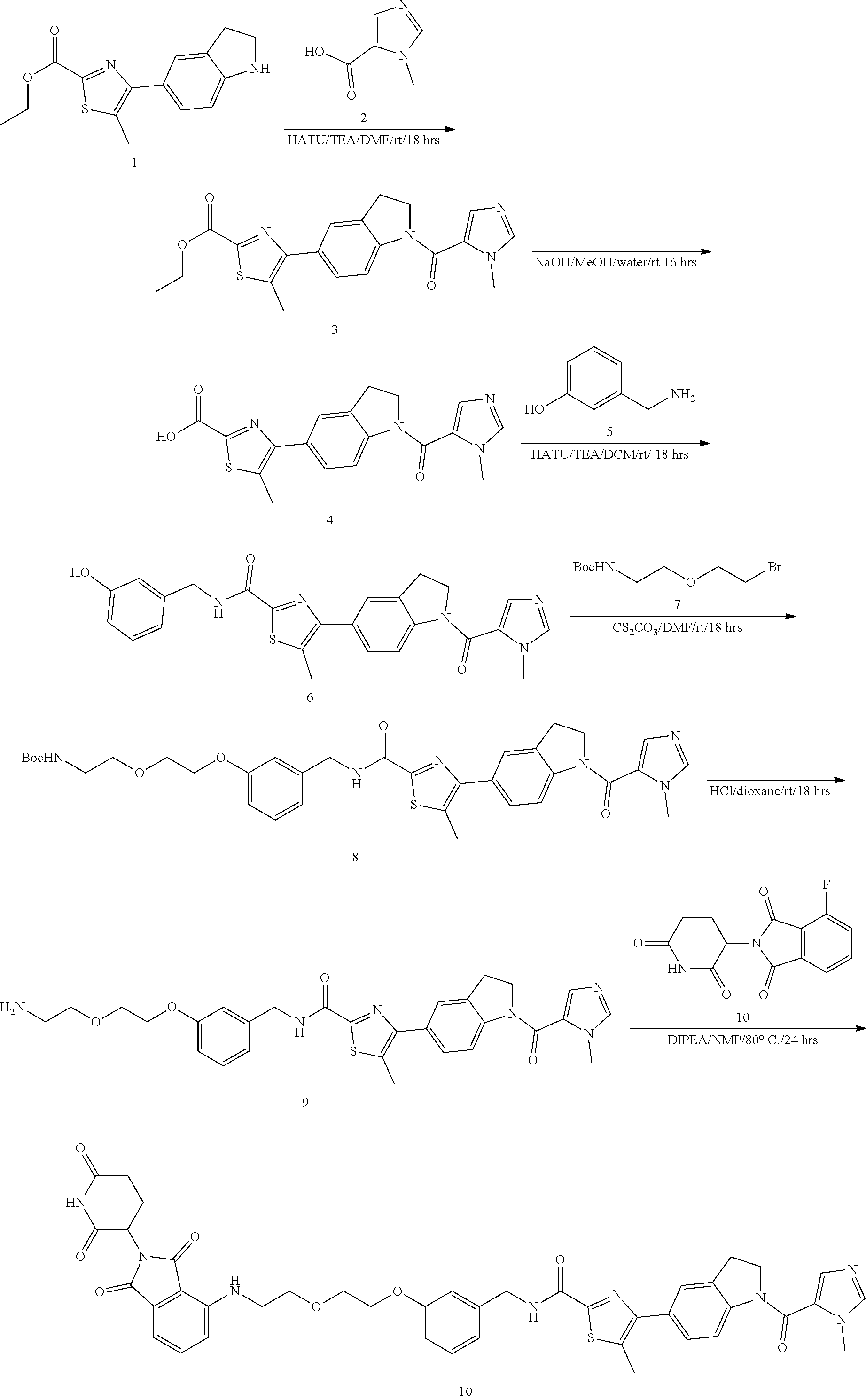

### Step 1. ethyl 5-methyl-4-(1-(1-methyl-1H-imidazole-5-carbonyl)indolin-5-yl)thiazole-2-carboxylate

A solution of 1-methyl-1H-imidazole-5-carboxylic acid (1.00 g, 7.93 mmol) ethyl 4-(indolin-5-yl)-5-methylthiazole-2-carboxylate (2.30 g, 7.98 mmol), 2-(7-azabenzotriazol-1-yl)-N,N,N′,N′-tetramethyluronium hexafluorophosphate (3.64 g, 9.57 mmol) and triethylamine (2.42 g, 23.9 mmol) in N, N-dimethylformamide (20 mL) was stirred at room temperature for 18 hrs. The mixture was diluted with water (40 mL) and filtered. The filter cake was dried to give ethyl 5-methyl-4-(1-(1-methyl-1H-imidazole-5-carbonyl)indolin-5-yl)thiazole-2-carboxylate (2.50 g, 79.5% yield) as a gray solid. MS (ESI) m/z: 397.4 [M+H]⁺.

### Step 2. 5-methyl-4-(1-(1-methyl-1H-imidazole-5-carbonyl)indolin-5-yl)thiazole-2-carboxylic acid

A solution of ethyl 5-methyl-4-(1-(1-methyl-1H-imidazole-5-carbonyl)indolin-5-yl)thiazole-2-carboxylate (2.50 g, 6.31 mmol) and sodium hydroxide (1.01 g, 25.2 mmol) in methanol (30 mL) and water (20 mL) was stirred at room temperature for 16 hrs. The mixture was diluted with water (20 mL), acidified with 2 N hydrochloric acid to pH=3-4, filtered. The cake was dried to give 5-methyl-4-(1-(1-methyl-1H-imidazole-5-carbonyl)indolin-5-yl) thiazole-2-carboxylic acid (1.95 g, 83.9% yield) as a gray solid. MS (ESI) m/z: 369.2 [M+H]⁺.

### Step 3. N-(3-hydroxybenzyl)-5-methyl-4-(1-(1-methyl-1H-imidazole-5-carbonyl)indolin-5-yl)thiazole-2-carboxamide

A solution of 5-methyl-4-(1-(1-methyl-1H-imidazole-5-carbonyl)indolin-5-yl)thiazole-2-carboxylic acid (1.80 g, 4.89 mmol), 3-(aminomethyl)phenol (0.60 g, 4.9 mmol), 2-(7-azabenzotriazol-1-yl)-N,N,N′,N′-tetramethyluroniumhexafluorophosphate (2.78 g, 7.33 mmol) and triethylamine (1.48 g, 14.7 mmol) in dichloromethane (30 mL) was stirred at room temperature for 18 hrs. Dichloromethane was removed in vacuo and then the residue was suspended in methanol/water (20 mL/20 mL) and filtered. The filter cake was dried to give N-(3-hydroxybenzyl)-5-methyl-4-(1-(1-methyl-1H-imidazole-5-carbonyl)indolin-5-yl)thiazole-2-carboxamide (2.20 g, 95.1% yield) as a yellow solid. MS (ESI) m/z 474.3[M+H]⁺.

### Step 4. tert-butyl (2-(2-(3-((5-methyl-4-(1-(1-methyl-1H-imidazole-5-carbonyl)indolin-5-yl)thiazole-2-carboxamido)methyl)phenoxy)ethoxy)ethyl)carbamate

A solution of A-(3-hydroxybenzyl)-5-methyl-4-(1-(1-methyl-1H-imidazole-5-carbonyl)indolin-5-yl)thiazole-2-carboxamide (1.00 g, 2.11 mmol), tert-butyl 2-(2-bromoethoxy)ethylcarbamate (0.57 g, 2.12 mmol) and cesium carbonate (1.38 g, 4.22 mmol) in N,N-dimethylformamide (20 mL) was stirred at room temperature for 18 hrs. The mixture was diluted with water (40 mL), extracted with dichloromethane (30 mL×2), washed with brine (20 mL), dried over anhydrous sodium sulfate, filtered and concentrated in vacuo. The residue was purified by flash column chromatography (dichloromethane/methanol=95/5) to give tert-butyl 2-(2-(3-((5-methyl-4-(1-(1-methyl-1H-imidazole-5-carbonyl)indolin-5-yl)thiazole-2-carboxamido)methyl)phenoxy)ethoxy)ethylcarbamate (0.95 g, 68% yield) as a yellow solid. MS (ESI) m/z: 661.3 [M+H]⁺.

### Step 5. N-(3-(2-(2-aminoethoxy)ethoxy)benzyl)-5-methyl-4-(1-(1-methyl-1H-imidazole-5-carbonyl)indolin-5-yl)thiazole-2-carboxamide

A solution of tert-butyl 2-(2-(3-((5-methyl-4-(1-(1-methyl-1H-imidazole-5-carbonyl)indolin-5-yl)thiazole-2-carboxamido)methyl)phenoxy)ethoxy)ethylcarbamate (0.45 g, 0.68 mmol) in hydrochloric acid (2 M in dioxane, 20 mL) was stirred at room temperature for 18 hrs. The solvent was removed in vacuo and the residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15 min) to give N-(3-(2-(2-aminoethoxy)ethoxy)benzyl)-5-methyl-4-(1-(1-methyl-1H-imidazole-5-carbonyl)indolin-5-yl)thiazole-2-carboxamide (0.18 g, 47% yield) as a yellow solid. MS (ESI) m/z: 561.2 [M+H]⁺.

### Step 6. N-(3-(2-(2-(2-(2,6-dioxopiperidin-3-yl)-1,3-dioxoisoindolin-4-ylamino)ethoxy)ethoxy)benzyl)-5-methyl-4-(1-(1-methyl-1H-imidazole-5-carbonyl)indolin-5-yl)thiazole-2-carboxamide

A solution of N-(3-(2-(2-aminoethoxy)ethoxy)benzyl)-5-methyl-4-(1-(1-methyl-1H-imidazole-5-carbonyl)indolin-5-yl)thiazole-2-carboxamide (80.0 mg, 0.14 mmol), 2-(2,6-dioxopiperidin-3-yl)-4-fluoroisoindoline-1,3-dione (47.3 mg, 0.17 mmol) and N, N-diisopropylethylamine (36.9 mg, 0.29 mmol) in 1-methylpyrrolidin-2-one (2 mL) was stirred at 80° C. for 24 hrs. The mixture was diluted with water (20 mL), extracted with dichloromethane (30 mL×3), washed with brine (20 mL), dried over anhydrous sodium sulfate, filtered and concentrated in vacuo. The residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15 min) to give N-(3-(2-(2-(2-(2,6-dioxopiperidin-3-yl)-1,3-dioxoisoindolin-4-ylamino)ethoxy)ethoxy)benzyl)-5-methyl-4-(1-(1-methyl-1H-imidazole-5-carbonyl)indolin-5-yl)thiazole-2-carboxamide (41.6 mg, 36% yield) as a yellow solid. ¹H NMR (400 MHz, DMSO-d₆): δ 11.10 (s, 1H), 9.29 (t, J=6.4 Hz, 1H), 8.09 (d, J=8.4 Hz, 1H), 7.85 (s, 1H), 7.70 (s, 1H), 7.61-7.60 (m, 2H), 7.54 (t, J=8.0 Hz, 1H), 7.22 (t, J=8.0 Hz, 1H), 7.13 (d, J=8.8 Hz, 1H), 7.01 (d, J=7.2 Hz, 1H), 6.90-6.89 (m, 2H), 6.82 (dd, J=7.6 Hz, 2 Hz, 1H), 6.64 (t, J=5.6 Hz, 1H), 5.05 (dd, J=12.8 Hz, 5.2 Hz, 1H), 4.43 (d, J=6.0 Hz, 2H), 4.37 (t, J=8.4 Hz, 2H), 4.07 (t, J=4.0 Hz, 2H), 3.80 (s, 3H), 3.77 (t, J=4.4 Hz, 2H), 3.68 (t, J=5.2 Hz, 2H), 3.48 (q, J=5.2 Hz, 2H), 3.20 (t, J=8.0 Hz, 2H), 2.88-2.82 (m, 1H), 2.67 (s, 3H), 2.62-2.58 (m, 2H), 2.03-1.97 (m, 1H). MS (ESI) m/z: 817.2 [M+H]⁺.

### Example 52

### Synthesis of N-(3-(2-(2-(2-(2,6-dioxopiperidin-3-yl)-1,3-dioxoisoindolin-5-ylamino)ethoxy)ethoxy)benzyl)-5-methyl-4-(1-(1-methyl-1H-imidazole-5-carbonyl)indolin-5-yl)thiazole-2-carboxamide (Cpd. No. 58)

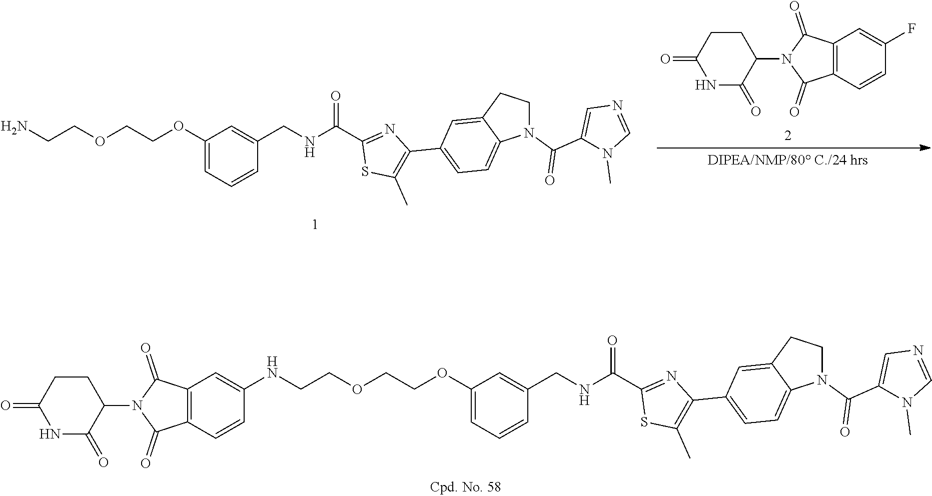 [SMILES: Cc1sc(C(=O)NCc2cccc(OCCOCCN)c2)nc1-c1ccc2c(c1)CCN2C(=O)c1cncn1C ; O=C1CCC(N2C(=O)c3ccc(F)cc3C2=O)C(=O)N1 ; Cc1sc(C(=O)NCc2cccc(OCCOCCNc3ccc4c(c3)C(=O)N(C3CCC(=O)NC3=O)C4=O)c2)nc1-c1ccc2c(c1)CCN2C(=O)c1cncn1C]

### Step 1. N-(3-(2-(2-(2-(2,6-dioxopiperidin-3-yl)-1,3-dioxoisoindolin-5-ylamino)ethoxy)ethoxy)benzyl)-5-methyl-4-(1-(1-methyl-1H-imidazole-5-carbonyl)indolin-5-yl)thiazole-2-carboxamide (Cpd. No. 58)

A solution of N-(3-(2-(2-aminoethoxy)ethoxy)benzyl)-5-methyl-4-(1-(1-methyl-1H-imidazole-5-carbonyl)indolin-5-yl)thiazole-2-carboxamide (80.0 mg, 0.14 mmol), 2-(2,6-dioxopiperidin-3-yl)-5-fluoroisoindoline-1,3-dione (0.12 g, 0.43 mmol) and N,N-diisopropylethylamine (55.3 mg, 0.43 mmol) in 1-methylpyrrolidin-2-one (2 mL) was stirred at 80° C. for 24 hrs. The mixture was diluted with water (20 mL), extracted with dichloromethane (30 mL×3), washed with brine (20 mL), dried over anhydrous sodium sulfate, filtered and concentrated in vacuo. The residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15 min) to give N-(3-(2-(2-(2-(2,6-dioxopiperidin-3-yl)-1,3-dioxoisoindolin-5-ylamino)ethoxy)ethoxy)benzyl)-5-methyl-4-(1-(1-methyl-1H-imidazole-5-carbonyl)indolin-5-yl)thiazole-2-carboxamide (10.0 mg, 8.6% yield) as a yellow solid. ¹H NMR (400 MHz, DMSO-d₆): δ 11.12 (s, 1H), 9.29 (t, J=6.0 Hz, 1H), 8.09 (d, J=8.4 Hz, 1H), 7.85 (s, 1H), 7.70 (s, 1H), 7.61-7.59 (m, 2H), 7.54 (d, J=8.4 Hz, 1H), 7.22 (t, J=8.0 Hz, 1H), 7.17 (t, J=5.6 Hz, 1H), 7.00 (d, J=1.6 Hz, 1H), 6.90-6.86 (m, 3H), 6.81 (d, J=7.6 Hz, 1H), 5.02 (dd, J=12.8 Hz, 5.2 Hz, 1H), 4.42 (d, J=6.0 Hz, 2H), 4.37 (t, J=8.4 Hz, 2H), 4.08 (t, J=4.8 Hz, 2H), 3.80 (s, 3H), 3.76 (t, J=4.4 Hz, 2H), 3.65 (t, J=5.2 Hz, 2H), 3.38 (q, J=4.8 Hz, 2H), 3.20 (t, J=8.0 Hz, 2H), 2.88-2.82 (m, 1H), 2.62 (s, 3H), 2.60-2.55 (m, 2H), 2.03-1.97 (m, 1H). MS (ESI) m/z: 817.2 [M+H]⁺.

### Example 53

### Synthesis of N-((3-(2-(2-(2-(2,6-dioxopiperidin-3-yl)-1-oxoisoindolin-5-ylamino)ethoxy)ethoxy)phenyl)methyl)-5-methyl-4-(1-(1-methyl-1H-imidazole-5-carbonyl)indolin-5-yl)thiazole-2-carboxamide (Cpd. No. 59)

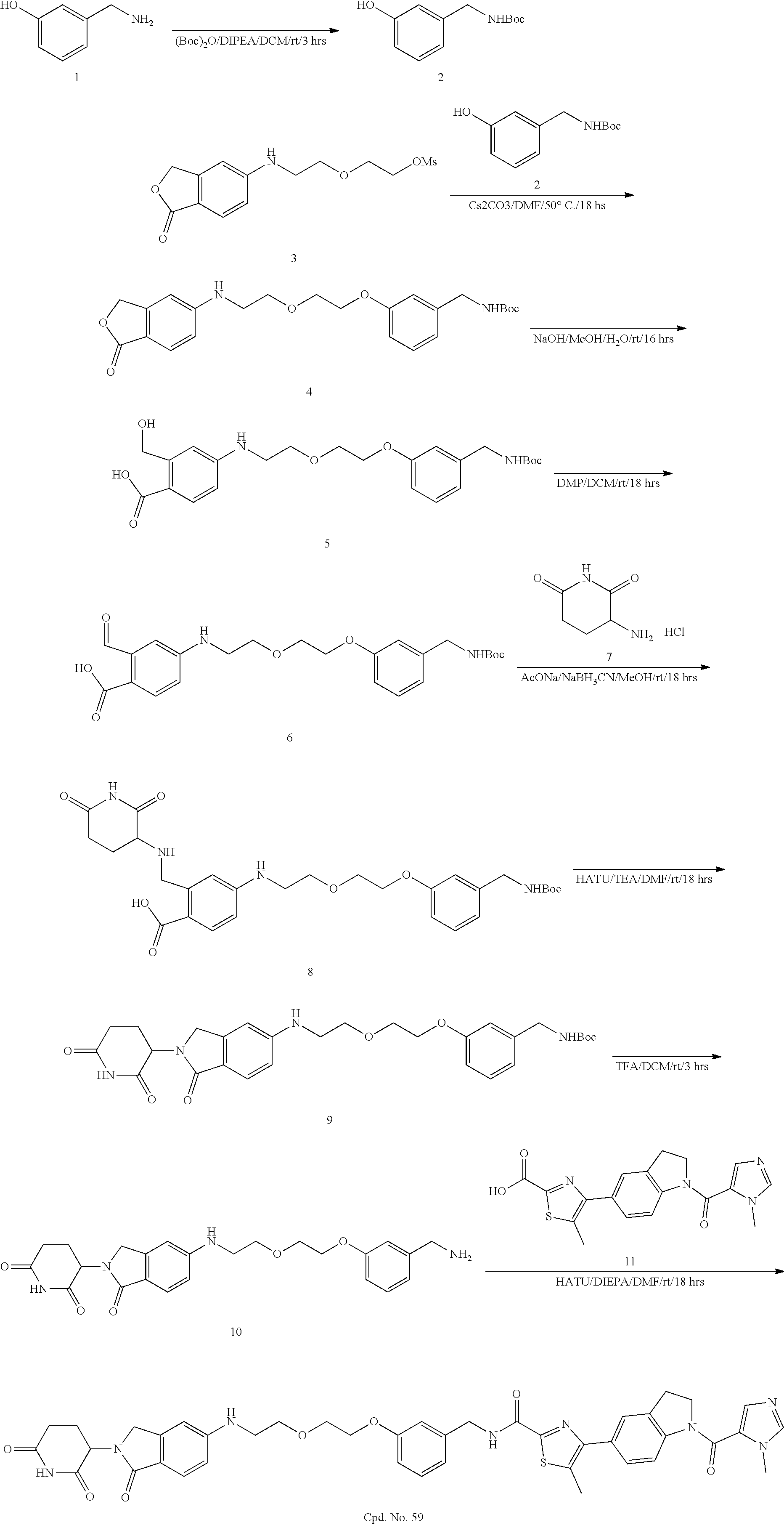

### Step 1. tert-butyl (3-hydroxybenzyl)carbamate

A mixture of 3-(aminomethyl)phenol (3.00 g, 24.4 mmol), di-tert-butyl dicarbonate (5.31 g, 24.4 mmol) and diisopropylethylamine (9.45 g, 73.1 mmol) in dichloromethane (50 mL) was stirred at room temperature for 3 hrs. The mixture was diluted with water (30 mL), extracted with dichloromethane (50 mL), washed with brine (30 mL×2), dried over anhydrous sodium sulfate, filtered and concentrated in vacuo to give tert-butyl (3-hydroxybenzyl)carbamate (3.70 g, 68.0% yield) as a corlourless oil. MS (ESI) m/z: 224.3 [M+H]⁺.

### Step 2. tert-butyl (3-(2-(2-(1-oxo-1,3-dihydroisobenzofuran-5-ylamino)ethoxy)ethoxy)phenyl) methylcarbamate

A solution of 2-(2-(1-oxo-1,3-dihydroisobenzofuran-5-ylamino)ethoxy)ethyl methanesulfonate (4.50 g, 14.3 mmol), tert-butyl (3-hydroxybenzyl)carbamate (3.19 g, 14.3 mmol) and cesium carbonate (13.95 g, 42.8 mmol) in N,N-dimethylformamide (40 mL) was stirred at 50° C. for 18 hrs. The mixture was cooled to room temperature, diluted with water (50 mL), extracted with dichloromethane (50 mL×3), washed with brine (30 mL×2), dried over anhydrous sodium sulfate, filtered and concentrated in vacuo to give tert-butyl (3-(2-(2-(1-oxo-1,3-dihydroisobenzofuran-5-ylamino)ethoxy)ethoxy)phenyl)methylcarbamate (5.60 g, 88.7% yield) as a yellow oil. MS (ESI) m/z: 443.4 [M+H]⁺.

### Step 3. 4-(2-(2-(3-((tert-butoxycarbonyl)methyl)phenoxy)ethoxy)ethylamino)-2-(hydroxymethyl) benzoic acid

A solution of (3-(2-(2-(i-oxo-1,3-dihydroisobenzofuran-5-ylamino)ethoxy)ethoxy)phenyl)methylcarbamate (5.60 g, 12.7 mmol) and sodium hydroxide (2.53 g, 63.3 mmol) in methanol (20 mL) and water (20 mL) was stirred at room temperature for 16 hrs. The mixture was diluted with water (20 mL), acidified with hydrochloric acid (2 M) aqueous solution to pH=3, extracted with dichloromethane (80 mL×5), dried over anhydrous sodium sulfate, filtered and concentrated in vacuo. The residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15 min) to give 4-(2-(2-(3-((tert-butoxycarbonyl)methyl) phenoxy)ethoxy)ethylamino)-2-(hydroxymethyl)benzoic acid (4.10 g, 72% yield) as a brown oil. MS (ESI) m/z: 461.4 [M+H]⁺.

### Step 4. 4-(2-(2-(3-((tert-butoxycarbonyl)methyl)phenoxy)ethoxy)ethylamino)-2-formylbenzoic acid

A solution of 4-(2-(2-(3-(tert-butoxycarbonyl)methyl)phenoxy)ethoxy)ethylamino)-2-(hydroxymethyl)benzoic acid (1.00 g, 2.24 mmol) and Dess-martin reagent (1.38 g, 3.26 mmol) in dichloromethane (40 mL) was stirred at room temperature for 18 hrs. The mixture was diluted with water (60 mL), extracted with dichloromethane (80 mL×3), dried over anhydrous sodium sulfate, filtered and concentrated in vacuo. The residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15 min) to give 4-(2-(2-(3-((tert-butoxycarbonyl)methyl)phenoxy)ethoxy)ethylamino)-2-formylbenzoic acid (0.26 g, 26% yield) as a white solid. MS (ESI) m/z: 359.3 [M+H−100]⁺.

### Step 5. 4-(2-(2-(3-((tert-butoxycarbonyl)methyl)phenoxy)ethoxy)ethylamino)-2-((2,6-dioxo piperidin-3-ylamino)methyl)benzoic acid

To a solution of 4-(2-(2-(3-((tert-butoxycarbonyl)methyl)phenoxy)ethoxy)ethylamino)-2-formylbenzoic acid (0.25 g, 0.56 mmol) and 3-aminopiperidine-2,6-dione hydrochloride (0.13 g, 0.82 mmol) in methanol (25 mL), was added sodium acetate (0.13 g, 1.6 mmol) and sodium cyanborohydride (0.10 g, 1.6 mmol) at room temperature. After addition, the mixture was stirred at room temperature for 18 hrs. The mixture was diluted with water (35 mL), extracted with dichloromethane (40 mL×4), dried over anhydrous sodium sulfate, filtered and concentrated in vacuo. The residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15 min) to give 4-(2-(2-(3-((tert-butoxycarbonyl)methyl)phenoxy)ethoxy)ethylamino)-2-((2,6-dioxopiperidin-3-ylamino) methyl)benzoic acid (0.15 g, 48% yield) as a white solid. MS (ESI) m/z: 571.4 [M+H]⁺.

### Step 6. tert-butyl (3-(2-(2-((2-(2,6-dioxopiperidin-3-yl)-1-oxoisoindolin-5-yl)amino)ethoxy)ethoxy)benzyl)carbamate

A solution of 4-(2-(2-(3-((tert-butoxycarbonyl)methyl)phenoxy)ethoxy)ethylamino)-2-((2,6-dioxopiperidin-3-ylamino)methyl)benzoic acid (0.13 g, 0.23 mmol), 2-(7-azabenzotriazol-1-yl)-N,N,N′,N′-tetramethyluronium hexafluorophosphate (0.13 g, 0.34 mmol) and triethylamine (69.2 mg, 0.68 mmol) in N,N-dimethylformamide (4 mL) was stirred at room temperature for 18 hrs. The mixture was diluted with water (15 mL), extracted with dichloromethane (30 mL×3), dried over anhydrous sodium sulfate, filtered and concentrated in vacuo. The residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15 min) to give tert-butyl (3-(2-(2-((2-(2,6-dioxopiperidin-3-yl)-1-oxoisoindolin-5-yl)amino)ethoxy)ethoxy)benzyl)carbamate (60.0 mg, 47.7% yield) as a white solid. MS (ESI) m/z: 553.2 [M+H]⁺.

### Step 7. 3-(5-(2-(2-(3-(aminomethyl)phenoxy)ethoxy)ethylamino)-1-oxoisoindolin-2-yl)piperidine-2,6-dione

A solution of tert-butyl (3-(2-(2-((2-(2,6-dioxopiperidin-3-yl)-1-oxoisoindolin-5-yl)amino)ethoxy)ethoxy)benzyl)carbamate (60.0 mg, 0.11 mmol) in trifluoroacetic acid (2 mL) ans dichloromethane (2 mL) was stirred at room temperature for 3 brs. The solvent was removed in vacuo. The residue was dissolved in dichloromethane (50 mL), washed with saturated sodium carbonate aqueous solution, dried over anhydrous sodium sulfate, filtered and concentrated in vacuo to give 3-(5-(2-(2-(3-(aminomethyl)phenoxy)ethoxy)ethylamino)-1-oxoisoindolin-2-yl) piperidine-2,6-dione (42.1 mg, 85% yield) as a gray solid. MS (ESI) m/z: 453.1 [M+H]⁺.

### Step 8. N-((3-(2-(2-(2-(2,6-dioxopiperidin-3-yl)-1-oxoisoindolin-5-ylamino)ethoxy)ethoxy)phenyl)methyl)-5-methyl-4-(1-(1-methyl-1H-imidazole-5-carbonyl)indolin-5-yl)thiazole-2-carboxamide

A solution of 3-(5-(2-(2-(3-(aminomethyl)phenoxy)ethoxy)ethylamino)-1-oxoisoindolin-2-yl)piperidine-2,6-dione (40.0 mg, 0.088 mmol), 5-methyl-4-(1-(1-methyl-1H-imidazole-5-carbonyl)indolin-5-yl)thiazole-2-carboxylic acid (32.6 mg, 0.088 mmol), 2-(7-azabenzotriazol-1-yl)-N,N,N′,N′-tetramethyluronium hexafluorophosphate (40.3 mg, 0.11 mmol) and diisopropylethylamine (34.3 mg, 0.27 mmol) in N,N-dimethylformamide (2 mL) was stirred at room temperature for 18 hrs. The mixture was diluted with water (10 mL), extracted with dichloromethane (25 mL×3), dried over anhydrous sodium sulfate, filtered and concentrated in vacuo. The residue was purified by prep-HPLC (Column: Welch Xtimate 21.2*250 mm C18, 10 μm, Mobile Phase: A: water (10 mmol/L ammonium bicarbonate) B: acetonitrile; B %: 30%-70% in 15 ruin) to give N-((3-(2-(2-(2-(2,6-dioxopiperidin-3-yl)-1-oxoisoindolin-5-ylamino)ethoxy)ethoxy)phenyl)methyl)-5-methyl-4-(1-(1-methyl-1H-imidazole-5-carbonyl)indolin-5-yl)thiazole-2-carboxamide (18.2 mg, 25.6% yield) as a white solid. ¹H NMR (400 MHz, DMSO-d₆): δ 10.94 (s, 1H), 9.32 (t, J=6.4 Hz, 1H), 8.09 (d, J=8.8 Hz, 1H), 7.86 (s, 1H), 7.71 (s, 1H), 7.62-7.60 (m, 2H), 7.37 (d, J=8.0 Hz, 1H), 7.23 (t, J=8.4 Hz, 1H), 6.91-6.89 (m, 2H), 6.81 (d, J=7.6 Hz, 1H), 6.69-6.65 (m, 2H), 6.41 (t, J=5.6 Hz, 1H), 5.01 (dd, J=13.2, 4.8 Hz, 1H), 4.44-4.36 (m, 4.25-4.07 (m, 4H), 3.80 (s, 3H), 3.76 (t, J=4.4 Hz, 2H), 3.63 (t, J=5.6 Hz, 2H), 3.31-3.26 (m, 2H), 3.20 (t, J=8.4 Hz, 2H), 2.92-2.85 (m, 1H), 2.62 (s, 3H), 2.62-2.54 (m, 1H), 2.34-2.29 (m, 1H), 1.94-1.90 (m, 1H). MS (ESI) m/z: 803.3 [M+H]⁺.

### Example 54

### Synthesis of N-[4-(1-cyclopropanecarbonyl-2,3-dihydro-1H-indol-5-yl)-5-methyl-1,3-thiazol-2-yl]-2-[3-(4-{[2-(2,6-dioxopiperidin-3-yl)-1,3-dioxo-2,3-dihydro-1H-isoindol-4-yl]amino}butoxy)phenyl]acetamide (Cpd. No. 65)

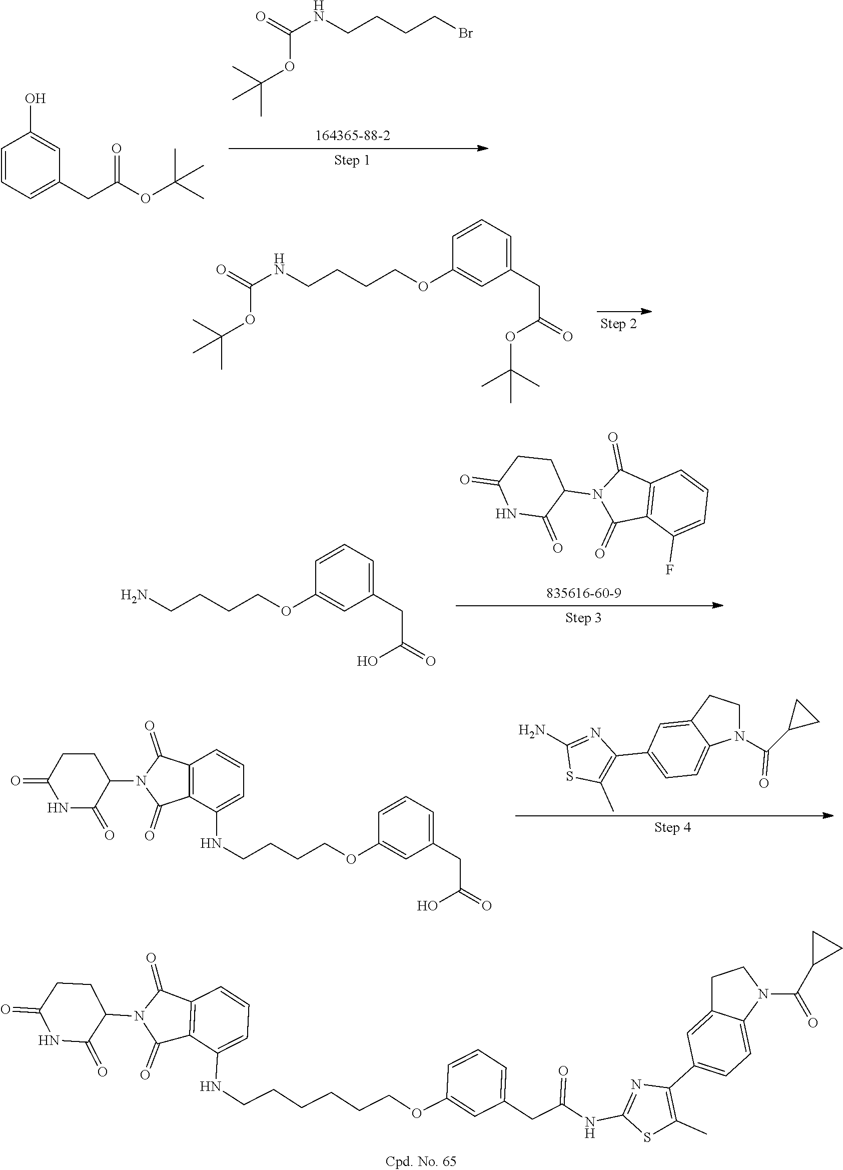

### Step 1: tert-butyl 2-[3-(4-{[(tert-butoxy)carbonyl]amino}butoxy)phenyl]acetate

A mixture of tert-butyl 2-(3-hydroxyphenyl)acetate (0.5 g, 2.281 mmol, 1.0 eq), 4-(Boc-amino)butyl bromide (0.575 g, 2.281 mmol, 1.0 eq) and cesium carbonate (2.229 g, 6.843 mmol, 3.0 eq) in N,N-dimethylformamide (16.3 ml, 0.14 M) was stirred at room temperature overnight. RM was evaporated and to the residue was added EtOAc. The mixture was filtered and the filter cake was washed thoroughly with EtOAc. The filtrate was evaporated to give 0.897 g (quant. yield) of the title compound as a brown oil. LCMS: [C₂₁H₃₃NO₅], desired mass=379.497, observed mass=380.25 [M+H], ¹H NMR (300 MHz, Chloroform-d) δ 7.24 (t, J=7.9 Hz, 1H), 6.89-6.77 (m, 3H), 4.63 (s, 1H), 3.99 (t, J=6.1 Hz, 2H), 3.51 (s, 2H), 3.22 (q, J=6.5 Hz, 2H), 1.83 (dt, J=13.0, 6.3 Hz, 2H), 1.76-1.63 (m, 2H), 1.47 (d, J=2.3 Hz, 18H).

### Step 2: 2-[3-(4-aminobutoxy)phenyl]acetic acid; trifluoroacetic acid

Tert-butyl 2-[3-(4-{[(tert-butoxy)carbonyl]amino}butoxy)phenyl]acetate (0.897 g, 2.281 mmol, 1.0 eq) was dissolved in a mixture of DCM (5.7 ml, 0.4 M) and TFA (7.802 g, 68.428 mmol, 30.0 eq), and it was stirred at rt for 1 h. UPLC analysis showed full conversion. It was evaporated on rotavap, dissolved a few times in DCM and then in ACN, followed by evaporation after every addition of the solvent and evaporated to dryness to get rid of remaining water. It was taken to the next step without purification. 1.062 g of the crude material as a viscous oil with quantitative yield was obtained. LCMS: [C₁₄H₁₈F₃NO₅], desired mass=337.295, observed mass=224.15 [M+H]⁺, ¹H NMR (300 MHz, DMSO) δ 7.71 (s, 3H), 7.27-7.15 (m, 1H), 6.93-6.79 (m, 3H), 5.27-5.13 (m, 1H), 3.97 (t, J=5.8 Hz, 2H), 3.56 (s, 2H), 2.99-2.81 (m, 2H), 1.89-1.59 (m, 4H).

### Step 3: 2-[3-(4-{[2-(2,6-dioxopiperidin-3-yl)-1,3-dioxo-2,3-dihydro-1H-isoindol-4-yl]amino}butoxy)phenyl]acetic acid

A mixture of 2-(2,6-Dioxo-piperidin-3-yl)-4-fluoroisoindoline-1,3-dione (1.315 g, 4.761 mmol, 2.1 eq), 2-[3-(4-aminobutoxy)phenyl]acetic acid; trifluoroacetic (1.062 g, 2.267 mmol, 1.0 eq) and DIPEA (2.053 ml, 11.788 mmol, 5.2 eq) in NMP (11.33 ml, 0.2 M) was stirred at 100° C. for overnight. UPLC analysis showed full conversion. DIPEA was evaporated, and the solution in NMP was injected on the column for RPFC. It was purified in acidic conditions with 0.1% FA and eluted with ACN:H₂O (10-54% of ACN) to give 590 mg (52% yield) of the title compound as a green solid. LCMS: [C₂₅H₂₅N₃O₇], desired mass=479.489, observed mass=480.15 [M+H]⁺, ¹H NMR (300 MHz, DMSO) δ 12.31 (s, 1H), 11.10 (s, 1H), 7.59 (dd, J=8.6, 7.1 Hz, 1H), 7.21 (td, J=7.8, 7.3, 1.7 Hz, 1H), 7.14 (d, J=8.6 Hz, 1H), 7.03 (d, J=7.0 Hz, 1H), 6.83 (s, 2H), 6.82-6.79 (m, 11), 6.64 (t, J=6.1 Hz, 1H), 5.06 (dd, J=12.8, 5.3 Hz, 1H), 4.01 (t, J=5.9 Hz, 2H), 3.52 (s, 2H), 3.45-3.38 (m, 2H), 2.98-2.82 (m, 1H), 2.64-2.54 (m, 2H), 2.12-1.96 (m, 1H), 1.88-1.69 (m, 4H).

### Step 4: N-[4-(1-cyclopropanecarbonyl-2,3-dihydro-1H-indol-5-yl)-5-methyl-1,3-thiazol-2-yl]-2-[3-(4-{[2-(2,6-dioxopiperidin-3-yl)-1,3-dioxo-2,3-dihydro-1H-isoindol-4-yl]amino}butoxy)phenyl]acetamide

A mixture of 4-(1-cyclopropanecarbonyl-2,3-dihydro-1H-indol-5-yl)-5-methyl-1,3-thiazol-2-amine (0.08 g, 0.262 mmol, 1.0 eq), 2-[3-(4-{[2-(2,6-dioxopiperidin-3-yl)-1,3-dioxo-2,3-dihydro-1H-isoindol-4-yl]amino}butoxy)phenyl]acetic acid (0.145 g, 0.288 mmol, 1.1 eq) and NMI (0.108 g, 1.309 mmol, 5.0 eq) in anh ACN (0.65 ml, 0.4 M) and DMF (0.65 ml, 0.4 M) was cooled to 0° C., and then TCFH (0.22 g, 0.786 mmol, 3.0 eq) was added portionwise, and the reaction was stirred for overnight at rt. UPLC analysis showed 90% conversion. The solvents were evaporated to dryness and obtained material was dissolved in DCM, aq NaHCO₃ was added and it was stirred for 30 minutes at rt. It was extracted 3× with DCM, dried over Na₂SO₄ and evaporated to dryness. Then it was purified by prep. HPLC to give 81 mg (40% yield) of the title compound as a yellow solid. LCMS: [C₄₁H₄₀N₆O₇S], desired mass=760.87, observed mass=761.55 [M+H]⁺, ¹H NMR (300 MHz, DMSO) δ 12.36 (s, 1H), 11.09 (s, 1H), 8.07 (d, J=8.5 Hz, 1H), 7.57 (dd, J=8.6, 7.1 Hz, 1H), 7.50 (s, 1H), 7.42 (d, J=8.9 Hz, 1H), 7.23 (t, J=7.8 Hz, 1H), 7.12 (d, J=8.6 Hz, 1H), 7.01 (d, J=7.1 Hz, 1H), 6.94-6.80 (m, 3H), 6.63 (t, J=5.8 Hz, 1H), 5.05 (dd, J=12.8, 5.4 Hz, 1H), 4.39-4.28 (m, 2H), 4.01 (t, J=6.0 Hz, 2H), 3.70 (s, 2H), 3.43-3.36 (m, 2H), 3.23 (t, J=8.5 Hz, 2H), 2.94-2.81 (m, 1H), 2.63-2.54 (m, 2H), 2.44 (s, 3H), 2.07-1.94 (m, 2H), 1.83-1.70 (m, 0.95-0.81 (in, 4H).

### Example 55

### Synthesis of N-[4-(1-cyclopropanecarbonyl-2,3-dihydro-1H-indol-5-yl)-5-methyl-1,3-thiazol-2-yl]-2-[3-(4-{[2-(2,6-dioxopiperidin-3-yl)-1,3-dioxo-2,3-dihydro-1H-isoindol-4-yl]amino}butoxy)phenyl]acetamide (Cpd. No. 66)

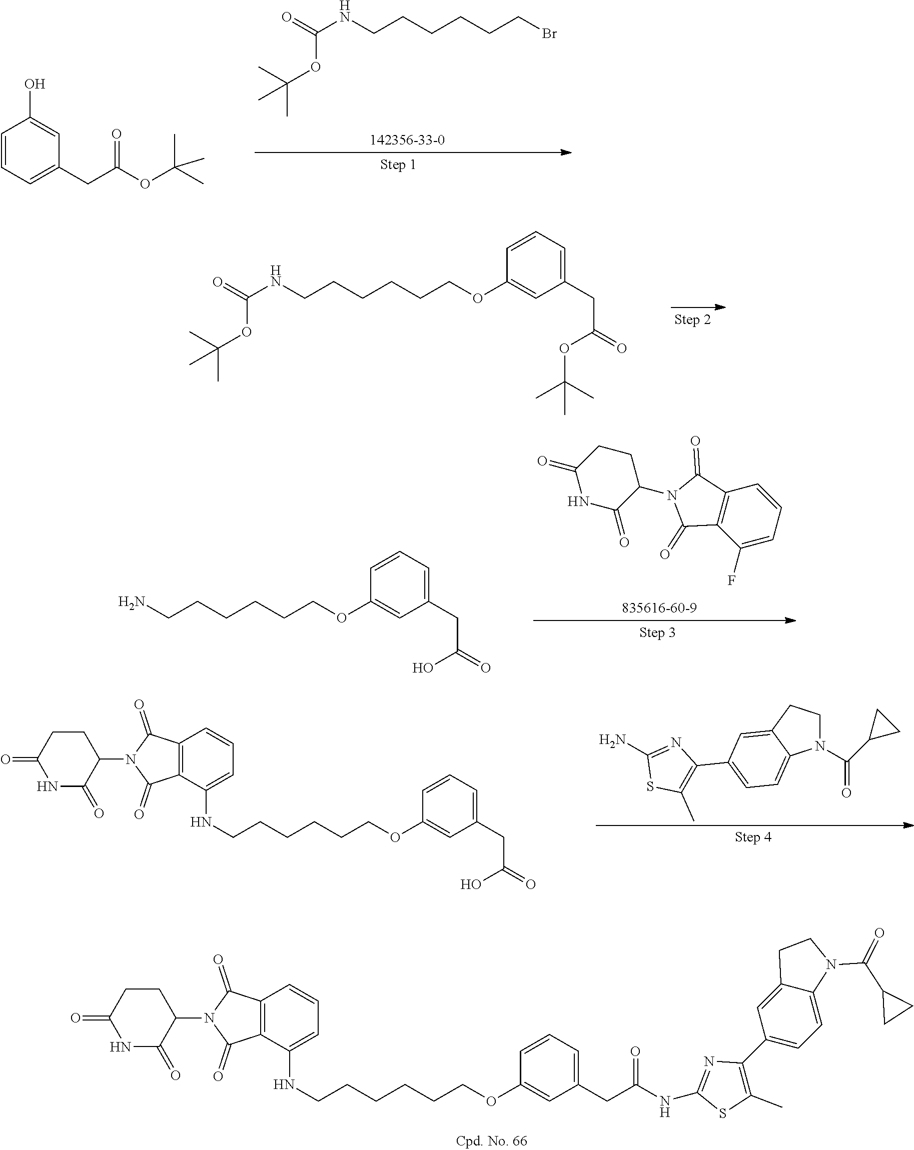

### Step 1: tert-butyl 2-{3-[(6-{[(tert-butoxy)carbonyl]amino}hexyl)oxy]phenyl}acetate

A mixture of tert-butyl 2-(3-hydroxyphenyl)acetate (0.5 g, 2.281 mmol, 1.0 eq), N-Boc-6-bromo-hexylamine (0.639 g, 2.281 mmol, 1.0 eq) and cesium carbonate (2.229 g, 6.843 mmol, 3.0 eq) in DMF (16.3 ml, 0.14 M) was stirred at room temperature overnight. RM was evaporated and to the residue was added EtOAc. The mixture was filtered and the filter cake was washed thoroughly with EtOAc. The filtrate was evaporated to give 0.959 g (quant. yield) of the title compound as a brown oil. LCMS: [C₂₃H₃₇NO₅], desired mass=407.551, observed mass=408.25 [M+H]⁺, ¹H NMR (300 MHz, Chloroform-d) δ 7.23 (t, J=7.8 Hz, 1H), 6.88-6.78 (m, 3H), 4.53 (s, 1H), 3.96 (t, J=6.4 Hz, 2H), 3.51 (s, 2H), 3.15 (q, J=6.5 Hz, 2H), 1.80 (p, J=6.6 Hz, 2H), 1.57-1.36 (m, 24H).

### Step 2: 2-{3-[(6-aminohexyl)oxy]phenyl}acetic acid; trifluoroacetic acid

Tert-butyl 2-{3-[(6-{[(tert-butoxy)carbonyl]amino}hexyl)oxy]phenyl}acetate (0.959 g, 2.282 mmol, 1.0 eq) was dissolved in a mixture of DCM (5.71 ml, 0.4 M) and TFA (7.808 g, 68.475 mmol, 30.0 eq), and it was stirred at rt for 1 h. UPLC analysis showed full conversion. It was evaporated on rotavap, dissolved a few times in DCM and then in ACN, followed by evaporation after every addition of the solvent and evaporated to dryness to get rid of remaining water. It was taken to the next step without purification. 1.09 g of the crude material as viscous oil with quantitative yield was obtained. LCMS: [C₁₆H₂₂F₃NO₅], desired mass=365.349, observed mass=252.35 [M+H]⁺, Remarks LCMS: rm 1 h ¹H NMR (300 MHz, DMSO) δ 7.82-7.50 (m, 3H), 7.22 (dd, J=9.2, 7.1 Hz, 1H), 6.87-6.61 (m, 3H), 4.74-4.66 (m, 1H), 3.95 (t, J=6.4 Hz, 2H), 3.53 (s, 2H), 2.87-2.73 (m, 2H), 1.78-1.66 (m, 2H), 1.61-1.50 (m, 2H), 1.49-1.31 (m, 4H).

### Step 3: 2-{3-[(6-{[2-(2,6-dioxopiperidin-3-yl)-1,3-dioxo-2,3-dihydro-1H-isoindol-4-yl]amino}hexyl)oxy]phenyl}acetic acid

A mixture of 2-(2,6-Dioxo-piperidin-3-yl)-4-fluoroisoindoline-1,3-dione (1.315 g, 4.762 mmol, 2.1 eq), 2-{3-[(6-aminohexyl)oxy]phenyl}acetic acid; trifluoroacetic acid (1.09 g, 2.267 mmol, 1.0 eq) and DIPEA (2.054 ml, 11.791 mmol, 5.2 eq) in NMP (7.56 ml, 0.3 M) was stirred at 100° C. for overnight. UPLC analysis showed full conversion. DIPEA was evaporated, and the solution in NMP was injected on the column for RPFC. It was purified in acidic conditions with 0.1% FA and eluted with ACN:H₂O (10-59% of ACN) to give 640 mg (55% yield) of the title compound as a green solid. LCMS: [C₂₇N₃O₇], desired mass=507.543, observed mass=508.3 [M+H]⁺, ¹H NMR (300 MHz, DMSO) δ 12.19 (s, 1H), 11.09 (s, 1H), 7.58 (dd, J=8.5, 7.1 Hz, 1H), 7.19 (dd, J=9.0, 6.9 Hz, 1H), 7.11 (d, J=8.6 Hz, 1H), 7.02 (d, J=7.0 Hz, 1H), 6.84-6.75 (m, 3H), 6.55 (t, J=6.0 Hz, 1H), 5.05 (dd, J=12.8, 5.4 Hz, 1H), 3.94 (t, J=6.4 Hz, 2H), 3.51 (s, 2H), 2.98-2.80 (m, 1H), 2.65-2.52 (m, 2.07-1.98 (m, 1H), 1.79-1.67 (m, 2H), 1.66-1.55 (m, 2H), 1.52-1.36 (m, 4H).

### Step 4: N-[4-(1-cyclopropanecarbonyl-2,3-dihydro-1H-indol-5-yl)-5-methyl-1,3-thiazol-2-yl]-2-{3-[(6-{[2-(2,6-dioxopiperidin-3-yl)-1,3-dioxo-2,3-dihydro-1H-isoindol-4-yl]amino}hexyl)oxy]phenyl}acetamide

A mixture of 4-(1-cyclopropanecarbonyl-2,3-dihydro-1H-indol-5-yl)-5-methyl-1,3-thiazol-2-amine (0.08 g, 0.262 mmol, 1.0 eq), 1-Methylimidazole (0.108 g, 1.309 mmol, 5.0 eq) and 2-{3-[(6-{[2-(2,6-dioxopiperidin-3-yl)-1,3-dioxo-2,3-dihydro-1H-isoindol-4-yl]amino}hexyl)oxy]phenyl}acetic acid (0.149 g, 0.288 mmol, 1.1 eq) in anh DMF (0.65 ml, 0.4 M) and anh ACN (0.65 ml, 0.4 M) was cooled to 0° C. and then TCFH (0.22 g, 0.786 mmol, 3.0 eq) was added and the RM was stirred at rt for overnight. UPLC analysis showed 90% conversion. The solvents were evaporated to dryness and obtained material was dissolved in DCM, aq NaHCO₃ was added and it was stirred for 30 minutes at rt. It was extracted 3× with DCM, dried over Na₂SO₄ and evaporated to dryness. Then it was purified by prep. HPLC to give 80 mg (38% yield) of the title compound as a yellow solid. LCMS: [C₄₃H₄₄N₆O₇S], desired mass=788.92, observed mass=789.25 [M+H]⁺, 1H NMR (400 MHz, DMSO) δ 12.29 (s, 1H), 11.10 (s, 1H), 8.07 (d, J=8.4 Hz, 1H), 7.57 (dd, J=8.6, 7.1 Hz, 1H), 7.50 (s, 1H), 7.43 (d, J=8.5 Hz, 1H), 7.23 (t, J=7.9 Hz, 1H), 7.09 (d, J=8.6 Hz, 1H), 7.02 (d, J=7.0 Hz, 1H), 6.94-6.87 (m, 2H), 6.85-6.79 (m, 1H), 6.55 (t, J=6.0 Hz, 1H), 5.05 (dd, J=12.8, 5.4 Hz, 1H), 4.41-4.25 (m, 2H), 3.96 (t, J=6.4 Hz, 2H), 3.70 (s, 2H), 3.32-3.27 (m, 2H), 3.22 (t, 2H), 2.88 (ddd, J=17.4, 14.0, 5.4 Hz, 1H), 2.63-2.53 (m, 2H), 2.44 (s, 3H), 2.07-1.93 (m, 2H), 1.72 (q, J=6.9 Hz, 2H), 1.61 (p, J=7.1 Hz, 2H), 1.53-1.36 (m, 0.96-0.80 (m, 4H).

### Example 56

### Synthesis of N-[4-(1-cyclopropanecarbonyl-2,3-dihydro-1H-indol-5-yl)-5-methyl-1,3-thiazol-2-yl]-2-[3-(2-{1-[2-(2,6-dioxopiperidin-3-yl)-1,3-dioxo-2,3-dihydro-1H-isoindol-5-yl]piperidin-4-yl}ethoxy)phenyl]acetamide (Cpd. No. 68)

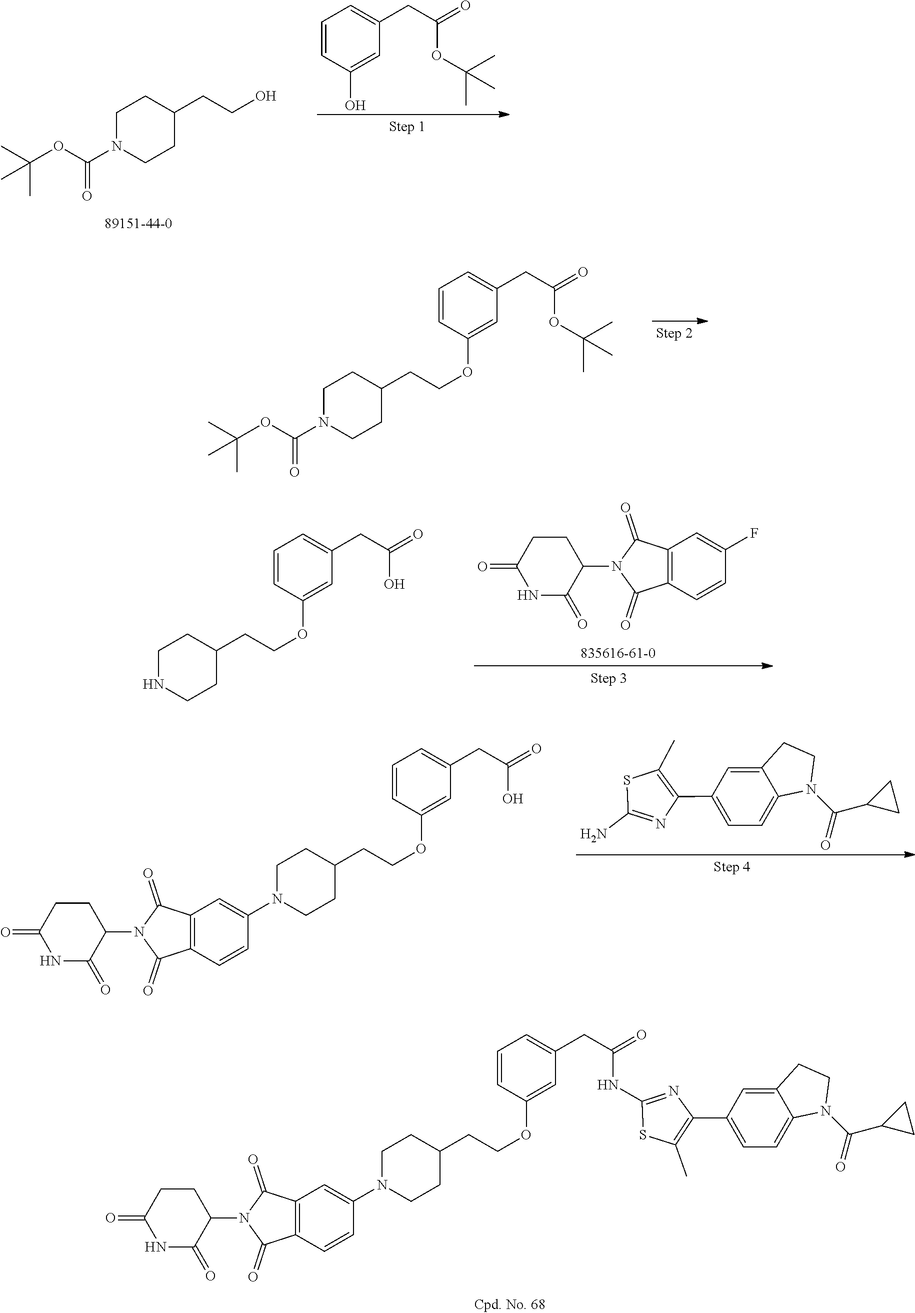

### Step 1: tert-butyl 4-(2-{3-[2-(tert-butoxy)-2-oxoethyl]phenoxy}ethyl)piperidine-1-carboxylate

To a stirred solution of 1-Boc-4-(2-hydroxyethyl)piperidine (0.628 g, 2.737 mmol, 1.2 eq), tert-butyl 2-(3-hydroxyphenyl)acetate (0.5 g, 2.281 mmol, 1.0 eq), and triphenylphosphine (0.897 g, 3.421 mmol, 1.5 eq) in tetrahydrofuran (22.81 ml, 0.1 M) was added diisopropyl azodicarboxylate; DIAD (0.692 g, 3.421 mmol, 1.5 eq) dropwise at 0° C. and the RM was stirred at rt on. Diethyl ether was added to the RM and it was filtered. Solid was washed with Et₂O and solvents was removed. Residue was purified by FC (EtOAc/c-Hexane 0:100 to 30:70) to give tert-butyl 4-(2-{3-[2-(tert-butoxy)-2-oxoethyl]phenoxy}ethyl)piperidine-1-carboxylate (0.683 g, 1.628 mmol, 71%) LCMS: [C₂₄H₃₇NO₅], desired mass=419.56, observed mass=420.20 [M+H]⁺ ¹H NMR (300 MHz, Chloroform-d) δ 7.21 (t, J=7.8 Hz, 1H), 6.87-6.74 (m, 3H), 4.09 (d, J=12.1 Hz, 2H), 4.00 (t, J=5.9 Hz, 2H), 3.49 (s, 2H), 2.70 (t, J=12.8 Hz, 2H), 1.77-1.64 (m, 5H), 1.46 (s, 9H), 1.44 (s, 9H), 1.24-1.09 (m, 2H).

### Step 2: 2-{3-[2-(piperidin-4-yl)ethoxy]phenyl}acetic acid; trifluoroacetic acid

To a solution of tert-butyl 4-(2-{3-[2-(tert-butoxy)-2-oxoethyl]phenoxy}ethyl)piperidine-1-carboxylate (0.683 g, 1.628 mmol, 1.0 eq) in dichloromethane (5.43 ml, 0.3 M) was added trifluoroacetic acid (5.569 g, 48.837 mmol, 30.0 eq) and stirred at room temperature for 2 h. Solvent was removed in vacuo to give 2-{3-[2-(piperidin-4-yl)ethoxy]phenyl}acetic acid; trifluoroacetic acid (0.691 g, 1.621 mmol, 100%) as a brown oil. Crude was taken to the next step. LCMS: [C₁₅H₂₁NO₃], desired mass=263.34, observed mass=264.40 [M+H], ¹H NMR (300 MHz, DMSO-d₆) δ 12.28 (s, 1H), 8.53 (s, 1H), 8.22 (s, 1H), 7.25-7.17 (m, 1H), 6.81 (dt, J=5.6, 1.9 Hz, 3H), 3.99 (t, J=6.1 Hz, 2H), 3.52 (s, 2H), 3.31-3.21 (m, 2H), 2.95-2.79 (m, 2H), 1.91-1.63 (m, 5H), 1.41-1.21 (m, 21-1).

### Step 3: 2-[3-(2-{1-[2-(2,6-dioxopiperidin-3-yl)-1,3-dioxo-2,3-dihydro-1H-isoindol-5-yl]piperidin-4-yl}ethoxy)phenyl]acetic acid

To a stirred solution of 2-(2,6-dioxopiperidin-3-yl)-5-fluoroisoindoline-1,3-dione (0.292 g, 1.055 mmol, 1.5 eq) and: 2-{3-[2-(piperidin-4-yl)ethoxy]phenyl}acetic acid; trifluoroacetic acid (0.3 g, 0.704 mmol, 1.0 eq) in dimethyl sulfoxide (1.56 ml, 0.45 M) was added N,N-diisopropylethylamine (DIPEA) (0.112 ml, 0.645 mmol, 5.5 eq) and the RM was stirred at 100° C. in a sealed tube on. UPLC showed full conversion to DP and the RM without work-up loaded to RPFC column to give 2-[3-(2-{1-[2-(2,6-dioxopiperidin-3-yl)-1,3-dioxo-2,3-dihydro-1H-isoindol-5-yl]piperidin-4-yl}ethoxy)phenyl]acetic acid (0.469 g, 0.555 mmol, 77% yield) as a yellow solid with 460% purity in a mixture with starting fluoride and DIPEA salt. LCMS: [C₂₈H₂₉N₃O₇], desired mass=519.55, observed mass=520.30 [M+H], ¹H NMR (300 MHz, DMSO-d₆) δ 11.08 (s, 1H), 7.65 (d, J=8.5 Hz, 1H), 7.31 (d, J=2.3 Hz, 1H), 7.27-7.16 (m, 2H), 6.85-6.78 (m, 3H), 5.06 (dd, J=12.6, 5.4 Hz, 1H), 4.11-3.96 (m, 3H), 3.51 (s, 2H), 3.06-2.81 (m, 3H), 2.67-2.41 (m, 31H), 2.12-1.95 (m, 2H), 1.87-1.74 (m, 2H), 1.73-1.64 (m, 2H), 1.35-1.17 (m, 2H).

### Step 4: N-[4-(1-cyclopropanecarbonyl-2,3-dihydro-1H-indol-5-yl)-5-methyl-1,3-thiazol-2-yl]-2-[3-(2-{1-[2-(2,6-dioxopiperidin-3-yl)-1,3-dioxo-2,3-dihydro-1H-isoindol-5-yl]piperidin-4-yl}ethoxy)phenyl]acetamide

To a mixture of (5-(2-amino-5-methylthiazol-4-yl)indolin-1-yl)(cyclopropyl)methanone (0.035 g, 0.115 mmol, 1.0 eq), 2-[3-(2-{1-[2-(2,6-dioxopiperidin-3-yl)-1,3-dioxo-2,3-dihydro-1H-isoindol-5-yl]piperidin-4-yl}ethoxy)phenyl]acetic acid and 1-methylimidazole (0.057 g, 0.693 mmol, 6.0 eq) in acetonitrile (0.58 ml, 0.2 M) and dimethylfornamide (0.58 ml, 0.2 M) was added N,N,N′,N′-tetramethylchloroformamidinium hexafluorophosphate (0.097 g, 0.346 mmol, 3.0 eq) and the RM was stirred at rt overnight. Then it was evaporated to dryness, dissolved in DCM and saturated NaHCO₃ was added. It was stirred for 15 minutes and then extracted with DCM, dried over sodium sulfate, filtered and evaporated to dryness. Residue was purified by prepHPLC to give N-[4-(1-cyclopropanecarbonyl-2,3-dihydro-1H-indol-5-yl)-5-methyl-1,3-thiazol-2-yl]-2-[3-(2-{1-[2-(2,6-dioxopiperidin-3-yl)-1,3-dioxo-2,3-dihydro-1H-isoindol-5-yl]piperidin-4-yl}ethoxy)phenyl]acetamide (0.027 g, 0.034 mmol, 29% yield) as a yellow solid. LCMS: [C₄₄H₄₄N₆O₇S], desired mass=800.93, observed mass=801.09 [M+H]⁺, ¹H NMR (300 MHz, DMSO-d₆) δ 12.29 (s, 1H), 11.07 (s, 1H), 8.05 (d, J=8.4 Hz, 1H), 7.63 (d, J=8.5 Hz, 1H), 7.49 (s, 1H), 7.41 (dd, J=8.4, 1.9 Hz, 1H), 7.31-7.17 (m, 3H), 6.95-6.80 (m, 3H), 5.05 (dd, J=12.7, 5.3 Hz, 1H), 4.31 (t, J=8.5 Hz, 2H), 4.08-3.97 (m, 3.70 (s, 2H), 3.21 (t, J=8.5 Hz, 2H), 3.02-2.80 (m, 3H), 2.64-2.53 (m, 2H), 2.42 (s, 3H), 2.07-1.89 (m, 2H), 1.85-1.74 (m, 3H), 1.67 (q, J=6.3 Hz, 2H), 1.32-1.15 (m, 3H), 0.93-0.81 (m, 4H).

### Example 57

### Synthesis of N-[4-(1-cyclopropanecarbonyl-2,3-dihydro-1H-indol-5-yl)-5-methyl-1,3-thiazol-2-yl]-2-{3-[(5-{[2-(2,6-dioxopiperidin-3-yl)-1-oxo-2,3-dihydro-1H-isoindol-4-yl]amino}pentyl)oxy]phenyl}acetamide (Cpd. No. 69)

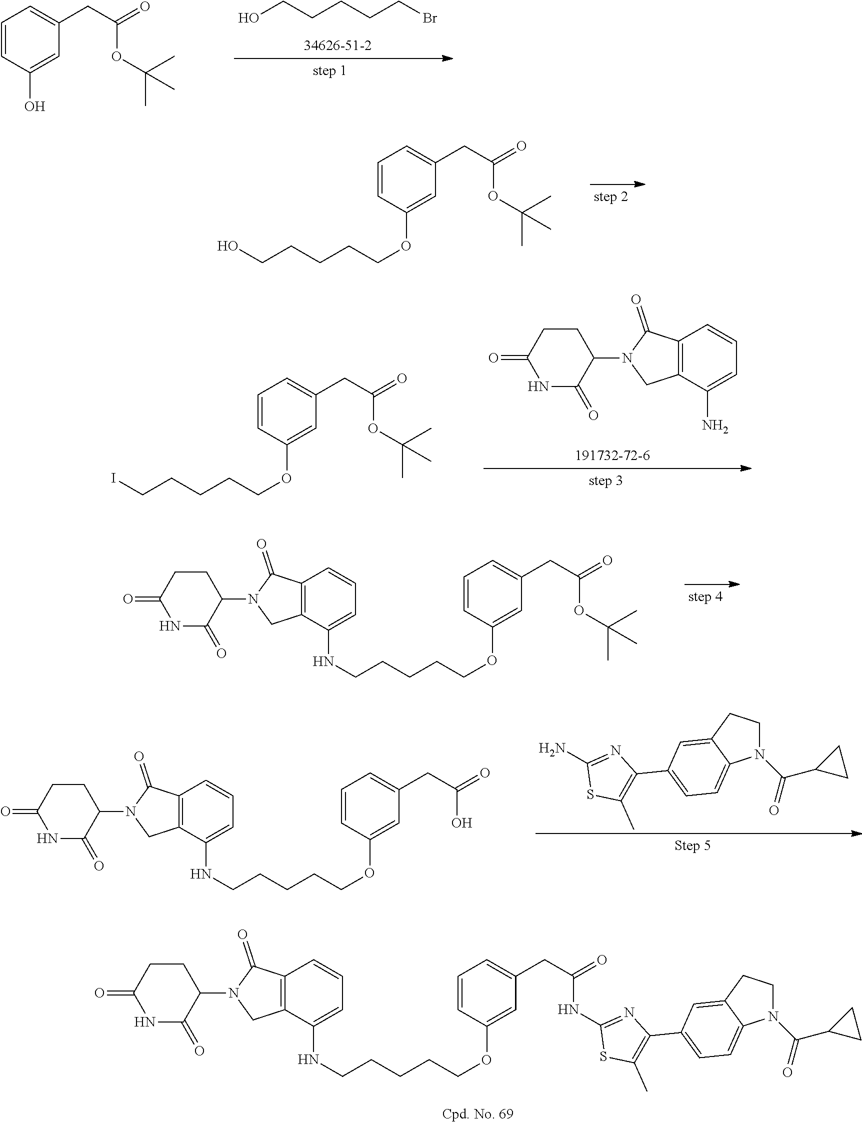

### Step 1: tert-butyl 2-{3-[(5-hydroxypentyl)oxy]phenyl}acetate

To a stirred solution of 5-bromo-1-pentanol (2.2 eq) and tert-butyl 2-(3-hydroxyphenyl)acetate (0.5 g, 2.401 mmol, 1.0 eq) in dimethylformamide (4.8 ml, 0.5 M) was added potassium carbonate anhydrous (2.0 eq) and the mixture was stirred at 75° C. o/n. UPLC showed ˜70% conversion. 2-(2-bromoethoxy)ethanol (1 eq) and potassium carbonate anhydrous (0.5 eq) was added and the RM was stirred at 75° C. o/n to show full conversion. The RM was diluted with Et₂O and filtered. Solid was washed with EtOAc and filtrate was evaporated, residue was purified by FC EtOAc/C-Hex (0:100 to 30:70) to give tert-butyl 2-{3-[(5-hydroxypentyl)oxy]phenyl}acetate (0.189 g, 0.61 mmol, 25%). The yield was low due to low purity of starting commercial bromide. ¹H NMR (300 MHz, Chlorofom-d) δ 7.21 (t, J=7.7 Hz, 1H), 6.86-6.75 (m, 3H), 3.96 (t, J=6.4 Hz, 2H), 3.68 (t, J=6.3 Hz, 2H), 3.48 (s, 2H), 1.81 (p, J=6.7 Hz, 2H), 1.71-1.48 (m, 1.44 (s, 9H), 1.34 (s, 1H).

### Step 2: tert-butyl 2-{3-[(5-iodopentyl)oxy]phenyl}acetate

To a stirred solution of tert-butyl 2-{3-[(5-hydroxypentyl)oxy]phenyl}acetate (0.189 g, 0.61 mmol, 1.0 eq), imidazole (0.062 g, 0.915 mmol 1.5 eq) and triphenylphosphine (0.24 g, 0.915 mmol, 1.5 eq) in dichloromethane (4.07 ml, 0.15 M) was added iodine (0.232 g, 0.915 mmol, 1.5 eq) at 0° C. The RM was stirred at rt o/n to show full conversion. Et₂O was added to the RM and it was filtered and solid was washed with Et₂O. Solvents were evaporated in vacuo and the residue was purified by FC: EtOAc/C-Hex (0:100 to 15:85) to give tert-butyl 2-{3-[(5-iodopentyl)oxy]phenyl}acetate (0.227 g, 0.561 mmol, 92%) as a clear light-yellow oil. ¹H NMR (300 MHz, Chloroform-d) δ 7.21 (t, J=7.8 Hz, 1H), 6.87-6.75 (m, 3H), 3.96 (t, J=6.3 Hz, 2H), 3.49 (s, 2H), 3.22 (t, J=7.0 Hz, 2H), 1.90 (dt, J=14.8, 7.1 Hz, 2H), 1.79 (dt, J=8.4, 6.4 Hz, 2H), 1.66-1.52 (m, 2H), 1.44 (s, 9H).

### Step 3: tert-butyl 2-{3-[(5-{[2-(2,6-dioxopiperidin-3-yl)-1-oxo-2,3-dihydro-1H-isoindol-4-yl]amino}pentyl)oxy]phenyl}acetate

A solution of lenalidomide (0.146 g, 0.561 mmol, 1.0 eq), N,N-diisopropylethylamine (DIPEA) (0.293 ml, 1.684 mmol, 3.0 eq) and tert-butyl 2-{3-[(5-iodopentyl)oxy]phenyl}acetate (0.227 g, 0.561 mmol, 1.0 eq) in N-methyl-2-pyrrolidone (2.81 ml, 0.2 M) was stirred at 80° C. in a sealed vial o/n. EtOAc and water was added to the RM and water phase was extracted with EtOAc. Combined organic phases was dried over Na₂SO₄, solvent was evaporated in vacuo and residue was purified by FC (DCM/MeOH, 100:0 to 95:5) to give tert-butyl 2-{3-[(5-{[2-(2,6-dioxopiperidin-3-yl)-1-oxo-2,3-dihydro-1H1-isoindol-4-yl]amino}pentyl)oxy]phenyl}acetate (0.163 g, 0.304 mmol, 54%). LCMS: [C₃₀H₃₇N₃O₆], desired mass=535.64, observed mass=536.30 [M+H]⁺, ¹H NMR (300 MHz, DMSO-d₆) δ 11.00 (s, 1H), 7.28 (t, J=7.7 Hz, 1H), 7.26-7.14 (m, 1H), 6.92 (d, J=7.4 Hz, 1H), 6.83-6.72 (m, 5.58 (t, J=5.6 Hz, 1H), 5.11 (dd, J=13.2, 5.1 Hz, 1H), 4.23 (d, J=17.2 Hz, 1H), 4.12 (d, J=17.2 Hz, 1H), 3.96 (t, J=6.4 Hz, 2H), 3.50 (s, 2H), 3.15 (q, J=6.6 Hz, 2H), 3.00-2.85 (m, 1H), 2.67-2.56 (m, 1H), 2.29 (qd, J=13.5, 4.5 Hz, 1H), 2.09-1.96 (m, 1H), 1.82-1.71 (m, 2H), 1.70-1.59 (m, 2H), 1.58-1.46 (m, 2H), 1.39 (s, 9H).

### Step 4: 2-{3-[(5-{[2-(2,6-dioxopiperidin-3-yl)-1-oxo-2,3-dihydro-1H-isoindol-4-yl]amino}pentyl)oxy]phenyl}acetic acid; trifluoroacetic acid

To a solution of tert-butyl 2-{3-[(5-{[2-(2,6-dioxopiperidin-3-yl)-1-oxo-2,3-dihydro-1H-isoindol-4-yl]amino}pentyl)oxy]phenyl}acetate (0.08 g, 0.149 mmol, 1.0 eq) in dichloromethane (0.5 ml, 0.3 M) was added trifluoroacetic acid (0.511 g, 4.481 mmol, 30.0 eq) and the RM stirred at room temperature for 2 h. The solvent was removed in vacuo to give 2-{3-[(5-{[2-(2,6-dioxopiperidin-3-yl)-1-oxo-2,3-dihydro-1H-isoindol-4-yl]amino}pentyl)oxy]phenyl}acetic acid; trifluoroacetic acid (0.089 g, 0.15 mmol, 100%) as a yellow oil. Crude was taken to the next step. LCMS: [C₂₆H₂₉N₃O₆], desired mass=479.53, observed mass=480.60 [M+H]⁺.

### Step 5: N-[4-(1-cyclopropanecarbonyl-2,3-dihydro-1H-indol-5-yl)-5-methyl-1,3-thiazol-2-yl]-2-{3-[(5-{[2-(2,6-dioxopiperidin-3-yl)-1-oxo-2,3-dihydro-1H-isoindol-4-yl]amino}pentyl)oxy]phenyl}acetamide

To a mixture of 4-(1-cyclopropanecarbonyl-2,3-dihydro-1H-indol-5-yl)-5-methyl-1,3-thiazol-2-amine (0.046 g, 0.149 mmol, 1.0 eq), 2-{3-[(5-{[2-(2,6-dioxopiperidin-3-yl)-1-oxo-2,3-dihydro-1H-isoindol-4-yl]amino}pentyl)oxy]phenyl}acetic acid; trifluoroacetic acid (0.072 g, 0.149 mmol, 1.0 eq) and 1-methylimidazole (0.074 g, 0.896 mmol, 6.0 eq) in acetonitrile (0.75 ml, 0.2 M) and dimethylformamide (0.75 ml, 0.2 M) was added N,N,N′,N′-tetramethylchloroformamidinium hexafluorophosphate (0.126 g, 0.448 mmol, 3.0 eq) and the RM was stirred at rt overnight. Then it was evaporated to dryness, dissolved in DCM and saturated NaHCO₃ and water was added. It was stirred for 30 minutes and then extracted with DC M, dried over sodium sulfate, filtered and evaporated to dryness. Residue was purified by prepHPLC to give N-[4-(1-cyclopropanecarbonyl-2,3-dihydro-1H-indol-5-yl)-5-methyl-1,3-thiazol-2-yl]-2-{3-[(5-{[2-(2,6-dioxopiperidin-3-yl)-1-oxo-2,3-dihydro-1H-isoindol-4-yl]amino}pentyl)oxy]phenyl}acetamide (0.038 g, 0.049 mmol, 32%) as a white solid. LCMS: [C₄₂H₄₄N₆O₆S], desired mass=760.91, observed mass=761.8 [M+H]⁺, 759.4 [M−H]⁻, ¹H NMR (300 MHz, DMSO-d₆) δ 12.28 (s, 1H), 11.00 (s, 1H), 8.06 (d, J=8.1 Hz, 1H), 7.49 (s, 1H), 7.42 (d, J=8.5 Hz, 1H), 7.31-7.19 (m, 2H), 6.95-6.79 (m, 6.75 (d, J=8.1 Hz, 1H), 5.58 (t, J=5.5 Hz, 1H), 5.10 (dd, J=13.2, 5.1 Hz, 1H), 4.37-4.27 (m, 2H), 4.23 (d, J=17.2 Hz, 1H), 4.12 (d, J=17.2 Hz, 1H), 3.96 (t, J=6.3 Hz, 2H), 3.69 (s, 2H), 3.27-3.10 (m, 2.99-2.84 (m, 1H), 2.66-2.56 (m, 1H), 2.43 (s, 3H), 2.37-2.21 (m, 1H), 2.08-1.91 (m, 2H), 1.82-1.71 (m, 2H), 1.70-1.59 (m, 2H), 1.58-1.46 (m, 2H), 0.91-0.82 (m, 4H).

### Example 58

### Synthesis of N-[4-(1-cyclopropanecarbonyl-2,3-dihydro-1H-indol-5-yl)-5-methyl-1,3-thiazol-2-yl]-2-{3-[2-(2-{[2-(2,6-dioxopiperidin-3-yl)-3-oxo-2,3-dihydro-1H-isoindol-4-yl]amino}ethoxy)ethoxy]phenyl}acetamide (Cpd. No. 81)

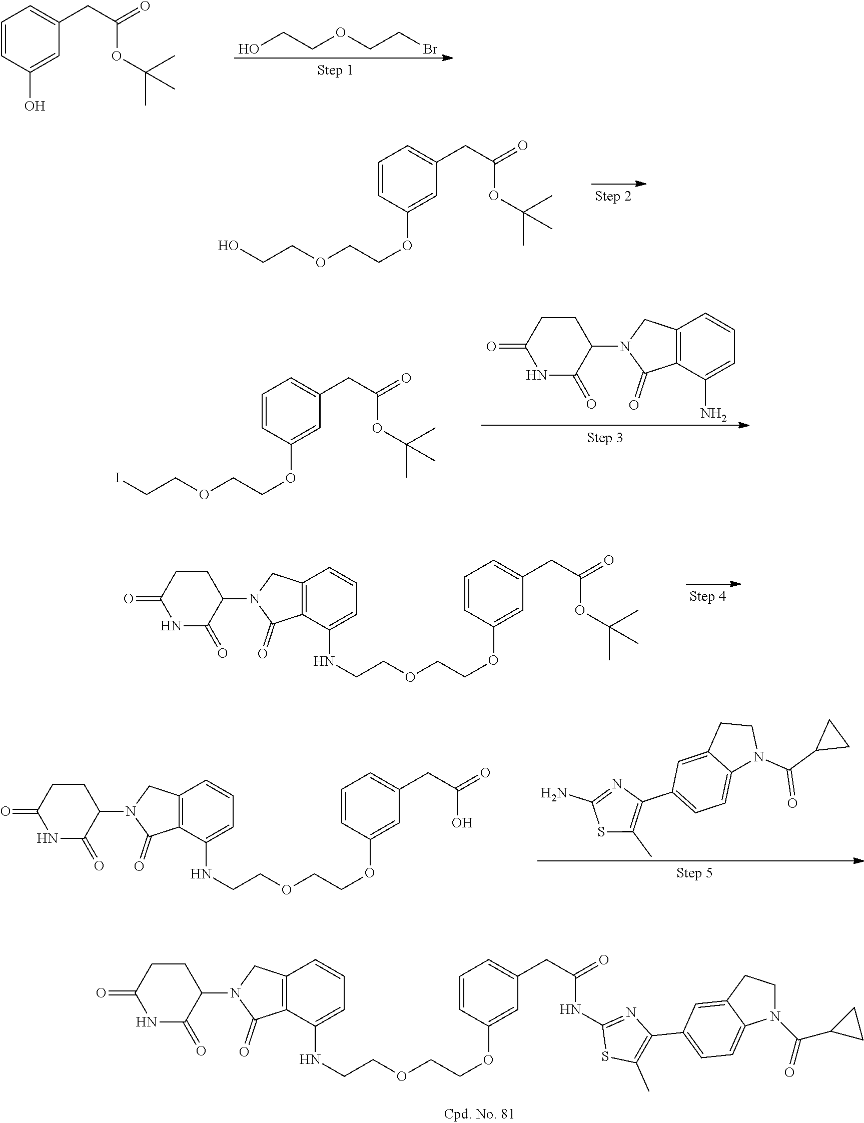

### Step 1: tert-butyl 2-{3-[2-(2-hydroxyethoxy)ethoxy]phenyl}acetate

To a stirred solution of 2-(2-bromoethoxy)ethanol (1.25 g, 7.395 mmol, 2.2 eq) and tert-butyl 2-(3-hydroxyphenyl)acetate (0.7 g, 3.361 mmol, 1.0 eq) in dimethylformamide (6.72 ml, 0.5 M) was added potassium carbonate (0.929 g, 6.722 mmol, 2.0 eq) and the mixture was stirred at 75° C. o/n. The RM was diluted with Et₂O and filtered. Solid was washed with EtOAc and filtrate was evaporated. Residue was purified by FC (EtOAc/c-Hex: 0:100 to 50:50) to give tert-butyl 2-{3-[2-(2-hydroxyethoxy)ethoxy]phenyl}acetate (1.03 g, 3.302 mmol, 98%). ¹H NMR (300 MHz, Chloroform-d) δ 7.22 (t, J=8.1 Hz, 1H), 6.89-6.78 (m, 3H), 4.16-4.11 (m, 2H), 3.89-3.83 (m, 2H), 3.79-3.72 (m, 2H), 3.69-3.65 (m, 2H), 3.49 (s, 2H), 2.23 (t, J=6.2 Hz, 1H), 1.43 (s, 91H).

### Step 2: tert-butyl 2-{3-[2-(2-iodoethoxy)ethoxy]phenyl}acetate

To a stirred solution of tert-butyl 2-{3-[2-(2-hydroxyethoxy)ethoxy]phenyl}acetate (1.03 g, 3.302 mmol), imidazole (1.5 eq) and triphenylphosphine (1.5 eq) in dichloromethane (22 ml, 0.15 M) was added iodine (1.5 eq) in portions at 0° C. The RM was stirred at rt o/n to show full conversion. Eₜ₂O was added to the RM and it was filtered and solid was washed with Et₂O. Solvents were evaporated in vacuo and residue was purified by FC: EtOAc/C-Hex (0:100 to 15:85) to give tert-butyl 2-{3-[2-(2-iodoethoxy)ethoxy]phenyl}acetate (0.99 g, 2.437 mmol, 75% yield) as a clear light-yellow oil. ¹H NMR (300 MHz, Chloroform-d) δ 7.22 (t, J=8.1 Hz, 1H), 6.89-6.78 (m, 3H), 4.16-4.10 (m, 2H), 3.90-3.79 (m, 3.49 (s, 2H), 3.32-3.24 (m, 2H), 1.44 (s, 8H).

### Step 3: tert-butyl 2-{3-[2-(2-{[2-(2,6-dioxopiperidin-3-yl)-3-oxo-2,3-dihydro-1H-isoindol-4-yl]amino}ethoxy)ethoxy]phenyl}acetate

A solution of 3-(7-amino-1,3-dihydro-1-oxo-2H-isoindol-2-yl)-2,6-piperidinedione (0.097 g, 0.374 mmol, 1.0 eq), N,N-diisopropylethylamine (DIPEA) (0.163 ml, 2.5 eq) and tert-butyl 2-{3-[2-(2-iodoethoxy)ethoxy]phenyl}acetate (0.152 g, 1.0 eq) in N-methyl-2-pyrrolidone (1.87 ml, 0.2 M) was stirred at 80° C. in a sealed vial o/n to show ˜15% conversion. The RM was stirred at 100-110° C. for 4 d to show ˜40% conversion. N,N-diisopropylethylamine (DIPEA) (0.163 ml, 2.5 eq) and tert-butyl 2-{3-[2-(2-iodoethoxy)ethoxy]phenyl}acetate (0.152 g, 1.0 eq) was added to the RM and the mixture was stirred at 110° C. o/n. EtOAc and water was added to the RM and water phase was extracted with EtOAc. Combined organic phases was dried over Na₂SO₄, solvent was evaporated in vacuo and residue was purified by FC (DCM/MeOH, 100:0 to 95:5) and then RPFC to give tert-butyl 2-{3-[2-(2-{[2-(2,6-dioxopiperidin-3-yl)-3-oxo-2,3-dihydro-1H-isoindol-4-yl]amino}ethoxy)ethoxy]phenyl}acetate (0.098 g, 0.182 mmol, 49%). LCMS: [C₂₉H₃₅N₃O₇], desired mass=537.61, observed mass=538.20 [M+H], 11H NMR (300 MHz, Chloroform-d) δ 7.92 (s, 1H), 7.33 (t, J=7.8 Hz, 1H), 7.20 (t, J=7.9 Hz, 1H), 6.90-6.74 (m, 6.63 (d, J=7.4 Hz, 1H), 6.59 (d, J=8.3 Hz, 1H), 5.08 (dd, J=13.2, 5.2 Hz, 1H), 4.37 (d, J=16.0 Hz, 1H), 4.24 (d, J=16.0 Hz, 1H), 4.14 (t, J=4.9 Hz, 2H), 3.86 (t, J=4.9 Hz, 2H), 3.79 (t, J=5.8 Hz, 2H), 3.50-3.43 (m, 2H), 3.48 (s, 2H), 2.96-2.73 (m, 2H), 2.33 (qd, J=13.0, 5.2 Hz, 1H), 2.23-2.10 (m, 1H), 1.43 (s, 9H).

### Step 4: 2-{3-[2-(2-{[2-(2,6-dioxopiperidin-3-yl)-3-oxo-2,3-dihydro-1H-isoindol-4-yl]amino}ethoxy)ethoxy]phenyl}acetic acid; trifluoroacetic acid

To a solution of tert-butyl 2-{3-[2-(2-{[2-(2,6-dioxopiperidin-3-yl)-3-oxo-2,3-dihydro-1H-isoindol-4-yl]amino}ethoxy)ethoxy]phenyl}acetate (0.098 g, 0.182 mmol, 1.0 eq) in dichloromethane (0.61 ml, 0.3 M) was added trifluoroacetic acid (0.622 g, 5.452 mmol, 30.0 eq) and the RM stirred at room temperature for 1 h. The solvent was removed in vacuo to give 2-{3-[2-(2-{[2-(2,6-dioxopiperidin-3-yl)-3-oxo-2,3-dihydro-1H-isoindol-4-yl]amino}ethoxy)ethoxy]phenyl}acetic acid; trifluoroacetic acid (0.108 g, 0.172 mmol, 95%) as a brown oil. Crude was taken to the next step. LCMS: [C₂₅H₂₇N₃O₇], desired mass=481.51, observed mass=482.50 [M+H]⁺.

### Step 5: N-[4-(1-cyclopropanecarbonyl-2,3-dihydro-1H-indol-5-yl)-5-methyl-1,3-thiazol-2-yl]-2-{3-[2-(2-{[2-(2,6-dioxopiperidin-3-yl)-3-oxo-2,3-dihydro-1H-isoindol-4-yl]amino}ethoxy)ethoxy]phenyl}acetamide

To a mixture of (5-(2-amino-5-methylthiazol-4-yl)indolin-1-yl)(cyclopropyl)methanone 0.055 g, 0.181 mmol, 1.05 eq), 2-{3-[2-(2-{[2-(2,6-dioxopiperidin-3-yl)-3-oxo-2,3-dihydro-1H-isoindol-4-yl]amino}ethoxy)ethoxy]phenyl}acetic acid; trifluoroacetic acid (0.108 g, 0.173 mmol, 1.0 eq) and 1-methylimidazole (0.085 g, 1.036 mmol, 6.0 eq) in acetonitrile (0.86 ml, 0.2 M) and dimethylformamide (0.86 ml, 0.2 M) was added N,N,N′,N′-tetramethylchloroformamidinium hexafluorophosphate (0.145 g, 0.518 mmol, 3.0 eq) and the RM was stirred at rt overnight. Then it was evaporated to dryness, dissolved in DCM and saturated NaHCO₃ was added. It was stirred for 15 minutes and then extracted with DCM, dried over sodium sulfate, filtered and evaporated to dryness. Residue was purified by prepHPLC to give N-[4-(1-cyclopropanecarbonyl-2,3-dihydro-1H-indol-5-yl)-5-methyl-1,3-thiazol-2-yl]-2-{3-[2-(2-{[2-(2,6-dioxopiperidin-3-yl)-3-oxo-2,3-dihydro-1H-isoindol-4-yl]amino}ethoxy)ethoxy]phenyl}acetamide (0.031 g, 23%) as a light-yellow solid. LCMS: [C₄₁H₄₂N₆O₇S], desired mass=762.88, observed mass=763.35 [M+H]⁺, 761.45 [M−H]⁻, ¹H NMR (300 MHz, DMSO-d₆) δ 12.27 (s, 1H), 10.96 (s, 1H), 8.06 (d, J=8.4 Hz, 1H), 7.49 (s, 1H), 7.42 (d, J=7.7 Hz, 1H), 7.32 (t, J=7.8 Hz, 1H), 7.22 (t, J=7.9 Hz, 1H), 6.95-6.80 (m, 3H), 6.70-6.60 (m, 2H), 4.99 (dd, J=13.2, 5.1 Hz, 1H), 4.38-4.24 (m, 3H), 4.18 (d, J=17.3 Hz, 1H), 4.13-4.05 (m, 2H), 3.78 (t, J=4.6 Hz, 2H), 3.73-3.64 (m, 3.22 (t, J=8.5 Hz, 2H), 2.88 (ddd, J=18.1, 13.5, 5.4 Hz, 1H), 2.64-2.53 (m, 1H), 2.43 (s, 3H), 2.32 (qd, J=13.1, 4.4 Hz, 1H), 2.01-1.89 (m, 2H), 0.92-0.81 (m, 4H).

### Example 59

### Synthesis of N-[4-(1-cyclopropanecarbonyl-2,3-dihydro-1H-indol-5-yl)-5-methyl-1,3-thiazol-2-yl]-2-{3-[(7-{[2-(2,6-dioxopiperidin-3-yl)-1,3-dioxo-2,3-dihydro-1H-isoindol-4-yl]amino}heptyl)oxy]phenyl}acetamide (Cpd. No. 71)

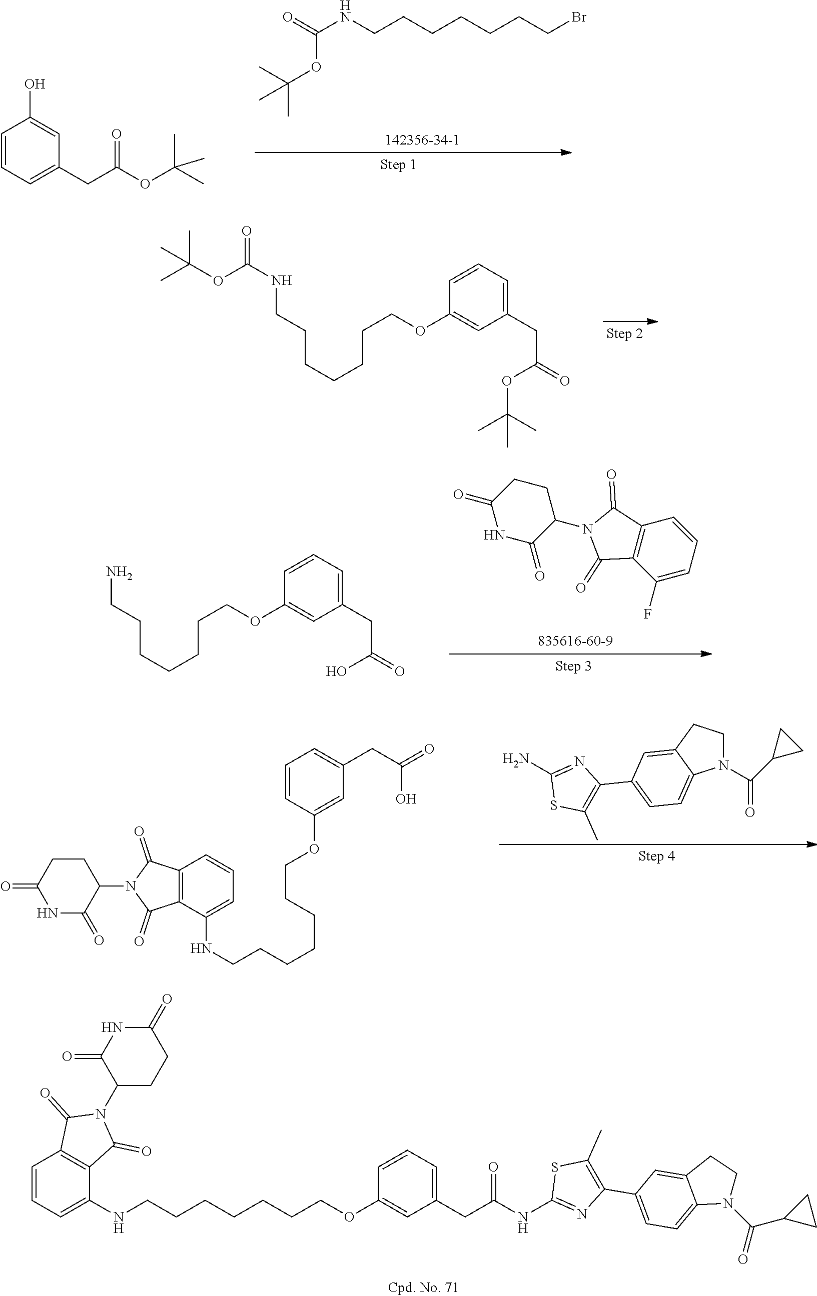

### Step 0: tert-butyl 2-{3-[(7-{[(tert-butoxy)carbonyl]amino}heptyl) oxy]phenyl}acetate

A mixture of tert-butyl 2-(3-hydroxyphenyl)acetate (0.5 g, 2.401 mmol, 1.0 eq), tert-butyl(7-bromoheptyl)carbamate (0.706 g, 2.401 mmol, 1.0 eq) and cesium carbonate (2.347 g, 7.203 mmol, 3.0 eq) in N,N-dimethylformamnide (17 ml, 0.14 M) was stirred at room temperature overnight. RM was evaporated and to the residue was added EA. Mixture was filtered and filter cake was washed thoroughly with EA. Filtrate was evaporated to give 1.033 g (quant. yield) of the crude title compound as a brown oil. LCMS: [C₂₄H₃₉NO₅], desired mass=421.578, observed mass=322.5 [M+H-Boc]⁺, ¹H NMR (300 MHz, Chloroform-d) δ 7.23 (t, J=7.8 Hz, 1H), 6.89-6.76 (m, 3H), 4.52 (s, 1H), 3.96 (t, J=6.5 Hz, 2H), 3.51 (s, 2H), 3.13 (q, J=6.7 Hz, 2H), 1.84-1.72 (m, 2H), 1.55-1.41 (m, 22H), 1.41-1.32 (m, 4H).

### Step 2: 2-{3-[(7-aminoheptyl)oxy]phenyl}acetic acid

To a solution of tert-butyl 2-{3-[(7-{[(tert-butoxy)carbonyl]amino}heptyl) oxy]phenyl}acetate (1.033 g, 2.401 mmol, 1.0 eq) in dichloromethane (16 ml, 0.14 M) was added trifluoroacetic acid (13.69 g, 120.066 mmol, 50 eq) and it was stirred at room temperature for 1 h. The solvent was removed in vacuo to give 1.04 g (quant. yield) of the title compound as a brown oil. Crude taken to the next step. LCMS: [C₅H₂₃NO₃], desired mass=265.353, observed mass=266.3 [M+H]⁺, ¹H NMR (300 MHz, Chloroform-d) δ 8.69 (s, 2H), 7.27-7.15 (m, 1H), 6.88-6.70 (m, 3.87 (s, 1H), 3.53 (s, 1H), 2.82 (s, 2H), 1.85-1.02 (m, 12H).

### Step 3: 2-{3-[(7-{[2-(2,6-dioxopiperidin-3-yl)-1,3-dioxo-2,3-dihydro-1H-isoindol-4-yl]amino}heptyl)oxy]phenyl}acetic acid

A mixture of 2-{3-[(7-aminoheptyl)oxy]phenyl}acetic acid (1.04 g, 2.391 mmol, 1.0 eq), 2-(2,6-Dioxo-piperidin-3-yl)-4-fluoroisoindoline-1,3-dione (1.387 g, 5.021 mmol, 2.1 eq) and N,N-Diisopropylethylamine (DIPEA) (2.166 ml, 12.432 mmol, 5.2 eq) in N-methyl-2-pyrrolidone (11.95 ml, 0.2 M) was stirred at 100° C. overnight. The mixture was cooled to room temperature, diluted with water, extracted with dichloromethane (×3), dried over anhydrous sodium sulfate, filtered and concentrated in vacuo. The residue was purified by RP FC (H₂O/ACN+0.1% FA 95:5 to 30:70, loaded as a residue of NMP with addition of water) and freeze-dried to give 0.572 g (46% yield) of the title compound as a yellow solid. LCMS: [C₂₈H₃₁N₃O₇], desired mass=521.57, observed mass=522.6 [M+H]⁺, ¹H NMR (300 MHz, DMSO-d₆) δ 11.09 (s, 1H), 7.58 (dd, J=8.6, 7.0 Hz, 1H), 7.19 (dd, J=8.9, 6.8 Hz, 1H), 7.10 (d, J=8.6 Hz, 1H), 7.02 (d, J=7.0 Hz, 1H), 6.85-6.75 (m, 3H), 6.54 (t, J=6.0 Hz, 1H), 5.05 (dd, J=12.8, 5.4 Hz, 1H), 3.93 (t, J=6.5 Hz, 2H), 3.51 (s, 2H), 3.25-3.16 (m, 2H), 2.95-2.81 (m, 1H), 2.67-2.53 (m, 2H), 2.11-1.96 (m, 1H), 1.76-1.64 (m, 2H), 1.65-1.53 (m, 2H), 1.39 (s, 6H).

### Step 4: N-[4-(1-cyclopropanecarbonyl-2,3-dihydro-1H-indol-5-yl)-5-methyl-1,3-thiazol-2-yl]-2-{3-[(7-{[2-(2,6-dioxopiperidin-3-yl)-1,3-dioxo-2,3-dihydro-1H-isoindol-4-yl]amino}heptyl)oxy]phenyl}acetamide

To a solution of 2-{3-[(7-{[2-(2,6-dioxopiperidin-3-yl)-1,3-dioxo-2,3-dihydro-1H-isoindol-4-yl]amino}heptyl)oxy]phenyl}acetic acid (0.138 g, 0.264 mmol, 1.4 eq) and 4-(1-cyclopropanecarbonyl-2,3-dihydro-1H-indol-5-yl)-5-methyl-1,3-thiazol-2-amine (0.06 g, 0.188 mmol, 1.0 eq) in a 1:1 mixture of Dimethylformamide anhydrous (3.8 ml, 0.05 M):Acetonitrile anhydrous (3.8 ml, 0.05 M) was added 1-Methylimidazole (NMI) (0.077 g, 0.942 mmol, 5 eq). To this reaction mixture at 0° C. was added N,N,N′,N′-Tetramethylchloroformaindinium hexafluorophosphate (TCFH)(0.159 g, 0.565 mmol, 3.0 eq) and the reaction mixture was stirred at r.t. over the weekend. Solvents were evaporated, and crude was extracted using DCM and bicarbonate solution. After drying over Na₂SO₄, crude was submitted for prep. HPLC (FA) to give 0.054 g (36% yield) of the title compound as a yellow solid. LCMS: [C₄₄H₄₆N₆O₇S], desired mass=802.95, observed mass=803.8 [M+H]⁺, ¹H NMR (300 MHz, DMSO-d₆) δ 12.28 (s, 1H), 11.09 (s, 1H), 8.15-7.93 (m, 1H), 7.62-7.53 (m, 1H), 7.50 (s, 1H), 7.42 (d, J=7.9 Hz, 1H), 7.22 (t, J=7.8 Hz, 1H), 7.08 (d, J=8.6 Hz, 1H), 7.01 (d, J=7.0 Hz, 1H), 6.93-6.85 (m, 2H), 6.86-6.77 (m, 1H), 6.52 (t, J=6.0 Hz, 1H), 5.04 (dd, J=12.8, 5.4 Hz, 1H), 4.38-4.24 (m, 2H), 3.95 (t, J=6.4 Hz, 2H), 3.69 (s, 2H), 3.28-3.13 (m, 5H), 2.96-2.79 (m, 1H), 2.64-2.49 (m, 2H), 2.43 (s, 3H), 2.07-1.92 (m, 2H), 1.77-1.63 (m, 2H), 1.65-1.51 (m, 2H), 1.38 (s, 5H), 0.94-0.80 (m, 4H).

### Example 60

### Synthesis of N-[4-(1-cyclopropanecarbonyl-2,3-dihydro-1H-indol-5-yl)-5-methyl-1,3-thiazol-2-yl]-2-{3-[(8-{[2-(2,6-dioxopiperidin-3-yl)-1,3-dioxo-2,3-dihydro-1H-isoindol-4-yl]amino}octyl)oxy]phenyl}acetamide (Cpd. No. 72)

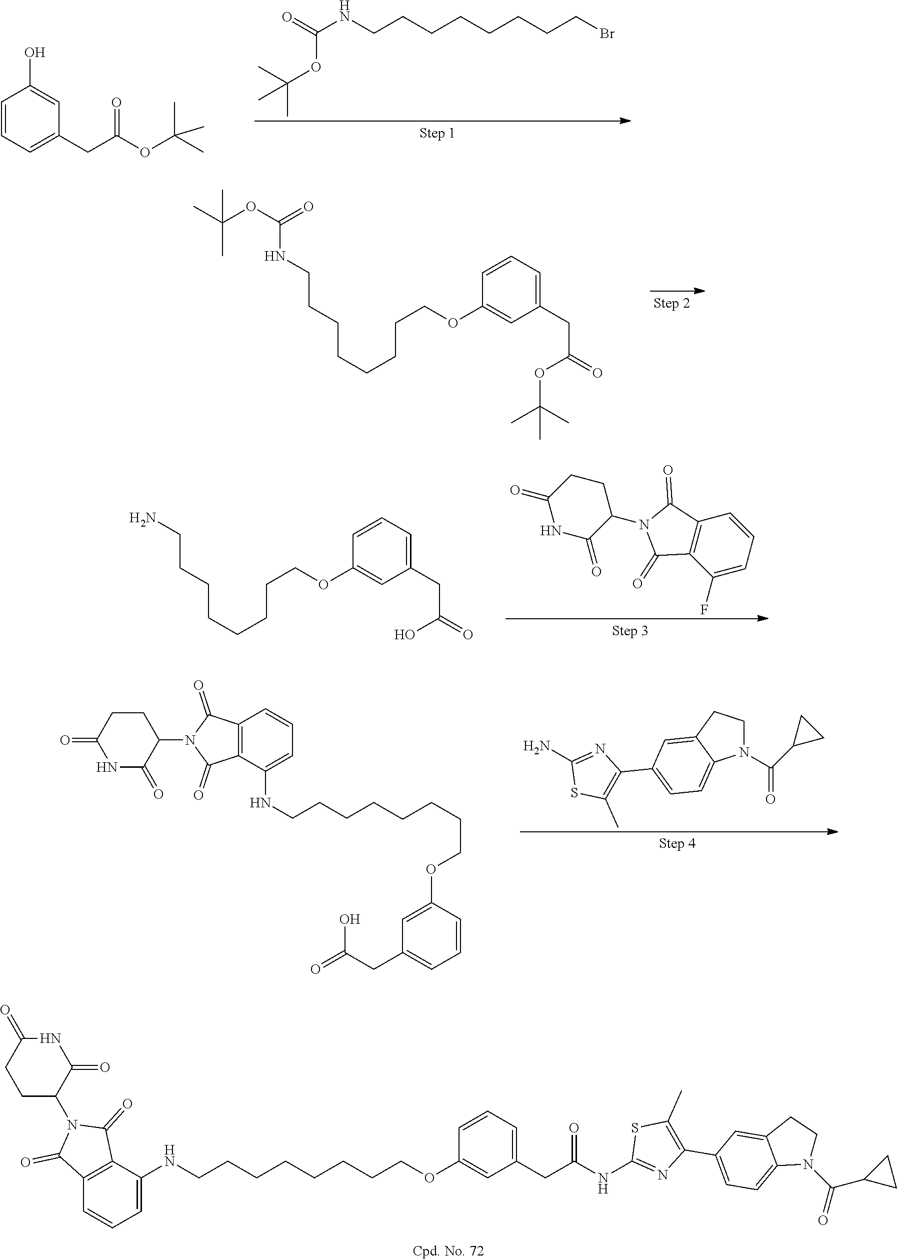

### Step 1: tert-butyl 2-{3-[(8-{[(tert-butoxy)carbonyl]amino}octyl)oxy]phenyl}acetate

A mixture of tert-butyl 2-(3-hydroxyphenyl)acetate (0.5 g, 2.401 mmol, 1.0 eq), N-Boc-8-bromooctan-1-amine(0.74 g, 2.4 mmol, 1.0 eq) and cesium carbonate (2.35 g, 7.2 mmol, 3.0 eq) in N,N-dimethylformamide (17 ml, 0.14 M) was stirred at room temperature overnight. RM was evaporated and to the residue Ethyl acetate was added. Mixture was filtered and filter cake was washed thoroughly with EtOAc. Filtrate was evaporated to give crude product 1.04 g as a brown oil that was used in next step without additional purification. ¹H NMR (300 MHz, Chloroform-d) δ 7.27-7.16 (m, 1H), 6.91-6.76 (m, 3H), 3.98-3.86 (m, 2H), 3.51 (s, 1H), 3.13 (t, J=7.1 Hz, 2H), 1.81-1.72 (m, 3H), 1.54-1.32 (m, 29H).

### Step 2: 2-{3-[(8-aminooctyl)oxy]phenyl}acetic acid

To a solution of tert-butyl 2-{3-[(8-{[(tert-butoxy)carbonyl]amino}octyl) oxy]phenyl}acetate (1.04 g, 2.39 mmol, 1.0 eq) in dichloromethane (16 ml, 0.14 M) was added trifluoroacetic acid (9.1 ml, 120.0 mmol, 50 eq) and stirred at room temperature for 1 h (Full deprotection confirmed by UPLC. The solvents were removed in vacuo to give title compound as a brown oil. Crude (1.23 g, 100% yield) was taken into the next step without additional purification. LCMS: [C₁₆H₂₅NO₃], desired mass=279.38, observed mass=280.4 [M+H]⁺,

### Step 3: 2-{3-[(8-{[2-(2,6-dioxopiperidin-3-yl)-1,3-dioxo-2,3-dihydro-H-isoindol-4-yl]amino}octyl)oxy]phenyl}acetic acid

A mixture of 2-{3-[(8-aminooctyl)oxy]phenyl}acetic acid (1.23 g, 2.38 mmol, 1.0 eq), 2-(2,6-Dioxo-piperidin-3-yl)-4-fluoroisoindoline-1,3-dione (1.4 g, 5.0 mmol, 2.1 eq) and N,N-Diisopropylethylamine (DIPEA) (2.15 ml, 12.36 mmol, 5.2 eq) in N-methyl-2-pyrrolidone (12 ml, 0.2 M) was stirred at 100 C for 18 hrs. The mixture was cooled to room temperature, diluted with water, extracted with dichloromethane (×4), dried over anhydrous sodium sulfate, filtered and concentrated in vacuo. The residue was purified by RP FC (H₂O/ACN 5-70%) loaded as a residue of NMP with addition of water to provide 1.18 g (88% yield) of title compound as a yellow solid. LCMS: [C₉H₃₃N₃O₇], desired mass=535.597, observed mass=536.6 [M+H]⁺, ¹H NMR (300 MHz, DMSO-d₆) δ 11.13 (d, J=16.9 Hz, 1H), 7.19 (t, J=7.7 Hz, 1H), 7.09 (d, J=8.6 Hz, 1H), 7.02 (d, J=7.0 Hz, 1H), 6.83-6.76 (m, 6.53 (t, J=5.9 Hz, 1H), 5.05 (dd, J=12.8, 5.4 Hz, 1H), 3.92 (q, J=3.6 Hz, 2H), 3.29 (t, J=6.7 Hz, 2H), 3.19-3.11 (m, 1H), 2.96-2.81 (m, 2H), 2.69-2.56 (m, 3H), 2.09-1.98 (m, 1H), 1.64 (dt, J=34.2, 6.9 Hz, 4H), 1.05 (d, J=6.5 Hz, 8H).

### Step 4: N-[4-(1-cyclopropanecarbonyl-2,3-dihydro-1H1-indol-5-yl)-5-methyl-1,3-thiazol-2-yl]-2-{3-[(8-{[2-(2,6-dioxopiperidin-3-yl)-1,3-dioxo-2,3-dihydro-1H-isoindol-4-yl]amino}octyl)oxy]phenyl}acetamide

A mixture of 4-(1-cyclopropanecarbonyl-2,3-dihydro-1H-indol-5-yl)-5-methyl-1,3-thiazol-2-amine (0.06 g, 0.188 mmol, 1.0 eq), 2-{3-[(8-{[2-(2,6-dioxopiperidin-3-yl)-1,3-dioxo-2,3-dihydro-1H-isoindol-4-yl]amino}octyl)oxy]pbenyl}acetic acid (0.150 g, 0.26 mmol, 1.4 eq), and N,N,N′,N′-Tetramethylchloroformamidinium hexafluorophosphate (0.16 g, 0.57 mmol, 3.0 eq) in anh THF/ACN (1:1) (0.05 M) was stirred for 10 mins. Then 1-Methylimidazole (0.077 g, 0.94 mmol, 5.0 eq) was added and reaction was stirred at room temperature overnight. UPLC showed formation of DP. Solvents were evaporated and crude was dissolved in DCM and aqueous NaHCO₃ was added. Mixture was stirred for 20 mins and phases were separated. Organic phase was dried under Na₂SO₄ and evaporated to dryness. Crude was submitted for prep HPLC. 43.68 mg (27% yield) was acquired of title compound as a bright yellow solid. LCMS: [C₄₅H₄₈N₆O₇S], desired mass=816.97, observed mass=817.43 [M+H]⁺, ¹H NMR (300 MHz, DMSO-d6) δ 12.28 (s, 1H), 11.09 (s, 1H), 8.06 (s, 1H), 7.63-7.53 (m, 1H), 7.49 (s, 1H), 7.42 (d, J=8.2 Hz, 1H), 7.22 (t, J=7.8 Hz, 1H), 7.08 (d, J=8.6 Hz, 1H), 7.01 (d, J=7.0 Hz, 1H), 6.93-6.77 (m, ³H), 6.52 (t, J=5.9 Hz, 1H), 5.04 (dd, J=12.8, 5.4 Hz, 1H), 4.32 (s, 2H), 3.94 (t, J=6.4 Hz, 2H), 3.69 (s, 2H), 3.33-3.07 (m, 2.96-2.78 (m, 1H), 2.43 (s, 3H), 2.07-1.86 (m, 2H), 1.75-1.51 (m, 1.45-1.25 (m, 8H), 0.93-0.81 (m, 4H).

### Example 61

### Synthesis of 4-(1-cyclopropanecarbonyl-2,3-dihydro-1H-indol-5-yl)-N-({3-[(5-{[2-(2,6-dioxopiperidin-3-yl)-1,3-dioxo-2,3-dihydro-1H-isoindol-4-yl]amino}pentyl)oxy]phenyl}methyl)-5-methyl-1,3-thiazole-2-carboxamide (Cpd. No. 73)

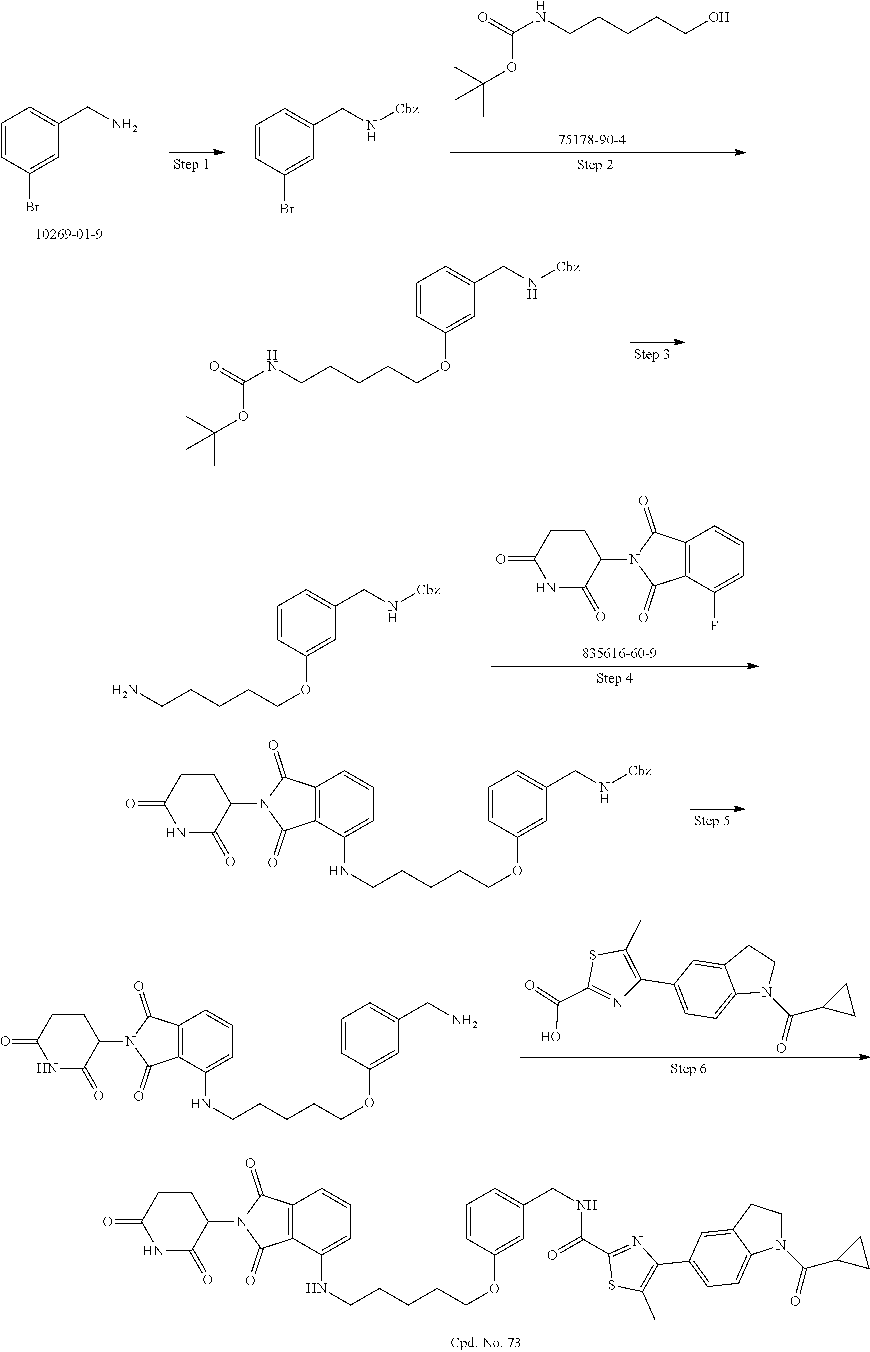

### Step 1: benzyl N-[(3-bromophenyl)methyl]carbamate

To a solution of 3-Bromobenzylamine (1.0 g, 5.37 mmol, 1.0 Equiv.) in anh. DCM (13.4 ml, 0.4 M) was added TEA (0.9 ml, 6.45 mmol, 1.2 Equiv.) and reaction mixture was cooled down to 0° C. Next benzyl chloroformate (0.9 ml, 6.45 mmol, 1.2 Equiv.) was added dropwise and RM was stirred at 0° C. for additional 10 min and then at rt overnight. RM was diluted with DCM, washed with water (2×) and brine, dried over Na₂SO₄, filtered and concentrated under vacuo. Crude was used in next step without additional purification. LCMS: [C₁₅H₁₄BrNO₂], desired mass=320.186, observed mass=319.3 [M+H]⁺, ¹H NMR (300 MHz, DMSO-d6) δ 7.40-7.23 (m, 10H), 5.06 (s, 2H), 4.22 (t, J=6.4 Hz, 2H).

### Step 2: benzyl N-({3-[(5-{[(tert-butoxy)carbonyl]amino}pentyl)oxy]phenyl}methyl)carbamate

To an argon flushed pressure vessel containing benzyl N-[(3-bromophenyl)methyl]carbamate (0.1 g, 0.28 mmol, 1.0 eq), Cs₂CO₃ (0.138 g, 0.42 mmol, 1.5 eq) and 5-(Boc-amino)-1-pentanol (0.114 g, 0.56 mmol, 2.0 eq) in toluene (0.6 ml, 0.5 M) was added RockPhos Pd G3 (0.005 g, 0.006 mmol, 0.02 eq). Then the reaction was proceeded at 90° C. overnight. UPCL showed formation of DP. Reaction was quenched with water and extracted with EtOAc. Organic phase was dried over Na₂SO₄ and evaporated to dryness. Crude was purified by DCM:EtOAc (0-90%) to provide 0.79 g (76% yield) of title compound as a brown solid. LCMS: [C₂₅H₃₄N₂O₅], desired mass 442.556, observed mass=443.5 [M+H]⁺, ¹H NMR (300 MHz, DMSO-d6) δ 7.40-7.26 (m, 51), 7.21 (t, J=7.9 Hz, 1H), 6.88-6.73 (m, 3H), 5.05 (s, 21), 4.15 (dd, J=11.8, 6.0 Hz, 3H), 3.91 (t, J=6.4 Hz, 1H), 2.93 (q, J=6.3 Hz, 2H), 1.69 (t, J=6.8 Hz, 2H), 1.41 (s, 9H), 0.86 (q, J=6.8 Hz, 3H).

### Step 3: benzyl N-({3-[(5-aminopentyl)oxy]phenyl}methyl)carbamate; tris(trifluoroacetic acid)

Benzyl N-({3-[(5-{[(tert-butoxy)carbonyl]amino}pentyl)oxy]phenyl}methyl) carbamate (0.79 g, 1.43 mmol, 1.0 eq) was dissolved in anh. DCM (7.1 mL, 0.2 M) followed by addition of TFA (0.63 ml, 50.0 mmol, 35.0 eq) and stirred at RT for 2 hours. UPLC showed full conversion. The solvents were removed and the residue triturated with Et₂O to give 920 mg (100% yield) of title compound as a orange oil. LCMS: [C₂₆H₂₉F₉N₂O₉], desired mass=684.509, observed mass=343.5 [M+H]⁺,

### Step 4: benzyl N-({3-[(5-{[2-(2,6-dioxopiperidin-3-yl)-1,3-dioxo-2,3-dihydro-1H-isoindol-4-yl]amino}pentyl)oxy]phenyl}methyl)carbamate

A mixture of benzyl N-({3-[(5-aminopentyl)oxy]phenyl}methyl)carbamate; tris(trifluoroacetic acid) (0.92 g, 1.34 mmol, 1.0 eq), 2-(2,6-Dioxo-piperidin-3-yl)-4-fluoroisoindoline-1,3-dione (0.78 g, 2.8 mmol, 2.1 eq) and N,N-Diisopropylethylamine (DIPEA) (1.21 ml, 7.0 mmol, 5.2 eq) in N-methyl-2-pyrrolidone (12 ml, 0.2 M) was stirred at 100 C for 18 hrs. The mixture was cooled to room temperature, diluted with water, extracted with dichloromethane (×4), dried over anhydrous sodium sulfate, filtered and concentrated in vacuo. The residue was purified by RP FC (H₂O/ACN 5-70%) loaded as a residue of NMP with addition of water to provide 0.46 g (58% yield) of title compound as a yellow solid. LCMS: [C₃₃H₃₄N₄O₇], desired mass=598.656, observed mass=599.6 [M+H]⁺, ¹H NMR (300 MHz, DMSO-d6) δ 11.09 (s, 1H), 7.79 (t, J 6.2 Hz, 1H), 7.64-7.52 (m, 1H), 7.36 (d, J=3.8 Hz, 5H), 7.20 (t, J=7.9 Hz, 1H), 7.11 (d, J=8.6 Hz, 1H), 7.02 (d, J=7.0 Hz, 1H), 6.79 (t, J=6.0 Hz, 3H), 6.57 (t, J=5.9 Hz, 1H), 5.09-4.99 (m, 3H), 4.16 (d, J=6.2 Hz, 2H), 3.93 (t, J=6.3 Hz, 2H), 2.93-2.82 (m, 1H), 2.63-2.53 (m, 2H), 2.05-1.97 (m, 1H), 1.79-1.42 (m, 7H).

### Step 5: 4-({5-[3-(aminomethyl)phenoxy]pentyl}amino)-2-(2,6-dioxopiperidin-3-yl)-2,3-dihydro-1H-isoindole-1,3-dione; bis(trifluoroacetic acid)

Benzyl N-({3-[(5-{[2-(2,6-dioxopiperidin-3-yl)-1,3-dioxo-2,3-dihydro-1H-isoindol-4-yl]amino}pentyl)oxy]phenyl}methyl)carbamate (0.443 g, 0.74 mmol, 1.0 eq) was dissolved in 5.1M HBr solution in AcOH (2.2 ml, 11.1 mmol, 15.0 eq) and reaction was stirred for 1 h at rt. UPLC showed full deprotection. Solvents were evaporated to provide 0.48 g of title compound as orange oil. LCMS: [C₂₉H₃₀F₆N₄O₉], desired mass=692.568, observed mass=465.5 [M+H]⁺,

### Step 6: 4-(1-cyclopropanecarbonyl-2,3-dihydro-1H-indol-5-yl)-N-({3-[(5-{[2-(2,6-dioxopiperidin-3-yl)-1,3-dioxo-2,3-dihydro-1H-isoindol-4-yl]amino}pentyl)oxy]phenyl}methyl)-5-methyl-1,3-thiazole-2-carboxamide

A mixture of 4-({5-[3-(aminomethyl)phenoxy]pentyl}amino)-2-(2,6-dioxopiperidin-3-yl)-2,3-dihydro-1H-isoindole-1,3-dione; bis(trifluoroacetic acid) (0.12 g, 0.17 mmol, 1.0 eq), 4-(1-cyclopropanecarbonyl-2,3-dihydro-1-H-indol-5-yl)-5-methyl-1,3-thiazole-2-carboxylic acid (0.065 g, 0.2 mmol, 1.1 eq), and 1-Methylimidazole (0.07 ml, 0.86 mmol, 5.0 eq) in anh THF/ACN (1:1) (0.4 M) was stirred for 10 mins. Then N,N,N′,N′-Tetramethylchloroformamidinium hexafluorophosphate (0.15 g, 0.52 mmol, 3.0 eq) was added and reaction was stirred at room temperature overnight. UPLC showed formation of DP. Solvents were evaporated and crude was dissolved in DCM and aqueous NaHCO₃ was added. Mixture was stirred for 20 mins and phases were separated. Organic phase was dried over Na₂SO₄ and evaporated to dryness. Crude was submitted for prep HPLC. 19.74 mg (15% yield) was acquired of title compound as a bright yellow solid. LCMS: [C₄₂H₄₂N₆O₇S], desired mass=774.89, observed mass=775.36 [M+H]⁺, ¹H NMR (300 MHz, DMSO-d6) δ 11.08 (s, 1H), 9.26 (t, J=6.3 Hz, 1H), 8.09 (s, 1H), 7.65 (s, 1H), 7.55 (d, J=7.1 Hz, 2H), 7.22 (t, J=8.1 Hz, 1H), 7.08 (s, 1H), 7.01 (d, J=7.0 Hz, 1H), 6.88 (d, J=7.0 Hz, 2H), 6.81 (d, J=7.7 Hz, 1H), 6.55 (t, J=6.0 Hz, 1H), 5.04 (dd, J=12.8, 5.4 Hz, 1H), 4.45-4.28 (m, 3.95 (t, J=6.4 Hz, 2H), 3.24-3.18 (m, 2H), 2.86 (dd, J=17.3, 5.1 Hz, 2H), 2.60 (s, 3H), 2.57-2.52 (m, 2H), 2.02 (d, J=13.8 Hz, 2H), 1.81-1.41 (m, 7H), 0.88 (d, J=8.0 Hz, 4H).

### Example 62

### Synthesis of N-[4-(1-cyclopropanecarbonyl-2,3-dihydro-1H-indol-5-yl)-5-methyl-1,3-thiazol-2-yl]-2-{3-[(5-{[2-(2,6-dioxopiperidin-3-yl)-1,3-dioxo-2,3-dihydro-1H-isoindol-4-yl]amino}pentyl)oxy]phenyl}acetamide (Cpd. No. 40)

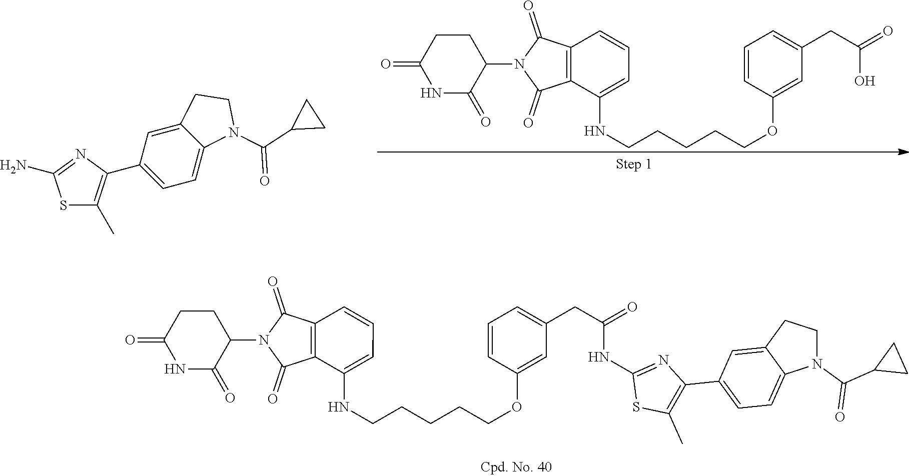 [SMILES: Cc1sc(N)nc1-c1ccc2c(c1)CCN2C(=O)C1CC1 ; O=C(O)Cc1cccc(OCCCCCNc2cccc3c2C(=O)N(C2CCC(=O)NC2=O)C3=O)c1 ; Cc1sc(NC(=O)Cc2cccc(OCCCCCNc3cccc4c3C(=O)N(C3CCC(=O)NC3=O)C4=O)c2)nc1-c1ccc2c(c1)CCN2C(=O)C1CC1]

### Step 1: N-[4-(1-cyclopropanecarbonyl-2,3-dihydro-1H-indol-5-yl)-5-methyl-1,3-thiazol-2-yl]-2-{3-[(5-{[2-(2,6-dioxopiperidin-3-yl)-1,3-dioxo-2,3-dihydro-1H-isoindol-4-yl]amino}pentyl)oxy]phenyl}acetamide

A mixture of 4-(1-cyclopropanecarbonyl-2,3-dihydro-1H-indol-5-yl)-5-methyl-1,3-thiazol-2-amine (0.08 g, 0.262 mmol, 1.0 eq), 1-Methylimidazole (0.108 g, 1.309 mmol, 5.0 eq) and 2-{3-[(5-{[2-(2,6-dioxopiperidin-3-yl)-1,3-dioxo-2,3-dihydro-1H-isoindol-4-yl]amino}pentyl)oxy]phenyl}acetic acid (0.147 g, 0.288 mmol, 1.1 eq) in anh DMF (0.65 ml, 0.4 M) and anh ACN (0.65 ml, 0.4 M) was cooled to 0° C. and then TCFH (0.22 g, 0.786 mmol, 3.0 eq) was added. The RM was stirred at rt for overnight. UPLC analysis showed 90% conversion. The solvents were evaporated to dryness and obtained material was dissolved in DCM, aq sat. NaHCO₃ was added and it was stirred for 30 minutes at rt. It was extracted 3× with DCM, dried over Na₂SO₄ and evaporated to dryness. Then it was purified by prep. HPLC (acidic conditions with FA) to give 79 mg (39% yield) of the title compound as a yellow solid. LCMS: [C₄₂H₄₂N₆O₇S], desired mass=774.9, observed mass=775.3 [M+H]⁺, ¹H NMR (400 MHz, DMSO) δ 12.29 (s, 1H), 11.10 (s, 1H), 8.07 (d, J=8.4 Hz, 1H), 7.57 (dd, J=8.6, 7.1 Hz, 1H), 7.50 (s, 1H), 7.43 (d, J=8.8 Hz, 1H), 7.23 (t, J=7.9 Hz, 1H), 7.11 (d, J=8.6 Hz, 1H), 7.02 (d, J=7.0 Hz, 1H), 6.94-6.86 (m, 2H), 6.85-6.81 (m, 1H), 6.58 (t, J=6.0 Hz, 1H), 5.06 (dd, J=12.9, 5.4 Hz, 1H), 4.40-4.27 (m, 2H), 3.97 (t, J=6.4 Hz, 2H), 3.70 (s, 2H), 3.23 (t, J=8.7 Hz, 2H), 2.88 (ddd, J=18.0, 14.0, 5.4 Hz, 1H), 2.64-2.52 (m, 2.44 (s, 3H), 2.08-1.93 (m, 2H), 1.77 (p, J=6.6 Hz, 2H), 1.65 (p, J=7.2 Hz, 2H), 1.55-1.46 (m, 2H), 0.94-0.83 (m, 4H).

### Example 63

### Synthesis of N-[4-(1-acetyl-2,3-dihydro-1H-indol-5-yl)-5-methyl-1,3-thiazol-2-yl]-2-{3-[(5-{[2-(2,6-dioxopiperidin-3-yl)-1,3-dioxo-2,3-dihydro-1H-isoindol-4-yl]amino}pentyl)oxy]phenyl}acetamide (Cpd. No. 67)

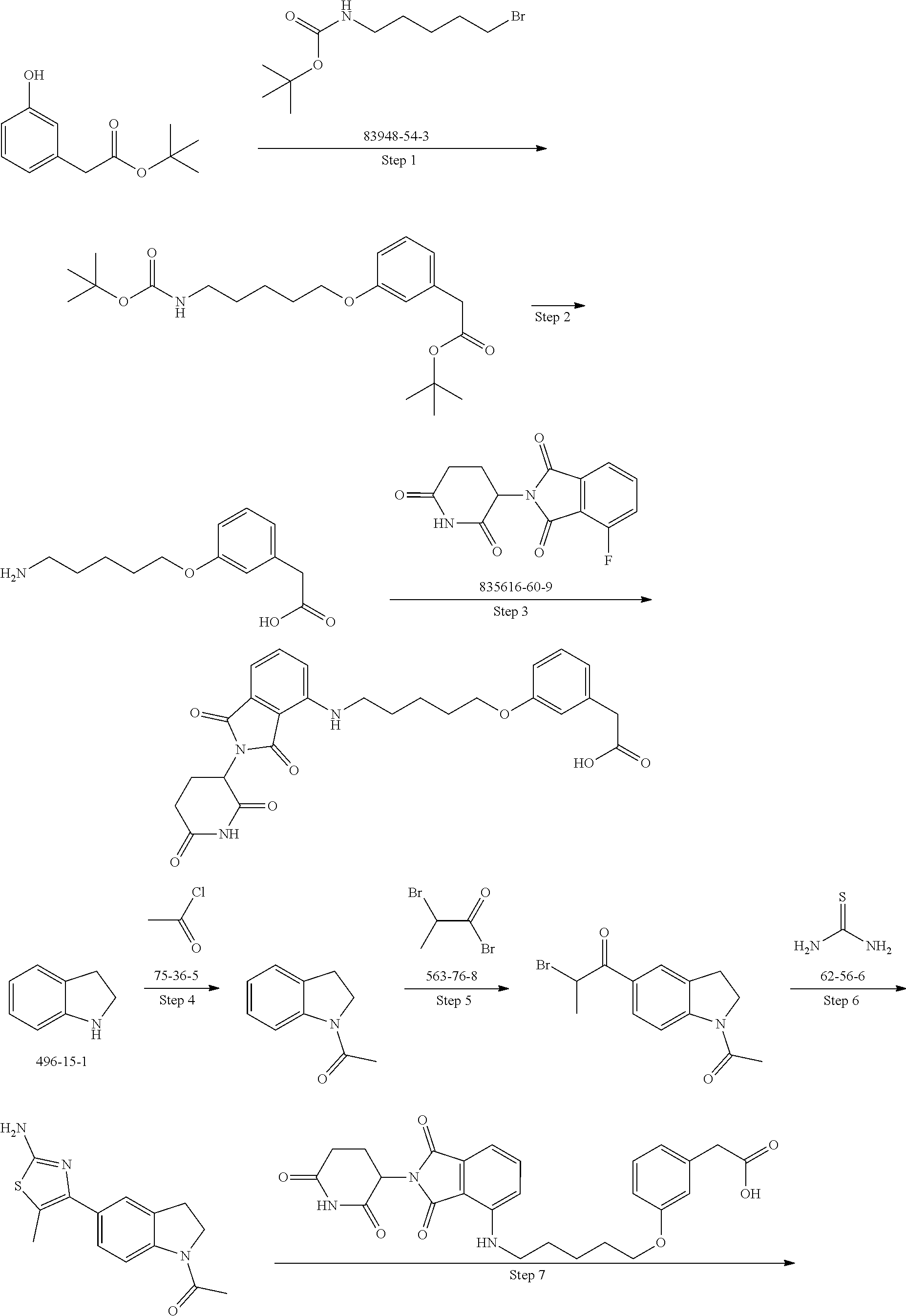

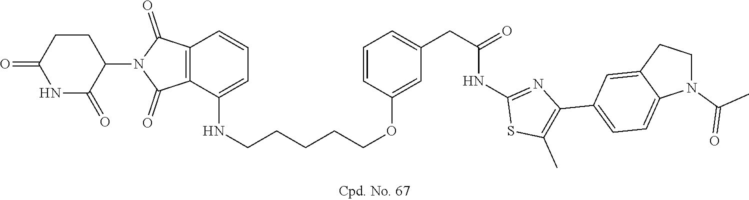 [SMILES: CC(=O)N1CCc2cc(-c3nc(NC(=O)Cc4cccc(OCCCCCNc5cccc6c5C(=O)N(C5CCC(=O)NC5=O)C6=O)c4)sc3C)ccc21]

### Step 1: tert-butyl 2-{3-[(5-{[(tert-butoxy)carbonyl]amino}pentyl)oxy]phenyl}acetate

A mixture of tert-butyl 2-(3-hydroxyphenyl)acetate (1.0 g, 4.562 mmol, 1.0 eq), 5-(Boc-amino)-1-pentyl bromide (1.214 g, 4.562 mmol, 1.0 eq) and cesium carbonate (4.459 g, 13.685 mmol, 3.0 eq) in N,N-dimethylformamide (32.6 ml, 0.14 M) was stirred at room temperature overnight. RM was evaporated and to the residue was added EtOAc. Mixture was filtered and filter cake was washed thoroughly with EA. Filtrate was evaporated to give crude product tert-butyl 2-{3-[(5-{[(tert-butoxy)carbonyl]amino}pentyl)oxy]phenyl}acetate (1.761 g, 4.475 mmol, 98%) as a brown oil. LCMS: [C₂₂H₃₅NO₅], desired mass=393.524, observed mass=394.25 [M+H]⁺, ¹H NMR (300 MHz, Chloroform-d) δ 7.23 (t, J=7.8 Hz, 1H), 6.89-6.77 (m, 3H), 4.56 (s, 1H), 3.97 (t, J=6.4 Hz, 2H), 3.51 (s, 2H), 3.17 (q, J=6.3 Hz, 2H), 1.82 (p, J=6.6 Hz, 2H), 1.59-1.50 (m, 1.47 (d, J=1.9 Hz, 18H).

### Step 2: 2-{3-[(5-aminopentyl)oxy]phenyl}acetic acid; trifluoroacetic acid

tert-butyl 2-{3-[(5-{[(tert-butoxy)carbonyl]amino}pentyl)oxy]phenyl}acetate (1.761 g, 4.475 mmol, 1.0 eq) was dissolved in a mixture of DCM (11.19 ml, 0.4 M) and TFA (15.307 g, 134.248 mmol, 30.0 eq), and it was stirred at rt for 1 h. UPLC analysis showed full conversion. It was evaporated on rotavap, dissolved a few times in DCM and then in ACN, followed by evaporation after every addition of the solvent and evaporated to dryness. It was taken to the next step without purification. 1.874 g of the crude material as a viscous oil with quantitative yield was obtained. LCMS: [C₁₅H₂₀F₃NO₅], desired mass=351.322, observed mass=238.3 [M+H]⁺, ¹H NMR (300 MHz, DMSO) δ 7.67 (s, 3H), 7.22 (dd, J=9.4, 7.1 Hz, 1H), 6.88-6.76 (m, 3H), 4.23-4.18 (m, 1H), 3.95 (t, J=6.3 Hz, 2H), 3.53 (s, 2H), 2.88-2.74 (m, 2H), 1.82-1.67 (m, 2H), 1.65-1.55 (m, 2H), 1.54-1.43 (m, 2H).

### Step 3: 2-{3-[(5-{[2-(2,6-dioxopiperidin-3-yl)-1,3-dioxo-2,3-dihydro-1H-isoindol-4-yl]amino}pentyl)oxy]phenyl}acetic acid

A mixture of 2-(2,6-Dioxo-piperidin-3-yl)-4-fluoroisoindoline-1,3-dione (1.909 g, 6.911 mmol, 1.561 eq), 2-{3-[(5-aminopentyl)oxy]phenyl}acetic acid; trifluoroacetic acid (1.874 g, 4.428 mmol, 1.0 eq) and DIPEA (4.011 ml, 23.027 mmol, 5.2 eq) in NMP (14.76 ml, 0.3 M) was stirred at 100° C. for overnight. UPLC analysis showed full conversion. DIPEA was evaporated, and the solution in NMP was injected on the column for RPFC. It was purified in acidic conditions with 0.1% FA and eluted with ACN:H₂O (10-59% of ACN) to give 1.195 g (53% yield) of the title compound as a green solid. LCMS: [C₂₆H₂₇N₃O₇], desired mass=493.516, observed mass=494.3 [M+H]⁺, ¹H NMR (300 MHz, DMSO) δ 12.49 (s, 1H), 11.09 (s, 1H), 7.58 (dd, J=8.6, 7.1 Hz, 1H), 7.20 (dd, J=8.7, 7.1 Hz, 1H), 7.12 (d, J=8.6 Hz, 1H), 7.02 (d, J=7.0 Hz, 1H), 6.85-6.74 (m, 3H), 6.58 (t, J=6.1 Hz, 1H), 5.05 (dd, J=12.8, 5.4 Hz, 1H), 3.95 (t, J=6.3 Hz, 2H), 3.51 (s, 2H), 2.95-2.79 (m, 1H), 2.63-2.52 (m, 3H), 2.07-1.96 (m, 1H), 1.81-1.58 (m, 5H), 1.55-1.45 (m, 2H).

### Step 4: 1-(2,3-dihydro-1H-indol-1-yl)ethan-1-one

To a solution of indoline (5.00 g, 42.0 mmol) and triethylamine (12.74 g, 125.9 mmol) in dichloromethane anhydrous (150 mL, 30 vol) was added Acetyl chloride (3.133 ml, 44.056 mmol, 1.05 eq) at 0 C. After addition, the mixture was stirred at room temperature for 18 hrs. The mixture was quenched with water (50 mL), extracted with dichloromethane (50 mL), washed with saturated ammonium chloride aqueous solution (50 mL×2), dried over anhydrous sodium sulfate, filtered and concentrated in vacuo to give crude 1-(2,3-dihydro-1H-indol-1-yl)ethan-1-one (6.76 g, 41.95 mmol) as a brown solid. LCMS: [C₁₀H₁₁NO], desired mass=161.204, observed mass=162.1 [M+H]⁺, ¹H NMR (300 MHz, DMSO-d6) δ 8.04 (d, J=8.0 Hz, 1H), 7.29-7.19 (m, 1H), 7.19-7.08 (m, 1H), 6.98 (td, J=7.4, 1.2 Hz, 1H), 4.07 (q, J=7.9, 7.3 Hz, 2H), 3.14 (t, J=8.5 Hz, 3H), 2.16 (s, 3H).

### Step 5: 2-bromo-1-[1-(1-hydroxyethyl)-2,3-dihydro-1H-indol-5-yl]ethan-1-ol

1-(2,3-dihydro-1H-indol-1-yl)ethan-1-one (6.76 g, 41.934 mmol, 1.0 eq) and Aluminum chloride anhydrous (16.773 g, 125.803 mmol, 3.0 eq) was dissolved in dichloromethane (150 mL, 22 vol), then 2-Bromopropionyl bromide (27.157 g, 125.803 mmol, 3.0 eq) was added. The mixture was stirred at 50° C. for 4 hrs. The mixture was quenched with water (200 mL), basified to pH=8-10 with 6N sodium hydroxide solution, extracted with dichloromethane (600 mL), dried over anhydrous sodium sulfate, filtered and concentrated in vacuo to give crude 1-(1-acetyl-2,3-dihydro-1H-indol-5-yl)-2-bromoethan-1-one (12.0 g, 41.93 mmol, 97%) as a yellow oil. LCMS: [C₁₂H₁₆BrNO₂], desired mass=286.169, observed mass=298.24 [M+H]⁺, ¹H NMR (300 MHz, DMSO-d6) δ 8.11 (d, J=8.5 Hz, 1H), 7.96-7.80 (m, 2H), 5.76 (t, J=6.5 Hz, 1H), 4.16 (t, J=8.5 Hz, 2H), 3.19 (t, J=8.6 Hz, 2H), 2.20 (s, 3H), 1.76 (d, J=6.5 Hz, 3H).

### Step 6: 1-[5-(2-amino-5-methyl-1,3-thiazol-4-yl)-2,3-dihydro-1H-indol-1-yl]ethan-1-one

2-bromo-1-[1-(1-hydroxyethyl)-2,3-dihydro-1H-indol-5-yl]ethan-1-ol (12 g, 41.68 mmol, 1.0 eq) was dissolved in ethanol (180 ml, 15.0 vol) and thiourea (7.932 g, 104.2 mmol, 2.5 eq) was added as one portion at room temperature. Then reaction mixture was heated up to 78° C. and stirring was continued overnight. Next day, UPLC showed full conversion toward DP. Ethanol was evaporated and residue was suspended in DCM. After stirring for 20 min at RT, precipitate was filtered off. Then purification via automated flash chromatography (DCM:MeOH 95:5) was performed. 4-(1-cyclopropanecarbonyl-2,3-dihydro-1H-indol-5-yl)-5-methyl-1,3-thiazol-2-amine (2.95 g, 9.656 mmol, 95%) was isolated as a beige solid. LCMS: [C₁₄H₁₅N₃OS], desired mass=273.35, observed mass=274.31 [M+H]⁺, ¹H NMR (300 MHz, DMSO-d6) δ 8.85 (s, 2H), 8.13 (d, J=8.4 Hz, 1H), 7.39 (d, J=1.8 Hz, 1H), 7.31 (dd, J=8.4, 2.0 Hz, 1H), 4.15 (t, J=8.5 Hz, 2H), 3.26-3.14 (m, 2H), 2.27 (s, 3H), 2.19 (s, 3H).

### Step 7: N-[4-(1-acetyl-2,3-dihydro-1H-indol-5-yl)-5-methyl-1,3-thiazol-2-yl]-2-{3-[(5-{[2-(2,6-dioxopiperidin-3-yl)-1,3-dioxo-2,3-dihydro-1H-isoindol-4-yl]amino}pentyl)oxy]phenyl}acetamide

A mixture of 232800124-MIA01-004-F2 (0.08 g, 0.291 mmol, 1.0 eq), 1-Methylimidazole (0.119 g, 1.453 mmol, 5.0 eq) and 232800124-KLI01-022-RPFC (0.163 g, 0.32 mmol, 1.1 eq) in anh Dimethylformamide anhydrous (0.83 ml, 0.35 M) and anh Acetonitrile anhydrous (1.16 ml, 0.25 M) was cooled to 0° C. and then N,N,N′,N′-Tetramethylchloroformamidinium hexafluorophosphate (0.245 g, 0.872 mmol, 3.0 eq) was added and the RM was stirred at rt for overnight. The solvents were evaporated to dryness and obtained material was dissolved in DCM, aq NaHCO₃ was added and it was stirred for 30 minutes at rt. It was extracted 3× with DCM, dried over Na₂SO₄ and evaporated to dryness. Then it was purified by prepHPLC to give 78.6 mg (38% yield) of the title compound as an yellow solid. LCMS: [C₄₀H₄₀N₆O₇S], desired mass=748.86, observed mass=749.76 [M+H]⁺, ¹H NMR (300 MHz, DMSO) δ 12.29 (s, 1H), 11.10 (s, 1H), 8.09 (d, J=8.4 Hz, 1H), 7.58 (dd, J=8.6, 7.1 Hz, 1H), 7.49 (s, 1H), 7.47-7.39 (m, 1H), 7.23 (t, J=7.9 Hz, 1H), 7.11 (d, J=8.6 Hz, 1H), 7.02 (d, J=7.0 Hz, 1H), 6.96-6.84 (m, 2H), 6.83 (dd, J=8.2, 2.5 Hz, 1H), 6.58 (s, 1H), 5.06 (dd, J=12.8, 5.4 Hz, 1H), 4.13 (t, J=8.5 Hz, 2H), 3.97 (t, J=6.3 Hz, 2H), 3.70 (s, 2H), 3.33 (s, 2H), 3.19 (t, J=8.5 Hz, 2H), 2.89 (ddd, J=17.9, 13.7, 5.3 Hz, 1H), 2.62 (s, 1H), 2.55 (s, 1H), 2.44 (s, 3H), 2.18 (s, 3H), 2.03 (d, J=11.6 Hz, 1H), 1.77 (t, J=7.3 Hz, 2H), 1.76-1.58 (m, 2H), 1.53 (s, 2H). Remarks NMR: 0.17% wt of DCM, 0.15% wt of CH₃CN, 0.12% wt of FA

### Example 64

### Synthesis of N-[4-(1-acetyl-7-fluoro-2,3-dihydro-1H-indol-5-yl)-5-methyl-1,3-thiazol-2-yl]-2-{3-[(5-{[2-(2,6-dioxopiperidin-3-yl)-1,3-dioxo-2,3-dihydro-1H-isoindol-4-yl]amino}pentyl)oxy]phenyl}acetamide (Cpd. No. 75)

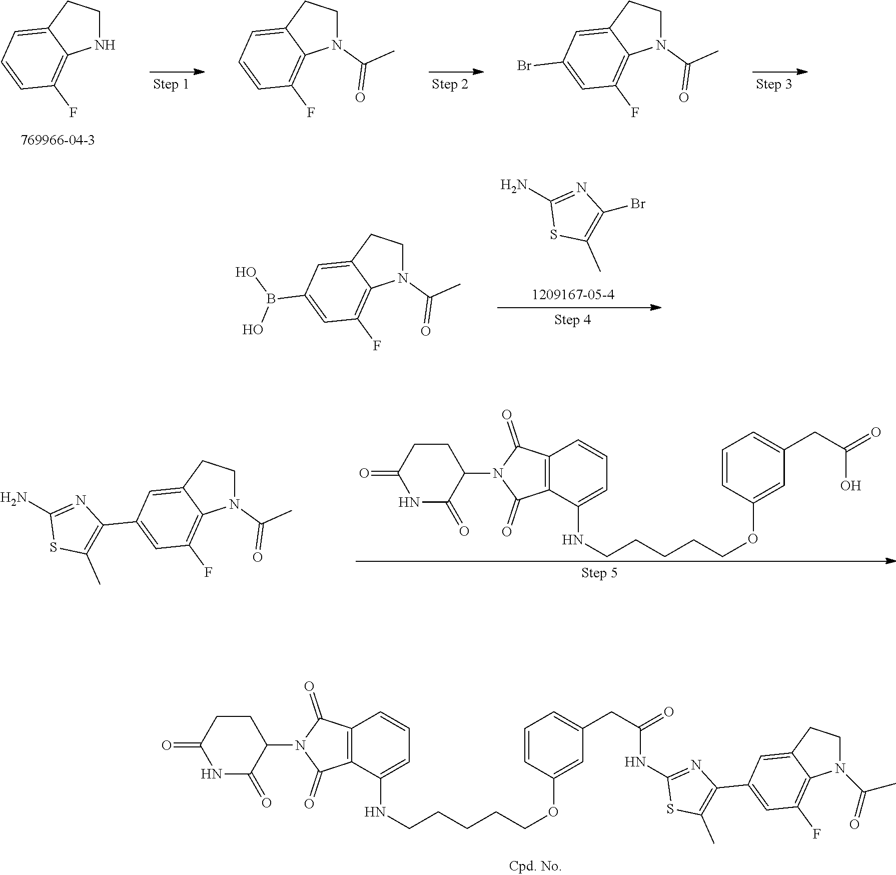

### Step 1: 1-(7-fluoro-2,3-dihydro-1H-indol-1-yl)ethan-1-one

To a solution of 7-fluoroindoline (1.00 g, 7.29 mmol) and triethylamine (3.05 ml, 21.87 mmol) in chloroform anhyd. (30 mL, 30 vol) was added Acetyl chloride (0.54 ml, 7.66 mmol, 1.05 eq) at 0° C. After addition, the mixture was stirred at rt on. The mixture was quenched with water (10 mL), extracted with DCM (10 mL), washed with sat. ammonium chloride aqueous solution (10 mL×2), dried over anhyd. sodium sulfate, filtered and concentrated in vacuo to give crude 1-(7-fluoro-2,3-dihydro-1H-indol-1-yl)ethan-1-one (1.016 g, 5.67 mmol, 78%) as a brown solid. LCMS: [C₁₀H₁₀FNO], desired mass=179.19, observed mass=180.2 [M+H]⁺, 1H NMR (300 MHz, CDCl₃) δ 7.10-6.91 (m, 3H), 4.22 (t, J=7.8 Hz, 2H), 3.07 (t, J=7.8 Hz, 2H), 2.30 (d, J=4.9 Hz, 3H).

### Step 2: 1-(5-bromo-7-fluoro-2,3-dihydro-1H-indol-1-yl)ethan-1-one

Bromine (0.391 ml, 7.652 mmol, 1.5 eq) was added dropwise to a cooled (0° C.) solution of 1-(7-fluoro-2,3-dihydro-1H-indol-1-yl)ethan-1-one (0.9141 g, 5.1012 mmol, 1.0 eq) in AcOH (34.01 ml, 0.15 M). Formation of precipitate was observed. Reaction mixture was stirred at rt for 5 h. UPLC analysis indicated unconsumed starting material and another portion of Bromine (0.07838 ml, 1.53 mmol, 0.3 eq) was added dropwise to a cooled (5° C.) mixture (total addition of Bromine—0.47 ml, 9.182 mmol, 1.8 eq). RM was stirred ON. Next, solid was filtered and washed with little amount of water, collected and dried under reduced pressure to give 1-(5-bromo-7-fluoro-2,3-dihydro-1H-indol-1-yl)ethan-1-one (1.08 g, 4.185 mmol, 82%) as a brown solid. LCMS: [C₁₀H₉BrFNO], desired mass=258.09, observed mass=260.1 [M+H]⁺. ¹H NMR (300 MHz, DMSO) δ 7.36 (d, J=10.2 Hz, 2H), 4.10 (t, J=8.0 Hz, 2H), 3.09 (t, J=7.9 Hz, 2H), 2.17 (d, J=1.9 Hz, 3H).

### Step 3: (1-acetyl-7-fluoro-2,3-dihydro-1H-indol-5-yl)boronic acid

Pressure vial was charged with 1-(5-bromo-7-fluoro-2,3-dihydro-1H-indol-1-yl)ethan-1-one (0.331 mmol, 1.0 eq), Tetrahydroxydiboron (0.045 g, 0.497 mmol, 1.5 eq) and Potassium acetate (0.098 g, 0.994 mmol, 3.0 eq), followed by addition of EtOH (3.31 ml, 0.1 M) and Ethylene glycol (0.056 ml, 0.994 mmol, 3.0 eq). Argon was bubbled through the mixture for 15 min, followed by addition of Dicyclohexyl[2-(2,4,6-triisopropylphenyl)phenyl]phosphane (0.016 g, 0.033 mmol, 0.1 eq) and (2-Dicyclohexylphosphino-2′,4′,6′-triisopropyl-1,1′-biphenyl)[2-(2′-amino-1,1′-biphenyl)]palladium(II) methanesulfonate (0.014 g, 0.017 mmol, 0.05 eq). Vial was tightly sealed and reaction mixture was stirred at 75° C. for 1 h. UPLC analysis indicated clean, full conversion to corresponding boronic acid derivative. The crude reaction mixture as a dark brown solution was taken to the next step without isolation. (1-acetyl-7-fluoro-2,3-dihydro-1H-indol-5-yl)boronic acid (0.074 g, 0.332 mmol, 100%). LCMS: [C₁₀H₁₁BFNO₃], desired mass=223.01, observed mass=224.20 [M+H]⁺.

### Step 4: 1-[5-(2-amino-5-methyl-1,3-thiazol-4-yl)-7-fluoro-2,3-dihydro-1H-indol-1-yl]ethan-1-one

To the (1-acetyl-7-fluoro-2,3-dihydro-1H-indol-5-yl)boronic acid (0.086 g, 0.387 mmol, 1.0 eq in 3.87 ml of EtOH) was added Potassium phosphate tribasic 1 M solution in water (0.247 g, 1.162 mmol, 3.0 eq; 1.17 ml, 0.33 M) and argon was bubbled through the mixture for 15 min, followed by addition of Dicyclohexyl[2-(2,4,6-triisopropylphenyl)phenyl]phosphane (0.0185 g, 0.039 mmol, 0.1 eq) and (2-Dicyclohexylphosphino-2′,4′,6′-triisopropyl-1,1′-biphenyl)[2-(2′-amino-1,1′-biphenyl)]palladium(II) methanesulfonate (0.0164 g, 0.019 mmol, 0.05 eq) and 4-Bromo-5-methylthiazol-2-amine (0.09 g, 0.465 mmol, 1.2 eq). Vial was tightly sealed and the mixture was stirred at 75° C. on. UPLC indicated the unconsumed starting material, therefore argon was bubbled through the mixture for 15 min and additional portions of Dicyclohexyl[2-(2,4,6-triisopropylphenyl)phenyl]phosphane (0.0092 g, 0.019 mmol, 0.05 eq) and Dicyclohexylphosphino-2′,4′,6′-triisopropyl-1,1′-biphenyl)[2-(2′-amino-1,1′-biphenyl)]palladium(II) methanesulfonate (0.0082, 0.0097 mmol, 0.025 eq) were added to the reaction mixture (Total addition of Dicyclohexyl[2-(2,4,6-triisopropylphenyl)phenyl]phosphane (0.0277 g, 0.058 mmol, 0.15 eq) and Dicyclohexylphosphino-2′,4′,6′-triisopropyl-1,1′-biphenyl)[2-(2′-amino-1,1′-biphenyl)]palladium(II) methanesulfonate (0.0246 g, 0.029 mmol, 0.075 eq)). Next, it was cooled down to rt and evaporated. Crude was purified by Flash Chromatography on silica gel (DCM/MeOH 100:0 to 70:30, dryload). NMR showed contamination with AcOH. DCM was added to DP and washed with sodium bicarbonate, solvent was evaporated and dried under vacuo to give 1-[5-(2-amino-5-methyl-1,3-thiazol-4-yl)-7-fluoro-2,3-dihydro-1H-indol-1-yl]ethan-1-one (0.066 g, 0.227 mmol, 59%). Brown solid. LCMS: [C₁₄H₁₄FN₃OS], desired mass=291.34, observed mass=292.3 [M+H]⁺, ¹H NMR (300 MHz, DMSO) δ 7.44-7.10 (m, 2H), 6.80 (s, 2H), 4.13 (t, J=7.8 Hz, 2H), 3.11 (t, J=7.9 Hz, 2H), 2.35 (s, 3H), 2.20 (d, J=2.3 Hz, 3H).

### Step 5: N-[4-(1-acetyl-7-fluoro-2,3-dihydro-1H-indol-5-yl)-5-methyl-1,3-thiazol-2-yl]-2-{3-[(5-{[2-(2,6-dioxopiperidin-3-yl)-1,3-dioxo-2,3-dihydro-1H-isoindol-4-yl]amino}pentyl)oxy]phenyl}acetamide

A mixture of 2-{3-[(5-{[2-(2,6-dioxopiperidin-3-yl)-1,3-dioxo-2,3-dihydro-1H-isoindol-4-yl]amino}pentyl)oxy]phenyl}acetic acid (0.1297 g, 0.2496 mmol, 1.1 eq), 1-[5-(2-amino-5-methyl-1,3-thiazol-4-yl)-7-fluoro-2,3-dihydro-1H-indol-1-yl]ethan-1-one (0.0661 g, 0.2269 mmol, 1.0 eq), DIPEA (0.1976 ml, 1.1344 mmol, 5.0 eq) and HATU (0.1035 g, 0.2723 mmol, 1.2 eq) in anhyd. DMF (2.27 ml, 0.1 M) and anhyd. ACN (2.27 ml, 0.1 M) (1:1) was stirred at rt for on. Due to incomplete consumption of starting material, an additional portion of HATU was added (0.1035 g, 0.2723 mmol, 1.2 eq) and reaction mixture was stirred ON. Total addition of HATU (0.2071 g, 0.5445 mmol, 2.4 eq). Then it was evaporated to dryness, dissolved in DCM and sat. Sodium bicarbonate was poured into the same flask. It was stirred for 15 minutes and then extracted with DCM, dried over anhyd. Sodium sulfate, filtered and evaporated to dryness. Then desired product was purified by prepHPLC to give 52.75 mg (0.069 mmol, 30% yield) of N-[4-(1-acetyl-7-fluoro-2,3-dihydro-1H-indol-5-yl)-5-methyl-1,3-thiazol-2-yl]-2-{3-[(5-{[2-(2,6-dioxopiperidin-3-yl)-1,3-dioxo-2,3-dihydro-1H-isoindol-4-yl]amino}pentyl)oxy]phenyl}acetamide as an yellow solid. LCMS: [C₄₀H₃₉FN₆O₇S], desired mass=766.85, observed mass=767.6 [M+H]⁺, 765.7 [M−H]⁻, ¹H NMR (300 MHz, DMSO) δ 12.30 (s, 1H), 11.09 (s, 1H), 7.57 (t, 1H), 7.40 (s, 1H), 7.30 (d, J=12.6 Hz, 1H), 7.23 (t, J=7.9 Hz, 1H), 7.11 (d, J=8.6 Hz, 1H), 7.02 (d, J=7.0 Hz, 1H), 6.93-6.80 (m, 2H), 6.57 (t, J=5.9 Hz, 1H), 5.05 (dd, J=12.7, 5.5 Hz, 1H), 4.14 (t, J=7.9 Hz, 2H), 3.97 (t, J=6.3 Hz, 2H), 3.71 (s, 2H), 3.13 (t, J=7.9 Hz, 2H), 2.93-2.85 (m, 1H), 2.73 (t, J=1.8 Hz, 1H), 2.66-2.58 (m, 2H), 2.47 (s, 3H), 2.32-2.25 (m, 1H), 2.20 (d, J=2.2 Hz, 3H), 2.06-1.96 (m, 1H), 1.86-1.70 (m, 2H), 1.70-1.58 (m, 2H), 1.57-1.43 (m, 2H).

### Example 64

### Synthesis of 2-{3-[2-(2-{[2-(2,6-dioxopiperidin-3-yl)-1,3-dioxo-2,3-dihydro-1H-isoindol-4-yl]amino}ethoxy)ethoxy]phenyl}-N-{5-methyl-4-[1-(2-methylpyridine-3-carbonyl)-2,3-dihydro-1H-indol-5-yl]-1,3-thiazol-2-yl}acetamide (Cpd. No. 76)

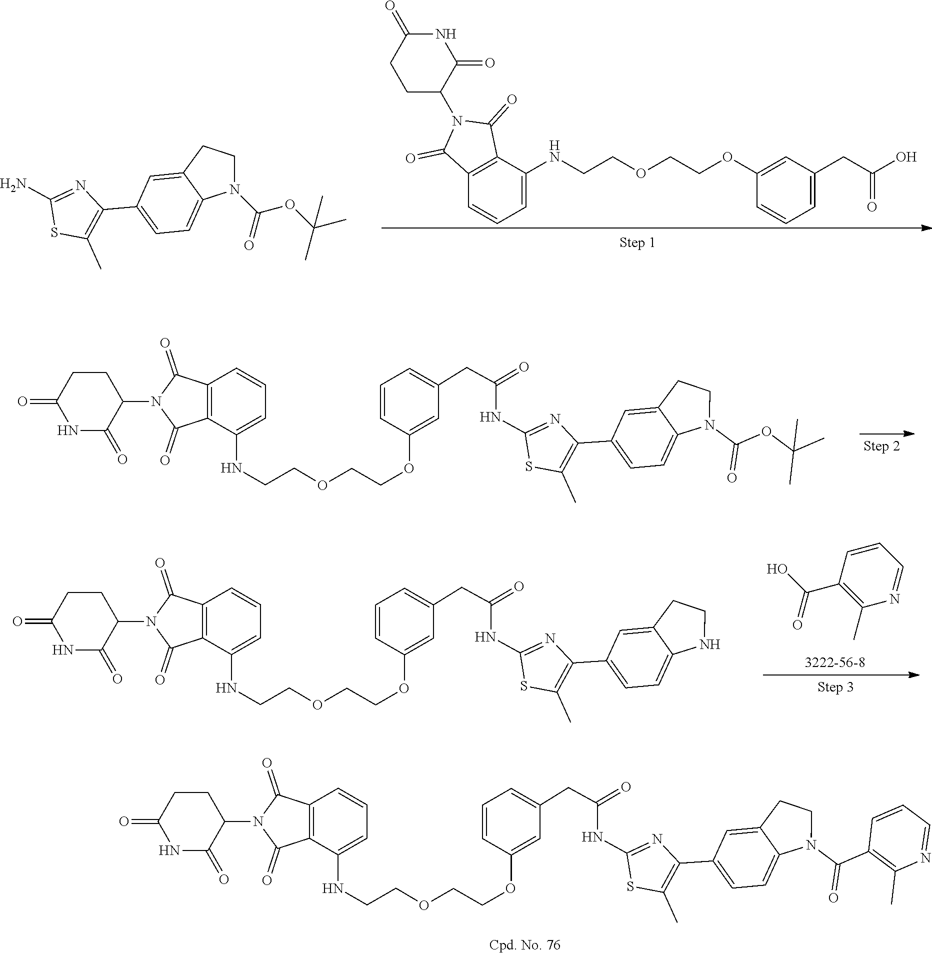

### Step 1: tert-butyl 5-[2-(2-{3-[2-(2-{[2-(2,6-dioxopiperidin-3-yl)-1,3-dioxo-2,3-dihydro-1H-isoindol-4-yl]amino}ethoxy)ethoxy]phenyl}acetamido)-5-methyl-1,3-thiazol-4-yl]-2,3-dihydro-1H-indole-1-carboxylate

To a solution of 2-{3-[2-(2-{[2-(2,6-dioxopiperidin-3-yl)-1,3-dioxo-2,3-dihydro-1H-isoindol-4-yl]amino}ethoxy)ethoxy]phenyl}acetic acid (1.011 g, 1.939 mmol, 1.1 eq) and tert-butyl 5-(2-amino-5-methyl-1,3-thiazol-4-yl)-2,3-dihydro-1H-indole-1-carboxylate (0.6 g, 1.763 mmol, 1.0 eq) in a 1:1 mixture of Dimethylformamide anhydrous (35 ml, 0.05 M):Acetonitrile anhydrous (35 ml, 0.05 M) was added 1-Methylimidazole (NMI) (0.724 g, 8.814 mmol, 5 eq). To this reaction mixture at 0° C. was added N,N,N′,N′-Tetramethylchloroformamidinium hexafluorophosphate (TCFH)(1.484 g, 5.288 mmol, 3.0 eq) and the reaction mixture was stirred at r.t. overnight. Solvents were evaporated, and crude was extracted using DCM and bicarbonate solution. After drying over Na₂SO₄, crude was purified by RP FC (ACN/H₂O+FA, loaded as a mixture of DMSO/ACN/H₂O) to give 1.62 g (quant. yield) of the title compound as a yellow solid. LCMS: [C₄₂H₄₄N₆O₉S], desired mass=808.91, observed mass=809.7 [M+H]⁺, ¹H NMR (300 MHz, DMSO-d6) δ 12.27 (s, 1H), 11.09 (s, 1H), 7.96 (s, 1H), 7.61-7.51 (m, 1H), 7.49-7.36 (m, 2H), 7.32-7.09 (m, 2H), 7.02 (dd, J=7.0, 3.0 Hz, 1H), 6.95-6.77 (m, 3H), 6.64 (t, J=6.0 Hz, 1H), 5.05 (dd, J=12.9, 5.3 Hz, 1H), 4.14-4.04 (m, 2H), 3.94 (t, J=8.7 Hz, 2H), 3.82-3.75 (m, 2H), 3.69 (s, 3H), 3.56-3.44 (m, 2H), 3.15-3.05 (m, 2H), 2.69-2.48 (m, 2.42 (s, 3H), 2.07-1.94 (m, 1H), 1.52 (s, 9H).

### Step 2: N-[4-(2,3-dihydro-1H-indol-5-yl)-5-methyl-1,3-thiazol-2-yl]-2-{3-[2-(2-{[2-(2,6-dioxopiperidin-3-yl)-1,3-dioxo-2,3-dihydro-1H-isoindol-4-yl]amino}ethoxy)ethoxy]phenyl}acetamide

To a solution of tert-butyl 5-[2-(2-{3-[2-(2-{[2-(2,6-dioxopiperidin-3-yl)-1,3-dioxo-2,3-dihydro-1H-isoindol-4-yl]amino}ethoxy)ethoxy]phenyl}acetamido)-5-methyl-1,3-thiazol-4-yl]-2,3-dihydro-1H-indole-1-carboxylate (1.62 g, 1.762 mmol, 1.0 eq) in dichloromethane (11.75 ml, 0.15 M) was added and stirred at room temperature for 1 h. The solvent was removed in vacuo to give 2.02 g (quant. yield) as a brown oil. Compound was dissolved in DCM and bicarbonate solution was added to it. The mixture was stirred for 30 minutes and the layers were separated. Organic layer was dried over Na₂SO₄, filtered and evaporated to give 1.15 g (87% yield) of the title compound as a free-base (yellow solid). LCMS: [C₃₇H₃₆N₆O₇S], desired mass=708.79, observed mass=709.6 [M+H]⁺, ¹H NMR (300 MHz, DMSO-d6) δ 12.29 (s, 1H), 11.09 (s, 1H), 7.67-7.45 (m, 3H), 7.34-7.10 (m, 3H), 7.03 (d, J=7.0 Hz, 1H), 6.94-6.76 (m, 3H), 6.64 (s, 1H), 5.05 (dd, J=12.8, 5.3 Hz, 1H), 4.13-4.04 (m, 3H), 3.83-3.63 (m, 7H), 3.58-3.40 (m, 2H), 3.24-3.12 (m, 2H), 2.64-2.53 (m, 2.44 (s, 3H), 2.06-1.90 (m, 1H).

### Step 3: 2-{3-[2-(2-{[2-(2,6-dioxopiperidin-3-yl)-1,3-dioxo-2,3-dihydro-1H-isoindol-4-yl]amino}ethoxy)ethoxy]phenyl}-N-{5-methyl-4-[1-(2-methylpyridine-3-carbonyl)-2,3-dihydro-1H-indol-5-yl]-1,3-thiazol-2-yl}acetamide

To a solution of N-[4-(2,3-dihydro-1H-indol-5-yl)-5-methyl-1,3-thiazol-2-yl]-2-{3-[2-(2-{[2-(2,6-dioxopiperidin-3-yl)-1,3-dioxo-2,3-dihydro-1H-isoindol-4-yl]amino}ethoxy)ethoxy]phenyl}acetamide (0.09 g, 0.121 mmol, 1.0 eq) and 2-Methylnicotinic acid (0.02 g, 0.145 mmol, 1.2 eq) in a 1:1 mixture of Dimethylformamide anhydrous (2.4 ml, 0.05 M):Acetonitrile anhydrous (2.4 ml, 0.05 M) was added 1-Methylimidazole (NMI) (0.05 g, 0.603 mmol, 5.0 eq). To this reaction mixture at 0° C. was added N,N,N′,N′-Tetramethylchloroformamidinium hexafluorophosphate (TCFH) (0.102 g, 0.362 mmol, 3.0 eq) and the reaction mixture was stirred at r.t. overnight. Solvents were evaporated, and crude was extracted using DCM and bicarbonate solution. After drying over Na₂SO₄, crude was submitted for prep. HPLC (FA) to give 0.056 g (54% yield) of the title compound as a yellow solid. LCMS: [C₄₄H₄₁N₇O₈S], desired mass=827.91, observed mass=828.8 [M+H]⁺, ¹H NMR (300 MHz, DMSO-d6) δ 12.32 (s, 1H), 11.08 (s, 1H), 8.56 (d, J=5.0 Hz, 1H), 8.24 (d, J=8.7 Hz, 1H), 7.85 (d, J=7.6 Hz, 1H), 7.61-7.52 (m, 3H), 7.38-7.31 (m, 1H), 7.22 (t, J=7.8 Hz, 1H), 7.15 (d, J=8.6 Hz, 1H), 7.02 (d, J=7.0 Hz, 1H), 6.95-6.80 (m, 3H), 6.64 (t, J=5.9 Hz, 1H), 5.05 (dd, J=12.9, 5.4 Hz, 1H), 4.15-4.05 (m, 2H), 3.84-3.74 (m, 4H), 3.74-3.65 (m, 3.50 (q, J=5.4 Hz, 2H), 3.42-3.33 (m, 1H), 3.15 (t, J=8.3 Hz, 2H), 2.95-2.81 (m, 2H), 2.62-2.52 (m, 2H), 2.46 (s, 3H), 2.41-2.33 (m, 1H), 2.06-1.94 (m, 1H).

### Example 65

### Synthesis of 2-{3-[2-(2-{[2-(2,6-dioxopiperidin-3-yl)-1,3-dioxo-2,3-dihydro-1H-isoindol-4-yl]amino}ethoxy)ethoxy]phenyl}-N-{5-methyl-4-[1-(3-methylpyridine-4-carbonyl)-2,3-dihydro-1H-indol-5-yl]-1,3-thiazol-2-yl}acetamide (Cpd. No. 77)

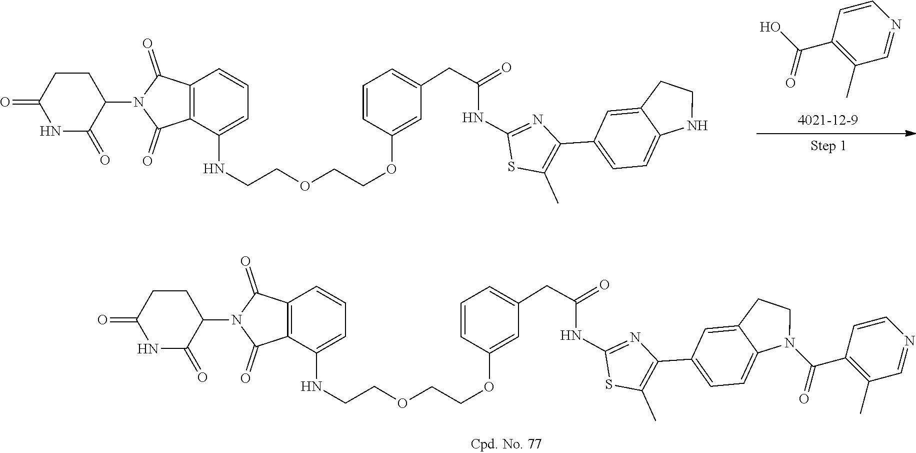

### Step 1: 2-{3-[2-(2-{[2-(2,6-dioxopiperidin-3-yl)-1,3-dioxo-2,3-dihydro-1H isoindol-4-yl]amino}ethoxy)ethoxy]phenyl}-N-{5-methyl-4-[1-(3-methylpyridine-4-carbonyl)-2,3-dihydro-1H-indol-5-yl]-1,3-thiazol-2-yl}acetamide

To a solution of N-[4-(2,3-dihydro-1H-indol-5-yl)-5-methyl-1,3-thiazol-2-yl]-2-{3-[2-(2-{[2-(2,6-dioxopiperidin-3-yl)-1,3-dioxo-2,3-dihydro-1H-isoindol-4-yl]amino}ethoxy)ethoxy]phenyl}acetamide (0.08 g, 0.107 mmol, 1.0 eq) and N,N,N′,N′-Tetramethylchloroformamidinium hexafluorophosphate (TCFH) (0.09 g, 0.322 mmol, 3.0 eq) in a 1:1 mixture of Dimethylformamide anhydrous (2.1 ml, 0.05 M):Acetonitrile anhydrous (2.1 ml, 0.05 M) were added 1-Methylimidazole (NMI) (0.044 g, 0.536 mmol, 5.0 eq) and 3-Methyl-4-pyridinecarboxylic acid (0.018 g, 0.129 mmol, 1.2 eq) and the reaction mixture was stirred at r.t. over the weekend. Solvents were evaporated, and crude was extracted using DCM and bicarbonate solution. After drying over Na₂SO₄, crude was submitted for prep. HPLC (FA) to give 0.017 g (19% yield) of the title compound as a yellow solid. LCMS: [C₄₄H₄₁N₇O₈S], desired mass=827.91, observed mass=828.7 [M+H]⁺, ¹H NMR (300 MHz, DMSO-d6) δ 12.34 (s, 1H), 11.10 (s, 1H), 8.60 (s, 1H), 8.54 (d, J=4.9 Hz, 1H), 8.23 (d, J=8.9 Hz, 1H), 7.61-7.52 (m, 3H), 7.44 (d, J=4.9 Hz, 1H), 7.23 (t, J=7.8 Hz, 1H), 7.15 (d, J=8.6 Hz, 1H), 7.03 (d, J=7.0 Hz, 1H), 6.95-6.81 (m, 3H), 6.65 (t, J=5.7 Hz, 1H), 5.06 (dd, J=12.9, 5.3 Hz, 1H), 4.13-4.04 (m, 2H), 3.86-3.76 (m, 3H), 3.74-3.64 (m, 3H), 3.54-3.45 (m, 2H), 3.29-3.23 (m, 2H), 3.16 (t, J=8.3 Hz, 2H), 2.95-2.80 (m, 1H), 2.63-2.53 (m, 2H), 2.46 (s, 3H), 2.32-2.24 (m, 3H), 2.05-1.96 (m, 1H).

### Example 66

### Synthesis of 2-{3-[2-(2-{[2-(2,6-dioxopiperidin-3-yl)-1,3-dioxo-2,3-dihydro-1H-isoindol-4-yl]amino}ethoxy)ethoxy]phenyl}-N-{5-methyl-4-[1-(4-methylpyridine-3-carbonyl)-2,3-dihydro-1H-indol-5-yl]-1,3-thiazol-2-yl}acetamide (Cpd. No. 78)

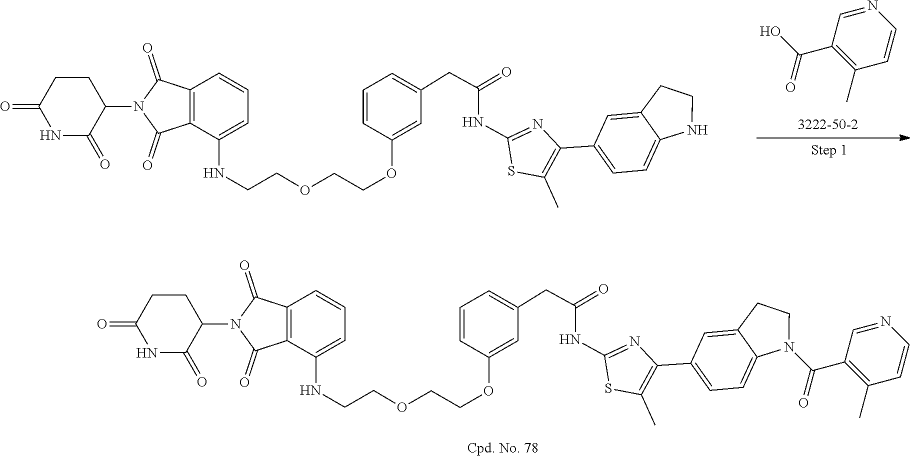

### Step 1: 2-{3-[2-(2-{[2-(2,6-dioxopiperidin-3-yl)-1,3-dioxo-2,3-dihydro-1H-isoindol-4-yl]amino}ethoxy)ethoxy]phenyl}-N-{5-methyl-4-[1-(4-methylpyridine-3-carbonyl)-2,3-dihydro-1H-indol-5-yl]-1,3-thiazol-2-yl}acetamide

To a solution of N-[4-(2,3-dihydro-1H-indol-5-yl)-5-methyl-1,3-thiazol-2-yl]-2-{3-[2-(2-{[2-(2,6-dioxopiperidin-3-yl)-1,3-dioxo-2,3-dihydro-1H-isoindol-4-yl]amino}ethoxy)ethoxy]phenyl}acetamide (0.08 g, 0.107 mmol, 1.0 eq) and N,N,N′,N′-Tetramethylchloroformamidinium hexafluorophosphate (TCFH) (0.09 g, 0.322 mmol, 3.0 eq) in a 1:1 mixture of Dimethylformamide anhydrous (2.1 ml, 0.05 M):Acetonitrile anhydrous (2.1 ml, 0.05 M) were added 1-Methylimidazole (NMI) (0.044 g, 0.536 mmol, 5.0 eq) and 4-methylpyridine-3-carboxylic acid (0.018 g, 0.129 mmol, 1.2 eq) and the reaction mixture was stirred at r.t. over the weekend. Solvents were evaporated, and crude was extracted using DCM and bicarbonate solution. After drying over Na₂SO₄, crude was submitted for prep. HPLC (FA) to give 0.016 g (18% yield) of the title compound as a yellow solid. LCMS: [C₄₄H₄₁N₇O₈S], desired mass=827.91, observed mass=828.7 [M+H]⁺, ¹H NMR (300 MHz, DMSO-d6) δ 12.34 (s, 1H), 11.10 (s, 1H), 8.60 (s, 1H), 8.54 (d, J=5.0 Hz, 1H), 8.25 (d, J=8.8 Hz, 1H), 7.61-7.51 (m, 3H), 7.41 (d, J=5.1 Hz, 1H), 7.23 (t, J=7.9 Hz, 1H), 7.16 (d, J=8.6 Hz, 1H), 7.03 (d, J=7.0 Hz, 1H), 6.97-6.81 (m, 3H), 6.66 (t, J=5.8 Hz, 1H), 5.06 (dd, J=12.8, 5.4 Hz, 1H), 4.15-4.06 (m, 2H), 3.88-3.76 (m, 3H), 3.75-3.65 (m, 3H), 3.55-3.45 (m, 2H), 3.30-3.24 (m, 2H), 3.16 (t, J=8.3 Hz, 2H), 2.96-2.82 (m, 1H), 2.64-2.55 (m, 2H), 2.47 (s, 3H), 2.34 (s, 3H), 2.06-1.94 (m, 1H).

### Example 67

### Synthesis of 2-{3-[2-(2-{[2-(2,6-dioxopiperidin-3-yl)-1,3-dioxo-2,3-dihydro-1H-isoindol-4-yl]amino}ethoxy)ethoxy]phenyl}-N-{5-methyl-4-[1-(3-methylpyridine-2-carbonyl)-2,3-dihydro-1H-indol-5-yl]-1,3-thiazol-2-yl}acetamide (Cpd. No. 79)

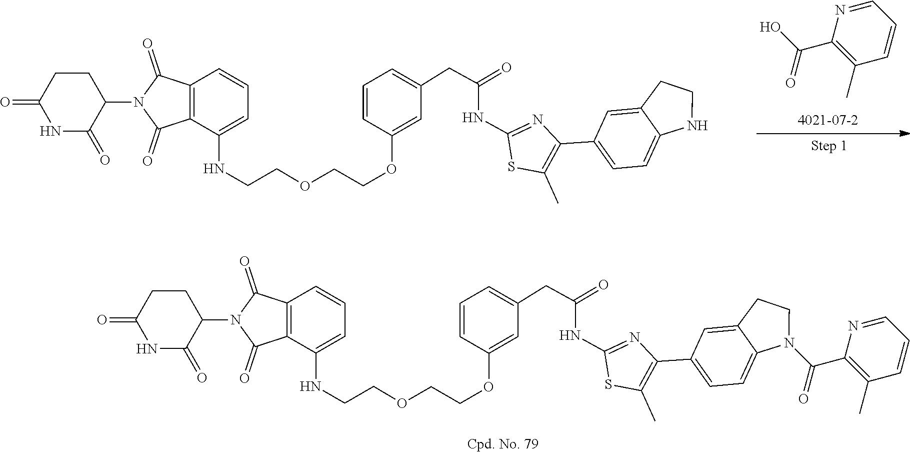

### Step 1: 2-{3-[2-(2-{[2-(2,6-dioxopiperidin-3-yl)-1,3-dioxo-2,3-dihydro-1H-isoindol-4-yl]amino}ethoxy)ethoxy]phenyl}-N-{5-methyl-4-[1-(3-methylpyridine-2-carbonyl)-2,3-dihydro-1H-indol-5-yl]-1,3-thiazol-2-yl}acetamide

To a solution of N-[4-(2,3-dihydro-1H-indol-5-yl)-5-methyl-1,3-thiazol-2-yl]-2-{3-[2-(2-{[2-(2,6-dioxopiperidin-3-yl)-1,3-dioxo-2,3-dihydro-1H-isoindol-4-yl]amino}ethoxy)ethoxy]phenyl}acetamide (0.08 g, 0.107 mmol, 1.0 eq) and N,N,N′,N′-Tetramethylchloroformamidinium hexafluorophosphate (TCFH) (0.09 g, 0.322 mmol, 3.0 eq) in a 1:1 mixture of Dimethylformamide anhydrous (2.1 ml, 0.05 M):Acetonitrile anhydrous (2.1 ml, 0.05 M) was added 1-Methylimidazole (NMI) (0.044 g, 0.536 mmol, 5.0 eq) and 3-Methylpicolinic acid (0.018 g, 0.129 mmol, 1.2 eq) and the reaction mixture was stirred at r.t. overnight. Solvents were evaporated, and crude was extracted using DCM and bicarbonate solution. After drying over Na₂SO₄, crude was submitted for prep. HPLC (FA) to give 0.018 g (19% yield) of the title compound as a yellow solid. LCMS: [C₄₄H₄₁N₇O₈S], desired mass=827.91, observed mass=828.7 [M+H]⁺, ¹H NMR (300 MHz, DMSO-d6) δ 12.33 (s, 1H), 11.10 (s, 1H), 8.48 (d, J=4.8 Hz, 1H), 8.25 (d, J=8.8 Hz, 1H), 7.83 (d, J=7.8 Hz, 1H), 7.57 (t, J=7.8 Hz, 3H), 7.45 (dd, J=7.9, 4.8 Hz, 1H), 7.27-7.13 (m, 2H), 7.03 (d, J=7.0 Hz, 1H), 6.96-6.82 (m, 3H), 6.70-6.60 (m, 1H), 5.06 (dd, J=12.9, 5.4 Hz, 1H), 4.14-4.07 (m, 2H), 3.88-3.65 (m, 7H), 3.56-3.47 (m, 2H), 3.16 (t, J=8.3 Hz, 2H), 2.96-2.81 (m, 1H), 2.64-2.56 (m, 2H), 2.47 (s, 3H), 2.46-2.39 (m, 1H), 2.34 (s, 3H), 2.07-1.94 (m, 1H).

### Example 68

### Synthesis of 2-{3-[2-(2-{[2-(2,6-dioxopiperidin-3-yl)-1,3-dioxo-2,3-dihydro-1H-isoindol-4-yl]amino}ethoxy)ethoxy]phenyl}-N-{4-[1-(4-methoxypyridine-3-carbonyl)-2,3-dihydro-1H-indol-5-yl]-5-methyl-1,3-thiazol-2-yl}acetamide (Cpd. No. 80)

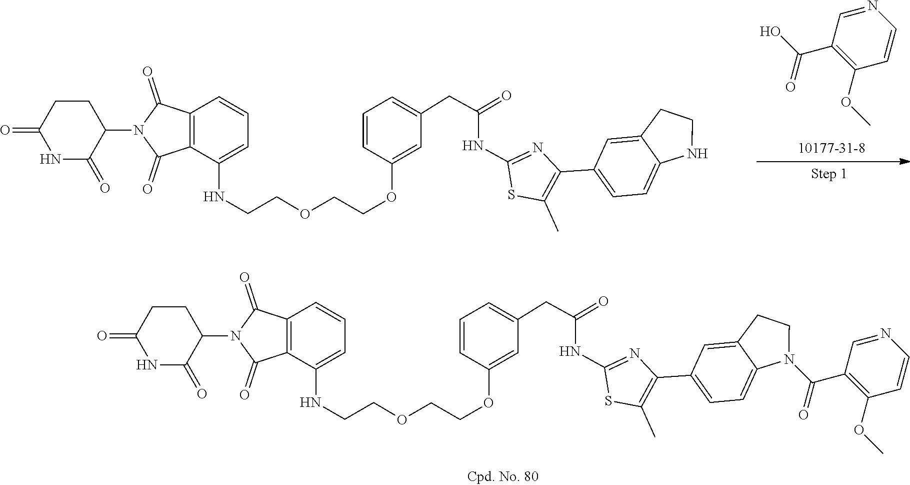 [SMILES: Cc1sc(NC(=O)Cc2cccc(OCCOCCNc3cccc4c3C(=O)N(C3CCC(=O)NC3=O)C4=O)c2)nc1-c1ccc2c(c1)CCN2 ; COc1ccncc1C(=O)O ; COc1ccncc1C(=O)N1CCc2cc(-c3nc(NC(=O)Cc4cccc(OCCOCCNc5cccc6c5C(=O)N(C5CCC(=O)NC5=O)C6=O)c4)sc3C)ccc21]

### Step 1: 2-{3-[2-(2-{[2-(2,6-dioxopiperidin-3-yl)-1,3-dioxo-2,3-dihydro-1H-isoindol-4-yl]amino}ethoxy)ethoxy]phenyl}-N-{4-[1-(4-methoxypyridine-3-carbonyl)-2,3-dihydro-1H-indol-5-yl]-5-methyl-1,3-thiazol-2-yl}acetamide

To a solution of N-[4-(2,3-dihydro-1H-indol-5-yl)-5-methyl-1,3-thiazol-2-yl]-2-{3-[2-(2-{[2-(2,6-dioxopiperidin-3-yl)-1,3-dioxo-2,3-dihydro-1H-isoindol-4-yl]amino}ethoxy)ethoxy]phenyl}acetamide (0.08 g, 0.107 mmol, 1.0 eq) and 4-Methoxynicotinic acid (0.02 g, 0.129 mmol, 1.2 eq) in a 1:1 mixture of Dimethylformamide anhydrous (2.1 ml, 0.05 M):Acetonitrile anhydrous (2.1 ml, 0.05 M) was added 1-Methylimidazole (NMI) (0.044 g, 0.536 mmol, 5.0 eq). To this reaction mixture at 0° C. was added N,N,N′,N′-Tetramethylchloroformamidinium hexafluorophosphate (TCFH) (0.09 g, 0.322 mmol, 3.0 eq) and the reaction mixture was stirred at r.t. overnight. Solvents were evaporated, and crude was extracted using DCM and bicarbonate solution. After drying over Na₂SO₄, crude was submitted for prep. HPLC (FA) to give 0.02 g (22% yield) of the title compound as a yellow solid. LCMS: [C₄₄H₄₁N₇O₉S], desired mass=843.91, observed mass=843.3 [M+H]⁺, ¹H NMR (300 MHz, DMSO-d6) δ 12.33 (s, 1H), 11.09 (s, 1H), 8.57 (d, J=5.8 Hz, 1H), 8.44 (s, 1H), 8.19 (d, J=8.9 Hz, 1H), 7.61-7.48 (m, 3H), 7.27-7.19 (m, 2H), 7.15 (d, J=8.6 Hz, 1H), 7.02 (d, J=7.0 Hz, 1H), 6.96-6.78 (m, 3H), 6.65 (t, J=5.8 Hz, 1H), 5.05 (dd, J=12.9, 5.3 Hz, 1H), 4.13-4.07 (m, 2H), 3.92 (s, 3H), 3.88-3.76 (m, 3.72-3.65 (m, 4H), 3.50 (q, J=5.6 Hz, 2H), 3.14 (t, J=8.3 Hz, 2H), 2.88 (ddd, J=17.6, 13.7, 5.3 Hz, 1H), 2.63-2.53 (m, 2H), 2.45 (s, 3H), 2.06-1.94 (m, 1H).

### Example 69

### Cancer Cell Viability

Representative Compounds of the Disclosure were tested in A549 (Vendor: ATCC; Cat #CCL-185; Description: lung, carcinoma) and/or LK-2 (Vendor: Riken; Cat #RCB1970; Description: lung, squamous cell carcinoma) cellular viability assays according to the following general protocol.
1. Observed the cells under microscope.
2. Aspirated the media and added 5 mL trypsin and incubate for 3 mins.
3. Added 10 mL fresh media and harvested all the cells in to 15 mL tubes.
4. Centrifuged the cells at 1000 rpm for 5 mins.
5. Re-suspended the cells with 11 mL media and took 1 mL to determine the cell number and viability.
6. Prepared cells suspension at 20 K/mL (A549) or 35 K/mL (LK-2)
7. Seeded the cells into 96-well plate and incubated for overnight.
8. Added compounds into each well with a HPD300 machine (nine three-fold dilutions typically starting at 30 M final concentration) and incubated for 3 days.
9. After 3 days, took out the plates from the incubator and observed the cells under microscope.
10. Passed a film at the bottom of the plates.
11. Equilibrated the plate and its contents at room temperature for approximately 30 minutes.
12. Thawed the CellTiter-Glo® Buffer and equilibrated to room temperature prior to use.
13. Equilibrated the lyophilized CellTiter-Glo® Substrate to room temperature prior to use.
14. Transferred 100 ml of CellTiter-Glo® Buffer into the amber bottle containing CellTiter-Glo® Substrate to reconstituted the lyophilized enzyme/substrate mixture.
15. Mixed by gently vortexing, swirling or inverting the contents to obtain a homogeneous solution.
16. Added 100 μL CellTiter-Glo® Reagent into each well
17. Mixed contents for 15 minutes on an orbital shaker to induce cell lysis.
18. Recorded luminescence with EnSpire machine.
19. Passed the raw data into the file and analyzed the data with XLfit software.

The results are provided in Table 4 (n.t.=not tested).

TABLE 4

Cellular IC₅₀ (μM)

| Cpd. No. | A549 | LK-2 |
|---|---|---|
| 1 | 0.1 | 0.04 |
| 2 | >30 | n.t. |
| 6 | >30 | >30 |
| 12 | ~30 | 0.3 |
| 18 | 0.1 | 0.04 |
| 19 | 11 | 2.5 |
| 20 | 0.3 | 0.06 |
| 21 | >30 | >30 |
| 22 | 0.3 | 0.06 |
| 23 | 0.09 | 0.02 |
| 24 | >30 | >30 |
| 25 | >30 | >30 |
| 26 | >30 | 0.8 |
| 27 | 0.08 | 0.03 |
| 28 | 0.03 | 0.01 |
| 29 | 0.05 | 0.01 |
| | 0.05 | 0.02 |
| 30 | 0.01 (n = 2) | 0.003 |
| 31 | >30 | >30 |
| 32 | >30 | >30 |
| 33 | >30 | >30 |
| 34 | 0.06 | 0.02 |
| 35 | 0.03 | 0.01 |
| 36 | 0.06 (n = 3) | 0.03 (n = 2) |
| 37 | 0.04 | 0.01 |
| 38 | 0.2 | 0.04 |
| 39 | >30 | >30 |
| 40 | 0.11 (n = 3) | 0.08 |
| 49 | 0.7 | 0.3 |
| 65 | 0.12 | n.t. |
| 66 | 0.16 | n.t. |
| 67 | 0.75 | n.t. |
| 68 | >30 | n.t. |
| 69 | 0.18 | n.t. |
| 71 | >30 | n.t. |
| 72 | 0.23 | n.t. |
| 73 | 0.15 | n.t. |
| 75 | 0.080 | n.t. |
| 76 | 0.011 | n.t. |
| 77 | 0.0077 | n.t. |
| 78 | 0.0059 | n.t. |
| 79 | 0.043 | n.t. |
| 80 | 0.075 | n.t. |
| 81 | >30 | n.t. |
| 83 | 0.045 | n.t. |
| 84 | 0.046 | n.t. |
| 85 | 0.047 | n.t. |
| 86 | 0.072 | n.t. |

### Example 70

### Protein Degradation Assay

Representative Compounds of the Disclosure were tested for protein degradation in huH1 cells (Vendor: JCRB: Cat #JCRB0199; Description: Human hepatoma) by Western blot analysis according to the following general protocol.

### Cell Treatment

Day 0: Plated the Huh1 cells into 10-cm dishes (1.5×10⁶/dish)

Day 1: Treated the cells with compounds at five concentrations, e.g., 0.01 μM, 0.04 μM, 0.11 μM, 0.33 μM and 1 μM; or 0.4 μM, 1.1 μM, 3.3 μM, 10 μM, and 30 μM.

Day 4: Harvested the cell pellets, spilt 1/3 of the pellet to extract RNA by Nucleospin RNA plus kit and measure Nrf2 and GPX2 by RT-PCR, the remaining 2/3 of the pellet to run Western blot analysis.

### Western Blot Assay Procedure

### Sample Preparation

a) Cell samples were lysed with lysis buffer (1×Cell lysis buffer added Protease inhibitor and phosphatase inhibitor) on ice for 5˜10 min and spun at 15,000 rpm in an Eppendorf microfuge for 15 min at 4° C.
b) The protein supernatant was collected in a new 1.5 ml tube and the protein concentrations were determined by the Pierce BCA kit (Thermo #23225).
c) Mixed protein sample with 5×SDS-PAGE loading buffer.
d) Boiled for 5 min.
e) Cooled at RT or on ice for 5 min.
f) Flash spun to bring down condensation prior to loading gel.

### Running the Gel

a) After flash spinning the samples, 20 μg protein were loaded into the wells.
b) Using SDS-PAGE running buffer.
c) Run with Constant Voltage (70 V) for 1.5-2 hr.
   Membrane transfer
a) Assembled “sandwich” for Invitrogen iBlot.
b) Transferred for 9 min using P0 program.
c) Immersed membrane in Odyssey blocking buffer or 5% BSA for at least 1 hr.

### Antibody Incubation and Gel Detection

a) Incubated with 1st Antibody 1:500 diluted in blocking buffer overnight at 4° C.
b) Washed 4×15 min with 0.05-0.1% Tween 20 in TBS.
c) Incubated with secondary antibody 1:10K diluted in blocking buffer for at least 1 hr at RT.
d) Washed 3×15 min with 0.05-0.1% Tween 20 in TBS.
e) Detected with ODYSSEY (Li—COR)
   The results are provided in Table 5.

TABLE 5

| | |
|---|---|
| Cpd. | DC₅₀ huHI1 cells |

| No. | <100 nM | 0.1-1 μM | 1-10 μM | >10 μM |
|---|---|---|---|---|
| 1 | | X | | |
| 2 | | | | X |
| 3 | | | | X |
| 4 | | | | X |
| 5 | | | | X |
| 7 | X | | | |
| 8 | | | | X |
| 12 | | X | | |
| 13 | | X | | |

### Example 71

### Measurement of Nrf2 Degradation in A549 HiBiT Nrf2 Cell Using Nano-Glo HiBiT Lytic Detection System

A549 HiBiT-Nrf2 cells (Promega, Cat. No. CS3023219) were seeded into 384-well plates at cell density of 5,000 cells/well in 40 μL of cell culture medium (Ham's F-12 Nutrient Mix supplemented with 10% FBS and 1% antibiotic-antimycotic).

The next day, cells were treated with representative Compounds of the Disclosure and incubated for 6 h and 20 h at 37° C., 5% CO₂. Staurosporine was used as a control.

At the end of incubation Nrf2 degradation (HiBiT readout) and cell viability (CellTiter-Glo readout) were determined. For HiBiT readout, Nano-Glo HiBiT Reagent mix (Promega, Cat. No. N3050) was added to wells, plates were incubated 2 min on an orbital shaker and incubated at room temperature in the dark for additional 30 minutes. At the end of incubation luminescence was measured using ultrasensitive luminescence mode (Envision, Perkin Elmer).

For CellTiter-Glo readout, CellTiter-Glo reagent (Promega, Cat. No. G7573) was added to wells of parallel plates. Plates were incubated on orbital shaker for 2 minutes and incubated at room temperature in the dark for additional 30 min. At the end of incubation luminescence was measured (EnVision, Perkin Elmer).

The HiBiT readout results are provided in Table 7.

TABLE 7

| Cpd. No. | DC₅₀ (nM) at 6 h | DC₅₀ (nM) at 20 h |
|---|---|---|
| 12 | + | + |
| 18 | ++ | + |
| 20 | ++ | ++ |
| 24 | + | + |
| 27 | +++ | +++ |
| 28 | +++ | +++ |
| 29 | +++ | +++ |
| 30 | +++ | + |
| 31 | + | + |
| 34 | +++ | +++ |
| 35 | +++ | + |
| 36 | +++ | +++ |
| 37 | +++ | +++ |
| 40 | +++ | +++ |
| 41 | +++ | +++ |
| 42 | + | n.d. |
| 44 | +++ | +++ |
| 46 | ++ | ++ |
| 47 | ++ | ++ |
| 48 | + | ++ |
| 49 | +++ | +++ |
| 50 | ++ | ++ |
| 51 | +++ | +++ |
| 65 | +++ | +++ |
| 66 | ++ | +++ |
| 67 | +++ | +++ |
| 68 | n.d. | +++ |
| 69 | +++ | +++ |
| 71 | + | ++ |
| 72 | n.d. | + |
| 73 | +++ | +++ |
| 76 | + | +++ |
| 77 | + | +++ |
| 78 | + | +++ |
| 79 | + | +++ |
| 80 | + | n.d. |

“+++” = <300 nM;
“++” = 300-3,000 nM;
“+” = >3,000 nM;
“n.d.” = not determined

### Example 72

### Cancer Cell Testing

The inhibitory activity of representative Compounds of the Disclosure was tested in various cancer cell lines according to the following protocol.

### Cell Seeding

Cells were harvested from flasks into cell culture media and cell numbers were counted. Cells were then diluted with culture medium to the desired density, and 40 μL of cell suspension were added into each well of a 384-well cell culture plate (Corning, #3764). Plates were covered with lids and placed at room temperature for 30 minutes without shaking, and then transferred into a 37° C. 5% CO₂ incubator.

### Compound Preparation and Treatment

Cpd. Nos. 30 and 34 were dissolved in DMSO to form a 10 mM stock solution. Paclitaxel as the control compound was dissolved in DMSO to form a 0.33 mM stock solution. 45 μL of stock solution was transferred to a 384 pp-plate and 3-fold, 10-point dilutions were performed by transferring 15 μL of compound into 30 μL of DMSO using a TECAN (EVO200) liquid handler. Plates were spun at room temperature at 1,000 RPM for 1 minute, and shaken at a plate shaker for 2 minutes. 120 mL of diluted compound were transferred from compound source plate into the cell plate. After compound treatment for 72 hours, CTG detection was performed for compound treatment plates as described in the “Detection” section.

### Detection

CellTiter Glo reagents were thawed and equilibrated to room temperature before the experiment. Plates were removed from incubators and equilibrated at room temperature for 15 minutes. 40 μL of CellTiter-Glo reagent was added to each well to be detected at 1:1 to culture medium. Then, plates were placed at room temperature for 30 min followed by reading on Ensight.

### Data Analysis

Inhibition activity was calculated following the formula below:

$$\% Inhibition = 100 \times ( {Readout}_{H C} - {Readout}_{S a m p l e} ) / ( {Readout}_{H C} - {Readout}_{L C} )$$

HC: 0.3% DMSO, LC: 1 μM Paclitaxel

IC_{50S} were calculated by fitting the Curve using Xlfit (v5.3.1.3) according to the following equation:

$$Y = Bottom + ( ( Top - Bottom ) / ( 1 + ( ( X / {IC}_{50} ) \hat{} HillSlope ) ) ) .$$

The results are provided in Table 8.

TABLE 8

| | | KEAP1 | NRF2 | Cpd. No. IC₅₀ (nM) | |
|---|---|---|---|---|---|
| Cell Line | Tumor Type | Mutation | Mutation | 30 | 34 |
| NCI-H920 | LUNG | p.G603V | | 191 | >30 μM |
| A549 | LUNG | p.G333C | | 4.3 | 20.6 |
| NCI-H2172 | LUNG | p.G430C | | 5.9 | 14.2 |
| NCI-H1944 | LUNG | p.R320L | | 25.2 | 106.9 |
| NCI-H2126 | LUNG | p.R320L | | 98.9 | >30 μM |
| NCI-H2030 | LUNG | p.V568F | | 169 | 224 |
| NCI-H1688 | LUNG | p.G511S | | <2 | 13 |
| NCI-H1792 | LUNG | p.G462W | | 6.5 | 41.7 |
| NCI-H1435 | LUNG | p.R413L | | 38 | 150 |
| HCC-15 | LUNG | p.G364C | | 4.1 | 30.1 |
| NCI-H2066 | LUNG | p.P361L | | 4.4 | 15.9 |
| SHP-77 | LUNG | p.D294Y | | 29.6 | 22.5 |
| NCI-H23 | LUNG | p.Q193H | | 126 | >30 μM |
| MORCPR | LUNG | p.K551SfsTer44 | | 3.4 | 33.2 |
| UMC11 | LUNG | p.G571A | | 5.8 | 12.2 |
| NCI-H322 | LUNG | p.R460S | | 3.9 | 23.9 |
| NCI-H2110 | LUNG | p.G429C | | 5.1 | 42.1 |
| LK-2 | LUNG | | p.E79K | <1.5 | 9.4 |
| NCI-H2228 | LUNG | | p.G31A | 12.6 | 57.4 |
| SNU2535 | LUNG | | p.D29H | 8.9 | >30 μM |
| EBC1 | LUNG | | p.D77V | 11.3 | 152.3 |
| CORL88 | LUNG | | p.Q26P | <1.5 | 4.40 |
| RERFLCSQ1 | LUNG | | p.D29H | 12.6 | 85.6 |
| NCI-H2122 | LUNG | p.A170_R204del | | 54.9 | >30 μM |
| NCI-H358 | LUNG | | | 11.6 | 44.9 |
| NCI-H460 | LUNG | p.D236H | | 15455 | >30 μM |
| HCC-95 | LUNG | | | 302 | >30 μM |
| HCC44 | UTERINE/ | p.F211C | | 397 | >30 μM |
| | ENDOMETRIAL | | | | |
| RL95-2 | UTERINE/ | p.R116W | | 2.8 | 20.9 |
| | ENDOMETRIAL | | | | |
| RMUG-S | UTERINE/ | p.G603V | | 21.5 | 18.1 |
| | ENDOMETRIAL | | | | |
| JHOC-5 | UTERINE/ | p.R483H | | 11.5 | 54.2 |
| | ENDOMETRIAL | | | | |
| HEC-265 | UTERINE/ | p.R483C | | 8.7 | 66.7 |
| | ENDOMETRIAL | | | | |
| HEC1A | UTERINE/ | p.Y33C | G31R | 2.9 | 20.3 |
| | ENDOMETRIAL | | | | |
| HEC251 | UTERINE/ | p.Q227R | p.K516T | <1.5 | 10.6 |
| | ENDOMETRIAL | | | | |
| HEC59 | UTERINE/ | | p.N329D | 2.8 | 20.4 |
| | ENDOMETRIAL | | | | |
| MESSA | UTERINE/ | | p.E79K | 4.7 | 35.1 |
| | ENDOMETRIAL | | | | |
| HEC1B | UTERINE/ | | p.G31R | 31.9 | >30 μM |
| | ENDOMETRIAL | | | | |
| HEC151 | UTERINE/ | p.A34S | p.L23GfsTer5 | 4.8 | 86.7 |
| | ENDOMETRIAL | | | | |
| KYSE520 | ESOPHAGEAL | | p.T80I | 1.7 | 13.2 |
| KYSE180 | ESOPHAGEAL | | p.P278Q | 4.0 | 45.8 |
| KYSE70 | ESOPHAGEAL | | p.W24C | 8.8 | 33.6 |
| OE21 | ESOPHAGEAL | | p.D318H | 156 | >30 μM |
| HO1U1 | HEAD & NECK | | p.E82D | 12.8 | 54.8 |
| SW579 | HEAD & NECK | | p.D77Y | 6.7 | 73.1 |
| MRC-5 | NORMAL LUNG | | | 12.5 | 35.4 |
| | FIBROBLAST | | | | |

It is to be understood that the foregoing embodiments and exemplifications are not intended to be limiting in any respect to the scope of the disclosure, and that the claims presented herein are intended to encompass all embodiments and exemplifications whether or not explicitly presented herein

All patents and publications cited herein are fully incorporated by reference in their entirety.

1. A compound of Formula A-I: 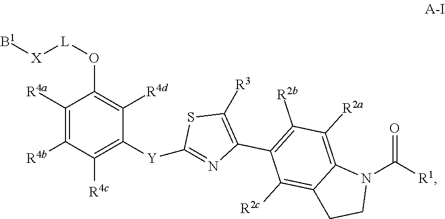
or a pharmaceutically acceptable salt or solvate thereof, wherein:
R¹ is selected from the group consisting of optionally substituted C₁-C₆ alkyl, optionally substituted C₃-C₈ cycloalkyl, optionally substituted phenyl, and optionally substituted 5- to 9-membered heteroaryl;
R^{2a}, R^{2b}, and R^{2c} are independently selected from the group consisting of hydrogen, C₁-C₄ alkyl, C₁-C₄ haloalkyl, C₁-C₄ alkoxy, C₁-C₄ haloalkoxy, cyano, hydroxy, —S(═O)₂CH₃, and halo;
R³ is selected from the group consisting of hydrogen, C₁-C₄ alkyl, C₁-C₄ haloalkyl, C₁-C₄ alkoxy, C₁-C₄ haloalkoxy, cyano, hydroxy, —S(═O)₂CH₃, and halo;
Y is selected from the group consisting of —\hat{ }N(H)C(═O)—, —\hat{ }N(H)C(═O)CH₂—, —\hat{ }C(═O)N(H)—and —\hat{ }C(═O)N(H)CH₂ —;
wherein the bond marked with a “\hat{ }” is attached to the thiazole;
R^{4a}, R^{4b}, R^{4c}, and R^{4d} are independently selected from the group consisting of hydrogen, C₁-C₄ alkyl, C₁-C₄ haloalkyl, C₁-C₄ alkoxy, C₁-C₄ haloalkoxy, cyano, hydroxy, —S(═O)₂CH₃, and halo;
L is selected from the group consisting of —(CH₂)ₘ—, —* (CH₂)ₙ(OCH₂CH₂)ₒ—, and —(CH₂)ₚ—Z—CH₂)_{q} —;
wherein the carbon marked with an “*” is attached to X;
m is 2, 3, 4, 5, 6, 7, 8, 9, 10, 11, or 12;
n is 2, 3, or 4;
o is 1, 2, 3, 4, 5, 6, 7, 8, 9, 10, 11, or 12;
p is 0, 1, 2, 3, 4, 5, 6, 7, 8, 9, 10, 11, or 12;
q is 1, 2, 3, 4, 5, 6, 7, 8, 9, 10, 11, or 12;
Z is selected from the group consisting of —(CR^{6a}R^{6b})— and —N(R⁷)—;
R^{6a} is selected from the group consisting of halo, hydroxyl, C₁-C₆ alkyl, C₁-C₄ haloalkyl, optionally substituted C₃-C₈ cycloalkyl, substituted optionally C₄-C₈ heterocyclo, optionally substituted phenyl, and optionally substituted 5- to 9-membered heteroaryl;
R^{6b} is selected from the group consisting of hydrogen and C₁-C₆ alkyl;
R⁷ is selected from the group consisting of hydrogen, C₁-C₆ alkyl, and C₁-C₄ haloalkyl;
X is selected from the group consisting of —O—, —NH—, 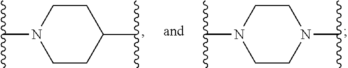 [SMILES: *C1CCN(*)CC1 ; *N1CCN(*)CC1]
B¹ is selected from the group consisting of: 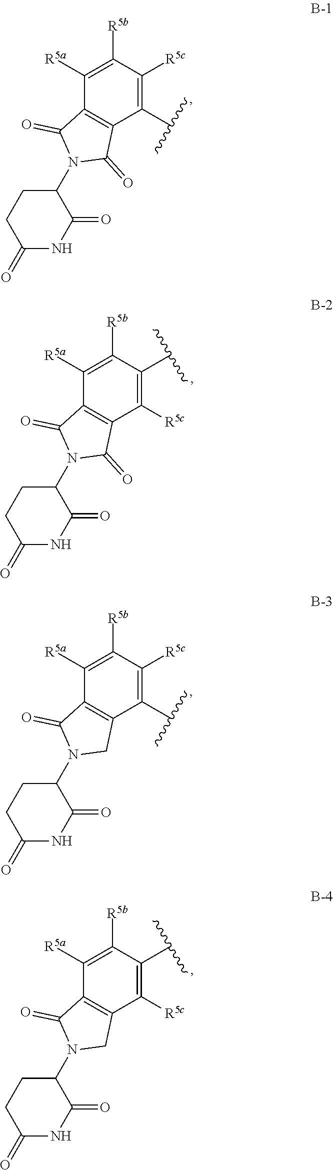 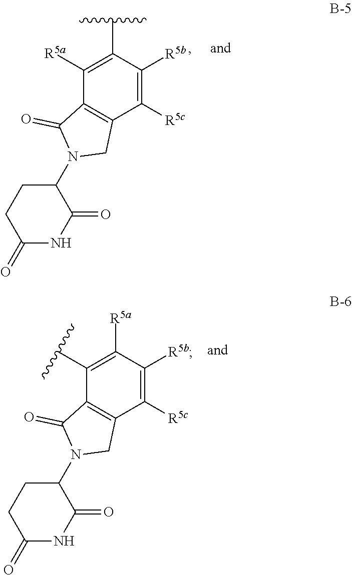
R^{5a}, R^{5b}, and R^{5c} are independently selected from the group consisting of hydrogen, C₁-C₄ alkyl, C₁-C₄ haloalkyl, C₁-C₄ alkoxy, C₁-C₄ haloalkoxy, cyano, hydroxy, —S(═O)₂CH₃, and halo.

2. The compound of claim 1 having Formula I: 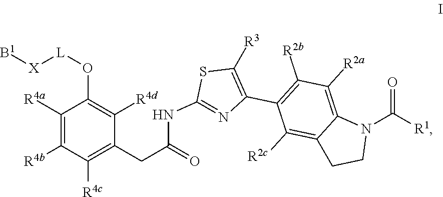
or a pharmaceutically acceptable salt or solvate thereof.

3. The compound of claim 1 having Formula II: 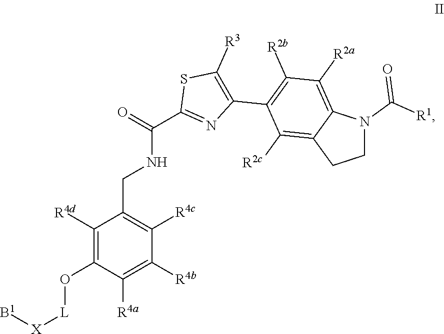
or a pharmaceutically acceptable salt or solvate thereof.

4-18. (canceled)

19. The compound of claim 1, or a pharmaceutically acceptable salt or solvate thereof, wherein L is selected from the group consisting of —(CH₂)ₘ— and —*(CH₂)ₙ(OCH₂CH₂)ₒ—.

20-24. (canceled)

25. The compound of claim 1, or a pharmaceutically acceptable salt or solvate thereof, wherein X is —O— or —NH—.

26-28. (canceled)

29. The compound of claim 1, or a pharmaceutically acceptable salt or solvate thereof, wherein B¹ is B-1, wherein R^{5a}, R^{5b}, and R^{5c} are hydrogen.

30. The compound of claim 1, or a pharmaceutically acceptable salt or solvate thereof, wherein B¹ is B-2, wherein R^{5a}, R^{5b}, and R^{5c} are hydrogen.

31-36. (canceled)

37. The compound of claim 1, or a pharmaceutically acceptable salt or solvate thereof, selected from the group consisting of:

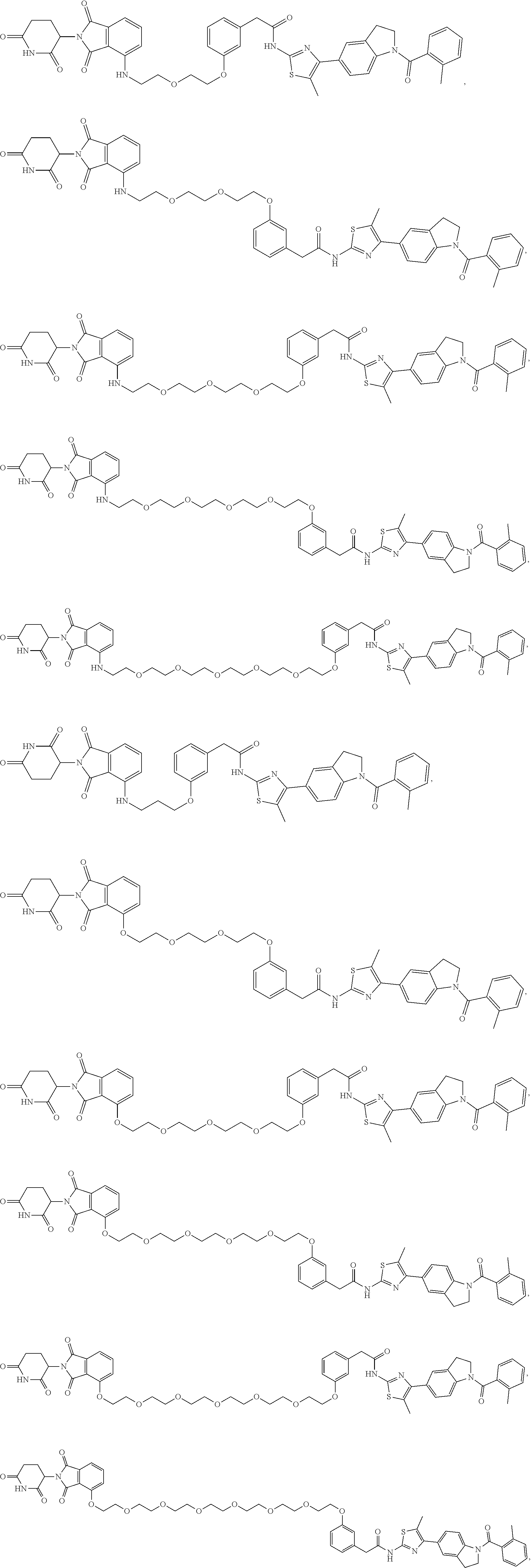

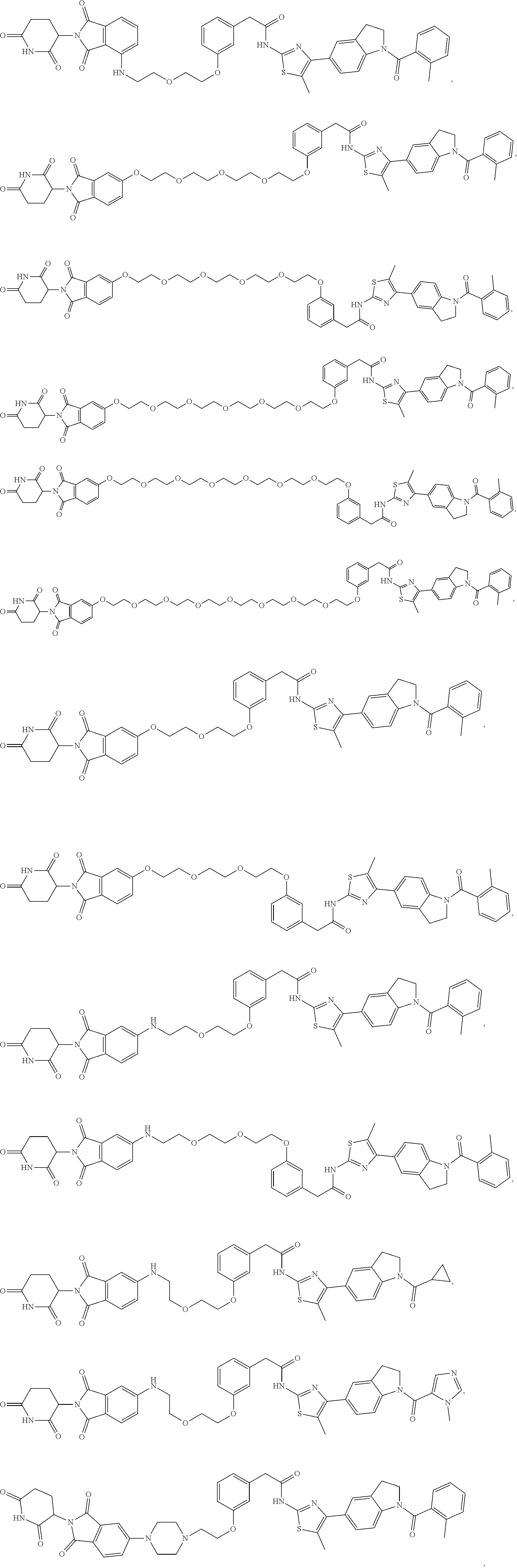

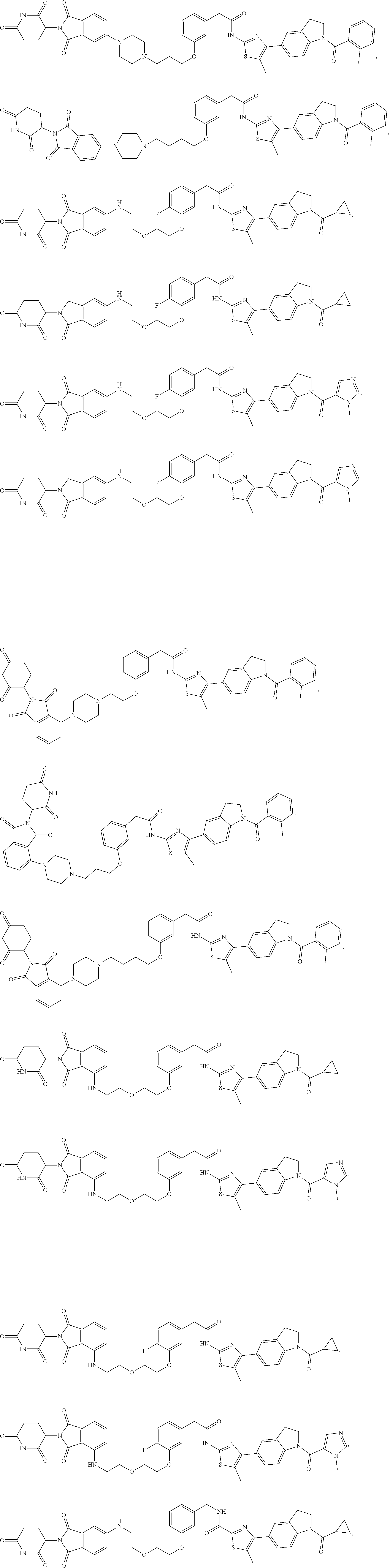

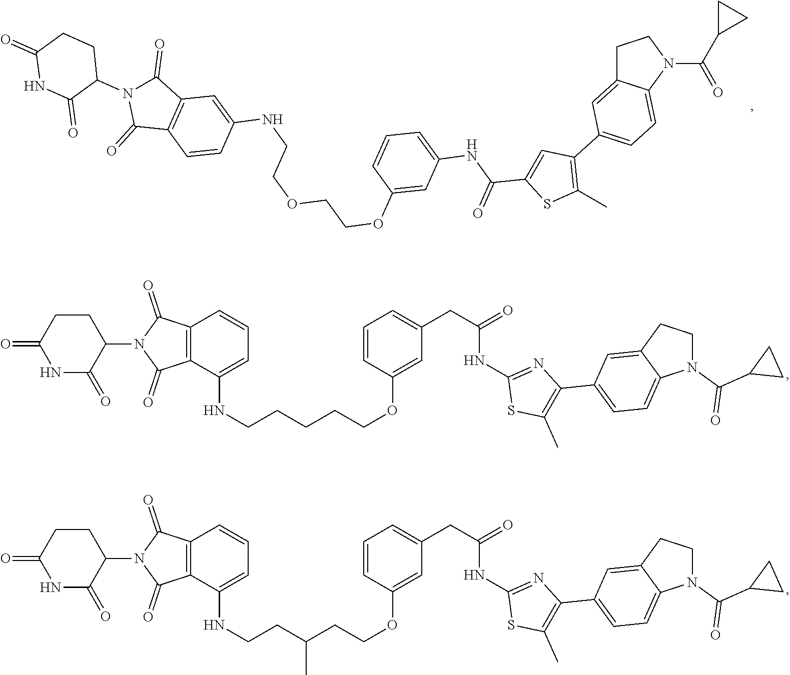 [SMILES: Cc1sc(C(=O)Nc2cccc(OCCOCCNc3ccc4c(c3)C(=O)N(C3CCC(=O)NC3=O)C4=O)c2)cc1-c1ccc2c(c1)CCN2C(=O)C1CC1 ; Cc1sc(NC(=O)Cc2cccc(OCCCCCNc3cccc4c3C(=O)N(C3CCC(=O)NC3=O)C4=O)c2)nc1-c1ccc2c(c1)CCN2C(=O)C1CC1 ; Cc1sc(NC(=O)Cc2cccc(OCCC(C)CCNc3cccc4c3C(=O)N(C3CCC(=O)NC3=O)C4=O)c2)nc1-c1ccc2c(c1)CCN2C(=O)C1CC1]

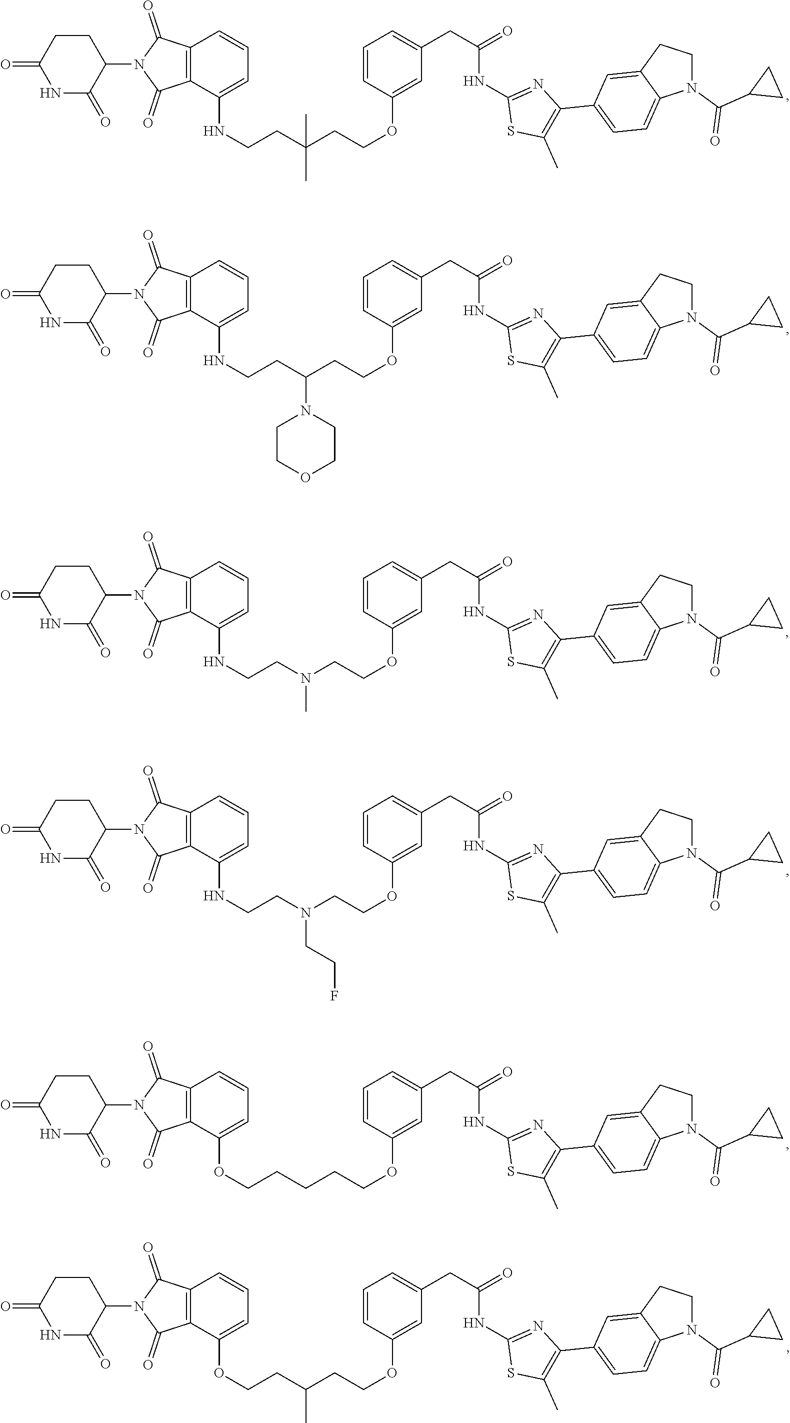

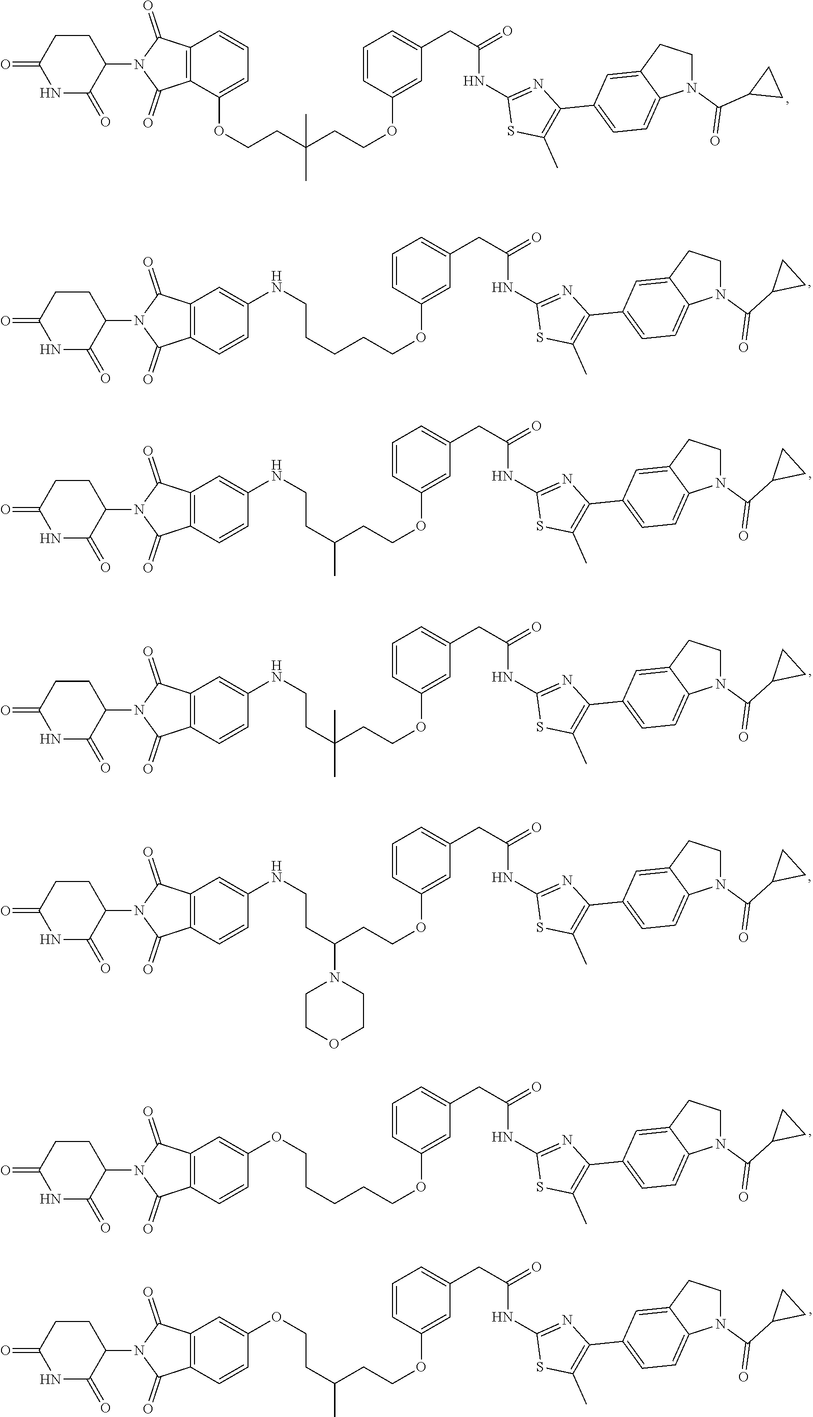

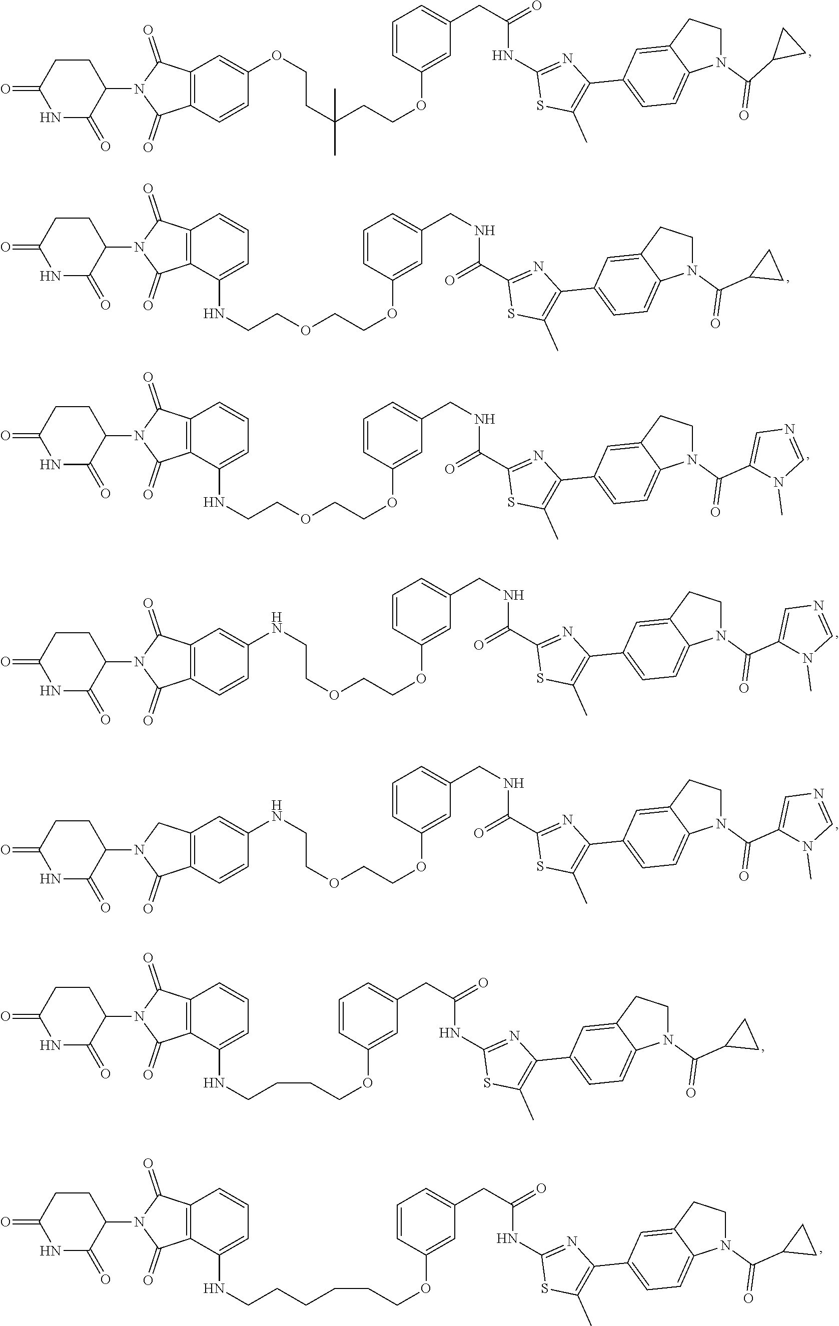

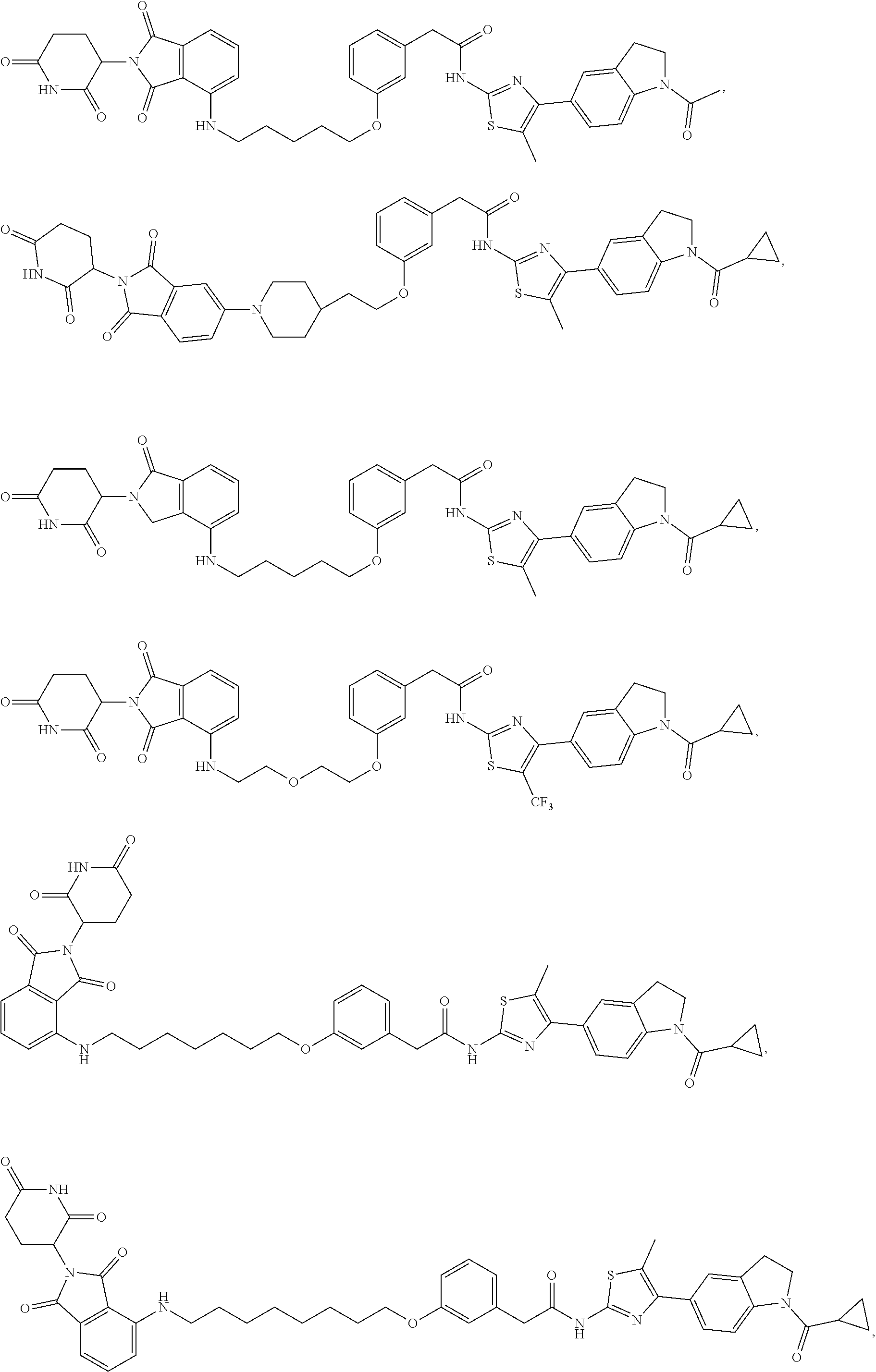

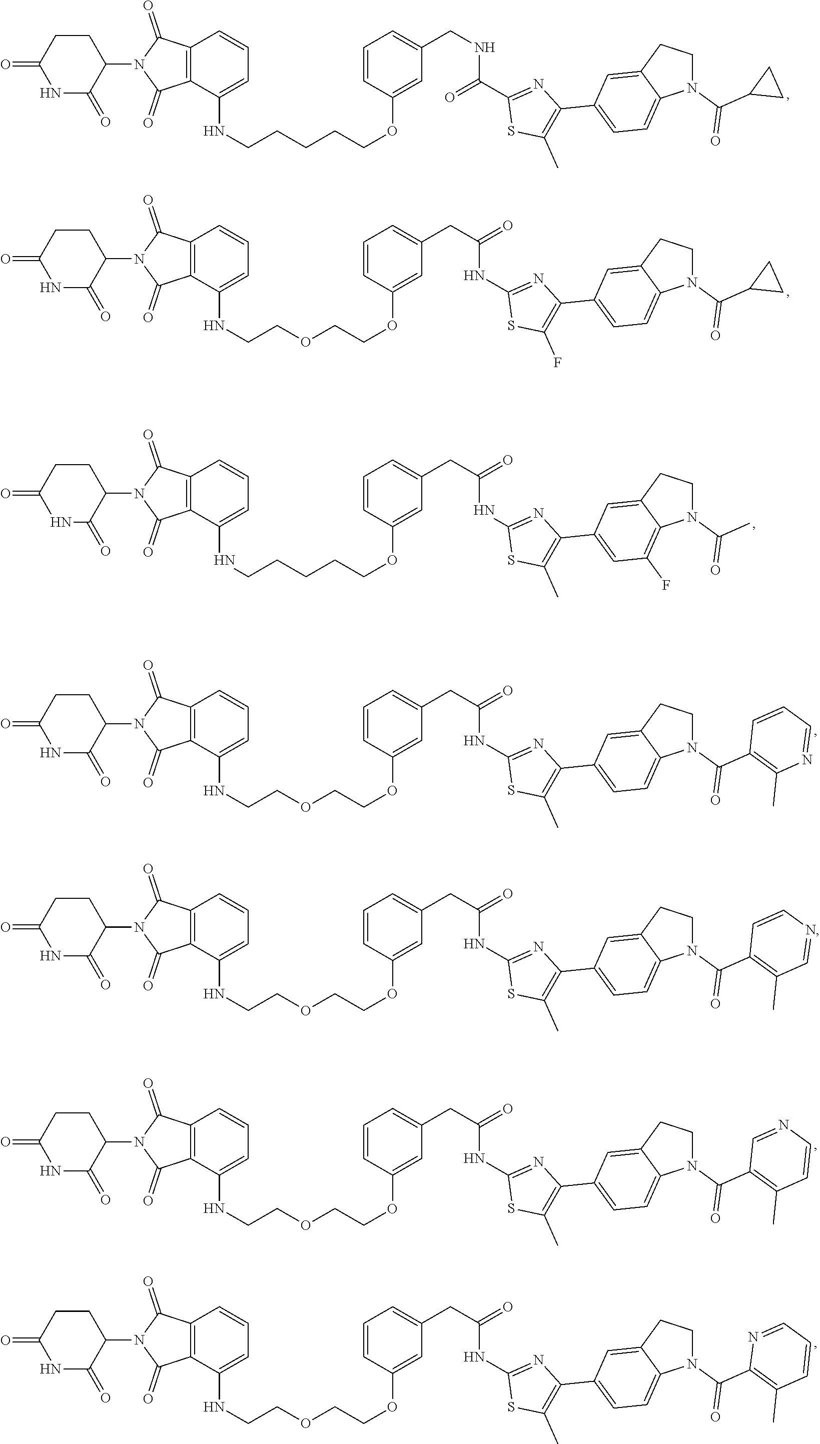

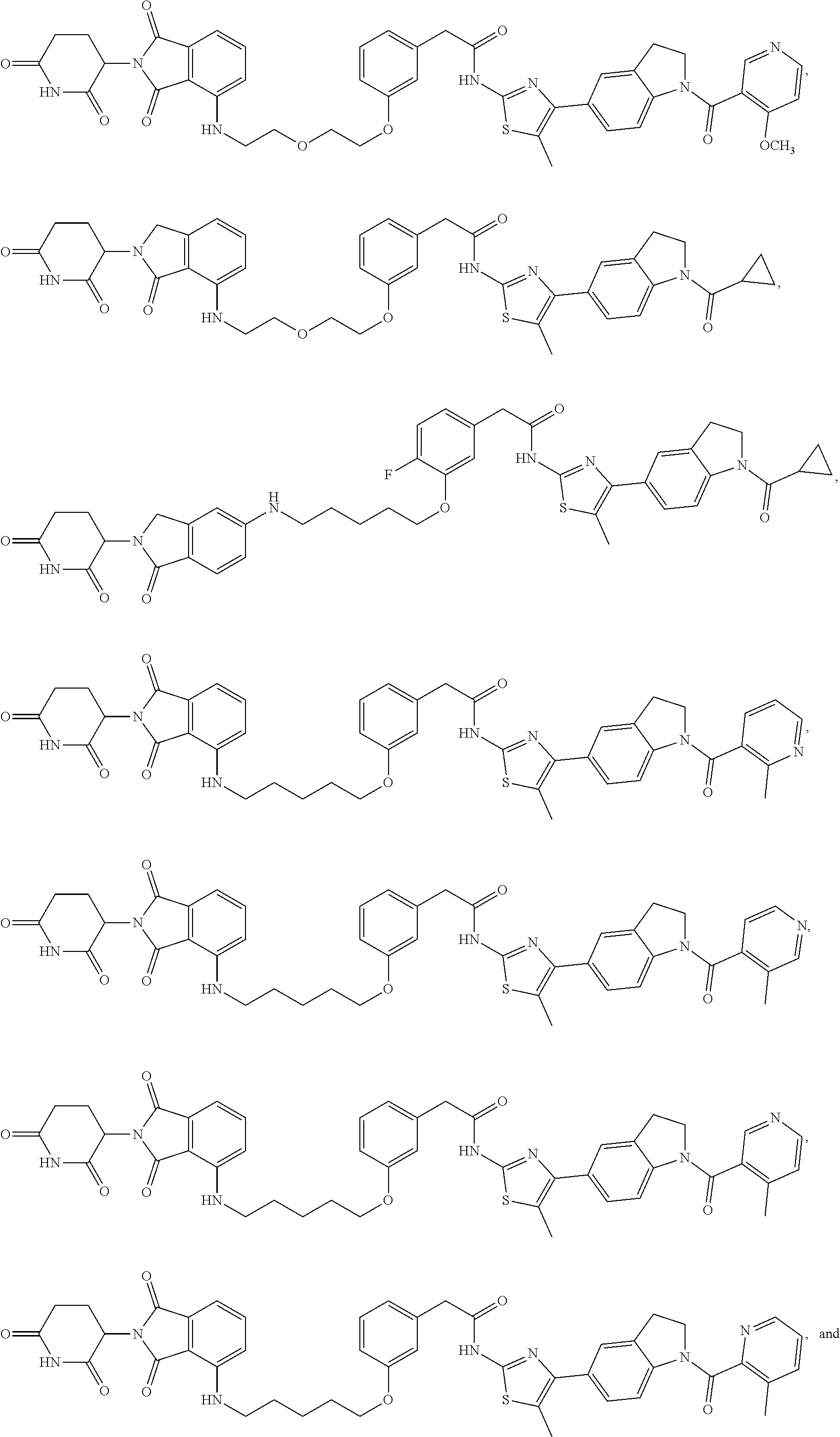

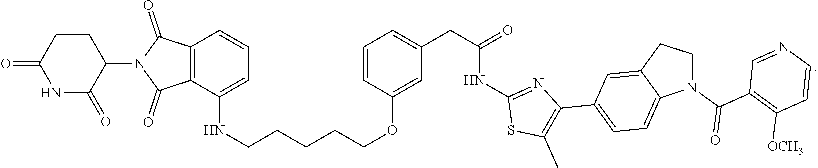 [SMILES: COc1ccncc1C(=O)N1CCc2cc(-c3nc(NC(=O)Cc4cccc(OCCCCCNc5cccc6c5C(=O)N(C5CCC(=O)NC5=O)C6=O)c4)sc3C)ccc21]

38. A pharmaceutical composition comprising the compound of claim 1, or a pharmaceutically acceptable salt thereof, and a pharmaceutically acceptable carrier.

39-47. (canceled)

48. A kit comprising the compound of claim 1, or a pharmaceutically acceptable salt thereof, and instructions for administering the compound, or a pharmaceutically acceptable salt thereof, to a subject having cancer.

49. A compound having Formula III: 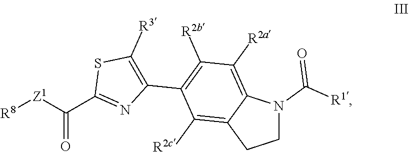
or a pharmaceutically acceptable salt thereof, wherein:
R^{1′} is selected from the group consisting of optionally substituted C₁-C₆ alkyl, optionally substituted C₃-C₈ cycloalkyl, optionally substituted phenyl, and optionally substituted 5- to 9-membered heteroaryl;
R^{2a′}, R^{2b′}, and R^{2c′} are independently selected from the group consisting of hydrogen, C₁-C₄ alkyl, C₁-C₄ haloalkyl, C₁-C₄ alkoxy, C₁-C₄ haloalkoxy, cyano, hydroxy, —S(═O)₂CH₃, and halo;
R^{3′} is selected from the group consisting of hydrogen, C₁-C₄ alkyl, C₁-C₄ haloalkyl, C₁-C₄ alkoxy, C₁-C₄ haloalkoxy, cyano, hydroxy, —S(═O)₂CH₃, and halo;
Z¹ is —O—; and
R⁸ is hydrogen, C₁-C₆ alkyl, or aralkyl; or
Z¹ is —N(H)—; and
R⁸ is: 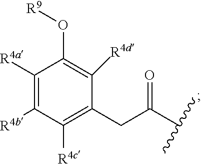
R^{4a′}, R^{4b′}, R^{4c′}, and R^{4d′} are independently selected from the group consisting of hydrogen, C₁-C₄ alkyl, C₁-C₄ haloalkyl, C₁-C₄ alkoxy, C₁-C₄ haloalkoxy, cyano, hydroxy, —S(═O)₂CH₃, and halo;
R⁹ is selected from the group consisting of hydrogen and -L¹-X¹;
X¹ is selected from the group consisting of —OR¹⁰ and —NR^{11a}R^{11b};
R¹⁰ is hydrogen;
R^{11a} is selected from the group consisting of hydrogen and —C(═O)OtBu;
R^{11b} is selected from the group consisting of hydrogen and C₁-C₄ alkyl;
L¹ is selected from the group consisting of —(CH₂)_{m′}—, —*(CH₂)ₙ(OCH₂CH₂)_{o′}—, and —(CH₂)_{p′}—Z²—(CH₂)_{q′}—;
wherein the carbon marked with an “*” is attached to X¹;
m′ is 2, 3, 4, 5, 6, 7, 8, 9, 10, 11, or 12;
n′ is 2, 3, or 4;
o′ is 1, 2, 3, 4, 5, 6, 7, 8, 9, 10, 11, or 12;
p′ is 0, 1, 2, 3, 4, 5, 6, 7, 8, 9, 10, 11, or 12;
q′ is 1, 2, 3, 4, 5, 6, 7, 8, 9, 10, 11, or 12;
Z² is selected from the group consisting of —(CR^{6a′}R^{6b′})— and —N(R^{7′})—;
R^{6a′} is selected from the group consisting of halo, hydroxyl, C₁-C₆ alkyl, C₁-C₄ haloalkyl, optionally substituted C₃-C₈ cycloalkyl, substituted optionally C₄-C₈ heterocyclo, optionally substituted phenyl, and optionally substituted 5- to 9-membered heteroaryl;
R^{6b′} is selected from the group consisting of hydrogen and C₁-C₆ alkyl; and
R⁷ is selected from the group consisting of hydrogen, C₁-C₆ alkyl, and C₁-C₄ haloalkyl.

50. A method of treating cancer in a subject comprising degrading nuclear factor erythroid 2-related factor 2 (Nrf2) in the subject in need thereof.

51. The method of claim 50, wherein degrading Nrf2 comprises administering a therapeutically effective amount of a Nrf2 degrader to the subject in need thereof.

52. The method of claim 51, wherein the Nrf2 degrader is a heterobifunctional small molecule comprising a ligand that binds to the Nrf2 protein and a ligand that binds to an E3 ligase.

53. The method of claim 52, wherein the Nrf2 degrader further comprises a linker that tethers the Nrf2 ligand and the E3 ligase ligand.

54. The method of claim 53, wherein the Nrf2 degrader is a compound of Formula A-I: 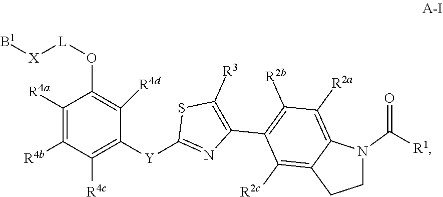
or a pharmaceutically acceptable salt or solvate thereof, wherein:
R¹ is selected from the group consisting of optionally substituted C₁-C₆ alkyl, optionally substituted C₃-C₅ cycloalkyl, optionally substituted phenyl, and optionally substituted 5- to 9-membered heteroaryl;
R^{2a}, R^{2b}, and R^{2c} are independently selected from the group consisting of hydrogen, C₁-C₄ alkyl, C₁-C₄ haloalkyl, C₁-C₄ alkoxy, C₁-C₄ haloalkoxy, cyano, hydroxy, —S(═O)₂CH₃, and halo;
R³ is selected from the group consisting of hydrogen, C₁-C₄ alkyl, C₁-C₄ haloalkyl, C₁-C₄ alkoxy, C₁-C₄ haloalkoxy, cyano, hydroxy, —S(═O)₂CH₃, and halo;
Y is selected from the group consisting of —\hat{ }N(H)C(═O)—, —\hat{ }N(H)C(═O)CH₂—, —\hat{ }C(═O)N(H)— and —\hat{ }C(═O)N(H)CH₂—;
wherein the bond marked with a “\hat{ }” is attached to the thiazole;
R^{4a}, R^{4b}, R^{4c}, and R^{4d} are independently selected from the group consisting of hydrogen, C₁-C₄ alkyl, C₁-C₄ haloalkyl, C₁-C₄ alkoxy, C₁-C₄ haloalkoxy, cyano, hydroxy, —S(═O)₂CH₃, and halo;
L is selected from the group consisting of —(CH₂)ₘ—, —*(CH₂)ₙ(OCH₂CH₂)ₒ—, and —(CH₂)ₚ—Z—CH₂)_{q}—;
wherein the carbon marked with an “*” is attached to X;
n is 2, 3, 4, 5, 6, 7, 8, 9, 10, 11, or 12;
n is 2, 3, or 4;
o is 1, 2, 3, 4, 5, 6, 7, 8, 9, 10, 11, or 12;
p is 0, 1, 2, 3, 4, 5, 6, 7, 8, 9, 10, 11, or 12;
q is 1, 2, 3, 4, 5, 6, 7, 8, 9, 10, 11, or 12;
Z is selected from the group consisting of —(CR^{6a}R^{6b})— and —N(R⁷)—;
R^{6a} is selected from the group consisting of halo, hydroxyl, C₁-C₆ alkyl, C₁-C₄ haloalkyl, optionally substituted C₃-C₈ cycloalkyl, substituted optionally C₄-C₈ heterocyclo, optionally substituted phenyl, and optionally substituted 5- to 9-membered heteroaryl;
R^{6b} is selected from the group consisting of hydrogen and C₁-C₆ alkyl;
R⁷ is selected from the group consisting of hydrogen, C₁-C₆ alkyl, and C₁-C₄ haloalkyl;
X is selected from the group consisting of —O—, —NH—, 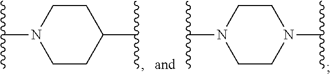 [SMILES: *C1CCN(*)CC1 ; *N1CCN(*)CC1 ; "and" ; "," ; ";"]
B¹ is selected from the group consisting of 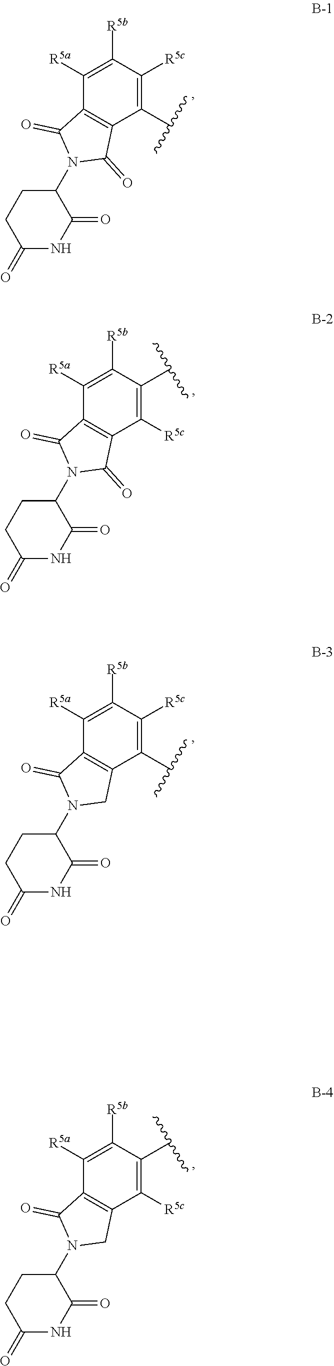 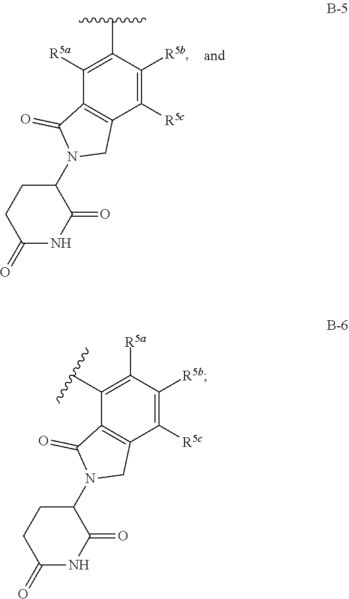
R^{5a}, R^{5b}, and R^{5c} are independently selected from the group consisting of hydrogen, C₁-C₄ alkyl, C₁-C₄ haloalkyl, C₁-C₄ alkoxy, C₁-C₄ haloalkoxy, cyano, hydroxy, —S(═O)₂CH₃, and halo.

55. A method of reducing Nrf2 protein levels within a cell of a subject, the method comprising administering a Nrf2 degrader to the subject.

56. The method of claim 55, wherein the Nrf2 degrader is a heterobifunctional small molecule comprising a ligand that binds to the Nrf2 protein and a ligand that binds to an E3 ligase.

57. The method of claim 56, wherein the Nrf2 degrader further comprises a linker that tethers the Nrf2 ligand and the E3 ligase ligand.

58. The method of claim 57, wherein the Nrf2 degrader is a compound of Formula A-I: 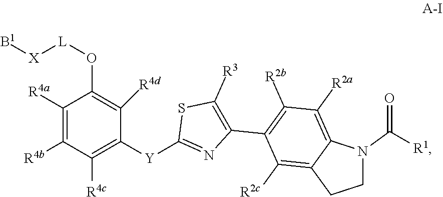
or a pharmaceutically acceptable salt or solvate thereof, wherein:
R¹ is selected from the group consisting of optionally substituted C₁-C₆ alkyl, optionally substituted C₃-C₅ cycloalkyl, optionally substituted phenyl, and optionally substituted 5- to 9-membered heteroaryl;
R^{2a}, R^{2b}, and R^{2c} are independently selected from the group consisting of hydrogen, C₁-C₄ alkyl, C₁-C₄ haloalkyl, C₁-C₄ alkoxy, C₁-C₄ haloalkoxy, cyano, hydroxy, —S(═O)₂CH₃, and halo;
R³ is selected from the group consisting of hydrogen, C₁-C₄ alkyl, C₁-C₄ haloalkyl, C₁-C₄ alkoxy, C₁-C₄ haloalkoxy, cyano, hydroxy, —S(═O)₂CH₃, and halo;
Y is selected from the group consisting of —\hat{ }N(H)C(═O)—, —\hat{ }N(H)C(═O)CH₂—, —\hat{ }C(═O)N(H)— and —\hat{ }C(═O)N(H)CH₂—;
wherein the bond marked with a “*” is attached to the thiazole;
R^{4a}, R^{4b}, R^{4c}, and R^{4d} are independently selected from the group consisting of hydrogen, C₁-C₄ alkyl, C₁-C₄ haloalkyl, C₁-C₄ alkoxy, C₁-C₄ haloalkoxy, cyano, hydroxy, —S(═O)₂CH₃, and halo;
L is selected from the group consisting of —(CH₂)ₘ—, —*(CH₂)ₙ(OCH₂CH₂)ₒ—, and —(CH₂)ₚ—Z—CH₂)_{q}—;
wherein the carbon marked with an “*” is attached to X;
m is 2, 3, 4, 5, 6, 7, 8, 9, 10, 11, or 12;
n is 2, 3, or 4;
o is 1, 2, 3, 4, 5, 6, 7, 8, 9, 10, 11, or 12;
p is 0, 1, 2, 3, 4, 5, 6, 7, 8, 9, 10, 11, or 12;
q is 1, 2, 3, 4, 5, 6, 7, 8, 9, 10, 11, or 12;
Z is selected from the group consisting of —(CR^{6a}R^{6b})— and —N(R⁷)—;
R^{6a} is selected from the group consisting of halo, hydroxyl, C₁-C₆ alkyl, C₁-C₄ haloalkyl, optionally substituted C₃-C₈ cycloalkyl, substituted optionally C₄-C₈ heterocyclo, optionally substituted phenyl, and optionally substituted 5- to 9-membered heteroaryl;
R^{6b} is selected from the group consisting of hydrogen and C₁-C₆ alkyl;
R⁷ is selected from the group consisting of hydrogen, C₁-C₆ alkyl, and C₁-C₄ haloalkyl;
X is selected from the group consisting of —O—, —NH—, 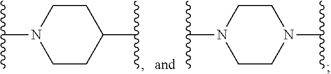 [SMILES: *C1CCN(*)CC1 ; *N1CCN(*)CC1 ; "and" ; "," ; ";"]
B¹ is selected from the group consisting of: 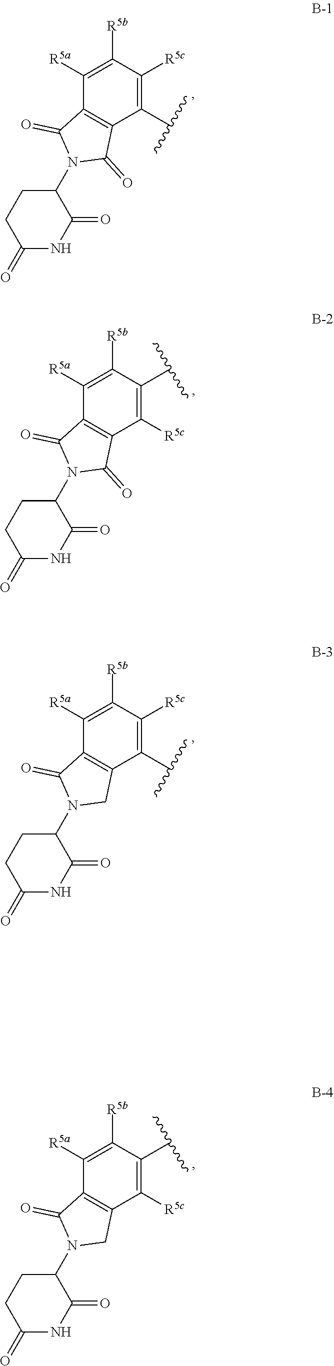 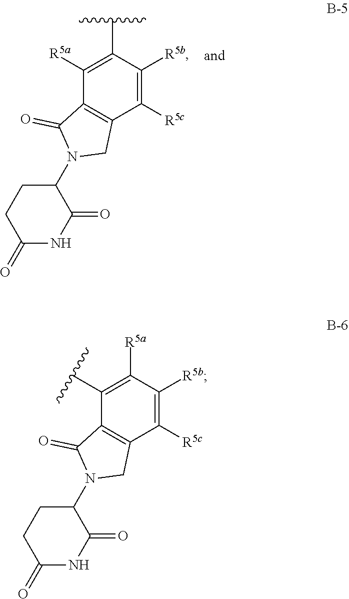
and
R^{5a}, R^{5b}, and R^{5c} are independently selected from the group consisting of hydrogen, C₁-C₄ alkyl, C₁-C₄ haloalkyl, C₁-C₄ alkoxy, C₁-C₄ haloalkoxy, cyano, hydroxy, —S(═O)₂CH₃, and halo
